(19) United States
(12) Patent Application Publication
(10) Pub. No.: US 2025/0147051 A1
(43) Pub. Date: May 8, 2025

(54) METHOD FOR DETECTING A TAU PROTEIN IN A SALIVA SAMPLE

(71) Applicant: GTINVENT LIMITED, Aberdeen Central Scotland (GB)

(72) Inventors: Richard LOFTHOUSE, Aberdeen (GB); Lewis Kirk PENNY, Aberdeen (GB); Mohammad ARASTOO, Aberdeen (GB); Soumya PALLIYIL SOMAN, Aberdeen (GB); Andrew Justin Radcliffe PORTER, Aberdeen (GB); Claude Michel WISCHIK, Aberdeen (GB)

(21) Appl. No.: 18/728,027

(22) PCT Filed: Jan. 12, 2023
(86) PCT No.: PCT/EP2023/050628

§ 371 Date: Jul. 10, 2024

(30) Foreign Application Priority Data
Jan. 12, 2022 (GB) 2200326.3

(51) Int. Cl. G01N 33/68; C07K 16/18; G01N 33/53

(52) U.S. Cl. CPC G01N 33/6896; C07K 16/18; G01N 33/53; G01N 2333/46

(57) ABSTRACT

The invention relates to an in vitro method for detecting a tau protein or fragment thereof in a saliva sample using a specific binding molecule, such as an antibody, directed to key epitopes of tau. The invention may find applications in diagnostics of tauopathies.

The invention relates to an in vitro method for detecting a tau protein or fragment thereof in a saliva sample using a specific binding molecule, such as an antibody, directed to key epitopes of tau. The invention may find applications in diagnostics of tauopathies.

Disorders related to tau are collectively referred to as neurodegenerative tauopathies. Alzheimer's disease (AD) is part of this group of neurodegenerative diseases. Conditions of dementia such as Alzheimer's disease (AD) are frequently characterised by a progressive accumulation of intracellular and/or extracellular deposits of proteinaceous structures such as β-amyloid plaques and neurofibrillary tangles (NFTs) composed of tau, in the brains of affected patients. The appearance of tau aggregation lesions largely correlates with pathological neurofibrillary degeneration and brain atrophy, as well as with cognitive impairment. In AD, tau protein self-assembles to form paired helical filaments (PHFs) and straight filaments that constitute the neurofibrillary tangles within neurons and dystrophic neurites in the brain. Protein misfolding to form amyloid fibrils is a hallmark of many different diseases collectively known as the amyloidoses, each of which is characterised by a specific precursor protein.

The long history of research into the causes of AD and other protein conformational disorders has not led to the hoped-for major advances in diagnostics or therapeutics. One reason for the limited progress is thought to be a lack of high-affinity specific binding molecules targeted to key epitopes of tau. This shortcoming was addressed as described in PCT application no. PCT/EP2021/069160 (incorporated herein by reference in its entirety), filed in the name of WisTa Laboratories Ltd., by the creation of the specific binding molecules disclosed therein. The disclosed specific binding molecules are derived from antibodies isolated from sheep immunised with full length tau protein and a truncated tau fragment from the core of the PHF. The use of sheep as a source of specific binding molecules is thought to have contributed to the high affinity of the specific binding molecules of the invention. PCT application no. PCT/EP2021/069160 describes the use of the specific binding molecules in a sample which may be a plasma sample, a whole blood sample, a brain lysate or a cerebrospinal fluid (CSF) sample.

H. Pekeles et al. Alzheimer's & Dementia: Diagnosis, Assessment & Disease Monitoring 11 (2019) 53-6054 describe development and validation of a salivary tau biomarker in Alzheimer's disease. The authors report a significant elevation of phosphorylated-tau to total-tau (p-tau/t-tau) ratio for the S396 phosphorylation site. However, the authors acknowledge a large variation in the AD salivary tau levels limits the utility of this test as a clinical biomarker.

The present inventors have developed assays for detecting a tau protein or fragment thereof in a saliva sample comprising contacting the sample with a first specific binding molecule wherein the first specific binding molecule binds to the predominant fragment isolated from proteolytically stable core of the paired helical filament (PHF) (residues 297 to 391 of SEQ ID NO: 1). The assays may use one or more specific binding molecule described in PCT application no. PCT/EP2021/069160, which may provide advantages including a high affinity leading to increased sensitivity. The present inventors have surprisingly identified that the use of a first specific binding molecule that binds to residues 297 to 391 of SEQ ID NO: 1 enables robust detection of salivary tau by a second specific binding molecule, but that the reverse use (a first specific binding molecule binding outside residues 297 to 391 of SEQ ID NO: 1 and a second specific binding molecule binding within residues 297 to 391 of SEQ ID NO: 1) can fail to detect salivary tau, even when a high affinity specific binding molecule that binds to residues 297 to 391 of SEQ ID NO: 1 is used.

### SUMMARY OF THE INVENTION

According to a first aspect, the invention provides an in vitro method for detecting a tau protein or fragment thereof in a saliva sample comprising contacting the sample with a first specific binding molecule wherein the first specific binding molecule binds to an epitope within residues 297 to 391 of SEQ ID NO: 1.

According to a second aspect, the invention provides a device for use in a method according to the first aspect.

According to a third aspect, the invention provides a kit comprising a specific binding molecule suitable for use in a method according to the first aspect and reagents for detecting a tau protein or fragment thereof in a saliva sample wherein the first specific binding molecule binds to an epitope within residues 297 to 391 of SEQ ID NO: 1.

Reference is made to a number of Figures as follows:

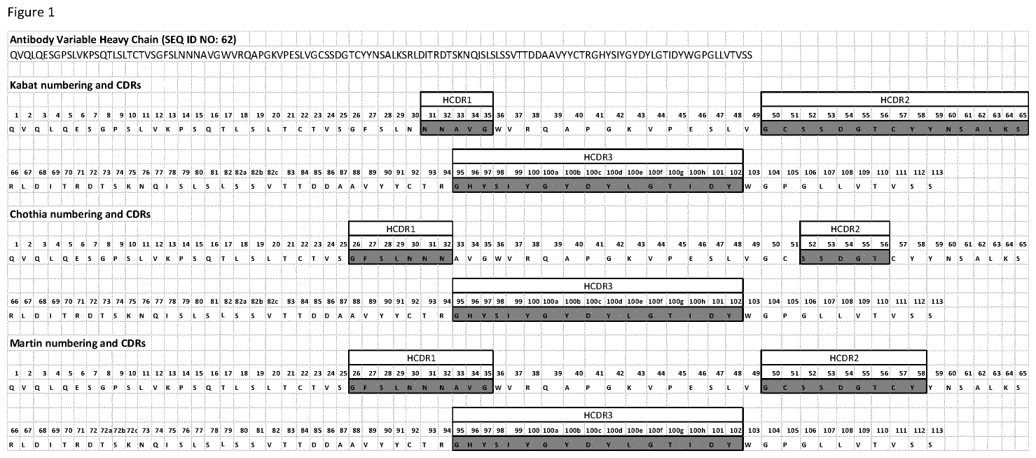

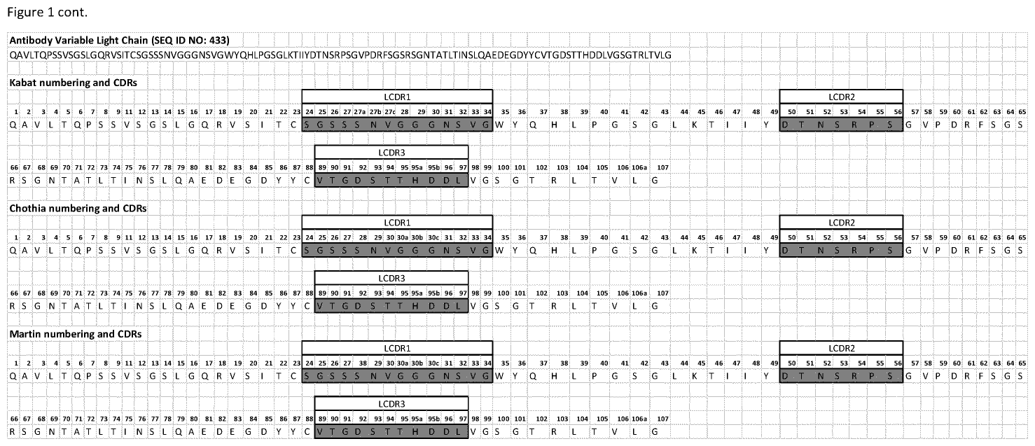

FIG. 1. Alternative CDR definitions for S1D12 according to Kabat, Chothia and Martin.

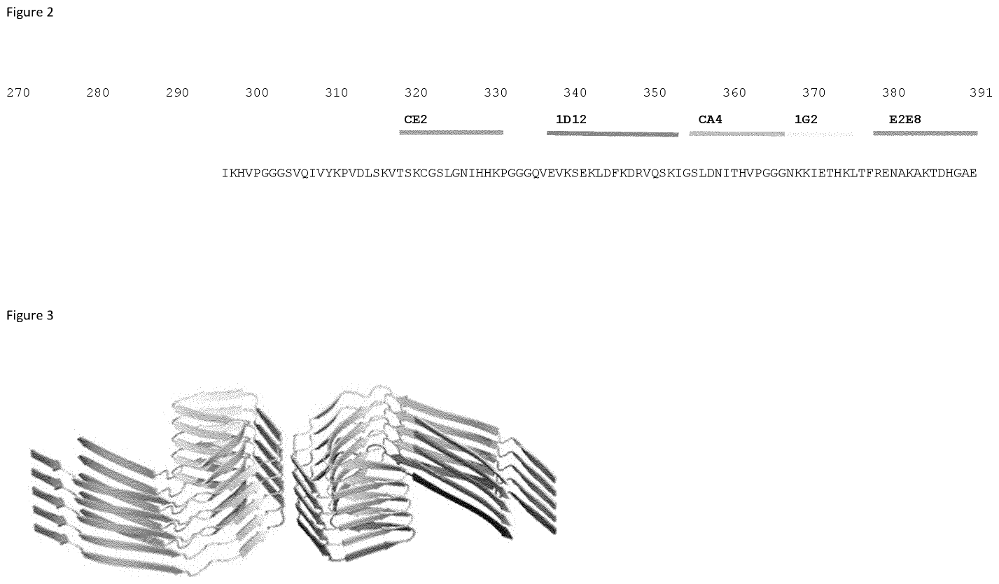

FIG. 2. The sequence of the predominant fragment isolated from the proteolytically stable core of the paired helical filament (PHF; Wischik et al., 1988). This fragment (referred to ‘dGAE’) comprises residues 296-391 of full-length tau and encompasses the fragment identified by cryo-electron-microscopy (residues 308-378) as constituting the PHF core (Fitzpatrick et al., 2017) and shown in FIG. 3. The locations of the epitopes of the selected antibodies/scAbs are also shown

FIG. 3. The PHF core shown in the context of a PHF.

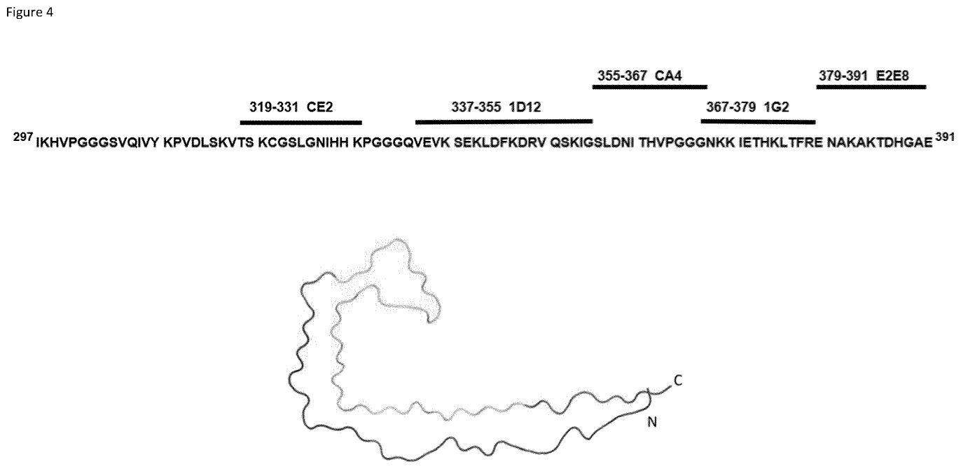

FIG. 4. The same core sequence and locations of corresponding epitopes in relation to the fundamental C-shaped subunit structure of the core. The 1D12 epitope forms the critical fold or “hairpin” of the C-shaped subunit.

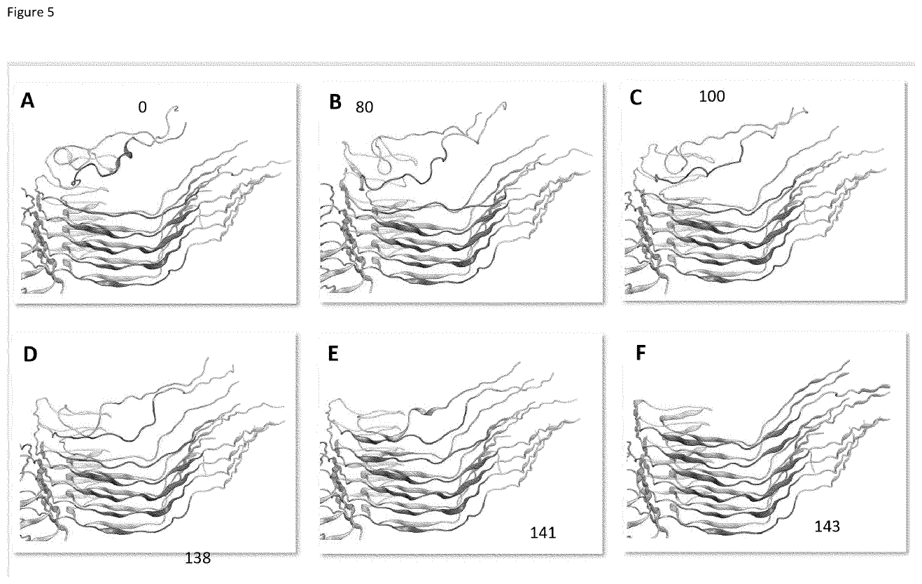

FIG. 5. Molecular modelling showing how a new dGAE unit progressively unfolds and becomes aligned with the structure of the existing oligomer.

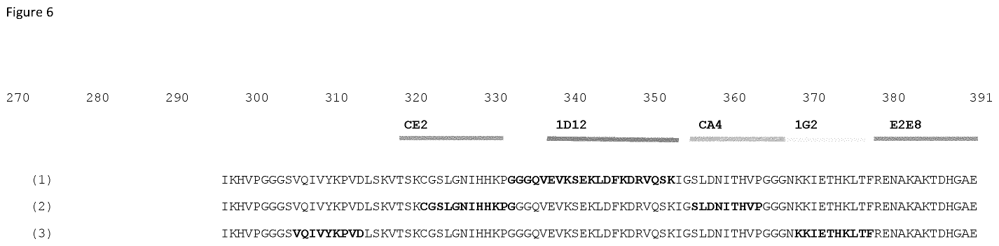

FIG. 6. The attachment sequence shown in terms of 3 stages corresponding to progressive binding of key segments of dGAE and their epitopes into the oligomer. As can be seen, the hinge region recognised by 1D12 is the primary site of attachment, followed by progressive symmetrical binding of the other domains.

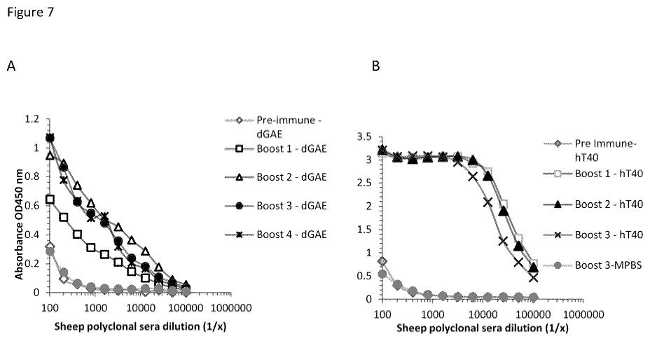

FIG. 7. (A) dGAE antigen specific immune response of sheep polyclonal sera after various rounds of immunisation. (B) hT40 antigen specific immune response of sheep polyclonal sera after various rounds of immunisation. MPBS coated wells included as negative control.

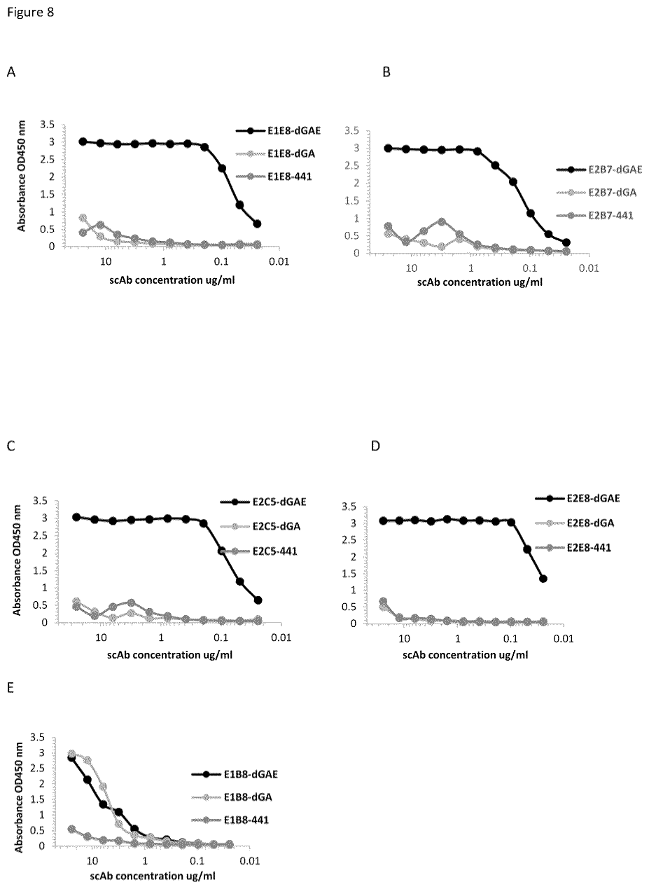

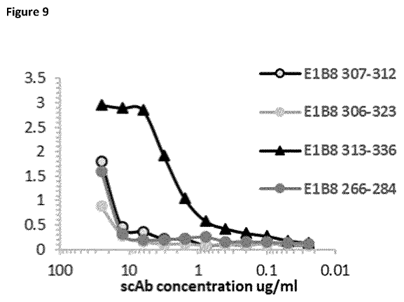

FIG. 8. ELISA based characterisation of the cross-reactivity of ‘E’ group scAbs using hT40, dGA and dGAE antigens (A) E1E8 scAb, (B) E2B7 scAb, (C) E2C5 scAb, (D) E2E8 scAb, (E) E1B8 scAb. All these scAbs except E1B8 showing specific dGAE binding and therefore requires C terminally accessible ‘391E’ epitope for immunoreactivity. E1B8 cross-reacts with dGA and a detailed mapping of its binding region is shown in FIG. 9

FIG. 9. Detailed mapping of E1B8 scAb which shows specific binding to the tau peptide representing amino acids from 313-336 on hT40 protein.

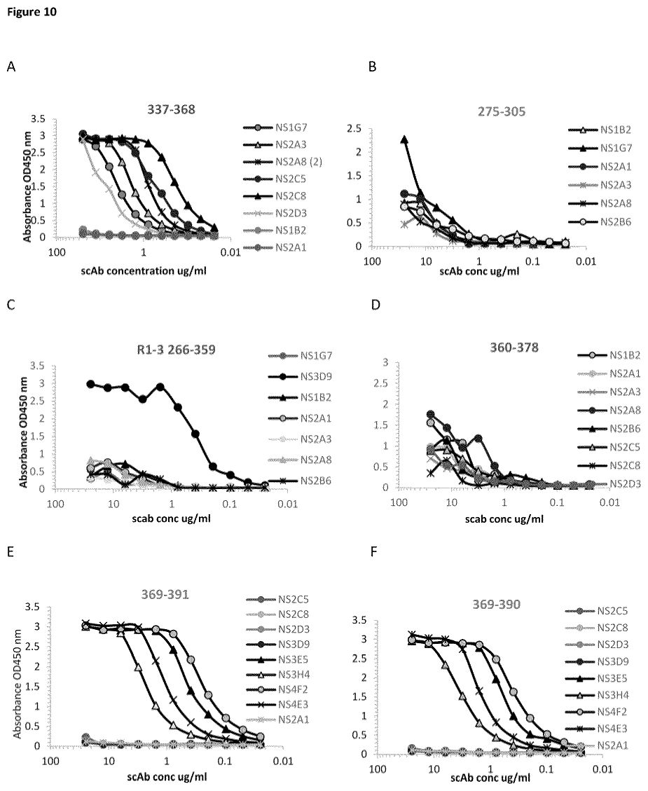

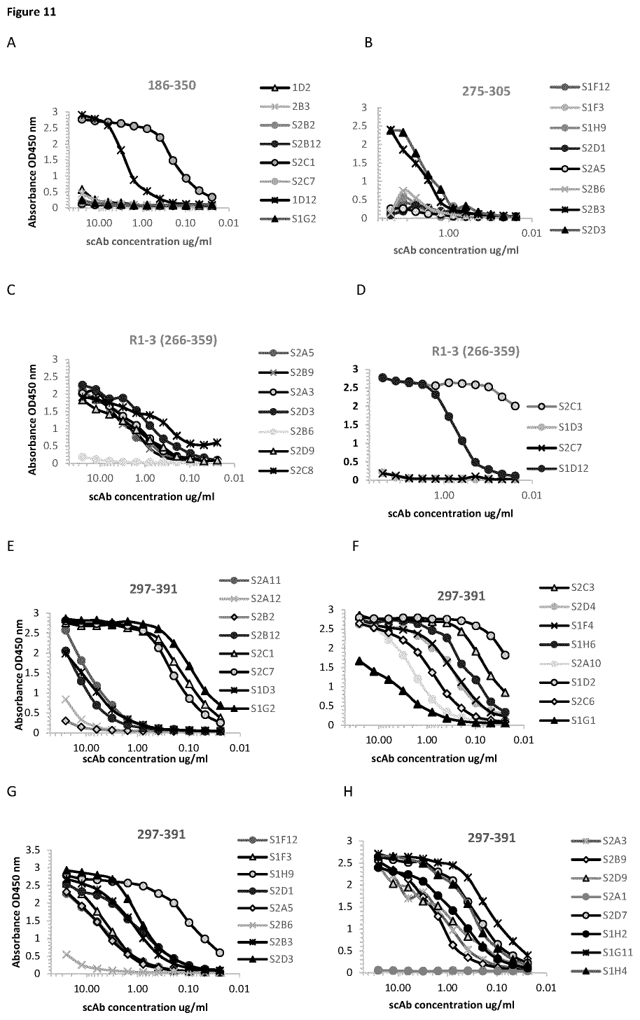

FIG. 10. ELISA based characterisation of the cross-reactivity of ‘NS’ group scAbs using various short tau fragments with numbers corresponding to hT40 amino acid residues. (A) 337-368, (B) 275-305 (C) 266-359 (R1-3) (D) 360-378 (E) 369-391, (F) 369-390. A summary of specific NS scAb binding to these shorter antigens are shown in Table 16

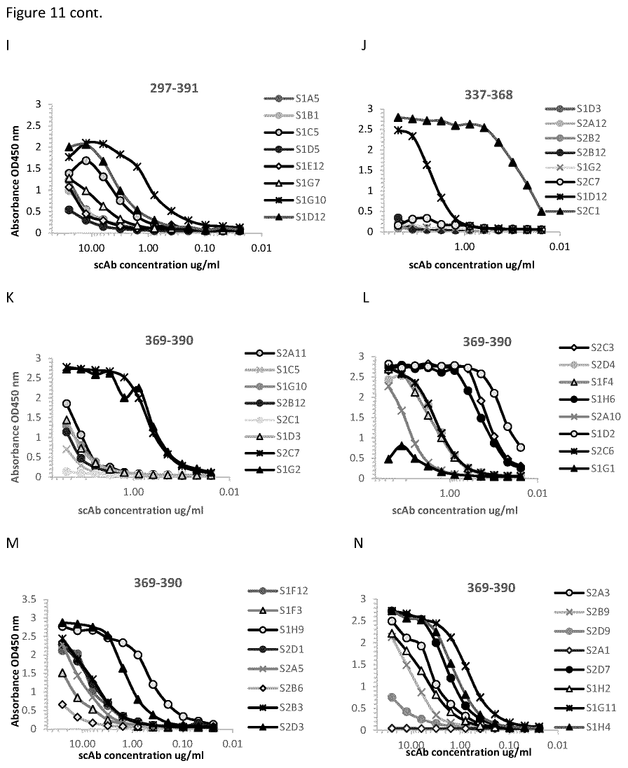

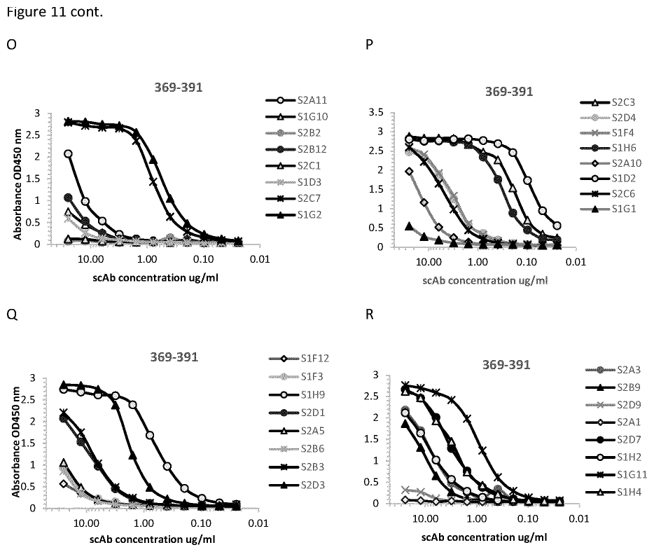

FIG. 11. ELISA based characterisation of the cross-reactivity of ‘S’ group scAbs using various short tau fragments numbered according to their corresponding amino acid residues on hT40 molecule. (A) 186-350, (B) 275-305 (C-D) 266-359 (R1-3), (E-I) 297-391, (J) 360-378 (K-N) 369-391, (O-R) 369-390. A summary of specific ‘S’ scAb binding to these shorter antigens are shown in Table 16

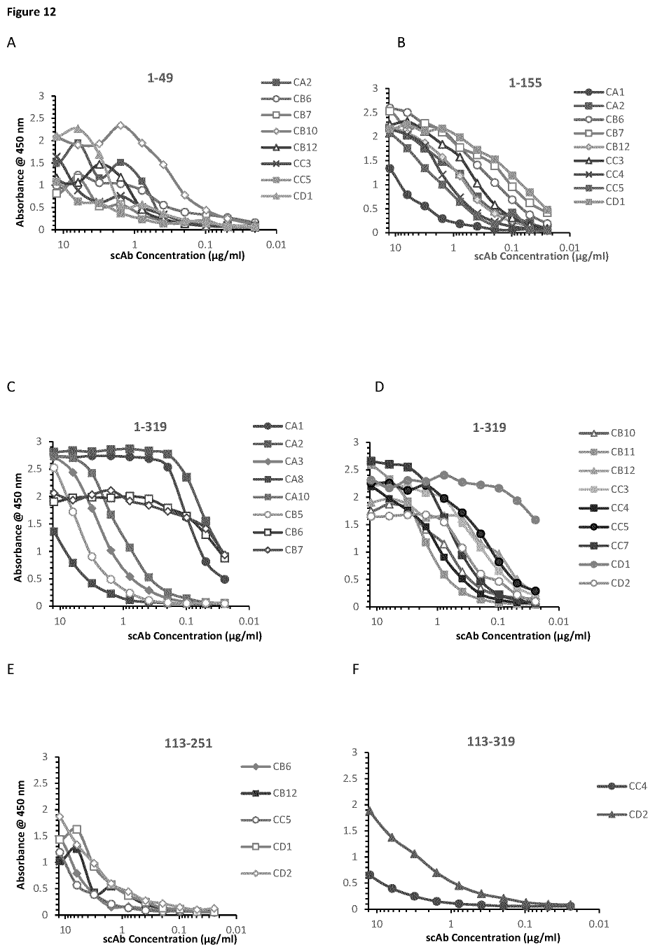

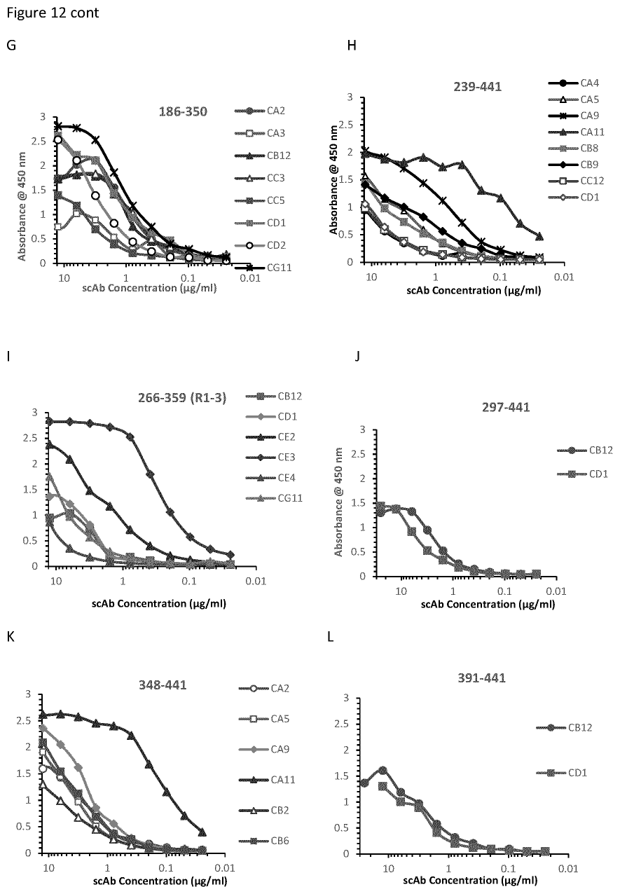

FIG. 12. ELISA based characterisation of the cross-reactivity of ‘C’ group scAbs using various short tau fragments numbered according to their corresponding amino acid residues on hT40 molecule. (A) 1-49, (B) 1-155 (C-D) 1-319, (E) 113-251 (F) 113-319, (G) 186-350 (H) 239-441 (I) 266-359 (R1-3), (J) 297-441, (K) 348-441, (L) 391-441. A summary of specific ‘C’ scAb binding to these shorter antigens are shown in Table 17

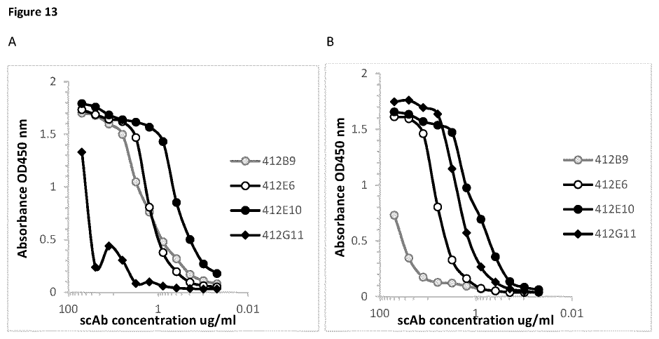

FIG. 13. Cross-reactivity of ‘412’ group scAbs to hT40. (A) showing binding of scAbs to biotinylated 412-441 peptide which was used as the antigen for the selection of C terminal binders. (B) binding profiles of four scAbs which were shown to be cross-reactive in hT40 binding ELISA.

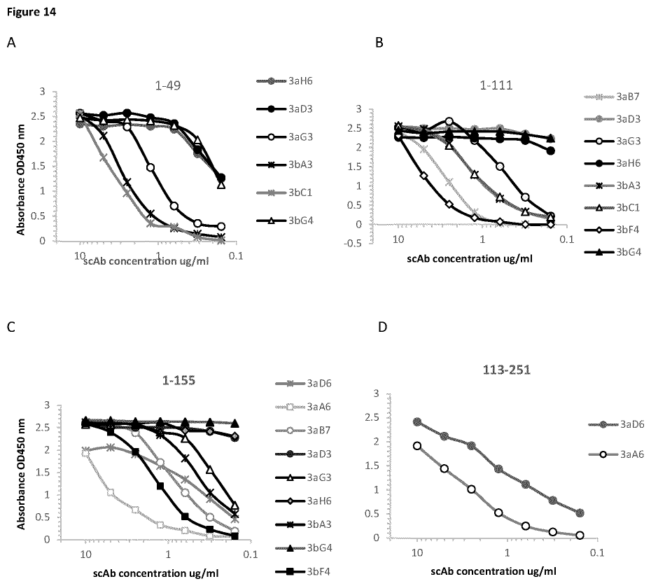

FIG. 14. ELISA based characterisation of the cross-reactivity of ‘3a’ & ‘3b’ group scAbs using various short tau fragments numbered according to their corresponding amino acid residues on hT40 molecule. (A) 1-49, (B) 1-111 (C) 1-155, (D) 113-251. A summary of specific ‘3a’ & ‘3b’ group scAbs binding to these shorter antigens are shown in Table 18

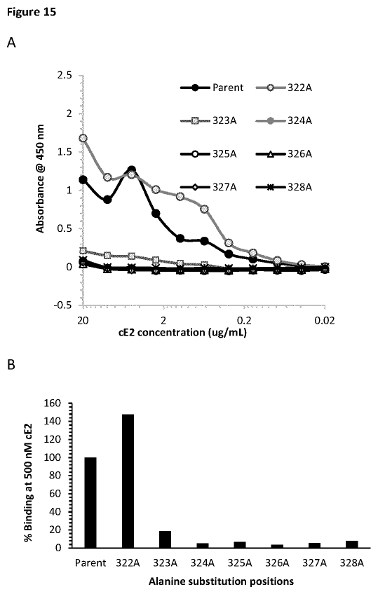

FIG. 15. (A) Immunoreactivity of CE2 scAb to the parent peptide and a series of alanine substituted residues at positions indicated in table 19. (B) Percentage binding of 500 nM scAb to each of these ASM peptides with respect to the parent peptide.

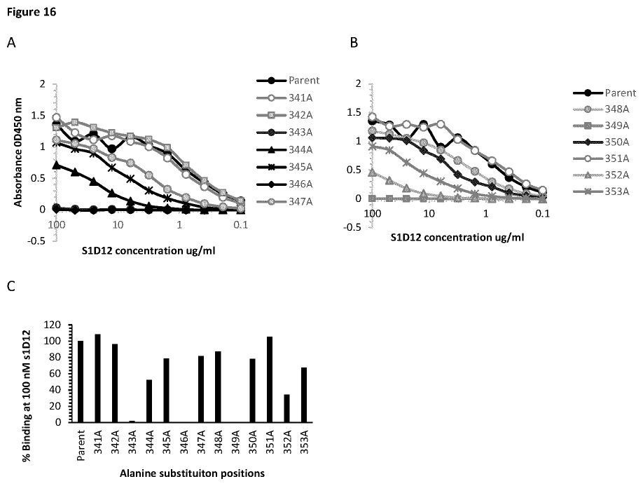

FIG. 16. (A-B) Immunoreactivity of S1D12 scAb to the parent peptide and a series of alanine substituted residues at positions indicated in table 20. (C) Percentage binding of 500 nM scAb to each of these ASM peptides with respect to the parent peptide.

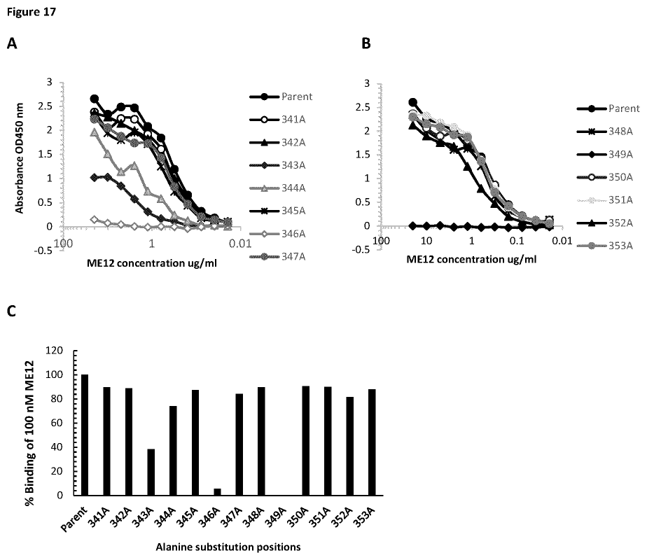

FIG. 17. (A-B) Immunoreactivity of ME12 scAb to the parent peptide and a series of alanine substituted residues at positions indicated in table 20. (C) Percentage binding of 100 nM scAb to each of these ASM peptides with respect to the parent peptide.

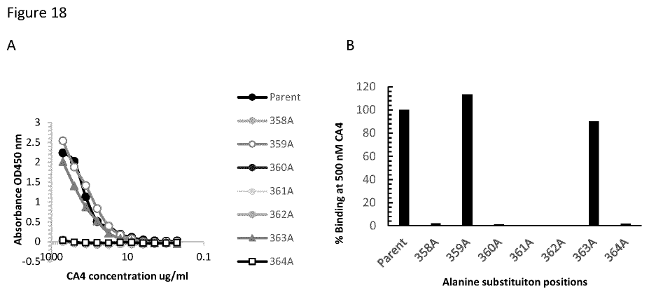

FIG. 18. (A) Immunoreactivity of CA4 scAb to the parent peptide and a series of alanine substituted residues at positions indicated in table 21. (B) Percentage binding of 500 nM scAb to each of these ASM peptides with respect to the parent peptide.

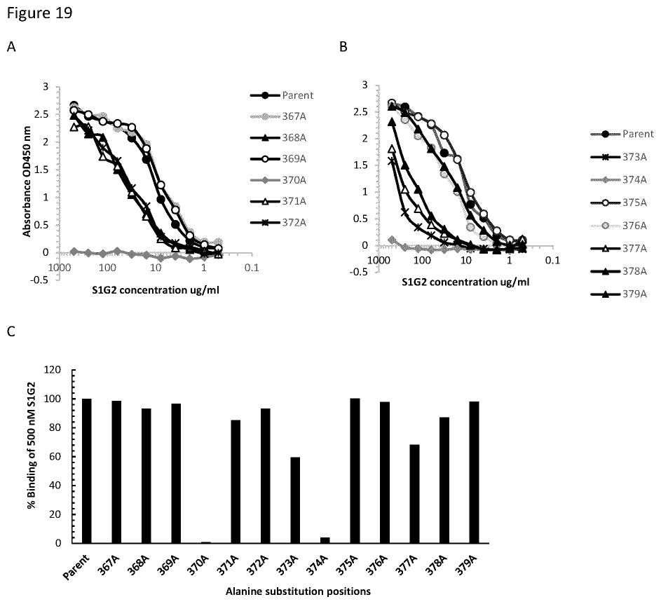

FIG. 19. (A-B) Immunoreactivity of S1G2 scAb to the parent peptide and a series of alanine substituted residues at positions indicated in table 22. (C) Percentage binding of 500 nM scAb to each of these ASM peptides with respect to the parent peptide.

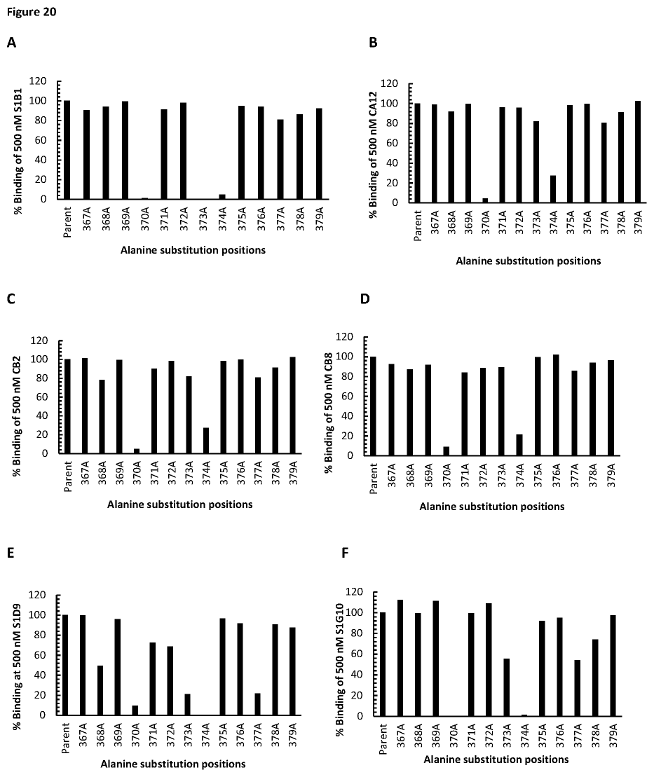

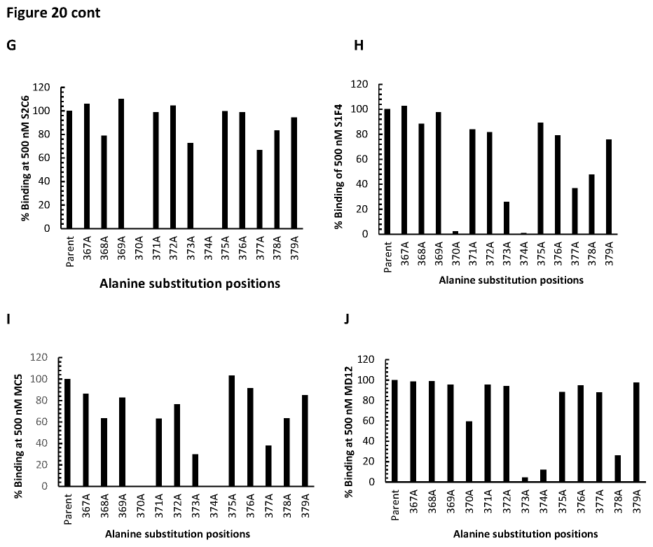

FIG. 20. Percentage binding of various 367-379 region scAbs to ASM peptides with respect to the parent peptide. The scAbs tested included (A) S1B1, (B) CA12, (C) CB2, (D) CB8, (E) S1D9, (F) S1G10, (G) S2C6, (H) S1F4, (I) MC5, (J) MD12. The critical binding residues of these scAbs are similar to the representative clone S1G2, where alanine substitution in positions 370, 373, 374, 377 or 378 resulted in reduction in antibody binding.

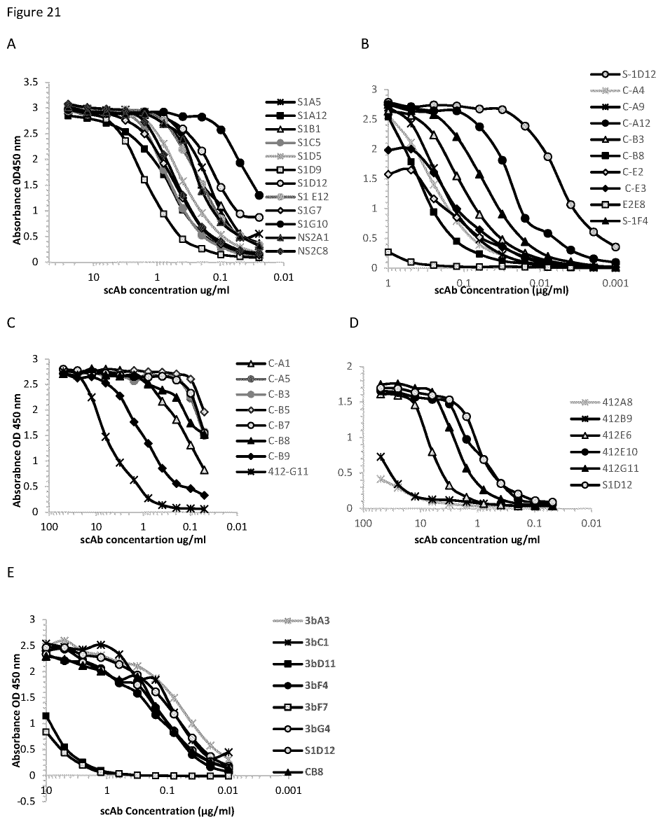

FIG. 21. Ranking of the binding affinities of anti-Tau scAbs using hT40. scAbs with known kD values such as NS2A1 and S1D12 were used to rank the relative binding affinities of test scAbs and those with similar binding profiles were shortlisted and selected for Biacore analysis (A) ‘S’ group clones, (B-C) ‘C’ clones, (D) ‘412’ clones (E) ‘3a’ clones

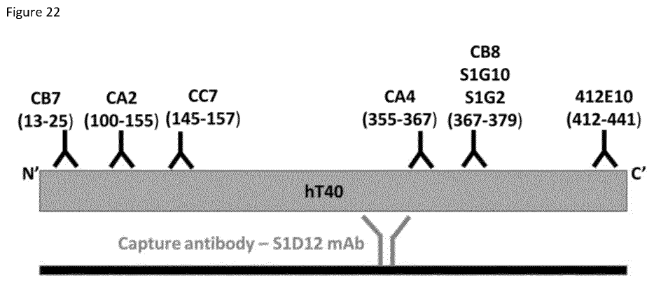

FIG. 22. Schematic representation of the sandwich ELISA format for calculating the LoDs of various antibody pairs.

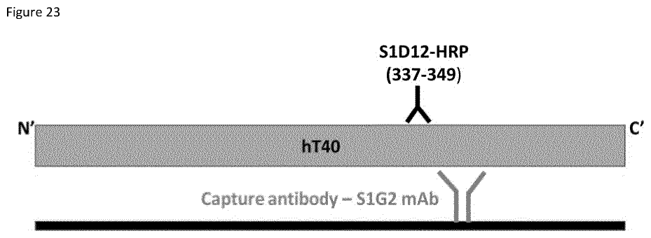

FIG. 23. Schematic representation of the sandwich ELISA format for calculating the LoDs using S1G2 mAb as the capture antibody and HRP conjugated S1D12 mAb for detection

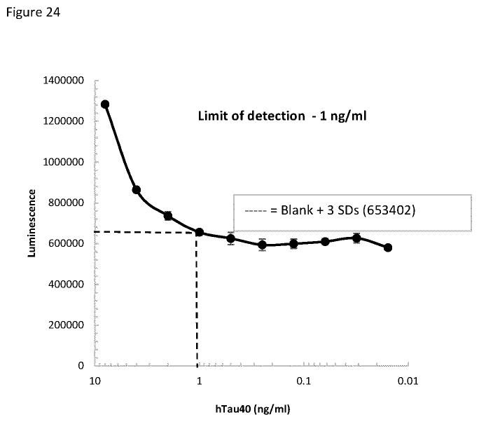

FIG. 24. Sandwich ELISA graph showing the LoD achieved using S1G2 mAb as the capture antibody and HRP labelled S1D12 mAb detection. Antibody binding was measured using chemiluminescence and the LOD for hT40 is ˜1 ng/ml for this assay set up.

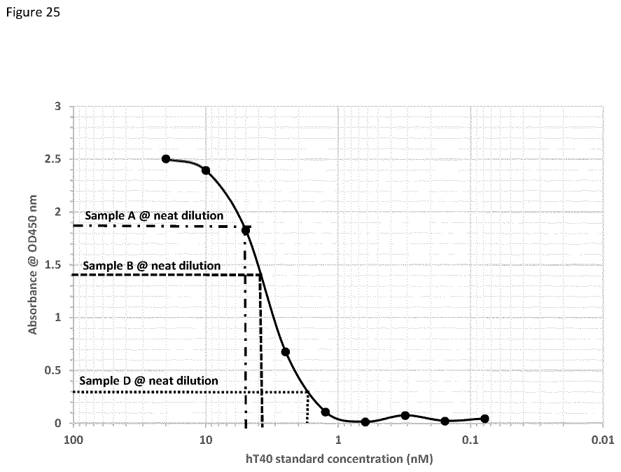

FIG. 25. ELISA #1 hT40 standard curve generated using S1D12 mAb capture and CB7 scAb detection. Concentrations of the four spiked samples-Sample A, B, C and D were determined by plotting their respective absorbance values on this standard curve. Sample C did not generate a binding signal and therefore confirmed the absence of any tau species with N terminal region in this mix. Concentrations and types of tau species deduced from this assay is given in table 29.

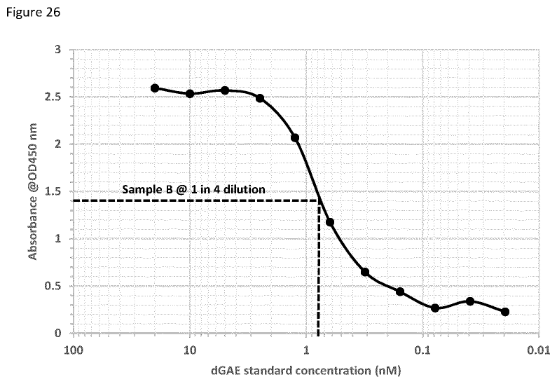

FIG. 26. ELISA #2 dGAE standard curve generated using S1D12 mAb capture and E2E8 scAb detection. Concentrations of the four spiked samples-Sample A, B, C and D were determined by plotting their respective absorbance values on this standard curve. Samples A, C and D did not generate any binding signals and therefore confirmed the absence of dGAE species within these mixes. Concentrations and types of tau species deduced from this assay is given in table 29.

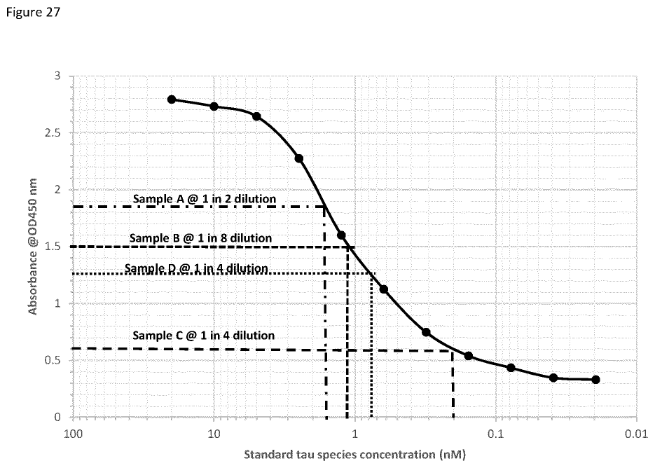

FIG. 27. ELISA #3 Average standard curve generated using S1D12 mAb capture and S1G2 scAb detection. Concentrations of the four spiked samples-Sample A, B, C and D were determined by plotting their respective absorbance values on this standard curve. Concentrations and types of tau species deduced from this assay is given in table 29.

FIG. 28. Comparison of the binding profiles of various SDS (+/−Triton X-100) treated dGAE monomer or aggregates in a sandwich ELISA system. S1D12 mAb was used as the capture antibody and S1G2 as the detection scAb. Here the effect of SDS+Triton X-100 in restoring the immunoreactivity of is noticed. This mAb-scAb pairing can detect approximately 2 ng/ml dGAE aggregates in a simple sandwich ELISA.

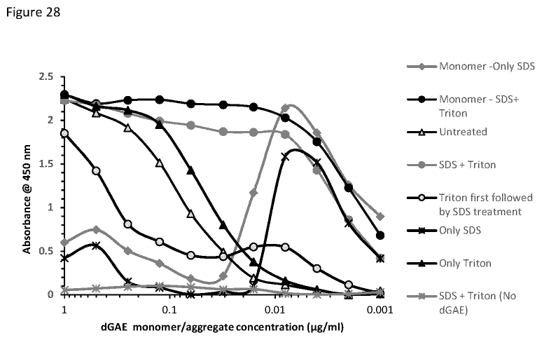

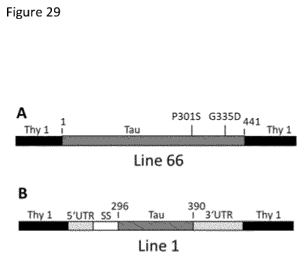

FIG. 29. A) L66 cDNA containing human tau (hT40) and the point mutations P301S and G335D (2N4R Tau, 441 amino acids) B) L1 cDNA codes for human tau amino acid residues 296-390 with a signal sequence and murine Thy1 expression sequences as described in Melis et al., 2015

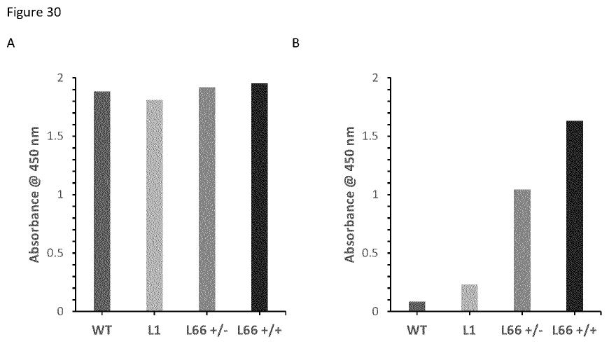

FIG. 30. (A) Detection of tau protein in 50 μg brain homogenate isolated from WT, L1, L66+/− and L66+/+ mice using S1D12 mAb capture and S-1G2 scAb detection. All four samples have similar tau levels when detected using a core region specific antibody pairing (B) Detection of tau protein in 50 μg brain homogenate isolated from WT, L1, L66+/− and L66+/+ mice using S1D12mAb capture and CB7 scAb detection. N′ terminally directed CB7 scAb can specifically detect human tau in Line66 homozygous and heterozygous samples and able to differentiate levels of expression between the two groups.

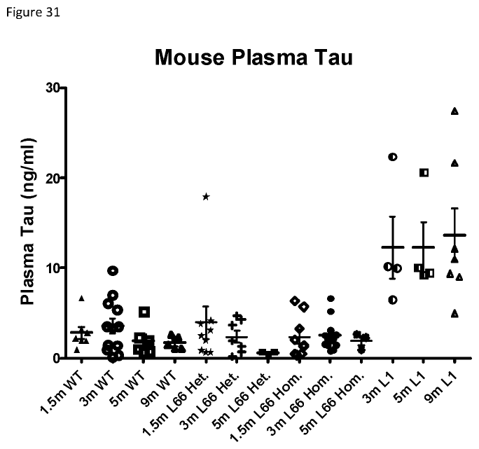

FIG. 31. Plasma tau levels in WT (5 month: 1.947 ng/ml), (9 month: 2.177 ng/ml); L66 (Both 5 month) (+/−: 0.567 ng/ml), (+/+: 1.937 ng/ml); and L1 (5 month: 12.355 ng/ml) (9 month 13.661 ng/ml). Data collected using S1D12 mAb capture and S1G2 scAb detection. Tau species concentrations were determined using standard curves of hT40 for WT and L66 and dGA (296-390) for L1.

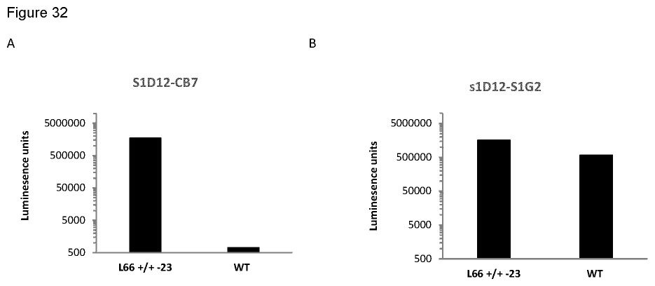

FIG. 32. Detection of plasma tau levels in Line66+/+ mouse sample no: 23 at 1.5 months and comparison with age matched wild type mouse plasma using two different sandwich ELISA pairing. (A) Shows the chemiluminescent signal readings for Line66+/+ and wildtype mice using S1D12 mAb capture and CB7 scAb detection. (b) the signal readings for the same samples using S1D12 mAb capture and S1G2 scAb detection. Line66+/+ mouse shows at least 1000-fold increase in signal intensity compared to the wild type when using S1D12 mAb-CB7 scAb pairing which specifically detects N terminal hT40 in this sample.

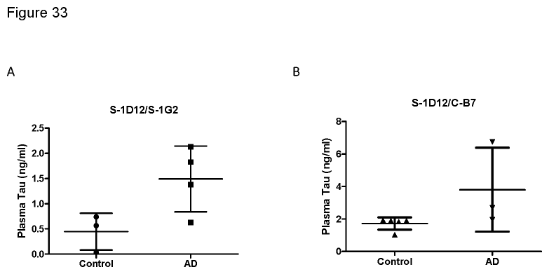

FIG. 33. Plasma tau levels in AD samples vs age matched controls using S1D12-S1G12 (core region) and S1D12-CB7 (N terminal) detection pairs.

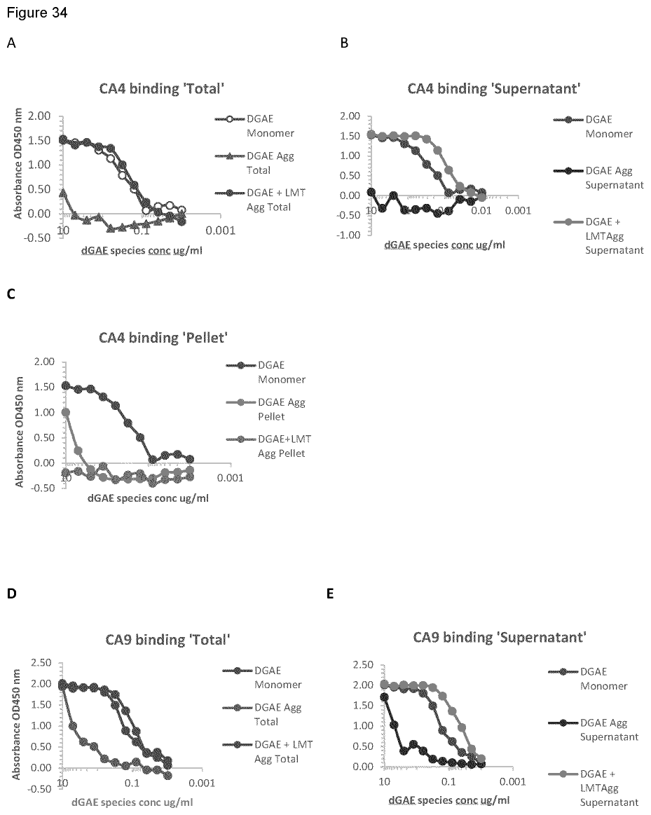

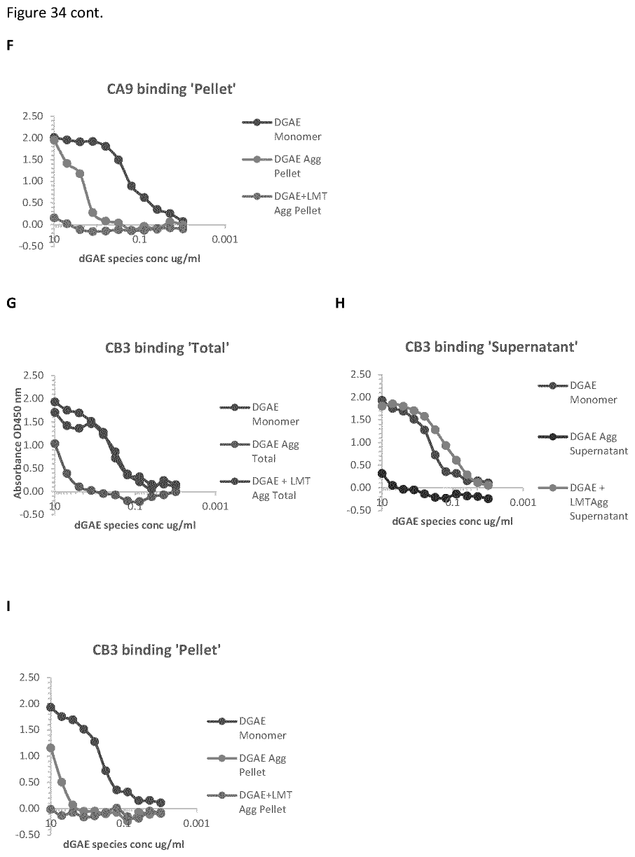

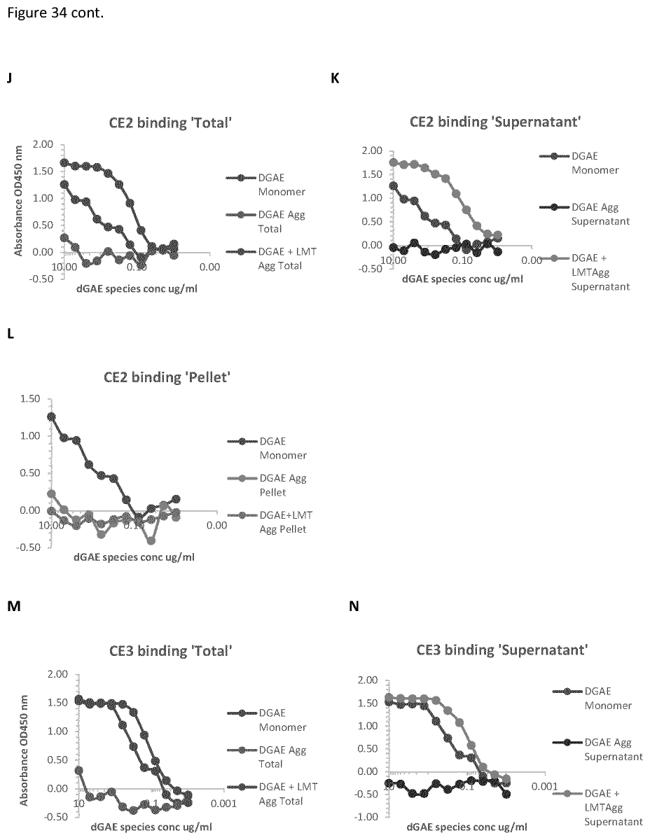

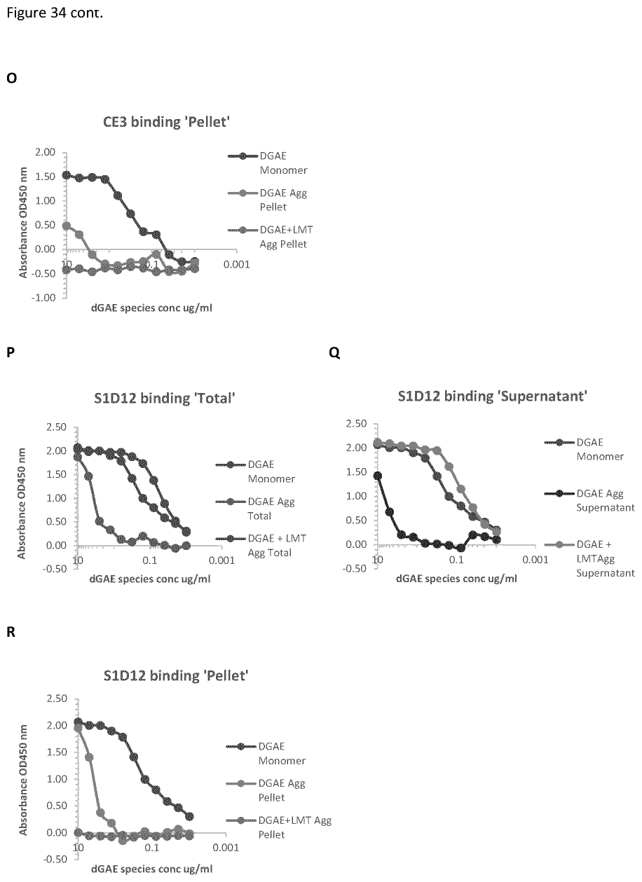

FIG. 34. Sandwich ELISA graphs showing the increase in immunoreactivity of core region scAbs to dGAE ‘total’, ‘supernatant’ and ‘pellet’ aggregation inhibition samples prepared in the presence of LMTM. dGAE monomer was included as assay control to indicate the binding profiles of each test scAbs to their corresponding epitopes in non-aggregated samples. (A-C) CA4 scAb, (D-F) CA9, (G-I) CB3 scAb, (J-L) CE2 scAb, (M-O) CE3, (P-R) S1D12 scAb. Lack of antibody binding in some dGAE+LMTM pellet samples corresponds to the absence protein present in this group as confirmed by SDS gel (data not included)

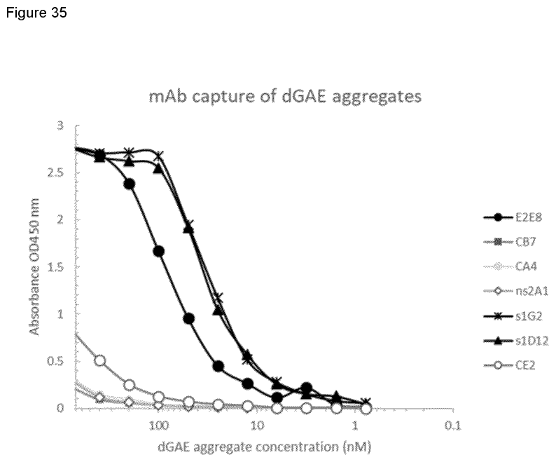

FIG. 35. mAb capture of dGAE aggregates. Various antibodies, as indicated, were coated on solid-phase ELISA and used to capture aggregates of dGAE. Captured dGAE was detected using S1G2 scAb for all capture antibodies except S1G2 mAb. For S1G2 mAb capture, S1D12 scAb was used as the detector antibody.

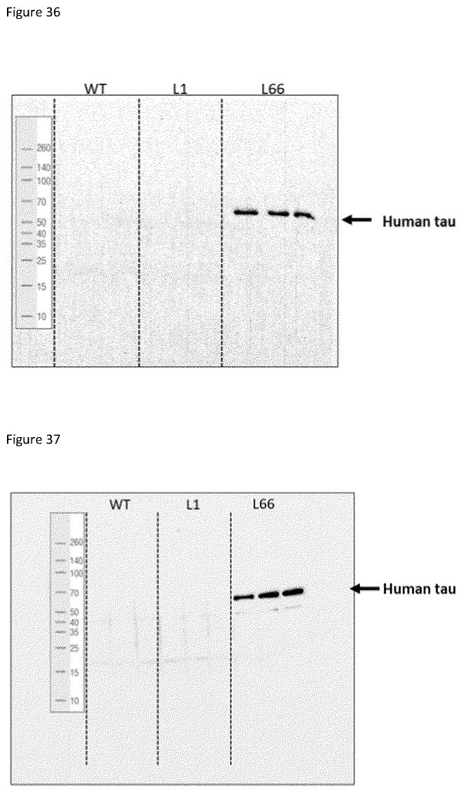

FIG. 36. Western blot showing brain-derived tau labelled with a human-specific CB7 antibody which binds an N-terminal epitope (residues 13-25) absent in mouse tau. Bands are present in lanes containing 20 μg protein homogenate from 5-month-old L66^{+/+} mouse brain but not in the lanes containing samples from either WT or L1^{+/+} brains. The protein ladder superimposed on the left of the blot provides an approximation of the relative size of proteins on the gel, but it is known that the apparent size of tau is considerably greater than the actual molecular mass.

FIG. 37. Western blot showing tau labelled with human-specific CC7 antibody that recognises an epitope within residues 145-157. Human-specific tau is only detected in L66^{+/+} mouse brain and not in samples from either WT or L1^{+/+} The protein ladder is as described in FIG. 36.

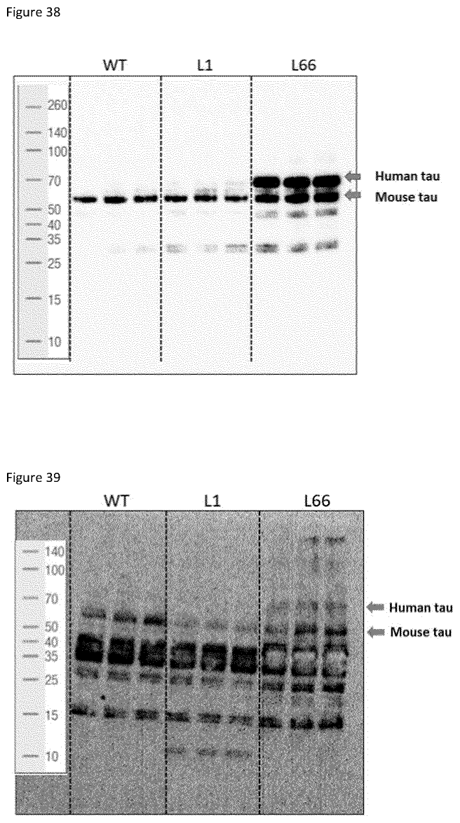

FIG. 38 Western blot labelled with S1D12 tau core antibody. Bands are present in lanes containing 20 μg protein homogenate from 5-month-old L66^{+/+}, L1 and WT mice brains. Mouse tau (indicated by the lower arrow) appears as a band of approximately 55 kDa in each of the samples. Human tau (indicated by the upper arrow) appears as a protein at 68 kDa that is present only in the L66+/+ samples. Protein ladder as for FIG. 36.

FIG. 39. Western blot labelled with S1G2 core antibody. Bands are present in lanes containing 20 μg protein homogenate from 5-month-old L66^{+/+}, L1^{+/+} and WT mice brains. Mouse tau (indicated by the lower arrow) appears as a band at about 55 kDa in each of the samples. Human tau (indicated by the upper arrow) appears at about 68 kDa but only in the L66^{+/+} samples. Using this antibody, a band at around 10 kDa is visible in the L1^{+/+} samples. Protein ladder as for FIG. 36.

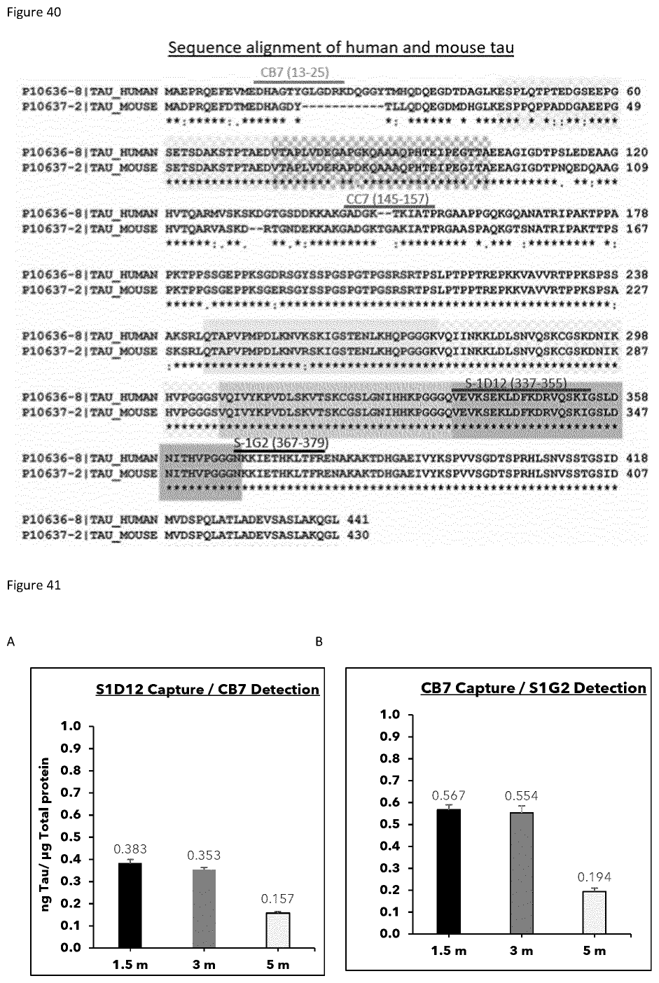

FIG. 40. Sequence comparison of human and mouse tau. The sequences shown consist of SEQ ID No. 1 for human tau (two gaps introduced to allow sequence alignment) and SEQ ID NO: 589 for mouse tau. The protein regions that contain the epitopes of candidate antibodies are superimposed. Both CB7 and CC7 binding regions in human tau are not present in mouse tau. In contrast, protein regions containing the epitopes for the antibodies S1D12 and S1G2 show 100% homology between the 2 species.

FIG. 41. A) Paired antibody ELISAs with S1D12 capture and CB7 detection show a progressive decrease in signal with advancing age in brain homogenate samples from L66^{+/+}mice in. B) When reversing the orientation of the assay, and using CB7 as the capture along with S1G2 as detector for brain homogenate samples, a similar pattern of decreasing signal with age is observed.

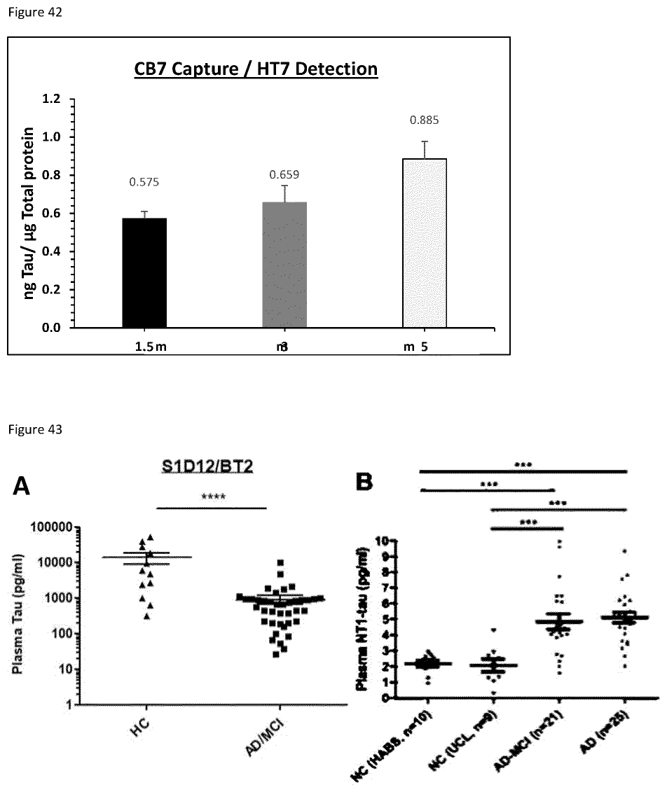

FIG. 42. Paired antibody ELISA with CB7 capture and HT7 detection shows a progressive increase in signal with advancing age for L66^{+/+} mice. This suggests an accumulation of small N-terminally intact fragments created by truncation between the core region and the N-terminal region of tau protein or protein fragments.

FIG. 43. (A) Plasma tau levels in healthy control (HC) and patients with a confirmed diagnosis of Alzheimer's disease or mild cognitive impairment (AD/MCI). The concentration of the core-proline region measured using S1D12 capture beads paired with BT2 as detector is significantly higher in healthy control than in AD/MCI samples. A total of 12 heathy control plasma samples and 42 AD/MCI samples were analysed using the Simoa® assay. **** p<0.0001 (B) NT1 assay data (Chen et al 2019) reported detecting slightly increased levels of NT-1 plasma tau in AD-MCI (AD biomarker positive-mild cognitive impairment) and AD (AD biomarker positive-clinical AD) patients compared to NC (normal control) using Tau12-BT2 antibodies.

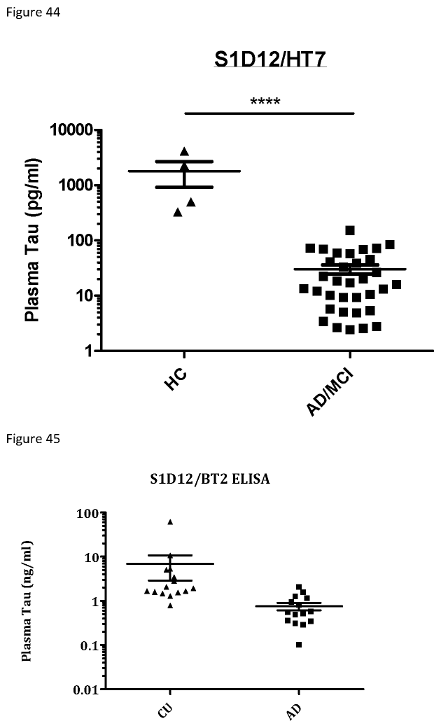

FIG. 44. Plasma tau levels in healthy control (HC) and patients with a confirmed diagnosis of Alzheimer's disease or mild cognitive impairment (AD/MCI). The concentration of the core-proline region measured using S1D12 capture beads paired with HT7 detector is significantly higher in healthy control than in AD/MCI samples. A total of 4 heathy control plasma samples and 34 AD/MCI samples were analysed using the Simoa® assay. **** p<0.0001

FIG. 45. S1D12 (capture)/BT2 (detector) plasma tau measured by chemiluminescent ELISA, AD-samples from subjects with a confirmed clinical diagnosis of AD; CU-samples from age-matched, cognitively-unimpaired subjects.

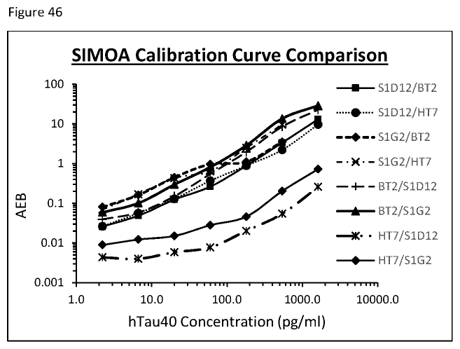

FIG. 46. Simoa® calibrator curves generated for various antibody combination assays used in human plasma experiments.

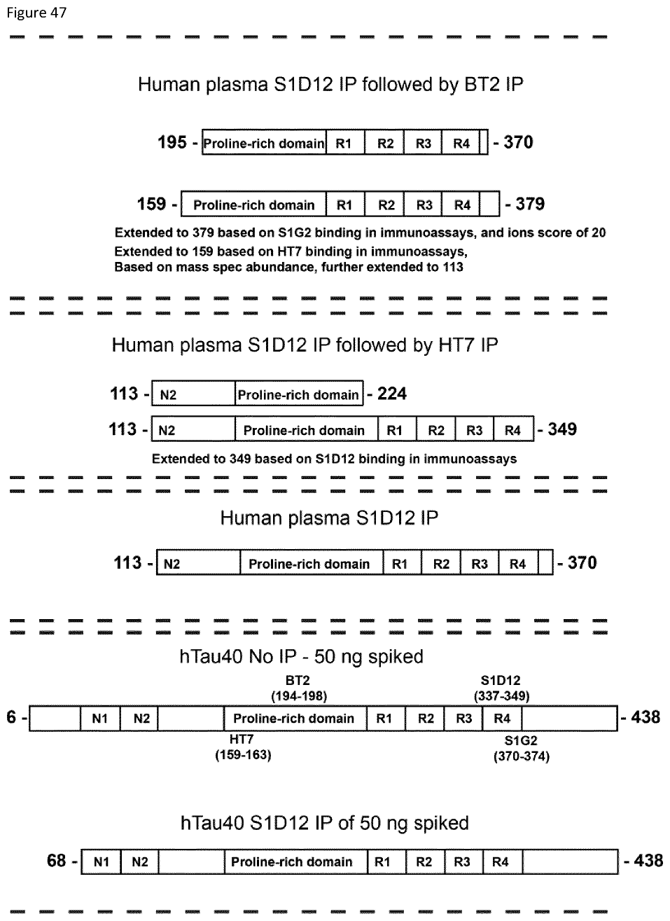

FIG. 47. Immunoprecipitation and tryptic digestion LC-MS reveal core region containing tau fragments in human plasma. Tau fragments detected by abundance from the human plasma sample following immunoprecipitation (IP) and LC-MS analysis. Fragments detected from a sample spiked with htau40, without immunoprecipitation, are also given for comparison.

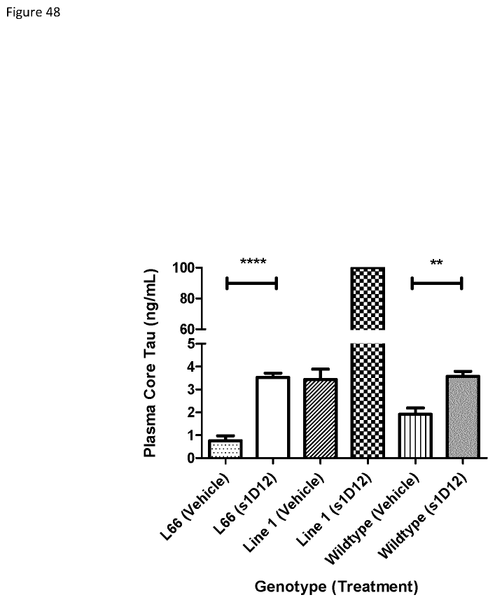

FIG. 48. Core tau levels in mouse plasma are increased by S1D12 mAb treatment. Using CA4 (355-367) and S1G2 (367-379) antibodies, core region tau levels were seen to be profoundly elevated in L66 mice treated with S1D12 compared to the vehicle group in L1 (more than 100-fold increase). Individual mouse samples were analysed in duplicate, and values represent mean concentration of the tau fragment detected by CA4-S1G2 antibody pairing. In L66, more than a threefold increase was achieved in the treatment group, whereas in wild-type mice less than a two-fold increase was observed. n=5 or 6, error bars represent SEM, Unpaired t-test was performed between vehicle and treatment groups, ****P<0.0001; **P<0.01

### DETAILED DESCRIPTION OF THE INVENTION

According to a first aspect, the invention provides an in vitro method for detecting a tau protein or fragment thereof in a saliva sample comprising contacting the sample with a first specific binding molecule wherein the first specific binding molecule binds to an epitope within residues 297 to 391 of SEQ ID NO: 1.

All residue numbers of the Tau protein sequence and structure in the present disclosure refer to the residues of SEQ ID NO:1, which is the sequence of the four repeat isoform 2N4R of human Tau protein (Uniprot ID P10636-8), or homologous positions in other species or variants thereof. Human Tau isoform 2N4R (Uniprot ID P10636-8) corresponds to amino acids 1-124, 376-394 and 461-758 of full length Tau, Uniprot ID P10636 or P10636-1, provided as SEQ ID NO:2. SEQ ID NO: 2 relates to a longer form of Tau found in the peripheral nervous system (PNS) but not the central nervous system (CNS). As used herein, references to “full-length” tau refer to SEQ ID NO: 1 (the relevant sequence for the CNS) and not to SEQ ID NO: 2 (which is not relevant in the CNS).

SEQ ID NO: 1 (Isoform Tau-F, also known as Tau-4,
2N4R, 441 amino acids):
>sp|P10636-8|TAU_HUMAN Isoform Tau-F of Micro-
tubule-associated protein tau OS = Homo sapiens
OX = 9606 GN = MAPT
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNVKSKIGSTENLKHQPGGGKVQIINKKLDLSNVQSKCGSKDNIKHV
PGGGSVQIVYKPVDLSKVTSKCGSLGNIHHKPGGGQVEVKSEKLDFKDRV
QSKIGSLDNITHVPGGGNKKIETHKLTFRENAKAKTDHGAEIVYKSPVVS
GDTSPRHLSNVSSTGSIDMVDSPQLATLADEVSASLAKQGL
SEQ ID NO: 2 (Full length human Tau, Isoform PNS-
Tau, 758 amino acids);
>sp|P10636-1|TAU_HUMAN Microtubule-associated
protein tau OS = Homo sapiens OX = 9606 GN = MAPT
PE = 1 SV = 5
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQEPESGKVVQEGFLREPGPPGLSHQLM
SGMPGAPLLPEGPREATRQPSGTGPEDTEGGRHAPELLKHQLLGDLHQEG
PPLKGAGGKERPGSKEEVDEDRDVDESSPQDSPPSKASPAQDGRPPQTAA
REATSIPGFPAEGAIPLPVDFLSKVSTEIPASEPDGPSVGRAKGQDAPLE
FTFHVEITPNVQKEQAHSEEHLGRAAFPGAPGEGPEARGPSLGEDTKEAD
LPEPSEKQPAAAPRGKPVSRVPQLKARMVSKSKDGTGSDDKKAKTSTRSS
AKTLKNRPCLSPKHPTPGSSDPLIQPSSPAVCPEPPSSPKYVSSVTSRTG
SSGAKEMKLKGADGKTKIATPRGAAPPGQKGQANATRIPAKTPPAPKTPP
SSGEPPKSGDRSGYSSPGSPGTPGSRSRTPSLPTPPTREPKKVAVVRTPP
KSPSSAKSRLQTAPVPMPDLKNVKSKIGSTENLKHQPGGGKVQIINKKLD
LSNVQSKCGSKDNIKHVPGGGSVQIVYKPVDLSKVTSKCGSLGNIHHKPG
GGQVEVKSEKLDFKDRVQSKIGSLDNITHVPGGGNKKIETHKLTFRENAK
AKTDHGAEIVYKSPVVSGDTSPRHLSNVSSTGSIDMVDSPQLATLADEVS
ASLAKQGL

As used herein “mouse tau” refers to Isoform Tau-A which has the sequence of Uniprot ID P10637-2, provided as SEQ ID NO: 589:

MADPRQEFDTMEDHAGDYTLLQDQEGDMDHGLKESPPQPPADDGAEEPGS
ETSDAKSTPTAEDVTAPLVDERAPDKQAAAQPHTEIPEGITAEEAGIGDT
PNQEDQAAGHVTQARVASKDRTGNDEKKAKGADGKTGAKIATPRGAASPA
QKGTSNATRIPAKTTPSPKTPPGSGEPPKSGERSGYSSPGSPGTPGSRSR
TPSLPTPPTREPKKVAVVRTPPKSPSASKSRLQTAPVPMPDLKNVRSKIG
STENLKHQPGGGKVQIINKKLDLSNVQSKCGSKDNIKHVPGGGSVQIVYK
PVDLSKVTSKCGSLGNIHHKPGGGQVEVKSEKLDFKDRVQSKIGSLDNIT
HVPGGGNKKIETHKLTFRENAKAKTDHGAEIVYKSPVVSGDTSPRHLSNV
SSTGSIDMVDSPQLATLADEVSASLAKQGL

dGAE97 refers to the 97 residues fragment of Tau (2N4R) with N-terminus at residue Asp-295 and C-terminus at residue Glu-391, as described in SEQ ID NO: 3, or at homologous positions in other species (the residues mentioned referring to the human or mouse Tau sequence, which are identical in this region). As will be apparent to the skilled person, dGAE97 also corresponds to the fragment of Isoform PNS-Tau (P10636-1) with N-ter at Asp-612 and C-ter at Glu-708.

SEQ ID NO: 3 (dGAE97, human/mouse, 97 amino
acids):
DNIKHVPGGGSVQIVYKPVDLSKVTSKCGSLGNIHHKPGGGQVEVKSEK
LDFKDRVQSKIGSLDNITHVPGGGNKKIETHKLTFRENAKAKTDHGAE

dGAE95 refers to the 95 residues fragment of Tau (2N4R) with N-terminus at residue Ile-297 and C-terminus at residue Glu-391, as described in SEQ ID NO: 4, or at homologous positions in other species (the residues mentioned referring to the human or mouse Tau sequence, which are identical in this region). As will be apparent to the skilled person, dGAE95 also corresponds to the fragment of Isoform PNS-Tau (P10636-1) with N-ter at Ile-614 and C-ter at Glu-708. This sequence may sometimes be referred to simply as “dGAE”. Residues 297 to 391 of Tau (2N4R) are also known as the predominant fragment isolated from proteolytically stable core of the paired helical filament (PHF). References herein to “residues 297 to 391 of SEQ ID NO: 1” may therefore be substituted for references to SEQ ID NO: 4.

SEQ ID NO: 4 (dGAE95 or “dGAE”, human/mouse, 95
amino acids):
IKHVPGGGSVQIVYKPVDLSKVTSKCGSLGNIHHKPGGGQVEVKSEKLD
FKDRVQSKIGSLDNITHVPGGGNKKIETHKLTFRENAKAKTDHGAE

“dGA” refers to the 94 residues fragment of Tau (2N4R) with N-terminus at residue Ile-297 and C-terminus at residue Ala-390, as described in SEQ ID NO: 5, or at homologous positions in other species (the residues mentioned referring to the human or mouse Tau sequence, which are identical in this region).

SEQ ID NO: 5 (dGA, human/mouse, 94 amino acids):
IKHVPGGGSVQIVYKPVDLSKVTSKCGSLGNIHHKPGGGQVEVKSEKLD
FKDRVQSKIGSLDNITHVPGGGNKKIETHKLTFRENAKAKTDHGA

dGAE73 refers to the fragment of Tau (2N4R) with N-terminus at residue Val-306 and C-terminus at residue Phe-378, as described in SEQ ID NO: 6, or at homologous positions in other species (the residues mentioned referring to the human or mouse Tau sequence, which are identical in this region). This fragment corresponds to residues 306-378 of the sequence identified by cryo-EM as being the core of PHFs isolated from AD brain tissue (Fitzpatrick et al, 2017; Nature). The core can extend beyond these residues but is limited by the resolution of the cryo-EM. As will be apparent to the skilled person, dGAE73 also corresponds to the fragment of Isoform PNS-Tau (P10636-1) with N-ter at Val-623 and C-ter at Phe-695.

SEQ ID NO: 6 (dGAE73, human/mouse, 73 amino
acids):
VQIVYKPVDLSKVTSKCGSLGNIHHKPGGGQVEVKSEKLDFKDRVQSKI
GSLDNITHVPGGGNKKIETHKLTF

The PHF core refers to residues 296 to 391 of Tau (2N4R) as described in SEQ ID NO: 3, or at homologous positions in other species (the residues mentioned referring to the human or mouse Tau sequence, which are identical in this region).

A further fragment of the PHF core is residues 308 to 378 of Tau (2N4R) with N-terminus at residue Ile-308 and C-terminus at residue Phe-378, as described in SEQ ID NO: 7, or at homologous positions in other species (the residues mentioned referring to the human or mouse Tau sequence, which are identical in this region).

SEQ ID NO: 7 (dGAE71, residues 308 to 378 of
2N4R, human/mouse, 71 amino acids):
IVYKPVDLSKVTSKCGSLGNIHHKPGGGQVEVKSEKLDFKDRVQSKIGS
LDNITHVPGGGNKKIETHKLTF

### First Specific Binding Molecule

The method comprises contacting the sample with a first specific binding molecule wherein the first specific binding molecule binds to an epitope within residues 297 to 391 of SEQ ID NO: 1. The first specific binding molecule may be any specific binding molecule disclosed herein that binds to an epitope within residues 297 to 391 of SEQ ID NO: 1, preferably within residues 307 to 391 of SEQ ID NO: 1, more preferably within residues 337 to 379 of SEQ ID NO: 1. The following describes the first specific binding molecule. Any references under this sub-heading to a or the specific binding molecule are to the first specific binding molecule.

The epitope of the first specific binding molecule may be within SEQ ID NO: 4, SEQ ID NO: 5, SEQ ID NO: 6 or SEQ ID NO: 7.

The epitope of the specific binding molecule may be within residues 297 to 391 of SEQ ID NO: 1. Residues 297 to 391 of full-length Tau are also known as the predominant fragment isolated from proteolytically stable core of the paired helical filament (PHF) or the PHF-core fragment. Therefore, the epitope of the specific binding molecule may be within the PHF-core or within the dGAE fragment. Accordingly, the epitope of the specific binding molecule may be within SEQ ID NO 4.

The epitope of the specific binding molecule may be within residues 297 to 390 of SEQ ID NO: 1. Residues 297 to 390 of full-length Tau are also known as the dGA fragment. Therefore, the epitope of the specific binding molecule may be within the dGA fragment. Accordingly, the epitope of the specific binding molecule may be within SEQ ID NO: 5. The epitope of the specific binding molecule may be within dGAE73 and/or dGAE71. Accordingly, the epitope of the specific binding molecule may be within SEQ ID NO: 6 and/or SEQ ID NO: 7.

The epitope of the specific binding molecule may be within residues 308 to 378 of SEQ ID NO: 1. Residues 308 to 378 of full-length Tau are also known as the PHF core. Therefore, the epitope of the specific binding molecule may be within the PHF core. Accordingly, the epitope of the specific binding molecule may be within SEQ ID NO: 7.

Typically, a specific binding molecule binds to a polypeptide or protein molecule comprising its epitope. Therefore, the specific binding molecule may bind to SEQ ID NO: 1 or a fragment thereof comprising residues 297 to 391 of SEQ ID NO: 1. The specific molecule may bind to SEQ ID NO: 3, SEQ ID NO: 4, SEQ ID NO: 5, SEQ ID NO: 6 and/or SEQ ID NO: 7. The specific molecule may bind to the PHF or the dGAE fragment. the specific binding molecule may bind to the dGA fragment. The specific binding molecule may bind to the PHF core. The specific binding molecule may bind to a polypeptide or protein molecule comprising an amino acid sequence selected from the group consisting of residues 337 to 355 of SEQ ID NO: 1, residues 367 to 379 of SEQ ID NO: 1, residues 331 to 360 of SEQ ID NO: 1, residues 355 to 367 of SEQ ID NO: 1, residues 379 to 391 of SEQ ID NO: 1, residues 297 to 390 of SEQ ID NO: 1, residues 369 to 390 of SEQ ID NO: 1, residues 337 to 368 of SEQ ID NO: 1, residues 1 to 319 of SEQ ID NO: 1, residues 186 to 350 of SEQ ID NO: 1, residues 239 to 348 of SEQ ID NO: 1, residues 266 to 359 of SEQ ID NO: 1, residues 277 to 319 of SEQ ID NO: 1, residues 319 to 331 of SEQ ID NO: 1, residues 348 to 390 of SEQ ID NO: 1, residues 348 to 441 of SEQ ID NO: 1, residues 359 to 391 of SEQ ID NO: 1, and residues 360 to 390 of SEQ ID NO: 1.

The first specific binding molecule may bind to an epitope within residues 307 to 391 of SEQ ID NO: 1. The first specific binding molecule may bind to an epitope within residues 337 to 379 of SEQ ID NO: 1. The first specific binding molecule may bind to an epitope consisting of residues 337 to 349 of SEQ ID NO: 1. The first specific binding molecule may bind to an epitope consisting of residues 337 to 355 of SEQ ID NO: 1. This epitope may be bound by the CDRs of the specific binding molecule referred to as “S1D12” herein.

The first specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 16 (NNAVG);
VHCDR2 comprises the sequence set forth in SEQ ID NO: 18 (GCSSDGTCYYNSALKS);
VHCDR3 comprises the sequence set forth in SEQ ID NO: 21 (GHYSIYGYDYLGTIDY);
VLCDR1 comprises the sequence set forth in SEQ ID NO: 24 (SGSSSNVGGGNSVG);
VLCDR2 comprises the sequence set forth in SEQ ID NO: 26 (DTNSRPS);
VLCDR3 comprises the sequence set forth in SEQ ID NO: 29 (VTGDSTTHDDL);
or for each CDR sequence, an amino acid sequence with
(i) at least 85% identity thereto, and/or
(ii) one, two, or three amino acid substitutions relative thereto.

The first specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 16 (NNAVG);
VHCDR2 comprises the sequence set forth in SEQ ID NO: 18 (GCSSDGTCYYNSALKS);
VHCDR3 comprises the sequence set forth in SEQ ID NO: 21 (GHYSIYGYDYLGTIDY);
VLCDR1 comprises the sequence set forth in SEQ ID NO: 24 (SGSSSNVGGGNSVG);
VLCDR2 comprises the sequence set forth in SEQ ID NO: 26 (DTNSRPS); and
VLCDR3 comprises the sequence set forth in SEQ ID NO: 29 (VTGDSTTHDDL).

The first specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 435 (QVQLQESGPSLVKPSQTLSLTCTVSGFSLN);
VHFR2 comprises the sequence set forth in SEQ ID NO: 436 (WVRQAPGKVPESLV);
VHFR3 comprises the sequence set forth in SEQ ID NO: 437 (RLDITRDTSKNQISLSLSSVTTDDAAVYYCTR);
VHFR4 comprises the sequence set forth in SEQ ID NO: 438 (WGPGLLVTVSS);
VLFR1 comprises the sequence set forth SEQ in ID NO: 439 (QAVLTQPSSVSGSLGQRVSITC);
VLFR2 comprises the sequence set forth in SEQ ID NO: 440 (WYQHLPGSGLKTIIY);
VLFR3 comprises the sequence set forth in SEQ ID NO: 441 (GVPDRFSGSRSGNTATLTINSLQAEDEGDYYC);
VLFR4 comprises the sequence set forth in SEQ ID NO: 442 (VGSGTRLTVLG);
or for each FR sequence, an amino acid sequence with
(i) at least 50% identity thereto, and/or
(ii) one, two, three, four or five amino acid substitutions relative thereto.

The first specific binding molecule may comprise:
(a) A VH domain comprising the sequence set forth in SEQ ID NO: 443 (QVQLQESGPSLVKPSQTLSLTCTVSGFSLNNNAVGWVRQAPGKVPESLVGCSSDGTCY YNSALKSRLDITRDTSKNQISLSLSSVTTDDAAVYYCTRGHYSIYGYDYLGTIDYWGPGLL VTVSS); and/or
(b) a VL domain comprising the sequence set forth in SEQ ID NO: 444 (QAVLTQPSSVSGSLGQRVSITCSGSSSNVGGGNSVGWYQHLPGSGLKTIIYDTNSRPSG VPDRFSGSRSGNTATLTINSLQAEDEGDYYCVTGDSTTHDDLVGSGTRLTVLG);
or a humanized variant thereof.

The first specific binding molecule may specifically bind to a polypeptide or protein molecule comprising an amino acid sequence comprising residues 337 to 355 of SEQ ID NO: 1 with a K_{D} of less than around 500 pM, optionally wherein the specific binding is measured by surface plasmon resonance (SPR) and optionally wherein
i. The K_{D} for binding to SEQ ID NO: 1 is around 50 pM to around 150 pM, and/or
ii. The K_{D} for binding to SEQ ID NO: 5 is around 300 PM to around 400 pM.

The first specific binding molecule may bind to an epitope consisting of residues 367 to 379 of SEQ ID NO: 1. This epitope may be bound by the CDRs of the specific binding molecule referred to as “S1G2” herein.

The first specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 54 (SYRADGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS);
VLCDR3 comprises the sequence set forth in SEQ ID NO: 73 (GSSDRTPYTGV);
or for each CDR sequence, an amino acid sequence with
(i) at least 85% identity thereto, and/or
(ii) one, two, or three amino acid substitutions relative thereto.

The first specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 54 (SYRADGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS); and
VLCDR3 comprises the sequence set forth in SEQ ID NO: 73 (GSSDRTPYTGV).

The first specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 447 (QVQLQESGPSLVKPSQTLSLTCTVSGFSLT);
VHFR2 comprises the sequence set forth in SEQ ID NO: 448 (WVRQAPGKAPEWVA);
VHFR3 comprises the sequence set forth in SEQ ID NO: 449 (RLSITRDTSKSQVSLSLSSVTSEDTAVYYCGR);
VHFR4 comprises the sequence set forth in SEQ ID NO: 450 (WGPGLLVTVSS);
VLFR1 comprises the sequence set forth SEQ in ID NO: 451 (QAVVTQPSSVSGSLGQRVSITC);
VLFR2 comprises the sequence set forth in SEQ ID NO: 452 (WFQQLPGSGLRTIIV);
VLFR3 comprises the sequence set forth in SEQ ID NO: 453 (GVPDRFSMSKSGNTATLTISSLQAEDEADYFC);
VLFR4 comprises the sequence set forth in SEQ ID NO: 454 (FGSGTRLTVLG);
or for each FR sequence, an amino acid sequence with
(i) at least 50% identity thereto, and/or
(ii) one, two, three, four or five amino acid substitutions relative thereto.

The first specific binding molecule may comprise:
(a) A VH domain comprising the sequence set forth in SEQ ID NO: 455 (QVQLQESGPSLVKPSQTLSLTCTVSGFSLTSNSVGWVRQAPGKAPEWVAGIDTDGEEG YNPALNSRLSITRDTSKSQVSLSLSSVTSEDTAVYYCGRSYRADGLAYGYVQAIDYWGPG LLVTVSS); and/or
(b) a VL domain comprising the sequence set forth in SEQ ID NO: 456 (QAVVTQPSSVSGSLGQRVSITCSGSFIGISSVGWFQQLPGSGLRTIIVASDGRPSGVPDR FSMSKSGNTATLTISSLQAEDEADYFCGSSDRTPYTGVFGSGTRLTVLG);
or a humanized variant thereof.

The first specific binding molecule may specifically bind to a polypeptide or protein molecule comprising an amino acid sequence comprising residues 367 to 379 of SEQ ID NO: 1 with a K_{D} of less than around 500 pM, optionally wherein the specific binding is measured by surface plasmon resonance (SPR) and optionally wherein
i. The K_{D} for binding to SEQ ID NO: 1 is around 100 pM to around 200 pM, and/or
ii. The K_{D} for binding to SEQ ID NO: 5 is around 400 pM to around 500 pM.

The first specific binding molecule may compete for binding to SEQ ID NO: 1 with any specific binding molecule disclosed herein that binds to an epitope within residues 297 to 391 of SEQ ID NO: 1. The first specific binding molecule may compete with S1D12 or S1G2 for binding to SEQ ID NO: 1.

The person skilled in the art can identify competing antibodies without undertaking undue experimentation or the need to exercise inventive ingenuity (e.g., by using routine competition binding assays). As used herein, a specific binding molecule that competes with another specific binding molecule does so by competition that involves specific binding to the same target.

In a preferred embodiment, the first specific binding molecule may comprise the CDRs, optionally further comprising the framework regions, optionally comprising the VH and/or VL domains, of a specific binding molecule selected from the group consisting of S1D12, S1G2 and CA4.

The first specific binding molecule may bind to an epitope consisting of residues 355 to 367 of SEQ ID NO: 1. Accordingly, the epitope may be within the amino acid sequence of SEQ ID NO: 330 (GSLDNITHVPGGG). This epitope may be bound by the CDRs of the specific binding molecule referred to as “CA4” herein.

The first specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 83 (SYSVY);
VHCDR2 comprises the sequence set forth in SEQ ID NO: 84 (IMYASGRVDYNPALKS);
VHCDR3 comprises the sequence set forth in SEQ ID NO: 89 (GIEN);
VLCDR1 comprises the sequence set forth in SEQ ID NO: 91 (RTSQSVNNYLS);
VLCDR2 comprises the sequence set forth in SEQ ID NO: 95 (YATRLYT); and
VLCDR3 comprises the sequence set forth in SEQ ID NO: 97 (LQYDSTPLA);
or for each CDR sequence, an amino acid sequence with
(i) at least 85% identity thereto, and/or
(ii) one, two, or three amino acid substitutions relative thereto.

The first specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 83 (SYSVY);
VHCDR2 comprises the sequence set forth in SEQ ID NO: 84 (IMYASGRVDYNPALKS);
VHCDR3 comprises the sequence set forth in SEQ ID NO: 89 (GIEN);
VLCDR1 comprises the sequence set forth in SEQ ID NO: 91 (RTSQSVNNYLS);
VLCDR2 comprises the sequence set forth in SEQ ID NO: 95 (YATRLYT); and
VLCDR3 comprises the sequence set forth in SEQ ID NO: 97 (LQYDSTPLA).

The first specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 555 (QVQLQESGPSLVKPSQTLSLTCTVSGFSLT);
VHFR2 comprises the sequence set forth in SEQ ID NO: 556 (WVRQAPGQALEWIS);
VHFR3 comprises the sequence set forth in SEQ ID NO: 557 (RLSITRDTSKSQFSLSLSSVTTEDTAVYYCTR);
VHFR4 comprises the sequence set forth in SEQ ID NO: 558 (WGPGLLVTVSS);
VLFR1 comprises the sequence set forth in SEQ ID NO: 559 (DIQVTQSPSSLSASLTERVSITC);
VLFR2 comprises the sequence set forth in SEQ ID NO: 560 (WYQQKPGQAPKLLIY);
VLFR3 comprises the sequence set forth in SEQ ID NO: 561 (DVPSRFSGSGSGTDYTLTITSLEADDTATYYC);
VLFR4 comprises the sequence set forth in SEQ ID NO: 562 (FGGGTNVEIK);
or for each FR sequence, an amino acid sequence with
   (i) at least 50% identity thereto, and/or
   (ii) one, two, three, four or five amino acid substitutions relative thereto.

The first specific binding molecule may comprise:
(a) A VH domain comprising the sequence set forth in SEQ ID NO: 563 (QVQLQESGPSLVKPSQTLSLTCTVSGFSLTSYSVYWVRQAPGQALEWISIMYASGRVDY NPALKSRLSITRDTSKSQFSLSLSSVTTEDTAVYYCTRGIENWGPGLLVTVSS); and/or
(b) a VL domain comprising the sequence set forth in SEQ ID NO: 564 (DIQVTQSPSSLSASLTERVSITCRTSQSVNNYLSWYQQKPGQAPKLLIYYATRLYTDVPS RFSGSGSGTDYTLTITSLEADDTATYYCLQYDSTPLAFGGGTNVEIK);
or a humanized variant thereof.

The first specific binding molecule may compete with CA4 for binding to SEQ ID NO: 1.

The first specific binding molecule may compete with S1D12, S1G2 or CA4 for binding to SEQ ID NO: 1.

In a preferred embodiment, the first specific binding molecule may comprise the CDRs, optionally further comprising the framework regions, optionally comprising the VH and/or VL domains, of a specific binding molecule selected from the group consisting of S1D12 and S1G2.

The first specific binding molecule may comprise the CDRs of S1D12, S1G2 or CA4, or an alternative specific binding molecule with a nearby or overlapping epitope to any one or more of S1D12, S1G2 or CA4. For instance, the first specific binding molecule may be any specific binding molecule disclosed herein that binds to an epitope within or overlapping residues 307 to 391 of SEQ ID NO: 1. The first specific binding molecule may be any specific binding molecule disclosed herein that binds to an epitope within or overlapping residues 337 to 379 of SEQ ID NO: 1. The first specific binding molecule may comprise the CDR sequences of a clone set out in Table 1, Table 2, Table 3, Table 4, Table 9, Table 10 (wherein the epitope is within or overlapping residues 307 to 391 of SEQ ID NO: 1) or Table 11.

### Second Specific Binding Molecule

The method may further comprise contacting the sample with a second specific binding molecule. The second specific binding molecule binds to an epitope within SEQ ID NO: 1. Contacting the sample with a second specific binding molecule may be a separate step to contacting the sample with the first specific binding molecule. Contacting the sample with the second specific binding molecule is after contacting the sample with the first specific binding molecule. The following describes the second specific binding molecule. Any references under this sub-heading to a or the specific binding molecule (which are not qualified as relating to the first specific binding molecule) are to the first specific binding molecule.

The second specific binding molecule may be any specific binding molecule disclosed herein, such as a specific binding molecule described in PCT application no. PCT/EP2021/069160, wherein the second specific binding molecule is different to the first specific binding molecule. The second specific binding molecule may be a specific binding molecule that binds to an epitope within SEQ ID NO: 1 with a binding affinity greater than the binding affinity with which antibody mAb423 binds to an epitope within SEQ ID NO: 1.

The epitope of the second specific binding molecule may be within SEQ ID NO: 3, SEQ ID NO: 4, SEQ ID NO: 5, SEQ ID NO: 6 or SEQ ID NO: 7.

With the proviso that the second specific binding molecule is different to the first specific binding molecule, the second specific binding molecule may have any combination of features described for the first specific binding molecule. For instance, the epitope and/or any one or more sequences of the second specific binding molecule may be as described above for the first specific binding molecule. Alternatively, the epitope and/or any one or more sequences of the second specific binding molecule may be different to those as described above for the first specific binding molecule. The epitope of the second specific binding molecule may not be within SEQ ID NO: 4, SEQ ID NO: 5, SEQ ID NO: 6 or SEQ ID NO: 7.

The second specific binding molecule may be a known specific binding molecule such as HT7, BT2, Tau12 or Tau146. The second specific binding molecule may be HT7 or BT2. BT2 and HT7 are described in U.S. Pat. No. 6,010,913A and in the article Merken et al 1992-Affinity Purification of Human tau Proteins and the Construction of a Sensitive Sandwich Enzyme-Linked Immunosorbent Assay for Human tau Detection, both of which are hereby incorporated by reference in their entirety.

The second specific binding molecule may bind to an epitope within residues 151 to 243 of SEQ ID NO: 1.

The second specific binding molecule may bind to an epitope consisting of residues 194 to 198 of SEQ ID NO: 1. The second specific binding molecule may be BT2.

The second specific binding molecule may bind to an epitope consisting of residues 159 to 163 of SEQ ID NO: 1. The second specific binding molecule may be HT7.

The second specific binding molecule may compete with BT2 or HT7 for binding to SEQ ID NO: 1.

The second specific binding molecule may comprise the CDRs of HT7, or an alternative specific binding molecule disclosed herein with a nearby or overlapping epitope, such as 3aA6 and 3aD6 which bind to a nearby epitope within residues 147 to 157 of SEQ ID NO: 1.

Alternative second specific binding molecules to HT7 and/or BT2 may comprise the CDRs of the specific binding molecules (optionally further comprising the FW regions and optionally the VH and/or VL domains) of the clones set out in Table 8. For example, the second specific binding molecule may comprise the CDRs of 3aA6 or 3aD6. The second specific binding molecule may comprise the CDRs and FW regions of 3aA6 or 3aD6. The second specific binding molecule may comprise the VH and/or VL domain of 3aA6 or 3aD6.

The second specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 198 (SNAVI);
VHCDR2 comprises the sequence set forth in SEQ ID NO: 200 (LIDVDGDAAYDPALKS);
VHCDR3 comprises the sequence set forth in SEQ ID NO: 202 (DYGSWGYVSDIDY);
VLCDR1 comprises the sequence set forth in SEQ ID NO: 204 (SGSDIGGADVG);
VLCDR2 comprises the sequence set forth in SEQ ID NO: 206 (DNDNRPS); and
VLCDR3 comprises the sequence set forth in SEQ ID NO: 208 (GTYSGANYGI);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 147 to 157 of SEQ ID NO: 1. The specific binding molecule comprising CDRs having 100% identity to those given above is referred to as “3aD6” herein.

The second specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 198 (SNAVI);
VHCDR2 comprises the sequence set forth in SEQ ID NO: 200 (LIDVDGDAAYDPALKS);
VHCDR3 comprises the sequence set forth in SEQ ID NO: 202 (DYGSWGYVSDIDY);
VLCDR1 comprises the sequence set forth in SEQ ID NO: 204 (SGSDIGGADVG);
VLCDR2 comprises the sequence set forth in SEQ ID NO: 206 (DNDNRPS); and
VLCDR3 comprises the sequence set forth in SEQ ID NO: 208 (GTYSGANYGI).

The second specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 17 (SNAVG);
VHCDR2 comprises the sequence set forth in SEQ ID NO: 201 (LIDIDGDTAYNPALES);
VHCDR3 comprises the sequence set forth in SEQ ID NO: 203 (HYDKWGYADSIDY);
VLCDR1 comprises the sequence set forth in SEQ ID NO: 138 (SGSSSNVGYGDYVG);
VLCDR2 comprises the sequence set forth in SEQ ID NO: 207 (DATTRAS); and
VLCDR3 comprises the sequence set forth in SEQ ID NO: 209 (ASYQNERSGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 147 to 157 of SEQ ID NO: 1. The specific binding molecule comprising CDRs having 100% identity to those given above is referred to as “3aA6” herein.

The second specific binding molecule may bind to a polypeptide or protein molecule comprising an amino acid sequence comprising residues 147 to 157 of SEQ ID NO: 1 with a K_{D} of less than around 50 nM. The K_{D} may be less than around 40 nM, less than around 30 nM, or less than around 20 nM. The K_{D} may preferably be for binding to SEQ ID NO: 1 or to SEQ ID NO: 191. The K_{D} for binding to SEQ ID NO: 1 may be around 10 nM to around 20 nM. The K_{D} for binding to SEQ ID NO: 1 may be around 16.5 nM, optionally wherein the specific binding molecule comprises the CDRs of 3aD6.

The second specific binding molecule may comprise an amino acid sequence having at least 70%, at least 75%, at least 80%, at least 90%, at least 95% or at least 99% identity to SEQ ID NO: 418 wherein the specific binding molecule binds to a polypeptide or protein molecule comprising an amino acid sequence comprising residues 147 to 157 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 418. The CDRs may be 100% identical to the CDRs of SEQ ID NO: 418. The specific binding molecule may comprise an amino acid sequence having at least 70%, at least 75%, at least 80%, at least 90%, at least 95% or at least 99% identity to SEQ ID NO: 418 wherein CDRs are 100% identical to the CDRs of SEQ ID NO: 418. The specific binding molecule may comprise the amino acid sequence of SEQ ID NO: 418.

(3aA6 amino acid sequence)
SEQ ID NO: 418
QVRLQESGSSLVKPSQTLSLVCTVSGFPLTSNAVGWVRQAPGKAPEWLG
LIDIDGDTAYNPALESRLSITRDTSKSQVSLSLSSVAIEDTAVYYCARH
YDKWGYADSIDYWGPGLLVTVSSEGKSSGASGESKVDDQALLTQPSSVF
GSLGQRVSITCSGSSSNVGYGDYVGWYQQVPGSAPKLLIYDATTRASGV
PDRFSGSRSGNTATLTISSLQAEDEADYYCASYQNERSGVFGSGTRLTV
LG

The second specific binding molecule may comprise an amino acid sequence having at least 70%, at least 75%, at least 80%, at least 90%, at least 95% or at least 99% identity to SEQ ID NO: 419 wherein the specific binding molecule binds to a polypeptide or protein molecule comprising an amino acid sequence comprising residues 147 to 157 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 419. The CDRs may be 100% identical to the CDRs of SEQ ID NO: 419. The specific binding molecule may comprise an amino acid sequence having at least 70%, at least 75%, at least 80%, at least 90%, at least 95% or at least 99% identity to SEQ ID NO: 419 wherein CDRs are 100% identical to the CDRs of SEQ ID NO: 419. The specific binding molecule may comprise the amino acid sequence of SEQ ID NO: 419.

(3aD6 amino acid sequence)
SEQ ID NO: 419
QVQLQESGPSLVKPSQTLSLTCTVSGFSLTSNAVIWVRQAPGKAPEWVA
LIDVDGDAAYDPALKSRLSITRDTSKSQVSLSLRSVTTEDTAVYYCARD
YGSWGYVSDIDYWGPGLLVTVSSEGKSSGASGESKVDDQAVLTQPSSVS
GSLGQRVSITCSGSDIGGADVGWFQQVPGSGLRTLIYDNDNRPSGVPDR
FSGSKSGNTATLTISSLQPEDEADYFCGTYSGANYGIFGSGTRLTVLG

The second specific binding molecule may comprise the CDRs of BT2, or an alternative specific binding molecule disclosed herein with a nearby or overlapping epitope.

Alternative second specific binding molecules to HT7 and/or BT2 may comprise the CDRs of the specific binding molecules (optionally further comprising the FW regions and optionally the VH and/or VL domains) of the clones set out in Table 10. For example, the second specific binding molecule may comprise the CDRs of 3bD11, CB11, CA2, CB6, CA7, CA8, CB10, CC7, CB12, CC3, CA1, CA3, CD2, CC4, CD1 or CC5. The second specific binding molecule may comprise the CDRs and FW regions of 3bD11, CB11, CA2, CB6, CA7, CA8, CB10, CC7, CB12, CC3, CA1, CA3, CD2, CC4, CD1 or CC5. The second specific binding molecule may comprise the VH and/or VL domain of 3bD11, CB11, CA2, CB6, CA7, CA8, CB10, CC7, CB12, CC3, CA1, CA3, CD2, CC4, CD1 or CC5.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises a VHCDR1 amino acid sequence set forth in table 10;
VHCDR2 comprises a VHCDR2 amino acid sequence set forth in table 10;
VHCDR3 comprises a VHCDR3 amino acid sequence set forth in table 10;
VLCDR1 comprises a VLCDR1 amino acid sequence set forth in table 10;
VLCDR2 comprises a VLCDR2 amino acid sequence set forth in table 10; and
VLCDR3 comprises a VLCDR3 amino acid sequence set forth in table 10;
or for each CDR sequence, an amino acid sequence with
   (i) at least 85% identity thereto, and/or
   (ii) one, two, or three amino acid substitutions relative thereto,
wherein the specific binding molecule binds to an epitope within SEQ ID NO: 1. VHCDR1, VHCDR2, VHCDR3, VLCDR1, VLCDR2 and VLCDR3 may be from a single clone set out in table 10. The clone may be selected from the group consisting of 3bD11, CB11, CA2, CB6, CA7, CA8, CB10, CC7, CB12, CC3, CA1, CA3, CD2, CC4, CD1 and CC5.
   General Features of First and/or Second Specific Binding Molecules

The following describes the features of specific binding molecules in general and includes details of the specific binding molecules described in PCT application no. PCT/EP2021/069160. Any references under this sub-heading to a or the specific binding molecule (which are not qualified as relating specifically to either the first specific binding molecule or the second specific binding molecule) are to specific binding molecules in general and are explicitly contemplated in combination with any features of the first specific binding molecule or the second specific binding molecules, with the proviso that the first specific binding molecule binds to an epitope within residues 297 to 391 of SEQ ID NO: 1 (therefore references to specific binding molecules disclosed herein that do not bind to an epitope within residues 297 to 391 of SEQ ID NO: 1 refer to the second specific binding molecule only).

The epitope of the specific binding molecule may be within residues 297 to 391 of SEQ ID NO: 1. Residues 297 to 391 of full-length Tau are also known as the predominant fragment isolated from proteolytically stable core of the paired helical filament (PHF) or the PHF-core fragment. Therefore, the epitope of the specific binding molecule may be within the PHF or within the dGAE fragment. Accordingly, the epitope of the specific binding molecule may be within SEQ ID NO 4.

The epitope of the specific binding molecule may be within residues 297 to 390 of SEQ ID NO: 1. Residues 297 to 390 of full-length Tau are also known as the dGA fragment. Therefore, the epitope of the specific binding molecule may be within the dGA fragment. Accordingly, the epitope of the specific binding molecule may be within SEQ ID NO: 5. The epitope of the specific binding molecule may be within dGAE73 and/or dGAE71. Accordingly, the epitope of the specific binding molecule may be within SEQ ID NO: 6 and/or SEQ ID NO: 7.

The epitope of the specific binding molecule may be within residues 308 to 378 of SEQ ID NO: 1. Residues 308 to 378 of full-length Tau are also known as the PHF core. Therefore, the epitope of the specific binding molecule may be within the PHF core. Accordingly, the epitope of the specific binding molecule may be within SEQ ID NO: 7.

The epitope of the specific binding molecule may be within residues 297 to 386 of SEQ ID NO: 1. The epitope of the specific binding molecule may be within residues 306 to 391 of SEQ ID NO: 1. The epitope of the specific binding molecule may be within residues 306 to 386 of SEQ ID NO: 1.

The epitope of the specific binding molecule may be within an amino acid sequence selected from the group consisting of residues 306 to 391 of SEQ ID NO: 1, residues 307 to 391 of SEQ ID NO: 1, residues 337 to 355 of SEQ ID NO: 1, residues 367 to 379 of SEQ ID NO: 1, residues 331 to 360 of SEQ ID NO: 1, residues 355 to 367 of SEQ ID NO: 1, residues 379 to 391 of SEQ ID NO: 1, residues 297 to 390 of SEQ ID NO: 1, residues 369 to 390 of SEQ ID NO: 1, residues 337 to 368 of SEQ ID NO: 1, residues 337 to 379 of SEQ ID NO: 1, residues 412 to 441 of SEQ ID NO: 1, residues 1 to 49 of SEQ ID NO: 1, residues 49 to 111 of SEQ ID NO: 1, residues 147 to 157 of SEQ ID NO: 1, residues 1 to 155 of SEQ ID NO: 1, residues 1 to 238 of SEQ ID NO: 1, residues 1 to 319 of SEQ ID NO: 1, residues 13 to 25 of SEQ ID NO: 1, residues 49 to 113 of SEQ ID NO: 1, residues 49 to 155 of SEQ ID NO: 1, residues 49 to 238 of SEQ ID NO: 1, residues 113 to 238 of SEQ ID NO: 1, residues 155 to 227 of SEQ ID NO: 1, residues 155 to 238 of SEQ ID NO: 1, residues 186 to 263 of SEQ ID NO: 1, residues 186 to 350 of SEQ ID NO: 1, residues 239 to 348 of SEQ ID NO: 1, residues 266 to 359 of SEQ ID NO: 1, residues 277 to 319 of SEQ ID NO: 1, residues 319 to 331 of SEQ ID NO: 1, residues 348 to 390 of SEQ ID NO: 1, residues 348 to 441 of SEQ ID NO: 1, residues 359 to 391 of SEQ ID NO: 1, and residues 360 to 390 of SEQ ID NO: 1.

The epitope of the specific binding molecule may be within an amino acid sequence selected from the group consisting of residues 337 to 355 of SEQ ID NO: 1, residues 367 to 379 of SEQ ID NO: 1, residues 331 to 360 of SEQ ID NO: 1, residues 355 to 367 of SEQ ID NO: 1, residues 379 to 391 of SEQ ID NO: 1, residues 297 to 390 of SEQ ID NO: 1, residues 369 to 390 of SEQ ID NO: 1, residues 337 to 368 of SEQ ID NO: 1, residues 412 to 441 of SEQ ID NO: 1, residues 1 to 49 of SEQ ID NO: 1, residues 49 to 111 of SEQ ID NO: 1, and residues 147 to 157 of SEQ ID NO: 1.

The epitope of the specific binding molecule may be within an amino acid sequence selected from the group consisting of residues 337 to 355 of SEQ ID NO: 1, residues 367 to 379 of SEQ ID NO: 1, residues 331 to 360 of SEQ ID NO: 1 and residues 355 to 367 of SEQ ID NO: 1.

The epitope of the specific binding molecule may be within an amino acid sequence selected from the group consisting of residues 341 to 353 of SEQ ID NO: 1.

The epitope of specific binding molecules of the invention may be any amino acid sequence of SEQ ID NO: 1 indicated as containing critical binding residues by ELISA or alanine scanning mutagenesis, as described for example in Examples 5 to 12.

Epitopes described herein may be identified as “comprising” a certain amino acid sequence or by the phrase “the specific binding molecule binds to a polypeptide or protein molecule comprising an amino acid sequence comprising residues . . . ”. As will be apparent to the skilled person, when a specific binding molecule binds a polypeptide or protein molecule comprising its epitope, it will also bind a polypeptide or protein molecule consisting of its epitope. As used herein, the phrase “the specific binding molecule binds to a polypeptide or protein molecule comprising an amino acid sequence comprising residues . . . ” may therefore alternatively be substituted wherever it occurs for the phrase “the specific binding molecule binds to a polypeptide or protein molecule comprising an amino acid sequence consisting of residues . . . ”; the phrase “the specific binding molecule binds to a polypeptide or protein molecule consisting of an amino acid sequence comprising residues . . . ”; or the phrase “the specific binding molecule binds to a polypeptide or protein molecule consisting of an amino acid sequence consisting of residues . . . ”.

The skilled person is aware that not all residues within an epitope are always essential. A specific binding molecule may retain binding to an amino acid sequence with at least 70% identity to an epitope. The specific binding molecule may bind to any of the epitopes disclosed herein or an amino acid sequence having at least 70% identity thereto.

A specific binding molecule may retain binding to an amino acid sequence with at least 70%, at least 75%, at least 80%, at least 90%, at least 95% or at least 99% identity to an epitope. The specific binding molecule may bind to any of the epitopes disclosed herein or an amino acid sequence having at least 70%, at least 75%, at least 80%, at least 90%, at least 95% or at least 99% identity thereto.

In embodiments of the invention where specific binding molecule retains binding to an amino acid sequence with less than 100% sequence identity to the amino acid sequence of SEQ ID NO: 1 (or any of SEQ ID NOs: 3 to 7 or any other epitope defined herein), the epitope sequence may be altered by substitution, addition or deletion of an appropriate number of amino acids in the sequences of SEQ ID NO: 1 (or any of SEQ ID NOs: 3 to 7 or any other epitope defined herein). In another embodiment of the invention, the epitope may be modified by the substitution, addition or deletion of up to 2 amino acids relative to SEQ ID NO: 1 (or any of SEQ ID NOs: 3 to 7 or any other epitope defined herein), with the proviso that the resultant epitope sequence has at least 85% or 90% sequence identity to SEQ ID NO: 1 (or any of SEQ ID NOs: 3 to 7 or any other epitope defined herein), as set out above. By “substitution, addition or deletion” is included combinations of substitutions, additions and deletions.

When an epitope sequence is modified by substitution of a particular amino acid residue, the substitution may be a conservative amino acid substitution. The term “conservative amino acid substitution”, as used herein, refers to an amino acid substitution in which one amino acid residue is replaced with another amino acid residue having a similar side chain. Amino acids with similar side chains tend to have similar properties, and thus a conservative substitution of an amino acid important for the structure or function of a polypeptide may be expected to affect polypeptide structure/function less than a non-conservative amino acid substitution at the same position. Families of amino acid residues having similar side chains have been defined in the art, including basic side chains (e.g. lysine, arginine, histidine), acidic side chains (e.g. aspartic acid, glutamic acid), uncharged polar side chains (e.g. asparagine, glutamine, serine, threonine, tyrosine), non-polar side chains (e.g. glycine, cysteine, alanine, valine, leucine, isoleucine, proline, phenylalanine, methionine, tryptophan) and aromatic side chains (e.g. tyrosine, phenylalanine, tryptophan, histidine). Thus a conservative amino acid substitution may be considered to be a substitution in which a particular amino acid residue is substituted for a different amino acid in the same family. However, a substitution of an epitope residue may equally be a non-conservative substitution, in which one amino acid is substituted for another with a side-chain belonging to a different family.

The epitope may be at least five, at least six, at least seven, at least eight, at least nine, at least 10, at least 11, at least 12, at least 13, at least 14, at least 15, at least 16, at least 17, at least 18, at least 19 or at least 20 amino acids in length. The epitope may be five to 20 amino acids in length. The epitope may be five to 15 amino acids in length. The epitope may be five to 12 amino acids in length. The epitope may be six to 12 amino acids in length. The epitope may be seven to 12 amino acids in length.

As used herein, the term “within” means “contained within” or “fully within”. No residues thought to be essential for the binding of the specific binding molecule to its target are outside of the epitope. Residues outside the epitope do not significantly contribute to binding. For example, where the epitope of the specific binding molecule is within residues 337 to 355 of SEQ ID NO: 1, residues outside of residues 337 to 355 do not significantly contribute to binding.

The epitope may comprise any residues within SEQ ID NO: 1 bound by the specific binding molecule. The epitope may be a continuous epitope or a discontinuous epitope.

A continuous epitope may be any consecutive residues within SEQ ID NO: 1 bound by the specific binding molecule. Consecutive residues are adjacent to one another in the primary structure of a polypeptide.

A discontinuous epitope may be any non-consecutive residues within SEQ ID NO: 1 bound by the specific binding molecule. Discontinuous epitopes are typically formed by non-consecutive residues adopting nearby positions in three-dimensional space due to the folding of a polypeptide.

Typically, a specific binding molecule binds to a polypeptide or protein molecule comprising its epitope. Therefore, the specific binding molecule may bind to SEQ ID NO: 1 or a fragment thereof. The specific molecule may bind to SEQ ID NO: 3, SEQ ID NO: 4, SEQ ID NO: 5, SEQ ID NO: 6 and/or SEQ ID NO: 7. The specific molecule may bind to the PHF or the dGAE fragment. the specific binding molecule may bind to the dGA fragment. The specific binding molecule may bind to the PHF core. The specific binding molecule may bind to a polypeptide or protein molecule comprising an amino acid sequence selected from the group consisting of residues 337 to 355 of SEQ ID NO: 1, residues 367 to 379 of SEQ ID NO: 1, residues 331 to 360 of SEQ ID NO: 1, residues 355 to 367 of SEQ ID NO: 1, residues 379 to 391 of SEQ ID NO: 1, residues 297 to 390 of SEQ ID NO: 1, residues 369 to 390 of SEQ ID NO: 1, residues 337 to 368 of SEQ ID NO: 1, residues 412 to 441 of SEQ ID NO: 1, residues 1 to 49 of SEQ ID NO: 1, residues 49 to 111 of SEQ ID NO: 1, residues 147 to 157 of SEQ ID NO: 1, residues 1 to 155 of SEQ ID NO: 1, residues 1 to 238 of SEQ ID NO: 1, residues 1 to 319 of SEQ ID NO: 1, residues 13 to 25 of SEQ ID NO: 1, residues 49 to 113 of SEQ ID NO: 1, residues 49 to 155 of SEQ ID NO: 1, residues 49 to 238 of SEQ ID NO: 1, residues 113 to 238 of SEQ ID NO: 1, residues 155 to 227 of SEQ ID NO: 1, residues 155 to 238 of SEQ ID NO: 1, residues 186 to 263 of SEQ ID NO: 1, residues 186 to 350 of SEQ ID NO: 1, residues 239 to 348 of SEQ ID NO: 1, residues 266 to 359 of SEQ ID NO: 1, residues 277 to 319 of SEQ ID NO: 1, residues 319 to 331 of SEQ ID NO: 1, residues 348 to 390 of SEQ ID NO: 1, residues 348 to 441 of SEQ ID NO: 1, residues 359 to 391 of SEQ ID NO: 1, and residues 360 to 390 of SEQ ID NO: 1.

The specific binding molecule may bind to a polypeptide or protein molecule comprising an amino acid sequence of residues 341 to 353 of SEQ ID NO: 1.

In the sequences herein, “/” means “or” and denotes residues that the inventors have shown may vary as specified. In this context, “-” means a gap or no amino acid. X is any amino acid. For example, “N/S” means a residue which may be either N or S. Likewise, “G/-” means a residue which may be either G or absent. Likewise, “H/F/Y” means a residue may be H, F or Y. Where sequence identity values are specified, sequence identity may be calculated starting from any one of the residues separated by a “/”.

The epitope of the specific binding molecule may be within an amino acid sequence comprising residues 337 to 355 of SEQ ID NO: 1. The epitope of the specific binding molecule within an amino acid sequence comprising residues 337 to 355 of SEQ ID NO: 1 may be within an amino acid sequence comprising residues 341 to 353 of SEQ ID NO: 1. Accordingly, the epitope may be within the amino acid sequence of SEQ ID NO: 8 (VEVKSEKLDFKDR).

The epitope may be within an amino acid sequence comprising residues 337 to 349 of SEQ ID NO: 1, preferably within an amino acid sequence comprising residues 337 to 355 of SEQ ID NO: 1. This epitope may be bound by the CDRs of the specific binding molecule referred to as “S1D12” herein. The epitope may comprise the amino acid sequence of SEQ ID NO: 8 (VEVKSEKLDFKDR). The epitope may comprise an amino acid sequence having at least 70%, at least 75%, at least 80%, at least 90%, at least 95% or at least 99% identity to SEQ ID NO: 8 (VEVKSEKLDFKDR). Critical residues of the epitope may be residues 343 (K), 346 (F) and/or 349 (R) (numbering according to SEQ ID NO: 1). The epitope may comprise the amino acid sequence of SEQ ID NO: 9 (XXXXXXXKXXFXXR, wherein X is any amino acid). The epitope may comprise the amino acid sequence of SEQ ID NO: 8, wherein any one or more residue other than residue number 343 (K), 346 (F) and/or 349 (R) is replaced by a non-conservative amino acid substitution (numbering according to SEQ ID NO: 1). The epitope may comprise the amino acid sequence of SEQ ID NO: 8, wherein any one or more residue other than residue number 343 (K), 346 (F) and/or 349 (R) is replaced by a conservative amino acid substitution (numbering according to SEQ ID NO: 1). The epitope may comprise the amino acid sequence of SEQ ID NO: 8, wherein any one or more residue other than residue number 343 (K), 346 (F) and/or 349 (R) is replaced by a conservative amino acid substitution (numbering according to SEQ ID NO: 1) and any one or more residue other than residue number 343 (K), 346 (F) and/or 349 (R) is replaced by a non-conservative amino acid substitution (numbering according to SEQ ID NO: 1).

The epitope may consist of residues 337 to 349 of SEQ ID NO: 1, preferably residues 337 to 355 of SEQ ID NO: 1. The epitope may consist of the amino acid sequence of SEQ ID NO: 8 (VEVKSEKLDFKDR). The epitope may consist of an amino acid sequence having at least 70%, at least 75%, at least 80%, at least 90%, at least 95% or at least 99% identity to SEQ ID NO: 8 (VEVKSEKLDFKDR).

The specific binding molecule may bind to a polypeptide or protein molecule comprising an amino acid sequence comprising residues 337 to 355 of SEQ ID NO: 1. Said specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 10 (N/S N A V G);
VHCDR2 comprises the sequence set forth in SEQ ID NO: 11 (G C S S D G T/K C Y Y/H N S A L K S);
VHCDR3 comprises the sequence set forth in SEQ ID NO: 12 (G H/F/Y Y S/P I/V Y G Y D Y L/S G T I D Y);
VLCDR1 comprises the sequence set forth in SEQ ID NO: 13 (S G S S S N V G/-G G/R N S/D V G/A);
VLCDR2 comprises the sequence set forth in SEQ ID NO: 14 (D/N/G T N/T S R P S);
VLCDR3 comprises the sequence set forth in SEQ ID NO: 15 (VIA T/S G D S T/S T/A H/I D/N D L/I);
or for each CDR sequence, an amino acid sequence with
   (i) at least 85% identity thereto, and/or
   (ii) one, two, or three amino acid substitutions relative thereto.

Said sequence identity is at least about 85% sequence identity and may therefore be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity. Preferably said sequence identity is at least 90% or at least 95%.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 16 (NNAVG) or SEQ ID NO: 17 (SNAVG);
VHCDR2 comprises the sequence set forth in SEQ ID NO: 18 (GCSSDGTCYYNSALKS), SEQ ID NO: 19 (GCSSDGKCYHNSALKS) or SEQ ID NO: 20 (GCSSDGKCYYNSALKS);
VHCDR3 comprises the sequence set forth in SEQ ID NO: 21 (GHYSIYGYDYLGTIDY), SEQ ID NO: 22 (GFYSIYGYDYSGTIDY), or SEQ ID NO: 23 (GYYPVYGYDYLGTIDY);
VLCDR1 comprises the sequence set forth in SEQ ID NO: 24 (SGSSSNVGGGNSVG) or SEQ ID NO: 25 (SGSSSNVGRNDVA);
VLCDR2 comprises the sequence set forth in SEQ ID NO: 26 (DTNSRPS), SEQ ID NO: 27 (NTNSRPS), or SEQ ID NO: 28 (GTTSRPS);
VLCDR3 comprises the sequence set forth in SEQ ID NO: 29 (VTGDSTTHDDL), SEQ ID NO: 30 (VTGDSSTHDDL), or SEQ ID NO: 31 (ASGDSSAINDI);
or for each CDR sequence, an amino acid sequence with
   (i) at least 85% identity thereto, and/or
   (ii) one, two, or three amino acid substitutions relative thereto,
   wherein the specific binding molecule binds to a polypeptide or protein molecule comprising an amino acid sequence comprising residues 337 to 355 of SEQ ID NO: 1.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 16 (NNAVG);
VHCDR2 comprises the sequence set forth in SEQ ID NO: 18 (GCSSDGTCYYNSALKS);
VHCDR3 comprises the sequence set forth in SEQ ID NO: 21 (GHYSIYGYDYLGTIDY);
VLCDR1 comprises the sequence set forth in SEQ ID NO: 24 (SGSSSNVGGGNSVG);
VLCDR2 comprises the sequence set forth in SEQ ID NO: 26 (DTNSRPS);
VLCDR3 comprises the sequence set forth in SEQ ID NO: 29 (VTGDSTTHDDL);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 337 to 355 of SEQ ID NO: 1. The specific binding molecule comprising CDRs having 100% identity to those given above is referred to as “S1D12” (or abbreviated to “1D12”) herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 16 (NNAVG);
VHCDR2 comprises the sequence set forth in SEQ ID NO: 18 (GCSSDGTCYYNSALKS);
VHCDR3 comprises the sequence set forth in SEQ ID NO: 21 (GHYSIYGYDYLGTIDY);
VLCDR1 comprises the sequence set forth in SEQ ID NO: 24 (SGSSSNVGGGNSVG);
VLCDR2 comprises the sequence set forth in SEQ ID NO: 26 (DTNSRPS); and
VLCDR3 comprises the sequence set forth in SEQ ID NO: 29 (VTGDSTTHDDL).

Any specific binding molecule disclosed herein may be further defined by reference to one or more framework region (FR). The framework regions (FRs) are non-CDR sequences which together with the CDR sequences form a variable domain.

The VH domain may have the formula: VHFR1-VHCDR1-VHFR2-VHCDR2-VHFR3-VHCDR3-VHFR4.

The VL domain may have the formula: VLFR1-VLCDR1-VLFR2-VLCDR2-VLFR3-VLCDR3-VLFR4.

The skilled person is able to identify CDR and framework regions within the amino acid sequence of a variable domain using known methods described elsewhere herein. Accordingly, any specific binding molecule disclosed herein may be defined by reference to its CDRs and FRs. In some instances, some of the FR residues may contribute to the affinity with which a specific binding molecule binds its target. However, without being bound by theory, function is more likely to be preserved when replacing an FR residue than when replacing a CDR residue. FR residues may for example be commonly replaced by corresponding residues from human sequences during the process of humanization. FR sequences may therefore be more tolerant of amino acid substitutions than CDR sequences.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence from any specific binding molecule disclosed herein; or for each FR sequence, an amino acid sequence with
   (i) at least 50% identity thereto, and/or
   (ii) one, two, three, four or five amino acid substitutions relative thereto; and
(b) CDRs VHCDR1, VHCDR2, VHCDR3, VLCDR1, VLCDR2 and VLCDR3, wherein each of said CDRs comprises an amino acid sequence from any specific binding molecule disclosed herein, or for each CDR sequence, an amino acid sequence with
   (i) at least 85% identity thereto, and/or
   (ii) one, two, or three amino acid substitutions relative thereto.

Said sequence identity in a CDR sequence is at least about 85% sequence identity and may therefore be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity. Preferably said sequence identity is at least 90% or at least 95%.

Said sequence identity in a FR sequence is at least about 50% sequence identity and may therefore be at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity. Preferably said sequence identity is at least 90% or at least 95%.

Where an FR sequence has at least 50% identity (but less than 100% identity) to an FR sequence disclosed as part of a specific binding molecule disclosed herein, the FR sequence may be a humanized sequence. In other words, the changes to amino acid sequence may be only those needed to humanize the sequence.

Where an FR sequence has one, two, three, four or five amino acid substitutions relative to an FR sequence disclosed as part of a specific binding molecule disclosed herein, the FR sequence may be a humanized sequence. In other words, the substitutions may be only those needed to humanize the sequence.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence from any specific binding molecule disclosed herein; and
(b) CDRs VHCDR1, VHCDR2, VHCDR3, VLCDR1, VLCDR2 and VLCDR3, wherein each of said CDRs comprises an amino acid sequence from any specific binding molecule disclosed herein.

Typically, each of the FRs will be from the same specific binding molecule disclosed herein. Typically, each of the CDRs will be from the same specific binding molecule disclosed herein. Typically, there will be no additional amino acid residues intervening between a defined FR and CDR; each of said FRs and each of said CDRs may therefore be said to consist of an amino acid sequence from any specific binding molecule disclosed herein.

As used herein, the phrase “comprising the CDRs” also encompasses a specific binding molecule comprising the CDRs and FRs of a specific binding molecule disclosed herein, including variants of the FRs including those described above, such as humanized FRs. It also encompasses a specific binding molecule comprising the VH and/or VL domains of a specific binding molecule disclosed herein, including variants of the FRs including those described above, such as humanized FRs. It also encompasses a specific binding molecule comprising the heavy chain and/or light chain of a specific binding molecule disclosed herein, including variants of the FRs and constant regions including those described above, such as humanized FRs and humanized constant regions.

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 435 (QVQLQESGPSLVKPSQTLSLTCTVSGFSLN);
VHFR2 comprises the sequence set forth in SEQ ID NO: 436 (WVRQAPGKVPESLV);
VHFR3 comprises the sequence set forth in SEQ ID NO: 437 (RLDITRDTSKNQISLSLSSVTTDDAAVYYCTR);
VHFR4 comprises the sequence set forth in SEQ ID NO: 438 (WGPGLLVTVSS);
VLFR1 comprises the sequence set forth in SEQ ID NO: 439 (QAVLTQPSSVSGSLGQRVSITC);
VLFR2 comprises the sequence set forth in SEQ ID NO: 440 (WYQHLPGSGLKTIIY);
VLFR3 comprises the sequence set forth in SEQ ID NO: 44 (GVPDRFSGSRSGNTATLTINSLQAEDEGDYYC);
VLFR4 comprises the sequence set forth in SEQ ID NO: 442 (VGSGTRLTVLG);
or for each FR sequence, an amino acid sequence with
   (i) at least 50% identity thereto, and/or
   (ii) one, two, three, four or five amino acid substitutions relative thereto.

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 435 (QVQLQESGPSLVKPSQTLSLTCTVSGFSLN);
VHFR2 comprises the sequence set forth in SEQ ID NO: 436 (WVRQAPGKVPESLV);
VHFR3 comprises the sequence set forth in SEQ ID NO: 437 (RLDITRDTSKNQISLSLSSVTTDDAAVYYCTR);
VHFR4 comprises the sequence set forth in SEQ ID NO: 438 (WGPGLLVTVSS);
VLFR1 comprises the sequence set forth in SEQ ID NO: 439 (QAVLTQPSSVSGSLGQRVSITC);
VLFR2 comprises the sequence set forth in SEQ ID NO: 440 (WYQHLPGSGLKTIIY);
VLFR3 comprises the sequence set forth in SEQ ID NO: 441 (GVPDRFSGSRSGNTATLTINSLQAEDEGDYYC);
VLFR4 comprises the sequence set forth in SEQ ID NO: 442 (VGSGTRLTVLG);
or for each FR sequence, an amino acid sequence with
   (i) at least 50% identity thereto, and/or
   (ii) one, two, three, four or five amino acid substitutions relative thereto,
wherein the specific binding molecule binds to a polypeptide or protein molecule comprising an amino acid sequence comprising residues 337 to 355 of SEQ ID NO: 1. A specific binding molecule comprising FRs having 100% identity to those given above is referred to as “S1D12” (or abbreviated to “1D12”) herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 435 (QVQLQESGPSLVKPSQTLSLTCTVSGFSLN);
   VHFR2 comprises the sequence set forth in SEQ ID NO: 436 (WVRQAPGKVPESLV);
   VHFR3 comprises the sequence set forth in SEQ ID NO: 437 (RLDITRDTSKNQISLSLSSVTTDDAAVYYCTR);
   VHFR4 comprises the sequence set forth in SEQ ID NO: 438 (WGPGLLVTVSS);
   VLFR1 comprises the sequence set forth in SEQ ID NO: 439 (QAVLTQPSSVSGSLGQRVSITC);
   VLFR2 comprises the sequence set forth in SEQ ID NO: 440 (WYQHLPGSGLKTIIY);
   VLFR3 comprises the sequence set forth in SEQ ID NO: 441 (GVPDRFSGSRSGNTATLTINSLQAEDEGDYYC);
   VLFR4 comprises the sequence set forth in SEQ ID NO: 442 (VGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 16 (NNAVG);
   VHCDR2 comprises the sequence set forth in SEQ ID NO: 18 (GCSSDGTCYYNSALKS);
   VHCDR3 comprises the sequence set forth in SEQ ID NO: 21 (GHYSIYGYDYLGTIDY);
   VLCDR1 comprises the sequence set forth in SEQ ID NO: 24 (SGSSSNVGGGNSVG);
   VLCDR2 comprises the sequence set forth in SEQ ID NO: 26 (DTNSRPS);
   VLCDR3 comprises the sequence set forth in SEQ ID NO: 29 (VTGDSTTHDDL);
   or for each CDR sequence, an amino acid sequence with
      (i) at least 85% identity thereto, and/or
      (ii) one, two, or three amino acid substitutions relative thereto
   wherein the specific binding molecule binds to a polypeptide or protein molecule comprising an amino acid sequence comprising residues 337 to 355 of SEQ ID NO: 1. The specific binding molecule comprising FRs and CDRs having 100% identity to those given above is referred to as “S1D12” (or abbreviated to “1D12”) herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 435 (QVQLQESGPSLVKPSQTLSLTCTVSGFSLN);
   VHFR2 comprises the sequence set forth in SEQ ID NO: 436 (WVRQAPGKVPESLV);
   VHFR3 comprises the sequence set forth in SEQ ID NO: 437 (RLDITRDTSKNQISLSLSSVTTDDAAVYYCTR);
   VHFR4 comprises the sequence set forth in SEQ ID NO: 438 (WGPGLLVTVSS);
   VLFR1 comprises the sequence set forth in SEQ ID NO: 439 (QAVLTQPSSVSGSLGQRVSITC);
   VLFR2 comprises the sequence set forth in SEQ ID NO: 440 (WYQHLPGSGLKTIIY);
   VLFR3 comprises the sequence set forth in SEQ ID NO: 441 (GVPDRFSGSRSGNTATLTINSLQAEDEGDYYC);
   VLFR4 comprises the sequence set forth in SEQ ID NO: 442 (VGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 16 (NNAVG);
   VHCDR2 comprises the sequence set forth in SEQ ID NO: 18 (GCSSDGTCYYNSALKS);
   VHCDR3 comprises the sequence set forth in SEQ ID NO: 21 (GHYSIYGYDYLGTIDY);
   VLCDR1 comprises the sequence set forth in SEQ ID NO: 24 (SGSSSNVGGGNSVG);
   VLCDR2 comprises the sequence set forth in SEQ ID NO: 26 (DTNSRPS);
   VLCDR3 comprises the sequence set forth in SEQ ID NO: 29 (VTGDSTTHDDL);
   wherein the specific binding molecule binds to a polypeptide or protein molecule comprising an amino acid sequence comprising residues 337 to 355 of SEQ ID NO: 1. The specific binding molecule comprising FRs and CDRs having 100% identity to those given above is referred to as “S1D12” (or abbreviated to “1D12”) herein.

The specific binding molecule may comprise:
(a) A VH domain comprising the sequence set forth in SEQ ID NO: 443 (QVQLQESGPSLVKPSQTLSLTCTVSGFSLNNNAVGWVRQAPGKVPESLVGCSSDGTCY YNSALKSRLDITRDTSKNQISLSLSSVTTDDAAVYYCTRGHYSIYGYDYLGTIDYWGPGLL VTVSS); and/or
(b) a VL domain comprising the sequence set forth in SEQ ID NO: 444 (QAVLTQPSSVSGSLGQRVSITCSGSSSNVGGGNSVGWYQHLPGSGLKTIIYDTNSRPSG VPDRFSGSRSGNTATLTINSLQAEDEGDYYCVTGDSTTHDDLVGSGTRLTVLG);
or a humanized variant thereof.

The specific binding molecule may comprise:
(a) A heavy chain comprising the sequence set forth in SEQ ID NO: 445 (QVQLQESGPSLVKPSQTLSLTCTVSGFSLNNNAVGWVRQAPGKVPESLVGCSSDGTCY YNSALKSRLDITRDTSKNQISLSLSSVTTDDAAVYYCTRGHYSIYGYDYLGTIDYWGPGLL VTVSSAKTTAPSVYPLAPVCGDTTGSSVTLGCLVKGYFPEPVTLTWNSGSLSSGVHTFPA VLQSDLYTLSSSVTVTSSTWPSQSITCNVAHPASSTKVDKKIEPRGPTIKPCPPCKCPAPN LLGGPSVFIFPPKIKDVLMISLSPIVTCVVVDVSEDDPDVQISWFVNNVEVHTAQTQTHRED YNSTLRVVSALPIQHQDWMSGKEFKCKVNNKDLPAPIERTISKPKGSVRAPQVYVLPPPE EEMTKKQVTLTCMVTDFMPEDIYVEWTNNGKTELNYKNTEPVLDSDGSYFMYSKLRVEK KNWVERNSYSCSVVHEGLHNHHTTKSFSRTPGK); and/or
(b) a light chain comprising the sequence set forth in SEQ ID NO: 446 (QAVLTQPSSVSGSLGQRVSITCSGSSSNVGGGNSVGWYQHLPGSGLKTIIYDTNSRPSG VPDRFSGSRSGNTATLTINSLQAEDEGDYYCVTGDSTTHDDLVGSGTRLTVLGGQPKSS PSVTLFPPSSEELETNKATLVCTITDFYPGVVTVDWKVDGTPVTQGMETTQPSKQSNNKY MASSYLTLTARAWERHSSYSCQVTHEGHTVEKSLSRADCS);
or a humanized variant thereof.

The specific binding molecule may comprise the CDR sequences of a clone set out in Table 1 below. The epitope may be within residues 337 to 355 of SEQ ID NO:1.

TABLE 1

| Clone | VH | | | VL | | | |
|---|---|---|---|---|---|---|---|
| name | CDR1 | CDR2 | CDR3 | CDR1 | CDR2 | CDR3 | Epitope |
| S1D12 | NNAVG | GCSSDGTCYYNSA | GHYSIYGYDYLGTIDY | SGSSSNVGGGNSVG | DTNSRPS | VTGDSTTHDDL | 337-355 |
| | (SEQ ID | LKS | (SEQ ID NO: 21) | (SEQ ID NO: 24) | (SEQ ID | (SEQ ID | |
| | NO: 16) | (SEQ ID NO: 18) | | | NO: 26) | NO: 29) | |
| S2C1 | NNAVG | GCSSDGTCYYNSA | NA | NA | NA | NA | 337-355 |
| | (SEQ ID | LKS | | | | | |
| | NO: 16) | (SEQ ID NO: 18) | | | | | |
| ME12 | SNAVG | GCSSDGKCYHNSA | GFYSIYGYDYSGTIDY | SGSSSNVGGGNSVG | NTNSRPS | VTGDSSTHDDL | 337-355 |
| | (SEQ ID | LKS | (SEQ ID NO: 22) | (SEQ ID NO: 24) | (SEQ ID | (SEQ ID | |
| | NO: 17) | (SEQ ID NO: 19) | | | NO: 27) | NO: 30) | |
| NS3D9 | SNAVG | GCSSDGKCYYNSA | GYYPVYGYDYLGTIDY | SGSSSNV-GRNDVA | GTTSRPS | ASGDSSAINDI | 337-355 |
| | (SEQ ID | LKS | (SEQ ID NO: 23) | (SEQ ID NO: 25) | (SEQ ID | (SEQ ID | |
| | NO: 17) | (SEQ ID NO: 20) | | | NO: 28) | NO: 31) | |

The CDRs specified herein are defined according to Kabat. The skilled person is aware that other methods for identifying CDRs are available, such as Chothia and Martin. The use of an alternative method to define CDRs may on occasion alter the residues defined as belonging to one or more CDRs. For example, alternative CDR definitions for S1D12 according to Chothia and Martin are shown in FIG. 1. Any alternative CDR definitions for the specific binding molecule sequences disclosed herein fall within the scope of the invention.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises a VHCDR1 amino acid sequence set forth in table 1;
VHCDR2 comprises a VHCDR2 amino acid sequence set forth in table 1;
VHCDR3 comprises a VHCDR3 amino acid sequence set forth in table 1;
VLCDR1 comprises a VLCDR1 amino acid sequence set forth in table 1;
VLCDR2 comprises a VLCDR2 amino acid sequence set forth in table 1; and
VLCDR3 comprises a VLCDR3 amino acid sequence set forth in table 1;
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 337 to 355 of SEQ ID NO: 1.

The specific binding molecule may bind to a polypeptide or protein molecule comprising an amino acid sequence comprising residues 337 to 355 of SEQ ID NO: 1 with a K_{D} of less than around 500 pM. The K_{D} may be less than around 400 pM, less than around 300 pM, less than around 200 pM or less than around 150 pM. The K_{D} may preferably be for binding to SEQ ID NO: 1 or to SEQ ID NO: 5. The K_{D} for binding to SEQ ID NO: 1 may be around 50 pM to around 150 pM. The K_{D} for binding to SEQ ID NO: 1 may be around 101 pM or 122 pM, optionally wherein the specific binding molecule comprises the CDRs of S1D12. The K_{D} for binding to SEQ ID NO: 5 may be around 300 pM to around 400 pM. The K_{D} for binding to SEQ ID NO: 5 may be around 344 pM, optionally wherein the specific binding molecule comprises the CDRs of S1D12.

The specific binding molecule may comprise an amino acid sequence having at least 70%, at least 75%, at least 80%, at least 90%, at least 95% or at least 99% identity to SEQ ID NO: 32 wherein the specific binding molecule binds to a polypeptide or protein molecule comprising an amino acid sequence comprising residues 337 to 355 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 30. The CDRs may be 100% identical to the CDRs of SEQ ID NO: 32. The specific binding molecule may comprise an amino acid sequence having at least 70%, at least 75%, at least 80%, at least 90%, at least 95% or at least 99% identity to SEQ ID NO: 30 wherein CDRs are 100% identical to the CDRs of SEQ ID NO: 32. The specific binding molecule may comprise the amino acid sequence of SEQ ID NO: 32.

(S1D12 amino acid sequence)
SEQ ID NO: 32
QVQLQESGPSLVKPSQTLSLTCTVSGFSLNNNAVGWVRQAPGKVPESLV
GCSSDGTCYYNSALKSRLDITRDTSKNQISLSLSSVTTDDAAVYYCTRG
HYSIYGYDYLGTIDYWGPGLLVTVSSEGKSSGASGESKVDDQAVLTQPS
SVSGSLGQRVSITCSGSSSNVGGGNSVGWYQHLPGSGLKTIIYDTNSRP
SGVPDRFSGSRSGNTATLTINSLQAEDEGDYYCVTGDSTTHDDLVGSGT
RLTVLG

The epitope of the specific binding molecule may be within an amino acid sequence comprising residues 367 to 379 of SEQ ID NO: 1. Accordingly, the epitope may be within the amino acid sequence of SEQ ID NO: 33 (GNKKIETHKLTFR).

The epitope may be within an amino acid sequence comprising residues 367 to 379 of SEQ ID NO: 1. This epitope may be bound by the CDRs of the specific binding molecule referred to as “S1G2” herein.

The epitope may comprise the amino acid sequence of SEQ ID NO: 33 (GNKKIETHKLTFR). The epitope may comprise an amino acid sequence having at least 70%, at least 75%, at least 80%, at least 90%, at least 95% or at least 99% identity to SEQ ID NO: 33 (GNKKIETHKLTFR). Critical residues of the epitope may be residues 370 (K) and/or 374 (H) (numbering according to SEQ ID NO: 1). The epitope may comprise the amino acid sequence of SEQ ID NO: 34 (XXXKXXXHXXXXX, wherein X is any amino acid). The epitope may comprise the amino acid sequence of SEQ ID NO: 33, wherein any one or more residue other than residue number 370 (K) and/or 374 (H) is replaced by a non-conservative amino acid substitution (numbering according to SEQ ID NO: 1). The epitope may comprise the amino acid sequence of SEQ ID NO: 33, wherein any one or more residue other than residue number 370 (K) and/or 374 (H) is replaced by a conservative amino acid substitution (numbering according to SEQ ID NO: 1). The epitope may comprise the amino acid sequence of SEQ ID NO: 33, wherein any one or more residue other than residue number 370 (K) and/or 374 (H) is replaced by a conservative amino acid substitution (numbering according to SEQ ID NO: 1) and any one or more residue other than residue number 370 (K) and/or 374 (H) is replaced by a non-conservative amino acid substitution (numbering according to SEQ ID NO: 1).

The epitope may consist of the amino acid sequence of SEQ ID NO: 33 (GNKKIETHKLTFR). The epitope may consist of an amino acid sequence having at least 70%, at least 75%, at least 80%, at least 90%, at least 95% or at least 99% identity to SEQ ID NO: 33 (GNKKIETHKLTFR).

The specific binding molecule may bind to a polypeptide or protein molecule comprising an amino acid sequence comprising residues 367 to 379 of SEQ ID NO: 1. Said specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 35 (S/T N/Y S/A/Y V G);
VHCDR2 comprises the sequence set forth in SEQ ID NO: 36 (G/S/N I/V D/Y T/S D/T G E/Y/D/R E/T/A G/Y/F Y/F N P A/V L N/K S);
VHCDR3 comprises the sequence set forth in SEQ ID NO: 37 (S/T Y/V/A R/N A/T/G/S D/-G/-L/Y/F/-A/-Y/H G/P Y/D V Q/Y A/Y I D/E Y/R/K) or SEQ ID NO: 265 (GSYYHGGGNGMVDFFDY);
VLCDR1 comprises the sequence set forth in SEQ ID NO: 38 (S G S/R F/Y/D I/L/V G/S I/S/R S S/R/A/G V G) or in SEQ ID NO: 39 (SGSSSNVGYGNYVG)
VLCDR2 comprises the sequence set forth in SEQ ID NO: 40 (A/D S/A D/S/T G/S R P/A S);
VLCDR3 comprises the sequence set forth in SEQ ID NO: 41 (G/S S/I/V S/F/Y/T D/G/A/Q R/P/-T/-P/Q/D/G Y/R/H/N T/N G/Y V/I/L);
or for each CDR sequence, an amino acid sequence with
   (i) at least 85% identity thereto, and/or
   (ii) one, two, or three amino acid substitutions relative thereto.

Said sequence identity is at least about 85% sequence identity and may therefore be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity. Preferably said sequence identity is at least 90% or at least 95%.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG), SEQ ID NO: 17 (SNAVG), SEQ ID NO: 44 (SYYVG), or SEQ ID NO: 45 (TNSVG);
VHCDR2 comprises the sequence set forth in SEQ ID NO: 46 (GIDTDGEEGYNPALNS), SEQ ID NO: 47 (SVDSDGYTYYNPALKS), SEQ ID NO: 48 (GIDSDGEEGYNPALNS), SEQ ID NO: 49 (GIDSDGEEGYNPALKS), SEQ ID NO: 50 (SVDSDGDTYYNPALKS), SEQ ID NO: 51 (GIDTDGEEGYNPALKS), SEQ ID NO: 52 (NIYSTGRAFYNPALKS), or SEQ ID NO: 53 (GIDTDGEEGFNPVLKS);
VHCDR3 comprises the sequence set forth in SEQ ID NO: 54 (SYRADGLAYGYVQAIDY), SEQ ID NO: 55 (SYRTDGLAYGYVQAIDY), SEQ ID NO: 56 (SVNGHPDVYYIDR), SEQ ID NO: 57 (TYRTDGYAYGYVQAIDY), SEQ ID NO: 58 (SYRSDGLAYGYVQAIDY), SEQ ID NO: 59 (SANGHPDVYYIDK), SEQ ID NO: 60 (TYRTDGFAYGYVQAIDY), SEQ ID NO: 61 (SYRTDGLAYGYVQAIEY), or SEQ ID NO: 265 (GSYYHGGGNGMVDFFDY);

VLCDR1 comprises the sequence set forth in SEQ ID NO: 63 (SGSFIGISSVG); SEQ ID NO: 64 (SGSYISSSRVG); SEQ ID NO: 65 (SGSDLGSSRVG); SEQ ID NO: 66 (SGSYIGSSAVG); SEQ ID NO: 67 (SGRFIGISSVG); SEQ ID NO: 68 (SGSYIGSSGVG); or SEQ ID NO: 69 (SGSYVSRSRVG);
VLCDR2 comprises the sequence set forth in SEQ ID NO: 70 (ASDGRPS); SEQ ID NO: 71 (DSSSRPS); or SEQ ID NO: 72 (AATSRAS);
VLCDR3 comprises the sequence set forth in SEQ ID NO: 73 (GSSDRTPYTGV); SEQ ID NO: 74 (GSSDRTQYTGV); SEQ ID NO: 75 (GVFGDRNYI); SEQ ID NO: 76 (GIFGDRNYI); SEQ ID NO: 77 (GSTAPTPHTGV); SEQ ID NO: 78 (SSYQRGNTGV); SEQ ID NO: 79 (GSSDRTQYTGL); or SEQ ID NO: 80 (GIYGDRNYI);
or for each CDR sequence, an amino acid sequence with
   (i) at least 85% identity thereto, and/or
   (ii) one, two, or three amino acid substitutions relative thereto,
   wherein the specific binding molecule binds to a polypeptide or protein molecule comprising an amino acid sequence comprising residues 367 to 379 of SEQ ID NO: 1.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 54 (SYRADGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS);
VLCDR3 comprises the sequence set forth in SEQ ID NO: 73 (GSSDRTPYTGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 367 to 379 of SEQ ID NO: 1. The specific binding molecule comprising CDRs having 100% identity to those given above is referred to as “S1G2” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 54 (SYRADGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS); and
VLCDR3 comprises the sequence set forth in SEQ ID NO: 73 (GSSDRTPYTGV).

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 447 (QVQLQESGPSLVKPSQTLSLTCTVSGFSLT);
VHFR2 comprises the sequence set forth in SEQ ID NO: 448 (WVRQAPGKAPEWVA);
VHFR3 comprises the sequence set forth in SEQ ID NO: 449 (RLSITRDTSKSQVSLSLSSVTSEDTAVYYCGR);
VHFR4 comprises the sequence set forth in SEQ ID NO: 450 (WGPGLLVTVSS);
VLFR1 comprises the sequence set forth in SEQ ID NO: 451 (QAVVTQPSSVSGSLGQRVSITC);
VLFR2 comprises the sequence set forth in SEQ ID NO: 452 (WFQQLPGSGLRTIIV);
VLFR3 comprises the sequence set forth in SEQ ID NO: 453 (GVPDRFSMSKSGNTATLTISSLQAEDEADYFC);
VLFR4 comprises the sequence set forth in SEQ ID NO: 454 (FGSGTRLTVLG);
or for each FR sequence, an amino acid sequence with
   (i) at least 50% identity thereto, and/or
   (ii) one, two, three, four or five amino acid substitutions relative thereto.

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 447 (QVQLQESGPSLVKPSQTLSLTCTVSGFSLT);
VHFR2 comprises the sequence set forth in SEQ ID NO: 448 (WVRQAPGKAPEWVA);
VHFR3 comprises the sequence set forth in SEQ ID NO: 449 (RLSITRDTSKSQVSLSLSSVTSEDTAVYYCGR);
VHFR4 comprises the sequence set forth in SEQ ID NO: 450 (WGPGLLVTVSS);
VLFR1 comprises the sequence set forth in SEQ ID NO: 451 (QAVVTQPSSVSGSLGQRVSITC);
VLFR2 comprises the sequence set forth in SEQ ID NO: 452 (WFQQLPGSGLRTIIV);
VLFR3 comprises the sequence set forth in SEQ ID NO: 453 (GVPDRFSMSKSGNTATLTISSLQAEDEADYFC);
VLFR4 comprises the sequence set forth in SEQ ID NO: 454 (FGSGTRLTVLG);
or for each FR sequence, an amino acid sequence with
   (i) at least 50% identity thereto, and/or
   (ii) one, two, three, four or five amino acid substitutions relative thereto,
wherein the specific binding molecule binds to a polypeptide or protein molecule comprising an amino acid sequence comprising residues 367 to 379 of SEQ ID NO: 1. A specific binding molecule comprising FRs having 100% identity to those given above is referred to as “S1G2” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 447 (QVQLQESGPSLVKPSQTLSLTCTVSGFSLT);
   VHFR2 comprises the sequence set forth in SEQ ID NO: 448 (WVRQAPGKAPEWVA);
   VHFR3 comprises the sequence set forth in SEQ ID NO: 449 (RLSITRDTSKSQVSLSLSSVTSEDTAVYYCGR);
   VHFR4 comprises the sequence set forth in SEQ ID NO: 450 (WGPGLLVTVSS);
   VLFR1 comprises the sequence set forth in SEQ ID NO: 451 (QAVVTQPSSVSGSLGQRVSITC);
   VLFR2 comprises the sequence set forth in SEQ ID NO: 452 (WFQQLPGSGLRTIIV);
   VLFR3 the set forth in comprises sequence SEQ ID NO: 453 (GVPDRFSMSKSGNTATLTISSLQAEDEADYFC);
   VLFR4 comprises the sequence set forth in SEQ ID NO: 454 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 42 (SNSVG);
   VHCDR2 comprises the sequence set forth in SEQ ID NO: 46 (GIDTDGEEGYNPALNS);
   VHCDR3 comprises the sequence set forth in SEQ ID NO: 54 (SYRADGLAYGYVQAIDY);
   VLCDR1 comprises the sequence set forth in SEQ ID NO: 63 (SGSFIGISSVG);
   VLCDR2 comprises the sequence set forth in SEQ ID NO: 70 (ASDGRPS); and
   VLCDR3 comprises the sequence set forth in SEQ ID NO: 73 (GSSDRTPYTGV).
   or for each CDR sequence, an amino acid sequence with
      (i) at least 85% identity thereto, and/or
      (ii) one, two, or three amino acid substitutions relative thereto
   wherein the specific binding molecule binds to a polypeptide or protein molecule comprising an amino acid sequence comprising residues 367 to 379 of SEQ ID NO: 1. The specific binding molecule comprising FRs and CDRs having 100% identity to those given above is referred to as “S1G2” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 447 (QVQLQESGPSLVKPSQTLSLTCTVSGFSLT);
   VHFR2 comprises the sequence set forth in SEQ ID NO: 448 (WVRQAPGKAPEWVA);
   VHFR3 comprises the sequence set forth in SEQ ID NO: 449 (RLSITRDTSKSQVSLSLSSVTSEDTAVYYCGR);
   VHFR4 comprises the sequence set forth in SEQ ID NO: 450 (WGPGLLVTVSS);
   VLFR1 comprises the sequence set forth in SEQ ID NO: 451 (QAVVTQPSSVSGSLGQRVSITC);
   VLFR2 comprises the sequence set forth in SEQ ID NO: 452 (WFQQLPGSGLRTIIV);
   VLFR3 comprises the sequence set forth in SEQ ID NO: 453 (GVPDRFSMSKSGNTATLTISSLQAEDEADYFC);
   VLFR4 comprises the sequence set forth in SEQ ID NO: 454 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 42 (SNSVG);
   VHCDR2 comprises the sequence set forth in SEQ ID NO: 46 (GIDTDGEEGYNPALNS);
   VHCDR3 comprises the sequence set forth in SEQ ID NO: 54 (SYRADGLAYGYVQAIDY);
   VLCDR1 comprises the sequence set forth in SEQ ID NO: 63 (SGSFIGISSVG);
   VLCDR2 comprises the sequence set forth in SEQ ID NO: 70 (ASDGRPS); and
   VLCDR3 comprises the sequence set forth in SEQ ID NO: 73 (GSSDRTPYTGV).
   wherein the specific binding molecule binds to a polypeptide or protein molecule comprising an amino acid sequence comprising residues 367 to 379 of SEQ ID NO: 1. The specific binding molecule comprising FRs and CDRs having 100% identity to those given above is referred to as “S1G2” herein.

The specific binding molecule may comprise:
(a) A VH domain comprising the sequence set forth in SEQ ID NO: 455 (QVQLQESGPSLVKPSQTLSLTCTVSGFSLTSNSVGWVRQAPGKAPEWVAGIDTDGEEG YNPALNSRLSITRDTSKSQVSLSLSSVTSEDTAVYYCGRSYRADGLAYGYVQAIDYWGPG LLVTVSS); and/or
(b) a VL domain comprising the sequence set forth in SEQ ID NO: 456 (QAVVTQPSSVSGSLGQRVSITCSGSFIGISSVGWFQQLPGSGLRTIIVASDGRPSGVPDR FSMSKSGNTATLTISSLQAEDEADYFCGSSDRTPYTGVFGSGTRLTVLG);
or a humanized variant thereof.

The specific binding molecule may comprise:
(a) A heavy chain comprising the sequence set forth in SEQ ID NO: 457 (QVQLQESGPSLVKPSQTLSLTCTVSGFSLTSNSVGWVRQAPGKAPEWVAGIDTDGEEG YNPALNSRLSITRDTSKSQVSLSLSSVTSEDTAVYYCGRSYRADGLAYGYVQAIDYWGPG LLVTVSSAKTTAPSVYPLAPVCGDTTGSSVTLGCLVKGYFPEPVTLTWNSGSLSSGVHTF PAVLQSDLYTLSSSVTVTSSTWPSQSITCNVAHPASSTKVDKKIEPRGPTIKPCPPCKCPA PNLLGGPSVFIFPPKIKDVLMISLSPIVTCVVVDVSEDDPDVQISWFVNNVEVHTAQTQTHR EDYNSTLRVVSALPIQHQDWMSGKEFKCKVNNKDLPAPIERTISKPKGSVRAPQVYVLPP PEEEMTKKQVTLTCMVTDFMPEDIYVEWTNNGKTELNYKNTEPVLDSDGSYFMYSKLRV EKKNWVERNSYSCSVVHEGLHNHHTTKSFSRTPGK); and/or
(b) a light chain comprising the sequence set forth in SEQ ID NO: 458 (QAVVTQPSSVSGSLGQRVSITCSGSFIGISSVGWFQQLPGSGLRTIIVASDGRPSGVPDR FSMSKSGNTATLTISSLQAEDEADYFCGSSDRTPYTGVFGSGTRLTVLGGQPKSSPSVTL FPPSSEELETNKATLVCTITDFYPGVVTVDWKVDGTPVTQGMETTQPSKQSNNKYMASS YLTLTARAWERHSSYSCQVTHEGHTVEKSLSRADCS);
or a humanized variant thereof.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 55 (SYRTDGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS);
VLCDR3 comprises the sequence set forth in SEQ ID NO: 74 (GSSDRTQYTGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 367 to 379 of SEQ ID NO: 1. The specific binding molecule comprising CDRs having 100% identity to those given above is referred to as “S1B1” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 55 (SYRTDGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS);
VLCDR3 comprises the sequence set forth in SEQ ID NO: 74 (GSSDRTQYTGV).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 48 (GIDSDGEEGYNPALNS);
VHCDR3 comprises the sequence set forth in SEQ ID NO: 57 (TYRTDGYAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS);
VLCDR3 comprises the sequence set forth in SEQ ID NO: 74 (GSSDRTQYTGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 367 to 379 of SEQ ID NO: 1. The specific binding molecule comprising CDRs having 100% identity to those given above is referred to as “S1D9” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 48 (GIDSDGEEGYNPALNS);
VHCDR3 comprises the sequence set forth in SEQ ID NO: 57 (TYRTDGYAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS);
VLCDR3 comprises the sequence set forth in SEQ ID NO: 74 (GSSDRTQYTGV).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 54 (SYRADGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS);
VLCDR3 comprises the sequence set forth in SEQ ID NO: 74 (GSSDRTQYTGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 367 to 379 of SEQ ID NO: 1. The specific binding molecule comprising CDRs having 100% identity to those given above is referred to as “S1F4” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 54 (SYRADGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS);
VLCDR3 comprises the sequence set forth in SEQ ID NO: 74 (GSSDRTQYTGV).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 54 (SYRADGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS);
VLCDR3 comprises the sequence set forth in SEQ ID NO: 74 (GSSDRTQYTGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 367 to 379 of SEQ ID NO: 1. The specific binding molecule comprising CDRs having 100% identity to those given above is referred to as “S1G10” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 54 (SYRADGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS);
VLCDR3 comprises the sequence set forth in SEQ ID NO: 74 (GSSDRTQYTGV).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 51 (GIDTDGEEGYNPALKS);
VHCDR3 comprises the sequence set forth in SEQ ID NO: 60 (TYRTDGFAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS);
VLCDR3 comprises the sequence set forth in SEQ ID NO: 74 (GSSDRTQYTGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 367 to 379 of SEQ ID NO: 1. The specific binding molecule comprising CDRs having 100% identity to those given above is referred to as “S2C6” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 51 (GIDTDGEEGYNPALKS);
VHCDR3 comprises the sequence set forth in SEQ ID NO: 60 (TYRTDGFAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS);
VLCDR3 comprises the sequence set forth in SEQ ID NO: 74 (GSSDRTQYTGV).

The specific binding molecule may comprise the CDR sequences of a clone set out in Table 2 below. The epitope may be within residues 367 to 379 of SEQ ID NO: 1.

TABLE 2

| | VH | | | | | | |
|---|---|---|---|---|---|---|---|
| Clone | | | | VL | | | |
| name | CDR1 | CDR2 | CDR3 | CDR1 | CDR2 | CDR3 | Epitope |
| S1B1 | SNSVG | GIDTDGEEGYNPALNS | SYRTDGLAYGYVQAIDY | SGSFIGISSVG (SEQ | ASDGRPS | GSSDRTQYTGV (SEQ | 367-379 |
| | (SEQ ID NO: | (SEQ ID NO: 46) | (SEQ ID NO: 55) | ID NO: 63) | (SEQ ID NO: | ID NO: 74) | |
| | 42) | | | | 70) | | |
| S1D2 | SNAVG | SVDSDGYTYYNPALKS | SVNG----HPDVYYIDR | SGSYISSSRVG (SEQ | DSSSRPS | GVFG--DRNYI (SEQ ID | 367-379 |
| | (SEQ ID NO: | (SEQ ID NO: 47) | (SEQ ID NO: 56) | ID NO: 64) | (SEQ ID NO: | NO: 75) | |
| | 17) | | | | 71) | | |
| S1D9 | SNSVG | GIDSDGEEGYNPALNS | TYRTDGYAYGYVQAIDY | SGSFIGISSVG (SEQ | ASDGRPS | GSSDRTQYTGV (SEQ ID | 367-379 |
| | (SEQ ID NO: | (SEQ ID NO: 48) | (SEQ ID NO: 57) | ID NO: 63) | (SEQ ID NO: | NO: 74) | |
| | 42) | | | | 70) | | |
| S1F4 | SNSVG | GIDTDGEEGYNPALNS | SYRADGLAYGYVQAIDY | SGSFIGISSVG (SEQ | ASDGRPS | GSSDRTQYTGV (SEQ ID | 367-379 |
| | (SEQ ID NO: | (SEQ ID NO: 46) | (SEQ ID NO: 54) | ID NO: 63) | (SEQ ID NO: | NO: 74) | |
| | 42) | | | | 70) | | |
| S1G2 | SNSVG | GIDTDGEEGYNPALNS | SYRADGLAYGYVQAIDY | SGSFIGISSVG (SEQ | ASDGRPS | GSSDRTPYTGV (SEQ ID | 367-379 |
| | (SEQ ID NO: | (SEQ ID NO: 46) | (SEQ ID NO: 54) | ID NO: 63) | (SEQ ID NO: | NO: 73) | |
| | 42) | | | | 70) | | |
| S1G10 | SNSVG | GIDTDGEEGYNPALNS | SYRADGLAYGYVQAIDY | SGSFIGISSVG (SEQ | ASDGRPS | GSSDRTQYTGV (SEQ ID | 367-379 |
| | (SEQ ID NO: | (SEQ ID NO: 46) | (SEQ ID NO: 54) | ID NO: 63) | (SEQ ID NO: | NO: 74) | |
| | 42) | | | | 70) | | |
| S1H6 | SNAVG | SVDSDGYTYYNPALKS | SVNG----HPDVYYIDR | SGSDLGSSRVG | DSSSRPS | GIFG--DRNYI (SEQ ID | 367-379 |
| | (SEQ ID NO: | (SEQ ID NO: 47) | (SEQ ID NO: 56) | (SEQ ID NO: 65) | (SEQ ID NO: | NO: 76) | |
| | 17) | | | | 71) | | |
| S1H9 | SNSVG | GIDSDGEEGYNPALKS | SYRSDGLAYGYVQAIDY | SGSFIGISSVG (SEQ | ASDGRPS | GSSDRTQYTGV (SEQ ID | 367-379 |
| | (SEQ ID NO: | (SEQ ID NO: 49) | (SEQ ID NO: 58) | ID NO: 63) | (SEQ ID NO: | NO: 74) | |
| | 42) | | | | 70) | | |
| S2C3 | SNAVG | SVDSDGDTYYNPALKS | SANG----HPDVYYIDK | SGSYISSSRVG (SEQ | DSSSRPS | GIFG--DRNYI (9) | 367-379 |
| | (SEQ ID NO: | (SEQ ID NO: 50) | (SEQ ID NO: 59) | ID NO: 64) | (SEQ ID NO: | | |
| | 17) | | | | 71) | | |
| S2C6 | SNSVG | GIDTDGEEGYNPALKS | TYRTDGFAYGYVQAIDY | SGSFIGISSVG (SEQ | ASDGRPS | GSSDRTQYTGV (SEQ ID | 367-379 |
| | (SEQ ID NO: | (SEQ ID NO: 51) | (SEQ ID NO 60) | ID NO: 63) | (SEQ ID NO: | NO: 74) | |
| | 42) | | | | 70) | | |
| S2D1 | SNSVG | GIDTDGEEGYNPALNS | SYRTDGLAYGYVQAIDY | SGSYIGSSAVG | ASDGRPS | Incomplete | 367-379 |
| | (SEQ ID NO: | (SEQ ID NO: 46) | (SEQ ID NO: 55) | (SEQ ID NO: 66) | (SEQ ID NO: | | |
| | 42) | | | | 70) | | |
| S2D4 | SNSVG | GIDTDGEEGYNPALNS | SYRTDGLAYGYVQAIEY | SGRFIGISSVG (SEQ | ASDGRPS | GSTAPTPHTGV (SEQ ID | 367-379 |
| | (SEQ ID NO: | (SEQ ID NO: 46) | (SEQ ID NO: 61) | ID NO: 67) | (SEQ ID NO: | NO: 77) | |
| | 42) | | | | 70) | | |
| CA9 | SNSVG | GIDTDGEEGYNPALNS | SYRSDGLAYGYVQAIDY | SGSFIGISSVG (SEQ | ASDGRPS | GSSDRTQYTGV (SEQ ID | 367-379 |
| | (SEQ ID NO: | (SEQ ID NO: 46) | (SEQ ID NO: 58) | ID NO: 63) | (SEQ ID NO: | NO: 74) | |
| | 42) | | | | 70) | | |
| CA12 | SYYVG | NIYSTGRAFYNPALKS | GSYYHGGGNGMVDFFDY | SGSSSNVGYGNYVG | AATSRAS | SSYQR-GNTGV (SEQ ID | 367-379 |
| | (SEQ ID NO: | (SEQ ID NO: 52) | (SEQ ID NO: 265) | (14) | (SEQ ID NO: | NO: 78) | |
| | 44) | | | | 72) | | |
| CB2 | TNSVG | GIDTDGEEGFNPVLKS | SYRTDGLAYGYVQAIDY | SGSYIGSSGVG | ASDGRPS | GSSDRTQYTGL (SEQ ID | 367-379 |
| | (SEQ ID NO: | (SEQ ID NO: 53) | (SEQ ID NO: 55) | (SEQ ID NO 68) | (SEQ ID NO: | NO: 79) | |
| | 45) | | | | 70) | | |
| CC12 | SNSVG | GIDSDGEEGYNPALNS | SYRADGLAYGYVQAIDY | SGRFIGISSVG (SEQ | ASDGRPS | GSSDRTQYTGV (SEQ ID | 367-379 |
| | (SEQ ID NO: | (SEQ ID NO: 48) | (SEQ ID NO: 54) | ID NO: 67) | (SEQ ID NO: | NO: 74) | |
| | 42) | | | | 70) | | |
| MC5 | SNAVG | SVDSDGDTYYNPALKS | SVNG----HPDVYYIDR | SGSYVSRSRVG | DSSSRPS | GIYG--DRNYI (SEQ ID | 367-379 |
| | (SEQ ID NO: | (SEQ ID NO: 50) | (SEQ ID NO: 56) | (SEQ ID NO: 69) | (SEQ ID NO: | NO: 80) | |
| | 17) | | | | 71) | | |
| MD12 | SNAVG | SVDSDGYTYYNPALKS | SVNG----HPDVYYIDR | SGSYISSSRVG (SEQ | DSSSRPS | GVFG--DRNYI (SEQ ID | 367-379 |
| | (SEQ ID NO: | (SEQ ID NO: 47) | (SEQ ID NO: 56) | ID NO: 64) | (SEQ ID NO: | NO: 75) | |
| | 17) | | | | 72) | | |

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises a VHCDR1 amino acid sequence set forth in table 2;
VHCDR2 comprises a VHCDR2 amino acid sequence set forth in table 2;
VHCDR3 comprises a VHCDR3 amino acid sequence set forth in table 2;
VLCDR1 comprises a VLCDR1 amino acid sequence set forth in table 2;
VLCDR2 comprises a VLCDR2 amino acid sequence set forth in table 2; and
VLCDR3 comprises a VLCDR3 amino acid sequence set forth in table 2;
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 367 to 379 of SEQ ID NO: 1.

The specific binding molecule may bind to a polypeptide or protein molecule comprising an amino acid sequence comprising residues 367 to 379 of SEQ ID NO: 1 with a K_{D} of less than around 500 pM. The K_{D} may be less than around 400 pM, less than around 300 pM, or less than around 200 pM. The K_{D} may preferably be for binding to SEQ ID NO: 1 or to SEQ ID NO: 5. The K_{D} for binding to SEQ ID NO: 1 may be around 100 pM to around 200 pM. The K_{D} for binding to SEQ ID NO: 1 may be around 140 pM or 170 pM, optionally wherein the specific binding molecule comprises the CDRs of S1G2. The K_{D} for binding to SEQ ID NO: 5 may be around 400 pM to around 500 pM. The K_{D} for binding to SEQ ID NO: 5 may be around 447 pM, optionally wherein the specific binding molecule comprises the CDRs of S1G2.

The specific binding molecule may comprise an amino acid sequence having at least 70%, at least 75%, at least 80%, at least 90%, at least 95% or at least 99% identity to SEQ ID NO: 81 wherein the specific binding molecule binds to a polypeptide or protein molecule comprising an amino acid sequence comprising residues 367 to 379 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 81. The CDRs may be 100% identical to the CDRs of SEQ ID NO: 81. The specific binding molecule may comprise an amino acid sequence having at least 70%, at least 75%, at least 80%, at least 90%, at least 95% or at least 99% identity to SEQ ID NO: 81 wherein CDRs are 100% identical to the CDRs of SEQ ID NO: 81. The specific binding molecule may comprise the amino acid sequence of SEQ ID NO: 81.

(S1G2 amino acid sequence)
SEQ ID NO: 81
QVQLQESGPSLVKPSQTLSLTCTVSGFSLTSNSVGWVRQAPGKAPEWVA
GIDTDGEEGYNPALNSRLSITRDTSKSQVSLSLSSVTSEDTAVYYCGRS
YRADGLAYGYVQAIDYWGPGLLVTVSSEGKSSGASGESKVDDQAVVTQP
SSVSGSLGQRVSITCSGSFIGISSVGWFQQLPGSGLRTIIVASDGRPSG
VPDRFSMSKSGNTATLTISSLQAEDEADYFCGSSDRTPYTGVFGSGTRL
TVLG

The specific binding molecule may comprise an amino acid sequence having at least 70%, at least 75%, at least 80%, at least 90%, at least 95% or at least 99% identity to SEQ ID NO: 412 wherein the specific binding molecule binds to a polypeptide or protein molecule comprising an amino acid sequence comprising residues 367 to 379 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 412. The CDRs may be 100% identical to the CDRs of SEQ ID NO: 412. The specific binding molecule may comprise an amino acid sequence having at least 70%, at least 75%, at least 80%, at least 90%, at least 95% or at least 99% identity to SEQ ID NO: 412 wherein CDRs are 100% identical to the CDRs of SEQ ID NO: 412. The specific binding molecule may comprise the amino acid sequence of SEQ ID NO: 412.

(S1B1 amino acid sequence)
SEQ ID NO: 412
QVQLQESGPSLVKPSQTLSLTCTVSGFSLSSNSVGWVRQAPGKAPEWVA
GIDTDGEEGYNPALNSRLSITRDTSKSQVSLSLSSVTSEDTAVYYCVRS
YRTDGLAYGYVQAIDYWGPGLLVTVSSEGKSSGASGESKVDDQAVLTQP
SSVSGSLGQRVSITCSGSFIGISSVGWFQQLPGSGLRTVIVASDGRPSG
VPDRFSNSKSGNTATLTISSLQAEDEADYFCGSSDRTQYTGVFGSGTRL
TVLG

The specific binding molecule may comprise an amino acid sequence having at least 70%, at least 75%, at least 80%, at least 90%, at least 95% or at least 99% identity to SEQ ID NO: 413 wherein the specific binding molecule binds to a polypeptide or protein molecule comprising an amino acid sequence comprising residues 367 to 379 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 413. The CDRs may be 100% identical to the CDRs of SEQ ID NO: 413. The specific binding molecule may comprise an amino acid sequence having at least 70%, at least 75%, at least 80%, at least 90%, at least 95% or at least 99% identity to SEQ ID NO: 413 wherein CDRs are 100% identical to the CDRs of SEQ ID NO: 413. The specific binding molecule may comprise the amino acid sequence of SEQ ID NO: 413.

(S1D9 amino acid sequence)
SEQ ID NO: 413
QVQLQESGPSLVKPSQTLSLTCTVSGFSLTSNSVGWVRQAPGKAPEWVA
GIDSDGEEGYNPALNSRLSITRDTSKNQVSLSLSRVTSEDTAVYYCGRT
YRTDGYAYGYVQAIDYWGPGLLVTVSSEGKSSGASGESKVDDRVMLTQP
PSVSGSPGQTVSITCSGSFIGISSVGWFQQLPGSGLRTVIFASDGRPSG
VPDRFSNSKSGNTATLTISSLQAEDEADYFCGSSDRTQYTGVFGSGTRL
TVLS

The specific binding molecule may comprise an amino acid sequence having at least 70%, at least 75%, at least 80%, at least 90%, at least 95% or at least 99% identity to SEQ ID NO: 414 wherein the specific binding molecule binds to a polypeptide or protein molecule comprising an amino acid sequence comprising residues 367 to 379 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 414. The CDRs may be 100% identical to the CDRs of SEQ ID NO: 414. The specific binding molecule may comprise an amino acid sequence having at least 70%, at least 75%, at least 80%, at least 90%, at least 95% or at least 99% identity to SEQ ID NO: 414 wherein CDRs are 100% identical to the CDRs of SEQ ID NO: 414. The specific binding molecule may comprise the amino acid sequence of SEQ ID NO: 414.

(S1F4 amino acid sequence)
SEQ ID NO: 414
QVQLQESGPSLVKPSQTLSLTCTVSGFSLSSNSVGWVRQAPGKAPEWVA
GIDTDGEEGYNPALNSRLSITRDTSKSQVSLSLSSVTSEDTAVYYCVRS
YRADGLAYGYVQAIDYWGPGLLLTISSEGKSSGASGESKVDDQAVVTQP
SSVSGSLGQRVSITCSGSFIGISSVGWFQQLPGSGLRTVIVASDGRPSG
VPDRFSNSKSGNTATLTISSLQAEDEADYFCGSSDRTQYTGVFGSGTRL
TVLG

The specific binding molecule may comprise an amino acid sequence having at least 70%, at least 75%, at least 80%, at least 90%, at least 95% or at least 99% identity to SEQ ID NO: 415 wherein the specific binding molecule binds to a polypeptide or protein molecule comprising an amino acid sequence comprising residues 367 to 379 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 415. The CDRs may be 100% identical to the CDRs of SEQ ID NO: 415. The specific binding molecule may comprise an amino acid sequence having at least 70%, at least 75%, at least 80%, at least 90%, at least 95% or at least 99% identity to SEQ ID NO: 415 wherein CDRs are 100% identical to the CDRs of SEQ ID NO: 415. The specific binding molecule may comprise the amino acid sequence of SEQ ID NO: 415.

(S1G10 amino acid sequence)
SEQ ID NO: 415
QVQLQESGPSLVKPSQTLSLTCTVSGFSLTSNSVGWVRQAPGKAPEWVA
GIDTDGEEGYNPALNSRLSITRDTSKSQVSLSLSSVTSEDTAVYYCGRS
YRADGLAYGYVQAIDYWGPGLLVTVSSEGKSSGASGESKVDDQAVLTQP
SSMSGSLGQRVSITCSGSFIGISSVGWFQQLPGSGLRTIIVASDGRPSG
VPDRFSMSKSGNTATLTISSLQAEDEADYFCGSSDRTQYTGVFGSGTRL
TVLG

The specific binding molecule may comprise an amino acid sequence having at least 70%, at least 75%, at least 80%, at least 90%, at least 95% or at least 99% identity to SEQ ID NO: 416 wherein the specific binding molecule binds to a polypeptide or protein molecule comprising an amino acid sequence comprising residues 367 to 379 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 416. The CDRs may be 100% identical to the CDRs of SEQ ID NO: 416. The specific binding molecule may comprise an amino acid sequence having at least 70%, at least 75%, at least 80%, at least 90%, at least 95% or at least 99% identity to SEQ ID NO: 416 wherein CDRs are 100% identical to the CDRs of SEQ ID NO: 416. The specific binding molecule may comprise the amino acid sequence of SEQ ID NO: 416.

(S2C6 amino acid sequence)
SEQ ID NO: 416
QVQLQESGPSLVKPSQTLSLTCTVSGFSLISNSVGWVRQAPGKAPEWVA
GIDTDGEEGYNPALKSQYAASDPDTSKSQVSLSLSSVTSEDTAVYYCGR
TYRTDGFAYGYVQAIDYWGPGLLLTISSEGKSSGASGESKVDDQAVLTQ
PSSVSGSLGQRVSITCSGSFIGISSVGWFQQLPGSGLRTIIVASDGRPS
GVPDRFSMSKSGNTATLTISSLQAEDEADYFCGSSDRTQYTGVFGSGTR
LTVLG

The epitope of the specific binding molecule may be within an amino acid sequence comprising residues 337 to 368 of SEQ ID NO: 1. Accordingly, the epitope may be within the amino acid sequence of SEQ ID NO: 82 (VEVKSEKLDFKDRVQSKIGSLDNITHVPGGGN).

The epitope may be within an amino acid sequence comprising residues 337 to 368 of SEQ ID NO: 1. This epitope may be bound by the CDRs of the specific binding molecule referred to as “NS2A3” herein.

The epitope may comprise the amino acid sequence of SEQ ID NO: 82 (VEVKSEKLDFKDRVQSKIGSLDNITHVPGGGN). The epitope may comprise an amino acid sequence having at least 70%, at least 75%, at least 80%, at least 90%, at least 95% or at least 99% identity to SEQ ID NO: 82 (VEVKSEKLDFKDRVQSKIGSLDNITHVPGGGN).

The epitope may consist of the amino acid sequence of SEQ ID NO: 82 (VEVKSEKLDFKDRVQSKIGSLDNITHVPGGGN). The epitope may consist of an amino acid sequence having at least 70%, at least 75%, at least 80%, at least 90%, at least 95% or at least 99% identity to SEQ ID NO: 82 (VEVKSEKLDFKDRVQSKIGSLDNITHVPGGGN).

The specific binding molecule may bind to a polypeptide or protein molecule comprising an amino acid sequence comprising residues 337 to 368 of SEQ ID NO: 1. Said specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 83 (S Y S V Y)
VHCDR2 comprises the sequence set forth in SEQ ID NO: 84 (I M Y A S G R V D Y N P A L K S)
VHCDR3 comprises the sequence set forth in SEQ ID NO: 85 (G I E N/D)
VLCDR1 comprises the sequence set forth in SEQ ID NO: 86 (R T S/N Q/E S/N V/I N/G/D N/S Y/G L S/A)
VLCDR2 comprises the sequence set forth in SEQ ID NO: 87 (Y A T Y L Y/H T)
VLCDR3 comprises the sequence set forth in SEQ ID NO: 88 (L Q Y D/G/E S/T T P L A/T)
or for each CDR sequence, an amino acid sequence with
   (i) at least 85% identity thereto, and/or
   (ii) one, two, or three amino acid substitutions relative thereto.

Said sequence identity is at least about 85% sequence identity and may therefore be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity. Preferably said sequence identity is at least 90% or at least 95%.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 83 (SYSVY);
VHCDR2 comprises the sequence set forth in SEQ ID NO: 84 (IMYASGRVDYNPALKS);
VHCDR3 comprises the sequence set forth in SEQ ID NO: 89 (GIEN) or SEQ ID NO: 90 (GIED);
VLCDR1 comprises the sequence set forth in SEQ ID NO: 91 (RTSQSVNNYLS), SEQ ID NO: 92 (RTNESVGNYLS), SEQ ID NO: 93 (RTSQNIDNGLA), or SEQ ID NO 94 (RTSQSVGSYLS);
VLCDR2 comprises the sequence set forth in SEQ ID NO: 95 (YATRLYT) or SEQ ID NO: 96 (YATRLHT);
VLCDR3 comprises the sequence set forth in SEQ ID NO: 97 (LQYDSTPLA), SEQ ID NO: 98 (LQYDSTPLT), SEQ ID NO: 99 (LQYESTPLA), or SEQ ID NO: 100 (LQYGTTPLA);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 337 to 368 of SEQ ID NO: 1.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 83 (SYSVY);
VHCDR2 comprises the sequence set forth in SEQ ID NO: 84 (IMYASGRVDYNPALKS);
VHCDR3 comprises the sequence set forth in SEQ ID NO: 89 (GIEN);
VLCDR1 comprises the sequence set forth in SEQ ID NO: 91 (RTSQSVNNYLS);
VLCDR2 comprises the sequence set forth in SEQ ID NO: 95 (YATRLYT);
VLCDR3 comprises the sequence set forth in SEQ ID NO: 97 (LQYDSTPLA);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 337 to 368 of SEQ ID NO: 1. The specific binding molecule comprising CDRs having 100% identity to those given above is referred to as “NS2A3” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 83 (SYSVY);
VHCDR2 comprises the sequence set forth in SEQ ID NO: 84 (IMYASGRVDYNPALKS);
VHCDR3 comprises the sequence set forth in SEQ ID NO: 89 (GIEN);
VLCDR1 comprises the sequence set forth in SEQ ID NO: 91 (RTSQSVNNYLS);
VLCDR2 comprises the sequence set forth in SEQ ID NO: 95 (YATRLYT); and
VLCDR3 comprises the sequence set forth in SEQ ID NO: 97 (LQYDSTPLA).

The specific binding molecule may comprise the CDR sequences of a clone set out in Table 3 below. The epitope may be within residues 337 to 368 of SEQ ID NO:1.

TABLE 3

| Clone | VH | | | VL | | | |
|---|---|---|---|---|---|---|---|
| name | CDR1 | CDR2 | CDR3 | CDR1 | CDR2 | CDR3 | Epitope |
| NS2A3 | SYSVY | IMYASGRVDYNPALKS | GIEN (SEQ ID | RTSQSVNNYLS | YATRLYT | LQYDSTPLA | 337-368 |
| | (SEQ ID | (SEQ ID NO: 84) | NO: 89) | (SEQ ID NO: | (SEQ ID | (SEQ ID NO: | |
| | NO: 83) | | | 91) | NO: 95) | 97) | |
| NS2A8 | SYSVY | IMYASGRVDYNPALKS | GIEN (SEQ ID | RTNESVGNYLS | YATRLHT | LQYGTTPLA | 337-368 |
| | (SEQ ID | (SEQ ID NO: 84) | NO: 89) | (SEQ ID NO: | (SEQ ID | (SEQ ID NO: | |
| | NO: 83) | | | 92) | NO: 96) | 100) | |
| NS2C5 | SYSVY | IMYASGRVDYNPALKS | GIED (SEQ ID | RTSQNIDNGLA | YATRLHT | LQYESTPLA | 337-368 |
| | (SEQ ID | (SEQ ID NO: 84) | NO: 90) | (SEQ | (SEQ ID | (SEQ ID NO: | |
| | NO: 83) | | | ID NO: 93) | NO: 96) | 99) | |
| NS2C8 | SYSVY | IMYASGRVDYNPALKS | GIEN (SEQ ID | RTSQSVNNYLS | YATRLYT | LQYDSTPLA | 337-368 |
| | (SEQ ID | (SEQ ID NO: 84) | NO: 89) | (SEQ | (SEQ ID | (SEQ ID NO: | |
| | NO: 83) | | | ID NO: 91) | NO: 95) | 97) | |
| NS2D3 | SYSVY | IMYASGRVDYNPALKS | GIED (SEQ ID | RTSQSVGSYLS | YATRLHT | LQYDSTPLT | 337-368 |
| | (SEQ ID | (SEQ ID NO: 84) | NO: 89) | (SEQ | (SEQ ID | (SEQ ID NO: | |
| | NO: 83) | | | ID NO: 94) | NO: 96) | 98) | |

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises a VHCDR1 amino acid sequence set forth in table 3;
VHCDR2 comprises a VHCDR2 amino acid sequence set forth in table 3;
VHCDR3 comprises a VHCDR3 amino acid sequence set forth in table 3;
VLCDR1 comprises a VLCDR1 amino acid sequence set forth in table 3;
VLCDR2 comprises a VLCDR2 amino acid sequence set forth in table 3; and
VLCDR3 comprises a VLCDR3 amino acid sequence set forth in table 3;
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 337 to 368 of SEQ ID NO: 1.

The specific binding molecule may bind to a polypeptide or protein molecule comprising an amino acid sequence comprising residues 337 to 368 of SEQ ID NO: 1 with a K_{D} of less than 25 nM, preferably less than 20 nM, 15 nM or 10 nM. The K_{D} may preferably be for binding to SEQ ID NO: 1 or to SEQ ID NO: 5.

The epitope of the specific binding molecule may be within an amino acid sequence comprising residues 369 to 390 of SEQ ID NO: 1. Accordingly, the epitope may be within the amino acid sequence of SEQ ID NO: 101 (KKIETHKLTFRENAKAKTDHGA).

The epitope may be within an amino acid sequence comprising residues 369 to 390 of SEQ ID NO: 1. This epitope may be bound by the CDRs of the specific binding molecule referred to as “NS4E3” herein.

The epitope may comprise the amino acid sequence of SEQ ID NO: 101 (KKIETHKLTFRENAKAKTDHGA). The epitope may comprise an amino acid sequence having at least 70%, at least 75%, at least 80%, at least 90%, at least 95% or at least 99% identity to SEQ ID NO: 101 (KKIETHKLTFRENAKAKTDHGA).

The epitope may consist of the amino acid sequence of SEQ ID NO: 101 (KKIETHKLTFRENAKAKTDHGA). The epitope may consist of an amino acid sequence having at least 70%, at least 75%, at least 80%, at least 90%, at least 95% or at least 99% identity to SEQ ID NO: 101 (KKIETHKLTFRENAKAKTDHGA).

The specific binding molecule may bind to a polypeptide or protein molecule comprising an amino acid sequence comprising residues 369 to 390 of SEQ ID NO: 1. Said specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 102 (R E S I A);
VHCDR2 comprises the sequence set forth in SEQ ID NO: 103 (G V G I D G T S Y Y S P A L K S);
VHCDR3 comprises the sequence set forth in SEQ ID NO: 104 (N Y I D F E Y);
VLCDR1 comprises the sequence set forth in SEQ ID NO: 105 (S G S S/N/Y S/N/-N/-V/-G/I Y/S/A/G E/G/S D/N/T Y/G/D V N/S/G)

VLCDR2 comprises the sequence set forth in SEQ ID NO: 106 (G/R T/N/S T/S N/T/R R P/A S); and
VLCDR3 comprises the sequence set forth in SEQ ID NO: 107 (L/A/G S Y D R/T/G/S S/T G/N S/R/-N/G/S/I F/I/V);
or for each CDR sequence, an amino acid sequence with
   (i) at least 85% identity thereto, and/or
   (ii) one, two, or three amino acid substitutions relative thereto.

Said sequence identity is at least about 85% sequence identity and may therefore be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity. Preferably said sequence identity is at least 90% or at least 95%.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 102 (RESIA);
VHCDR2 comprises the sequence set forth in SEQ ID NO: 103 (GVGIDGTSYYSPALKS);
VHCDR3 comprises the sequence set forth in SEQ ID NO: 104 (NYIDFEY);
VLCDR1 comprises the sequence set forth in SEQ ID NO: 108 (SGSSSNVGYEDYVN), SEQ ID NO: 109 (SGSNIAGNGVG), SEQ ID NO: 110 (SGSSNNVGSGDYVS), or SEQ ID NO: 111 (SGSYIGSTDVG);
VLCDR2 comprises the sequence set forth in SEQ ID NO: 112 (GTTNRPS), SEQ ID NO: 113 (GSTRRPS), SEQ ID NO: 114 (RNSNRPS), or SEQ ID NO: 115 (RTTTRAS); and
VLCDR3 comprises the sequence set forth in SEQ ID NO: 116 (LSYDRSGSNF), SEQ ID NO: 117 (ASYDTSNRGI), SEQ ID NO: 118 (GSYDGTNSF), or SEQ ID NO: 119 (ASYDSNNSIV);
or for each CDR sequence, an amino acid sequence with
   (i) at least 85% identity thereto, and/or
   (ii) one, two, or three amino acid substitutions relative thereto,
   wherein the specific binding molecule binds to a polypeptide or protein molecule comprising an amino acid sequence comprising residues 369 to 390 of SEQ ID NO: 1.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 102 (RESIA);
VHCDR2 comprises the sequence set forth in SEQ ID NO: 103 (GVGIDGTSYYSPALKS);
VHCDR3 comprises the sequence set forth in SEQ ID NO: 104 (NYIDFEY);
VLCDR1 comprises the sequence set forth in SEQ ID NO: 108 (SGSSSNVGYEDYVN);
VLCDR2 comprises the sequence set forth in SEQ ID NO: 112 (GTTNRPS); and
VLCDR3 comprises the sequence set forth in SEQ ID NO: 116 (LSYDRSGSNF);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 369 to 390 of SEQ ID NO: 1. The specific binding molecule comprising CDRs having 100% identity to those given above is referred to as “NS4E4” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 102 (RESIA);
VHCDR2 comprises the sequence set forth in SEQ ID NO: 103 (GVGIDGTSYYSPALKS);
VHCDR3 comprises the sequence set forth in SEQ ID NO: 104 (NYIDFEY);
VLCDR1 comprises the sequence set forth in SEQ ID NO: 108 (SGSSSNVGYEDYVN);
VLCDR2 comprises the sequence set forth in SEQ ID NO: 112 (GTTNRPS); and
VLCDR3 comprises the sequence set forth in SEQ ID NO: 116 (LSYDRSGSNF).

The specific binding molecule may comprise the CDR sequences of a clone set out in Table 4 below. The epitope may be within residues 369 to 390 of SEQ ID NO:1.

TABLE 4

| Clone | VH | | | VL | | | |
|---|---|---|---|---|---|---|---|
| name | VHCDR1 | VHCDR2 | VHCDR3 | VLCDR1 | VLCDR2 | VLCDR3 | Epitope |
| NS3E5 | RESIA | GVGIDGTSYYSPALKS | NYIDFEY | SGSY---IGSTD | GSTRRPS | ASYDSNN | 369-390 |
| | (SEQ ID | (SEQ ID NO: 103) | (SEQ ID | VG | (SEQ | SIV (SEQ | |
| | NO: 102) | | NO: 104) | (SEQ ID NO: 111) | ID NO: 113) | ID NO: 119) | |
| NS3H4 | RESIA | GVGIDGTSYYSPALKS | NYIDFEY | SGSN---IAGNGVG | RNSNRPS (SEQ | GSYDGTN-SF | 369-390 |
| | (SEQ ID | (SEQID NO: 103) | (SEQ ID | (SEQ ID NO: 109) | ID NO: 114) | (SEQ ID NO: | |
| | NO: 102) | | NO: 104) | | | 118) | |
| NS4F2 | RESIA | GVGIDGTSYYSPALKS | NYIDFEY | SGSSNNVGSGDYVS | RTTTRAS (SEQ | ASYDTSNRGI | 369-390 |
| | (SEQ ID | (SEQID NO: 103) | (SEQ ID | (SEQ ID NO: 110) | ID NO: 115) | (SEQ ID NO: | |
| | NO: 102) | | NO: 104) | | | 117) | |
| NS4E3 | RESIA | GVGIDGTSYYSPALKS | NYIDFEY | SGSSSNVGYEDYVN | GTTNRPS (SEQ | LSYDRSGSNF | 369-390 |
| | (SEQ ID | (SEQID NO: 103) | (SEQ ID | (SEQ ID NO: 108) | ID NO: 112) | (SEQ ID NO: | |
| | NO: 102) | | NO: 104) | | | 116) | |

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises a VHCDR1 amino acid sequence set forth in table 4;
VHCDR2 comprises a VHCDR2 amino acid sequence set forth in table 4;
VHCDR3 comprises a VHCDR3 amino acid sequence set forth in table 4;
VLCDR1 comprises a VLCDR1 amino acid sequence set forth in table 4;
VLCDR2 comprises a VLCDR2 amino acid sequence set forth in table 4; and
VLCDR3 comprises a VLCDR3 amino acid sequence set forth in table 4;
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 369 to 390 of SEQ ID NO: 1.

The specific binding molecule may bind to a polypeptide or protein molecule comprising an amino acid sequence comprising residues 369 to 390 of SEQ ID NO: 1 with a K_{D} of less than 25 nM, preferably less than 20 nM, 15 nM or 10 nM. The K_{D} may preferably be for binding to SEQ ID NO: 1 or to SEQ ID NO: 5.

The epitope of the specific binding molecule may be within an amino acid sequence comprising residues 412 to 441 of SEQ ID NO: 1. Accordingly, the epitope may be within the amino acid sequence of SEQ ID NO: 120 (SSTGSIDMVDSPQLATLADEVSASLAKQGL).

The epitope may be within an amino acid sequence comprising residues 412 to 441 of SEQ ID NO: 1. This epitope may be bound by the CDRs of the specific binding molecule referred to as “412E10” herein.

The epitope may comprise sequence of SEQ ID NO: 120 (SSTGSIDMVDSPQLATLADEVSASLAKQGL). The epitope may comprise an amino acid sequence having at least 70%, at least 75%, at least 80%, at least 90%, at least 95% or at least 99% identity to SEQ ID NO: 120 (SSTGSIDMVDSPQLATLADEVSASLAKQGL).

The epitope may consist of the amino acid sequence of SEQ ID NO: 120 (SSTGSIDMVDSPQLATLADEVSASLAKQGL). The epitope may consist of an amino acid sequence having at least 70%, at least 75%, at least 80%, at least 90%, at least 95% or at least 99% identity to SEQ ID NO: 120 (SSTGSIDMVDSPQLATLADEVSASLAKQGL).

The specific binding molecule may bind to a polypeptide or protein molecule comprising an amino acid sequence comprising residues 412 to 441 of SEQ ID NO: 1. Said specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 121 (S/N D/Y S/G/A V/L A/G);
VHCDR2 comprises the sequence set forth in SEQ ID NO: 122 (A/N S/I G/Y/W S/R S/G G N/S/R K/T/I Y/EYN PAL K S);
VHCDR3 comprises the sequence set forth in SEQ ID NO: 123 (G I/G I/V A/G G/S V D V), or SEQ ID NO: 124 (SGGD);
VLCDR1 comprises the sequence set forth in SEQ ID NO: 125 (S G S/G S/N N V/I G Y/R G N/D/T Y/F V G/D);
VLCDR2 comprises the sequence set forth in SEQ ID NO: 126 (G T/A A/D/T I/S/R R A/P S/P); and
VLCDR3 comprises the sequence set forth in SEQ ID NO: 127 (A S/T Y Q/D S/Y/R N/S Y/D/N/E A/G/D/S-/G/M/V-/I F/V/I);
or for each CDR sequence, an amino acid sequence with
   (i) at least 85% identity thereto, and/or
   (ii) one, two, or three amino acid substitutions relative thereto.

Said sequence identity is at least about 85% sequence identity and may therefore be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity. Preferably said sequence identity is at least 90% or at least 95%.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 128 (SDSVA), SEQ ID NO: 129 (NYGVG), or SEQ ID NO: 130 (SYALG);
VHCDR2 comprises the sequence set forth in SEQ ID NO: 131 (ASGSSGNKYYNPALKS), SEQ ID NO: 132 (NIWRGGRIEYNPALKS), or SEQ ID NO: 133 (NIYSGGSTYYNPALKS);
VHCDR3 comprises the sequence set forth in SEQ ID NO: 134 (GIIAGVDV), SEQ ID NO: 135 (GGVGSVDV), or SEQ ID NO: 124 (SGGD);
VLCDR1 comprises the sequence set forth in SEQ ID NO: 39 (SGSSSNVGYGNYVG), SEQ ID NO: 137 (SGGRNNIGRGTFVD), and SEQ ID NO: 138 (SGSSSNVGYGDYVG);
VLCDR2 comprises the sequence set forth in SEQ ID NO: 139 (GTAIRAS), SEQ ID NO: 140 (GAASRAS), SEQ ID NO: 141 (GATSRAS), or SEQ ID NO: 142 (GTDRRPP); and
VLCDR3 comprises the sequence set forth in SEQ ID NO: 143 (ASYQSNYAF), SEQ ID NO: 144 (ASYDRSESVV), SEQ ID NO: 145 (ASYDSSDGGV), or SEQ ID NO 146 (ATYDYSNDMII);
or for each CDR sequence, an amino acid sequence with
   (i) at least 85% identity thereto, and/or
   (ii) one, two, or three amino acid substitutions relative thereto,
   wherein the specific binding molecule binds to a polypeptide or protein molecule comprising an amino acid sequence comprising residues 412 to 441 of SEQ ID NO: 1.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 128 (SDSVA);
VHCDR2 comprises the sequence set forth in SEQ ID NO: 131 (ASGSSGNKYYNPALKS);
VHCDR3 comprises the sequence set forth in SEQ ID NO: 134 (GIIAGVDV);
VLCDR1 comprises the sequence set forth in SEQ ID NO: 39 (SGSSSNVGYGNYVG);
VLCDR2 comprises the sequence set forth in SEQ ID NO: 139 (GTAIRAS); and
VLCDR3 comprises the sequence set forth in SEQ ID NO: 143 (ASYQSNYAF);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 412 to 441 of SEQ ID NO: 1. The specific binding molecule comprising CDRs having 100% identity to those given above is referred to as “412E10” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 128 (SDSVA);
VHCDR2 comprises the sequence set forth in SEQ ID NO: 131 (ASGSSGNKYYNPALKS);
VHCDR3 comprises the sequence set forth in SEQ ID NO: 134 (GIIAGVDV);
VLCDR1 comprises the sequence set forth in SEQ ID NO: 39 (SGSSSNVGYGNYVG);
VLCDR2 comprises the sequence set forth in SEQ ID NO: 139 (GTAIRAS); and
VLCDR3 comprises the sequence set forth in SEQ ID NO: 143 (ASYQSNYAF).

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 459 (QVQLQESGPSLVKPSQTLSLTCTVSGFSVI);
VHFR2 comprises the sequence set forth in SEQ ID NO: 460 (WVRQAPGKVPEWLG);
VHFR3 comprises the sequence set forth in SEQ ID NO: 461 (RLSITRDTSKSQVSLSLSSVTTEDTAVYYCAR);
VHFR4 comprises the sequence set forth in SEQ ID NO: 462 (WGRGLLVTVSS);
VLFR1 comprises the sequence set forth in SEQ ID NO: 463 (QAVLTQPSSVSGSLGQRVSITC);
VLFR2 comprises the sequence set forth in SEQ ID NO: 464 (WYQQVPGSAPKLLIY);
VLFR3 comprises the sequence set forth in SEQ ID NO: 465 (GVPDRFSGSRSGDTATLTITSLQAEDEADYYC);
VLFR4 comprises the sequence set forth in SEQ ID NO: 466 (FGSGTRLTVLG);
or for each FR sequence, an amino acid sequence with
   (i) at least 50% identity thereto, and/or
   (ii) one, two, three, four or five amino acid substitutions relative thereto.

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 459 (QVQLQESGPSLVKPSQTLSLTCTVSGFSVI);
VHFR2 comprises the sequence set forth in SEQ ID NO: 460 (WVRQAPGKVPEWLG);
VHFR3 comprises the sequence set forth in SEQ ID NO: 461 (RLSITRDTSKSQVSLSLSSVTTEDTAVYYCAR);
VHFR4 comprises the sequence set forth in SEQ ID NO: 462 (WGRGLLVTVSS);
VLFR1 comprises the sequence set forth in SEQ ID NO: 463 (QAVLTQPSSVSGSLGQRVSITC);
VLFR2 comprises the sequence set forth in SEQ ID NO: 464 (WYQQVPGSAPKLLIY);
VLFR3 comprises the sequence set forth in SEQ ID NO: 465 (GVPDRFSGSRSGDTATLTITSLQAEDEADYYC);
VLFR4 comprises the sequence set forth in SEQ ID NO: 466 (FGSGTRLTVLG);
or for each FR sequence, an amino acid sequence with
   (i) at least 50% identity thereto, and/or
   (ii) one, two, three, four or five amino acid substitutions relative thereto,
wherein the specific binding molecule binds to a polypeptide or protein molecule comprising an amino acid sequence comprising residues 412 to 441 of SEQ ID NO: 1. A specific binding molecule comprising FRs having 100% identity to those given above is referred to as “412E10” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 459 (QVQLQESGPSLVKPSQTLSLTCTVSGFSVI);
   VHFR2 comprises the sequence set forth in SEQ ID NO: 460 (WVRQAPGKVPEWLG);
   VHFR3 comprises the sequence set forth in SEQ ID NO: 461 (RLSITRDTSKSQVSLSLSSVTTEDTAVYYCAR);
   VHFR4 comprises the sequence set forth in SEQ ID NO: 462 (WGRGLLVTVSS);
   VLFR1 comprises the sequence set forth in SEQ ID NO: 463 (QAVLTQPSSVSGSLGQRVSITC);
   VLFR2 comprises the sequence set forth in SEQ ID NO: 464 (WYQQVPGSAPKLLIY);
   VLFR3 comprises the sequence set forth in SEQ ID NO: 465 (GVPDRFSGSRSGDTATLTITSLQAEDEADYYC);
   VLFR4 comprises the sequence set forth in SEQ ID NO: 466 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 128 (SDSVA);
   VHCDR2 comprises the sequence set forth in SEQ ID NO: 131 (ASGSSGNKYYNPALKS);
   VHCDR3 comprises the sequence set forth in SEQ ID NO: 134 (GIIAGVDV);
   VLCDR1 comprises the sequence set forth in SEQ ID NO: 39 (SGSSSNVGYGNYVG);
   VLCDR2 comprises the sequence set forth in SEQ ID NO: 139 (GTAIRAS); and
   VLCDR3 comprises the sequence set forth in SEQ ID NO: 143 (ASYQSNYAF);
   or for each CDR sequence, an amino acid sequence with
      (i) at least 85% identity thereto, and/or
      (ii) one, two, or three amino acid substitutions relative thereto
   wherein the specific binding molecule binds to a polypeptide or protein molecule comprising an amino acid sequence comprising residues 412 to 441 of SEQ ID NO: 1. The specific binding molecule comprising FRs and CDRs having 100% identity to those given above is referred to as “412E10” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 459 (QVQLQESGPSLVKPSQTLSLTCTVSGFSVI);
   VHFR2 comprises the sequence set forth in SEQ ID NO: 460 (WVRQAPGKVPEWLG);
   VHFR3 comprises the sequence set forth in SEQ ID NO: 461 (RLSITRDTSKSQVSLSLSSVTTEDTAVYYCAR);
   VHFR4 comprises the sequence set forth in SEQ ID NO: 462 (WGRGLLVTVSS);
   VLFR1 comprises the sequence set forth in SEQ ID NO: 463 (QAVLTQPSSVSGSLGQRVSITC);
   VLFR2 comprises the sequence set forth in SEQ ID NO: 464 (WYQQVPGSAPKLLIY);
   VLFR3 comprises the sequence set forth in SEQ ID NO: 465 (GVPDRFSGSRSGDTATLTITSLQAEDEADYYC);
   VLFR4 comprises the sequence set forth in SEQ ID NO: 466 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 128 (SDSVA);
   VHCDR2 comprises the sequence set forth in SEQ ID NO: 131 (ASGSSGNKYYNPALKS);
   VHCDR3 comprises the sequence set forth in SEQ ID NO: 134 (GIIAGVDV);
   VLCDR1 comprises the sequence set forth in SEQ ID NO: 39 (SGSSSNVGYGNYVG);
   VLCDR2 comprises the sequence set forth in SEQ ID NO: 139 (GTAIRAS); and
   VLCDR3 comprises the sequence set forth in SEQ ID NO: 143 (ASYQSNYAF);
   wherein the specific binding molecule binds to a polypeptide or protein molecule comprising an amino acid sequence comprising residues 412 to 441 of SEQ ID NO: 1. The specific binding molecule comprising FRs and CDRs having 100% identity to those given above is referred to as “412E10” herein.

The specific binding molecule may comprise:
(a) A VH domain comprising the sequence set forth in SEQ ID NO: 467 (QVQLQESGPSLVKPSQTLSLTCTVSGFSVISDSVAWVRQAPGKVPEWLGASGSSGNKY YNPALKSRLSITRDTSKSQVSLSLSSVTTEDTAVYYCARGIIAGVDVWGRGLLVTVSS); and/or
(b) a VL domain comprising the sequence set forth in SEQ ID NO: 468 (QAVLTQPSSVSGSLGQRVSITCSGSSSNVGYGNYVGWYQQVPGSAPKLLIYGTAIRASG VPDRFSGSRSGDTATLTITSLQAEDEADYYCASYQSNYAFFGSGTRLTVLG);
or a humanized variant thereof.

The specific binding molecule may comprise:
(a) A heavy chain comprising the sequence set forth in SEQ ID NO: 469 (QVQLQESGPSLVKPSQTLSLTCTVSGFSVISDSVAWVRQAPGKVPEWLGASGSSGNKY YNPALKSRLSITRDTSKSQVSLSLSSVTTEDTAVYYCARGIIAGVDVWGRGLLVTVSSAKT TAPSVYPLAPVCGDTTGSSVTLGCLVKGYFPEPVTLTWNSGSLSSGVHTFPAVLQSDLYT LSSSVTVTSSTWPSQSITCNVAHPASSTKVDKKIEPRGPTIKPCPPCKCPAPNLLGGPSVFI FPPKIKDVLMISLSPIVTCVVVDVSEDDPDVQISWFVNNVEVHTAQTQTHREDYNSTLRVV SALPIQHQDWMSGKEFKCKVNNKDLPAPIERTISKPKGSVRAPQVYVLPPPEEEMTKKQV TLTCMVTDFMPEDIYVEWTNNGKTELNYKNTEPVLDSDGSYFMYSKLRVEKKNWVERNS YSCSVVHEGLHNHHTTKSFSRTPGK); and/or
(b) a light chain comprising the sequence set forth in SEQ ID NO: 470 (QAVLTQPSSVSGSLGQRVSITCSGSSSNVGYGNYVGWYQQVPGSAPKLLIYGTAIRASG VPDRFSGSRSGDTATLTITSLQAEDEADYYCASYQSNYAFFGSGTRLTVLGGQPKSSPSV TLFPPSSEELETNKATLVCTITDFYPGVVTVDWKVDGTPVTQGMETTQPSKQSNNKYMAS SYLTLTARAWERHSSYSCQVTHEGHTVEKSLSRADCS);
or a humanized variant thereof.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 128 (SDSVA);
VHCDR2 comprises the sequence set forth in SEQ ID NO: 131 (ASGSSGNKYYNPALKS);
VHCDR3 comprises the sequence set forth in SEQ ID NO: 134 (GIIAGVDV);
VLCDR1 comprises the sequence set forth in SEQ ID NO: 138 (SGSSSNVGYGDYVG);
VLCDR2 comprises the sequence set forth in SEQ ID NO: 140 (GAASRAS); and
VLCDR3 comprises the sequence set forth in SEQ ID NO: 145 (ASYDSSDGGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 412 to 441 of SEQ ID NO: 1. The specific binding molecule comprising CDRs having 100% identity to those given above is referred to as “412B9” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 128 (SDSVA);
VHCDR2 comprises the sequence set forth in SEQ ID NO: 131 (ASGSSGNKYYNPALKS);
VHCDR3 comprises the sequence set forth in SEQ ID NO: 134 (GIIAGVDV);
VLCDR1 comprises the sequence set forth in SEQ ID NO: 138 (SGSSSNVGYGDYVG);
VLCDR2 comprises the sequence set forth in SEQ ID NO: 140 (GAASRAS); and
VLCDR3 comprises the sequence set forth in SEQ ID NO: 145 (ASYDSSDGGV).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 129 (NYGVG);
VHCDR2 comprises the sequence set forth in SEQ ID NO: 133 (NIYSGGSTYYNPALKS);
VHCDR3 comprises the sequence set forth in SEQ ID NO: 135 (GGVGSVDV);
VLCDR1 comprises the sequence set forth in SEQ ID NO: 137 (SGGRNNIGRGTFVD);
VLCDR2 comprises the sequence set forth in SEQ ID NO: 142 (GTDRRPP); and
VLCDR3 comprises the sequence set forth in SEQ ID NO: 146 (ATYDYSNDMII);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 412 to 441 of SEQ ID NO: 1. The specific binding molecule comprising CDRs having 100% identity to those given above is referred to as “412E6” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 129 (NYGVG);
VHCDR2 comprises the sequence set forth in SEQ ID NO: 133 (NIYSGGSTYYNPALKS);
VHCDR3 comprises the sequence set forth in SEQ ID NO: 135 (GGVGSVDV);
VLCDR1 comprises the sequence set forth in SEQ ID NO: 137 (SGGRNNIGRGTFVD);
VLCDR2 comprises the sequence set forth in SEQ ID NO: 142 (GTDRRPP); and
VLCDR3 comprises the sequence set forth in SEQ ID NO: 146 (ATYDYSNDMII).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 130 (SYALG);
VHCDR2 comprises the sequence set forth in SEQ ID NO: 132 (NIWRGGRIEYNPALKS);
VHCDR3 comprises the sequence set forth in SEQ ID NO: 124 (SGGD);
VLCDR1 comprises the sequence set forth in SEQ ID NO: 39 (SGSSSNVGYGNYVG);
VLCDR2 comprises the sequence set forth in SEQ ID NO: 141 (GATSRAS); and
VLCDR3 comprises the sequence set forth in SEQ ID NO: 144 (ASYDRSESVV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 412 to 441 of SEQ ID NO: 1. The specific binding molecule comprising CDRs having 100% identity to those given above is referred to as “412G11” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 130 (SYALG);
VHCDR2 comprises the sequence set forth in SEQ ID NO: 132 (NIWRGGRIEYNPALKS);
VHCDR3 comprises the sequence set forth in SEQ ID NO: 124 (SGGD);
VLCDR1 comprises the sequence set forth in SEQ ID NO: 39 (SGSSSNVGYGNYVG);
VLCDR2 comprises the sequence set forth in SEQ ID NO: 141 (GATSRAS); and
VLCDR3 comprises the sequence set forth in SEQ ID NO: 144 (ASYDRSESVV).

The specific binding molecule may comprise the CDR sequences of a clone set out in Table 5 below. The epitope may be within residues 412 to 441 of SEQ ID NO: 1.

TABLE 5

| Clone | VH | | | VL | | | |
|---|---|---|---|---|---|---|---|
| name | CDR1 | CDR2 | CDR3 | CDR1 | CDR2 | CDR3 | Epitope |
| 412E10 | SDSVA | ASGSSGNKYYNPAL | GIIAGVDV | SGSSSNVGYGNYVG | GTAIRAS | ASYQSNYAF | 412-441 |
| | (SEQ ID | KS | (SEQ ID | (SEQ ID NO: 39) | (SEQ ID | (SEQ ID | |
| | NO: 128) | (SEQ ID NO: 131) | NO: 134) | | NO: 139) | NO: 143) | |
| 412B9 | SDSVA | ASGSSGNKYYNPAL | GIIAGVDV | SGSSSNVGYGDYVG | GAASRAS | ASYDSSDGGV | 412-441 |
| | (SEQ ID | KS | (SEQ ID | (SEQ ID NO: 138) | (SEQ ID | (SEQ ID | |
| | NO: 128) | (SEQ ID NO: 131) | NO: 134) | | NO: 140) | NO: 145) | |
| 412E6 | NYGVG | NIYSGGSTYYNPALK | GGVGSVDV | SGGRNNIGRGTFVD | GTDRRPP | ATYDYSNDMII | 412-441 |
| | (SEQ ID | S | (SEQ ID | (SEQ ID NO: 137) | (SEQ ID | (SEQ ID | |
| | NO: 129) | (SEQ ID NO: 133) | NO: 135) | | NO: 142) | NO: 146) | |
| 412G11 | SYALG | NIWRGGRIEYNPALK | SGGD | SGSSSNVGYGNYVG | GATSRAS | ASYDRSESVV | 412-441 |
| | (SEQ ID | S | (SEQ ID | (SEQ ID NO: 39) | (SEQ ID | (SEQ ID | |
| | NO: 130) | (SEQ ID NO: 132) | NO: 124) | | NO: 141) | NO: 144) | |

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises a VHCDR1 amino acid sequence set forth in table 5;
VHCDR2 comprises a VHCDR2 amino acid sequence set forth in table 5;
VHCDR3 comprises a VHCDR3 amino acid sequence set forth in table 5;
VLCDR1 comprises a VLCDR1 amino acid sequence set forth in table 5;
VLCDR2 comprises a VLCDR2 amino acid sequence set forth in table 5; and
VLCDR3 comprises a VLCDR3 amino acid sequence set forth in table 5;
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 412 to 441 of SEQ ID NO: 1.

The specific binding molecule may bind to a polypeptide or protein molecule comprising an amino acid sequence comprising residues 412 to 441 of SEQ ID NO: 1 with a K_{D} of less than around 25 nM. The K_{D} may be less than around 20 nM, less than around 15 nM, or less than around 10 nM. The K_{D} may preferably be for binding to SEQ ID NO: 1 or to SEQ ID NO: 120. The K_{D} for binding to SEQ ID NO: 1 may be around 1 nM to around 10 nM. The K_{D} for binding to SEQ ID NO: 1 may be around 3.16 nM or 9.0 nM, optionally wherein the specific binding molecule comprises the CDRs of 412E10.

The specific binding molecule may comprise an amino acid sequence having at least 70%, at least 75%, at least 80%, at least 90%, at least 95% or at least 99% identity to SEQ ID NO: 147 wherein the specific binding molecule binds to a polypeptide or protein molecule comprising an amino acid sequence comprising residues 412 to 441 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 147. The CDRs may be 100% identical to the CDRs of SEQ ID NO: 147. The specific binding molecule may comprise an amino acid sequence having at least 70%, at least 75%, at least 80%, at least 90%, at least 95% or at least 99% identity to SEQ ID NO: 147 wherein CDRs are 100% identical to the CDRs of SEQ ID NO: 147. The specific binding molecule may comprise the amino acid sequence of SEQ ID NO: 147.

(412E10 amino acid sequence)
SEQ ID NO: 147
QVQLQESGPSLVKPSQTLSLTCTVSGFSVISDSVAWVRQAPGKVPEWLG
ASGSSGNKYYNPALKSRLSITRDTSKSQVSLSLSSVTTEDTAVYYCARG
IIAGVDVWGRGLLVTVSSEGKSSGASGESKVDDQAVLTQPSSVSGSLGQ
RVSITCSGSSSNVGYGNYVGWYQQVPGSAPKLLIYGTAIRASGVPDRFS
GSRSGDTATLTITSLQAEDEADYYCASYQSNYAFFGSGTRLTVLG

The specific binding molecule may comprise an amino acid sequence having at least 70%, at least 75%, at least 80%, at least 90%, at least 95% or at least 99% identity to SEQ ID NO: 417 wherein the specific binding molecule binds to a polypeptide or protein molecule comprising an amino acid sequence comprising residues 412 to 441 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 417. The CDRs may be 100% identical to the CDRs of SEQ ID NO: 417. The specific binding molecule may comprise an amino acid sequence having at least 70%, at least 75%, at least 80%, at least 90%, at least 95% or at least 99% identity to SEQ ID NO: 417 wherein CDRs are 100% identical to the CDRs of SEQ ID NO: 417. The specific binding molecule may comprise the amino acid sequence of SEQ ID NO: 417.

(412B9 amino acid sequence)
SEQ ID NO: 417
QVQLQESGPSLVKPSQTLSLTCTVSGFSVISDSVAWVRQAPGKVPEWLG
ASGSSGNKYYNPALKSRLSITRDTSKSQVSLSLSSVTTEDTAVYYCARG
IIAGVDVWGRGLLVSVSSEGKSSGASGESKVDDQAVLTQPSSVSGALGQ
RVSITCSGSSSNVGYGDYVGWYQQVPGSAPKLLIYGAASRASGVPDRFS
GSRSGNTATLTISSLQAEDEADYYCASYDSSDGGVFGSGTRLTVLG

The specific binding molecule may comprise an amino acid sequence having at least 70%, at least 75%, at least 80%, at least 90%, at least 95% or at least 99% identity to SEQ ID NO: 418 wherein the specific binding molecule binds to a polypeptide or protein molecule comprising an amino acid sequence comprising residues 412 to 441 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 418. The CDRs may be 100% identical to the CDRs of SEQ ID NO: 418. The specific binding molecule may comprise an amino acid sequence having at least 70%, at least 75%, at least 80%, at least 90%, at least 95% or at least 99% identity to SEQ ID NO: 418 wherein CDRs are 100% identical to the CDRs of SEQ ID NO: 418. The specific binding molecule may comprise the amino acid sequence of SEQ ID NO: 418.

(412E6 amino acid sequence)
SEQ ID NO: 418
QVQLQESGPSLVKPSETLSLTCTVSGFSLTNYGVGWVRQAPGKALEWLG
NIYSGGSTYYNPALKSRLSITRDTSKSQVSLSLNSVTLEDTAVYYCGRG
GVGSVDVWGPGLLVTVSSEGKSSGASGESKVDDQAVLTQPPSVSGSPGQ
RVSITCSGGRNNIGRGTFVDWYQQLPGSGLKTVIYGTDRRPPGVPDRFS
GSKTGNAATLTITSLQAEDEADYWCATYDYSNDMIILGSGTRLTVLG

The specific binding molecule may comprise an amino acid sequence having at least 70%, at least 75%, at least 80%, at least 90%, at least 95% or at least 99% identity to SEQ ID NO: 434 wherein the specific binding molecule binds to a polypeptide or protein molecule comprising an amino acid sequence comprising residues 412 to 441 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 434. The CDRs may be 100% identical to the CDRs of SEQ ID NO: 434. The specific binding molecule may comprise an amino acid sequence having at least 70%, at least 75%, at least 80%, at least 90%, at least 95% or at least 99% identity to SEQ ID NO: 434 wherein CDRs are 100% identical to the CDRs of SEQ ID NO: 434. The specific binding molecule may comprise the amino acid sequence of SEQ ID NO: 434.

(412G11 amino acid sequence)
SEQ ID NO: 434
QVRLQESGPSLVKPSQTLSLTCTVSGFSLTSYALGWVRQAPGRAPEWIG
NIWRGGRIEYNPALKSRLSITRDTSKSQVSLSLSSVTTEDTAVYYCSRS
GGDWGPGLLVTVSSEGKSSGASGESKVDDQAVLTQPSSVSGSLGQRVSI
TCSGSSSNVGYGNYVGWYQQVPGSAPKLLIYGATSRASGVPDRFSGSRS
ENTATLTISSLQAEDEADYYCASYDRSESVVFGSGTRLTVLG

The epitope of the specific binding molecule may be within an amino acid sequence comprising residues 1 to 49 of SEQ ID NO: 1. Accordingly, the epitope may be within the amino acid sequence of SEQ ID NO: 148 (MAEPRQEFEVMEDHAGTYGLGDRKDQGGYTMHQDQEGDTDAGLKESPLQ). Preferably, the epitope of the specific binding molecule within an amino acid sequence comprising residues 1 to 49 of SEQ ID NO: 1, may be within an amino acid sequence comprising residues 1 to 15 of SEQ ID NO: 1.

The epitope may be within an amino acid sequence comprising residues 1 to 49 of SEQ ID NO: 1. This epitope may be bound by the CDRs of the specific binding molecule referred to as “3aG3” herein.

The epitope may be within an amino acid sequence comprising residues 1 to 49 of SEQ ID NO: 1, preferably within an amino acid sequence comprising residues 1 to 15 of SEQ ID NO: 1. This epitope may be bound by the CDRs of the specific binding molecule referred to as “3bG4” herein.

The epitope may comprise the amino acid sequence of SEQ ID NO: 148 (MAEPRQEFEVMEDHAGTYGLGDRKDQGGYTMHQDQEGDTDAGLKESPLQ). The epitope may comprise an amino acid sequence having at least 70%, at least 75%, at least 80%, at least 90%, at least 95% or at least 99% identity to SEQ ID NO: 148 (MAEPRQEFEVMEDHAGTYGLGDRKDQGGYTMHQDQEGDTDAGLKESPLQ).

The epitope may consist of the amino acid sequence of SEQ ID NO: 148 (MAEPRQEFEVMEDHAGTYGLGDRKDQGGYTMHQDQEGDTDAGLKESPLQ). The epitope may consist of an amino acid sequence having at least 70%, at least 75%, at least 80%, at least 90%, at least 95% or at least 99% identity to SEQ ID NO: 148 (MAEPRQEFEVMEDHAGTYGLGDRKDQGGYTMHQDQEGDTDAGLKESPLQ).

The specific binding molecule may bind to a polypeptide or protein molecule comprising an amino acid sequence comprising residues 1 to 49 of SEQ ID NO: 1. Said specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (S N G V G);
VHCDR2 comprises the sequence set forth in SEQ ID NO: 150 (D I S/A S S/V/G G K A/K/V Y A/S/G N/H P A L K S);
VHCDR3 comprises the sequence set forth in SEQ ID NO: 151 (C R D G G V S/T Y G Y D I/S D Y);
VLCDR1 comprises the sequence set forth in SEQ ID NO: 152 (S G S S/T S/G N I/V G G/S/Y G N/D Y/D L/V S/G);
VLCDR2 comprises the sequence set forth in SEQ ID NO: 153 (G A/V T S/N/E R/L A S); and
VLCDR3 comprises the sequence set forth in SEQ ID NO: 154 (A/G S F/Y D T/S/D S/N S G G I/V);
or for each CDR sequence, an amino acid sequence with
   (i) at least 85% identity thereto, and/or
   (ii) one, two, or three amino acid substitutions relative thereto.

Said sequence identity is at least about 85% sequence identity and may therefore be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity. Preferably said sequence identity is at least 90% or at least 95%.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (SNGVG);
VHCDR2 comprises the sequence set forth in SEQ ID NO: 155 (DISSSGKAYANPALKS), SEQ ID NO: 156 (DISSGGKVYGHPALKS), SEQ ID NO: 157 (DISSVGKKYANPALKS), or SEQ ID NO: 158 (DIASSGKAYSNPALKS);
VHCDR3 comprises the sequence set forth in SEQ ID NO: 159 (CRDGGVSYGYDIDY), SEQ ID NO: 160 (CRDGGVSYGYDSDY), or SEQ ID NO: 161 (CRDGGVTYGYDIDY);
VLCDR1 comprises the sequence set forth in SEQ ID NO: 163 (SGSSSNIGGGNYLS), SEQ ID NO: 138 (SGSSSNVGYGDYVG), SEQ ID NO: 165 (SGSSGNVGYGDYVS), or SEQ ID NO: 166 (SGSTSNVGSGNDVS);
VLCDR2 comprises the sequence set forth in SEQ ID NO: 141 (GATSRAS), SEQ ID NO: 168 (GVTERAS), SEQ ID NO: 169 (GATNLAS), or SEQ ID NO: 170 (GATNRAS); and
VLCDR3 comprises the sequence set forth in SEQ ID NO: 171 (ASFDTSSGGI), SEQ ID NO: 172 (ASYDDSSGGI), SEQ ID NO: 173 (ASYDSSSGGV), or SEQ ID NO: 174 (GSYDSNSGGI); or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 1 to 49 of SEQ ID NO: 1.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (SNGVG);
VHCDR2 comprises the sequence set forth in SEQ ID NO: 155 (DISSSGKAYANPALKS);
VHCDR3 comprises the sequence set forth in SEQ ID NO: 159 (CRDGGVSYGYDIDY);
VLCDR1 comprises the sequence set forth in SEQ ID NO: 163 (SGSSSNIGGGNYLS);
VLCDR2 comprises the sequence set forth in SEQ ID NO: 141 (GATSRAS); and
VLCDR3 comprises the sequence set forth in SEQ ID NO: 171 (ASFDTSSGGI);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 1 to 49 of SEQ ID NO: 1. The specific binding molecule comprising CDRs having 100% identity to those given above is referred to as “3aG3” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (SNGVG);
VHCDR2 comprises the sequence set forth in SEQ ID NO: 155 (DISSSGKAYANPALKS);
VHCDR3 comprises the sequence set forth in SEQ ID NO: 159 (CRDGGVSYGYDIDY);
VLCDR1 comprises the sequence set forth in SEQ ID NO: 163 (SGSSSNIGGGNYLS);
VLCDR2 comprises the sequence set forth in SEQ ID NO: 141 (GATSRAS); and
VLCDR3 comprises the sequence set forth in SEQ ID NO: 171 (ASFDTSSGGI).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (SNGVG);
VHCDR2 comprises the sequence set forth in SEQ ID NO: 156 (DISSGGKVYGHPALKS);
VHCDR3 comprises the sequence set forth in SEQ ID NO: 160 (CRDGGVSYGYDSDY);
VLCDR1 comprises the sequence set forth in SEQ ID NO: 138 (SGSSSNVGYGDYVG);
VLCDR2 comprises the sequence set forth in SEQ ID NO: 168 (GVTERAS); and
VLCDR3 comprises the sequence set forth in SEQ ID NO: 172 (ASYDDSSGGI);
or for each CDR sequence, an amino acid sequence with
(i) at least 85% identity thereto, and/or
(ii) one, two, or three amino acid substitutions relative thereto,
wherein the specific binding molecule binds to a polypeptide or protein molecule comprising an amino acid sequence comprising residues 1 to 49 of SEQ ID NO: 1. The specific binding molecule comprising CDRs having 100% identity to those given above is referred to as “3aD3” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (SNGVG);
VHCDR2 comprises the sequence set forth in SEQ ID NO: 156 (DISSGGKVYGHPALKS);
VHCDR3 comprises the sequence set forth in SEQ ID NO: 160 (CRDGGVSYGYDSDY);
VLCDR1 comprises the sequence set forth in SEQ ID NO: 138 (SGSSSNVGYGDYVG);
VLCDR2 comprises the sequence set forth in SEQ ID NO: 168 (GVTERAS); and
VLCDR3 comprises the sequence set forth in SEQ ID NO: 172 (ASYDDSSGGI).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (SNGVG);
VHCDR2 comprises the sequence set forth in SEQ ID NO: 157 (DISSVGKKYANPALKS);
VHCDR3 comprises the sequence set forth in SEQ ID NO: 161 (CRDGGVTYGYDIDY);
VLCDR1 comprises the sequence set forth in SEQ ID NO: 165 (SGSSGNVGYGDYVS);
VLCDR2 comprises the sequence set forth in SEQ ID NO: 169 (GATNLAS); and
VLCDR3 comprises the sequence set forth in SEQ ID NO: 173 (ASYDSSSGGV);
or for each CDR sequence, an amino acid sequence with
(i) at least 85% identity thereto, and/or
(ii) one, two, or three amino acid substitutions relative thereto,
wherein the specific binding molecule binds to a polypeptide or protein molecule comprising an amino acid sequence comprising residues 1 to 49 of SEQ ID NO: 1. The specific binding molecule comprising CDRs having 100% identity to those given above is referred to as “3aH6” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (SNGVG);
VHCDR2 comprises the sequence set forth in SEQ ID NO: 157 (DISSVGKKYANPALKS);
VHCDR3 comprises the sequence set forth in SEQ ID NO: 161 (CRDGGVTYGYDIDY);
VLCDR1 comprises the sequence set forth in SEQ ID NO: 165 (SGSSGNVGYGDYVS);
VLCDR2 comprises the sequence set forth in SEQ ID NO: 169 (GATNLAS); and
VLCDR3 comprises the sequence set forth in SEQ ID NO: 173 (ASYDSSSGGV).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (SNGVG);
VHCDR2 comprises the sequence set forth in SEQ ID NO: 158 (DIASSGKAYSNPALKS);
VHCDR3 comprises the sequence set forth in SEQ ID NO: 161 (CRDGGVTYGYDIDY);
VLCDR1 comprises the sequence set forth in SEQ ID NO: 166 (SGSTSNVGSGNDVS);
VLCDR2 comprises the sequence set forth in SEQ ID NO: 170 (GATNRAS); and
VLCDR3 comprises the sequence set forth in SEQ ID NO: 174 (GSYDSNSGGI);
or for each CDR sequence, an amino acid sequence with
(i) at least 85% identity thereto, and/or
(ii) one, two, or three amino acid substitutions relative thereto,
wherein the specific binding molecule binds to a polypeptide or protein molecule comprising an amino acid sequence comprising residues 1 to 49 of SEQ ID NO: 1. The specific binding molecule comprising CDRs having 100% identity to those given above is referred to as “3bG4” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (SNGVG);
VHCDR2 comprises the sequence set forth in SEQ ID NO: 158 (DIASSGKAYSNPALKS);
VHCDR3 comprises the sequence set forth in SEQ ID NO: 161 (CRDGGVTYGYDIDY);
VLCDR1 comprises the sequence set forth in SEQ ID NO: 166 (SGSTSNVGSGNDVS);
VLCDR2 comprises the sequence set forth in SEQ ID NO: 170 (GATNRAS); and
VLCDR3 comprises the sequence set forth in SEQ ID NO: 174 (GSYDSNSGGI).

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 471 (QVQLQESGPSLVKPSQTLSLTCTISGFSLI);
VHFR2 comprises the sequence set forth in SEQ ID NO: 472 (WVRQAPGKVPEWVG);
VHFR3 comprises the sequence set forth in SEQ ID NO: 473 (RLSITRDTSKSQVSLSLRSVTTEDTAVYYCVR);
VHFR4 comprises the sequence set forth in SEQ ID NO: 474 (WGPGLLVTVSS);
VLFR1 comprises the sequence set forth in SEQ ID NO: 475 (QAVLTQPSSVSKSLGQSVSITC);
VLFR2 comprises the sequence set forth in SEQ ID NO: 476 (WFQQVPGSAPKLLFY);
VLFR3 comprises the sequence set forth in SEQ ID NO: 477 (GVPDRFSGSRSGNTATLTITSLQAEDEADYYC);
VLFR4 comprises the sequence set forth in SEQ ID NO: 478 (FGSGTRLTVLG);
or for each FR sequence, an amino acid sequence with
   (i) at least 50% identity thereto, and/or
   (ii) one, two, three, four or five amino acid substitutions relative thereto.

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 471 (QVQLQESGPSLVKPSQTLSLTCTISGFSLI);
VHFR2 comprises the sequence set forth in SEQ ID NO: 472 (WVRQAPGKVPEWVG);
VHFR3 comprises the sequence set forth in SEQ ID NO: 473 (RLSITRDTSKSQVSLSLRSVTTEDTAVYYCVR);
VHFR4 comprises the sequence set forth in SEQ ID NO: 474 (WGPGLLVTVSS);
VLFR1 comprises the sequence set forth in SEQ ID NO: 475 (QAVLTQPSSVSKSLGQSVSITC);
VLFR2 comprises the sequence set forth in SEQ ID NO: 476 (WFQQVPGSAPKLLFY);
VLFR3 comprises the sequence set forth in SEQ ID NO: 477 (GVPDRFSGSRSGNTATLTITSLQAEDEADYYC);
VLFR4 comprises the sequence set forth in SEQ ID NO: 478 (FGSGTRLTVLG);
or for each FR sequence, an amino acid sequence with
   (i) at least 50% identity thereto, and/or
   (ii) one, two, three, four or five amino acid substitutions relative thereto,
wherein the specific binding molecule binds to a polypeptide or protein molecule comprising an amino acid sequence comprising residues 1 to 49 of SEQ ID NO: 1. A specific binding molecule comprising FRs having 100% identity to those given above is referred to as “3bG4” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 471 (QVQLQESGPSLVKPSQTLSLTCTISGFSLI);
   VHFR2 comprises the sequence set forth in SEQ ID NO: 472 (WVRQAPGKVPEWVG);
   VHFR3 comprises the sequence set forth in SEQ ID NO: 473 (RLSITRDTSKSQVSLSLRSVTTEDTAVYYCVR);
   VHFR4 comprises the sequence set forth in SEQ ID NO: 474 (WGPGLLVTVSS);
   VLFR1 comprises the sequence set forth in SEQ ID NO: 475 (QAVLTQPSSVSKSLGQSVSITC);
   VLFR2 comprises the sequence set forth in SEQ ID NO: 476 (WFQQVPGSAPKLLFY);
   VLFR3 comprises the sequence set forth in SEQ ID NO: 477 (GVPDRFSGSRSGNTATLTITSLQAEDEADYYC);
   VLFR4 comprises the sequence set forth in SEQ ID NO: 478 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 149 (SNGVG);
   VHCDR2 comprises the sequence set forth in SEQ ID NO: 158 (DIASSGKAYSNPALKS);
   VHCDR3 comprises the sequence set forth in SEQ ID NO: 161 (CRDGGVTYGYDIDY);
   VLCDR1 comprises the sequence set forth in SEQ ID NO: 166 (SGSTSNVGSGNDVS);
   VLCDR2 comprises the sequence set forth in SEQ ID NO: 170 (GATNRAS); and
   VLCDR3 comprises the sequence set forth in SEQ ID NO: 174 (GSYDSNSGGI);
   or for each CDR sequence, an amino acid sequence with
      (i) at least 85% identity thereto, and/or
      (ii) one, two, or three amino acid substitutions relative thereto
   wherein the specific binding molecule binds to a polypeptide or protein molecule comprising an amino acid sequence comprising residues 1 to 49 of SEQ ID NO: 1. The specific binding molecule comprising FRs and CDRs having 100% identity to those given above is referred to as “3bG4” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 471 (QVQLQESGPSLVKPSQTLSLTCTISGFSLI);
   VHFR2 comprises the sequence set forth in SEQ ID NO: 472 (WVRQAPGKVPEWVG);
   VHFR3 comprises the sequence set forth in SEQ ID NO: 473 (RLSITRDTSKSQVSLSLRSVTTEDTAVYYCVR);
   VHFR4 comprises the sequence set forth in SEQ ID NO: 474 (WGPGLLVTVSS);
   VLFR1 comprises the sequence set forth in SEQ ID NO: 475 (QAVLTQPSSVSKSLGQSVSITC);
   VLFR2 comprises the sequence set forth in SEQ ID NO: 476 (WFQQVPGSAPKLLFY);
   VLFR3 comprises the sequence set forth in SEQ ID NO: 477 (GVPDRFSGSRSGNTATLTITSLQAEDEADYYC);
   VLFR4 comprises the sequence set forth in SEQ ID NO: 478 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 149 (SNGVG);
   VHCDR2 comprises the sequence set forth in SEQ ID NO: 158 (DIASSGKAYSNPALKS);
   VHCDR3 comprises the sequence set forth in SEQ ID NO: 161 (CRDGGVTYGYDIDY);
   VLCDR1 comprises the sequence set forth in SEQ ID NO: 166 (SGSTSNVGSGNDVS);
   VLCDR2 comprises the sequence set forth in SEQ ID NO: 170 (GATNRAS); and
   VLCDR3 comprises the sequence set forth in SEQ ID NO: 174 (GSYDSNSGGI);
   wherein the specific binding molecule binds to a polypeptide or protein molecule comprising an amino acid sequence comprising residues 1 to 49 of SEQ ID NO: 1. The specific binding molecule comprising FRs and CDRs having 100% identity to those given above is referred to as “3bG4” herein.

The specific binding molecule may comprise:
(a) A VH domain comprising the sequence set forth in SEQ ID NO: 479 (QVQLQESGPSLVKPSQTLSLTCTISGFSLISNGVGWVRQAPGKVPEWVGDIASSGKAYS NPALKSRLSITRDTSKSQVSLSLRSVTTEDTAVYYCVRCRDGGVTYGYDIDYWGPGLLVT VSS); and/or
(b) a VL domain comprising the sequence set forth in SEQ ID NO: 480 (QAVLTQPSSVSKSLGQSVSITCSGSTSNVGSGNDVSWFQQVPGSAPKLLFYGATNRAS GVPDRFSGSRSGNTATLTITSLQAEDEADYYCGSYDSNSGGIFGSGTRLTVLG);
or a humanized variant thereof.

The specific binding molecule may comprise:
(a) A heavy chain comprising the sequence set forth in SEQ ID NO: 481 (QVQLQESGPSLVKPSQTLSLTCTISGFSLISNGVGWVRQAPGKVPEWVGDIASSGKAYS NPALKSRLSITRDTSKSQVSLSLRSVTTEDTAVYYCVRCRDGGVTYGYDIDYWGPGLLVT VSSAKTTAPSVYPLAPVCGDTTGSSVTLGCLVKGYFPEPVTLTWNSGSLSSGVHTFPAVL QSDLYTLSSSVTVTSSTWPSQSITCNVAHPASSTKVDKKIEPRGPTIKPCPPCKCPAPNLL GGPSVFIFPPKIKDVLMISLSPIVTCVVVDVSEDDPDVQISWFVNNVEVHTAQTQTHREDY NSTLRVVSALPIQHQDWMSGKEFKCKVNNKDLPAPIERTISKPKGSVRAPQVYVLPPPEE EMTKKQVTLTCMVTDFMPEDIYVEWTNNGKTELNYKNTEPVLDSDGSYFMYSKLRVEKK NWVERNSYSCSVVHEGLHNHHTTKSFSRTPGK); and/or
(b) a light chain comprising the sequence set forth in SEQ ID NO: 482 (QAVLTQPSSVSKSLGQSVSITCSGSTSNVGSGNDVSWFQQVPGSAPKLLFYGATNRAS GVPDRFSGSRSGNTATLTITSLQAEDEADYYCGSYDSNSGGIFGSGTRLTVLGGQPKSSP SVTLFPPSSEELETNKATLVCTITDFYPGVVTVDWKVDGTPVTQGMETTQPSKQSNNKYM ASSYLTLTARAWERHSSYSCQVTHEGHTVEKSLSRADCS);
or a humanized variant thereof.

The specific binding molecule may comprise the CDR sequences of a clone set out in Table 6 below. The epitope may be within residues 1 to 49 of SEQ ID NO: 1.

TABLE 6

| Clone | VH | | | VL | | | |
|---|---|---|---|---|---|---|---|
| name | CDR1 | CDR2 | CDR3 | CDR1 | CDR2 | CDR3 | Epitope |
| 3aD3 | SNGVG | DISSGGKVYGHP | CRDGGVSYGYDSDY | SGSSSNVGYGDYV | GVTERAS | ASYDDSSGGI | 1-49 |
| | (SEQ ID | ALKS (SEQ | (SEQ ID) | G (SEQ ID | (SEQ ID | (SEQ | |
| | NO: 149) | ID NO: 156) | NO: 160 | NO: 138) | NO: 168) | ID NO: 172) | |
| 3aH6 | SNGVG | DISSVGKKYANP | CRDGGVTYGYDIDY | SGSSGNVGYGDYV | GATNLAS | ASYDSSSGGV | 1-49 |
| | (SEQ ID | ALKS (SEQ | (SEQ ID | S (SEQ ID | (SEQ ID | (SEQ | |
| | NO: 149) | ID NO: 157) | NO: 161) | NO: 165) | NO: 169) | ID NO: 173) | |
| 3aG3 | SNGVG | DISSSGKAYANP | CRDGGVSYGYDIDY | SGSSSNIGGGNYLS | GATSRAS | ASFDTSSGGI | 1-49 |
| | (SEQ ID | ALKS (SEQ | (SEQ ID | (SEQ ID | (SEQ ID | (SEQ | |
| | NO: 149) | ID NO: 155) | NO: 159) | NO: 163) | NO: 141) | ID NO: 171) | |
| 3bG4 | SNGVG | DIASSGKAYSNP | CRDGGVTYGYDIDY | SGSTSNVGSGNDV | GATNRAS | GSYDSNSGGI | 1-49 |
| | (SEQ ID | ALKS (SEQ | (SEQ ID | S (SEQ ID | (SEQ ID | (SEQ | |
| | NO: 149) | ID NO: 158) | NO: 161) | NO: 166) | NO: 170) | ID NO: 174) | |

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises a VHCDR1 amino acid sequence set forth in table 6;
VHCDR2 comprises a VHCDR2 amino acid sequence set forth in table 6;
VHCDR3 comprises a VHCDR3 amino acid sequence set forth in table 6;
VLCDR1 comprises a VLCDR1 amino acid sequence set forth in table 6;
VLCDR2 comprises a VLCDR2 amino acid sequence set forth in table 6; and
VLCDR3 comprises a VLCDR3 amino acid sequence set forth in table 6;
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 1 to 49 of SEQ ID NO: 1.

The specific binding molecule may bind to a polypeptide or protein molecule comprising an amino acid sequence comprising residues 1 to 49 of SEQ ID NO: 1 with a K_{D} of less than around 25 nM. The K_{D} may be less than around 20 nM, less than around 15 nM, or less than around 10 nM. The K_{D} may preferably be for binding to SEQ ID NO: 1 or to SEQ ID NO: 148. The K_{D} for binding to SEQ ID NO: 1 may be around 1 nM to around 20 nM. The K_{D} for binding to SEQ ID NO: 1 may be around 1 nM to around 10 nM. The K_{D} for binding to SEQ ID NO: 1 may be around 19.1 nM, optionally wherein the specific binding molecule comprises the CDRs of 3aD3. The K_{D} for binding to SEQ ID NO: 1 may be around 3.6 nM, optionally wherein the specific binding molecule comprises the CDRs of 3aH6. The K_{D} for binding to SEQ ID NO: 1 may be around 6.1 nM, optionally wherein the specific binding molecule comprises the CDRs of 3aG3. The K_{D} for binding to SEQ ID NO: 1 may be around 8.9 nM, optionally wherein the specific binding molecule comprises the CDRs of 3bG4.

The specific binding molecule may comprise an amino acid sequence having at least 70%, at least 75%, at least 80%, at least 90%, at least 95% or at least 99% identity to SEQ ID NO: 422 wherein the specific binding molecule binds to a polypeptide or protein molecule comprising an amino acid sequence comprising residues 1 to 49 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 422. The CDRs may be 100% identical to the CDRs of SEQ ID NO: 422. The specific binding molecule may comprise an amino acid sequence having at least 70%, at least 75%, at least 80%, at least 90%, at least 95% or at least 99% identity to SEQ ID NO: 422 wherein CDRs are 100% identical to the CDRs of SEQ ID NO: 422. The specific binding molecule may comprise the amino acid sequence of SEQ ID NO: 422.

(3aD3 amino acid sequence)
SEQ ID NO: 422
QVQLQESGPSLVKPSQTLSLTCTVSGFSLTSNGVGWVRRAPGKVPEWVG
DISSGGKVYGHPALKSRLSITRDTSKSQVSLSVSSVTSEDTAVYYCVRC
RDGGVSYGYDSDYWGPGLLVTVSSEGKSSGASGESKVDDQAVVTQPSSV
SKSLGQSVSITCSGSSSNVGYGDYVGWFQQVPGSAPKLLIYGVTERASG
VPDRFSGSRSGNTATLTISSIQAEDEADYYCASYDDSSGGIFGSGTRLT
VLG

The specific binding molecule may comprise an amino acid sequence having at least 70%, at least 75%, at least 80%, at least 90%, at least 95% or at least 99% identity to SEQ ID NO: 423 wherein the specific binding molecule binds to a polypeptide or protein molecule comprising an amino acid sequence comprising residues 1 to 49 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 423. The CDRs may be 100% identical to the CDRs of SEQ ID NO: 423. The specific binding molecule may comprise an amino acid sequence having at least 70%, at least 75%, at least 80%, at least 90%, at least 95% or at least 99% identity to SEQ ID NO: 423 wherein CDRs are 100% identical to the CDRs of SEQ ID NO: 423. The specific binding molecule may comprise the amino acid sequence of SEQ ID NO: 423.

(3aH6 amino acid sequence)
SEQ ID NO: 423
QVQLQESGPSLVKPSQTLSLTCTVSGFSLSSNGVGWVRQAPGKVPEWLG
DISSVGKKYANPALKSRLSFTRDTSKSQVSLSLSSVTTEDTAVYYCVKC
RDGGVTYGYDIDYWGPGLLVTASSEGKSSGASGESKVDDQAVVTQPSSV
SGSLGQSVSITCSGSSGNVGYGDYVSWFQQFHGSAPKLLIYGATNLASG
VPARFSGSRSGNTATLTISSLHAEDEADYYCASYDSSSGGVFGSGTRLT
VLG

The specific binding molecule may comprise an amino acid sequence having at least 70%, at least 75%, at least 80%, at least 90%, at least 95% or at least 99% identity to SEQ ID NO: 424 wherein the specific binding molecule binds to a polypeptide or protein molecule comprising an amino acid sequence comprising residues 1 to 49 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 424. The CDRs may be 100% identical to the CDRs of SEQ ID NO: 424. The specific binding molecule may comprise an amino acid sequence having at least 70%, at least 75%, at least 80%, at least 90%, at least 95% or at least 99% identity to SEQ ID NO: 424 wherein CDRs are 100% identical to the CDRs of SEQ ID NO: 424. The specific binding molecule may comprise the amino acid sequence of SEQ ID NO: 424.

(3aG3 amino acid sequence)
SEQ ID NO: 424
QVQLQESGPSLVKPSQTLSLTCTVSGFSLSSNGVGWVRQAPGKVPEWVG
DISSSGKAYANPALKSRLSITRDTAKTQVFLSLSSVTTEDTAVYYCVRC
RDGGVSYGYDIDYWGPGLLVTVSSEGKSSGASGESKVDDQAVLTQPPSV
SGSPGQRVSITCSGSSSNIGGGNYLSWFQQVPGSAPKLLIYGATSRASG
VPDRFSGSRSGNTATLTISSLQAEDEADYYCASFDTSSGGIFGAGTRLT
VLG

The specific binding molecule may comprise an amino acid sequence having at least 70%, at least 75%, at least 80%, at least 90%, at least 95% or at least 99% identity to SEQ ID NO: 425 wherein the specific binding molecule binds to a polypeptide or protein molecule comprising an amino acid sequence comprising residues 1 to 49 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 425. The CDRs may be 100% identical to the CDRs of SEQ ID NO: 425. The specific binding molecule may comprise an amino acid sequence having at least 70%, at least 75%, at least 80%, at least 90%, at least 95% or at least 99% identity to SEQ ID NO: 425 wherein CDRs are 100% identical to the CDRs of SEQ ID NO: 425. The specific binding molecule may comprise the amino acid sequence of SEQ ID NO: 425.

(3bG4 amino acid sequence)
SEQ ID NO: 425
QVQLQESGPSLVKPSQTLSLTCTISGFSLISNGVGWVRQAPGKVPEWVG
DIASSGKAYSNPALKSRLSITRDTSKSQVSLSLRSVTTEDTAVYYCVRC
RDGGVTYGYDIDYWGPGLLVTVSSEGKSSGASGESKVDDQAVLTQPSSV
SKSLGQSVSITCSGSTSNVGSGNDVSWFQQVPGSAPKLLFYGATNRASG
VPDRFSGSRSGNTATLTITSLQAEDEADYYCGSYDSNSGGIFGSGTRLT
VLG

The epitope of the specific binding molecule may be within an amino acid sequence comprising residues 49 to 111 of SEQ ID NO: 1. Accordingly, the epitope may be within the amino acid sequence of SEQ ID NO: 175 (QTPTEDGSEEPGSETSDAKSTPTAEDVTAPLVDEGAPGKQAAAQPHTEIPEGTTAEEAGIGDT).

The epitope may be within an amino acid sequence comprising residues 49 to 111 of SEQ ID NO: 1. This epitope may be bound by the CDRs of the specific binding molecule referred to as “3bF4” herein.

The epitope may comprise the amino acid sequence of SEQ ID NO: 175 (QTPTEDGSEEPGSETSDAKSTPTAEDVTAPLVDEGAPGKQAAAQPHTEIPEGTTAEEAGIGDT). The epitope may comprise an amino acid sequence having at least 70%, at least 75%, at least 80%, at least 90%, at least 95% or at least 99% identity to SEQ ID NO: 175 (QTPTEDGSEEPGSETSDAKSTPTAEDVTAPLVDEGAPGKQAAAQPHTEIPEGTTAEEAGIGDT).

The epitope may consist of the amino acid sequence of SEQ ID NO: 175 (QTPTEDGSEEPGSETSDAKSTPTAEDVTAPLVDEGAPGKQAAAQPHTEIPEGTTAEEAGIGDT). The epitope may consist of an amino acid sequence having at least 70%, at least 75%, at least 80%, at least 90%, at least 95% or at least 99% identity to SEQ ID NO: 175 (QTPTEDGSEEPGSETSDAKSTPTAEDVTAPLVDEGAPGKQAAAQPHTEIPEGTTAEEAGIGDT).

The specific binding molecule may bind to a polypeptide or protein molecule comprising an amino acid sequence comprising residues 49 to 111 of SEQ ID NO: 1. Said specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (S N G V G);
VHCDR2 comprises the sequence set forth in SEQ ID NO: 176 (D I/K S S V/A G K K/T Y A/GN P A L K S);
VHCDR3 comprises the sequence set forth in SEQ ID NO: 177 (C R D G G V T Y G Y D I/V D Y);
VLCDR1 comprises the sequence set forth in SEQ ID NO: 178 (S G S S S N V G L/Y R/G N/D Y/V V T/S);
VLCDR2 comprises the sequence set forth in SEQ ID NO: 179 (G A/T T S/T R A S); and
VLCDR3 comprises the sequence set forth in SEQ ID NO: 180 (A S A/F D T/S N/D D/S G G V/I);
or for each CDR sequence, an amino acid sequence with
   (i) at least 85% identity thereto, and/or
   (ii) one, two, or three amino acid substitutions relative thereto.

Said sequence identity is at least about 85% sequence identity and may therefore be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity. Preferably said sequence identity is at least 90% or at least 95%.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (SNGVG);
VHCDR2 comprises the sequence set forth in SEQ ID NO: 157 (DISSVGKKYANPALKS), or SEQ ID NO: 182 (DKSSAGKTYGNPALKS);
VHCDR3 comprises the sequence set forth in SEQ ID NO: 161 (CRDGGVTYGYDIDY), or SEQ ID NO: 184 (CRDGGVTYGYDVDY);
VLCDR1 comprises the sequence set forth in SEQ ID NO: 185 (SGSSSNVGLRNYVT), or SEQ ID NO: 186 (SGSSSNVGYGDVVS);
VLCDR2 comprises the sequence set forth in SEQ ID NO: 141 (GATSRAS), or SEQ ID NO: 188 (GTTTRAS); and
VLCDR3 comprises the sequence set forth in SEQ ID NO: 189 (ASADTNDGGV), or SEQ ID NO: 190 (ASFDSDSGGI);
or for each CDR sequence, an amino acid sequence with
   (i) at least 85% identity thereto, and/or
   (ii) one, two, or three amino acid substitutions relative thereto,
      wherein the specific binding molecule binds to a polypeptide or protein molecule comprising an amino acid sequence comprising residues 49 to 111 of SEQ ID NO: 1.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (SNGVG);
VHCDR2 comprises the sequence set forth in SEQ ID NO: 157 (DISSVGKKYANPALKS);
VHCDR3 comprises the sequence set forth in SEQ ID NO: 161 (CRDGGVTYGYDIDY);
VLCDR1 comprises the sequence set forth in SEQ ID NO: 185 (SGSSSNVGLRNYVT);
VLCDR2 comprises the sequence set forth in SEQ ID NO: 141 (GATSRAS); and
VLCDR3 comprises the sequence set forth in SEQ ID NO: 189 (ASADTNDGGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 49 to 111 of SEQ ID NO: 1. The specific binding molecule comprising CDRs having 100% identity to those given above is referred to as “3bF4” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (SNGVG);
VHCDR2 comprises the sequence set forth in SEQ ID NO: 157 (DISSVGKKYANPALKS);
VHCDR3 comprises the sequence set forth in SEQ ID NO: 161 (CRDGGVTYGYDIDY);
VLCDR1 comprises the sequence set forth in SEQ ID NO: 186 (SGSSSNVGYGDVVS);
VLCDR2 comprises the sequence set forth in SEQ ID NO: 141 (GATSRAS); and
VLCDR3 comprises the sequence set forth in SEQ ID NO: 189 (ASADTNDGGV).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (SNGVG);
VHCDR2 comprises the sequence set forth in SEQ ID NO: 182 (DKSSAGKTYGNPALKS);
VHCDR3 comprises the sequence set forth in SEQ ID NO: 184 (CRDGGVTYGYDVDY);
VLCDR1 comprises the sequence set forth in SEQ ID NO: 186 (SGSSSNVGYGDVVS);
VLCDR2 comprises the sequence set forth in SEQ ID NO: 188 (GTTTRAS); and
VLCDR3 comprises the sequence set forth in SEQ ID NO: 190 (ASFDSDSGGI);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 49 to 111 of SEQ ID NO: 1. The specific binding molecule comprising CDRs having 100% identity to those given above is referred to as “3aB7” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 149 (SNGVG);
VHCDR2 comprises the sequence set forth in SEQ ID NO: 182 (DKSSAGKTYGNPALKS);
VHCDR3 comprises the sequence set forth in SEQ ID NO: 184 (CRDGGVTYGYDVDY);
VLCDR1 comprises the sequence set forth in SEQ ID NO: 186 (SGSSSNVGYGDVVS);
VLCDR2 comprises the sequence set forth in SEQ ID NO: 188 (GTTTRAS); and
VLCDR3 comprises the sequence set forth in SEQ ID NO: 190 (ASFDSDSGGI).

The specific binding molecule may comprise the CDR sequences of a clone set out in Table 7 below. The epitope may be within residues 49 to 111 of SEQ ID NO: 1.

TABLE 7

| Clone | VH | | | VL | | | |
|---|---|---|---|---|---|---|---|
| name | CDR1 | CDR2 | CDR3 | CDR1 | CDR2 | CDR3 | Epitope |
| 3aB7 | SNGVG | DKSSAGKTYGNP | CRDGGVTYGYDVDY | SGSSSNVGYGDVVS | GTTTRAS | ASFDSDSGGI | 49-111 |
| | (SEQ ID | ALKS | (SEQ ID | (SEQ ID | (SEQ ID | (SEQ ID | |
| | NO: 149) | (SEQ ID | NO: 184) | NO: 186) | NO: 188) | NO: 190) | |
| | | NO: 182) | | | | | |
| 3bF4 | SNGVG | DISSVGKKYANP | CRDGGVTYGYDIDY | SGSSSNVGLRNYVT | GATSRAS | ASADTNDGGV | 49-111 |
| | (SEQ ID | ALKS | (SEQ ID | (SEQ ID | (SEQ ID | (SEQ ID | |
| | NO: 149) | (SEQ ID | NO: 161) | NO: 185) | NO: 141) | NO: 189) | |
| | | NO: 157) | | | | | |

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises a VHCDR1 amino acid sequence set forth in table 7;
VHCDR2 comprises a VHCDR2 amino acid sequence set forth in table 7;
VHCDR3 comprises a VHCDR3 amino acid sequence set forth in table 7;
VLCDR1 comprises a VLCDR1 amino acid sequence set forth in table 7;
VLCDR2 comprises a VLCDR2 amino acid sequence set forth in table 7; and
VLCDR3 comprises a VLCDR3 amino acid sequence set forth in table 7;
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 49 to 111 of SEQ ID NO: 1.

The specific binding molecule may bind to a polypeptide or protein molecule comprising an amino acid sequence comprising residues 49 to 111 of SEQ ID NO: 1 with a K_{D} of less than around 250 nM. The K_{D} may be less than around 200 nM, less than around 150 nM, or less than around 100 nM. The K_{D} may preferably be for binding to SEQ ID NO: 1 or to SEQ ID NO: 175. The K_{D} for binding to SEQ ID NO: 1 may be around 1 nM to around 20 nM. The K_{D} for binding to SEQ ID NO: 1 may be around 50 nM to around 150 nM. The K_{D} for binding to SEQ ID NO: 1 may be around 69 nM, optionally wherein the specific binding molecule comprises the CDRs of 3aB7. The K_{D} for binding to SEQ ID NO: 1 may be around 140 nM, optionally wherein the specific binding molecule comprises the CDRs of 3bF4.

The specific binding molecule may comprise an amino acid sequence having at least 70%, at least 75%, at least 80%, at least 90%, at least 95% or at least 99% identity to SEQ ID NO: 420 wherein the specific binding molecule binds to a polypeptide or protein molecule comprising an amino acid sequence comprising residues 49 to 111 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 420. The CDRs may be 100% identical to the CDRs of SEQ ID NO: 420. The specific binding molecule may comprise an amino acid sequence having at least 70%, at least 75%, at least 80%, at least 90%, at least 95% or at least 99% identity to SEQ ID NO: 420 wherein CDRs are 100% identical to the CDRs of SEQ ID NO: 420. The specific binding molecule may comprise the amino acid sequence of SEQ ID NO: 420.

(3aB7 amino acid sequence)
SEQ ID NO: 420
QVQLQESGPSLVKPSQTLSLTCTVSGFSLTSNGVGWVRQAPGKVPEWVG
DKSSAGKTYGNPALKSRLSITRDTSKSQVSLSLSSVTTEDTAVYYCVRC
RDGGVTYGYDVDYWGPGLLVTVSSEGKSSGASGESKVDDQAVLTQPSSV
SKSLGQSVSITCSGSSSNVGYGDVVSWFQQFPGSAPKLLIFGTTTRASG
VPDRFSGSRSGNAATLTINSLQAEDEADYYCASFDSDSGGIAGSGTRLT
VLG

The specific binding molecule may comprise an amino acid sequence having at least 70%, at least 75%, at least 80%, at least 90%, at least 95% or at least 99% identity to SEQ ID NO: 421 wherein the specific binding molecule binds to a polypeptide or protein molecule comprising an amino acid sequence comprising residues 49 to 111 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 421. The CDRs may be 100% identical to the CDRs of SEQ ID NO: 421. The specific binding molecule may comprise an amino acid sequence having at least 70%, at least 75%, at least 80%, at least 90%, at least 95% or at least 99% identity to SEQ ID NO: 421 wherein CDRs are 100% identical to the CDRs of SEQ ID NO: 421. The specific binding molecule may comprise the amino acid sequence of SEQ ID NO: 421.

(3bF4 amino acid sequence)
SEQ ID NO: 421
QVQLQESGPSLVKPSQTLSLTCTVSGFSLSSNGVGWVRQAPGKVPE
WLGDISSVGKKYANPALKSRLSFTRDTSKSQVSLSLSSVTTEDTA
VYYCVKCRDGGVTYGYDIDYWGPGLLVTVSSEGKSSGASGESKVD
DQAVLTQPSSVSKSTGQTVSITCSGSSSNVGLRNYVTWFQQVPGS
APKLLIYGATSRASGIPDRFSGSRSGNTATLIISSLQAEDEADYY
CASADTNDGGVFGSGTRLTVLG

The epitope of the specific binding molecule may be within an amino acid sequence comprising residues 146 to 157 of SEQ ID NO: 1. Accordingly, the epitope may be within the amino acid sequence of SEQ ID NO: 191 (GKTKIATPRGA).

The epitope may be within an amino acid sequence comprising residues 147 to 157 of SEQ ID NO: 1. This epitope may be bound by the CDRs of the specific binding molecule referred to as “3aD6” herein.

The epitope may comprise the amino acid sequence of SEQ ID NO: 191 (GKTKIATPRGA). The epitope may comprise an amino acid sequence having at least 70%, at least 75%, at least 80%, at least 90%, at least 95% or at least 99% identity to SEQ ID NO: 191 (GKTKIATPRGA).

The epitope may consist of the amino acid sequence of SEQ ID NO: 191 (GKTKIATPRGA). The epitope may consist of an amino acid sequence having at least 70%, at least 75%, at least 80%, at least 90%, at least 95% or at least 99% identity to SEQ ID NO: 191 (GKTKIATPRGA).

The specific binding molecule may bind to a polypeptide or protein molecule comprising an amino acid sequence comprising residues 147 to 157 of SEQ ID NO: 1. Said specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 192 (S N A V I/G);
VHCDR2 comprises the sequence set forth in SEQ ID NO: 193 (L I D V/I D G D A/T A Y D/N P A L K/E S);
VHCDR3 comprises the sequence set forth in SEQ ID NO: 194 (D/H Y G/D S/K W G Y V/A S/D D/S I D Y);
VLCDR1 comprises the sequence set forth in SEQ ID NO: 195 (S G S D/S-/S-/N-/V I/G G/Y G A/D D/Y V G);
VLCDR2 comprises the sequence set forth in SEQ ID NO: 196 (D N/A D/T N/T R P/A S); and
VLCDR3 comprises the sequence set forth in SEQ ID NO: 197 (G/A T/S Y S/Q G/N A/E N/R Y/S G I/V);
or for each CDR sequence, an amino acid sequence with
   (i) at least 85% identity thereto, and/or
   (ii) one, two, or three amino acid substitutions relative thereto.

Said sequence identity is at least about 85% sequence identity and may therefore be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity. Preferably said sequence identity is at least 90% or at least 95%.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 198 (SNAVI), or SEQ ID NO: 17 (SNAVG);
VHCDR2 comprises the sequence set forth in SEQ ID NO: 200 (LIDVDGDAAYDPALKS), or
SEQ ID NO: 201 (LIDIDGDTAYNPALES);
VHCDR3 comprises the sequence set forth in SEQ ID NO: 202 (DYGSWGYVSDIDY), or SEQ ID NO: 203 (HYDKWGYADSIDY);
VLCDR1 comprises the sequence set forth in SEQ ID NO: 204 (SGSDIGGADVG), or SEQ ID NO: 138 (SGSSSNVGYGDYVG);
VLCDR2 comprises the sequence set forth in SEQ ID NO: 206 (DNDNRPS), or SEQ ID NO: 207 (DATTRAS); and
VLCDR3 comprises the sequence set forth in SEQ ID NO: 208 (GTYSGANYGI), or SEQ ID NO: 209 (ASYQNERSGV);
or for each CDR sequence, an amino acid sequence with
   (i) at least 85% identity thereto, and/or
   (ii) one, two, or three amino acid substitutions relative thereto,
   wherein the specific binding molecule binds to a polypeptide or protein molecule comprising an amino acid sequence comprising residues 147 to 157 of SEQ ID NO: 1.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 198 (SNAVI);
VHCDR2 comprises the sequence set forth in SEQ ID NO: 200 (LIDVDGDAAYDPALKS);
VHCDR3 comprises the sequence set forth in SEQ ID NO: 202 (DYGSWGYVSDIDY);
VLCDR1 comprises the sequence set forth in SEQ ID NO: 204 (SGSDIGGADVG);
VLCDR2 comprises the sequence set forth in SEQ ID NO: 206 (DNDNRPS); and
VLCDR3 comprises the sequence set forth in SEQ ID NO: 208 (GTYSGANYGI);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 147 to 157 of SEQ ID NO: 1. The specific binding molecule comprising CDRs having 100% identity to those given above is referred to as “3aD6” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 198 (SNAVI);
VHCDR2 comprises the sequence set forth in SEQ ID NO: 200 (LIDVDGDAAYDPALKS);
VHCDR3 comprises the sequence set forth in SEQ ID NO: 202 (DYGSWGYVSDIDY);
VLCDR1 comprises the sequence set forth in SEQ ID NO: 204 (SGSDIGGADVG);
VLCDR2 comprises the sequence set forth in SEQ ID NO: 206 (DNDNRPS); and
VLCDR3 comprises the sequence set forth in SEQ ID NO: 208 (GTYSGANYGI).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 17 (SNAVG);
VHCDR2 comprises the sequence set forth in SEQ ID NO: 201 (LIDIDGDTAYNPALES);
VHCDR3 comprises the sequence set forth in SEQ ID NO: 203 (HYDKWGYADSIDY);
VLCDR1 comprises the sequence set forth in SEQ ID NO: 138 (SGSSSNVGYGDYVG);
VLCDR2 comprises the sequence set forth in SEQ ID NO: 207 (DATTRAS); and
VLCDR3 comprises the sequence set forth in SEQ ID NO: 209 (ASYQNERSGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 147 to 157 of SEQ ID NO: 1. The specific binding molecule comprising CDRs having 100% identity to those given above is referred to as “3aA6” herein.

The specific binding molecule may comprise the CDR sequences of a clone set out in Table 8 below. The epitope may be within residues 147 to 157 of SEQ ID NO: 1.

TABLE 8

| Clone | VH | | | VL | | | |
|---|---|---|---|---|---|---|---|
| name | CDR1 | CDR2 | CDR3 | CDR1 | CDR2 | CDR3 | Epitope |
| 3aA6 | SNAV | LIDI | HYDK | SGSS | DATT | ASYQ | 147-157 |
| | G | DGDT | WGYA | SNVG | RAS | NERS | |
| | (SEQ | AYNP | DSID | YGDY | (SEQ | GV | |
| | ID | ALES | Y | VG | ID | (SEQ | |
| | NO: | (SEQ | (SEQ | (SEQ | NO: | ID | |
| | 17) | ID | ID | ID | 207) | NO: | |
| | | NO: | NO: | NO: | | 209) | |
| | | 201) | 203) | 138) | | | |
| 3aD6 | SNAV | LIDV | DYGS | SGSD | DNDN | GTYS | 147-157 |
| | I | DGDA | WGYV | --- | RPS | GANY | |
| | (SEQ | AYDP | SDID | IGGA | (SEQ | GI | |
| | ID | ALKS | Y | DVG | ID | (SEQ | |
| | NO: | (SEQ | (SEQ | (SEQ | NO: | ID | |
| | 198) | ID | ID | ID | 206) | NO: | |
| | | NO: | NO: | NO: | | 208) | |
| | | 200) | 202) | 204) | | | |

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises a VHCDR1 amino acid sequence set forth in table 8;
VHCDR2 comprises a VHCDR2 amino acid sequence set forth in table 8;
VHCDR3 comprises a VHCDR3 amino acid sequence set forth in table 8;
VLCDR1 comprises a VLCDR1 amino acid sequence set forth in table 8;
VLCDR2 comprises a VLCDR2 amino acid sequence set forth in table 8; and
VLCDR3 comprises a VLCDR3 amino acid sequence set forth in table 8;
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 147 to 157 of SEQ ID NO: 1.

The specific binding molecule may bind to a polypeptide or protein molecule comprising an amino acid sequence comprising residues 147 to 157 of SEQ ID NO: 1 with a K_{D} of less than around 50 nM. The K_{D} may be less than around 40 nM, less than around 30 nM, or less than around 20 nM. The K_{D} may preferably be for binding to SEQ ID NO: 1 or to SEQ ID NO: 191. The K_{D} for binding to SEQ ID NO: 1 may be around 10 nM to around 20 nM. The K_{D} for binding to SEQ ID NO: 1 may be around 16.5 nM, optionally wherein the specific binding molecule comprises the CDRs of 3aD6.

The specific binding molecule may comprise an amino acid sequence having at least 70%, at least 75%, at least 80%, at least 90%, at least 95% or at least 99% identity to SEQ ID NO: 418 wherein the specific binding molecule binds to a polypeptide or protein molecule comprising an amino acid sequence comprising residues 147 to 157 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 418. The CDRs may be 100% identical to the CDRs of SEQ ID NO: 418. The specific binding molecule may comprise an amino acid sequence having at least 70%, at least 75%, at least 80%, at least 90%, at least 95% or at least 99% identity to SEQ ID NO: 418 wherein CDRs are 100% identical to the CDRs of SEQ ID NO: 418. The specific binding molecule may comprise the amino acid sequence of SEQ ID NO: 418.

(3aA6 amino acid sequence)
SEQ ID NO: 418
QVRLQESGSSLVKPSQTLSLVCTVSGFPLTSNAVGWVRQAPGKAP
EWLGLIDIDGDTAYNPALESRLSITRDTSKSQVSLSLSSVAIEDT
AVYYCARHYDKWGYADSIDYWGPGLLVTVSSEGKSSGASGESKVD
DQALLTQPSSVFGSLGQRVSITCSGSSSNVGYGDYVGWYQQVPGS
APKLLIYDATTRASGVPDRFSGSRSGNTATLTISSLQAEDEADYY
CASYQNERSGVFGSGTRLTVLG

The specific binding molecule may comprise an amino acid sequence having at least 70%, at least 75%, at least 80%, at least 90%, at least 95% or at least 99% identity to SEQ ID NO: 419 wherein the specific binding molecule binds to a polypeptide or protein molecule comprising an amino acid sequence comprising residues 147 to 157 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 419. The CDRs may be 100% identical to the CDRs of SEQ ID NO: 419. The specific binding molecule may comprise an amino acid sequence having at least 70%, at least 75%, at least 80%, at least 90%, at least 95% or at least 99% identity to SEQ ID NO: 419 wherein CDRs are 100% identical to the CDRs of SEQ ID NO: 419. The specific binding molecule may comprise the amino acid sequence of SEQ ID NO: 419.

(3aD6 amino acid sequence)
SEQ ID NO: 419
QVQLQESGPSLVKPSQTLSLTCTVSGFSLTSNAVIWVRQAPGKAP
EWVALIDVDGDAAYDPALKSRLSITRDTSKSQVSLSLRSVTTEDT
AVYYCARDYGSWGYVSDIDYWGPGLLVTVSSEGKSSGASGESKVD
DQAVLTQPSSVSGSLGQRVSITCSGSDIGGADVGWFQQVPGSGLR
TLIYDNDNRPSGVPDRFSGSKSGNTATLTISSLQPEDEADYFCGT
YSGANYGIFGSGTRLTVLG

The epitope of the specific binding molecule may be within an amino acid sequence comprising residues 379 to 391 of SEQ ID NO: 1. Accordingly, the epitope may be within the amino acid sequence of SEQ ID NO: 221 (RENAKAKTDHGAE).

The epitope may comprise the amino acid sequence of SEQ ID NO: 221 (RENAKAKTDHGAE). The epitope may comprise an amino acid sequence having at least 70%, at least 75%, at least 80%, at least 90%, at least 95% or at least 99% identity to SEQ ID NO: 221 (RENAKAKTDHGAE). The epitope may be within an amino acid sequence comprising residues 379 to 391 of SEQ ID NO: 1. This epitope may be bound by the CDRs of the specific binding molecule referred to as “E2E8” herein. Critical residues of the epitope may be residue 391 (E) (numbering according to SEQ ID NO: 1). The epitope may comprise the amino acid sequence of SEQ ID NO: 222 (XXXXXXXXXXXXE, wherein X is any amino acid). The epitope may comprise the amino acid sequence of SEQ ID NO: 8, wherein any one or more residue other than residue number 391 (E) is replaced by a non-conservative amino acid substitution (numbering according to SEQ ID NO: 1). The epitope may comprise the amino acid sequence of SEQ ID NO: 221, wherein any one or more residue other than residue number 391 (E) is replaced by a conservative amino acid substitution (numbering according to SEQ ID NO: 1). The epitope may comprise the amino acid sequence of SEQ ID NO: 221, wherein any one or more residue other than residue number 391 (E) is replaced by a conservative amino acid substitution (numbering according to SEQ ID NO: 1) and any one or more residue other than residue number 391 (E) is replaced by a non-conservative amino acid substitution (numbering according to SEQ ID NO: 1).

The epitope may comprise the amino acid sequence of SEQ ID NO: 221 (RENAKAKTDHGAE). The epitope may comprise an amino acid sequence having at least 70%, at least 75%, at least 80%, at least 90%, at least 95% or at least 99% identity to SEQ ID NO: 221 (RENAKAKTDHGAE).

The epitope may consist of the amino acid sequence of SEQ ID NO: 221 (RENAKAKTDHGAE). The epitope may consist of an amino acid sequence having at least 70%, at least 75%, at least 80%, at least 90%, at least 95% or at least 99% identity to SEQ ID NO: 221 (RENAKAKTDHGAE).

The specific binding molecule may bind to a polypeptide or protein molecule comprising an amino acid sequence comprising residues 379 to 391 of SEQ ID NO: 1. Said specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 223 (D/S R/W G V A);
VHCDR2 comprises the sequence set forth in SEQ ID NO: 224 (T M R S G G T/G I/T D/E Y/D N P A L K S);
VHCDR3 comprises the sequence set forth SEQ ID NO: 225 (G Y L S G D/I/V R/H Y A);
VLCDR1 comprises the sequence set forth in SEQ ID NO: 226 (S G S R/S S D/N I/V G Y/D/A G N/D/R Y V S/G);
VLCDR2 comprises the sequence set forth in SEQ ID NO: 227 (D/S/G T/A N/R/T T/N/S R A S); and
VLCDR3 comprises the sequence set forth in SEQ ID NO: 228 (A N/S I D S/T S/G R/N S/N H/L L/I);
or for each CDR sequence, an amino acid sequence with
   (i) at least 85% identity thereto, and/or
   (ii) one, two, or three amino acid substitutions relative thereto.

Said sequence identity is at least about 85% sequence identity and may therefore be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity. Preferably said sequence identity is at least 90% or at least 95%.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 229 (DRGVA), SEQ ID NO: 230 (DWGVA), or SEQ ID NO: 231 (SWGVA);
VHCDR2 comprises the sequence set forth in SEQ ID NO: 232 (TMRSGGTIDYNPALKS), SEQ ID NO: 233 (TMRSGGGTEYNPALKS), or SEQ ID NO: 234 (TMRSGGTTDDNPALKS);
VHCDR3 comprises the sequence set forth in SEQ ID NO: 235 (GYLSGDRYA), SEQ ID NO: 236 (GYLSGIHYA), or SEQ ID NO: 237 (GYLSGVHYA);
VLCDR1 comprises the sequence set forth in SEQ ID NO: 238 (SGSRSDIGYGNYVS), SEQ ID NO: 239 (SGSSSNVGAGNYVG), SEQ ID NO: 240 (SGSSSNVGDGDYVG), or SEQ ID NO: 241 (SGSSSNVGDGRYVS);
VLCDR2 comprises the sequence set forth in SEQ ID NO: 242 (DTNTRAS), SEQ ID NO: 243 (DTTSRAS), SEQ ID NO: 170 (GATNRAS), or SEQ ID NO: 244 (SARNRAS); and
VLCDR3 comprises the sequence set forth in SEQ ID NO: 245 (ANIDSSRSHL), SEQ ID NO: 246 (ASIDSGNNLL), or SEQ ID NO: 247 (ASIDTSRSHI);
or for each CDR sequence, an amino acid sequence with
   (i) at least 85% identity thereto, and/or
   (ii) one, two, or three amino acid substitutions relative thereto,
   wherein the specific binding molecule binds to a polypeptide or protein molecule comprising an amino acid sequence comprising residues 379 to 391 of SEQ ID NO: 1.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 229 (DRGVA);
VHCDR2 comprises the sequence set forth in SEQ ID NO: 232 (TMRSGGTIDYNPALKS);
VHCDR3 comprises the sequence set forth in SEQ ID NO: 235 (GYLSGDRYA);
VLCDR1 comprises the sequence set forth in SEQ ID NO: 238 (SGSRSDIGYGNYVS);
VLCDR2 comprises the sequence set forth in SEQ ID NO: 242 (DTNTRAS); and
VLCDR3 comprises the sequence set forth in SEQ ID NO: 245 (ANIDSSRSHL);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 379 to 391 of SEQ ID NO: 1. The specific binding molecule comprising CDRs having 100% identity to those given above is referred to as “E2E8” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 229 (DRGVA);
VHCDR2 comprises the sequence set forth in SEQ ID NO: 232 (TMRSGGTIDYNPALKS);
VHCDR3 comprises the sequence set forth in SEQ ID NO: 235 (GYLSGDRYA);
VLCDR1 comprises the sequence set forth in SEQ ID NO: 238 (SGSRSDIGYGNYVS);
VLCDR2 comprises the sequence set forth in SEQ ID NO: 242 (DTNTRAS); and
VLCDR3 comprises the sequence set forth in SEQ ID NO: 245 (ANIDSSRSHL).

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 483 (QVQLQESGPSLVKPSQTLSLTCTVSGFSLT);
VHFR2 comprises the sequence set forth in SEQ ID NO: 484 (WVRQAPGKALEWVG);
VHFR3 comprises the sequence set forth in SEQ ID NO: 485 (RLSITRDTSKSQVFLSLSSVTTEDMAMYYCAR);
VHFR4 comprises the sequence set forth in SEQ ID NO: 486 (WGRGLLVTVSS);
VLFR1 comprises the sequence set forth in SEQ ID NO: 487 (QAVLTQPSSVSKSLGQSVSIAC);
VLFR2 comprises the sequence set forth in SEQ ID NO: 488 (WFQQIPGSAPKLLIY);
VLFR3 comprises the sequence set forth in SEQ ID NO: 489 (GVPDRFSGARSGNTATLTINSLQAEDEADYYC);
VLFR4 comprises the sequence set forth in SEQ ID NO: 490 (FGSGTRLTVLG);
or for each FR sequence, an amino acid sequence with
   (i) at least 50% identity thereto, and/or
   (ii) one, two, three, four or five amino acid substitutions relative thereto.

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 483 (QVQLQESGPSLVKPSQTLSLTCTVSGFSLT);
VHFR2 comprises the sequence set forth in SEQ ID NO: 484 (WVRQAPGKALEWVG);
VHFR3 comprises the sequence set forth in SEQ ID NO: 485 (RLSITRDTSKSQVFLSLSSVTTEDMAMYYCAR);
VHFR4 comprises the sequence set forth in SEQ ID NO: 486 (WGRGLLVTVSS);
VLFR1 comprises the sequence set forth in SEQ ID NO: 487 (QAVLTQPSSVSKSLGQSVSIAC);
VLFR2 comprises the sequence set forth in SEQ ID NO: 488 (WFQQIPGSAPKLLIY);
VLFR3 comprises the sequence set forth in SEQ ID NO: 489 (GVPDRFSGARSGNTATLTINSLQAEDEADYYC);
VLFR4 comprises the sequence set forth in SEQ ID NO: 490 (FGSGTRLTVLG);
or for each FR sequence, an amino acid sequence with
   (i) at least 50% identity thereto, and/or
   (ii) one, two, three, four or five amino acid substitutions relative thereto,
wherein the specific binding molecule binds to a polypeptide or protein molecule comprising an amino acid sequence comprising residues 379 to 391 of SEQ ID NO: 1. A specific binding molecule comprising FRs having 100% identity to those given above is referred to as “E2E8” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 483 (QVQLQESGPSLVKPSQTLSLTCTVSGFSLT);
   VHFR2 comprises the sequence set forth in SEQ ID NO: 484 (WVRQAPGKALEWVG);
   VHFR3 comprises the sequence set forth in SEQ ID NO: 485 (RLSITRDTSKSQVFLSLSSVTTEDMAMYYCAR);
   VHFR4 comprises the sequence set forth in SEQ ID NO: 486 (WGRGLLVTVSS);
   VLFR1 comprises the sequence set forth in SEQ ID NO: 487 (QAVLTQPSSVSKSLGQSVSIAC);
   VLFR2 comprises the sequence set forth in SEQ ID NO: 488 (WFQQIPGSAPKLLIY);
   VLFR3 comprises the sequence set forth in SEQ ID NO: 489 (GVPDRFSGARSGNTATLTINSLQAEDEADYYC);
   VLFR4 comprises the sequence set forth in SEQ ID NO: 490 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 229 (DRGVA);
   VHCDR2 comprises the sequence set forth in SEQ ID NO: 232 (TMRSGGTIDYNPALKS);
   VHCDR3 comprises the sequence set forth in SEQ ID NO: 235 (GYLSGDRYA);
   VLCDR1 comprises the sequence set forth in SEQ ID NO: 238 (SGSRSDIGYGNYVS);
   VLCDR2 comprises the sequence set forth in SEQ ID NO: 242 (DTNTRAS); and
   VLCDR3 comprises the sequence set forth in SEQ ID NO: 245 (ANIDSSRSHL);
   or for each CDR sequence, an amino acid sequence with
      (i) at least 85% identity thereto, and/or
      (ii) one, two, or three amino acid substitutions relative thereto
   wherein the specific binding molecule binds to a polypeptide or protein molecule comprising an amino acid sequence comprising residues 379 to 391 of SEQ ID NO: 1. The specific binding molecule comprising FRs and CDRs having 100% identity to those given above is referred to as “E2E8” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 483 (QVQLQESGPSLVKPSQTLSLTCTVSGFSLT);
   VHFR2 comprises the sequence set forth in SEQ ID NO: 484 (WVRQAPGKALEWVG);
   VHFR3 comprises the sequence set forth in SEQ ID NO: 485 (RLSITRDTSKSQVFLSLSSVTTEDMAMYYCAR);
   VHFR4 comprises the sequence set forth in SEQ ID NO: 486 (WGRGLLVTVSS);
   VLFR1 comprises the sequence set forth in SEQ ID NO: 487 (QAVLTQPSSVSKSLGQSVSIAC);
   VLFR2 comprises the sequence set forth in SEQ ID NO: 488 (WFQQIPGSAPKLLIY);
   VLFR3 comprises the sequence set forth in SEQ ID NO: 489 (GVPDRFSGARSGNTATLTINSLQAEDEADYYC);
   VLFR4 comprises the sequence set forth in SEQ ID NO: 490 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 229 (DRGVA);
   VHCDR2 comprises the sequence set forth in SEQ ID NO: 232 (TMRSGGTIDYNPALKS);
   VHCDR3 comprises the sequence set forth in SEQ ID NO: 235 (GYLSGDRYA);
   VLCDR1 comprises the sequence set forth in SEQ ID NO: 238 (SGSRSDIGYGNYVS);
   VLCDR2 comprises the sequence set forth in SEQ ID NO: 242 (DTNTRAS); and
   VLCDR3 comprises the sequence set forth in SEQ ID NO: 245 (ANIDSSRSHL);
   wherein the specific binding molecule binds to a polypeptide or protein molecule comprising an amino acid sequence comprising residues 379 to 391 of SEQ ID NO: 1. The specific binding molecule comprising FRs and CDRs having 100% identity to those given above is referred to as “E2E8” herein.

The specific binding molecule may comprise:
(a) A VH domain comprising the sequence set forth in SEQ ID NO: 491 (QVQLQESGPSLVKPSQTLSLTCTVSGFSLTDRGVAWVRQAPGKALEWVGTMRSGGTID YNPALKSRLSITRDTSKSQVFLSLSSVTTEDMAMYYCARGYLSGDRYAWGRGLLVTVSS); and/or
(b) a VL domain comprising the sequence set forth in SEQ ID NO: 492 (QAVLTQPSSVSKSLGQSVSIACSGSRSDIGYGNYVSWFQQIPGSAPKLLIYDTNTRASGV PDRFSGARSGNTATLTINSLQAEDEADYYCANIDSSRSHLFGSGTRLTVLG);
or a humanized variant thereof.

The specific binding molecule may comprise:
(a) A heavy chain comprising the sequence set forth in SEQ ID NO: 493 (QVQLQESGPSLVKPSQTLSLTCTVSGFSLTDRGVAWVRQAPGKALEWVGTMRSGGTID YNPALKSRLSITRDTSKSQVFLSLSSVTTEDMAMYYCARGYLSGDRYAWGRGLLVTVSSA KTTAPSVYPLAPVCGDTTGSSVTLGCLVKGYFPEPVTLTWNSGSLSSGVHTFPAVLQSDL YTLSSSVTVTSSTWPSQSITCNVAHPASSTKVDKKIEPRGPTIKPCPPCKCPAPNLLGGPS VFIFPPKIKDVLMISLSPIVTCVVVDVSEDDPDVQISWFVNNVEVHTAQTQTHREDYNSTLR VVSALPIQHQDWMSGKEFKCKVNNKDLPAPIERTISKPKGSVRAPQVYVLPPPEEEMTKK QVTLTCMVTDFMPEDIYVEWTNNGKTELNYKNTEPVLDSDGSYFMYSKLRVEKKNWVER NSYSCSVVHEGLHNHHTTKSFSRTPGK); and/or
(b) a light chain comprising the sequence set forth in SEQ ID NO: 494 (QAVLTQPSSVSKSLGQSVSIACSGSRSDIGYGNYVSWFQQIPGSAPKLLIYDTNTRASGV PDRFSGARSGNTATLTINSLQAEDEADYYCANIDSSRSHLFGSGTRLTVLGGQPKSSPSV TLFPPSSEELETNKATLVCTITDFYPGVVTVDWKVDGTPVTQGMETTQPSKQSNNKYMAS SYLTLTARAWERHSSYSCQVTHEGHTVEKSLSRADCS);
or a humanized variant thereof.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 230 (DWGVA);
VHCDR2 comprises the sequence set forth in SEQ ID NO: 234 (TMRSGGTTDDNPALKS);
VHCDR3 comprises the sequence set forth in SEQ ID NO: 237 (GYLSGVHYA);
VLCDR1 comprises the sequence set forth in SEQ ID NO: 241 (SGSSSNVGDGRYVS);
VLCDR2 comprises the sequence set forth in SEQ ID NO: 243 (DTTSRAS); and
VLCDR3 comprises the sequence set forth in SEQ ID NO: 246 (ASIDSGNNLL);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 379 to 391 of SEQ ID NO: 1. The specific binding molecule comprising CDRs having 100% identity to those given above is referred to as “E1E8” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 230 (DWGVA);
VHCDR2 comprises the sequence set forth in SEQ ID NO: 234 (TMRSGGTTDDNPALKS);
VHCDR3 comprises the sequence set forth in SEQ ID NO: 237 (GYLSGVHYA);
VLCDR1 comprises the sequence set forth in SEQ ID NO: 241 (SGSSSNVGDGRYVS);
VLCDR2 comprises the sequence set forth in SEQ ID NO: 243 (DTTSRAS); and
VLCDR3 comprises the sequence set forth in SEQ ID NO: 246 (ASIDSGNNLL).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 230 (DWGVA);
VHCDR2 comprises the sequence set forth in SEQ ID NO: 234 (TMRSGGTTDDNPALKS);
VHCDR3 comprises the sequence set forth in SEQ ID NO: 237 (GYLSGVHYA);
VLCDR1 comprises the sequence set forth in SEQ ID NO: 239 (SGSSSNVGAGNYVG);
VLCDR2 comprises the sequence set forth in SEQ ID NO: 70 (GATNRAS); and
VLCDR3 comprises the sequence set forth in SEQ ID NO: 247 (ASIDTSRSHI);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 379 to 391 of SEQ ID NO: 1. The specific binding molecule comprising CDRs having 100% identity to those given above is referred to as “E2A6” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 230 (DWGVA);
VHCDR2 comprises the sequence set forth in SEQ ID NO: 234 (TMRSGGTTDDNPALKS);
VHCDR3 comprises the sequence set forth in SEQ ID NO: 237 (GYLSGVHYA);
VLCDR1 comprises the sequence set forth in SEQ ID NO: 239 (SGSSSNVGAGNYVG);
VLCDR2 comprises the sequence set forth in SEQ ID NO: 70 (GATNRAS); and
VLCDR3 comprises the sequence set forth in SEQ ID NO: 247 (ASIDTSRSHI).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 231 (SWGVA);
VHCDR2 comprises the sequence set forth in SEQ ID NO: 233 (TMRSGGGTEYNPALKS);
VHCDR3 comprises the sequence set forth in SEQ ID NO: 236 (GYLSGIHYA);
VLCDR1 comprises the sequence set forth in SEQ ID NO: 240 (SGSSSNVGDGDYVG);
VLCDR2 comprises the sequence set forth in SEQ ID NO: 244 (SARNRAS); and
VLCDR3 comprises the sequence set forth in SEQ ID NO: 247 (ASIDTSRSHI);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 379 to 391 of SEQ ID NO: 1. The specific binding molecule comprising CDRs having 100% identity to those given above is referred to as “E2B7” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 231 (SWGVA);
VHCDR2 comprises the sequence set forth in SEQ ID NO: 233 (TMRSGGGTEYNPALKS);
VHCDR3 comprises the sequence set forth in SEQ ID NO: 236 (GYLSGIHYA);
VLCDR1 comprises the sequence set forth in SEQ ID NO: 240 (SGSSSNVGDGDYVG);
VLCDR2 comprises the sequence set forth in SEQ ID NO: 244 (SARNRAS); and
VLCDR3 comprises the sequence set forth in SEQ ID NO: 247 (ASIDTSRSHI).

The specific binding molecule may comprise the CDR sequences of a clone set out in Table 9 below. The epitope may be within residues 379 to 391 of SEQ ID NO: 1.

TABLE 9

| | VH | | | VL | | | |
|---|---|---|---|---|---|---|---|
| Clone name | CDR1 | CDR2 | CDR3 | CDR1 | CDR2 | CDR3 | Epitope |
| E1E8 | DWGV | TMRS | GYLS | SGSS | DTTS | ASID | 391‘E’ |
| | A | GGTT | GVHY | SNVG | RAS | SGNN | |
| | (SEQ | DDNP | A | DGRY | (SEQ | LL | |
| | ID | ALKS | (SEQ | VS | ID | (SEQ | |
| | NO: | (SEQ | ID | (SEQ | NO: | ID | |
| | 230) | ID | NO: | ID | 243) | NO: | |
| | | NO: | 237) | NO: | | 246) | |
| | | 234) | | 241) | | | |
| E2A6 | DWGV | TMRS | GYLS | SGSS | GATN | ASID | 391‘E’ |
| | A | GGTT | GVHY | SNVG | RAS | TSRS | |
| | (SEQ | DDNP | A | AGNY | (SEQ | HI | |
| | ID | ALKS | (SEQ | VG | ID | (SEQ | |
| | NO: | (SEQ | ID | (SEQ | NO: | ID | |
| | 230) | ID | NO: | ID | 70) | NO: | |
| | | NO: | 237) | NO: | | 247) | |
| | | 234) | | 239) | | | |
| E2B7 | SWGV | TMRS | GYLS | SGSS | SARN | ASID | 391‘E’ |
| | A | GGGT | GIHY | SNVG | RAS | TSRS | |
| | (SEQ | EYNP | A | DGDY | (SEQ | HI | |
| | ID | ALKS | (SEQ | VG | ID | (SEQ | |
| | NO: | (SEQ | ID | (SEQ | NO: | ID | |
| | 231) | ID | NO: | ID | 244) | NO: | |
| | | NO: | 236) | NO: | | 247) | |
| | | 233) | | 240) | | | |
| E2E8 | DRGV | TMRS | GYLS | SGSR | DTNT | ANID | 391‘E’ |
| | A | GGTI | GDRY | SDIG | RAS | SSRS | |
| | (SEQ | DYNP | A | YGNY | (SEQ | HL | |
| | ID | ALKS | (SEQ | VS | ID | (SEQ | |
| | NO: | (SEQ | ID | (SEQ | NO: | ID | |
| | 229) | ID | NO: | ID | 242) | NO: | |
| | | NO: | 235) | NO: | | 245) | |
| | | 232) | | 238) | | | |

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises a VHCDR1 amino acid sequence set forth in table 9;
VHCDR2 comprises a VHCDR2 amino acid sequence set forth in table 9;
VHCDR3 comprises a VHCDR3 amino acid sequence set forth in table 9;
VLCDR1 comprises a VLCDR1 amino acid sequence set forth in table 9;
VLCDR2 comprises a VLCDR2 amino acid sequence set forth in table 9; and
VLCDR3 comprises a VLCDR3 amino acid sequence set forth in table 9;
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 379 to 391 of SEQ ID NO: 1.

The specific binding molecule may bind to a polypeptide or protein molecule comprising an amino acid sequence comprising residues 379 to 391 of SEQ ID NO: 1 with a K_{D} of less than around 25 nM. The K_{D} may be less than around 20 nM, less than around 15 nM, or less than around 10 nM. The K_{D} may preferably be for binding to SEQ ID NO: 4. The specific binding molecule may have no detectable binding to SEQ ID NO: 1. The K_{D} for binding to SEQ ID NO: 4 may be around 300 pM to around 10 nM. The K_{D} for binding to SEQ ID NO: 4 may be around 300 pM to around 500 pM. The K_{D} for binding to SEQ ID NO: 4 may be around 1 nM to around 10 nM. The K_{D} for binding to SEQ ID NO: 4 may be around 401 pM, optionally wherein the specific binding molecule comprises the CDRs of E1E8. The K_{D} for binding to SEQ ID NO: 4 may be around 6.3 nM, optionally wherein the specific binding molecule comprises the CDRs of E1E8.

The specific binding molecule may comprise an amino acid sequence having at least 70%, at least 75%, at least 80%, at least 90%, at least 95% or at least 99% identity to SEQ ID NO: 248 wherein the specific binding molecule binds to a polypeptide or protein molecule comprising an amino acid sequence comprising residues 379 to 391 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 248. The CDRs may be 100% identical to the CDRs of SEQ ID NO: 248. The specific binding molecule may comprise an amino acid sequence having at least 70%, at least 75%, at least 80%, at least 90%, at least 95% or at least 99% identity to SEQ ID NO: 248 wherein CDRs are 100% identical to the CDRs of SEQ ID NO: 248. The specific binding molecule may comprise the amino acid sequence of SEQ ID NO: 248.

(E2E8 amino acid sequence)
SEQ ID NO: 248
QVQLQESGPSLVKPSQTLSLTCTVSGFSLTDRGVAWVRQAPGKAL
EWVGTMRSGGTIDYNPALKSRLSITRDTSKSQVFLSLSSVTTEDM
AMYYCARGYLSGDRYAWGRGLLVTVSSEGKSSGASGESKVDDQAV
LTQPSSVSKSLGQSVSIACSGSRSDIGYGNYVSWFQQIPGSAPKL
LIYDTNTRASGVPDRFSGARSGNTATLTINSLQAEDEADYYCANI
DSSRSHLFGSGTRLTVLG

The specific binding molecule may comprise an amino acid sequence having at least 70%, at least 75%, at least 80%, at least 90%, at least 95% or at least 99% identity to SEQ ID NO: 250 wherein the specific binding molecule binds to a polypeptide or protein molecule comprising an amino acid sequence comprising residues 379 to 391 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 250. The CDRs may be 100% identical to the CDRs of SEQ ID NO: 250. The specific binding molecule may comprise an amino acid sequence having at least 70%, at least 75%, at least 80%, at least 90%, at least 95% or at least 99% identity to SEQ ID NO: 250 wherein CDRs are 100% identical to the CDRs of SEQ ID NO: 250. The specific binding molecule may comprise the amino acid sequence of SEQ ID NO: 250.

(E1E8 amino acid sequence)
SEQ ID NO: 250
QVQLQESGPSLVKPSQTLSLTCTVSGFSLTDWGVAWVRQAPGKAL
EWLGTMRSGGTTDDNPALKSRLSITRDTSKSQVSLSLSSVTTEDM
AMYYCARGYLSGVHYAWGRGLLVTVSSEGKSSGASGESKVDDQAV
LTQPSSVSGSLGQSVSITCSGSSSNVGDGRYVSWFQQVPGSAPKL
LIYDTTSRASGVPDRFSGSRSGNTATLIITSLQAEDEADYYCASI
DSGNNLLFGSGTRLTVLG

The specific binding molecule may comprise an amino acid sequence having at least 70%, at least 75%, at least 80%, at least 90%, at least 95% or at least 99% identity to SEQ ID NO: 252 wherein the specific binding molecule binds to a polypeptide or protein molecule comprising an amino acid sequence comprising residues 379 to 391 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 252. The CDRs may be 100% identical to the CDRs of SEQ ID NO: 252. The specific binding molecule may comprise an amino acid sequence having at least 70%, at least 75%, at least 80%, at least 90%, at least 95% or at least 99% identity to SEQ ID NO: 252 wherein CDRs are 100% identical to the CDRs of SEQ ID NO: 252. The specific binding molecule may comprise the amino acid sequence of SEQ ID NO: 252.

(E2A6 amino acid sequence)
SEQ ID NO: 252
QVQLQESGPSLVKPSQTLSLTCTVSGFSLTDWGVAWVRQAPGKAL
EWLGTMRSGGTTDDNPALKSRLSITRDTSKSQVSLSLSSVTTEDM
AMYYCARGYLSGVHYAWGRGLLVTVSSEGKSSGASGESKVDDRVV
RTQPSSVSKSLGQSVSITCSGSSSNVGAGNYVGWFQQVPGSAPKL
LIYGATNRASGVPARFSGSKSGVTATLTITSLQAEDEADYYCASI
DTSRSHIFGSGTRLTVLG

The specific binding molecule may comprise an amino acid sequence having at least 70%, at least 75%, at least 80%, at least 90%, at least 95% or at least 99% identity to SEQ ID NO: 254 wherein the specific binding molecule binds to a polypeptide or protein molecule comprising an amino acid sequence comprising residues 379 to 391 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 254. The CDRs may be 100% identical to the CDRs of SEQ ID NO: 254. The specific binding molecule may comprise an amino acid sequence having at least 70%, at least 75%, at least 80%, at least 90%, at least 95% or at least 99% identity to SEQ ID NO: 254 wherein CDRs are 100% identical to the CDRs of SEQ ID NO: 254. The specific binding molecule may comprise the amino acid sequence of SEQ ID NO: 254.

(E2B7 amino acid sequence)
SEQ ID NO: 254
QVQLQESGPSLVKPSQTLSLTCTVSGFSLTSWGVAWVRQAPGKAL
EWLGTMRSGGGTEYNPALKSRLSITRDTSKSQVSLSLSSVTTEDM
AMYYCARGYLSGIHYAWGRGLLVSVSSEGKSSGASGESKVDDQAV
LTQLSSVSGSLGQRVSITCSGSSSNVGDGDYVGWFQQLPGSAPKL
LIYSARNRASGVPDRFSGSRSGNTATLTITSLQAEDEADYYCASI
DTSRSHIFGSGTRLTVLG

The epitope of the specific binding molecule may be within an amino acid sequence comprising residues 113 to 238 of SEQ ID NO: 1. Accordingly, the epitope may be within the amino acid sequence of SEQ ID NO: 255 (SLEDEAAGHVTQARMVSKSKDGTGSDDKKAKGADGKTKIATPRGAAPPGQKGQANATRIP AKTPPAPKTPPSSGEPPKSGDRSGYSSPGSPGTPGSRSRTPSLPTPPTREPKKVAVVRTP PKSPSS).

The epitope may be within an amino acid sequence comprising residues 113 to 238 of SEQ ID NO: 1. This epitope may be bound by the CDRs of the specific binding molecule referred to as “CB11” herein.

The epitope may comprise the amino acid sequence of SEQ ID NO: 255 (SLEDEAAGHVTQARMVSKSKDGTGSDDKKAKGADGKTKIATPRGAAPPGQKGQANATRIP AKTPPAPKTPPSSGEPPKSGDRSGYSSPGSPGTPGSRSRTPSLPTPPTREPKKVAVVRTP PKSPSS). The epitope may comprise an amino acid sequence having at least 70%, at least 75%, at least 80%, at least 90%, at least 95% or at least 99% identity to SEQ ID NO: 255 (SLEDEAAGHVTQARMVSKSKDGTGSDDKKAKGADGKTKIATPRGAAPPGQKGQANATRIP AKTPPAPKTPPSSGEPPKSGDRSGYSSPGSPGTPGSRSRTPSLPTPPTREPKKVAVVRTP PKSPSS).

The epitope may consist of the amino acid sequence of SEQ ID NO: 255 (SLEDEAAGHVTQARMVSKSKDGTGSDDKKAKGADGKTKIATPRGAAPPGQKGQANATRIP AKTPPAPKTPPSSGEPPKSGDRSGYSSPGSPGTPGSRSRTPSLPTPPTREPKKVAVVRTP PKSPSS). The epitope may consist of an amino acid sequence having at least 70%, at least 75%, at least 80%, at least 90%, at least 95% or at least 99% identity to SEQ ID NO: 255 (SLEDEAAGHVTQARMVSKSKDGTGSDDKKAKGADGKTKIATPRGAAPPGQKGQANATRIP AKTPPAPKTPPSSGEPPKSGDRSGYSSPGSPGTPGSRSRTPSLPTPPTREPKKVAVVRTP PKSPSS).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 198 (SNAVI);
VHCDR2 comprises the sequence set forth in SEQ ID NO: 200 (LIDVDGDAAYDPALKS);
VHCDR3 comprises the sequence set forth in SEQ ID NO: 202 (DYGSWGYVSDIDY);
VLCDR1 comprises the sequence set forth in SEQ ID NO: 256 (SGSNIGSNDVG);
VLCDR2 comprises the sequence set forth in SEQ ID NO: 257 (DNNNRPS); and
VLCDR3 comprises the sequence set forth in SEQ ID NO: 258 (GGYAGSSSNFL);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 113 to 238 of SEQ ID NO: 1. The specific binding molecule comprising CDRs having 100% identity to those given above is referred to as “CB11” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 198 (SNAVI);
VHCDR2 comprises the sequence set forth in SEQ ID NO: 200 (LIDVDGDAAYDPALKS);
VHCDR3 comprises the sequence set forth in SEQ ID NO: 202 (DYGSWGYVSDIDY);
VLCDR1 comprises the sequence set forth in SEQ ID NO: 256 (SGSNIGSNDVG);
VLCDR2 comprises the sequence set forth in SEQ ID NO: 257 (DNNNRPS); and
VLCDR3 comprises the sequence set forth in SEQ ID NO: 258 (GGYAGSSSNFL).

The epitope of the specific binding molecule may be within an amino acid sequence comprising residues 1 to 155 of SEQ ID NO: 1. Accordingly, the epitope may be within the amino acid sequence of SEQ ID NO: 293 (MAEPRQEFEVMEDHAGTYGLGDRKDQGGYTMHQDQEGDTDAGLKESPLQTPTEDGSEEPGSE TSDAKSTPTAEDVTAPLVDEGAPGKQAAAQPHTEIPEGTTAEEAGIGDTPSLEDEAAGHVTQARM VSKSKDGTGSDDKKAKGADGKTKIATPR).

The epitope may be within an amino acid sequence comprising residues 1 to 155 of SEQ ID NO: 1. This epitope may be bound by the CDRs of the specific binding molecule referred to as “CA2” herein.

The epitope may comprise the amino acid sequence of SEQ ID NO: 293 (MAEPRQEFEVMEDHAGTYGLGDRKDQGGYTMHQDQEGDTDAGLKESPLQTPTEDGSEEPGSE TSDAKSTPTAEDVTAPLVDEGAPGKQAAAQPHTEIPEGTTAEEAGIGDTPSLEDEAAGHVTQARM VSKSKDGTGSDDKKAKGADGKTKIATPR). The epitope may comprise an amino acid sequence having at least 70%, at least 75%, at least 80%, at least 90%, at least 95% or at least 99% identity to SEQ ID NO: 293 (MAEPRQEFEVMEDHAGTYGLGDRKDQGGYTMHQDQEGDTDAGLKESPLQTPTEDGSEEPGSE TSDAKSTPTAEDVTAPLVDEGAPGKQAAAQPHTEIPEGTTAEEAGIGDTPSLEDEAAGHVTQARM VSKSKDGTGSDDKKAKGADGKTKIATPR).

The epitope may consist of the amino acid sequence of SEQ ID NO: 293 (MAEPRQEFEVMEDHAGTYGLGDRKDQGGYTMHQDQEGDTDAGLKESPLQTPTEDGSEEPGSE TSDAKSTPTAEDVTAPLVDEGAPGKQAAAQPHTEIPEGTTAEEAGIGDTPSLEDEAAGHVTQARM VSKSKDGTGSDDKKAKGADGKTKIATPR). The epitope may consist of an amino acid sequence having at least 70%, at least 75%, at least 80%, at least 90%, at least 95% or at least 99% identity to SEQ ID NO: 293 (MAEPRQEFEVMEDHAGTYGLGDRKDQGGYTMHQDQEGDTDAGLKESPLQTPTEDGSEEPGSE TSDAKSTPTAEDVTAPLVDEGAPGKQAAAQPHTEIPEGTTAEEAGIGDTPSLEDEAAGHVTQARM VSKSKDGTGSDDKKAKGADGKTKIATPR).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 259 (SNGVG);
VHCDR2 comprises the sequence set forth in SEQ ID NO: 157 (DISSVGKKYANPALKS);
VHCDR3 comprises the sequence set forth in SEQ ID NO: 161 (CRDGGVTYGYDIDY);
VLCDR1 comprises the sequence set forth in SEQ ID NO: 165 (SGSSGNVGYGDYVS);
VLCDR2 comprises the sequence set forth in SEQ ID NO: 169 (GATNLAS); and
VLCDR3 comprises the sequence set forth in SEQ ID NO: 173 (ASYDSSSGGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 1 to 155 of SEQ ID NO: 1. The specific binding molecule comprising CDRs having 100% identity to those given above is referred to as “CA2” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 259 (SNGVG);
VHCDR2 comprises the sequence set forth in SEQ ID NO: 157 (DISSVGKKYANPALKS);
VHCDR3 comprises the sequence set forth in SEQ ID NO: 161 (CRDGGVTYGYDIDY);
VLCDR1 comprises the sequence set forth in SEQ ID NO: 165 (SGSSGNVGYGDYVS);
VLCDR2 comprises the sequence set forth in SEQ ID NO: 169 (GATNLAS); and
VLCDR3 comprises the sequence set forth in SEQ ID NO: 173 (ASYDSSSGGV).

The epitope of the specific binding molecule may be within an amino acid sequence comprising residues 1 to 238 of SEQ ID NO: 1. Accordingly, the epitope may be within the amino acid sequence of SEQ ID NO: 260 (MAEPRQEFEVMEDHAGTYGLGDRKDQGGYTMHQDQEGDTDAGLKESPLQTPTEDGSEEPGSE TSDAKSTPTAEDVTAPLVDEGAPGKQAAAQPHTEIPEGTTAEEAGIGDTPSLEDEAAGHVTQARM VSKSKDGTGSDDKKAKGADGKTKIATPRGAAPPGQKGQANATRIPAKTPPAPKTPPSSGEPPKSG DRSGYSSPGSPGTPGSRSRTPSLPTPPTREPKKVAVVRTPPKSPSS).

The epitope may be within an amino acid sequence comprising residues 1 to 238 of SEQ ID NO: 1. This epitope may be bound by the CDRs of the specific binding molecule referred to as “CB6” herein.

The epitope may comprise the amino acid sequence of SEQ ID NO: 260 (MAEPRQEFEVMEDHAGTYGLGDRKDQGGYTMHQDQEGDTDAGLKESPLQTPTEDGSEEPGSE TSDAKSTPTAEDVTAPLVDEGAPGKQAAAQPHTEIPEGTTAEEAGIGDTPSLEDEAAGHVTQARM VSKSKDGTGSDDKKAKGADGKTKIATPRGAAPPGQKGQANATRIPAKTPPAPKTPPSSGEPPKSG DRSGYSSPGSPGTPGSRSRTPSLPTPPTREPKKVAVVRTPPKSPSS). The epitope may comprise an amino acid sequence having at least 70%, at least 75%, at least 80%, at least 90%, at least 95% or at least 99% to SEQ identity ID NO: 260 (MAEPRQEFEVMEDHAGTYGLGDRKDQGGYTMHQDQEGDTDAGLKESPLQTPTEDGSEEPGSE TSDAKSTPTAEDVTAPLVDEGAPGKQAAAQPHTEIPEGTTAEEAGIGDTPSLEDEAAGHVTQARM VSKSKDGTGSDDKKAKGADGKTKIATPRGAAPPGQKGQANATRIPAKTPPAPKTPPSSGEPPKSG DRSGYSSPGSPGTPGSRSRTPSLPTPPTREPKKVAVVRTPPKSPSS).

The epitope may consist of the amino acid sequence of SEQ ID NO: 260 (MAEPRQEFEVMEDHAGTYGLGDRKDQGGYTMHQDQEGDTDAGLKESPLQTPTEDGSEEPGSE TSDAKSTPTAEDVTAPLVDEGAPGKQAAAQPHTEIPEGTTAEEAGIGDTPSLEDEAAGHVTQARM VSKSKDGTGSDDKKAKGADGKTKIATPRGAAPPGQKGQANATRIPAKTPPAPKTPPSSGEPPKSG DRSGYSSPGSPGTPGSRSRTPSLPTPPTREPKKVAVVRTPPKSPSS). The epitope may consist of an amino acid sequence having at least 70%, at least 75%, at least 80%, at least 90%, at least 95% or at least 99% identity to SEQ ID NO: 260 (MAEPRQEFEVMEDHAGTYGLGDRKDQGGYTMHQDQEGDTDAGLKESPLQTPTEDGSEEPGSE TSDAKSTPTAEDVTAPLVDEGAPGKQAAAQPHTEIPEGTTAEEAGIGDTPSLEDEAAGHVTQARM VSKSKDGTGSDDKKAKGADGKTKIATPRGAAPPGQKGQANATRIPAKTPPAPKTPPSSGEPPKSG DRSGYSSPGSPGTPGSRSRTPSLPTPPTREPKKVAVVRTPPKSPSS).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 259 (SNGVG);
VHCDR2 comprises the sequence set forth in SEQ ID NO: 157 (DISSVGKKYANPALKS);
VHCDR3 comprises the sequence set forth in SEQ ID NO: 161 (CRDGGVTYGYDIDY);
VLCDR1 comprises the sequence set forth in SEQ ID NO: 261 (SGSSSNIGTGNYVG);
VLCDR2 comprises the sequence set forth in SEQ ID NO: 262 (GAVTRAS); and
VLCDR3 comprises the sequence set forth in SEQ ID NO: 263 (ASYDSTSGGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 1 to 238 of SEQ ID NO: 1. The specific binding molecule comprising CDRs having 100% identity to those given above is referred to as “CB6” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 259 (SNGVG);
VHCDR2 comprises the sequence set forth in SEQ ID NO: 157 (DISSVGKKYANPALKS);
VHCDR3 comprises the sequence set forth in SEQ ID NO: 161 (CRDGGVTYGYDIDY);
VLCDR1 comprises the sequence set forth in SEQ ID NO: 261 (SGSSSNIGTGNYVG);
VLCDR2 comprises the sequence set forth in SEQ ID NO: 262 (GAVTRAS); and
VLCDR3 comprises the sequence set forth in SEQ ID NO: 263 (ASYDSTSGGV).

The epitope of the specific binding molecule may be within an amino acid sequence comprising residues 1 to 319 of SEQ ID NO: 1. Accordingly, the epitope may be within the amino acid sequence of SEQ ID NO: 264 (MAEPRQEFEVMEDHAGTYGLGDRKDQGGYTMHQDQEGDTDAGLKESPLQTPTEDGSEEPGSE TSDAKSTPTAEDVTAPLVDEGAPGKQAAAQPHTEIPEGTTAEEAGIGDTPSLEDEAAGHVTQARM VSKSKDGTGSDDKKAKGADGKTKIATPRGAAPPGQKGQANATRIPAKTPPAPKTPPSSGEPPKSG DRSGYSSPGSPGTPGSRSRTPSLPTPPTREPKKVAVVRTPPKSPSSAKSRLQTAPVPMPDLKNVK SKIGSTENLKHQPGGGKVQIINKKLDLSNVQSKCGSKDNIKHVPGGGSVQIVYKPVDLSKVT).

The epitope may be within an amino acid sequence comprising residues 1 to 319 of SEQ ID NO: 1. This epitope may be bound by the CDRs of the specific binding molecules referred to as “CA7”, “CA8”, and “CB10” herein.

The epitope within an amino acid sequence comprising residues 1 to 319 of SEQ ID NO: 1 may preferably be within an amino acid sequence comprising residues 37 to 49 of SEQ ID NO: 1. This epitope may be bound by the CDRs of at least the specific binding molecule referred to as “CA7” herein.

The epitope may comprise the amino acid sequence of SEQ ID NO: 264 (MAEPRQEFEVMEDHAGTYGLGDRKDQGGYTMHQDQEGDTDAGLKESPLQTPTEDGSEEPGSE TSDAKSTPTAEDVTAPLVDEGAPGKQAAAQPHTEIPEGTTAEEAGIGDTPSLEDEAAGHVTQARM VSKSKDGTGSDDKKAKGADGKTKIATPRGAAPPGQKGQANATRIPAKTPPAPKTPPSSGEPPKSG DRSGYSSPGSPGTPGSRSRTPSLPTPPTREPKKVAVVRTPPKSPSSAKSRLQTAPVPMPDLKNVK SKIGSTENLKHQPGGGKVQIINKKLDLSNVQSKCGSKDNIKHVPGGGSVQIVYKPVDLSKVT). The epitope may comprise an amino acid sequence having at least 70%, at least 75%, at least 80%, at least 90%, at least 95% at or least identity SEQ ID 99% to NO: 264 (MAEPRQEFEVMEDHAGTYGLGDRKDQGGYTMHQDQEGDTDAGLKESPLQTPTEDGSEEPGSE TSDAKSTPTAEDVTAPLVDEGAPGKQAAAQPHTEIPEGTTAEEAGIGDTPSLEDEAAGHVTQARM VSKSKDGTGSDDKKAKGADGKTKIATPRGAAPPGQKGQANATRIPAKTPPAPKTPPSSGEPPKSG DRSGYSSPGSPGTPGSRSRTPSLPTPPTREPKKVAVVRTPPKSPSSAKSRLQTAPVPMPDLKNVK SKIGSTENLKHQPGGGKVQIINKKLDLSNVQSKCGSKDNIKHVPGGGSVQIVYKPVDLSKVT).

The epitope may consist of the amino acid sequence of SEQ ID NO: 264 (MAEPRQEFEVMEDHAGTYGLGDRKDQGGYTMHQDQEGDTDAGLKESPLQTPTEDGSEEPGSE TSDAKSTPTAEDVTAPLVDEGAPGKQAAAQPHTEIPEGTTAEEAGIGDTPSLEDEAAGHVTQARM VSKSKDGTGSDDKKAKGADGKTKIATPRGAAPPGQKGQANATRIPAKTPPAPKTPPSSGEPPKSG DRSGYSSPGSPGTPGSRSRTPSLPTPPTREPKKVAVVRTPPKSPSSAKSRLQTAPVPMPDLKNVK SKIGSTENLKHQPGGGKVQIINKKLDLSNVQSKCGSKDNIKHV PGGGSVQIVYKPVDLSKVT). The epitope may consist of an amino acid sequence having at least 70%, at least 75%, at least 80%, at least 90%, at least 95% or at least 99% identity to SEQ ID NO: 264 (MAEPRQEFEVMEDHAGTYGLGDRKDQGGYTMHQDQEGDTDAGLKESPLQTPTEDGSEEPGSE TSDAKSTPTAEDVTAPLVDEGAPGKQAAAQPHTEIPEGTTAEEAGIGDTPSLEDEAAGHVTQARM VSKSKDGTGSDDKKAKGADGKTKIATPRGAAPPGQKGQANATRIPAKTPPAPKTPPSSGEPPKSG DRSGYSSPGSPGTPGSRSRTPSLPTPPTREPKKVAVVRTPPKSPSSAKSRLQTAPVPMPDLKNVK SKIGSTENLKHQPGGGKVQIINKKLDLSNVQSKCGSKDNIKHVPGGGSVQIVYKPVDLSKVT).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 44 (SYYVG);
VHCDR2 comprises the sequence set forth in SEQ ID NO: 52 (NIYSTGRAFYNPALKS);
VHCDR3 comprises the sequence set forth in SEQ ID NO: 265 (GSYYHGGGNGMVDFFDY);
VLCDR1 comprises the sequence set forth in SEQ ID NO: 39 (SGSSSNVGYGNYVG);
VLCDR2 comprises the sequence set forth in SEQ ID NO: 72 (AATSRAS); and
VLCDR3 comprises the sequence set forth in SEQ ID NO: 78 (SSYQRGNTGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 1 to 319 of SEQ ID NO: 1. The specific binding molecule comprising CDRs having 100% identity to those given above is referred to as “CA7” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 44 (SYYVG);
VHCDR2 comprises the sequence set forth in SEQ ID NO: 52 (NIYSTGRAFYNPALKS);
VHCDR3 comprises the sequence set forth in SEQ ID NO: 265 (GSYYHGGGNGMVDFFDY);
VLCDR1 comprises the sequence set forth in SEQ ID NO: 39 (SGSSSNVGYGNYVG);
VLCDR2 comprises the sequence set forth in SEQ ID NO: 72 (AATSRAS); and
VLCDR3 comprises the sequence set forth in SEQ ID NO: 78 (SSYQRGNTGV).

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 495 (RVRLQGSGPSLVKPSQTLSLTCTVSGFSFD);
VHFR2 comprises the sequence set forth in SEQ ID NO: 496 (WVRQAPGKALEWLG);
VHFR3 comprises the sequence set forth in SEQ ID NO: 497 (RLSITRDTSKSQVSLSVSSVTIEDTALYYCVR);
VHFR4 comprises the sequence set forth in SEQ ID NO: 498 (WSPGLLVTVSS);
VLFR1 comprises the sequence set forth in SEQ ID NO: 499 (QVVRTQPSSVSGSLGQRVSITC);
VLFR2 comprises the sequence set forth in SEQ ID NO: 500 (WFQQVPGSAPKLLIY);
VLFR3 comprises the sequence set forth in SEQ ID NO: 501 (GVPDRFSGSRSGNTATLTIDSLQAEDEADYYC);
VLFR4 comprises the sequence set forth in SEQ ID NO: 502 (FGSGTRLTVLG);
or for each FR sequence, an amino acid sequence with
   (i) at least 50% identity thereto, and/or
   (ii) one, two, three, four or five amino acid substitutions relative thereto.

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 495 (RVRLQGSGPSLVKPSQTLSLTCTVSGFSFD);
VHFR2 comprises the sequence set forth in SEQ ID NO: 496 (WVRQAPGKALEWLG);
VHFR3 comprises the sequence set forth in SEQ ID NO: 497 (RLSITRDTSKSQVSLSVSSVTIEDTALYYCVR);
VHFR4 comprises the sequence set forth in SEQ ID NO: 498 (WSPGLLVTVSS);
VLFR1 comprises the sequence set forth in SEQ ID NO: 499 (QVVRTQPSSVSGSLGQRVSITC);
VLFR2 comprises the sequence set forth in SEQ ID NO: 500 (WFQQVPGSAPKLLIY);
VLFR3 comprises the sequence set forth in SEQ ID NO: 501 (GVPDRFSGSRSGNTATLTIDSLQAEDEADYYC);
VLFR4 comprises the sequence set forth in SEQ ID NO: 502 (FGSGTRLTVLG);
or for each FR sequence, an amino acid sequence with
   (i) at least 50% identity thereto, and/or
   (ii) one, two, three, four or five amino acid substitutions relative thereto,
wherein the specific binding molecule binds to a polypeptide or protein molecule comprising an amino acid sequence comprising residues 1 to 319 of SEQ ID NO: 1. A specific binding molecule comprising FRs having 100% identity to those given above is referred to as “CA7” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 495 (RVRLQGSGPSLVKPSQTLSLTCTVSGFSFD);
   VHFR2 comprises the sequence set forth in SEQ ID NO: 496 (WVRQAPGKALEWLG);
   VHFR3 comprises the sequence set forth in SEQ ID NO: 497 (RLSITRDTSKSQVSLSVSSVTIEDTALYYCVR);
   VHFR4 comprises the sequence set forth in SEQ ID NO: 498 (WSPGLLVTVSS);
   VLFR1 comprises the sequence set forth in SEQ ID NO: 499 (QVVRTQPSSVSGSLGQRVSITC);
   VLFR2 comprises the sequence set forth in SEQ ID NO: 500 (WFQQVPGSAPKLLIY);
   VLFR3 comprises the sequence set forth in SEQ ID NO: 501 (GVPDRFSGSRSGNTATLTIDSLQAEDEADYYC);
   VLFR4 comprises the sequence set forth in SEQ ID NO: 502 (FGSGTRLTVLG);
   or for each FR sequence, an amino acid sequence with
   (i) at least 50% identity thereto, and/or
   (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 44 (SYYVG);
   VHCDR2 comprises the sequence set forth in SEQ ID NO: 52 (NIYSTGRAFYNPALKS);
   VHCDR3 comprises the sequence set forth in SEQ ID NO: 265 (GSYYHGGGNGMVDFFDY);
   VLCDR1 comprises the sequence set forth in SEQ ID NO: 39 (SGSSSNVGYGNYVG);
   VLCDR2 comprises the sequence set forth in SEQ ID NO: 72 (AATSRAS); and
   VLCDR3 comprises the sequence set forth in SEQ ID NO: 78 (SSYQRGNTGV).
   or for each CDR sequence, an amino acid sequence with
      (i) at least 85% identity thereto, and/or
      (ii) one, two, or three amino acid substitutions relative thereto
   wherein the specific binding molecule binds to a polypeptide or protein molecule comprising an amino acid sequence comprising residues 1 to 319 of SEQ ID NO: 1. The specific binding molecule comprising FRs and CDRs having 100% identity to those given above is referred to as “CA7” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 495 (RVRLQGSGPSLVKPSQTLSLTCTVSGFSFD);
   VHFR2 comprises the sequence set forth in SEQ ID NO: 496 (WVRQAPGKALEWLG);
   VHFR3 comprises the sequence set forth in SEQ ID NO: 497 (RLSITRDTSKSQVSLSVSSVTIEDTALYYCVR);
   VHFR4 comprises the sequence set forth in SEQ ID NO: 498 (WSPGLLVTVSS);
   VLFR1 comprises the sequence set forth in SEQ ID NO: 499 (QVVRTQPSSVSGSLGQRVSITC);
   VLFR2 comprises the sequence set forth in SEQ ID NO: 500 (WFQQVPGSAPKLLIY);
   VLFR3 comprises the sequence set forth in SEQ NO: 501 (GVPDRFSGSRSGNTATLTIDSLQAEDEADYYC);
   VLFR4 comprises the sequence set forth in SEQ ID NO: 502 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 44 (SYYVG);
   VHCDR2 comprises the sequence set forth in SEQ ID NO: 52 (NIYSTGRAFYNPALKS);
   VHCDR3 comprises sequence set forth in SEQ ID NO: 265 (GSYYHGGGNGMVDFFDY);
   VLCDR1 comprises the sequence set forth in SEQ ID NO: 39 (SGSSSNVGYGNYVG);
   VLCDR2 comprises the sequence set forth in SEQ ID NO: 72 (AATSRAS); and
   VLCDR3 comprises the sequence set forth in SEQ ID NO: 78 (SSYQRGNTGV).
wherein the specific binding molecule binds to a polypeptide or protein molecule comprising an amino acid sequence comprising residues 1 to 319 of SEQ ID NO: 1. The specific binding molecule comprising FRs and CDRs having 100% identity to those given above is referred to as “CA7” herein.

The specific binding molecule may comprise:
(a) A VH domain comprising the sequence set forth in SEQ ID NO: 503 (RVRLQGSGPSLVKPSQTLSLTCTVSGFSFDSYYVGWVRQAPGKALEWLGNIYSTGRAFY NPALKSRLSITRDTSKSQVSLSVSSVTIEDTALYYCVRGSYYHGGGNGMVDFFDYWSPGL LVTVSS); and/or
(b) a VL domain comprising the sequence set forth in SEQ ID NO: 504 (QVVRTQPSSVSGSLGQRVSITCSGSSSNVGYGNYVGWFQQVPGSAPKLLIYAATSRAS GVPDRFSGSRSGNTATLTIDSLQAEDEADYYCSSYQRGNTGVFGSGTRLTVLG);
or a humanized variant thereof.

The specific binding molecule may comprise:
(a) A heavy chain comprising the sequence set forth in SEQ ID NO: 505 (RVRLQGSGPSLVKPSQTLSLTCTVSGFSFDSYYVGWVRQAPGKALEWLGNIYSTGRAFY NPALKSRLSITRDTSKSQVSLSVSSVTIEDTALYYCVRGSYYHGGGNGMVDFFDYWSPGL LVTVSSAKTTAPSVYPLAPVCGDTTGSSVTLGCLVKGYFPEPVTLTWNSGSLSSGVHTFP AVLQSDLYTLSSSVTVTSSTWPSQSITCNVAHPASSTKVDKKIEPRGPTIKPCPPCKCPAP NLLGGPSVFIFPPKIKDVLMISLSPIVTCVVVDVSEDDPDVQISWFVNNVEVHTAQTQTHRE DYNSTLRVVSALPIQHQDWMSGKEFKCKVNNKDLPAPIERTISKPKGSVRAPQVYVLPPP EEEMTKKQVTLTCMVTDFMPEDIYVEWTNNGKTELNYKNTEPVLDSDGSYFMYSKLRVE KKNWVERNSYSCSVVHEGLHNHHTTKSFSRTPGK); and/or
(b) a light chain comprising the sequence set forth in SEQ ID NO: 506 (QVVRTQPSSVSGSLGQRVSITCSGSSSNVGYGNYVGWFQQVPGSAPKLLIYAATSRAS GVPDRFSGSRSGNTATLTIDSLQAEDEADYYCSSYQRGNTGVFGSGTRLTVLGGQPKSS PSVTLFPPSSEELETNKATLVCTITDFYPGVVTVDWKVDGTPVTQGMETTQPSKQSNNKY MASSYLTLTARAWERHSSYSCQVTHEGHTVEKSLSRADCS);
or a humanized variant thereof.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 266 (SNAVV);
VHCDR2 comprises the sequence set forth in SEQ ID NO: 267 (AIDKDGDTIYNPALKS);
VHCDR3 comprises the sequence set forth in SEQ ID NO: 268 (DPSGWGYPDVDY);
VLCDR1 comprises the sequence set forth in SEQ ID NO: 269 (SGTYIGSSDVG);
VLCDR2 comprises the sequence set forth in SEQ ID NO: 270 (GTSSRPS); and
VLCDR3 comprises the sequence set forth in SEQ ID NO: 271 (ATYESSYHNSV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 1 to 319 of SEQ ID NO: 1. The specific binding molecule comprising CDRs having 100% identity to those given above is referred to as “CA8” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 266 (SNAVV);
VHCDR2 comprises the sequence set forth in SEQ ID NO: 267 (AIDKDGDTIYNPALKS);
VHCDR3 comprises the sequence set forth in SEQ ID NO: 268 (DPSGWGYPDVDY);
VLCDR1 comprises the sequence set forth in SEQ ID NO: 269 (SGTYIGSSDVG);
VLCDR2 comprises the sequence set forth in SEQ ID NO: 270 (GTSSRPS); and
VLCDR3 comprises the sequence set forth in SEQ ID NO: 271 (ATYESSYHNSV).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 272 (SNTVA);
VHCDR2 comprises the sequence set forth in SEQ ID NO: 273 (EINSGGSTYYNPALKS);
VHCDR3 comprises the sequence set forth in SEQ ID NO: 274 (GARSTYAAY);
VLCDR1 comprises the sequence set forth in SEQ ID NO: 275 (SGSSSDVGYSTWVY);
VLCDR2 comprises the sequence set forth in SEQ ID NO: 276 (HISNRAS); and
VLCDR3 comprises the sequence set forth in SEQ ID NO: 277 (AAYDSSNNVWI);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 1 to 319 of SEQ ID NO: 1. The specific binding molecule comprising CDRs having 100% identity to those given above is referred to as “CB10” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 272 (SNTVA);
VHCDR2 comprises the sequence set forth in SEQ ID NO: 273 (EINSGGSTYYNPALKS);
VHCDR3 comprises the sequence set forth in SEQ ID NO: 274 (GARSTYAAY);
VLCDR1 comprises the sequence set forth in SEQ ID NO: 275 (SGSSSDVGYSTWVY);
VLCDR2 comprises the sequence set forth in SEQ ID NO: 276 (HISNRAS); and
VLCDR3 comprises the sequence set forth in SEQ ID NO: 277 (AAYDSSNNVWI).

The epitope of the specific binding molecule may be within an amino acid sequence comprising residues 13 to 25 of SEQ ID NO: 1. Accordingly, the epitope may be within the amino acid sequence of SEQ ID NO: 278 (DHAGTYGLGDRKD).

The epitope may be within an amino acid sequence comprising residues 13 to 25 of SEQ ID NO: 1. This epitope may be bound by the CDRs of the specific binding molecule referred to as “CB7” herein.

The epitope may comprise the amino acid sequence of SEQ ID NO: 278 (DHAGTYGLGDRKD). The epitope may comprise an amino acid sequence having at least 70%, at least 75%, at least 80%, at least 90%, at least 95% or at least 99% identity to SEQ ID NO: 278 (DHAGTYGLGDRKD).

The epitope may consist of the amino acid sequence of SEQ ID NO: 278 (DHAGTYGLGDRKD). The epitope may consist of an amino acid sequence having at least 70%, at least 75%, at least 80%, at least 90%, at least 95% or at least 99% identity to SEQ ID NO: 278 (DHAGTYGLGDRKD).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 279 (NYRVG);
VHCDR2 comprises the sequence set forth in SEQ ID NO: 280 (NIRSGGTTWYNPALKS);
VHCDR3 comprises the sequence set forth in SEQ ID NO: 281 (DSSGDLYAYDY);
VLCDR1 comprises the sequence set forth in SEQ ID NO: 282 (SGSSSNVGYGNYMA);
VLCDR2 comprises the sequence set forth in SEQ ID NO: 141 (GATSRAS); and
VLCDR3 comprises the sequence set forth in SEQ ID NO: 263 (ASYDSTSGGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 13 to 25 of SEQ ID NO: 1. The specific binding molecule comprising CDRs having 100% identity to those given above is referred to as “CB7” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 279 (NYRVG);
VHCDR2 comprises the sequence set forth in SEQ ID NO: 280 (NIRSGGTTWYNPALKS);
VHCDR3 comprises the sequence set forth in SEQ ID NO: 281 (DSSGDLYAYDY);
VLCDR1 comprises the sequence set forth in SEQ ID NO: 282 (SGSSSNVGYGNYMA);
VLCDR2 comprises the sequence set forth in SEQ ID NO: 141 (GATSRAS); and
VLCDR3 comprises the sequence set forth in SEQ ID NO: 263 (ASYDSTSGGV).

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 507 (QVQLQESGPSLVKPSQTLSLTCTVSGFSLT);
VHFR2 comprises the sequence set forth in SEQ ID NO: 508 (WVRQAPGKALEWVS);
VHFR3 comprises the sequence set forth in SEQ ID NO: 509 (RLSITADTSKSQVSLSLSSVTTEDTAVYYCAR);
VHFR4 comprises the sequence set forth in SEQ ID NO: 510 (WGPGLLVTVSS);
VLFR1 comprises the sequence set forth in SEQ ID NO: 511 (QAVLTQPSSVSRSLGQSVSMTC);
VLFR2 comprises the sequence set forth in SEQ ID NO: 512 (WFQQVPGSAPKLLIY);
VLFR3 comprises the sequence set forth in SEQ ID NO: 513 (GVPDRFSGSRSGNTATLTISSLQAEDEADYYC);
VLFR4 comprises the sequence set forth in SEQ ID NO:514 (FGSGTRLTVLG);
or for each FR sequence, an amino acid sequence with
   (i) at least 50% identity thereto, and/or
   (ii) one, two, three, four or five amino acid substitutions relative thereto.

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 507 (QVQLQESGPSLVKPSQTLSLTCTVSGFSLT);
VHFR2 comprises the sequence set forth in SEQ ID NO: 508 (WVRQAPGKALEWVS);
VHFR3 comprises the sequence set forth in SEQ ID NO: 509 (RLSITADTSKSQVSLSLSSVTTEDTAVYYCAR);
VHFR4 comprises the sequence set forth in SEQ ID NO: 510 (WGPGLLVTVSS);
VLFR1 comprises the sequence set forth in SEQ ID NO: 511 (QAVLTQPSSVSRSLGQSVSMTC);
VLFR2 comprises the sequence set forth in SEQ ID NO: 512 (WFQQVPGSAPKLLIY);
VLFR3 comprises the sequence set forth in SEQ ID NO: 513 (GVPDRFSGSRSGNTATLTISSLQAEDEADYYC);
VLFR4 comprises the sequence set forth in SEQ ID NO: 514 (FGSGTRLTVLG);
or for each FR sequence, an amino acid sequence with
   (i) at least 50% identity thereto, and/or
   (ii) one, two, three, four or five amino acid substitutions relative thereto,
wherein the specific binding molecule binds to a polypeptide or protein molecule comprising an amino acid sequence comprising residues 13 to 25 of SEQ ID NO: 1. A specific binding molecule comprising FRs having 100% identity to those given above is referred to as “CB7” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 507 (QVQLQESGPSLVKPSQTLSLTCTVSGFSLT);
   VHFR2 comprises the sequence set forth in SEQ ID NO: 508 (WVRQAPGKALEWVS);
   VHFR3 comprises the sequence set forth in SEQ ID NO: 509 (RLSITADTSKSQVSLSLSSVTTEDTAVYYCAR);
   VHFR4 comprises the sequence set forth in SEQ ID NO: 510 (WGPGLLVTVSS);
   VLFR1 comprises the sequence set forth in SEQ ID NO: 511 (QAVLTQPSSVSRSLGQSVSMTC);
   VLFR2 comprises the sequence set forth in SEQ ID NO: 512 (WFQQVPGSAPKLLIY);
   VLFR3 comprises the sequence set forth in SEQ ID NO: 513 (GVPDRFSGSRSGNTATLTISSLQAEDEADYYC);
   VLFR4 comprises the sequence set forth in SEQ ID NO: 514 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 279 (NYRVG);
   VHCDR2 comprises the sequence set forth in SEQ ID NO: 280 (NIRSGGTTWYNPALKS);
   VHCDR3 comprises the sequence set forth in SEQ ID NO: 281 (DSSGDLYAYDY);
   VLCDR1 comprises the sequence set forth in SEQ ID NO: 282 (SGSSSNVGYGNYMA);
   VLCDR2 comprises the sequence set forth in SEQ ID NO: 141 (GATSRAS); and
   VLCDR3 comprises the sequence set forth in SEQ ID NO: 263 (ASYDSTSGGV);
   or for each CDR sequence, an amino acid sequence with
      (i) at least 85% identity thereto, and/or
      (ii) one, two, or three amino acid substitutions relative thereto
   wherein the specific binding molecule binds to a polypeptide or protein molecule comprising an amino acid sequence comprising residues 13 to 25 of SEQ ID NO: 1. The specific binding molecule comprising FRs and CDRs having 100% identity to those given above is referred to as “CB7” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 507 (QVQLQESGPSLVKPSQTLSLTCTVSGFSLT);
   VHFR2 comprises the sequence set forth in SEQ ID NO: 508 (WVRQAPGKALEWVS);
   VHFR3 comprises the sequence set forth in SEQ ID NO: 509 (RLSITADTSKSQVSLSLSSVTTEDTAVYYCAR);
   VHFR4 comprises the sequence set forth in SEQ ID NO: 510 (WGPGLLVTVSS);
   VLFR1 comprises the sequence set forth in SEQ ID NO: 511 (QAVLTQPSSVSRSLGQSVSMTC);
   VLFR2 comprises the sequence set forth in SEQ ID NO: 512 (WFQQVPGSAPKLLIY);
   VLFR3 comprises the sequence set forth in SEQ ID NO: 513 (GVPDRFSGSRSGNTATLTISSLQAEDEADYYC);
   VLFR4 comprises the sequence set forth in SEQ ID NO: 514 (FGSGTRLTVLG);
   or for each FR sequence, an amino acid sequence with
   (i) at least 50% identity thereto, and/or
   (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 279 (NYRVG);
   VHCDR2 comprises the sequence set forth in SEQ ID NO: 280 (NIRSGGTTWYNPALKS);
   VHCDR3 comprises the sequence set forth in SEQ ID NO: 281 (DSSGDLYAYDY);
   VLCDR1 comprises the sequence set forth in SEQ ID NO: 282 (SGSSSNVGYGNYMA);
   VLCDR2 comprises the sequence set forth in SEQ ID NO: 141 (GATSRAS); and
   VLCDR3 comprises the sequence set forth in SEQ ID NO: 263 (ASYDSTSGGV);
   wherein the specific binding molecule binds to a polypeptide or protein molecule comprising an amino acid sequence comprising residues 13 to 25 of SEQ ID NO: 1. The specific binding molecule comprising FRs and CDRs having 100% identity to those given above is referred to as “CB7” herein.

The specific binding molecule may comprise:
(a) A VH domain comprising the sequence set forth in SEQ ID NO: 515 (QVQLQESGPSLVKPSQTLSLTCTVSGFSLTNYRVGWVRQAPGKALEWVSNIRSGGTTW YNPALKSRLSITADTSKSQVSLSLSSVTTEDTAVYYCARDSSGDLYAYDYWGPGLLVTVS S); and/or
(b) a VL domain comprising the sequence set forth in SEQ ID NO: 516 (QAVLTQPSSVSRSLGQSVSMTCSGSSSNVGYGNYMAWFQQVPGSAPKLLIYGATSRAS GVPDRFSGSRSGNTATLTISSLQAEDEADYYCASYDSTSGGVFGSGTRLTVLG);
or a humanized variant thereof.

The specific binding molecule may comprise:
(a) A heavy chain comprising the sequence set forth in SEQ ID NO: 517 (QVQLQESGPSLVKPSQTLSLTCTVSGFSLTNYRVGWVRQAPGKALEWVSNIRSGGTTW YNPALKSRLSITADTSKSQVSLSLSSVTTEDTAVYYCARDSSGDLYAYDYWGPGLLVTVS SAKTTAPSVYPLAPVCGDTTGSSVTLGCLVKGYFPEPVTLTWNSGSLSSGVHTFPAVLQS DLYTLSSSVTVTSSTWPSQSITCNVAHPASSTKVDKKIEPRGPTIKPCPPCKCPAPNLLGG PSVFIFPPKIKDVLMISLSPIVTCVVVDVSEDDPDVQISWFVNNVEVHTAQTQTHREDYNST LRVVSALPIQHQDWMSGKEFKCKVNNKDLPAPIERTISKPKGSVRAPQVYVLPPPEEEMT KKQVTLTCMVTDFMPEDIYVEWTNNGKTELNYKNTEPVLDSDGSYFMYSKLRVEKKNWV ERNSYSCSVVHEGLHNHHTTKSFSRTPGK); and/or
(b) a light chain comprising the sequence set forth in SEQ ID NO: 518 (QAVLTQPSSVSRSLGQSVSMTCSGSSSNVGYGNYMAWFQQVPGSAPKLLIYGATSRAS GVPDRFSGSRSGNTATLTISSLQAEDEADYYCASYDSTSGGVFGSGTRLTVLGGQPKSS PSVTLFPPSSEELETNKATLVCTITDFYPGVVTVDWKVDGTPVTQGMETTQPSKQSNNKY MASSYLTLTARAWERHSSYSCQVTHEGHTVEKSLSRADCS);
or a humanized variant thereof.

The epitope of the specific binding molecule may be within an amino acid sequence comprising residues 145 to 157 of SEQ ID NO: 1. Accordingly, the epitope may be within the amino acid sequence of SEQ ID NO: 283 (ADGKTKIATPRGA).

The epitope may be within an amino acid sequence comprising residues 145 to 157 of SEQ ID NO: 1. This epitope may be bound by the CDRs of the specific binding molecule referred to as “CC7” herein.

The epitope may comprise the amino acid sequence of SEQ ID NO: 283 (ADGKTKIATPRGA). The epitope may comprise an amino acid sequence having at least 70%, at least 75%, at least 80%, at least 90%, at least 95% or at least 99% identity to SEQ ID NO: 283 (ADGKTKIATPRGA).

The epitope may consist of the amino acid sequence of SEQ ID NO: 283 (ADGKTKIATPRGA). The epitope may consist of an amino acid sequence having at least 70%, at least 75%, at least 80%, at least 90%, at least 95% or at least 99% identity to SEQ ID NO: 283 (ADGKTKIATPRGA).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 198 (SNAVI);
VHCDR2 comprises the sequence set forth in SEQ ID NO: 200 (LIDVDGDAAYDPALKS);
VHCDR3 comprises the sequence set forth in SEQ ID NO: 202 (DYGSWGYVSDIDY);
VLCDR1 comprises the sequence set forth in SEQ ID NO: 292 (SGSYITGSSVG);
VLCDR2 comprises the sequence set forth in SEQ ID NO: 284 (DNNDRPS); and
VLCDR3 comprises the sequence set forth in SEQ ID NO: 285 (ASYDTSNIGL);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 145 to 157 of SEQ ID NO: 1. The specific binding molecule comprising CDRs having 100% identity to those given above is referred to as “CC7” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 198 (SNAVI);
VHCDR2 comprises the sequence set forth in SEQ ID NO: 200 (LIDVDGDAAYDPALKS);
VHCDR3 comprises the sequence set forth in SEQ ID NO: 202 (DYGSWGYVSDIDY);
VLCDR1 comprises the sequence set forth in SEQ ID NO: 292 (SGSYITGSSVG);
VLCDR2 comprises the sequence set forth in SEQ ID NO: 284 (DNNDRPS); and
VLCDR3 comprises the sequence set forth in SEQ ID NO: 285 (ASYDTSNIGL).

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 519 (QVQLQESGPSLVKPSQTLSLTCTVSGFSLT);
VHFR2 comprises the sequence set forth in SEQ ID NO: 520 (WVRQAPGKAPEWVA);
VHFR3 comprises the sequence set forth in SEQ ID NO: 521 (RLSITRDTSKSQVSLSLRSVTTEDTAVYYCAR);
VHFR4 comprises the sequence set forth in SEQ ID NO: 522 (WGPGLLVTVSS);
VLFR1 comprises the sequence set forth in SEQ ID NO: 523 (RVVRTQPSSVSGSLGQRVSITC);
VLFR2 comprises the sequence set forth in SEQ ID NO: 524 (WFQQVPGSGLKTVIY);
VLFR3 comprises the sequence set forth in SEQ NO: 525 (GVPDRFSGSKSGDTATLTISSLQAEDEADYYC);
VLFR4 comprises the sequence set forth in SEQ ID NO: 526 (FGSGTRLTVLG);
or for each FR sequence, an amino acid sequence with
   (i) at least 50% identity thereto, and/or
   (ii) one, two, three, four or five amino acid substitutions relative thereto.

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 519 (QVQLQESGPSLVKPSQTLSLTCTVSGFSLT);
VHFR2 comprises the sequence set forth in SEQ ID NO: 520 (WVRQAPGKAPEWVA);
VHFR3 comprises the sequence set forth in SEQ ID NO: 521 (RLSITRDTSKSQVSLSLRSVTTEDTAVYYCAR);
VHFR4 comprises the sequence set forth in SEQ ID NO: 522 (WGPGLLVTVSS);
VLFR1 comprises the sequence set forth in SEQ ID NO: 523 (RVVRTQPSSVSGSLGQRVSITC);
VLFR2 comprises the sequence set forth in SEQ ID NO: 524 (WFQQVPGSGLKTVIY);
VLFR3 comprises the sequence set forth in SEQ ID NO: 525 (GVPDRFSGSKSGDTATLTISSLQAEDEADYYC);
VLFR4 comprises the sequence set forth in SEQ ID NO: 526 (FGSGTRLTVLG);
or for each FR sequence, an amino acid sequence with
   (i) at least 50% identity thereto, and/or
   (ii) one, two, three, four or five amino acid substitutions relative thereto,
wherein the specific binding molecule binds to a polypeptide or protein molecule comprising an amino acid sequence comprising residues 145 to 157 of SEQ ID NO: 1. A specific binding molecule comprising FRs having 100% identity to those given above is referred to as “CC7” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 519 (QVQLQESGPSLVKPSQTLSLTCTVSGFSLT);
   VHFR2 comprises the sequence set forth in SEQ ID NO: 520 (WVRQAPGKAPEWVA);
   VHFR3 comprises the sequence set forth in SEQ ID NO: 521 (RLSITRDTSKSQVSLSLRSVTTEDTAVYYCAR);
   VHFR4 comprises the sequence set forth in SEQ ID NO: 522 (WGPGLLVTVSS);
   VLFR1 comprises the sequence set forth in SEQ ID NO: 523 (RVVRTQPSSVSGSLGQRVSITC);
   VLFR2 comprises the sequence set forth in SEQ ID NO: 524 (WFQQVPGSGLKTVIY);
   VLFR3 comprises the sequence set forth in SEQ ID NO: 525 (GVPDRFSGSKSGDTATLTISSLQAEDEADYYC);
   VLFR4 comprises the sequence set forth in SEQ ID NO: 526 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 198 (SNAVI);
   VHCDR2 comprises the sequence set forth in SEQ ID NO: 200 (LIDVDGDAAYDPALKS);
   VHCDR3 comprises the sequence set forth in SEQ ID NO: 202 (DYGSWGYVSDIDY);
   VLCDR1 comprises the sequence set forth in SEQ ID NO: 292 (SGSYITGSSVG);
   VLCDR2 comprises the sequence set forth in SEQ ID NO: 284 (DNNDRPS); and
   VLCDR3 comprises the sequence set forth in SEQ ID NO: 285 (ASYDTSNIGL);
   or for each CDR sequence, an amino acid sequence with
      (i) at least 85% identity thereto, and/or
      (ii) one, two, or three amino acid substitutions relative thereto
   wherein the specific binding molecule binds to a polypeptide or protein molecule comprising an amino acid sequence comprising residues 145 to 157 of SEQ ID NO: 1. The specific binding molecule comprising FRs and CDRs having 100% identity to those given above is referred to as “CC7” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 519 (QVQLQESGPSLVKPSQTLSLTCTVSGFSLT);
   VHFR2 comprises the sequence set forth in SEQ ID NO: 520 (WVRQAPGKAPEWVA);
   VHFR3 comprises the sequence set forth in SEQ ID NO: 521 (RLSITRDTSKSQVSLSLRSVTTEDTAVYYCAR);
   VHFR4 comprises the sequence set forth in SEQ ID NO: 522 (WGPGLLVTVSS);
   VLFR1 comprises the sequence set forth in SEQ ID NO: 523 (RVVRTQPSSVSGSLGQRVSITC);
   VLFR2 comprises the sequence set forth in SEQ ID NO: 524 (WFQQVPGSGLKTVIY);
   VLFR3 comprises the sequence set forth in SEQ NO: 525 (GVPDRFSGSKSGDTATLTISSLQAEDEADYYC);
   VLFR4 comprises the sequence set forth in SEQ ID NO: 526 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 198 (SNAVI);
   VHCDR2 comprises the sequence set forth in SEQ ID NO: 200 (LIDVDGDAAYDPALKS);
   VHCDR3 comprises the sequence set forth in SEQ ID NO: 202 (DYGSWGYVSDIDY);
   VLCDR1 comprises the sequence set forth in SEQ ID NO: 292 (SGSYITGSSVG);
   VLCDR2 comprises the sequence set forth in SEQ ID NO: 284 (DNNDRPS); and
   VLCDR3 comprises the sequence set forth in SEQ ID NO: 285 (ASYDTSNIGL);
   wherein the specific binding molecule binds to a polypeptide or protein molecule comprising an amino acid sequence comprising residues 145 to 157 of SEQ ID NO: 1. The specific binding molecule comprising FRs and CDRs having 100% identity to those given above is referred to as “CC7” herein.

The specific binding molecule may comprise:
(a) A VH domain comprising the sequence set forth in SEQ ID NO: 527 (QVQLQESGPSLVKPSQTLSLTCTVSGFSLTSNAVIWVRQAPGKAPEWVALIDVDGDAAY DPALKSRLSITRDTSKSQVSLSLRSVTTEDTAVYYCARDYGSWGYVSDIDYWGPGLLVTV SS); and/or
(b) a VL domain comprising the sequence set forth in SEQ ID NO: 528 (RVVRTQPSSVSGSLGQRVSITCSGSYITGSSVGWFQQVPGSGLKTVIYDNNDRPSGVPD RFSGSKSGDTATLTISSLQAEDEADYYCASYDTSNIGLFGSGTRLTVLG);
or a humanized variant thereof.

The specific binding molecule may comprise:
(a) A heavy chain comprising the sequence set forth in SEQ ID NO: 529 (QVQLQESGPSLVKPSQTLSLTCTVSGFSLTSNAVIWVRQAPGKAPEWVALIDVDGDAAY DPALKSRLSITRDTSKSQVSLSLRSVTTEDTAVYYCARDYGSWGYVSDIDYWGPGLLVTV SSAKTTAPSVYPLAPVCGDTTGSSVTLGCLVKGYFPEPVTLTWNSGSLSSGVHTFPAVLQ SDLYTLSSSVTVTSSTWPSQSITCNVAHPASSTKVDKKIEPRGPTIKPCPPCKCPAPNLLG GPSVFIFPPKIKDVLMISLSPIVTCVVVDVSEDDPDVQISWFVNNVEVHTAQTQTHREDYN STLRVVSALPIQHQDWMSGKEFKCKVNNKDLPAPIERTISKPKGSVRAPQVYVLPPPEEE MTKKQVTLTCMVTDFMPEDIYVEWTNNGKTELNYKNTEPVLDSDGSYFMYSKLRVEKKN WVERNSYSCSVVHEGLHNHHTTKSFSRTPGK); and/or
(b) a light chain comprising the sequence set forth in SEQ ID NO: 530 (RVVRTQPSSVSGSLGQRVSITCSGSYITGSSVGWFQQVPGSGLKTVIYDNNDRPSGVPD RFSGSKSGDTATLTISSLQAEDEADYYCASYDTSNIGLFGSGTRLTVLGGQPKSSPSVTLF PPSSEELETNKATLVCTITDFYPGVVTVDWKVDGTPVTQGMETTQPSKQSNNKYMASSY LTLTARAWERHSSYSCQVTHEGHTVEKSLSRADCS);
or a humanized variant thereof.

The epitope of the specific binding molecule may be within an amino acid sequence comprising residues 155 to 227 of SEQ ID NO: 1. Accordingly, the epitope may be within the amino acid 294 sequence of SEQ ID NO: (RGAAPPGQKGQANATRIPAKTPPAPKTPPSSGEPPKSGDRSGYSSPGSPGTPGSRSRTPSLPTP PTREPKKVA).

The epitope may be within an amino acid sequence comprising residues 155 to 227 of SEQ ID NO: 1. This epitope may be bound by the CDRs of the specific binding molecules referred to as “CB12” and “CC3” herein.

The epitope may comprise the amino acid sequence of SEQ ID NO: 294 (RGAAPPGQKGQANATRIPAKTPPAPKTPPSSGEPPKSGDRSGYSSPGSPGTPGSRSRTPSLPTP PTREPKKVA). The epitope may comprise an amino acid sequence having at least 70%, at least 75%, at least 80%, at least 90%, at least 95% or at least 99% identity to SEQ ID NO: 294 (RGAAPPGQKGQANATRIPAKTPPAPKTPPSSGEPPKSGDRSGYSSPGSPGTPGSRSRTPSLPTP PTREPKKVA).

The epitope may consist of the amino acid sequence of SEQ ID NO: 294 (RGAAPPGQKGQANATRIPAKTPPAPKTPPSSGEPPKSGDRSGYSSPGSPGTPGSRSRTPSLPTP PTREPKKVA). The epitope may consist of an amino acid sequence having at least 70%, at least 75%, at least 80%, at least 90%, at least 95% or at least 99% identity to SEQ ID NO: 294 (RGAAPPGQKGQANATRIPAKTPPAPKTPPSSGEPPKSGDRSGYSSPGSPGTPGSRSRTPSLPTP PTREPKKVA).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 259 (SNGVG);
VHCDR2 comprises the sequence set forth in SEQ ID NO: 182 (DKSSAGKTYGNPALKS);
VHCDR3 comprises the sequence set forth in SEQ ID NO: 286 (CRDGGVSYGYDVDY);
VLCDR1 comprises the sequence set forth in SEQ ID NO: 287 (SGSSSNVGGDYVG);
VLCDR2 comprises the sequence set forth in SEQ ID NO: 288 (DTTSRPS); and
VLCDR3 comprises the sequence set forth in SEQ ID NO: 289 (ASVDKTTGGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 155 to 227 of SEQ ID NO: 1. The specific binding molecule comprising CDRs having 100% identity to those given above is referred to as “CB12” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 259 (SNGVG);
VHCDR2 comprises the sequence set forth in SEQ ID NO: 182 (DKSSAGKTYGNPALKS);
VHCDR3 comprises the sequence set forth in SEQ ID NO: 286 (CRDGGVSYGYDVDY);
VLCDR1 comprises the sequence set forth in SEQ ID NO: 287 (SGSSSNVGGDYVG);
VLCDR2 comprises the sequence set forth in SEQ ID NO: 288 (DTTSRPS); and
VLCDR3 comprises the sequence set forth in SEQ ID NO: 289 (ASVDKTTGGV).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 259 (SNGVG);
VHCDR2 comprises the sequence set forth in SEQ ID NO: 182 (DKSSAGKTYGNPALKS);
VHCDR3 comprises the sequence set forth in SEQ ID NO: 286 (CRDGGVSYGYDVDY);
VLCDR1 comprises the sequence set forth in SEQ ID NO: 290 (SGSSSNVGYGTYVS);
VLCDR2 comprises the sequence set forth in SEQ ID NO: 188 (GTTTRAS); and
VLCDR3 comprises the sequence set forth in SEQ ID NO: 291 (ASYDTGSGGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 155 to 227 of SEQ ID NO: 1. The specific binding molecule comprising CDRs having 100% identity to those given above is referred to as “CC3” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 259 (SNGVG);
VHCDR2 comprises the sequence set forth in SEQ ID NO: 182 (DKSSAGKTYGNPALKS);
VHCDR3 comprises the sequence set forth in SEQ ID NO: 286 (CRDGGVSYGYDVDY);
VLCDR1 comprises the sequence set forth in SEQ ID NO: 290 (SGSSSNVGYGTYVS);
VLCDR2 comprises the sequence set forth in SEQ ID NO: 188 (GTTTRAS); and
VLCDR3 comprises the sequence set forth in SEQ ID NO: 291 (ASYDTGSGGV).

The epitope of the specific binding molecule may be within an amino acid sequence comprising residues 155 to 238 of SEQ ID NO: 1. Accordingly, the epitope may be within the amino acid sequence of SEQ ID NO: 295 (RGAAPPGQKGQANATRIPAKTPPAPKTPPSSGEPPKSGDRSGYSSPGSPGTPGSRSRTPSLPTP PTREPKKVAVVRTPPKSPSS).

The epitope may be within an amino acid sequence comprising residues 155 to 238 of SEQ ID NO: 1. This epitope may be bound by the CDRs of the specific binding molecule referred to as “CA1” herein.

The epitope may comprise the amino acid sequence of SEQ ID NO: 295 (RGAAPPGQKGQANATRIPAKTPPAPKTPPSSGEPPKSGDRSGYSSPGSPGTPGSRSRTPSLPTP PTREPKKVAVVRTPPKSPSS). The epitope may comprise an amino acid sequence having at least 70%, at least 75%, at least 80%, at least 90%, at least 95% or at least 99% identity to SEQ ID NO: 295 (RGAAPPGQKGQANATRIPAKTPPAPKTPPSSGEPPKSGDRSGYSSPGSPGTPGSRSRTPSLPTP PTREPKKVAVVRTPPKSPSS).

The epitope may consist of the amino acid sequence of SEQ ID NO: 295 (RGAAPPGQKGQANATRIPAKTPPAPKTPPSSGEPPKSGDRSGYSSPGSPGTPGSRSRTPSLPTP PTREPKKVAVVRTPPKSPSS). The epitope may consist of an amino acid sequence having at least 70%, at least 75%, at least 80%, at least 90%, at least 95% or at least 99% identity to SEQ ID NO: 295 (RGAAPPGQKGQANATRIPAKTPPAPKTPPSSGEPPKSGDRSGYSSPGSPGTPGSRSRTPSLPTP PTREPKKVAVVRTPPKSPSS).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 198 (SNAVI);
VHCDR2 comprises the sequence set forth in SEQ ID NO: 296 (DIRADGATNYNAALKS);
VHCDR3 comprises the sequence set forth in SEQ ID NO: 297 (PGNYYYGAGRDVARLAD);
VLCDR1 comprises the sequence set forth in SEQ ID NO: 298 (SGSSSNIGGGNAVG);
VLCDR2 comprises the sequence set forth in SEQ ID NO: 288 (DTTSRPS); and
VLCDR3 comprises the sequence set forth in SEQ ID NO: 299 (AAMDSSSLIGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 155 to 238 of SEQ ID NO: 1. The specific binding molecule comprising CDRs having 100% identity to those given above is referred to as “CA1” herein.

The epitope of the specific binding molecule may be within an amino acid sequence comprising residues 186 to 263 of SEQ ID NO: 1. Accordingly, the epitope may be within the amino acid sequence of SEQ ID NO: 300 (GEPPKSGDRSGYSSPGSPGTPGSRSRTPSLPTPPTREPKKVAVVRTPPKSPSSAKSRLQTAPVP MPDLKNVKSKIGST).

The epitope may be within an amino acid sequence comprising residues 186 to 263 of SEQ ID NO: 1. This epitope may be bound by the CDRs of the specific binding molecule referred to as “CA3” herein.

The epitope may comprise the amino acid sequence of SEQ ID NO: 300 (GEPPKSGDRSGYSSPGSPGTPGSRSRTPSLPTPPTREPKKVAVVRTPPKSPSSAKSRLQT APVPMPDLKNVKSKIGST). The epitope may comprise an amino acid sequence having at least 70%, at least 75%, at least 80%, at least 90%, at least 95% or at least 99% identity to SEQ ID NO: 300 (GEPPKSGDRSGYSSPGSPGTPGSRSRTPSLPTPPTREPKKVAVVRTPPKSPSSAKSRLQT APVPMPDLKNVKSKIGST).

The epitope may consist of the amino acid sequence of SEQ ID NO: 300 (GEPPKSGDRSGYSSPGSPGTPGSRSRTPSLPTPPTREPKKVAVVRTPPKSPSSAKSRLQT APVPMPDLKNVKSKIGST). The epitope may consist of an amino acid sequence having at least 70%, at least 75%, at least 80%, at least 90%, at least 95% or at least 99% identity to SEQ ID NO: 300 (GEPPKSGDRSGYSSPGSPGTPGSRSRTPSLPTPPTREPKKVAVVRTPPKSPSSAKSRLQT APVPMPDLKNVKSKIGST).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 259 (SNGVG);
VHCDR2 comprises the sequence set forth in SEQ ID NO: 182 (DKSSAGKTYGNPALKS);
VHCDR3 comprises the sequence set forth in SEQ ID NO: 184 (CRDGGVTYGYDVDY);
VLCDR1 comprises the sequence set forth in SEQ ID NO: 301 (SGSSGNIGYDDYVS);
VLCDR2 comprises the sequence set forth in SEQ ID NO: 302 (GATRRSS); and
VLCDR3 comprises the sequence set forth in SEQ ID NO: 303 (ASYDSSGGGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 186 to 263 of SEQ ID NO: 1. The specific binding molecule comprising CDRs having 100% identity to those given above is referred to as “CA3” herein.

The epitope of the specific binding molecule may be within an amino acid sequence comprising residues 186 to 350 of SEQ ID NO: 1. Accordingly, the epitope may be within the amino acid sequence of SEQ ID NO: 304 (GEPPKSGDRSGYSSPGSPGTPGSRSRTPSLPTPPTREPKKVAVVRTPPKSPSSAKSRLQTAPVP MPDLKNVKSKIGSTENLKHQPGGGKVQIINKKLDLSNVQSKCGSKDNIKHVPGGGSVQIVYKPVDL SKVTSKCGSLGNIHHKPGGGQVEVKSEKLDFKDRV).

The epitope may be within an amino acid sequence comprising residues 186 to 350 of SEQ ID NO: 1. This epitope may be bound by the CDRs of the specific binding molecule referred to as “CD2” herein.

The epitope may comprise the amino acid sequence of SEQ ID NO: 304 (GEPPKSGDRSGYSSPGSPGTPGSRSRTPSLPTPPTREPKKVAVVRTPPKSPSSAKSRLQTAPVP MPDLKNVKSKIGSTENLKHQPGGGKVQIINKKLDLSNVQSKCGSKDNIKHVPGGGSVQIVYKPVDL SKVTSKCGSLGNIHHKPGGGQVEVKSEKLDFKDRV). The epitope may comprise an amino acid sequence having at least 70%, at least 75%, at least 80%, at least 90%, at least 95% or at least 99% identity to SEQ ID NO: 304 (GEPPKSGDRSGYSSPGSPGTPGSRSRTPSLPTPPTREPKKVAVVRTPPKSPSSAKSRLQTAPVP MPDLKNVKSKIGSTENLKHQPGGGKVQIINKKLDLSNVQSKCGSKDNIKHVPGGGSVQIVYKPVDL SKVTSKCGSLGNIHHKPGGGQVEVKSEKLDFKDRV).

The epitope may consist of the amino acid sequence of SEQ ID NO: 304 (GEPPKSGDRSGYSSPGSPGTPGSRSRTPSLPTPPTREPKKVAVVRTPPKSPSSAKSRLQTAPVP MPDLKNVKSKIGSTENLKHQPGGGKVQIINKKLDLSNVQSKCGSKDNIKHVPGGGSVQIVYKPVDL SKVTSKCGSLGNIHHKPGGGQVEVKSEKLDFKDRV). The epitope may consist of an amino acid sequence having at least 70%, at least 75%, at least 80%, at least 90%, at least 95% or at least 99% identity to SEQ ID NO: 304 (GEPPKSGDRSGYSSPGSPGTPGSRSRTPSLPTPPTREPKKVAVVRTPPKSPSSAKSRLQTAPVP MPDLKNVKSKIGSTENLKHQPGGGKVQIINKKLDLSNVQSKCGSKDNIKHVPGGGSVQIVYKPVDL SKVTSKCGSLGNIHHKPGGGQVEVKSEKLDFKDRV).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 198 (SNAVI);
VHCDR2 comprises the sequence set forth in SEQ ID NO: 200 (LIDVDGDAAYDPALKS);
VHCDR3 comprises the sequence set forth in SEQ ID NO: 202 (DYGSWGYVSDIDY);
VLCDR1 comprises the sequence set forth in SEQ ID NO: 305 (SGSNIGDADVG);
VLCDR2 comprises the sequence set forth in SEQ ID NO: 306 (YNENRPS); and
VLCDR3 comprises the sequence set forth in SEQ ID NO: 307 (GSYAGDTYNHGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 186 to 350 of SEQ ID NO: 1. The specific binding molecule comprising CDRs having 100% identity to those given above is referred to as “CD2” herein.

The epitope of the specific binding molecule may be within an amino acid sequence comprising residues 239 to 348 of SEQ ID NO: 1. Accordingly, the epitope may be within the amino acid sequence of SEQ ID NO: 308 (AKSRLQTAPVPMPDLKNVKSKIGSTENLKHQPGGGKVQIINKKLDLSNVQSKCGSKDNIKHVPGG GSVQIVYKPVDLSKVTSKCGSLGNIHHKPGGGQVEVKSEKLDFKD).

The epitope may be within an amino acid sequence comprising residues 239 to 348 of SEQ ID NO: 1. This epitope may be bound by the CDRs of the specific binding molecule referred to as “CB9” herein.

The epitope may comprise the amino acid sequence of 308 (AKSRLQTAPVPMPDLKNVKSKIGSTENLKHQPGGGKVQIINKKLDLSNVQSKCGSKDNIKHVPGG GSVQIVYKPVDLSKVTSKCGSLGNIHHKPGGGQVEVKSEKLDFKD). The epitope may comprise an amino acid sequence having at least 70%, at least 75%, at least 80%, at least 90%, at least 95% or at least 99% identity to 308 (AKSRLQTAPVPMPDLKNVKSKIGSTENLKHQPGGGKVQIINKKLDLSNVQSKCGSKDNIKHVPGG GSVQIVYKPVDLSKVTSKCGSLGNIHHKPGGGQVEVKSEKLDFKD).

The epitope may consist of the amino acid sequence of 308 (AKSRLQTAPVPMPDLKNVKSKIGSTENLKHQPGGGKVQIINKKLDLSNVQSKCGSKDNIK HVPGGGSVQIVYKPVDLSKVTSKCGSLGNIHHKPGGGQVEVKSEKLDFKD). The epitope may consist of an amino acid sequence having at least 70%, at least 75%, at least 80%, at least 90%, at 95% at least 99% least or identity to 308 (AKSRLQTAPVPMPDLKNVKSKIGSTENLKHQPGGGKVQIINKKLDLSNVQSKCGSKDNIKHVPGG GSVQIVYKPVDLSKVTSKCGSLGNIHHKPGGGQVEVKSEKLDFKD).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 48 (GIDSDGEEGYNPALNS);
VHCDR3 comprises the sequence set forth in SEQ ID NO: 54 (SYRADGLAYGYVQAIDY);
VLCDR1 comprises the sequence set forth in SEQ ID NO: 67 (SGRFIGISSVG);
VLCDR2 comprises the sequence set forth in SEQ ID NO: 70 (ASDGRPS); and
VLCDR3 comprises the sequence set forth in SEQ ID NO: 74 (GSSDRTQYTGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 239 to 348 of SEQ ID NO: 1. The specific binding molecule comprising CDRs having 100% identity to those given above is referred to as “CB9” herein.

The epitope of the specific binding molecule may be within an amino acid sequence comprising residues 266 to 359 of SEQ ID NO: 1. Accordingly, the epitope may be within the amino acid sequence of SEQ ID NO: 309 (LKHQPGGGKVQIINKKLDLSNVQSKCGSKDNIKHVPGGGSVQIVYKPVDLSKVTSKCGSLGNIHH KPGGGQVEVKSEKLDFKDRVQSKIGSLDN).

The epitope may be within an amino acid sequence comprising residues 266 to 359 of SEQ ID NO: 1. This epitope may be bound by the CDRs of the specific binding molecule referred to as “CG11” herein.

The epitope may comprise the amino acid sequence of SEQ ID NO: 309 (LKHQPGGGKVQIINKKLDLSNVQSKCGSKDNIKHVPGGGSVQIVYKPVDLSKVTSKCGSLGNIHH KPGGGQVEVKSEKLDFKDRVQSKIGSLDN). The epitope may comprise an amino acid sequence having at least 70%, at least 75%, at least 80%, at least 90%, at least 95% or at least 99% identity to SEQ ID NO: 309 (LKHQPGGGKVQIINKKLDLSNVQSKCGSKDNIKHVPGGGSVQIVYKPVDLSKVTSKCGSLGNIHH KPGGGQVEVKSEKLDFKDRVQSKIGSLDN).

The epitope may consist of the amino acid sequence of SEQ ID NO: 309 (LKHQPGGGKVQIINKKLDLSNVQSKCGSKDNIKHVPGGGSVQIVYKPVDLSKVTSKCGSLGNIHH KPGGGQVEVKSEKLDFKDRVQSKIGSLDN). The epitope may consist of an amino acid sequence having at least 70%, at least 75%, at least 80%, at least 90%, at least 95% or at least 99% identity to SEQ ID NO: 309 (LKHQPGGGKVQIINKKLDLSNVQSKCGSKDNIKHVPGGGSVQIVYKPVDLSKVTSKCGSLGNIHH KPGGGQVEVKSEKLDFKDRVQSKIGSLDN).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 310 (NYPVG);
VHCDR2 comprises the sequence set forth in SEQ ID NO: 311 (NIENDGSANYASALKS);
VHCDR3 comprises the sequence set forth in SEQ ID NO: 312 (EFGGSDGYTYFVDIDY);
VLCDR1 comprises the sequence set forth in SEQ ID NO: 313 (SGSSSNVGYGNYVS);
VLCDR2 comprises the sequence set forth in SEQ ID NO: 141 (GATSRAS); and
VLCDR3 comprises the sequence set forth in SEQ ID NO: 314 (ASYDGSSSGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 266 to 359 of SEQ ID NO: 1. The specific binding molecule comprising CDRs having 100% identity to those given above is referred to as “CG11” herein.

The epitope of the specific binding molecule may be within an amino acid sequence comprising residues 277 to 319 of SEQ ID NO: 1. Accordingly, the epitope may be within the amino acid sequence of SEQ ID NO: 315 (IINKKLDLSNVQSKCGSKDNIKHVPGGGSVQIVYKPVDLSKVT).

The epitope may be within an amino acid sequence comprising residues 277 to 319 of SEQ ID NO: 1. This epitope may be bound by the CDRs of the specific binding molecule referred to as “CA10” herein.

The epitope may comprise the amino acid sequence of SEQ ID NO: 315 (IINKKLDLSNVQSKCGSKDNIKHVPGGGSVQIVYKPVDLSKVT). The epitope may comprise an amino acid sequence having at least 70%, at least 75%, at least 80%, at least 90%, at least 95% or at least 99% identity to SEQ ID NO: 315 (IINKKLDLSNVQSKCGSKDNIKHVPGGGSVQIVYKPVDLSKVT).

The epitope may consist of the amino acid sequence of SEQ ID NO: 315 (IINKKLDLSNVQSKCGSKDNIKHVPGGGSVQIVYKPVDLSKVT). The epitope may consist of an amino acid sequence having at least 70%, at least 75%, at least 80%, at least 90%, at least 95% or at least 99% identity to SEQ ID NO: 315 (IINKKLDLSNVQSKCGSKDNIKHVPGGGSVQIVYKPVDLSKVT).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 259 (SNGVG);
VHCDR2 comprises the sequence set forth in SEQ ID NO: 157 (DISSVGKKYANPALKS);
VHCDR3 comprises the sequence set forth in SEQ ID NO: 161 (CRDGGVTYGYDIDY);
VLCDR1 comprises the sequence set forth in SEQ ID NO: 316 (SGSSSNVGYGNYVT);
VLCDR2 comprises the sequence set forth in SEQ ID NO: 317 (DATTRVS); and
VLCDR3 comprises the sequence set forth in SEQ ID NO: 318 (AAHDSSSGGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 277 to 319 of SEQ ID NO: 1. The specific binding molecule comprising CDRs having 100% identity to those given above is referred to as “CA10” herein.

The epitope of the specific binding molecule may be within an amino acid sequence comprising residues 297 to 390 of SEQ ID NO: 1. Accordingly, the epitope may be within the amino acid sequence of SEQ ID NO: 5 (IKHVPGGGSVQIVYKPVDLSKVTSKCGSLGNIHHKPGGGQVEVKSEKLDFKDRVQSKIGSLDNITH VPGGGNKKIETHKLTFRENAKAKTDHGA).

The epitope may be within an amino acid sequence comprising residues 297 to 390 of SEQ ID NO: 1. This epitope may be bound by the CDRs of the specific binding molecule referred to as “CC12” herein.

The epitope may comprise the amino acid sequence of SEQ ID NO: 5 (IKHVPGGGSVQIVYKPVDLSKVTSKCGSLGNIHHKPGGGQVEVKSEKLDFKDRVQSKIGSLDNITH VPGGGNKKIETHKLTFRENAKAKTDHGA). The epitope may comprise an amino acid sequence having at least 70%, at least 75%, at least 80%, at least 90%, at least 95% or at least 99% identity to SEQ ID NO: 5 (IKHVPGGGSVQIVYKPVDLSKVTSKCGSLGNIHHKPGGGQVEVKSEKLDFKDRVQSKIGSLDNITH VPGGGNKKIETHKLTFRENAKAKTDHGA).

The epitope may consist of the amino acid sequence of SEQ ID NO: 5 (IKHVPGGGSVQIVYKPVDLSKVTSKCGSLGNIHHKPGGGQVEVKSEKLDFKDRVQSKIGSLDNITH VPGGGNKKIETHKLTFRENAKAKTDHGA). The epitope may consist of an amino acid sequence having at least 70%, at least 75%, at least 80%, at least 90%, at least 95% or at least 99% identity to SEQ ID NO: 5 (IKHVPGGGSVQIVYKPVDLSKVTSKCGSLGNIHHKPGGGQVEVKSEKLDFKDRVQSKIGSLDNITH VPGGGNKKIETHKLTFRENAKAKTDHGA).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 48 (GIDSDGEEGYNPALNS);
VHCDR3 comprises the sequence set forth in SEQ ID NO: 54 (SYRADGLAYGYVQAIDY);
VLCDR1 comprises the sequence set forth in SEQ ID NO: 67 (SGRFIGISSVG);
VLCDR2 comprises the sequence set forth in SEQ ID NO: 70 (ASDGRPS); and
VLCDR3 comprises the sequence set forth in SEQ ID NO: 74 (GSSDRTQYTGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 297 to 390 of SEQ ID NO: 1. The specific binding molecule comprising CDRs having 100% identity to those given above is referred to as “CC12” herein.

The epitope of the specific binding molecule may be within an amino acid sequence comprising residues 319 to 331 of SEQ ID NO: 1. Accordingly, the epitope may be within the amino acid sequence of SEQ ID NO: 319 (TSKCGSLGNIHHK).

The epitope may be within an amino acid sequence comprising residues 319 to 331 of SEQ ID NO: 1. This epitope may be bound by the CDRs of the specific binding molecule referred to as “CE2” or “E1B8” herein. Critical residues of the epitope may be residues 323 (G), 324 (S), 325 (L), 326 (G), 327 (N) and/or 328 (I) (numbering according to SEQ ID NO: 1). The epitope may comprise the amino acid sequence of SEQ ID NO: 320 (XXXXGSLGNIXXX, wherein X is any amino acid). The epitope may comprise the amino acid sequence of SEQ ID NO: 319, wherein any one or more residue other than residue number 323 (G), 324 (S), 325 (L), 326 (G), 327 (N) and/or 328 (I) is replaced by a non-conservative amino acid substitution (numbering according to SEQ ID NO: 1). The epitope may comprise the amino acid sequence of SEQ ID NO: 319, wherein any one or more residue other than residue number 323 (G), 324 (S), 325 (L), 326 (G), 327 (N) and/or 328 (I) is replaced by a conservative amino acid substitution (numbering according to SEQ ID NO: 1). The epitope may comprise the amino acid sequence of SEQ ID NO: 319, wherein any one or more residue other than residue number 323 (G), 324 (S), 325 (L), 326 (G), 327 (N) and/or 328 (I) is replaced by a conservative amino acid substitution (numbering according to SEQ ID NO: 1) and any one or more residue other than residue number 323 (G), 324 (S), 325 (L), 326 (G), 327 (N) and/or 328 (I) is replaced by a non-conservative amino acid substitution (numbering according to SEQ ID NO: 1).

The epitope may comprise the amino acid sequence of SEQ ID NO: 319 (TSKCGSLGNIHHK). The epitope may comprise an amino acid sequence having at least 70%, at least 75%, at least 80%, at least 90%, at least 95% or at least 99% identity to SEQ ID NO: 319 (TSKCGSLGNIHHK).

The epitope may consist of the amino acid sequence of SEQ ID NO: 319 (TSKCGSLGNIHHK). The epitope may consist of an amino acid sequence having at least 70%, at least 75%, at least 80%, at least 90%, at least 95% or at least 99% identity to SEQ ID NO: 319 (TSKCGSLGNIHHK).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 310 (NYPVG);
VHCDR2 comprises the sequence set forth in SEQ ID NO: 311 (NIENDGSANYASALKS);
VHCDR3 comprises the sequence set forth in SEQ ID NO: 312 (EFGGSDGYTYFVDIDY);
VLCDR1 comprises the sequence set forth in SEQ ID NO: 313 (SGSSSNVGYGNYVS);
VLCDR2 comprises the sequence set forth in SEQ ID NO: 141 (GATSRAS); and
VLCDR3 comprises the sequence set forth in SEQ ID NO: 314 (ASYDGSSSGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 319 to 331 of SEQ ID NO: 1. The specific binding molecule comprising CDRs having 100% identity to those given above is referred to as “CE2” or “E1B8” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 310 (NYPVG);
VHCDR2 comprises the sequence set forth in SEQ ID NO: 311 (NIENDGSANYASALKS);
VHCDR3 comprises the sequence set forth in SEQ ID NO: 312 (EFGGSDGYTYFVDIDY);
VLCDR1 comprises the sequence set forth in SEQ ID NO: 313 (SGSSSNVGYGNYVS);
VLCDR2 comprises the sequence set forth in SEQ ID NO: 141 (GATSRAS); and
VLCDR3 comprises the sequence set forth in SEQ ID NO: 314 (ASYDGSSSGV).

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 531 (QVQLQESGPSLVKPSQTLSLTCTVSGFSLT);
VHFR2 comprises the sequence set forth in SEQ ID NO: 532 (WVRQAPGKALEWIG);
VHFR3 comprises the sequence set forth in SEQ ID NO: 533 (RLSITRDTSKNQVSLSLSSATTEDTAVYYCGR);
VHFR4 comprises the sequence set forth in SEQ ID NO: 534 (WGPGLLVTVSS);
VLFR1 comprises the sequence set forth in SEQ ID NO: 535 (QAVLTQPSSVSKSLGQSVSITC);
VLFR2 comprises the sequence set forth in SEQ ID NO: 536 (WFQQVPGSAPKILIY);
VLFR3 comprises the sequence set forth in SEQ ID NO: 537 (GVPDRFSGSRSGNTATLTITSLQAEDEADYYC);
VLFR4 comprises the sequence set forth in SEQ ID NO: 538 (FGSGTRLTVLG);
or for each FR sequence, an amino acid sequence with
   (i) at least 50% identity thereto, and/or
   (ii) one, two, three, four or five amino acid substitutions relative thereto.

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 531 (QVQLQESGPSLVKPSQTLSLTCTVSGFSLT);
VHFR2 comprises the sequence set forth in SEQ ID NO: 532 (WVRQAPGKALEWIG);
VHFR3 comprises the sequence set forth in SEQ ID NO: 533 (RLSITRDTSKNQVSLSLSSATTEDTAVYYCGR);
VHFR4 comprises the sequence set forth in SEQ ID NO: 534 (WGPGLLVTVSS);
VLFR1 comprises the sequence set forth in SEQ ID NO: 535 (QAVLTQPSSVSKSLGQSVSITC);
VLFR2 comprises the sequence set forth in SEQ ID NO: 536 (WFQQVPGSAPKILIY);
VLFR3 comprises the sequence set forth in SEQ ID NO: n537 (GVPDRFSGSRSGNTATLTITSLQAEDEADYYC);
VLFR4 comprises the sequence set forth in SEQ ID NO: 538 (FGSGTRLTVLG);
or for each FR sequence, an amino acid sequence with
   (i) at least 50% identity thereto, and/or
   (ii) one, two, three, four or five amino acid substitutions relative thereto,
wherein the specific binding molecule binds to a polypeptide or protein molecule comprising an amino acid sequence comprising residues 319 to 331 of SEQ ID NO: 1. A specific binding molecule comprising FRs having 100% identity to those given above is referred to as “CE2” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 531 (QVQLQESGPSLVKPSQTLSLTCTVSGFSLT);
   VHFR2 comprises the sequence set forth in SEQ ID NO: 532 (WVRQAPGKALEWIG);
   VHFR3 comprises the sequence set forth in SEQ ID NO: 533 (RLSITRDTSKNQVSLSLSSATTEDTAVYYCGR);
   VHFR4 comprises the sequence set forth in SEQ ID NO: 534 (WGPGLLVTVSS);
   VLFR1 comprises the sequence set forth in SEQ ID NO: 535 (QAVLTQPSSVSKSLGQSVSITC);
   VLFR2 comprises the sequence set forth in SEQ ID NO: 536 (WFQQVPGSAPKILIY);
   VLFR3 comprises the sequence set forth in SEQ ID NO: 537 (GVPDRFSGSRSGNTATLTITSLQAEDEADYYC);
   VLFR4 comprises the sequence set forth in SEQ ID NO: 538 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 310 (NYPVG);
   VHCDR2 comprises the sequence set forth in SEQ ID NO: 311 (NIENDGSANYASALKS);
   VHCDR3 comprises the sequence set forth in SEQ ID NO: 312 (EFGGSDGYTYFVDIDY);
   VLCDR1 comprises the sequence set forth in SEQ ID NO: 313 (SGSSSNVGYGNYVS);
   VLCDR2 comprises the sequence set forth in SEQ ID NO: 141 (GATSRAS); and
   VLCDR3 comprises the sequence set forth in SEQ ID NO: 314 (ASYDGSSSGV);
   or for each CDR sequence, an amino acid sequence with
      (i) at least 85% identity thereto, and/or
      (ii) one, two, or three amino acid substitutions relative thereto
   wherein the specific binding molecule binds to a polypeptide or protein molecule comprising an amino acid sequence comprising residues 319 to 331 of SEQ ID NO: 1. The specific binding molecule comprising FRs and CDRs having 100% identity to those given above is referred to as “CE2” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 531 (QVQLQESGPSLVKPSQTLSLTCTVSGFSLT);
   VHFR2 comprises the sequence set forth in SEQ ID NO: 532 (WVRQAPGKALEWIG);
   VHFR3 comprises the sequence set forth in SEQ ID NO: 533 (RLSITRDTSKNQVSLSLSSATTEDTAVYYCGR);
   VHFR4 comprises the sequence set forth in SEQ ID NO: 534 (WGPGLLVTVSS);
   VLFR1 comprises the sequence set forth in SEQ ID NO: 535 (QAVLTQPSSVSKSLGQSVSITC);
   VLFR2 comprises the sequence set forth in SEQ ID NO: 536 (WFQQVPGSAPKILIY);
   VLFR3 comprises the sequence set forth in SEQ ID NO: 537 (GVPDRFSGSRSGNTATLTITSLQAEDEADYYC);
   VLFR4 comprises the sequence set forth in SEQ ID NO: 538 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 310 (NYPVG);
   VHCDR2 comprises the sequence set forth in SEQ ID NO: 311 (NIENDGSANYASALKS);
   VHCDR3 comprises the sequence set forth in SEQ ID NO: 312 (EFGGSDGYTYFVDIDY);
   VLCDR1 comprises the sequence set forth in SEQ ID NO: 313 (SGSSSNVGYGNYVS);
   VLCDR2 comprises the sequence set forth in SEQ ID NO: 141 (GATSRAS); and
   VLCDR3 comprises the sequence set forth in SEQ ID NO: 314 (ASYDGSSSGV);
   wherein the specific binding molecule binds to a polypeptide or protein molecule comprising an amino acid sequence comprising residues 319 to 331 of SEQ ID NO: 1. The specific binding molecule comprising FRs and CDRs having 100% identity to those given above is referred to as “CE2” herein.

The specific binding molecule may comprise:
(a) A VH domain comprising the sequence set forth in SEQ ID NO: 539 (QVQLQESGPSLVKPSQTLSLTCTVSGFSLTNYPVGWVRQAPGKALEWIGNIENDGSANY ASALKSRLSITRDTSKNQVSLSLSSATTEDTAVYYCGREFGGSDGYTYFVDIDYWGPGLL VTVSS); and/or
(b) a VL domain comprising the sequence set forth in SEQ ID NO: 540 (QAVLTQPSSVSKSLGQSVSITCSGSSSNVGYGNYVSWFQQVPGSAPKILIYGATSRAS GVPDRFSGSRSGNTATLTITSLQAEDEADYYCASYDGSSSGVFGSGTRLTVLG);
or a humanized variant thereof.

The specific binding molecule may comprise:
(a) A heavy chain comprising the sequence set forth in SEQ ID NO: 541 (QVQLQESGPSLVKPSQTLSLTCTVSGFSLTNYPVGWVRQAPGKALEWIGNIENDGSANY ASALKSRLSITRDTSKNQVSLSLSSATTEDTAVYYCGREFGGSDGYTYFVDIDYWGPGLL VTVSSAKTTAPSVYPLAPVCGDTTGSSVTLGCLVKGYFPEPVTLTWNSGSLSSGVHTFPA VLQSDLYTLSSSVTVTSSTWPSQSITCNVAHPASSTKVDKKIEPRGPTIKPCPPCKCPAPN LLGGPSVFIFPPKIKDVLMISLSPIVTCVVVDVSEDDPDVQISWFVNNVEVHTAQTQTHRED YNSTLRVVSALPIQHQDWMSGKEFKCKVNNKDLPAPIERTISKPKGSVRAPQVYVLPPPE EEMTKKQVTLTCMVTDFMPEDIYVEWTNNGKTELNYKNTEPVLDSDGSYFMYSKLRVEK KNWVERNSYSCSVVHEGLHNHHTTKSFSRTPGK); and/or
(b) a light chain comprising the sequence set forth in SEQ ID NO: 542 (QAVLTQPSSVSKSLGQSVSITCSGSSSNVGYGNYVSWFQQVPGSAPKILIYGATSRASG VPDRFSGSRSGNTATLTITSLQAEDEADYYCASYDGSSSGVFGSGTRLTVLGGQPKSSP SVTLFPPSSEELETNKATLVCTITDFYPGVVTVDWKVDGTPVTQGMETTQPSKQSNNKYM ASSYLTLTARAWERHSSYSCQVTHEGHTVEKSLSRADCS);
or a humanized variant thereof.

The epitope of the specific binding molecule may be within an amino acid sequence comprising residues 331 to 360 of SEQ ID NO: 1. Accordingly, the epitope may be within the amino acid sequence of SEQ ID NO: 321 (KPGGGQVEVKSEKLDFKDRVQSKIGSLDNI).

The epitope may be within an amino acid sequence comprising residues 331 to 360 of SEQ ID NO: 1. This epitope may be bound by the CDRs of the specific binding molecule referred to as “CE3” herein.

The epitope may comprise the amino acid sequence of SEQ ID NO: 321 (KPGGGQVEVKSEKLDFKDRVQSKIGSLDNI). The epitope may comprise an amino acid sequence having at least 70%, at least 75%, at least 80%, at least 90%, at least 95% or at least 99% identity to SEQ ID NO: 321 (KPGGGQVEVKSEKLDFKDRVQSKIGSLDNI).

The epitope may consist of the amino acid sequence of SEQ ID NO: 321 (KPGGGQVEVKSEKLDFKDRVQSKIGSLDNI). The epitope may consist of an amino acid sequence having at least 70%, at least 75%, at least 80%, at least 90%, at least 95% or at least 99% identity to SEQ ID NO: 321 (KPGGGQVEVKSEKLDFKDRVQSKIGSLDNI).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 17 (SNAVG);
VHCDR2 comprises the sequence set forth in SEQ ID NO: 20 (GCSSDGKCYYNSALKS);
VHCDR3 comprises the sequence set forth in SEQ ID NO: 23 (GYYPVYGYDYLGTIDY);
VLCDR1 comprises the sequence set forth in SEQ ID NO: 25 (SGSSSNVGRNDVA);
VLCDR2 comprises the sequence set forth in SEQ ID NO: 28 (GTTSRPS); and
VLCDR3 comprises the sequence set forth in SEQ ID NO: 31 (ASGDSSAINDI);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 331 to 360 of SEQ ID NO: 1. The specific binding molecule comprising CDRs having 100% identity to those given above is referred to as “CE3” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 17 (SNAVG);
VHCDR2 comprises the sequence set forth in SEQ ID NO: 20 (GCSSDGKCYYNSALKS);
VHCDR3 comprises the sequence set forth in SEQ ID NO: 23 (GYYPVYGYDYLGTIDY);
VLCDR1 comprises the sequence set forth in SEQ ID NO: 25 (SGSSSNVGRNDVA);
VLCDR2 comprises the sequence set forth in SEQ ID NO: 28 (GTTSRPS); and
VLCDR3 comprises the sequence set forth in SEQ ID NO: 31 (ASGDSSAINDI).

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 543 (QVRLQESGPSLVKPSQTLSVTCTVSGFSLI);
VHFR2 comprises the sequence set forth in SEQ ID NO: 544 (WVRQAPGKVPESLA);
VHFR3 comprises the sequence set forth in SEQ ID NO: n545 (RLDITRDTSKNQISLSLSSVTTDDAAVYYCTR);
VHFR4 comprises the sequence set forth in SEQ ID NO: 546 (WGPGLLVTVSS);
VLFR1 comprises the sequence set forth in SEQ ID NO: 547 (QAVLTQPSSVSGSLGQRVSITC);
VLFR2 comprises the sequence set forth in SEQ ID NO: 548 (WFQQLPGSGLRTIIY);
VLFR3 comprises the sequence set forth in SEQ ID NO: 549 (GIPDRFSGSKSGVTATLTIDSLQAEDEADYFC);
VLFR4 comprises the sequence set forth in SEQ ID NO: 550 (FGSGTRLTVLG);
or for each FR sequence, an amino acid sequence with
   (i) at least 50% identity thereto, and/or
   (ii) one, two, three, four or five amino acid substitutions relative thereto.

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 543 (QVRLQESGPSLVKPSQTLSVTCTVSGFSLI);
VHFR2 comprises the sequence set forth in SEQ ID NO: 544 (WVRQAPGKVPESLA);
VHFR3 comprises the sequence set forth in SEQ ID NO: 545 (RLDITRDTSKNQISLSLSSVTTDDAAVYYCTR);
VHFR4 comprises the sequence set forth in SEQ ID NO: 546 (WGPGLLVTVSS);
VLFR1 comprises the sequence set forth in SEQ ID NO: 547 (QAVLTQPSSVSGSLGQRVSITC);
VLFR2 comprises the sequence set forth in SEQ ID NO: 548 (WFQQLPGSGLRTIIY);
VLFR3 comprises the sequence set forth in SEQ ID NO: 549 (GIPDRFSGSKSGVTATLTIDSLQAEDEADYFC);
VLFR4 comprises the sequence set forth in SEQ ID NO: 550 (FGSGTRLTVLG);
or for each FR sequence, an amino acid sequence with
   (i) at least 50% identity thereto, and/or
   (ii) one, two, three, four or five amino acid substitutions relative thereto,
wherein the specific binding molecule binds to a polypeptide or protein molecule comprising an amino acid sequence comprising residues 331 to 360 of SEQ ID NO: 1. A specific binding molecule comprising FRs having 100% identity to those given above is referred to as “CE3” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 543 (QVRLQESGPSLVKPSQTLSVTCTVSGFSLI);
   VHFR2 comprises the sequence set forth in SEQ ID NO: 544 (WVRQAPGKVPESLA);
   VHFR3 comprises the sequence set forth in SEQ ID NO: 545 (RLDITRDTSKNQISLSLSSVTTDDAAVYYCTR);
   VHFR4 comprises the sequence set forth in SEQ ID NO: 546 (WGPGLLVTVSS);
   VLFR1 comprises the sequence set forth in SEQ ID NO: 547 (QAVLTQPSSVSGSLGQRVSITC);
   VLFR2 comprises the sequence set forth in SEQ ID NO: 548 (WFQQLPGSGLRTIIY);
   VLFR3 comprises the sequence set forth in SEQ ID NO: 549 (GIPDRFSGSKSGVTATLTIDSLQAEDEADYFC);
   VLFR4 comprises the sequence set forth in SEQ ID NO: 550 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 17 (SNAVG);
   VHCDR2 comprises the sequence set forth in SEQ ID NO: 20 (GCSSDGKCYYNSALKS);
   VHCDR3 comprises the sequence set forth in SEQ ID NO: 23 (GYYPVYGYDYLGTIDY);
   VLCDR1 comprises the sequence set forth in SEQ ID NO: 25 (SGSSSNVGRNDVA);
   VLCDR2 comprises the sequence set forth in SEQ ID NO: 28 (GTTSRPS); and
   VLCDR3 comprises the sequence set forth in SEQ ID NO: 31 (ASGDSSAINDI);
   or for each CDR sequence, an amino acid sequence with
      (i) at least 85% identity thereto, and/or
      (ii) one, two, or three amino acid substitutions relative thereto
   wherein the specific binding molecule binds to a polypeptide or protein molecule comprising an amino acid sequence comprising residues 331 to 360 of SEQ ID NO: 1. The specific binding molecule comprising FRs and CDRs having 100% identity to those given above is referred to as “CE3” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 543 (QVRLQESGPSLVKPSQTLSVTCTVSGFSLI);
   VHFR2 comprises the sequence set forth in SEQ ID NO: 544 (WVRQAPGKVPESLA);
   VHFR3 comprises the sequence set forth in SEQ ID NO: 545 (RLDITRDTSKNQISLSLSSVTTDDAAVYYCTR);
   VHFR4 comprises the sequence set forth in SEQ ID NO: 546 (WGPGLLVTVSS);
   VLFR1 comprises the sequence set forth in SEQ ID NO: 547 (QAVLTQPSSVSGSLGQRVSITC);
   VLFR2 comprises the sequence set forth in SEQ ID NO: 548 (WFQQLPGSGLRTIIY);
   VLFR3 comprises the sequence set forth in SEQ NO: 549 (GIPDRFSGSKSGVTATLTIDSLQAEDEADYFC);
   VLFR4 comprises the sequence set forth in SEQ ID NO: 550 (FGSGTRLTVLG);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 17 (SNAVG);
   VHCDR2 comprises the sequence set forth in SEQ ID NO: 20 (GCSSDGKCYYNSALKS);
   VHCDR3 comprises the sequence set forth in SEQ ID NO: 23 (GYYPVYGYDYLGTIDY);
   VLCDR1 comprises the sequence set forth in SEQ ID NO: 25 (SGSSSNVGRNDVA);
   VLCDR2 comprises the sequence set forth in SEQ ID NO: 28 (GTTSRPS); and
   VLCDR3 comprises the sequence set forth in SEQ ID NO: 31 (ASGDSSAINDI);
   wherein the specific binding molecule binds to a polypeptide or protein molecule comprising an amino acid sequence comprising residues 331 to 360 of SEQ ID NO: 1. The specific binding molecule comprising FRs and CDRs having 100% identity to those given above is referred to as “CE3” herein.

The specific binding molecule may comprise:
(a) A VH domain comprising the sequence set forth in SEQ ID NO: 551 (QVRLQESGPSLVKPSQTLSVTCTVSGFSLISNAVGWVRQAPGKVPESLAGCSSDGKCYY NSALKSRLDITRDTSKNQISLSLSSVTTDDAAVYYCTRGYYPVYGYDYLGTIDYWGPGLLV TVSS); and/or
(b) a VL domain comprising the sequence set forth in SEQ ID NO: 552 (QAVLTQPSSVSGSLGQRVSITCSGSSSNVGRNDVAWFQQLPGSGLRTIIYGTTSRPSGIP DRFSGSKSGVTATLTIDSLQAEDEADYFCASGDSSAINDIFGSGTRLTVLG);
or a humanized variant thereof.

The specific binding molecule may comprise:
(a) A heavy chain comprising the sequence set forth in SEQ ID NO: 553 (QVRLQESGPSLVKPSQTLSVTCTVSGFSLISNAVGWVRQAPGKVPESLAGCSSDGKCYY NSALKSRLDITRDTSKNQISLSLSSVTTDDAAVYYCTRGYYPVYGYDYLGTIDYWGPGLLV TVSSAKTTAPSVYPLAPVCGDTTGSSVTLGCLVKGYFPEPVTLTWNSGSLSSGVHTFPAV LQSDLYTLSSSVTVTSSTWPSQSITCNVAHPASSTKVDKKIEPRGPTIKPCPPCKCPAPNL LGGPSVFIFPPKIKDVLMISLSPIVTCVVVDVSEDDPDVQISWFVNNVEVHTAQTQTHRED YNSTLRVVSALPIQHQDWMSGKEFKCKVNNKDLPAPIERTISKPKGSVRAPQVYVLPPPE EEMTKKQVTLTCMVTDFMPEDIYVEWTNNGKTELNYKNTEPVLDSDGSYFMYSKLRVEK KNWVERNSYSCSVVHEGLHNHHTTKSFSRTPGK); and/or
(b) a light chain comprising the sequence set forth in SEQ ID NO: 554 (QAVLTQPSSVSGSLGQRVSITCSGSSSNVGRNDVAWFQQLPGSGLRTIIYGTTSRPSGIP DRFSGSKSGVTATLTIDSLQAEDEADYFCASGDSSAINDIFGSGTRLTVLGGQPKSSPSVT LFPPSSEELETNKATLVCTITDFYPGVVTVDWKVDGTPVTQGMETTQPSKQSNNKYMAS SYLTLTARAWERHSSYSCQVTHEGHTVEKSLSRADCS);
or a humanized variant thereof.

The epitope of the specific binding molecule may be within an amino acid sequence comprising residues 348 to 390 of SEQ ID NO: 1. Accordingly, the epitope may be within the amino acid sequence of SEQ ID NO: 322 (DRVQSKIGSLDNITHVPGGGNKKIETHKLTFRENAKAKTDHGA).

The epitope may be within an amino acid sequence comprising residues 348 to 390 of SEQ ID NO: 1. This epitope may be bound by the CDRs of the specific binding molecule referred to as “CA6” herein.

The epitope may comprise the amino acid sequence of SEQ ID NO: 322 (DRVQSKIGSLDNITHVPGGGNKKIETHKLTFRENAKAKTDHGA). The epitope may comprise an amino acid sequence having at least 70%, at least 75%, at least 80%, at least 90%, at least 95% or at least 99% identity to SEQ ID NO: 322 (DRVQSKIGSLDNITHVPGGGNKKIETHKLTFRENAKAKTDHGA).

The epitope may consist of the amino acid sequence of SEQ ID NO: 322 (DRVQSKIGSLDNITHVPGGGNKKIETHKLTFRENAKAKTDHGA). The epitope may consist of an amino acid sequence having at least 70%, at least 75%, at least 80%, at least 90%, at least 95% or at least 99% identity ID NO 322 to SEQ (DRVQSKIGSLDNITHVPGGGNKKIETHKLTFRENAKAKTDHGA).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 259 (SNGVG);
VHCDR2 comprises the sequence set forth in SEQ ID NO: 323 (DKSSGGKTYGNPALKS);
VHCDR3 comprises the sequence set forth in SEQ ID NO: 161 (CRDGGVTYGYDIDY);
VLCDR1 comprises the sequence set forth in SEQ ID NO: 324 (SGSRNNIGYGNHVG);
VLCDR2 comprises the sequence set forth in SEQ ID NO: 207 (DATTRAS); and
VLCDR3 comprises the sequence set forth in SEQ ID NO: 325 (ASFDRGSGGI);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 348 to 390 of SEQ ID NO: 1. The specific binding molecule comprising CDRs having 100% identity to those given above is referred to as “CA6” herein.

The epitope of the specific binding molecule may be within an amino acid sequence comprising residues 348 to 441 of SEQ ID NO: 1. Accordingly, the epitope may be within the amino acid sequence of SEQ ID NO 326 (DRVQSKIGSLDNITHVPGGGNKKIETHKLTFRENAKAKTDHGAEIVYKSPVVSGDTSPRHLSNVSS TGSIDMVDSPQLATLADEVSASLAKQGL).

The epitope may be within an amino acid sequence comprising residues 348 to 441 of SEQ ID NO: 1. This epitope may be bound by the CDRs of the specific binding molecule referred to as “CA11” herein.

The epitope may comprise the amino acid sequence of SEQ ID NO: 326 (DRVQSKIGSLDNITHVPGGGNKKIETHKLTFRENAKAKTDHGAEIVYKSPVVSGDTSPRHLSNVSS TGSIDMVDSPQLATLADEVSASLAKQGL). The epitope may comprise an amino acid sequence having at least 70%, at least 75%, at least 80%, at least 90%, at least 95% or at least 99% identity to SEQ ID NO: 326 (DRVQSKIGSLDNITHVPGGGNKKIETHKLTFRENAKAKTDHGAEIVYKSPVVSGDTSPRHLSNVSS TGSIDMVDSPQLATLADEVSASLAKQGL).

The epitope may consist of the amino acid sequence of SEQ ID NO: 326 (DRVQSKIGSLDNITHVPGGGNKKIETHKLTFRENAKAKTDHGAEIVYKSPVVSGDTSPRHLSNVSS TGSIDMVDSPQLATLADEVSASLAKQGL). The epitope may consist of an amino acid sequence having at least 70%, at least 75%, at least 80%, at least 90%, at least 95% or at least 99% identity to SEQ ID NO: 326 (DRVQSKIGSLDNITHVPGGGNKKIETHKLTFRENAKAKTDHGAEIVYKSPVVSGDTSPRHLSNVSS TGSIDMVDSPQLATLADEVSASLAKQGL).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 272 (SNTVA);
VHCDR2 comprises the sequence set forth in SEQ ID NO: 273 (EINSGGSTYYNPALKS);
VHCDR3 comprises the sequence set forth in SEQ ID NO: 274 (GARSTYAAY);
VLCDR1 comprises the sequence set forth in SEQ ID NO: 327 (SGSGSNIGAGNWVS);
VLCDR2 comprises the sequence set forth in SEQ ID NO: 328 (GATSRPS); and
VLCDR3 comprises the sequence set forth in SEQ ID NO: 329 (AAYDSGSSIV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 348 to 441 of SEQ ID NO: 1. The specific binding molecule comprising CDRs having 100% identity to those given above is referred to as “CA11” herein.

The epitope of the specific binding molecule may be within an amino acid sequence comprising residues 355 to 367 of SEQ ID NO: 1. Accordingly, the epitope may be within the amino acid sequence of SEQ ID NO: 330 (GSLDNITHVPGGG).

The epitope may be within an amino acid sequence comprising residues 355 to 367 of SEQ ID NO: 1. This epitope may be bound by the CDRs of the specific binding molecule referred to as “CA4” herein. Critical residues of the epitope may be residues 358 (D), 360 (I), 361 (T), 362 (H) and/or 364 (P) (numbering according to SEQ ID NO: 1). The epitope may comprise the amino acid sequence of SEQ ID NO: 331 (XXXDXITHXPXXX, wherein X is any amino acid). The epitope may comprise the amino acid sequence of SEQ ID NO: 330, wherein any one or more residue other than residue number 358 (D), 360 (I), 361 (T), 362 (H) and/or 364 (P) (numbering according to SEQ ID NO: 1) is replaced by a non-conservative amino acid substitution (numbering according to SEQ ID NO: 1). The epitope may comprise the amino acid sequence of SEQ ID NO: 330, wherein any one or more residue other than residue number 358 (D), 360 (I), 361 (T), 362 (H) and/or 364 (P) (numbering according to SEQ ID NO: 1) is replaced by a conservative amino acid substitution (numbering according to SEQ ID NO: 1). The epitope may comprise the amino acid sequence of SEQ ID NO: 330, wherein any one or more residue other than residue number 358 (D), 360 (I), 361 (T), 362 (H) and/or 364 (P) (numbering according to SEQ ID NO: 1) is replaced by a conservative amino acid substitution (numbering according to SEQ ID NO: 1) and any one or more residue other than residue number 358 (D), 360 (I), 361 (T), 362 (H) and/or 364 (P) (numbering according to SEQ ID NO: 1) is replaced by a non-conservative amino acid substitution (numbering according to SEQ ID NO: 1).

The epitope may comprise the amino acid sequence of SEQ ID NO: 330 (GSLDNITHVPGGG). The epitope may comprise an amino acid sequence having at least 70%, at least 75%, at least 80%, at least 90%, at least 95% or at least 99% identity to SEQ ID NO: 330 (GSLDNITHVPGGG).

The epitope may consist of the amino acid sequence of SEQ ID NO: 330 (GSLDNITHVPGGG). The epitope may consist of an amino acid sequence having at least 70%, at least 75%, at least 80%, at least 90%, at least 95% or at least 99% identity to SEQ ID NO: 330 (GSLDNITHVPGGG).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 83 (SYSVY);
VHCDR2 comprises the sequence set forth in SEQ ID NO: 84 (IMYASGRVDYNPALKS);
VHCDR3 comprises the sequence set forth in SEQ ID NO: 89 (GIEN);
VLCDR1 comprises the sequence set forth in SEQ ID NO: 91 (RTSQSVNNYLS);
VLCDR2 comprises the sequence set forth in SEQ ID NO: 95 (YATRLYT); and
VLCDR3 comprises the sequence set forth in SEQ ID NO: 97 (LQYDSTPLA);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 355 to 367 of SEQ ID NO: 1. The specific binding molecule comprising CDRs having 100% identity to those given above is referred to as “CA4” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 83 (SYSVY);
VHCDR2 comprises the sequence set forth in SEQ ID NO: 84 (IMYASGRVDYNPALKS);
VHCDR3 comprises the sequence set forth in SEQ ID NO: 89 (GIEN);
VLCDR1 comprises the sequence set forth in SEQ ID NO: 91 (RTSQSVNNYLS);
VLCDR2 comprises the sequence set forth in SEQ ID NO: 95 (YATRLYT); and
VLCDR3 comprises the sequence set forth in SEQ ID NO: 97 (LQYDSTPLA).

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 555 (QVQLQESGPSLVKPSQTLSLTCTVSGFSLT);
VHFR2 comprises the sequence set forth in SEQ ID NO: 556 (WVRQAPGQALEWIS);
VHFR3 comprises the sequence set forth in SEQ ID NO: 557 (RLSITRDTSKSQFSLSLSSVTTEDTAVYYCTR);
VHFR4 comprises the sequence set forth in SEQ ID NO: 558 (WGPGLLVTVSS);
VLFR1 comprises the sequence set forth in SEQ ID NO: 559 (DIQVTQSPSSLSASLTERVSITC);
VLFR2 comprises the sequence set forth in SEQ ID NO: 560 (WYQQKPGQAPKLLIY);
VLFR3 comprises the sequence set forth in SEQ ID NO: 561 (DVPSRFSGSGSGTDYTLTITSLEADDTATYYC);
VLFR4 comprises the sequence set forth in SEQ ID NO: 562 (FGGGTNVEIK);
or for each FR sequence, an amino acid sequence with
   (i) at least 50% identity thereto, and/or
   (ii) one, two, three, four or five amino acid substitutions relative thereto.

The specific binding molecule may comprise framework regions (FRs) VHFR1, VHFR2, VHFR3, VHFR4, VLFR1, VLFR2, VLFR3 and VLFR4, wherein each of said FRs comprises an amino acid sequence as follows:
VHFR1 comprises the sequence set forth in SEQ ID NO: 555 (QVQLQESGPSLVKPSQTLSLTCTVSGFSLT);
VHFR2 comprises the sequence set forth in SEQ ID NO: 556 (WVRQAPGQALEWIS);
VHFR3 comprises the sequence set forth in SEQ ID NO: 557 (RLSITRDTSKSQFSLSLSSVTTEDTAVYYCTR);
VHFR4 comprises the sequence set forth in SEQ ID NO: 558 (WGPGLLVTVSS);
VLFR1 comprises the sequence set forth in SEQ ID NO: 559 (DIQVTQSPSSLSASLTERVSITC);
VLFR2 comprises the sequence set forth in SEQ ID NO: 560 (WYQQKPGQAPKLLIY);
VLFR3 comprises the sequence set forth in SEQ ID NO: 561 (DVPSRFSGSGSGTDYTLTITSLEADDTATYYC);
VLFR4 comprises the sequence set forth in SEQ ID NO: 562 (FGGGTNVEIK);
or for each FR sequence, an amino acid sequence with
   (i) at least 50% identity thereto, and/or
   (ii) one, two, three, four or five amino acid substitutions relative thereto,
wherein the specific binding molecule binds to a polypeptide or protein molecule comprising an amino acid sequence comprising residues 355 to 367 of SEQ ID NO: 1. A specific binding molecule comprising FRs having 100% identity to those given above is referred to as “CA4” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 555 (QVQLQESGPSLVKPSQTLSLTCTVSGFSLT);
   VHFR2 comprises the sequence set forth in SEQ ID NO: 556 (WVRQAPGQALEWIS);
   VHFR3 comprises the sequence set forth in SEQ ID NO: 557 (RLSITRDTSKSQFSLSLSSVTTEDTAVYYCTR);
   VHFR4 comprises the sequence set forth in SEQ ID NO: 558 (WGPGLLVTVSS);
   VLFR1 comprises the sequence set forth in SEQ ID NO: 559 (DIQVTQSPSSLSASLTERVSITC);
   VLFR2 comprises the sequence set forth in SEQ ID NO: 560 (WYQQKPGQAPKLLIY);
   VLFR3 comprises the sequence set forth in SEQ ID NO: 561 (DVPSRFSGSGSGTDYTLTITSLEADDTATYYC);
   VLFR4 comprises the sequence set forth in SEQ ID NO: 562 (FGGGTNVEIK);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 83 (SYSVY);
   VHCDR2 comprises the sequence set forth in SEQ ID NO: 84 (IMYASGRVDYNPALKS);
   VHCDR3 comprises the sequence set forth in SEQ ID NO: 89 (GIEN);
   VLCDR1 comprises the sequence set forth in SEQ ID NO: 91 (RTSQSVNNYLS);
   VLCDR2 comprises the sequence set forth in SEQ ID NO: 95 (YATRLYT); and
   VLCDR3 comprises the sequence set forth in SEQ ID NO: 97 (LQYDSTPLA);
   or for each CDR sequence, an amino acid sequence with
      (i) at least 85% identity thereto, and/or
      (ii) one, two, or three amino acid substitutions relative thereto
   wherein the specific binding molecule binds to a polypeptide or protein molecule comprising an amino acid sequence comprising residues 355 to 367 of SEQ ID NO: 1. The specific binding molecule comprising FRs and CDRs having 100% identity to those given above is referred to as “CA4” herein.

The specific binding molecule may comprise:
(a) framework regions (FRs) VHFR1, VHFR2, VHFR3, VHFR4, VLFR1, VLFR2, VLFR3 and VLFR4, wherein each of said FRs comprises an amino acid sequence as follows:
   VHFR1 comprises the sequence set forth in SEQ ID NO: 555 (QVQLQESGPSLVKPSQTLSLTCTVSGFSLT);
   VHFR2 comprises the sequence set forth in SEQ ID NO: 556 (WVRQAPGQALEWIS);
   VHFR3 comprises the sequence set forth in SEQ ID NO: 557 (RLSITRDTSKSQFSLSLSSVTTEDTAVYYCTR);
   VHFR4 comprises the sequence set forth in SEQ ID NO: 558 (WGPGLLVTVSS);
   VLFR1 comprises the sequence set forth in SEQ ID NO: 559 (DIQVTQSPSSLSASLTERVSITC);
   VLFR2 comprises the sequence set forth in SEQ ID NO: 560 (WYQQKPGQAPKLLIY);
   VLFR3 comprises the sequence set forth in SEQ ID NO: 561 (DVPSRFSGSGSGTDYTLTITSLEADDTATYYC);
   VLFR4 comprises the sequence set forth in SEQ ID NO: 562 (FGGGTNVEIK);
   or for each FR sequence, an amino acid sequence with
      (i) at least 50% identity thereto, and/or
      (ii) one, two, three, four or five amino acid substitutions relative thereto; and
(b) the CDRs VHCDR1, VHCDR2, VHCDR3, VLCDR1, VLCDR2 and VLCDR3, wherein each of said CDRs comprises an amino acid sequence as follows:
   VHCDR1 comprises the sequence set forth in SEQ ID NO: 83 (SYSVY);
   VHCDR2 comprises the sequence set forth in SEQ ID NO: 84 (IMYASGRVDYNPALKS);
   VHCDR3 comprises the sequence set forth in SEQ ID NO: 89 (GIEN);
   VLCDR1 comprises the sequence set forth in SEQ ID NO: 91 (RTSQSVNNYLS);
   VLCDR2 comprises the sequence set forth in SEQ ID NO: 95 (YATRLYT); and
   VLCDR3 comprises the sequence set forth in SEQ ID NO: 97 (LQYDSTPLA);
   wherein the specific binding molecule binds to a polypeptide or protein molecule comprising an amino acid sequence comprising residues 355 to 367 of SEQ ID NO: 1. The specific binding molecule comprising FRs and CDRs having 100% identity to those given above is referred to as “CA4” herein.

The specific binding molecule may comprise:
(a) A VH domain comprising the sequence set forth in SEQ ID NO: 563 (QVQLQESGPSLVKPSQTLSLTCTVSGFSLTSYSVYWVRQAPGQALEWISIMYASGRVDY NPALKSRLSITRDTSKSQFSLSLSSVTTEDTAVYYCTRGIENWGPGLLVTVSS); and/or
(b) a VL domain comprising the sequence set forth in SEQ ID NO: 564 (DIQVTQSPSSLSASLTERVSITCRTSQSVNNYLSWYQQKPGQAPKLLIYYATRLYTDVPS RFSGSGSGTDYTLTITSLEADDTATYYCLQYDSTPLAFGGGTNVEIK);
or a humanized variant thereof.

The specific binding molecule may comprise:
(a) A heavy chain comprising the sequence set forth in SEQ ID NO: 565 (QVQLQESGPSLVKPSQTLSLTCTVSGFSLTSYSVYWVRQAPGQALEWISIMYASGRVDY NPALKSRLSITRDTSKSQFSLSLSSVTTEDTAVYYCTRGIENWGPGLLVTVSSAKTTAPSV YPLAPVCGDTTGSSVTLGCLVKGYFPEPVTLTWNSGSLSSGVHTFPAVLQSDLYTLSSSV TVTSSTWPSQSITCNVAHPASSTKVDKKIEPRGPTIKPCPPCKCPAPNLLGGPSVFIFPPKI KDVLMISLSPIVTCVVVDVSEDDPDVQISWFVNNVEVHTAQTQTHREDYNSTLRVVSALPI QHQDWMSGKEFKCKVNNKDLPAPIERTISKPKGSVRAPQVYVLPPPEEEMTKKQVTLTC MVTDFMPEDIYVEWTNNGKTELNYKNTEPVLDSDGSYFMYSKLRVEKKNWVERNSYSCS VVHEGLHNHHTTKSFSRTPGK); and/or
(b) a light chain comprising the sequence set forth in SEQ ID NO: 566 (DIQVTQSPSSLSASLTERVSITCRTSQSVNNYLSWYQQKPGQAPKLLIYYATRLYTDVPS RFSGSGSGTDYTLTITSLEADDTATYYCLQYDSTPLAFGGGTNVEIKGQPKSSPSVTLFPP SSEELETNKATLVCTITDFYPGVVTVDWKVDGTPVTQGMETTQPSKQSNNKYMASSYLTL TARAWERHSSYSCQVTHEGHTVEKSLSRADCS);
or a humanized variant thereof.

The epitope of the specific binding molecule may be within an amino acid sequence comprising residues 359 to 391 of SEQ ID NO: 1. Accordingly, the epitope may be within the amino acid sequence of SEQ ID NO: 332 (NITHVPGGGNKKIETHKLTFRENAKAKTDHGAE).

The epitope may be within an amino acid sequence comprising residues 359 to 391 of SEQ ID NO: 1. This epitope may be bound by the CDRs of the specific binding molecule referred to as “CB2” herein.

The epitope may comprise the amino acid sequence of SEQ ID NO: 332 (NITHVPGGGNKKIETHKLTFRENAKAKTDHGAE). The epitope may comprise an amino acid sequence having at least 70%, at least 75%, at least 80%, at least 90%, at least 95% or at least 99% identity to SEQ ID NO: 332 (NITHVPGGGNKKIETHKLTFRENAKAKTDHGAE).

The epitope may consist of the amino acid sequence of SEQ ID NO: 332 (NITHVPGGGNKKIETHKLTFRENAKAKTDHGAE). The epitope may consist of an amino acid sequence having at least 70%, at least 75%, at least 80%, at least 90%, at least 95% or at least 99% identity to SEQ ID NO: 332 (NITHVPGGGNKKIETHKLTFRENAKAKTDHGAE).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 45 (TNSVG);
VHCDR2 comprises the sequence set forth in SEQ ID NO: 53 (GIDTDGEEGFNPVLKS);
VHCDR3 comprises the sequence set forth in SEQ ID NO: 55 (SYRTDGLAYGYVQAIDY);
VLCDR1 comprises the sequence set forth in SEQ ID NO: 68 (SGSYIGSSGVG);
VLCDR2 comprises the sequence set forth in SEQ ID NO: 70 (ASDGRPS); and
VLCDR3 comprises the sequence set forth in SEQ ID NO: 79 (GSSDRTQYTGL);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 359 to 391 of SEQ ID NO: 1. The specific binding molecule comprising CDRs having 100% identity to those given above is referred to as “CB2” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 45 (TNSVG);
VHCDR2 comprises the sequence set forth in SEQ ID NO: 53 (GIDTDGEEGFNPVLKS);
VHCDR3 comprises the sequence set forth in SEQ ID NO: 55 (SYRTDGLAYGYVQAIDY);
VLCDR1 comprises the sequence set forth in SEQ ID NO: 68 (SGSYIGSSGVG);
VLCDR2 comprises the sequence set forth in SEQ ID NO: 70 (ASDGRPS); and
VLCDR3 comprises the sequence set forth in SEQ ID NO: 79 (GSSDRTQYTGL).

The epitope of the specific binding molecule may be within an amino acid sequence comprising residues 360 to 390 of SEQ ID NO: 1. Accordingly, the epitope may be within the amino acid sequence of SEQ ID NO: 333 (ITHVPGGGNKKIETHKLTFRENAKAKTDHGA).

The epitope may be within an amino acid sequence comprising residues 360 to 390 of SEQ ID NO: 1. This epitope may be bound by the CDRs of the specific binding molecule referred to as “CB3” herein.

The epitope may comprise the amino acid sequence of SEQ ID NO: 333 (ITHVPGGGNKKIETHKLTFRENAKAKTDHGA). The epitope may comprise an amino acid sequence having at least 70%, at least 75%, at least 80%, at least 90%, at least 95% or at least 99% identity to SEQ ID NO: 333 (ITHVPGGGNKKIETHKLTFRENAKAKTDHGA).

The epitope may consist of the amino acid sequence of SEQ ID NO: 333 (ITHVPGGGNKKIETHKLTFRENAKAKTDHGA). The epitope may consist of an amino acid sequence having at least 70%, at least 75%, at least 80%, at least 90%, at least 95% or at least 99% identity to SEQ ID NO: 333 (ITHVPGGGNKKIETHKLTFRENAKAKTDHGA).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 334 (SVAVN);
VHCDR2 comprises the sequence set forth in SEQ ID NO: 335 (GIISNGGTGYNPALKS);
VHCDR3 comprises the sequence set forth in SEQ ID NO: 336 (GVEWEGSMDY);
VLCDR1 comprises the sequence set forth in SEQ ID NO: 337 (SGSSSNVGAGSYVG);
VLCDR2 comprises the sequence set forth in SEQ ID NO: 338 (GATKRAS); and
VLCDR3 comprises the sequence set forth in SEQ ID NO: 339 (VSYQTDFTLV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 360 to 390 of SEQ ID NO: 1. The specific binding molecule comprising CDRs having 100% identity to those given above is referred to as “CB3” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 334 (SVAVN);
VHCDR2 comprises the sequence set forth in SEQ ID NO: 335 (GIISNGGTGYNPALKS);
VHCDR3 comprises the sequence set forth in SEQ ID NO: 336 (GVEWEGSMDY);
VLCDR1 comprises the sequence set forth in SEQ ID NO: 337 (SGSSSNVGAGSYVG);
VLCDR2 comprises the sequence set forth in SEQ ID NO: 338 (GATKRAS); and
VLCDR3 comprises the sequence set forth in SEQ ID NO: 339 (VSYQTDFTLV).

The epitope of the specific binding molecule may be within an amino acid sequence comprising residues 367 to 379 of SEQ ID NO: 1. Accordingly, the epitope may be within the amino acid sequence of SEQ ID NO: 33 (GNKKIETHKLTFR).

The epitope may be within an amino acid sequence comprising residues 367 to 379 of SEQ ID NO: 1. This epitope may be bound by the CDRs of the specific binding molecules referred to as “CA9” and “CA12” herein. Critical residues of the epitope may be residues 370 (K), and/or 374 (H) (numbering according to SEQ ID NO: 1). The epitope may comprise the amino acid sequence of SEQ ID NO: 34 (XXXKXXXHXXXXX, wherein X is any amino acid). The epitope may comprise the amino acid sequence of SEQ ID NO: 33, wherein any one or more residue other than residue number 370 (K), and/or 374 (H) is replaced by a non-conservative amino acid substitution (numbering according to SEQ ID NO: 1). The epitope may comprise the amino acid sequence of SEQ ID NO: 33, wherein any one or more residue other than residue number 370 (K), and/or 374 (H) is replaced by a conservative amino acid substitution (numbering according to SEQ ID NO: 1). The epitope may comprise the amino acid sequence of SEQ ID NO: 33, wherein any one or more residue other than residue number 370 (K), and/or 374 (H) is replaced by a conservative amino acid substitution (numbering according to SEQ ID NO: 1) and any one or more residue other than residue number 370 (K), and/or 374 (H) is replaced by a non-conservative amino acid substitution (numbering according to SEQ ID NO: 1).

The epitope may comprise the amino acid sequence of SEQ ID NO: 33 (GNKKIETHKLTFR). The epitope may comprise an amino acid sequence having at least 70%, at least 75%, at least 80%, at least 90%, at least 95% or at least 99% identity to SEQ ID NO: 33 (GNKKIETHKLTFR).

The epitope may consist of the amino acid sequence of SEQ ID NO: 33 (GNKKIETHKLTFR). The epitope may consist of an amino acid sequence having at least 70%, at least 75%, at least 80%, at least 90%, at least 95% or at least 99% identity to SEQ ID NO: 33 (GNKKIETHKLTFR).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 58 (SYRSDGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS); and
VLCDR3 comprises the sequence set forth in SEQ ID NO: 74 (GSSDRTQYTGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 367 to 379 of SEQ ID NO: 1. The specific binding molecule comprising CDRs having 100% identity to those given above is referred to as “CA9” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 58 (SYRSDGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS); and
VLCDR3 comprises the sequence set forth in SEQ ID NO: 74 (GSSDRTQYTGV).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 44 (SYYVG);
VHCDR2 comprises the sequence set forth in SEQ ID NO: 52 (NIYSTGRAFYNPALKS);
VHCDR3 comprises the sequence set forth in SEQ ID NO: 265 (GSYYHGGGNGMVDFFDY);
VLCDR1 comprises the sequence set forth in SEQ ID NO: 39 (SGSSSNVGYGNYVG);
VLCDR2 comprises the sequence set forth in SEQ ID NO: 72 (AATSRAS); and
VLCDR3 comprises the sequence set forth in SEQ ID NO: 78 (SSYQRGNTGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 367 to 379 of SEQ ID NO: 1. The specific binding molecule comprising CDRs having 100% identity to those given above is referred to as “CA12” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 44 (SYYVG);
VHCDR2 comprises the sequence set forth in SEQ ID NO: 52 (NIYSTGRAFYNPALKS);
VHCDR3 comprises the sequence set forth in SEQ ID NO: 265 (GSYYHGGGNGMVDFFDY);
VLCDR1 comprises the sequence set forth in SEQ ID NO: 39 (SGSSSNVGYGNYVG);
VLCDR2 comprises the sequence set forth in SEQ ID NO: 72 (AATSRAS); and
VLCDR3 comprises the sequence set forth in SEQ ID NO: 78 (SSYQRGNTGV).

The epitope of the specific binding molecule may be within an amino acid sequence comprising residues 49 to 113 of SEQ ID NO: 1. Accordingly, the epitope may be within the amino acid sequence of SEQ ID NO: 340 (QTPTEDGSEEPGSETSDAKSTPTAEDVTAPLVDEGAPGKQAAAQPHTEIPEGTTAEEAGIGDTPS).

The epitope may be within an amino acid sequence comprising residues 49 to 113 of SEQ ID NO: 1. This epitope may be bound by the CDRs of the specific binding molecule referred to as “CB5” herein.

The epitope may comprise the amino acid sequence of SEQ ID NO: 340 (QTPTEDGSEEPGSETSDAKSTPTAEDVTAPLVDEGAPGKQAAAQPHTEIPEGTTAEEAGIGDTPS). The epitope may comprise an amino acid sequence having at least 70%, at least 75%, at least 80%, at least 90%, at least 95% or at least 99% identity to SEQ ID NO: 340 (QTPTEDGSEEPGSETSDAKSTPTAEDVTAPLVDEGAPGKQAAAQPHTEIPEGTTAEEAGIGDTPS).

The epitope may consist of the amino acid sequence of SEQ ID NO: 340 (QTPTEDGSEEPGSETSDAKSTPTAEDVTAPLVDEGAPGKQAAAQPHTEIPEGTTAEEAGIGDTPS). The epitope may consist of an amino acid sequence having at least 70%, at least 75%, at least 80%, at least 90%, at least 95% or at least 99% identity to SEQ ID NO: 340 (QTPTEDGSEEPGSETSDAKSTPTAEDVTAPLVDEGAPGKQAAAQPHTEIPEGTTAEEAGIGDTPS).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 259 (SNGVG);
VHCDR2 comprises the sequence set forth in SEQ ID NO: 341 (DITSGGRTYGNLALKS);
VHCDR3 comprises the sequence set forth in SEQ ID NO: 160 (CRDGGVSYGYDSDY);
VLCDR1 comprises the sequence set forth in SEQ ID NO: 342 (SGSSSNVGSGDHVN);
VLCDR2 comprises the sequence set forth in SEQ ID NO: 343 (RTTNRAS); and
VLCDR3 comprises the sequence set forth in SEQ ID NO: 344 (ASHDNNSGGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 49 to 113 of SEQ ID NO: 1. The specific binding molecule comprising CDRs having 100% identity to those given above is referred to as “CB5” herein.

The epitope of the specific binding molecule may be within an amino acid sequence comprising residues 49 to 155 of SEQ ID NO: 1. Accordingly, the epitope may be within the amino acid sequence of SEQ ID NO: 345 (QTPTEDGSEEPGSETSDAKSTPTAEDVTAPLVDEGAPGKQAAAQPHTEIPEGTTAEEAGIGDTPS LEDEAAGHVTQARMVSKSKDGTGSDDKKAKGADGKTKIATPR).

The epitope may be within an amino acid sequence comprising residues 49 to 155 of SEQ ID NO: 1. This epitope may be bound by the CDRs of the specific binding molecules referred to as “CC4” and “CD1” herein.

The epitope may comprise the amino acid sequence of SEQ ID NO: 345 (QTPTEDGSEEPGSETSDAKSTPTAEDVTAPLVDEGAPGKQAAAQPHTEIPEGTTAEEAGIGDTPS LEDEAAGHVTQARMVSKSKDGTGSDDKKAKGADGKTKIATPR). The epitope may comprise an amino acid sequence having at least 70%, at least 75%, at least 80%, at least 90%, at least 95% or at least 99% identity to SEQ ID NO: 345 (QTPTEDGSEEPGSETSDAKSTPTAEDVTAPLVDEGAPGKQAAAQPHTEIPEGTTAEEAGIGDTPS LEDEAAGHVTQARMVSKSKDGTGSDDKKAKGADGKTKIATPR).

The epitope may consist of the amino acid sequence of SEQ ID NO: 345 (QTPTEDGSEEPGSETSDAKSTPTAEDVTAPLVDEGAPGKQAAAQPHTEIPEGTTAEEAGIGDTPS LEDEAAGHVTQARMVSKSKDGTGSDDKKAKGADGKTKIATPR). The epitope may consist of an amino acid sequence having at least 70%, at least 75%, at least 80%, at least 90%, at least 95% or at least 99% identity to SEQ ID NO: 345 (QTPTEDGSEEPGSETSDAKSTPTAEDVTAPLVDEGAPGKQAAAQPHTEIPEGTTAEEAGIGDTPS LEDEAAGHVTQARMVSKSKDGTGSDDKKAKGADGKTKIATPR).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 259 (SNGVG);
VHCDR2 comprises the sequence set forth in SEQ ID NO: 158 (DIASSGKAYSNPALKS);
VHCDR3 comprises the sequence set forth in SEQ ID NO: 161 (CRDGGVTYGYDIDY);
VLCDR1 comprises the sequence in set forth SEQ ID NO: 346 (SGSSSNVGYTNLGYSNLVT);
VLCDR2 comprises the sequence set forth in SEQ ID NO: 170 (GATNRAS); and
VLCDR3 comprises the sequence set forth in SEQ ID NO: 347 (ASYDSSNGGI);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 49 to 155 of SEQ ID NO: 1. The specific binding molecule comprising CDRs having 100% identity to those given above is referred to as “CC4” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 259 (SNGVG);
VHCDR2 comprises the sequence set forth in SEQ ID NO: 182 (DKSSAGKTYGNPALKS);
VHCDR3 comprises the sequence set forth in SEQ ID NO: 286 (CRDGGVSYGYDVDY);
VLCDR1 comprises the sequence set forth in SEQ ID NO: 290 (SGSSSNVGYGTYVS);
VLCDR2 comprises the sequence set forth in SEQ ID NO: 188 (GTTTRAS); and
VLCDR3 comprises the sequence set forth in SEQ ID NO: 291 (ASYDTGSGGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 49 to 155 of SEQ ID NO: 1. The specific binding molecule comprising CDRs having 100% identity to those given above is referred to as “CD1” herein.

The epitope of the specific binding molecule may be within an amino acid sequence comprising residues 49 to 238 of SEQ ID NO: 1. Accordingly, the epitope may be within the amino acid sequence of SEQ ID NO: 348 (QTPTEDGSEEPGSETSDAKSTPTAEDVTAPLVDEGAPGKQAAAQPHTEIPEGTTAEEAGIGDTPS LEDEAAGHVTQARMVSKSKDGTGSDDKKAKGADGKTKIATPRGAAPPGQKGQANATRIPAKTPP APKTPPSSGEPPKSGDRSGYSSPGSPGTPGSRSRTPSLPTPPTREPKKVAVVRTPPKSPSS).

The epitope may be within an amino acid sequence comprising residues 49 to 238 of SEQ ID NO: 1. This epitope may be bound by the CDRs of the specific binding molecule referred to as “CC5” herein.

The epitope may comprise the amino acid sequence of SEQ ID NO: 348 (QTPTEDGSEEPGSETSDAKSTPTAEDVTAPLVDEGAPGKQAAAQPHTEIPEGTTAEEAGIGDTPS LEDEAAGHVTQARMVSKSKDGTGSDDKKAKGADGKTKIATPRGAAPPGQKGQANATRIPAKTPP APKTPPSSGEPPKSGDRSGYSSPGSPGTPGSRSRTPSLPTPPTREPKKVAVVRTPPKSPSS). The epitope may comprise an amino acid sequence having at least 70%, at least 75%, at least 80%, at least 90%, at least 95% or at least 99% identity to SEQ ID NO: 348 (QTPTEDGSEEPGSETSDAKSTPTAEDVTAPLVDEGAPGKQAAAQPHTEIPEGTTAEEAGIGDTPS LEDEAAGHVTQARMVSKSKDGTGSDDKKAKGADGKTKIATPRGAAPPGQKGQANATRIPAKTPP APKTPPSSGEPPKSGDRSGYSSPGSPGTPGSRSRTPSLPTPPTREPKKVAVVRTPPKSPSS).

The epitope may consist of the amino acid sequence of SEQ ID NO: 348 (QTPTEDGSEEPGSETSDAKSTPTAEDVTAPLVDEGAPGKQAAAQPHTEIPEGTTAEEAGIGDTPS LEDEAAGHVTQARMVSKSKDGTGSDDKKAKGADGKTKIATPRGAAPPGQKGQANATRIPAKTPP APKTPPSSGEPPKSGDRSGYSSPGSPGTPGSRSRTPSLPTPPTREPKKVAVVRTPPKSPSS). The epitope may consist of an amino acid sequence having at least 70%, at least 75%, at least 80%, at least 90%, at 95% or least at least 99% identity to SEQ ID NO: 348 (QTPTEDGSEEPGSETSDAKSTPTAEDVTAPLVDEGAPGKQAAAQPHTEIPEGTTAEEAGIGDTPS LEDEAAGHVTQARMVSKSKDGTGSDDKKAKGADGKTKIATPRGAAPPGQKGQANATRIPAKTPP APKTPPSSGEPPKSGDRSGYSSPGSPGTPGSRSRTPSLPTPPTREPKKVAVVRTPPKSPSS).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 259 (SNGVG);
VHCDR2 comprises the sequence set forth in SEQ ID NO: 349 (DISSVGKKYASPALKS);
VHCDR3 comprises the sequence set forth in SEQ ID NO: 161 (CRDGGVTYGYDIDY);
VLCDR1 comprises the sequence set forth in SEQ ID NO: 39 (SGSSSNVGYGNYVG);
VLCDR2 comprises the sequence set forth in SEQ ID NO: 141 (GATSRAS); and
VLCDR3 comprises the sequence set forth in SEQ ID NO: 350 (ASYDSSNGGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 49 to 238 of SEQ ID NO: 1. The specific binding molecule comprising CDRs having 100% identity to those given above is referred to as “CC5” herein.

The epitope of the specific binding molecule may be within an amino acid sequence comprising residues 373 to 385 of SEQ ID NO: 1. Accordingly, the epitope may be within the amino acid sequence of SEQ ID NO: 351 (THKLTFRENAKAK).

The epitope may be within an amino acid sequence comprising residues 373 to 385 of SEQ ID NO: 1. This epitope may be bound by the CDRs of the specific binding molecule referred to as “MD9” or “MoD9” herein.

The epitope may comprise the amino acid sequence of SEQ ID NO: 351 (THKLTFRENAKAK). The epitope may comprise an amino acid sequence having at least 70%, at least 75%, at least 80%, at least 90%, at least 95% or at least 99% identity to SEQ ID NO: 351 (THKLTFRENAKAK).

The epitope may consist of the amino acid sequence of SEQ ID NO: 351 (THKLTFRENAKAK). The epitope may consist of an amino acid sequence having at least 70%, at least 75%, at least 80%, at least 90%, at least 95% or at least 99% identity to SEQ ID NO: 351 (THKLTFRENAKAK).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 102 (RESIA);
VHCDR2 comprises the sequence set forth in SEQ ID NO: 103 (GVGIDGTSYYSPALKS);
VHCDR3 comprises the sequence set forth in SEQ ID NO: 104 (NYIDFEY);
VLCDR1 comprises the sequence set forth in SEQ ID NO: 352 (SGSSSNVGIYDVS);
VLCDR2 comprises the sequence set forth in SEQ ID NO: 353 (GTNNRPS); and
VLCDR3 comprises the sequence set forth in SEQ ID NO: 354 (AAGDSSTIAV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 373 to 385 of SEQ ID NO: 1. The specific binding molecule comprising CDRs having 100% identity to those given above is referred to as “MD9” or “MoD9” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 102 (RESIA);
VHCDR2 comprises the sequence set forth in SEQ ID NO: 103 (GVGIDGTSYYSPALKS);
VHCDR3 comprises the sequence set forth in SEQ ID NO: 104 (NYIDFEY);
VLCDR1 comprises the sequence set forth in SEQ ID NO: 352 (SGSSSNVGIYDVS);
VLCDR2 comprises the sequence set forth in SEQ ID NO: 353 (GTNNRPS); and
VLCDR3 comprises the sequence set forth in SEQ ID NO: 354 (AAGDSSTIAV).

The epitope of the specific binding molecule may be within an amino acid sequence comprising residues 275 to 305 and/or residues 337 to 368 of SEQ ID NO: 1. Accordingly, the epitope may be within the amino acid sequence of SEQ ID NO: 355 (VQIINKKLDLSNVQSKCGSKDNIKHVPGGGS) and/or SEQ ID NO: 356 (VEVKSEKLDFKDRVQSKIGSLDNITHVPGGGN).

The epitope may be within an amino acid sequence comprising residues 275 to 305 and/or residues 337 to 368 of SEQ ID NO: 1. This epitope may be bound by the CDRs of the specific binding molecule referred to as “NS1G7” herein.

The epitope may comprise the amino acid sequence of SEQ ID NO: 355 (VQIINKKLDLSNVQSKCGSKDNIKHVPGGGS) and/or SEQ ID NO: 356 (VEVKSEKLDFKDRVQSKIGSLDNITHVPGGGN). The epitope may comprise an amino acid sequence having at least 70%, at least 75%, at least 80%, at least 90%, at least 95% or at least 99% identity to SEQ ID NO: 355 (VQIINKKLDLSNVQSKCGSKDNIKHVPGGGS) and/or SEQ ID NO: 356 (VEVKSEKLDFKDRVQSKIGSLDNITHVPGGGN).

The epitope may consist of the amino acid sequence of SEQ ID NO: 355 (VQIINKKLDLSNVQSKCGSKDNIKHVPGGGS) and/or SEQ ID NO: 356 (VEVKSEKLDFKDRVQSKIGSLDNITHVPGGGN). The epitope may consist of an amino acid sequence having at least 70%, at least 75%, at least 80%, at least 90%, at least 95% or at least 99% identity to SEQ ID NO: 355 (VQIINKKLDLSNVQSKCGSKDNIKHVPGGGS) and/or SEQ ID NO: 356 (VEVKSEKLDFKDRVQSKIGSLDNITHVPGGGN).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 357 (SYGVG);
VHCDR2 comprises the sequence set forth in SEQ ID NO: 358 (SISSGGTTFYNPALKS);
VHCDR3 set comprises the sequence forth in SEQ ID NO: 359 (DVHIYYNDYGAAYGDRDY);
VLCDR1 comprises the sequence set forth in SEQ ID NO: 360 (SGSSSNIGGGNYVS);
VLCDR2 comprises the sequence set forth in SEQ ID NO: 361 (GTTSRAS); and
VLCDR3 comprises the sequence set forth in SEQ ID NO: 362 (ASYDTNSGSV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 275 to 305 and/or residues 337 to 368 of SEQ ID NO: 1. The specific binding molecule comprising CDRs having 100% identity to those given above is referred to as “NS1G7” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 357 (SYGVG);
VHCDR2 comprises the sequence set forth in SEQ ID NO: 358 (SISSGGTTFYNPALKS);
VHCDR3 comprises the sequence set forth in SEQ ID NO: 359 (DVHIYYNDYGAAYGDRDY);
VLCDR1 comprises the sequence set forth in SEQ ID NO: 360 (SGSSSNIGGGNYVS);
VLCDR2 comprises the sequence set forth in SEQ ID NO: 361 (GTTSRAS); and
VLCDR3 comprises the sequence set forth in SEQ ID NO: 362 (ASYDTNSGSV).

The specific binding molecule may comprise the CDR sequences of a clone set out in Table 10 below.

TABLE 10

| Clone | VH | | | VL | | | |
|---|---|---|---|---|---|---|---|
| name | CDR1 | CDR2 | CDR3 | CDR1 | CDR2 | CDR3 | Epitope |
| CB11 | SNAVI | LIDVD | DYGSW | SGSNI | DNNNR | GGYAG | 113-238 |
| | (SEQ | GDAAY | GYVSD | GSNDV | PS | SSSNF | |
| | ID | DPALK | IDY | G | (SEQ | L | |
| | NO: | S | (SEQ | (SEQ | ID | (SEQ | |
| | 198) | (SEQ | ID | ID | NO: | ID | |
| | | ID | NO: | NO: | 257) | NO: | |
| | | NO: | 202) | 256) | | 258) | |
| | | 200) | | | | | |
| CA2 | SNGVG | DISSV | CRDGG | SGSSG | GATNL | ASYDS | 1-155 |
| | (SEQ | GKKYA | VTYGY | NVGYG | AS | SSGGV | |
| | ID | NPALK | DIDY | DYVS | (SEQ | (SEQ | |
| | NO: | S | (SEQ | (SEQ | ID | ID | |
| | 259) | (SEQ | ID | ID | NO: | NO: | |
| | | ID | NO: | NO: | 169) | 173) | |
| | | NO: | 161) | 165) | | | |
| | | 157) | | | | | |
| CB6 | SNGVG | DISSV | CRDGG | SGSSS | GAVTR | ASYDS | 1-238 |
| | (SEQ | GKKYA | VTYGY | NIGTG | AS | TSGGV | |
| | ID | NPALK | DIDY | NYVG | (SEQ | (SEQ | |
| | NO: | S | (SEQ | (SEQ | ID | ID | |
| | 259) | (SEQ | ID | ID | NO: | NO: | |
| | | ID | NO: | NO: | 262) | 263) | |
| | | NO: | 161) | 261) | | | |
| | | 157) | | | | | |
| CA7 | SYYVG | NIYST | GSYYH | SGSSS | AATSR | SSYQR | 1-319 |
| | (SEQ | GRAFY | GGGNG | NVGYG | AS | GNTGV | |
| | ID | NPALK | MVDFF | NYVG | (SEQ | (SEQ | |
| | NO: | S | DY | (SEQ | ID | ID | |
| | 44) | (SEQ | (SEQ | ID | NO: | NO: | |
| | | ID | ID | NO: | 72) | 78) | |
| | | NO: | NO: | 39) | | | |
| | | 52) | 265) | | | | |
| CA8 | SNAVV | AIDKD | DPSGW | SGTYI | GTSSR | ATYES | 1-319 |
| | (SEQ | GDTIY | GYPDV | GSSDV | PS | SYHNS | |
| | ID | NPALK | DY | G | (SEQ | V | |
| | NO: | S | (SEQ | (SEQ | ID | (SEQ | |
| | 266) | (SEQ | ID | ID | NO: | ID | |
| | | ID | | NO: | 270) | NO: | |
| | | NO: | NO: | 269) | | 271) | |
| | | 267) | 268) | | | | |
| CB10 | SNTVA | EINSG | GARST | SGSSS | HISNR | AAYDS | 1-319 |
| | (SEQ | GSTYY | YAAY | DVGYS | AS | SNNVW | |
| | ID | NPALK | (SEQ | TWVY | (SEQ | I | |
| | NO: | S | ID | (SEQ | ID | (SEQ | |
| | 272) | (SEQ | NO: | ID | NO: | ID | |
| | | ID | 274) | NO: | 276) | NO: | |
| | | NO: | | 275) | | 277) | |
| | | 273) | | | | | |
| CB7 | NYRVG | NIRSG | DSSGD | SGSSS | GATSR | ASYDS | 13-25 |
| | (SEQ | GTTWY | LYAYD | NVGYG | AS | TSGGV | |
| | ID | NPALK | Y | NYMA | (SEQ | (SEQ | |
| | NO: | S | (SEQ | (SEQ | ID | ID | |
| | 279) | (SEQ | ID | ID | NO: | NO: | |
| | | ID | NO: | NO: | 141) | 263) | |
| | | NO: | 281) | 282) | | | |
| | | 280) | | | | | |
| CC7 | SNAVI | LIDVD | DYGSW | SGSYI | DNNDR | ASYDT | 145-157 |
| | (SEQ | GDAAY | GYVSD | TGSSV | PS | SNIGL | |
| | ID | DPALK | IDY | G | (SEQ | (SEQ | |
| | NO: | S | (SEQ | (SEQ | ID | ID | |
| | 198) | (SEQ | ID | ID | NO: | NO: | |
| | | ID | NO: | NO: | 284) | 285) | |
| | | NO: | 202) | 292) | | | |
| | | 200) | | | | | |
| CB12 | SNGVG | DKSSA | CRDGG | SGSSS | DTTSR | ASVDK | 155-227 |
| | (SEQ | GKTYG | VSYGY | NVGGD | PS | TTGGV | |
| | ID | NPALK | DVDY | YVG | (SEQ | (SEQ | |
| | NO: | S | (SEQ | (SEQ | ID | ID | |
| | 259) | (SEQ | ID | ID | NO: | NO: | |
| | | ID | NO: | NO: | 288) | 289) | |
| | | NO: | 286) | 287) | | | |
| | | 182) | | | | | |
| CC3 | SNGVG | DKSSA | CRDGG | SGSSS | GTTTR | ASYDT | 155-227 |
| | (SEQ | GKTYG | VSYGY | NVGYG | AS | GSGGV | |
| | ID | NPALK | DVDY | TYVS | (SEQ | (SEQ | |
| | NO: | S | (SEQ | (SEQ | ID | ID | |
| | 259) | (SEQ | ID | ID | NO: | NO: | |
| | | ID | NO: | NO: | 188) | 291) | |
| | | NO: | 286) | 290) | | | |
| | | 182) | | | | | |
| CA1 | SNAVI | DIRAD | PGNYY | SGSSS | DTTSR | AAMDS | 155-238 |
| | (SEQ | GATNY | YGAGR | NIGGG | PS | SSLIG | |
| | ID | NAALK | DVARL | NAVG | (SEQ | V | |
| | NO: | S | AD | (SEQ | ID | (SEQ | |
| | 198) | (SEQ | (SEQ | ID | NO: | ID | |
| | | ID | ID | NO: | 288) | NO: | |
| | | NO: | NO: | 298) | | 299) | |
| | | 296) | 297) | | | | |
| CA3 | SNGVG | DKSSA | CRDGG | SGSSG | GATRR | ASYDS | 186-263 |
| | (SEQ | GKTYG | VTYGY | NIGYD | SS | SGGGV | |
| | ID | NPALK | DVDY | DYVS | (SEQ | (SEQ | |
| | NO: | S | (SEQ | (SEQ | ID | ID | |
| | 259) | (SEQ | ID | ID | NO: | NO: | |
| | | ID | NO: | NO: | 302) | 303) | |
| | | NO: | 184) | 301) | | | |
| | | 182) | | | | | |
| CD2 | SNAVI | LIDVD | DYGSW | SGSNI | YNENR | GSYAG | 186-350 |
| | (SEQ | GDAAY | GYVSD | GDADV | PS | DTYNH | |
| | ID | DPALK | IDY | G | (SEQ | GV | |
| | NO: | S | (SEQ | (SEQ | ID | (SEQ | |
| | 198) | (SEQ | ID | ID | NO: | ID | |
| | | ID | NO: | NO: | 306) | NO: | |
| | | NO: | 202) | 305) | | 307) | |
| | | 200) | | | | | |
| CB9 | SNSVG | GIDSD | SYRAD | SGRFI | ASDGR | GSSDR | 239-348 |
| | (SEQ | GEEGY | GLAYG | GISSV | PS | TQYTG | |
| | ID | NPALN | YVQAI | G | (SEQ | V | |
| | NO: | S | DY | (SEQ | ID | (SEQ | |
| | 42) | (SEQ | (SEQ | ID | NO: | ID | |
| | | ID | ID | NO: | 70) | NO: | |
| | | NO: | NO: | 67) | | 74) | |
| | | 48) | 54) | | | | |
| CG11 | NYPVG | NIEND | EFGGS | SGSSS | GATSR | ASYDG | 266-359 |
| | (SEQ | GSANY | DGYTY | NVGYG | AS | SSSGV | |
| | ID | ASALK | FVDID | NYVS | (SEQ | (SEQ | |
| | NO: | S | Y | (SEQ | ID | ID | |
| | 310) | (SEQ | (SEQ | ID | NO: | NO: | |
| | | ID | ID | NO: | 141) | 314) | |
| | | NO: | NO: | 313) | | | |
| | | 311) | 312) | | | | |
| CA10 | SNGVG | DISSV | CRDGG | SGSSS | DATTR | AAHDS | 277-319 |
| | (SEQ | GKKYA | VTYGY | NVGYG | VS | SSGGV | |
| | ID | NPALK | DIDY | NYVT | (SEQ | (SEQ | |
| | NO: | S | (SEQ | (SEQ | ID | ID | |
| | 259) | (SEQ | ID | ID | NO: | NO: | |
| | | ID | NO: | NO: | 317) | 318) | |
| | | NO: | 161) | 316) | | | |
| | | 157) | | | | | |
| CC12 | SNSVG | GIDSD | SYRAD | SGRFI | ASDGR | GSSDR | 297-390 |
| | (SEQ | GEEGY | GLAYG | GISSV | PS | TQYTG | |
| | ID | NPALN | YVQAI | G | (SEQ | V | |
| | NO: | S | DY | (SEQ | ID | (SEQ | |
| | 42) | (SEQ | (SEQ | ID | NO: | ID | |
| | | ID | ID | NO: | 70) | NO: | |
| | | NO: | NO: | 67) | | 74) | |
| | | 48) | 54) | | | | |
| CE2/ | NYPVG | NIEND | EFGGS | SGSSS | GATSR | ASYDG | 319-331 |
| E1B8 | (SEQ | GSANY | DGYTY | NVGYG | AS | SSSGV | |
| | ID | ASALK | FVDID | NYVS | (SEQ | (SEQ | |
| | NO: | S | Y | (SEQ | ID | ID | |
| | 310) | (SEQ | (SEQ | ID | NO: | NO: | |
| | | ID | ID | NO: | 141) | 314) | |
| | | NO: | NO: | 313) | | | |
| | | 311) | 312) | | | | |
| CE3 | SNAVG | GCSSD | GYYPV | SGSSS | GTTSR | ASGDS | 331-360 |
| | (SEQ | GKCYY | YGYDY | NVGRN | PS | SAIND | |
| | ID | NSALK | LGTID | DVA | (SEQ | I | |
| | NO: | S | Y | (SEQ | ID | (SEQ | |
| | 17) | (SEQ | (SEQ | ID | NO: | ID | |
| | | ID | ID | NO: | 28) | NO: | |
| | | NO: | NO: | 25) | | 31) | |
| | | 20) | 23) | | | | |
| CA6 | SNGVG | DKSSG | CRDGG | SGSRN | DATTR | ASFDR | 348-390 |
| | (SEQ | GKTYG | VTYGY | NIGYG | AS | GSGGI | |
| | ID | NPALK | DIDY | NHVG | (SEQ | (SEQ | |
| | NO: | S | (SEQ | (SEQ | ID | ID | |
| | 259) | (SEQ | ID | ID | NO: | NO: | |
| | | ID | NO: | NO: | 207) | 325) | |
| | | NO: | 161) | 324) | | | |
| | | 323) | | | | | |
| CA11 | SNTVA | EINSG | GARST | SGSGS | GATSR | AAYDS | 348-441 |
| | (SEQ | GSTYY | YAAY | NIGAG | PS | GSSIV | |
| | ID | NPALK | (SEQ | NWVS | (SEQ | (SEQ | |
| | NO: | S | ID | (SEQ | ID | ID | |
| | 272) | (SEQ | NO: | ID | NO: | NO: | |
| | | ID | 274) | NO: | 328) | 329) | |
| | | NO: | | 327) | | | |
| | | 273) | | | | | |
| CA4 | SYSVY | IMYAS | GIEN | RTSQS | YATRL | LQYDS | 355-367 |
| | (SEQ | GRVDY | (SEQ | VNNYL | YT | TPLA | |
| | ID | NPALK | ID | S | (SEQ | (SEQ | |
| | NO: | S | NO: | (SEQ | ID | ID | |
| | 83) | (SEQ | 89) | ID | NO: | NO: | |
| | | ID | | NO: | 95) | 97) | |
| | | NO: | | 91) | | | |
| | | 84) | | | | | |
| CB2 | TNSVG | GIDTD | SYRTD | SGSYI | ASDGR | GSSDR | 359-391 |
| | (SEQ | GEEGF | GLAYG | GSSGV | PS | TQYTG | |
| | ID | NPVLK | YVQAI | G | (SEQ | L | |
| | NO: | S | DY | (SEQ | ID | (SEQ | |
| | 45) | (SEQ | (SEQ | ID | NO: | ID | |
| | | ID | ID | NO: | 70) | NO: | |
| | | NO: | NO: | 68) | | 79) | |
| | | 53) | 55) | | | | |
| CB3 | SVAVN | GIISN | GVEWE | SGSSS | GATKR | VSYQT | 360-390 |
| | (SEQ | GGTGY | GSMDY | NVGAG | AS | DFTLV | |
| | ID | NPALK | (SEQ | SYVG | (SEQ | (SEQ | |
| | NO: | S | ID | (SEQ | ID | ID | |
| | 334) | (SEQ | NO: | ID | NO: | NO: | |
| | | ID | 336) | NO: | 338) | 339) | |
| | | NO: | | 337) | | | |
| | | 335) | | | | | |
| CA9 | SNSVG | GIDTD | SYRSD | SGSFI | ASDGR | GSSDR | 367-379 |
| | (SEQ | GEEGY | GLAYG | GISSV | PS | TQYTG | |
| | ID | NPALN | YVQAI | G | (SEQ | V | |
| | NO: | S | DY | (SEQ | ID | (SEQ | |
| | 42) | (SEQ | (SEQ | ID | NO: | ID | |
| | | ID | ID | NO: | 70) | NO: | |
| | | NO: | NO: | 63) | | 74) | |
| | | 46) | 58) | | | | |
| CA12 | SYYVG | NIYST | GSYYH | SGSSS | AATSR | SSYQR | 367-379 |
| | (SEQ | GRAFY | GGGNG | NVGYG | AS | GNTGV | |
| | ID | NPALK | MVDFF | NYVG | (SEQ | (SEQ | |
| | NO: | S | DY | (SEQ | ID | ID | |
| | 44) | (SEQ | (SEQ | ID | NO: | NO: | |
| | | ID | ID | NO: | 72) | 78) | |
| | | NO: | NO: | 39) | | | |
| | | 52) | 265) | | | | |
| CB5 | SNGVG | DITSG | CRDGG | SGSSS | RTTNR | ASHDN | 49-113 |
| | (SEQ | GRTYG | VSYGY | NVGSG | AS | NSGGV | |
| | ID | NLALK | DSDY | DHVN | (SEQ | (SEQ | |
| | NO: | S | (SEQ | (SEQ | ID | ID | |
| | 259) | (SEQ | ID | ID | NO: | NO: | |
| | | ID | NO: | NO: | 343) | 344) | |
| | | NO: | 160) | 342) | | | |
| | | 341) | | | | | |
| CC4 | SNGVG | DIASS | CRDGG | SGSSS | GATNR | ASYDS | 49-155 |
| | (SEQ | GKAYS | VTYGY | NVGYT | AS | SNGGI | |
| | ID | NPALK | DIDY | NLGYS | (SEQ | (SEQ | |
| | NO: | S | (SEQ | NLVT | ID | ID | |
| | 259) | (SEQ | ID | (SEQ | NO: | NO: | |
| | | ID | NO: | ID | 170) | 347) | |
| | | NO: | 161) | NO: | | | |
| | | 158) | | 346) | | | |
| CD1 | SNGVG | DKSSA | CRDGG | SGSSS | GTTTR | ASYDT | 49-155 |
| | (SEQ | GKTYG | VSYGY | NVGYG | AS | GSGGV | |
| | ID | NPALK | DVDY | TYVS | (SEQ | (SEQ | |
| | NO: | S | (SEQ | (SEQ | ID | ID | |
| | 259) | (SEQ | ID | ID | NO: | NO: | |
| | | ID | NO: | NO: | 188) | 291) | |
| | | NO: | 286) | 290) | | | |
| | | 182) | | | | | |
| CC5 | SNGVG | DISSV | CRDGG | SGSSS | GATSR | ASYDS | 49-238 |
| | (SEQ | GKKYA | VTYGY | NVGYG | AS | SNGGV | |
| | ID | SPALK | DIDY | NYVG | (SEQ | (SEQ | |
| | NO: | S | (SEQ | (SEQ | ID | ID | |
| | 259) | (SEQ | ID | ID | NO: | NO: | |
| | | ID | NO: | NO: | 141) | 350) | |
| | | NO: | 161) | 39) | | | |
| | | 349) | | | | | |
| MD9/ | RESIA | GVGID | NYIDF | SGSSS | GTNNR | AAGDS | 373-385 |
| MoD9 | (SEQ | GTSYY | EY | NVGIY | PS | STIAV | |
| | ID | SPALK | (SEQ | DVS | (SEQ | (SEQ | |
| | NO: | S | ID | (SEQ | ID | ID | |
| | 102) | (SEQ | NO: | ID | NO: | NO: | |
| | | ID | 104) | NO: | 353) | 354) | |
| | | NO: | | 352) | | | |
| | | 103) | | | | | |
| NS1G7 | SYGVG | SISSG | DVHIY | SGSSS | GTTSR | ASYDT | 275-305, |
| | (SEQ | GTTFY | YNDYG | NIGGG | AS | NSGSV | 337-368 |
| | ID | NPALK | AAYGD | NYVS | (SEQ | (SEQ | |
| | NO: | S | RDY | (SEQ | ID | ID | |
| | 357) | (SEQ | (SEQ | ID | NO: | NO: | |
| | | ID | ID | NO: | 361) | 362) | |
| | | NO: | NO: | 360) | | | |
| | | 358) | 359) | | | | |
| 3bD11 | SYYVG | NIYST | GSYYH | SGSSS | AATSR | SSYQR | 37-49 |
| | (SEQ | GRAFY | GGGNG | NVGYG | AS | GNTGV | |
| | ID | NPALK | MVDFF | NYVG | (SEQ | (SEQ | |
| | NO: | S | DY | (SEQ | ID | ID | |
| | 44) | (SEQ | (SEQ | ID | NO: | NO: | |
| | | ID | ID | NO: | 72) | 78) | |
| | | NO: | NO: | 39) | | | |
| | | 52) | 265) | | | | |

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises a VHCDR1 amino acid sequence set forth in table 10;
VHCDR2 comprises a VHCDR2 amino acid sequence set forth in table 10;
VHCDR3 comprises a VHCDR3 amino acid sequence set forth in table 10;
VLCDR1 comprises a VLCDR1 amino acid sequence set forth in table 10;
VLCDR2 comprises a VLCDR2 amino acid sequence set forth in table 10; and
VLCDR3 comprises a VLCDR3 amino acid sequence set forth in table 10;
or for each CDR sequence, an amino acid sequence with
   (i) at least 85% identity thereto, and/or
   (ii) one, two, or three amino acid substitutions relative thereto,
wherein the specific binding molecule binds to an epitope within SEQ ID NO: 1, optionally wherein the specific binding molecule has a K_{D} of less than around 25 nM.

The K_{D} may be less than around 20 nM, less than around 15 nM, or less than around 10 nM. The K_{D} may preferably be for binding to SEQ ID NO: 1 or to SEQ ID NO: 5. The K_{D} for binding to SEQ ID NO: 1 may be around 1 nM to around 20 nM. The K_{D} for binding to SEQ ID NO: 1 may be around 1 nM to around 10 nM. The K_{D} for binding to SEQ ID NO: 1 may be around 1.23 nM to 6.9 nM, optionally wherein the specific binding molecule comprises the CDRs of CC7. The K_{D} for binding to SEQ ID NO: 1 may be around 1.3 nM to 3.61 nM, optionally wherein the specific binding molecule comprises the CDRs of CA4. The K_{D} for binding to SEQ ID NO: 1 may be around 3.79 nM to 16.7 nM, optionally wherein the specific binding molecule comprises the CDRs of CE3. The K_{D} for binding to SEQ ID NO: 1 may be around 5.03 nM to 11 nM, optionally wherein the specific binding molecule comprises the CDRs of CE2. The K_{D} for binding to SEQ ID NO: 1 may be around 3.7 nM to 5.19 nM, optionally wherein the specific binding molecule comprises the CDRs of CB7.

The specific binding molecule may comprise an amino acid sequence having at least 70%, at least 75%, at least 80%, at least 90%, at least 95% or at least 99% identity to SEQ ID NO: 363 wherein the specific binding molecule binds to a polypeptide or protein molecule comprising an amino acid sequence comprising residues 1-155 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 363. The CDRs may be 100% identical to the CDRs of SEQ ID NO: 363. The specific binding molecule may comprise an amino acid sequence having at least 70%, at least 75%, at least 80%, at least 90%, at least 95% or at least 99% identity to SEQ ID NO: 363 wherein CDRs are 100% identical to the CDRs of SEQ ID NO: 363. The specific binding molecule may comprise the amino acid sequence of SEQ ID NO: 363.

(CA2 amino acid sequence)
SEQ ID NO: 363
QVQLQESGPSLVKPSQTLSLTCTVSGFSLSSVAVNWVRQAPGKVP
EWLGGIISNGGTGYNPALKSRLSITRDTSKSQVSLALTHVTTEDT
AVYYCGRGVEWEGSMDYLGPGLLVTVSSEGKSSGSGSETKVDDQS
VLTQPSSVSGFLGQRVTITCSGSSSNVGAGSYVGWYQQVPGSGLR
ILIYGATKRASGLPDRFSGSRSGNTATLTISSLQAEDEADYYCVS
YQTDFTLVFGSGTRLTVLG

The specific binding molecule may comprise an amino acid sequence having at least 70%, at least 75%, at least 80%, at least 90%, at least 95% or at least 99% identity to SEQ ID NO: 364 wherein the specific binding molecule binds to a polypeptide or protein molecule comprising an amino acid sequence comprising residues 13-25 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 364. The CDRs may be 100% identical to the CDRs of SEQ ID NO: 364. The specific binding molecule may comprise an amino acid sequence having at least 70%, at least 75%, at least 80%, at least 90%, at least 95% or at least 99% identity to SEQ ID NO: 364 wherein CDRs are 100% identical to the CDRs of SEQ ID NO: 364. The specific binding molecule may comprise the amino acid sequence of SEQ ID NO: 364.

(CB7 amino acid sequence)
SEQ ID NO: 364
QVQLQESGPSLVKPSQTLSLTCTVSGFSLTNYRVGWVRQAPGKAL
EWVSNIRSGGTTWYNPALKSRLSITADTSKSQVSLSLSSVTTEDT
AVYYCARDSSGDLYAYDYWGPGLLVTVSSEGKSSGASGESKVDDQ
AVLTQPSSVSRSLGQSVSMTCSGSSSNVGYGNYMAWFQQVPGSAP
KLLIYGATSRASGVPDRFSGSRSGNTATLTISSLQAEDEADYYCA
SYDSTSGGVFGSGTRLTVLG

The specific binding molecule may comprise an amino acid sequence having at least 70%, at least 75%, at least 80%, at least 90%, at least 95% or at least 99% identity to SEQ ID NO: 365 wherein the specific binding molecule binds to a polypeptide or protein molecule comprising an amino acid sequence comprising residues 145 to 157 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 365. The CDRs may be 100% identical to the CDRs of SEQ ID NO: 365. The specific binding molecule may comprise an amino acid sequence having at least 70%, at least 75%, at least 80%, at least 90%, at least 95% or at least 99% identity to SEQ ID NO: 365 wherein CDRs are 100% identical to the CDRs of SEQ ID NO: 365. The specific binding molecule may comprise the amino acid sequence of SEQ ID NO: 365.

(CC7 amino acid sequence)
SEQ ID NO: 365
QVQLQESGPSLVKPSQTLSLTCTVSGFSLTSNAVIWVRQAPGKAP
EWVALIDVDGDAAYDPALKSRLSITRDTSKSQVSLSLRSVTTEDT
AVYYCARDYGSWGYVSDIDYWGPGLLVTVSSEGKSSGASGESKVD
DRVVRTQPSSVSGSLGQRVSITCSGSYITGSSVGWFQQVPGSGLK
TVIYDNNDRPSGVPDRFSGSKSGDTATLTISSLQAEDEADYYCAS
YDTSNIGLFGSGTRLTVLG

The specific binding molecule may comprise an amino acid sequence having at least 70%, at least 75%, at least 80%, at least 90%, at least 95% or at least 99% identity to SEQ ID NO: 366 wherein the specific binding molecule binds to a polypeptide or protein molecule comprising an amino acid sequence comprising residues 319 to 331 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 366. The CDRs may be 100% identical to the CDRs of SEQ ID NO: 366. The specific binding molecule may comprise an amino acid sequence having at least 70%, at least 75%, at least 80%, at least 90%, at least 95% or at least 99% identity to SEQ ID NO: 366 wherein CDRs are 100% identical to the CDRs of SEQ ID NO: 366. The specific binding molecule may comprise the amino acid sequence of SEQ ID NO: 366.

(CE2/E1B8 amino acid sequence)
SEQ ID NO: 366
QVQLQESGPSLVKPSQTLSLTCTVSGFSLTNYPVGWVRQAPGKAL
EWIGNIENDGSANYASALKSRLSITRDTSKNQVSLSLSSATTEDT
AVYYCGREFGGSDGYTYFVDIDYWGPGLLVTVSSEGKSSGASGES
KVDDQAVLTQPSSVSKSLGQSVSITCSGSSSNVGYGNYVSWFQQV
PGSAPKILIYGATSRASGVPDRFSGSRSGNTATLTITSLQAEDEA
DYYCASYDGSSSGVFGSGTRLTVLG

The specific binding molecule may comprise an amino acid sequence having at least 70%, at least 75%, at least 80%, at least 90%, at least 95% or at least 99% identity to SEQ ID NO: 367 wherein the specific binding molecule binds to a polypeptide or protein molecule comprising an amino acid sequence comprising residues 331 to 360 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 367. The CDRs may be 100% identical to the CDRs of SEQ ID NO: 367. The specific binding molecule may comprise an amino acid sequence having at least 70%, at least 75%, at least 80%, at least 90%, at least 95% or at least 99% identity to SEQ ID NO: 367 wherein CDRs are 100% identical to the CDRs of SEQ ID NO: 367. The specific binding molecule may comprise the amino acid sequence of SEQ ID NO: 367.

SEQ ID NO 367:
(CE3 amino acid sequence)
QVRLQESGPSLVKPSQTLSVTCTVSGFSLISNAVGWVRQAPGKVP
ESLAGCSSDGKCYYNSALKSRLDITRDTSKNQISLSLSSVTTDDA
AVYYCTRGYYPVYGYDYLGTIDYWGPGLLVTVSSEGKSSGASGES
KVDDQAVLTQPSSVSGSLGQRVSITCSGSSSNVGRNDVAWFQQLP
GSGLRTIIYGTTSRPSGIPDRFSGSKSGVTATLTIDSLQAEDEAD
YFCASGDSSAINDIFGSGTRLTVLG

The specific binding molecule may comprise an amino acid sequence having at least 70%, at least 75%, at least 80%, at least 90%, at least 95% or at least 99% identity to SEQ ID NO: 368 wherein the specific binding molecule binds to a polypeptide or protein molecule comprising an amino acid sequence comprising residues 355 to 367 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 368. The CDRs may be 100% identical to the CDRs of SEQ ID NO: 368. The specific binding molecule may comprise an amino acid sequence having at least 70%, at least 75%, at least 80%, at least 90%, at least 95% or at least 99% identity to SEQ ID NO: 368 wherein CDRs are 100% identical to the CDRs of SEQ ID NO: 368. The specific binding molecule may comprise the amino acid sequence of SEQ ID NO: 368.

(CA4 amino acid sequence)
SEQ ID NO: 368
QVQLQESGPSLVKPSQTLSLTCTVSGFSLTSYSVYWVRQAPGQALEWIS
IMYASGRVDYNPALKSRLSITRDTSKSQFSLSLSSVTTEDTAVYYCTRG
IENWGPGLLVTVSSEGKSSGASGESKVDDDIQVTQSPSSLSASLTERVS
ITCRTSQSVNNYLSWYQQKPGQAPKLLIYYATRLYTDVPSRFSGSGSGT
DYTLTITSLEADDTATYYCLQYDSTPLAFGGGTNVEIK

The specific binding molecule may comprise an amino acid sequence having at least 70%, at least 75%, at least 80%, at least 90%, at least 95% or at least 99% identity to SEQ ID NO: 369 wherein the specific binding molecule binds to a polypeptide or protein molecule comprising an amino acid sequence comprising residues 359 to 391 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 369. The CDRs may be 100% identical to the CDRs of SEQ ID NO: 369. The specific binding molecule may comprise an amino acid sequence having at least 70%, at least 75%, at least 80%, at least 90%, at least 95% or at least 99% identity to SEQ ID NO: 369 wherein CDRs are 100% identical to the CDRs of SEQ ID NO: 369. The specific binding molecule may comprise the amino acid sequence of SEQ ID NO: 369.

(CB2 amino acid sequence)
SEQ ID NO: 369
QVQLQESGPSLVKPSQTLSLTCTVSGFSLSTNSVGWVRQAPGKAPEWVA
GIDTDGEEGFNPVLKSRLSITRDTSKSQVSLSLSNVTSEDTAVYYCGRS
YRTDGLAYGYVQAIDYWGPGLLVTISSEGKSSGASGESKVDDQSVLTQP
SSVSGSPGQTVSITCSGSYIGSSGVGWFQQLPGSGLRTIIVASDGRPSG
VPDRFSMSKSGNTATLTISSLQAEDEADYFCGSSDRTQYTGLFGSGTRL
TVLG

The specific binding molecule may comprise an amino acid sequence having at least 70%, at least 75%, at least 80%, at least 90%, at least 95% or at least 99% identity to SEQ ID NO: 370 wherein the specific binding molecule binds to a polypeptide or protein molecule comprising an amino acid sequence comprising residues 360 to 390 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 370. The CDRs may be 100% identical to the CDRs of SEQ ID NO: 370. The specific binding molecule may comprise an amino acid sequence having at least 70%, at least 75%, at least 80%, at least 90%, at least 95% or at least 99% identity to SEQ ID NO: 370 wherein CDRs are 100% identical to the CDRs of SEQ ID NO: 370. The specific binding molecule may comprise the amino acid sequence of SEQ ID NO: 370.

(CB3 amino acid sequence)
SEQ ID NO: 370
QVQLQESGPSLVKPSQTLSLTCTVSGFSLSSVAVNWVRQAPGKVPEWLG
GIISNGGTGYNPALKSRLSITRDTSKSQVSLALTHVTTEDTAVYYCGRG
VEWEGSMDYLGPGLLVTVSSEGKSSGSGSETKVDDQSVLTQPSSVSGFL
GQRVTITCSGSSSNVGAGSYVGWYQQVPGSGLRILIYGATKRASGLPDRF
SGSRSGNTATLTISSLQAEDEADYYCVSYQTDFTLVFGSGTRLTVLG

The specific binding molecule may comprise an amino acid sequence having at least 70%, at least 75%, at least 80%, at least 90%, at least 95% or at least 99% identity to SEQ ID NO: 371 wherein the specific binding molecule binds to a polypeptide or protein molecule comprising an amino acid sequence comprising residues 367 to 379 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 371. The CDRs may be 100% identical to the CDRs of SEQ ID NO: 371. The specific binding molecule may comprise an amino acid sequence having at least 70%, at least 75%, at least 80%, at least 90%, at least 95% or at least 99% identity to SEQ ID NO: 371 wherein CDRs are 100% identical to the CDRs of SEQ ID NO: 371. The specific binding molecule may comprise the amino acid sequence of SEQ ID NO: 371.

(CA9 amino acid sequence)
SEQ ID NO: 371
QVQLQESGPSLVKPSQTLSLTCTVSGFSLTSNSVGWVRQAPGKAPEWVA
GIDTDGEEGYNPALNSRLSITRDTSKSQVSLSLSSVTSEDTAVYYCGRS
YRSDGLAYGYVQAIDYWGPGLLVTVSSEGKSSGASGESKVDDQAVLTQP
ASVSGSLGQRVSITCSGSFIGISSVGWFQQLPGSGLRTIIVASDGRPSG
VPDRFSMSKSGNTATLTISSLQAEDEADYFCGSSDRTQYTGVFGSGTRL
TVLG

The specific binding molecule may comprise an amino acid sequence having at least 70%, at least 75%, at least 80%, at least 90%, at least 95% or at least 99% identity to SEQ ID NO: 372 wherein the specific binding molecule binds to a polypeptide or protein molecule comprising an amino acid sequence comprising residues 367 to 379 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 372. The CDRs may be 100% identical to the CDRs of SEQ ID NO: 372. The specific binding molecule may comprise an amino acid sequence having at least 70%, at least 75%, at least 80%, at least 90%, at least 95% or at least 99% identity to SEQ ID NO: 372 wherein CDRs are 100% identical to the CDRs of SEQ ID NO: 372. The specific binding molecule may comprise the amino acid sequence of SEQ ID NO: 372.

(CA12 amino acid sequence)
SEQ ID NO: 372
QVQLQGSGPSLVKPSQTLSLTCTVSGFSFDSYYVGWVRQAPGKALEWLG
NIYSTGRAFYNPALKSRLSITRDTSKSQVSLSVSSVTIEDTALYYCVRG
SYYHGGGNGMVDFFDYWSPGLLVTVSSEGKSSGASGESKVDDQVVRTQP
SSVSGSLGQRVSITCSGSSSNVGYGNYVGWFQQVPGSAPKLLIYAATSR
ASGVPDRFSGSRSGNTATLTIDSLQAEDEADYYCSSYQRGNTGVFGSGT
RLTVLG

The specific binding molecule may comprise an amino acid sequence having at least 70%, at least 75%, at least 80%, at least 90%, at least 95% or at least 99% identity to SEQ ID NO: 373 wherein the specific binding molecule binds to a polypeptide or protein molecule comprising an amino acid sequence comprising residues 373 to 385 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 373. The CDRs may be 100% identical to the CDRs of SEQ ID NO: 373. The specific binding molecule may comprise an amino acid sequence having at least 70%, at least 75%, at least 80%, at least 90%, at least 95% or at least 99% identity to SEQ ID NO: 373 wherein CDRs are 100% identical to the CDRs of SEQ ID NO: 373. The specific binding molecule may comprise the amino acid sequence of SEQ ID NO: 373.

(MD9/MoD9 amino acid sequence)
SEQ ID NO: 373
QVRLQESGPSLVKSSQTLSLTCTVSGFSLTRESIAWVRQAPGKVPEWLG
GVGIDGTSYYSPALKSRLSITRDTSKSQASLSLSSVATEDTAMYYCARN
YIDFEYWGPGLLVTVSSEGKSSGASGESKVDDQAVLTQLSSVSGSLGQR
ISITCSGSSSNVGIYDVSWFQQLPGSGLRTVIYGTNNRPSGVPDRFSGS
RSGNTATLTISSLQSEDEAIYYCAAGDSSTIAVFGSGTRLTVLG

The specific binding molecule may comprise an amino acid sequence having at least 70%, at least 75%, at least 80%, at least 90%, at least 95% or at least 99% identity to SEQ ID NO: 637 wherein the specific binding molecule binds to a polypeptide or protein molecule comprising an amino acid sequence comprising residues 37 to 49 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 637. The CDRs may be 100% identical to the CDRs of SEQ ID NO: 637. The specific binding molecule may comprise an amino acid sequence having at least 70%, at least 75%, at least 80%, at least 90%, at least 95% or at least 99% identity to SEQ ID NO: 637 wherein CDRs are 100% identical to the CDRs of SEQ ID NO: 637. The specific binding molecule may comprise the amino acid sequence of SEQ ID NO: 637.

(3bD11 amino acid sequence)
SEQ ID NO: 637
RVRLQGSGPSLVKPSQTLSLTCTVSGFSFDSYYVGWVRQAPGKALEWLG
NIYSTGRAFYNPALKSRLSITRDTSKSQVSLSVSSVTIEDTALYYCVRG
SYYHGGGNGMVDFFDYWSPGLLVTVSSEGKSSGASGESKVDDQVVRTQP
SSVSGSLGQRVSITCSGSSSNVGYGNYVGWFQQVPGSAPKLLIYAATSR
ASGVPDRFSGSRSGNTATLTIDSLQAEDEADYYCSSYQRGNTGVFGSGT
RLTVLG

The specific binding molecule may comprise an amino acid sequence having at least 70%, at least 75%, at least 80%, at least 90%, at least 95% or at least 99% identity to SEQ ID NO: 374 wherein the specific binding molecule binds to a polypeptide or protein molecule comprising an amino acid sequence comprising residues 275 to 305 and/or 337 to 368 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 374. The CDRs may be 100% identical to the CDRs of SEQ ID NO: 374. The specific binding molecule may comprise an amino acid sequence having at least 70%, at least 75%, at least 80%, at least 90%, at least 95% or at least 99% identity to SEQ ID NO: 374 wherein CDRs are 100% identical to the CDRs of SEQ ID NO: 374. The specific binding molecule may comprise the amino acid sequence of SEQ ID NO: 374.

(NS1G7 amino acid sequence)
SEQ ID NO: 374
QVQLQESGPSLVKPSQTLSLTCTVSGFSLTSYGVGWVRQAPGKTLEWIS
SISSGGTTFYNPALKSRLSITRDTSESQVSLSLSSVTTEDTAVYYCTRD
VHIYYNDYGAAYGDRDYWGPGLLVTVSSEGKSSGASGESKVDDQAVVTQ
PPSVSGSPGQRVSITCSGSSSNIGGGNYVSWYQQLPGSGLRTLIYGTTS
RASGVPDRFSGSGSGNTATLTISSLQAEDEADYYCASYDTNSGSVFGS
GTRLTVLG

The epitope of the specific binding molecule may be within an amino acid sequence comprising residues 297 to 390 of SEQ ID NO: 1. Accordingly, the epitope may be within the amino acid sequence of SEQ ID NO: 5 (IKHVPGGGSVQIVYKPVDLSKVTSKCGSLGNIHHKPGGGQVEVKSEKLDFKDRVQSKIGSLDNITH VPGGGNKKIETHKLTFRENAKAKTDHGA).

The epitope may be within an amino acid sequence comprising residues 297 to 390 of SEQ ID NO: 1. This epitope may be bound by the CDRs of the specific binding molecule referred to as “S1E12” herein.

The epitope may comprise the amino acid sequence of SEQ ID NO: 5 (IKHVPGGGSVQIVYKPVDLSKVTSKCGSLGNIHHKPGGGQVEVKSEKLDFKDRVQSKIGSLDNITH VPGGGNKKIETHKLTFRENAKAKTDHGA). The epitope may comprise an amino acid sequence having at least 70%, at least 75%, at least 80%, at least 90%, at least 95% or at least 99% identity to SEQ ID NO 5 (IKHVPGGGSVQIVYKPVDLSKVTSKCGSLGNIHHKPGGGQVEVKSEKLDFKDRVQSKIGSLDNITH VPGGGNKKIETHKLTFRENAKAKTDHGA).

The epitope may consist of the amino acid sequence of SEQ ID NO: 5 (IKHVPGGGSVQIVYKPVDLSKVTSKCGSLGNIHHKPGGGQVEVKSEKLDFKDRVQSKIGSLDNITH VPGGGNKKIETHKLTFRENAKAKTDHGA). The epitope may consist of an amino acid sequence having at least 70%, at least 75%, at least 80%, at least 90%, at least 95% or at least 99% identity to SEQ ID NO: 5 (IKHVPGGGSVQIVYKPVDLSKVTSKCGSLGNIHHKPGGGQVEVKSEKLDFKDRVQSKIGSLDNITH VPGGGNKKIETHKLTFRENAKAKTDHGA).

The specific binding molecule may bind to a polypeptide or protein molecule comprising an amino acid sequence comprising residues 297 to 390 of SEQ ID NO: 1. Said specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 136 (S/R/D/T N/E/Y/H S/G V/I G/A);
VHCDR2 comprises the sequence set forth in SEQ ID NO: 162 (G I/V D/G/N T/I/Y/S D G E/T/R E/S/T G/Y/E Y/F N/S P/S A/V L N/K S);
VHCDR3 comprises the sequence set forth in SEQ ID NO: 164 (S/N/D/T Y/-/S R/-/K A/-/G/S/T D/-G/-L/-/Y A/-/G Y/-/W G/-Y/-/H V/Y Q/I/Y A/D/Q I/F D/E Y);
VLCDR1 comprises the sequence set forth in SEQ ID NO: 167 (S G S F/N/S/Y I/S G/N I/S/V S/A/G S/Y/G-/G-/D-/Y V G/T/S);
VLCDR2 comprises the sequence set forth in SEQ ID NO: 181 (A/R/D S/N/A D/R/T G/N R P/A S); and
VLCDR3 comprises the sequence set forth in SEQ ID NO: 183 (G/A S S/Y/H D/-R/Q/D T/S/N Q/W/R Y/G/I T/S G/A V/L);
or for each CDR sequence, an amino acid sequence with
   (i) at least 85% identity thereto, and/or
   (ii) one, two, or three amino acid substitutions relative thereto.

Said sequence identity is at least about 85% sequence identity and may therefore be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity. Preferably said sequence identity is at least 90% or at least 95%.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG), SEQ ID NO: 187 (SHSVG), SEQ ID NO: 45 (TNSVG), SEQ ID NO: 102 (RESIA), or SEQ ID NO: 199 (DYGIG);
VHCDR2 comprises the sequence set forth in SEQ ID NO: 46 (GIDTDGEEGYNPALNS), SEQ ID NO: 205 (GINYDGRTEYNSALKS), SEQ ID NO: 103 (GVGIDGTSYYSPALKS), SEQ ID NO: 53 (GIDTDGEEGFNPVLKS), or SED ID NO: 48 (GIDSDGEEGYNPALNS);
VHCDR3 comprises the sequence set forth in SEQ ID NO: 54 (SYRADGLAYGYVQAIDY), SEQ ID NO: 55 (SYRTDGLAYGYVQAIDY), SEQ ID NO: 210 (TYRSDGYAYGYVQAIDY), SEQ ID NO: 104 (NYIDFEY), or SEQ ID NO: 211 (DSKGGWGHVYQFDY);
VLCDR1 comprises the sequence set forth in SEQ ID NO: 63 (SGSFIGISSVG), SEQ ID NO: 68 (SGSYIGSSGVG), SEQ ID NO: 212 (SGSNIGSASVT), or SEQ ID NO: 213 (SGSSSNVGYGDYVS);
VLCDR2 comprises the sequence set forth in SEQ ID NO: 70 (ASDGRPS), SEQ ID NO: 214 (RNRNRPS), or SEQ ID NO: 215 (DATNRAS); and
VLCDR3 comprises the sequence set forth in SEQ ID NO: 74 (GSSDRTQYTGV), SEQ ID NO: 79 (GSSDRTQYTGL), SEQ ID NO: 216 (ASHDNRISAV), or SEQ ID NO: 217 (GSYQSWGSGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 297 to 390 of SEQ ID NO: 1.

The specific binding molecule may bind to a polypeptide or protein molecule comprising an amino acid sequence comprising residues 297 to 390 of SEQ ID NO: 1. Said specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 426 (S/T N/H S V G);
VHCDR2 comprises the sequence set forth in SEQ ID NO: 427 (G I D T/S D G E E G Y/F N P A/V L N/K S);
VHCDR3 comprises the sequence set forth in SEQ ID NO: 428 (S/T Y R A/T/S D G L Y G Y V Q A I D Y);
VLCDR1 comprises the sequence set forth in SEQ ID NO: 429 (S G S F/Y I G I/S S S/G V G);
VLCDR2 comprises the sequence set forth in SEQ ID NO: 70 (A S D G R P S); and
VLCDR3 comprises the sequence set forth in SEQ ID NO: 432 (G S S D R T Q Y T G V/L); or for each CDR sequence, an amino acid sequence with
   (i) at least 85% identity thereto, and/or
   (ii) one, two, or three amino acid substitutions relative thereto.

Said sequence identity is at least about 85% sequence identity and may therefore be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity. Preferably said sequence identity is at least 90% or at least 95%.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG), SEQ ID NO: 187 (SHSVG) or SEQ ID NO: 45 (TNSVG);
VHCDR2 comprises the sequence set forth in SEQ ID NO: 46 (GIDTDGEEGYNPALNS), SEQ ID NO: 53 (GIDTDGEEGFNPVLKS), or SED ID NO: 48 (GIDSDGEEGYNPALNS);
VHCDR3 comprises the sequence set forth in SEQ ID NO: 54 (SYRADGLAYGYVQAIDY), SEQ ID NO: 55 (SYRTDGLAYGYVQAIDY), or SEQ ID NO: 210 (TYRSDGYAYGYVQAIDY);
VLCDR1 comprises the sequence set forth in SEQ ID NO: 63 (SGSFIGISSVG), or SEQ ID NO: 68 (SGSYIGSSGVG);
VLCDR2 comprises the sequence set forth in SEQ ID NO: 70 (ASDGRPS); and
VLCDR3 comprises the sequence set forth in SEQ ID NO: 74 (GSSDRTQYTGV), or SEQ ID NO: 79 (GSSDRTQYTGL);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 297 to 390 of SEQ ID NO: 1.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 54 (SYRADGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS); and
VLCDR3 comprises the sequence set forth in SEQ ID NO: 74 (GSSDRTQYTGV);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 297 to 390 of SEQ ID NO: 1. The specific binding molecule comprising CDRs having 100% identity to those given above is referred to as “S1E12” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 54 (SYRADGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS); and
VLCDR3 comprises the sequence set forth in SEQ ID NO: 74 (GSSDRTQYTGV).

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 45 (TNSVG);
VHCDR2 comprises the sequence set forth in SEQ ID NO: 53 (GIDTDGEEGFNPVLKS);
VHCDR3 comprises the sequence set forth in SEQ ID NO: 55 (SYRTDGLAYGYVQAIDY);
VLCDR1 comprises the sequence set forth in SEQ ID NO: 68 (SGSYIGSSGVG);
VLCDR2 comprises the sequence set forth in SEQ ID NO: 70 (ASDGRPS); and
VLCDR3 comprises the sequence set forth in SEQ ID NO: 79 (GSSDRTQYTGL);
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 297 to 390 of SEQ ID NO: 1. The specific binding molecule comprising CDRs having 100% identity to those given above is referred to as “NS2A1” herein.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 45 (TNSVG);
VHCDR2 comprises the sequence set forth in SEQ ID NO: 53 (GIDTDGEEGFNPVLKS);
VHCDR3 comprises the sequence set forth in SEQ ID NO: 55 (SYRTDGLAYGYVQAIDY);
VLCDR1 comprises the sequence set forth in SEQ ID NO: 68 (SGSYIGSSGVG);
VLCDR2 comprises the sequence set forth in SEQ ID NO: 70 (ASDGRPS); and
VLCDR3 comprises the sequence set forth in SEQ ID NO: 79 (GSSDRTQYTGL).

The specific binding molecule may comprise the CDR sequences of a clone set out in Table 11 below. The epitope may be within residues 297 to 390 of SEQ ID NO: 1.

TABLE 11

| Clone | VH | | | VL | | | |
|---|---|---|---|---|---|---|---|
| name | CDR1 | CDR2 | CDR3 | CDR1 | CDR2 | CDR3 | Epitope |
| NS2B6 | RESIA | GVGIDGTSYYSPALK | N----------YID | SGSNIGSAS--- | RNRNRPS | GSY- | 297-390 |
| | (SEQ ID | S | FEY | VT | (SEQ ID | QSWGSGV | |
| | NO: 102) | (SEQ ID NO: 103) | (SEQ ID NO: 104) | (SEQ ID | NO: 214) | (SEQ ID | |
| | | | | NO: 212) | | NO: 217) | |
| NS1B2 | DYGIG | GINYDGRTEYNSALK | DSKG--- | SGSSSNVGYGDY | DATNRAS | ASH-DNRI | 297-390 |
| | (SEQ ID | S | GWGHVYQFDY (SEQ | SV | (SEQ ID | SAV | |
| | NO: 199) | (SEQ ID NO: 205) | ID NO: 211) | (SEQ ID | NO: 215) | (SEQ ID | |
| | | | | NO: 213) | | NO: 216) | |
| S1A5 | SNSVG | GIDTDGEEGYNPALN | TYRSDGYAYGYVQAID | NA | NA | NA | 297-390 |
| | (SEQ ID | S | Y | | | | |
| | NO: 42) | (SEQ ID NO: 46) | (SEQ ID NO: 210) | | | | |
| S1A12 | SNSVG | GIDTDGEEGYNPALN | TYRSDGYAYGYVQAID | SGSFIGISS---VG | ASDGRPS | GSSDRTQYTG | 297-390 |
| | (SEQ ID | S | Y | (SEQ ID NO: 63) | (SEQ ID | V (SEQ | |
| | NO: 42) | (SEQ ID NO: 46) | (SEQ ID NO: 210) | | NO: 70) | ID NO: 74) | |
| S1E12 | SNSVG | GIDTDGEEGYNPALN | SYRADGLAYGYVQAID | SGSFIGISS---VG | ASDGRPS | GSSDRTQYTG | 297-390 |
| | (SEQ ID | S | Y | (SEQ ID NO: 63) | (SEQ ID | V (SEQ ID | |
| | NO: 42) | (SEQ ID NO: 46) | (SEQ ID NO: 54) | | NO: 70) | NO: 74) | |
| S1D5 | SHSVG | GIDSDGEEGYNPALN | SYRADGLAYGYVQAID | SGSFIGISS---VG | ASDGRPS | GSSDRTQYTG | 297-390 |
| | (SEQ ID | S | Y | (SEQ ID NO: 63) | (SEQ ID | V (SEQ ID | |
| | NO: 187) | (SEQ ID NO: 48) | (SEQ ID NO: 54) | | NO: 70) | NO: 74) | |
| NS2A1 | TNSVG | GIDTDGEEGFNPVLK | SYRTDGLAYGYVQAID | SGSYIGSSG---VG | ASDGRPS | GSSDRTQYTG | 297-390 |
| | (SEQ ID | S | Y | (SEQ ID NO: 68) | (SEQ ID | L (SEQ ID | |
| | NO: 45) | (SEQ ID NO: 53) | (SEQ ID NO: 55) | | NO: 70) | NO: 79) | |

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises a VHCDR1 amino acid sequence set forth in table 11;
VHCDR2 comprises a VHCDR2 amino acid sequence set forth in table 11;
VHCDR3 comprises a VHCDR3 amino acid sequence set forth in table 11;
VLCDR1 comprises a VLCDR1 amino acid sequence set forth in table 11;
VLCDR2 comprises a VLCDR2 amino acid sequence set forth in table 11; and
VLCDR3 comprises a VLCDR3 amino acid sequence set forth in table 11;
or for each CDR sequence, an amino acid sequence with
   (i) at least 85% identity thereto, and/or
   (ii) one, two, or three amino acid substitutions relative thereto,
wherein the specific binding molecule binds to a polypeptide or protein molecule comprising an amino acid sequence comprising residues 297 to 390 of SEQ ID NO: 1.

The specific binding molecule may bind to a polypeptide or protein molecule comprising an amino acid sequence comprising residues 297 to 390 of SEQ ID NO: 1 with a K_{D} of less than around 25 nM. The K_{D} may be less than around 20 nM, less than around 15 nM, or less than around 10 nM. The K_{D} may preferably be for binding to SEQ ID NO: 1 or SEQ ID NO: 5. The K_{D} for binding to SEQ ID NO: 1 may be around 500 pM to around 15 nM. The K_{D} for binding to SEQ ID NO: 1 may be around 500 pM to around 1 nM. The K_{D} for binding to SEQ ID NO: 1 may be around 829 pM, optionally wherein the specific binding molecule comprises the CDRs of S1E12. The K_{D} for binding to SEQ ID NO: 1 may be around 1 nM to around 15 nM. The K_{D} for binding to SEQ ID NO: 1 may be around 2.9 nM to 10 nM, optionally wherein the specific binding molecule comprises the CDRs of NS2A1. The K_{D} for binding to SEQ ID NO: 5 may be around 1 nM to around 15 nM. The K_{D} for binding to SEQ ID NO: 1 may be around 3 nM to 8 nM. The K_{D} for binding to SEQ ID NO: 1 may be around 5.4 nM, optionally wherein the specific binding molecule comprises the CDRs of NS2A1.

The specific binding molecule may comprise an amino acid sequence having at least 70%, at least 75%, at least 80%, at least 90%, at least 95% or at least 99% identity to SEQ ID NO: 218 wherein the specific binding molecule binds to a polypeptide or protein molecule comprising an amino acid sequence comprising residues 297 to 390 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 218. The CDRs may be 100% identical to the CDRs of SEQ ID NO: 218. The specific binding molecule may comprise an amino acid sequence having at least 70%, at least 75%, at least 80%, at least 90%, at least 95% or at least 99% identity to SEQ ID NO: 218 wherein CDRs are 100% identical to the CDRs of SEQ ID NO: 218. The specific binding molecule may comprise the amino acid sequence of SEQ ID NO: 218.

(S1E12 amino acid sequence)
SEQ ID NO: 218
QVQLQESGPSLVKPSQTLSLTCTVSGFSLGSNSVGWVRQAPGKAPEWVA
GIDTDGEEGYNPALNSRLSITRDTSKSQVSLSLSSVTSEDTAVYYCGRS
YRADGLAYGYVQAIDYWGPGLLVTVSSEGKSSGASGESKVDDRVVRTQP
SSVSGSLGQRVSITCSGSFIGISSVGWFQQLPGSGLRTIIVASDGRPSG
VPDRFSMSKSGNTATLTISSLQAEDEADYFCGSSDRTQYTGVFGSGTRL
TVLG

The specific binding molecule may comprise an amino acid sequence having at least 70%, at least 75%, at least 80%, at least 90%, at least 95% or at least 99% identity to SEQ ID NO: 220 wherein the specific binding molecule binds to a polypeptide or protein molecule comprising an amino acid sequence comprising residues 297 to 390 of SEQ ID NO: 1. The CDRs of the specific binding molecule may be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the CDRs of SEQ ID NO: 220. The CDRs may be 100% identical to the CDRs of SEQ ID NO: 220. The specific binding molecule may comprise an amino acid sequence having at least 70%, at least 75%, at least 80%, at least 90%, at least 95% or at least 99% identity to SEQ ID NO: 220 wherein CDRs are 100% identical to the CDRs of SEQ ID NO: 220. The specific binding molecule may comprise the amino acid sequence of SEQ ID NO: 220.

(NS2A1 amino acid sequence)
SEQ ID NO: 220
QVQLQESGPSLVKPSQTLSLTCTVSGFSLSTNSVGWVRQAPGKAPEWVA
GIDTDGEEGFNPVLKSRLSITRDTSKSQVSLSLSNVTSEDTAVYYCGRS
YRTDGLAYGYVQAIDYWGPGLLVTISSEGKSSGASGESKVDDQSVLTQP
SSVSGSPGQTVSITCSGSYIGSSGVGWFQQLPGSGLRTIIVASDGRPSG
VPDGFSMSKSGNTATLTISSLQAEDEADYFCGSSDRTQYTGLFGSGTRL
TVLG

CDRs disclosed herein in connection with different clones may be combined into a specific binding molecule.

The specific binding molecule may comprise one or more CDR sequences disclosed herein in connection a clone identified in any one of tables 1 to 11 wherein the remaining CDR sequences are disclosed herein in connection one or more other clones identified in any one of tables 1 to 11. The specific binding molecule may comprise CDRs from two, three, four, five or six clones identified in any one of tables 1 to 11.

The specific binding molecule may comprise the CDRs VHCDR1, VHCDR2, VHCDR3, VLCDR1, VLCDR2 and VLCDR3, wherein each of said CDRs comprises an amino acid sequence as follows:
VHCDR1 comprises a VHCDR1 amino acid sequence set forth in any one of tables 1 to 11;
VHCDR2 comprises a VHCDR2 amino acid sequence set forth in any one of tables 1 to 11;
VHCDR3 comprises a VHCDR3 amino acid sequence set forth in any one of tables 1 to 11;
VLCDR1 comprises a VLCDR1 amino acid sequence set forth in any one of tables 1 to 11;
VLCDR2 comprises a VLCDR2 amino acid sequence set forth in any one of tables 1 to 11; and
VLCDR3 comprises a VLCDR3 amino acid sequence set forth in any one of tables 1 to 11;
or for each CDR sequence, an amino acid sequence with
   (i) at least 85% identity thereto, and/or
   (ii) one, two, or three amino acid substitutions relative thereto,
wherein the specific binding molecule binds to an epitope within SEQ ID NO: 1.

The specific binding molecule may comprise the CDRs of a clone selected from the group consisting of S1D12, E2E8, E1E8, E2A6, E2B7, NS2A1, S1E12, S1B1, S1D9, S1F4, S1G2, S1G10, S2C6, MD9, 412E10, 412B9, 412E6, 412G11, CA2, CA4, CA9, CA12, CB2, CB3, CB7, CC7, CE2, CE3, 3aA6, 3aD6, 3aB7, 3bF4, 3aD3, 3aH6, 3aG3, 3bG4 and NS1G7;
or for each CDR sequence, an amino acid sequence with
   (i) at least 85% identity thereto, and/or
   (ii) one, two, or three amino acid substitutions relative thereto,
   wherein the specific binding molecule binds to an epitope within SEQ ID NO: 1.

The specific binding molecule may comprise the CDRs of a clone selected from the group consisting of S1D12, E2E8, E1E8, E2A6, E2B7, NS2A1, S1E12, S1B1, S1D9, S1F4, S1G2, S1G10, S2C6, MD9, 412E10, 412B9, 412E6, 412G11, CA2, CA4, CA9, CA12, CB2, CB3, CB7, CC7, CE2, CE3, 3aA6, 3aD6, 3aB7, 3bF4, 3aD3, 3aH6, 3aG3, 3bG4 and NS1G7.

Without being bound by theory, the specific binding molecule is thought to bind to its target monovalently. The specific binding molecule may be a monovalent binder.

The specific binding molecule may bind to SEQ ID NO: 1 or a fragment thereof with a K_{D} of less than 25 nM, less than 20 nM, less than 15 nM, less than 10 nM, less than 8 nM, less than 6 nM, less than 5 nM, less than 4 nM, less than 3 nM, less than 2 nM, less than 1 nM, less than 0.5 nM, less than 0.4 nM, less than 0.3 nM, less than 0.2 nM or less than 0.15 nM.

The specific binding molecule may bind to SEQ ID NO: 1 or a fragment thereof with a K_{D} of less than 10 nM.

The specific binding molecule may have a K_{D} of 5 to 25 nM. The specific binding molecule may have a K_{D} of 5 to 20 nM. The specific binding molecule may have a K_{D} of 6 to 25 nM. The specific binding molecule may have a K_{D} of 6 to 20 nM.

The specific binding molecule may compete for binding to the same epitope as the epitope bound by mAb423. Without being bound by theory, the epitope of mAb423 is thought to be DHGAE, corresponding to residues 387-391 of SEQ ID NO: 1. It is thought that the binding of mAb423 is Glu-391 specific. Accordingly, mAb423 does not bind to DHGA, corresponding to residues 387-390 of SEQ ID NO: 1. The specific binding molecule may therefore compete for binding to DHGAE, corresponding to residues 387 to 391 of SEQ ID NO: 1. Any specific binding molecule disclosed herein with an epitope comprising one or more of residues 387 to 391 of SEQ ID NO:1 may therefore compete for binding to the same epitope as the epitope bound by mAb423. For example, a specific binding molecule with an epitope within an amino acid sequence comprising residues 369 to 390 of SEQ ID NO: 1 may compete for binding to the same epitope as the epitope bound by mAb423. Preferably, the specific binding molecules that compete for binding to the same epitope as the epitope bound by mAb423 are Glu-391 specific. The specific binding molecule that competes for binding to the same epitope as the epitope bound by mAb423 may therefore be selected from the group consisting of E1E8, E2A6, E2B7, E2E8 and E1B8.

The epitopes of clones directed to the dGAE fragment may include residues thought to be involved in the formation of the “C shaped” architecture of the PHF core (see FIGS. 2 to 4). The binding of such clones may impair the formation of the “C shaped” architecture of the PHF core or inhibit binding of subunits to existing oligomers by steric hinderance and/or inhibiting the association of key residues.

The specific binding molecule may compete with the binding of a first region within residues 296 to 391 of SEQ ID NO: 1 to a second region within residues 296 to 391 of SEQ ID NO: 1. Accordingly, the specific binding molecule may compete with the binding of a first region within the dGAE fragment to a second region within the dGAE fragment.

The specific binding molecule may compete with the binding of a first region within residues 296 to 390 of SEQ ID NO: 1 to a second region within residues 296 to 390 of SEQ ID NO: 1. Accordingly, the specific binding molecule may compete with the binding of a first region within the dGA fragment to a second region within the dGA fragment.

The specific binding molecule may compete with the binding of a first region within residues 308 to 378 of SEQ ID NO: 1 to a second region within residues 308 to 378 of SEQ ID NO: 1. Accordingly, the specific binding molecule may compete with the binding of a first region within dGAE73 and/or dGAE71 to a second region within dGAE73 and/or dGAE71.

The specific binding molecule may compete with the binding of a first region within residues 296 to 386 of SEQ ID NO: 1 to a second region within residues 296 to 386 of SEQ ID NO: 1.

The specific binding molecule may compete with the binding of a first region within residues 306 to 391 of SEQ ID NO: 1 to a second region within residues 306 to 391 of SEQ ID NO: 1.

The specific binding molecule may compete with the binding of a first region within residues 306 to 386 of SEQ ID NO: 1 to a second region within residues 306 to 386 of SEQ ID NO: 1.

The first and second region may be within the same polypeptide molecule. Accordingly, the specific binding molecule may inhibit the formation of a hairpin structure of the PHF core. The specific binding molecule may inhibit the folding of the PHF core.

The PHF core is composed of eight β-sheets (β 1-8) that run along the length of the protofilament, adopting a C-shaped architecture.

Starting from the termini of the PHF, there is a heterotypic cross-β interface formed between β 1-2 and β 8. The N-terminal end of the ordered core is formed by the hexapeptide ₃₀₆VQIVYK₃₁₁, (SEQ ID NO: 430) which forms a complementary packing interface with residues 373-378 from the opposing β 8 by face-to-face packing of hydrophobic groups. The specific binding molecule may compete with the binding of residues 306 to 311 of SEQ ID NO: 1 to residues 373 to 378 of SEQ ID NO: 1. Any specific binding molecule disclosed herein with an epitope overlapping either 306 to 311 of SEQ ID NO: 1 or residues 373 to 378 of SEQ ID NO: 1 may compete with the binding of residues 306 to 311 of SEQ ID NO: 1 to residues 373 to 378 of SEQ ID NO: 1. For example, a specific binding molecule with an epitope within residues 367 to 379 of SEQ ID NO: 1 may compete with the binding of residues 306 to 311 of SEQ ID NO: 1 to residues 373 to 378 of SEQ ID NO: 1. For instance, a specific binding molecule comprising the CDRs of S1G2 (or a derivative thereof) may compete with the binding of residues 306 to 311 of SEQ ID NO: 1 to residues 373 to 378 of SEQ ID NO: 1.

Strands β 2 (residues 313-322 of SEQ ID NO: 1) and β 8 (residues 368-378 of SEQ ID NO: 1) pack against each other through a polar-zipper motif. The specific binding molecule may compete with the binding of residues 313 to 322 of SEQ ID NO: 1 to residues 368 to 378 of SEQ ID NO: 1. Any specific binding molecule disclosed herein with an epitope overlapping either 313 to 322 of SEQ ID NO: 1 or residues 368 to 378 of SEQ ID NO: 1 may compete with the binding of residues 313 to 322 of SEQ ID NO: 1 to residues 368 to 378 of SEQ ID NO: 1. For example, a specific binding molecule with an epitope within residues 367 to 379 of SEQ ID NO: 1 may compete with the binding of residues 313 to 322 of SEQ ID NO: 1 to residues 368 to 378 of SEQ ID NO: 1. For instance, a specific binding molecule comprising the CDRs of S1G2 (or a derivative thereof) may compete with the binding of residues 313 to 322 of SEQ ID NO: 1 to residues 368 to 378 of SEQ ID NO: 1.

A hydrophobic cluster of L324, 1326 and V363 stabilizes the region immediately after a turn in the PHF core and the cross-β interface between β 3 and β 7 is further cemented by hydrogen bonds between the sidechains of H328 and T361. The specific binding molecule may compete with the binding of residues 327 to 331 of SEQ ID NO: 1 to residues 356 to 363 of SEQ ID NO: 1. Any specific binding molecule disclosed herein with an epitope overlapping either 324 to 331 of SEQ ID NO: 1 or residues 356 to 363 of SEQ ID NO: 1 may compete with the binding of residues 327 to 331 of SEQ ID NO: 1 to residues 356 to 363 of SEQ ID NO: 1.

For example, a specific binding molecule with an epitope within residues 319 to 331 of SEQ ID NO: 1 may compete with the binding of residues 327 to 331 of SEQ ID NO: 1 to residues 356 to 363 of SEQ ID NO: 1. For instance, a specific binding molecule comprising the CDRs of CE2 (or a derivative thereof) may compete with the binding of residues 327 to 331 of SEQ ID NO: 1 to residues 356 to 363 of SEQ ID NO: 1.

As a further example, a specific binding molecule with an epitope within residues 355 to 367 of SEQ ID NO: 1 may compete with the binding of residues 327 to 331 of SEQ ID NO: 1 to residues 356 to 363 of SEQ ID NO: 1. For instance, a specific binding molecule comprising the CDRs of CA4 (or a derivative thereof) may compete with the binding of residues 327 to 331 of SEQ ID NO: 1 to residues 356 to 363 of SEQ ID NO: 1.

The two “sides” of the PHF core meet through a β-helix structure that is defined by three β-strands in residues 337 to 368 of SEQ ID NO: 1 (β 4-6). The “hinge” region of the PHF core may be defined as residues 337 to 355 of SEQ ID NO: 1 and may alternatively be defined as the “critical abnormal fold”. Two-residue (E342, K343) and three-residue (₃₄₇KDR₃₄₉) β-arc corners punctuate the triangular β-helix geometry, which is closed with a pivotal ˜70° glycine conformation (G355). Hydrophobic clustering, aliphatic stacking (V339, L344, V350 and I354) and aromatic stacking (F346) stabilize the interior of the β-helix. Any specific binding molecule disclosed herein with an epitope overlapping residues 337 to 355 of SEQ ID NO: 1 may compete with the binding of residues 337 to 355 of SEQ ID NO: 1 to residues 337 to 355 of SEQ ID NO: 1. For example, a specific binding molecule with an epitope within residues 337 to 355 of SEQ ID NO: 1 may compete with the binding of residues 337 to 355 of SEQ ID NO: 1 to residues 337 to 355 of SEQ ID NO: 1. For instance, a specific binding molecule comprising the CDRs of S1D12 (or a derivative thereof) may compete with the binding of residues 337 to 355 of SEQ ID NO: 1 to residues 337 to 355 of SEQ ID NO: 1.

Specific binding molecules competing for binding of regions described above may have utility in preventing the formation of the PHF core structure and therefore inhibiting tau aggregation. A combination of specific binding molecules competing for binding of multiple regions described above may have increased utility in preventing the formation of the PHF core structure and therefore inhibiting tau aggregation.

Alternatively, the first and second region may be within different polypeptide molecules. Accordingly, the specific binding molecule may inhibit the binding of a first polypeptide to a second polypeptide. The first and second polypeptides may comprise the PHF core.

As illustrated in FIGS. 5 and 6, a new dGAE unit progressively unfolds and becomes aligned with the structure of an existing oligomer. This attachment sequence may be understood in terms of 3 stages corresponding to progressive binding of key segments of dGAE and their epitopes into the oligomer. As can be seen, the hinge region recognised by S1D12 is the primary site of attachment, followed by progressive symmetrical binding of the other domains.

The specific binding molecule may compete with the binding of a first polypeptide comprising the amino acid sequence GGGQVEVKSEKLDFKDRVQSK (SEQ ID NO: 375—corresponding to residues 333 to 353 of SEQ ID NO: 1) to a second polypeptide comprising the PHF core. The specific binding molecule may compete with the binding of a first polypeptide comprising residues 333 to 353 of SEQ ID NO: 1 to a second polypeptide comprising residues 333 to 353 of SEQ ID NO: 1. Any specific binding molecule disclosed herein with an epitope overlapping residues 333 to 353 of SEQ ID NO: 1 may compete with the binding of a first polypeptide comprising residues 333 to 353 of SEQ ID NO: 1 to a second polypeptide comprising the PHF core and/or residues 333 to 353 of SEQ ID NO: 1. For example, a specific binding molecule with an epitope within residues 337 to 355 of SEQ ID NO: 1 may compete with the binding of a first polypeptide comprising residues 333 to 353 of SEQ ID NO: 1 to a second polypeptide comprising the PHF core and/or residues 333 to 353 of SEQ ID NO: 1. For instance, a specific binding molecule comprising the CDRs of S1D12 (or a derivative thereof) may compete with the binding of a first polypeptide comprising residues 333 to 353 of SEQ ID NO: 1 to a second polypeptide comprising the PHF core and/or residues 333 to 353 of SEQ ID NO: 1.

The specific binding molecule may compete with the binding of a first polypeptide comprising the amino acid sequence CGSLGNIHHKPG (SEQ ID NO: 376—corresponding to residues 322 to 333 of SEQ ID NO: 1) to a second polypeptide comprising the PHF core. The specific binding molecule may compete with the binding of a first polypeptide comprising residues 322 to 333 of SEQ ID NO: 1 to a second polypeptide comprising residues 322 to 333 of SEQ ID NO: 1. Any specific binding molecule disclosed herein with an epitope overlapping residues 322 to 333 of SEQ ID NO: 1 may compete with the binding of a first polypeptide comprising residues 322 to 333 of SEQ ID NO: 1 to a second polypeptide comprising the PHF core and/or residues 322 to 333 of SEQ ID NO: 1. For example, a specific binding molecule with an epitope within residues 319 to 331 of SEQ ID NO: 1 may compete with the binding of a first polypeptide comprising residues 322 to 333 of SEQ ID NO: 1 to a second polypeptide comprising the PHF core and/or residues 322 to 333 of SEQ ID NO: 1. For instance, a specific binding molecule comprising the CDRs of CE2 (or a derivative thereof) may compete with the binding of a first polypeptide comprising residues 322 to 333 of SEQ ID NO: 1 to a second polypeptide comprising the PHF core and/or residues 322 to 333 of SEQ ID NO: 1.

The specific binding molecule may compete with the binding of a first polypeptide comprising the amino acid sequence SLDNITHVP (SEQ ID NO: 377—corresponding to residues 356 to 364 of SEQ ID NO: 1) to a second polypeptide comprising the PHF core. The specific binding molecule may compete with the binding of a first polypeptide comprising residues 356 to 364 of SEQ ID NO: 1 to a second polypeptide comprising residues 356 to 364 of SEQ ID NO: 1. Any specific binding molecule disclosed herein with an epitope overlapping residues 356 to 364 of SEQ ID NO: 1 may compete with the binding of a first polypeptide comprising residues 356 to 364 of SEQ ID NO: 1 to a second polypeptide comprising the PHF core and/or residues 356 to 364 of SEQ ID NO: 1. For example, a specific binding molecule with an epitope within residues 355 to 367 of SEQ ID NO: 1 may compete with the binding of a first polypeptide comprising residues 356 to 364 of SEQ ID NO: 1 to a second polypeptide comprising the PHF core and/or residues 356 to 364 of SEQ ID NO: 1. For instance, a specific binding molecule comprising the CDRs of CA4 (or a derivative thereof) may compete with the binding of a first polypeptide comprising residues 356 to 364 of SEQ ID NO: 1 to a second polypeptide comprising the PHF core and/or residues 356 to 364 of SEQ ID NO: 1.

The specific binding molecule may compete with the binding of a first polypeptide comprising the amino acid sequence VQIVYKPVD (SEQ ID NO: 378—corresponding to residues 306 to 314 of SEQ ID NO: 1) to a second polypeptide comprising the PHF core. The specific binding molecule may compete with the binding of a first polypeptide comprising residues 306 to 314 of SEQ ID NO: 1 to a second polypeptide comprising residues 306 to 314 of SEQ ID NO: 1. Any specific binding molecule disclosed herein with an epitope overlapping residues 306 to 314 of SEQ ID NO: 1 may compete with the binding of a first polypeptide comprising residues 306 to 314 of SEQ ID NO: 1 to a second polypeptide comprising the PHF core and/or residues 306 to 314 of SEQ ID NO: 1. For example, a specific binding molecule with an epitope comprising residues 306 to 314 of SEQ ID NO: 1 may compete with the binding of a first polypeptide comprising residues 306 to 314 of SEQ ID NO: 1 to a second polypeptide comprising the PHF core and/or residues 306 to 314 of SEQ ID NO: 1.

The specific binding molecule may compete with the binding of a first polypeptide comprising the amino acid sequence KKIETHKLTF (SEQ ID NO: 379—corresponding to residues 369 to 378 of SEQ ID NO: 1) to a second polypeptide comprising the PHF core. The specific binding molecule may compete with the binding of a first polypeptide comprising residues 369 to 378 of SEQ ID NO: 1 to a second polypeptide comprising residues 369 to 378 of SEQ ID NO: 1. Any specific binding molecule disclosed herein with an epitope overlapping residues 369 to 378 of SEQ ID NO: 1 may compete with the binding of a first polypeptide comprising residues 369 to 378 of SEQ ID NO: 1 to a second polypeptide comprising the PHF core and/or residues 369 to 378 of SEQ ID NO: 1. For example, a specific binding molecule with an epitope within residues 367 to 379 of SEQ ID NO: 1 may compete with the binding of a first polypeptide comprising residues 369 to 378 of SEQ ID NO: 1 to a second polypeptide comprising the PHF core and/or residues 369 to 378 of SEQ ID NO: 1. For instance, a specific binding molecule comprising the CDRs of S1G2 (or a derivative thereof) may compete with the binding of a first polypeptide comprising residues 369 to 378 of SEQ ID NO: 1 to a second polypeptide comprising the PHF core and/or residues 369 to 378 of SEQ ID NO: 1.

Without being bound by theory, in any embodiment where the binding of a specific binding molecule competes with the binding of a first region within residues 296 to 391 of SEQ ID NO: 1 to a second region within residues 296 to 391 of SEQ ID NO: 1 (or within the other ranges or residues of SEQ ID NO: 1 set out above), the specific binding molecule may inhibit the tau aggregation. This applies whether the first and second region are within the same or different polypeptide molecules. Any suitable test for determining competitive binding or for screening inhibitors of tau aggregation may therefore be used to confirm that a specific binding molecule competes with the binding of a first region within residues 296 to 391 of SEQ ID NO: 1 to a second region within residues 296 to 391 of SEQ ID NO: 1. Suitable screening methods include a thioflavin T-assay, a tau-tau immunoassay and an assay for assessing the effects of aggregated tau in cell culture. A suitable assay for assessing the effects of aggregated tau in cell culture is disclosed in UK application no. GB2010620.9 filed on 10 Jul. 2020, and in international (PCT) application no. PCT/EP2021/069138 filed on 9 Jul. 2021 and claiming priority to UK application no. GB2010620.9, both of which are hereby incorporated by reference in their entirety. The invention relates to specific binding molecules with high affinities for their ligands. A specific binding molecule with high affinity for its ligand is advantageous in the present invention, as, generally, less of a specific binding molecule with high affinity for its ligand is required to achieve a particular effect than of a specific binding molecule with lower affinity for the same ligand. For instance, if the specific binding molecule is for therapeutic use, it can be expected that a lower dosage would be required of a specific binding molecule with high affinity for its ligand than of a specific binding molecule with lower affinity for the same ligand. This may be advantageous for the patient, who might require fewer or smaller doses of the specific binding molecule, e.g. antibody, and would also be more economical, as less of the specific binding molecule would be required for the therapy.

The affinity of a binding molecule for its ligand (or binding partner), such as the affinity of an antibody for its target antigen, can be quantitatively defined by the dissociation constant (K_{D}) for a complex of the binding molecule and ligand. The K_{D} value of a specific binding molecule, e.g. an antibody, corresponds to the ratio of the binding molecule dissociation rate (i.e. how quickly it dissociates from its ligand) to the binding molecule association rate (i.e. how quickly it binds its ligand). A lower K_{D} value corresponds to a higher binding affinity of the binding molecule for its ligand. K_{D} may be measured under any suitable conditions for binding of specific binding molecule to its ligand, preferably under conditions identified as optimal. Methods as described in the Examples may be used. Alternatively, any other conditions identified as promoting the binding of the specific binding molecule to a peptide comprising the epitope within SEQ ID NO: 1 bound by the specific binding molecule may be used. A number of methods by which the K_{D} of an interaction between a specific binding molecule and its ligand may be calculated are well-known in the art. Known techniques include SPR (e.g. Biacore) and polarization-modulated oblique-incidence reflectivity difference (OI-RD).

The specific binding molecule may be an isolated specific binding molecule.

As indicated, the specific binding molecule comprises 6 CDRs consisting of polypeptide sequences. As used herein, “protein” and “polypeptide” are interchangeable, and each refer to a sequence of 2 or more amino acids joined by one or more peptide bonds. Thus, the specific binding molecule may be a polypeptide. Alternatively, the specific binding molecule may comprise one or more polypeptides which comprise the CDR sequences. Preferably, the specific binding molecule is an antibody or an antibody fragment.

When a CDR sequence is modified by substitution of a particular amino acid residue, the substitution may be a conservative amino acid substitution. However, a substitution of a CDR residue may equally be a non-conservative substitution, in which one amino acid is substituted for another with a side-chain belonging to a different family.

Wherever the present application describes a CDR sequence comprising one, two, or three amino acid substitutions relative to a specified CDR sequence, said one, two, or three amino acid substitutions may be conservative amino acid substitutions. Preferably the CDR sequence comprises two conservative amino acid substitutions. More preferably the CDR sequence comprises one conservative amino acid substitution.

When a FR sequence is modified by substitution of a particular amino acid residue, the substitution may be a conservative amino acid substitution. However, a substitution of a FR residue may equally be a non-conservative substitution, in which one amino acid is substituted for another with a side-chain belonging to a different family.

Wherever the present application describes a FR sequence comprising one, two, three, four or five amino acid substitutions relative to a specified FR sequence, said one, two, three, four or five amino acid substitutions may be conservative amino acid substitutions. Preferably the FR sequence comprises four conservative amino acid substitutions. Preferably the FR sequence comprises three conservative amino acid substitutions. Preferably the FR sequence comprises two conservative amino acid substitutions. More preferably the FR sequence comprises one conservative amino acid substitution.

Amino acid substitutions or additions in the scope of the invention may be made using a proteinogenic amino acid encoded by the genetic code, a proteinogenic amino acid not encoded by the genetic code, or a non-proteinogenic amino acid. Preferably any amino acid substitution or addition is made using a proteinogenic amino acid. The amino acids making up the sequence of the CDRs may include amino acids which do not occur naturally, but which are modifications of amino acids which occur naturally. Providing these non-naturally occurring amino acids do not alter the sequence and do not affect specificity, they may be used to generate CDRs described herein without reducing sequence identity, i.e. are considered to provide an amino acid of the CDR. For example derivatives of the amino acids such as methylated amino acids may be used. The specific binding molecule may be a non-natural molecule, i.e. not a molecule found in nature.

Modifications to the amino acid sequences of the CDRs set out in herein may be made using any suitable technique, such as site-directed mutagenesis of the encoding DNA sequence or solid state synthesis.

Specific binding molecules disclosed herein comprise CDRs as described herein. Additionally, such molecules may contain linker moieties or framework sequences to allow appropriate presentation of the CDRs. Additional sequences may also be present which may conveniently confer additional properties, e.g. peptide sequences which allow isolation or identification of the molecules containing the CDRs such as those described hereinbefore. In such cases a fusion protein may be generated.

The CDRs of the specific binding molecule may be defined as having a certain percentage sequence identity to one or more SEQ ID NOs described herein. Sequence identity may be assessed by any convenient method. However, for determining the degree of sequence identity between sequences, computer programmes that make pairwise or multiple alignments of sequences are useful, for instance EMBOSS Needle or EMBOSS stretcher (both Rice, P. et al., Trends Genet., 16, (6) pp 276-277, 2000) may be used for pairwise sequence alignments while Clustal Omega (Sievers F et al., Mol. Syst. Biol. 7:539, 2011) or MUSCLE (Edgar, R. C., Nucleic Acids Res. 32 (5): 1792-1797, 2004) may be used for multiple sequence alignments, though any other appropriate programme may be used. Whether the alignment is pairwise or multiple, it must be performed globally (i.e. across the entirety of the reference sequence) rather than locally.

Sequence alignments and % identity calculations may be determined using for instance standard Clustal Omega parameters: matrix Gonnet, gap opening penalty 6, gap extension penalty 1. Alternatively, the standard EMBOSS Needle parameters may be used: matrix BLOSUM62, gap opening penalty 10, gap extension penalty 0.5. Any other suitable parameters may alternatively be used.

For the purposes of this application, where there is dispute between sequence identity values obtained by different methods, the value obtained by global pairwise alignment using EMBOSS Needle with default parameters shall be considered valid.

Wherever the disclosure provides a CDR sequence with at least 85% identity to a specified CDR sequence, said sequence identity is at least about 85% sequence identity and may therefore be at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity. Preferably said sequence identity is at least 90% or at least 95%.

As stated above, the specific binding molecule is preferably an antibody or an antibody fragment. An “antibody” is an immunoglobulin having the features described hereinbefore. Also contemplated are variants of naturally occurring antibodies which retain the CDRs but are presented in a different framework, as discussed hereinafter and which function in the same way, i.e. retain specificity for the antigen. Thus, antibodies include functional equivalents or homologues in which naturally occurring domains have been replaced in part or in full with natural or non-natural equivalents or homologues which function in the same way.

The first and/or second specific binding molecule may be an immunoglobin, an immunoglobin Fab region, a Fab′, a Fv, a Fv-Fc, a single chain Fv (scFv), scFv-Fc, (scFv)₂, a diabody, a triabody, a tetrabody, a bispecific t-cell engager (BiTE), an intein, a VNAR domain, a single domain antibody (sdAb) or a VH domain.

When the specific binding molecule is an antibody, it is preferably a monoclonal antibody. By “monoclonal antibody” is meant an antibody preparation consisting of a single antibody species, i.e. all antibodies in the preparation have the same amino acid sequences, including the same CDRs, and thus bind the same epitope on their target antigen (by “target antigen” is meant the antigen containing the epitope bound by a particular antibody, i.e. the target antigen of an anti-2N4R antibody is 2N4R) with the same effect. In other words, the antibody is preferably not part of a polyclonal mix of antibodies.

In an antibody, as described above, the CDR sequences are located in the variable domains of the heavy and light chains. The CDR sequences sit within a polypeptide framework, which positions the CDRs appropriately for antigen binding. Thus, the remainder of the variable domains (i.e. the parts of the variable domain sequences which do not form a part of any one of the CDRs) constitute framework regions. The N-terminus of a mature variable domain forms framework region 1 (FR1); the polypeptide sequence between CDR1 and CDR2 forms FR2; the polypeptide sequence between CDR2 and CDR3 forms FR3; and the polypeptide sequence linking CDR3 to the constant domain forms FR4. In an antibody the variable region framework regions may have any appropriate amino acid sequence such that the antibody binds to SEQ ID NO: 1 or a fragment thereof via its CDRs. The constant regions may be the constant regions of any mammalian (preferably human) antibody isotype.

In certain embodiments of the invention the specific binding molecule may be multi-specific, e.g. a bi-specific monoclonal antibody. A multi-specific binding molecule contains regions or domains (antigen-binding regions) which bind to at least two different molecular binding partners, e.g. bind to two or more different antigens or epitopes. In the case of a bi-specific antibody, the antibody comprises two heavy and light chains, in the formation as described above, except that the variable domains of the two heavy chains and the two light chains, respectively, are different, and thus form two different antigen-binding regions. In a multi-specific (e.g. bi-specific) binding molecule, e.g. monoclonal antibody, one of the antigen-binding regions has the CDR sequences of a specific binding molecule as defined herein, and thus binds SEQ ID NO: 1 or a fragment thereof. The other antigen-binding region(s) of the multi-specific binding molecule are different to the antigen-binding regions formed by CDRs, e.g. have CDRs with sequences different to those defined herein for the specific binding molecule. The additional (e.g. second) antigen-binding region(s) of the specific binding molecule, e.g. in the bi-specific antibody, may also bind SEQ ID NO: 1 or a fragment thereof, but at a different epitope to the first antigen-binding region which binds to SEQ ID NO: 1 or a fragment thereof (which has the CDRs of the specific binding molecule). Alternatively, the additional (e.g. second) antigen-binding region(s) may bind additional (e.g. a second), different antigen(s) which is (are) not SEQ ID NO: 1 or a fragment thereof. In an alternative embodiment, the two or more antigen-binding regions in the specific binding molecule, e.g. in an antibody, may each bind to the same antigen, i.e. provide a multivalent (e.g. bivalent) molecule.

The specific binding molecule may be an antibody fragment or synthetic construct capable of binding human SEQ ID NO: 1 or a fragment thereof. Antibody fragments are discussed in Rodrigo et al., Antibodies, Vol. 4(3), p. 259-277, 2015. Antibody fragments are preferably monoclonal (i.e. they are not part of a polyclonal mix of antibody fragments). Antibody fragments include, for example, Fab, F(ab′)₂, Fab′ and Fv fragments. Fab fragments are discussed in Roitt et al, Immunology second edition (1989), Churchill Livingstone, London. A Fab fragment consists of the antigen-binding domain of an antibody, i.e. an individual antibody may be seen to contain two Fab fragments, each consisting of a light chain and its conjoined N-terminal section of the heavy chain. Thus, a Fab fragment contains an entire light chain and the VH and CH1 domains of the heavy chain to which it is bound. Fab fragments may be obtained by digesting an antibody with papain.

F(ab′)₂ fragments consist of the two Fab fragments of an antibody, plus the hinge regions of the heavy domains, including the disulphide bonds linking the two heavy chains together. In other words, a F(ab′)₂ fragment can be seen as two covalently joined Fab fragments. F(ab′)₂ fragments may be obtained by digesting an antibody with pepsin. Reduction of F(ab′)₂ fragments yields two Fab′ fragments, which can be seen as Fab fragments containing an additional sulfhydryl group which can be useful for conjugation of the fragment to other molecules.

Fv fragments consist of just the variable domains of the light and heavy chains. These are not covalently linked and are held together only weakly by non-covalent interactions. Fv fragments can be modified to produce a synthetic construct known as a single chain Fv (scFv) molecule. Such a modification is typically performed recombinantly, by engineering the antibody gene to produce a fusion protein in which a single polypeptide comprises both the VH and VL domains. scFv fragments generally include a peptide linker covalently joining the VH and VL regions, which contributes to the stability of the molecule. The linker may comprise from 1 to 20 amino acids, such as for example 1, 2, 3 or 4 amino acids, 5, 10 or 15 amino acids, or other intermediate numbers in the range 1 to 20 as convenient. The peptide linker may be formed from any generally convenient amino acid residues, such as glycine and/or serine. One example of a suitable linker is Gly₄Ser. Multimers of such linkers may be used, such as for example a dimer, a trimer, a tetramer or a pentamer, e.g. (Gly₄Ser)₂, (Gly₄Ser)₃, (Gly₄Ser)₄ or (Gly₄Ser)₅. However, it is not essential that a linker be present, and the VL domain may be linked to the VH domain by a peptide bond. An scFv is herein defined as an antibody fragment.

The specific binding molecule may be an analogue of an scFv. For example, the scFv may be linked to other specific binding molecules (for example other scFvs, Fab antibody fragments and chimeric IgG antibodies (e.g. with human frameworks)). The scFv may be linked to other scFvs so as to form a multimer which is a multi-specific binding protein, for example a dimer, a trimer or a tetramer. Bi-specific scFvs are sometimes referred to as diabodies, tri-specific scFvs as triabodies and tetra-specific scFvs as tetrabodies. In other embodiments the scFv may be bound to other, identical scFv molecules, thus forming a multimer which is mono-specific but multi-valent, e.g. a bivalent dimer or a trivalent trimer may be formed. Synthetic constructs that can be used include CDR peptides. These are synthetic peptides comprising antigen-binding determinants. Peptide mimetics can also be used. These molecules are usually conformationally-restricted organic rings that mimic the structure of a CDR loop and that include antigen-interactive side chains.

The specific binding molecule may be a scAb (single-chain antibody). A scAb may comprise an scFv. The scFv may comprise a variable heavy domain and a variable light domain optionally joined by a flexible protein linker as described above. A scAb may further comprise a light chain constant domain. The light chain constant domain may be human, such as a human Ck domain.

The antibody or antibody fragment may be a chimeric antibody, or preferably may be humanised. This is particularly the case for monoclonal antibodies and antibody fragments. Humanised or chimeric antibodies or antibody fragments are desirable when the molecule is to be used as a human therapeutic. Therapeutic treatment of humans with murine antibodies can be ineffective for a number of reasons, e.g. a short in vivo half-life of the antibody; weak effector functions mediated by the mouse heavy chain constant region due to low recognition of the murine heavy chain constant region by Fc receptors on human immune effector cells; patient sensitisation to the antibody, and generation of a human anti-mouse antibody (HAMA) response; and neutralisation of the mouse antibody by HAMA leading to loss of therapeutic efficacy.

As detailed above, the isotype of an antibody is defined by the sequence of its heavy chain constant regions. The chimeric antibody may have the constant regions of any human antibody isotype, and any sub-class within each isotype. For instance, the chimeric antibody may have the Fc regions of an IgA, IgD, IgE, IgG or IgM antibody (i.e. the chimeric antibody may comprise the constant domains of heavy chains α, δ, ε, γ, or μ, respectively), though preferably the antibody is of the IgG isotype. Thus, the chimeric antibody may be of any isotype. The light chain of the chimeric antibody may be either a κ or λ light chain, i.e. it may comprise the constant region of a human λ light chain or a human κ light chain. A chimeric antibody fragment is, correspondingly, an antibody fragment comprising constant domains (e.g. an Fab, Fab′ or F(ab′)₂ fragment). The constant domains of a chimeric antibody fragment may be as described above for a chimeric monoclonal antibody.

Chimeric antibodies may be generated using any suitable technique, e.g. recombinant DNA technology in which the DNA sequence of the murine variable domain is fused to the DNA sequence of the human constant domain(s) so as to encode a chimeric antibody. A chimeric antibody fragment may be obtained either by using recombinant DNA technology to produce a DNA sequence encoding such a polypeptide, or by processing a chimeric antibody to produce the desired fragments, as described above. Chimeric antibodies can be expected to overcome the problems of a short in vivo half-life and weak effector functions associated with using a murine antibody in human therapy, and may reduce the probability of patient sensitisation and HAMA occurring. However, patient sensitisation and HAMA may still occur when a chimeric antibody is administered to a human patient, due to the presence of murine sequences in the variable domains.

Preferably the antibody or antibody fragment is therefore fully humanised. A humanised antibody is an antibody derived from another species, e.g. a mouse, in which not only are the constant domains of the antibody chains replaced with human constant domains, but the amino acid sequences of the variable regions are modified, in particular to replace the foreign (e.g. murine) framework sequences with human framework sequences, such that, preferably, the only non-human sequences in the antibody are the CDR sequences. A humanised antibody can overcome all the problems associated with therapeutic use of a non-human antibody in a human, including avoiding or minimising the probability of patient sensitisation and HAMA occurring.

Antibody humanisation is generally performed by a process known as CDR grafting, though any other technique in the art may be used. CDR grafting is well described in Williams, D. G. et al., Antibody Engineering Vol. 1, edited by R. Kontermann and S. Dübel, Chapter 21, pp. 319-339. In this process, a chimeric antibody as described above is first generated. Subsequent humanisation of the foreign, e.g. murine, variable domains involves intercalating the murine CDRs from each immunoglobulin chain within the FRs of the most appropriate human variable region. This is done by aligning the murine variable domains with databases of known human variable domains (e.g. IMGT or Kabat). Appropriate human framework regions are identified from the best aligned variable domains, e.g. domains with high sequence identity between the human and murine framework regions, domains containing CDRs of the same length, domains having the most similar structures (based on homology modelling), etc. The murine CDR sequences are then grafted into the lead human framework sequences at the appropriate locations using recombinant DNA technology, and the humanised antibodies then produced and tested for binding to the target antigen. The process of antibody humanisation is known and understood by the skilled individual, who can perform the technique without further instruction. Antibody humanisation services are also offered by a number of commercial companies, e.g. GenScript (USA/China) or MRC Technology (UK). Humanised antibody fragments can be easily obtained from humanised antibodies, as described above.

Thus, the antibody or antibody fragment may be derived from any species, e.g. it may be a murine antibody or antibody fragment. It is preferred, however, that the antibody or antibody fragment is a chimeric antibody or antibody fragment, i.e. that only the variable domains of the antibody or antibody fragment are non-human, and the constant domains are all human. Optimally, the antibody or antibody fragment is a humanised antibody or antibody fragment.

### Tauopathies

Aggregation of the tau protein is a hallmark of diseases referred to as “tauopathies”. Various tauopathy disorders that have been recognized which feature prominent tau pathology in neurons and/or glia and this term has been used in the art for several years. The similarities between these pathological inclusions and the characteristic tau inclusions in diseases such as AD indicate that the structural features are shared and that it is the topographic distribution of the pathology that is responsible for the different clinical phenotypes observed. In particular, cryo-electron microscope structures of aggregated Tau in AD, Pick's disease (a subtype of Frontotemporal Dementia), chronic traumatic encephalopathy (CTE) and cortico-basal degeneration (CBD) have been obtained previously, and all show common conformational features, indicating that compounds that have the ability to modulate Tau aggregation in e.g. PHFs (as observed in AD), may also modulate aggregation of Tau in other tauopathies. In addition to specific diseases discussed below, those skilled in the art can identify tauopathies by combinations of cognitive or behavioural symptoms, plus additionally through the use of appropriate ligands for aggregated tau as visualised using PET or MRI, such as those described in WO02/075318.

Aspects of the present invention relate to “tauopathies”. As well as Alzheimer's disease (AD), the pathogenesis of neurodegenerative disorders such as Pick's disease and Progressive Supranuclear Palsy (PSP) appears to correlate with an accumulation of pathological truncated tau aggregates in the dentate gyrus and stellate pyramidal cells of the neocortex, respectively. Relevant dementias include fronto-temporal dementia (FTD); behavioral frontotemporal dementia (bvFTD), frontotemporal dementia with parkinsonism linked to chromosome 17 (FTDP-17); disinhibition-dementia-parkinsonism-amyotrophy complex (DDPAC); pallido-ponto-nigral degeneration (PPND); Guam-ALS syndrome; pallido-nigro-luysian degeneration (PNLD); cortico-basal degeneration (CBD); Dementia with Argyrophilic grains (AgD); Dementia pugilistica (DP) wherein despite different topography, NFTs are similar to those observed in AD (Bouras et al., 1992); Chronic traumatic encephalopathy (CTE), a tauopathy including DP as well as repeated and sports-related concussion (McKee, et al., 2009). Others are discussed in Wischik et al. 2000, for detailed discussion—especially Table 5.1).

Abnormal tau in NFTs is found also in Down's Syndrome (DS) (Flament et al., 1990), and in dementia with Lewy bodies (DLB) (Harrington et al., 1994). Tau-positive NFTs are also found in Postencephalitic parkinsonism (PEP) (Charpiot et al., 1992). Glial tau tangles are observed in Subacute sclerosing panencephalitis (SSPE) (Ikeda et al., 1995). Other tauopathies include Niemann-Pick disease type C (NPC) (Love et al., 1995); Sanfilippo syndrome type B (or mucopolysaccharidosis III B, MPS III B) (Ohmi, et al., 2009); myotonic dystrophies (DM), DM1 (Sergeant, et al., 2001 and references cited therein) and DM2 (Maurage et al., 2005). Additionally, there is a growing consensus in the literature that a tau pathology may also contribute more generally to cognitive deficits and decline, including in mild cognitive impairment (MCI) (see e.g. Braak, et al., 2003, Wischik et al., 2018).

All of these diseases, which are characterized primarily or partially by abnormal tau aggregation, are referred to herein as “tauopathies” or “diseases of tau protein aggregation”. In aspects of the invention relating to tauopathies, the tauopathy may be selected from any tauopathy defined herein. Without wishing to be bound by theory, the present inventors believe that all structures solved for tauopathies encompass the dGAE region of Tau. As such, specific binding molecules that stabilise a conformation of dGAE that is not prone to assembly by binding to dGAE can reasonably be expected to apply to all tau diseases including but not limited to AD.

The tauopathy may be selected from the group consisting of Alzheimer's disease, Primary age-related tauopathy (PART), Neurofibrillary tangle-predominant senile dementia, Chronic traumatic encephalopathy (CTE), Progressive supranuclear palsy (PSP), Corticobasal degeneration (CBD), Frontotemporal dementia (FTD), behavioral variant frontotemporal dementia (bvFTD), Frontotemporal dementia with parkinsonism linked to chromosome 17 (FTDP-17), Pick disease, disinhibition-dementia-parkinsonism-amyotrophy complex (DDPAC), pallido-ponto-nigral degeneration (PPND), Guam-ALS syndrome; pallido-nigro-luysian degeneration (PNLD), Dementia with Argyrophilic grains (AgD), Down's Syndrome (DS), dementia with Lewy bodies (DLB), Postencephalitic parkinsonism (PEP), Dementia pugilistica (DP), traumatic brain injury (TBI), stroke, ischemia, Lytico-bodig disease (Parkinson-dementia complex of Guam), Ganglioglioma, Gangliocytoma, Meningioangiomatosis, Postencephalitic parkinsonism, Subacute sclerosing panencephalitis (SSPE), Lead encephalopathy, tuberous sclerosis, Pantothenate kinase-associated neurodegeneration, lipofuscinosis and mild cognitive impairment (MCI).

The tauopathy may be selected from the group consisting of Alzheimer's disease, Frontotemporal dementia (FTD), behavioral variant FTD (bvFTD) and mild cognitive impairment (MCI).

The tauopathy may be Alzheimer's disease.

Tau protein fragments include SEQ ID NO: 3, SEQ ID NO: 4, SEQ ID NO: 5, SEQ ID NO: 6 and SEQ ID NO: 7.

By “contacting” is meant “exposing to”. It does not require directionality; the tau protein or a fragment thereof may be exposed to the specific binding molecule or the specific binding molecule may be exposed to the tau protein or a fragment thereof. The contacting occurs under conditions permissive for binding of the specific binding molecule to the tau protein or fragment thereof.

### Samples

The sample may be defined as a “patient sample” or a “biological sample”. The sample may be from a subject suffering from or at risk of a tauopathy. The sample may be from any subject, including an asymptomatic subject. The sample may be from a human.

Samples are typically obtained prior to the methods of the invention being performed. The methods of the invention are in vitro or ex vivo methods accordingly. In some alternative embodiments, the method may further comprise a step or steps of sample collection.

The sample is a saliva sample. The sample may be unstimulated saliva. The sample may be whole saliva or saliva supernatant. Preferably, the sample is saliva supernatant. The sample may be from a subject who has fasted for at least 15 minutes, at least 30 minutes, at least 1 hour, at least 2 hours or at least 5 hours prior to sample collection. The sample may be from a subject who has fasted for at least 30 minutes prior to sample collection. The sample may be from a subject who washed their mouth out prior to sample collection, for example with water. The sample may have been collected in at least 0.5 ml, at least 1 ml, at least 1.5 ml, at least 2 ml or at least 5 ml of saliva. The sample may have been collected in at least 1 ml of saliva. The sample may have been centrifuged after collection. For example, the sample may have been centrifuged at around 10,000×g for around 5 min or any other suitable centrifugation parameter to separate the saliva supernatant from any debris. The sample may have been stored under any suitable conditions, for example the sample may have been aliquoted and/or stored at −80° C.

The sample may be processed in any suitable way prior to detecting the tau protein or a fragment thereof in a sample. The tau protein or fragment thereof may be isolated, extracted and/or purified from the sample. The isolation, extraction and/or purification may be performed by any suitable technique.

The method of the invention may further comprise an initial step of isolating, extracting and/or purifying the tau protein or fragment thereof from the sample. The method may therefore further comprise isolating the tau protein or fragment thereof from the sample. The method may further comprise extracting the tau protein or fragment thereof from the sample. The method may further comprise purifying the tau protein or fragment thereof from the sample.

The step of obtaining the sample and/or the step of isolating, extracting and/or purifying the tau protein or fragment thereof from the sample may occur in a different location to the subsequent steps of the method. Accordingly, the method may further comprise a step of transporting the sample and/or transporting the tau protein or fragment thereof.

The sample (the term “sample” includes the tau protein or fragment thereof isolated and/or purified therefrom) may be denatured, for example by treatment with sodium dodecyl sulphate (SDS). The method may further comprise a step of denaturing the tau protein or fragment thereof prior to contacting the sample with the specific binding molecule. The denaturing may preferably be by contacting the sample with sodium dodecyl sulphate (SDS). The sample may therefore comprise denatured protein. The tau protein or a fragment thereof may be denatured.

### Detection

The term “detecting” as used herein encompasses quantitative or qualitative detection. “Detecting” may include measuring and/or quantifying the amount (or level) of a tau protein or a fragment thereof in a sample.

The tau protein or fragment thereof may be detected using an immunoassay. Immunoassays have the potential to be miniaturised to run on a microfluidics device or test-strip and may be more suited for clinical point-of-care applications. Embodiments of the invention which incorporate an immunoassay may therefore be used in situ by a primary healthcare provider for assistance in prescribing a treatment for an individual patient.

The amount (or level) of a tau protein or a fragment thereof in a sample may be measured using a homogeneous or heterogeneous immunoassay.

Thus, in some embodiments, the amount (or level) of a tau protein or a fragment thereof may be measured in solution by binding to specific binding molecules of the invention that are present in excess, whereby binding alters detectable properties of the label. The amount of tau protein or a fragment thereof present will therefore affect the amount of the label with a particular detectable property. As is well known in the art, the label may comprise a radioactive label, a fluorescent label or an enzyme having a chromogenic or chemiluminescent substrate that is coloured or caused or allowed to fluoresce when acted on by the enzyme.

Alternatively, a heterogeneous format may be used in which the at least one tau protein or a fragment thereof is captured by surface-bound antibodies for separation and quantification. In some embodiments, a sandwich assay may be used in which a surface-bound tau protein or a fragment thereof is quantified by binding a labelled secondary antibody.

Suitably, the immunoassay may comprise an enzyme immunoassay (EIA) in which the label is an enzyme such, for example, as horseradish peroxidase (HRP). Suitable substrates for HRP are well known in the art and include, for example, ABTS, OPD, AmplexRed, DAB, AEC, TMB, homovanillic acid and luminol. In some embodiments, an ELISA immunoassay may be used; a sandwich ELISA assay may be particularly preferred.

The immunoassay may be competitive or non-competitive. Thus, in some embodiments, the amount of a tau protein or a fragment thereof may be measured directly by a homogeneous or heterogeneous method, as described above. Alternatively, the amount of a tau protein or a fragment thereof in the sample may be sequestered in solution with a specific binding molecule which is present in excess, and the amount of specific binding molecule remaining then determined by binding to surface-bound tau protein or a fragment thereof to give an indirect read-out of the amount of tau protein or a fragment thereof in the original sample. In another variant, the tau protein or a fragment thereof may be caused to compete for binding to a surface bound specific binding molecule with a known amount of a labelled tau protein or a fragment thereof.

The surface bound specific binding molecule or tau protein or a fragment thereof may be immobilised on any suitable surface of the kind known in the art. For instance, the specific binding molecule or tau protein or a fragment thereof may be immobilised on a surface of a well or plate or on the surface of a plurality of magnetic or non-magnetic beads.

In some embodiments, the immunoassay may be a competitive assay, further comprising a known amount of the tau protein or a fragment thereof, which is the same as the one to be quantified in the sample, but tagged with a detectable label. The labelled tau protein or a fragment thereof may be affinity-bound to a suitable surface by a specific binding molecule to the tau protein or a fragment thereof. Upon adding the sample, a proportion of the labelled tau protein or a fragment thereof may be displaced from the surface-bound specific binding molecule, thereby providing a measure of the level of tau protein or a fragment thereof in the sample.

In some embodiments, the immunoassay may comprise surface-bound tau protein or a fragment thereof, which is the same as the tau protein or a fragment thereof that is to be quantitated in the sample, and a known amount of specific binding molecule to the tau protein or a fragment thereof in solution in excess. The sample is first mixed with the specific binding molecule in solution such that a proportion of the specific binding molecules bind with the tau protein or a fragment thereof in the sample. The amount of unbound specific binding molecules remaining can then be measured by binding to the surface-bound tau protein or a fragment thereof.

In some embodiments, the immunoassay may comprise a labelled secondary antibody to the tau protein or a fragment thereof or to a primary antibody to the tau protein or a fragment thereof for quantifying the amount of the tau protein or a fragment thereof bound to surface-bound antibodies or the amount of primary antibody bound to the tau protein or a fragment thereof immobilised on a surface.

Measuring the level of a tau protein or a fragment thereof may be by equipment for measuring the level of a tau protein or a fragment thereof in a sample comprising a sample collection device and an immunoassay. The equipment may further comprise a detector for detecting labelled tau protein or a fragment thereof or labelled antibodies to the tau protein or a fragment thereof in the immunoassay. Suitable labels are mentioned above, but in a preferred embodiment, the label may be an enzyme having a chromogenic or chemiluminescent substrate that is coloured or caused or allowed to fluoresce when acted on by the enzyme.

The immunoassay or equipment may be incorporated into a miniaturised device for measuring the level of a tau protein or a fragment thereof in a biological sample. Suitably, the device may comprise a lab-on-a-chip.

Measuring levels of tau protein or a fragment thereof may be by a device for measuring the level of at least one tau protein or a fragment thereof in a sample obtained from a patient, the device comprising one or more parts defining an internal channel having an inlet port and a reaction zone, in which a tau protein or a fragment thereof in a sample may be reacted with an immobilised primary antibody for the tau protein or a fragment thereof for capturing the tau protein or a fragment thereof, or a primary antibody for the tau protein or a fragment thereof in excess in solution after mixing with the sample upstream of the reaction zone may be reacted with tau protein or a fragment thereof, which is the same as the one to be measured in the sample, but immobilised on a surface within the reaction zone, for quantifying directly or indirectly the amount of the tau protein or a fragment thereof in the sample.

The captured tau protein or a fragment thereof or primary antibody may then be detected using a secondary antibody to the tau protein or a fragment thereof or primary antibody, which is tagged with an enzyme.

As described above, the enzyme may have a chromogenic or chemiluminescent substrate that is coloured or caused or allowed to fluoresce when acted on by the enzyme. Suitably, the one or more parts of the device defining the channel, at least adjacent the reaction zone, may be transparent to light, at least in a range of wavelengths encompassing the colour or fluorescence of the substrate to allow detection of a reaction between the tau protein or a fragment thereof or primary antibody and the secondary antibody using a suitable detector such, for example, as a photodiode, positioned outside the channel or further channel.

In some embodiments, the device may comprise a plurality of channels, each with its own inlet port, for measuring the levels of a plurality of different tau protein or a fragments thereof in the sample in parallel. Therefore, each channel may include a different respective immobilised primary antibody or tau protein or a fragment thereof.

Suitably, the device may comprise one or more selectively operable valves associated with the one or more inlet ports for controlling the admission of a sequence of different reagents into to the channels such, for example, as the sample, wash solutions, primary antibody, secondary antibody and enzyme substrate.

The device therefore may comprise a microfluidics device. The channel may include a reaction zone. Microfluidics devices are known to those skilled in the art. A review of microfluidic immunoassays or protein diagnostic chip microarrays is provided by Chin et al. 2012. Lab on a Chip. 2012; 12:2118-2134. A microfluidics device suitable for carrying out an ELISA immunoassay at a point-of-care is disclosed by Chan C D, Laksanasopin T, Cheung Y K, Steinmiller D et al. “Microfluidics-based diagnostics of infectious diseases in the developing world”. Nature Medicine. 2011; 17 (8): 1015-1019, the contents of which are incorporated herein by reference.

The tau protein or fragment thereof may be detected using a method comprising mass spectrometry, for example immunoprecipitation followed by mass spectrometry. The mass spectrometry may be LC-MS.

### First and Second Specific Binding Molecules.

The method may comprise contacting the sample with a first specific binding molecule that binds to an epitope within residues 296 to 391, preferably within residues 307 to 391, of SEQ ID NO: 1 and contacting the sample with a second specific binding molecule that binds to an epitope within SEQ ID NO: 1, preferably within residues 151 to 243 of SEQ ID NO: 1. Any and all combinations of a first specific binding molecule disclosed herein that binds to an epitope within residues 296 to 391, preferably within residues 307 to 391, of SEQ ID NO: 1 and contacting the sample with a second specific binding molecule disclosed herein that binds to an epitope within SEQ ID NO: 1, preferably within residues 151 to 243 of SEQ ID NO: 1, are explicitly contemplated herein.

The first specific binding molecule may bind to SEQ ID NO: 1 or a fragment thereof with a K_{D} of less than 25 nM, less than 20 nM, less than 15 nM, less than 10 nM, less than 8 nM, less than 6 nM, less than 5 nM, less than 4 nM, less than 3 nM, less than 2 nM, less than 1 nM, less than 0.5 nM, less than 0.4 nM, less than 0.3 nM, less than 0.2 nM or less than 0.15 nM. A high affinity first specific binding molecule may be especially advantageous in embodiments where the first specific binding molecule is a surface bound specific binding molecule. A preferred high affinity first specific binding molecule is a specific binding molecule comprising the CDRs of S1D12. A preferred high affinity first specific binding molecule is a specific binding molecule comprising the CDRs and FWs of S1D12. A preferred high affinity first specific binding molecule is a specific binding molecule comprising the VH and/or VL domains of S1D12.

The first specific binding molecule may bind to an epitope within residues 296 to 391 of SEQ ID NO: 1. Preferably, the first specific binding molecule binds to an epitope within residues 307 to 391 of SEQ ID NO: 1. Preferably, the first specific binding molecule binds to an epitope within residues 337 to 355 of SEQ ID NO: 1 or within residues 367 to 379 of SEQ ID NO: 1.

The first specific binding molecule may bind to an epitope within residues 367 to 379 of SEQ ID NO: 1. A preferred first specific binding molecule is a specific binding molecule comprising the CDRs of S1G2. A preferred high affinity first specific binding molecule is a specific binding molecule comprising the CDRs and FWs of S1G2. A preferred high affinity first specific binding molecule is a specific binding molecule comprising the VH and/or VL domains of S1G2.

The first specific binding molecule may bind to an epitope within residues 355 to 367 of SEQ ID NO: 1. A preferred first specific binding molecule is a specific binding molecule comprising the CDRs of CA4. A preferred high affinity first specific binding molecule is a specific binding molecule comprising the CDRs and FWs of CA4. A preferred high affinity first specific binding molecule is a specific binding molecule comprising the VH and/or VL domains of CA4.

The second specific binding molecule may bind to the same epitope or to a different epitope than the first specific binding molecule. The skilled person will understand that an appropriate pair of antibodies may permit detection of particular tau fragments of interest. For instance, where the epitope of the first specific binding molecule and the second specific binding molecule are widely spaced across the sequence of SEQ ID NO:1 (such as binding to an N-terminal region and to a C-terminal region respectively), the method may selectively detect full length and longer fragments of tau; shorter fragments of tau that are only bound by one of the specific binding molecules (or by neither) will not be detected.

The second specific binding molecule may be a known specific binding molecule such as HT7, BT2, Tau12 or Tau146. The second specific binding molecule may be HT7 or BT2.

In preferred configurations, the first specific binding molecule may be S1D12, S1G2 or CA4 and the second specific binding molecule may be BT2 or HT7.

In preferred configurations, the first specific binding molecule may be S1D12 and the second specific binding molecule may be BT2 or HT7. The first specific binding molecule may be S1D12 and the second specific binding molecule may be BT2. The first specific binding molecule may be S1D12 and the second specific binding molecule may be HT7.

In preferred configurations, the first specific binding molecule may be S1G2 and the second specific binding molecule may be BT2 or HT7. The first specific binding molecule may be S1G2 and the second specific binding molecule may be BT2. The first specific binding molecule may be S1G2 and the second specific binding molecule may be HT7.

In preferred configurations, the first specific binding molecule may be CA4 and the second specific binding molecule may be BT2 or HT7. The first specific binding molecule may be CA4 and the second specific binding molecule may be BT2. The first specific binding molecule may be CA4 and the second specific binding molecule may be HT7.

In preferred configurations, the first specific binding molecule may be S1D12 or S1G2 and the second specific binding molecule may be BT2 or HT7.

Alternative second specific binding molecules to HT7 and/or BT2 described elsewhere herein may be substituted for HT7 and/or BT2 in any of the preferred configurations identified above. For example, references to HT7 and/or BT2 may be substituted with references to a second specific binding molecule comprising the CDRs (optionally further comprising the FW regions and optionally the VH and/or VL domains) of 3aA6, 3aD6, 3bD11, CB11, CA2, CB6, CA7, CA8, CB10, CC7, CB12, CC3, CA1, CA3, CD2, CC4, CD1 or CC5. References to S1D12, S1G2 or CA4 in the preferred configurations identified above include first specific binding molecules comprising the CDRs (optionally further comprising the FW regions and optionally the VH and/or VL domains) of S1D12, S1G2 or CA4.

The ability to interrogate and determine the levels of various tau species or fragments in patient samples is crucial in early diagnosis of tauopathies. The invention provides a method for determining the concentrations of different tau species accordingly. The method may use spiked samples. The method may use pairings of specific binding molecules directed towards selected epitopes of the tau protein. Accordingly, the method may be a sandwich ELISA assay.

When the first specific binding molecule binds to an epitope within residues 337 to 355 of SEQ ID NO: 1 and the second specific binding molecule binds to an epitope within residues 367 to 379 of SEQ ID NO: 1, the method may detect SEQ ID NO:1, SEQ ID NO: 3, SEQ ID NO: 4, SEQ ID NO: 5, SEQ ID NO: 6 and SEQ ID NO: 7. Since cleavage within the from proteolytically stable core of the paired helical filament (PHF) is thought to be uncommon, this embodiment may therefore detect total tau comprising the PHF.

The second specific binding molecule may bind to an epitope within residues 379 to 391 of SEQ ID NO: 1. A preferred second specific binding molecule is a specific binding molecule comprising the CDRs of E2E8.

As described herein, specific binding molecules that bind to an epitope within residues 379 to 391 of SEQ ID NO: 1 may be “E-specific”; that E391 may be critical for binding. When the first specific binding molecule binds to an epitope within residues 337 to 355 of SEQ ID NO: 1 and the second specific binding molecule binds to an epitope within residues 379 to 391 of SEQ ID NO: 1, the method may detect SEQ ID NO:1, SEQ ID NO: 3 and SEQ ID NO: 4. Since E-specific specific binding molecules do not detect SEQ ID NO: 5, SEQ ID NO: 6 or SEQ ID NO: 7, this embodiment may therefore detect dGAE and full length tau but not fragments lacking E391 such as dGA.

The second specific binding molecule may bind to an epitope within residues 13 to 25 of SEQ ID NO: 1. A preferred second specific binding molecule is a specific binding molecule comprising the CDRs of CB7.

When the first specific binding molecule binds to an epitope within residues 337 to 355 of SEQ ID NO: 1 and the second specific binding molecule binds to an epitope within residues 13 to 25 of SEQ ID NO: 1, the method may detect SEQ ID NO:1. However, this embodiment will not detect isolated SEQ ID NO: 3, SEQ ID NO: 4, SEQ ID NO: 5, SEQ ID NO: 6 and SEQ ID NO: 7 as these fragments omit residues 13 to 25 of SEQ ID NO: 1. Since the epitopes used in this embodiment are spaced widely apart, this embodiment may therefore detect full-length tau.

The method may comprise contacting the sample with at least one pair of first and second specific binding molecules, wherein the pair of first and second specific binding molecules may be any two specific binding molecules of the invention. Preferred pairs of first and second specific binding molecule include:
A first specific binding molecule that binds to an epitope within residues 337 to 355 of SEQ ID NO: 1 and a second specific binding molecule that binds to an epitope within residues 367 to 379 of SEQ ID NO: 1;
A first specific binding molecule that binds to an epitope within residues 337 to 355 of SEQ ID NO: 1 and a second specific binding molecule that binds to an epitope within residues 379 to 391 of SEQ ID NO: 1; and
A first specific binding molecule that binds to an epitope within residues 337 to 355 of SEQ ID NO: 1 and a second specific binding molecule that binds to an epitope within residues 13 to 25 of SEQ ID NO: 1.

The method may comprise contacting the sample with at least two or at least three pairs of first and second specific binding molecules. For instance, the method may comprise contacting the sample with:
A first specific binding molecule that binds to an epitope within residues 337 to 355 of SEQ ID NO: 1 and a second specific binding molecule that binds to an epitope within residues 367 to 379 of SEQ ID NO: 1;
A first specific binding molecule that binds to an epitope within residues 337 to 355 of SEQ ID NO: 1 and a second specific binding molecule that binds to an epitope within residues 379 to 391 of SEQ ID NO: 1; and
A first specific binding molecule that binds to an epitope within residues 337 to 355 of SEQ ID NO: 1 and a second specific binding molecule that binds to an epitope within residues 13 to 25 of SEQ ID NO: 1.

Where the method comprises contacting the sample with two or more pairs of specific binding molecules, each pair of specific binding molecules is typically contacted to the sample separately and/or in parallel. The sample may therefore be aliquoted prior to contacting the sample with the pairs of specific binding molecules. A separate aliquot may be contacted with each pair of specific binding molecules. Contacting in parallel may be contacting at the same time or simultaneously. Contacting in parallel may be contacting at essentially the same time or essentially simultaneously. Contacting in parallel may be not contacting sequentially or contacting one after another. Contacting in parallel typically means each pair of specific binding molecules is contacted to the sample in a separate vessel. Contacting in parallel typically means that each pair of specific binding molecules may independently interact with the sample.

Each pair of specific binding molecules may be configured to detect a different tau protein or a fragment thereof. For example:
A first pair of specific binding molecules that bind to an epitope within residues 337 to 355 of SEQ ID NO: 1 and to an epitope within residues 367 to 379 of SEQ ID NO: 1, respectively, may be configured to detect total tau comprising the PHF;
A second pair of specific binding molecules that bind to an epitope within residues 337 to 355 of SEQ ID NO: 1 and to an epitope within residues 379 to 391 of SEQ ID NO: 1, respectively, may be configured to detect dGAE and full-length tau but not fragments lacking E391 such as dGA; and
A third pair of specific binding molecules that bind to an epitope within residues 337 to 355 of SEQ ID NO: 1 and to an epitope within residues 13 to 25 of SEQ ID NO: 1, respectively, may be configured to detect full-length tau.

The method may further comprise a step of determining the levels of the different tau proteins or a fragment(s) thereof. The method may further comprise a step of comparing the levels of the different tau proteins or a fragment(s) thereof.

In a specific embodiment, the method comprises detecting a tau protein or a fragment thereof in a sample comprising:
a) contacting the sample with a first pair of specific binding molecules comprising a specific binding molecule comprising the CDRs of S1D12 and a specific binding molecule comprising the CDRs of S1G2;
b) contacting the sample with a second pair of specific binding molecules comprising a specific binding molecule comprising the CDRs of S1D12 and a specific binding molecule comprising the CDRs of E2E8; and
c) contacting the sample with a third pair of specific binding molecules comprising a specific binding molecule comprising the CDRs of S1D12 and a specific binding molecule comprising the CDRs of CB7;
wherein each pair of specific binding molecules is contacted to the sample in parallel.

The invention provides distinguishing and/or determining the levels of hT40, dGAE and dGA in a sample accordingly.

### Tau Fragments

The method may be an in vitro method for detecting a tau protein fragment. The amino acid sequence of the fragment may consist of amino acid residues within residues 113 to 379 of SEQ ID NO: 1. The present inventors have identified tau fragments which can be detected by core binding specific binding molecules in plasma. The inventors have identified that the fragment may omit certain N-terminal and C-terminal amino acid residues, specifically the fragments do not include residues 1 to 112 or residues 380 to 441 of SEQ ID NO: 1.

The fragment may comprise at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, or at least 175 consecutive amino acid residues from within residues 113 to 379 of SEQ ID NO: 1.

As used herein, the term “consecutive” may be substituted for the term “contiguous”, each referring to amino acid residues found adjacent to one another in a primary amino acid sequence. Accordingly, the fragment may consist of consecutive or contiguous amino acid residues within residues 113 to 379 of SEQ ID NO: 1.

The fragment may comprise at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 261, at least 262, at least 263, at least 264, at least 265, at least 266 or 267 consecutive amino acid residues from within residues 113 to 379 of SEQ ID NO: 1.

The fragment may comprise at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 261, at least 262, at least 263, at least 264, at least 265, at least 266 or 267 consecutive amino acid residues from within residues 113 to 379 of SEQ ID NO: 1.

The fragment may be defined by any one or more of the number of consecutive amino acids from within residues 113 to 379 of SEQ ID NO: 1, the number of consecutive amino acid residues from residues 297 to 391 of SEQ ID NO: 1; the number of consecutive amino acid residues from residues 244 to 296 of SEQ ID NO: 1; the number of consecutive amino acid residues from residues 151 to 243 of SEQ ID NO: 1 and/or the number of consecutive amino acid residues from residues 113 to 150 of SEQ ID NO: 1. In any embodiment comprising amino acid residues from 151 to 243 and from 297 to 391 of SEQ ID NO: 1, residues 244 to 296 of SEQ ID NO: 1 may additionally be present and will typically be present in between the residues from 151 to 243 and from 297 to 391 of SEQ ID NO: 1 such that the fragment comprises consecutive amino acid residues from residues 151 to 243, residues 244 to 296 and residues 297 to 391 of SEQ ID NO: 1 which are consecutive amino acid residues from within residues 113 to 379 of SEQ ID NO: 1.

The fragment may comprise at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23 at least 24, at least 25, at least 26, at least 27, at least 28, at least 29 or at least 30 consecutive amino acid residues from residues 297 to 391 of SEQ ID NO: 1. Residues 297 to 391 of SEQ ID NO: 1 (see also SEQ ID NO: 4) correspond to the predominant fragment isolated from proteolytically stable core of the paired helical filament (PHF).

The fragment may comprise at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23 at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 91, at least 92, at least 93, at least 94, or 95 consecutive amino acid residues from residues 297 to 391 of SEQ ID NO: 1.

Wherein the fragment comprises at least 4 consecutive amino acid residues from residues 297 to 391 of SEQ ID NO: 1, the at least 4 consecutive amino acid residues may comprise amino acid residues 337 to 349 of SEQ ID NO: 1 and/or amino acid residues 370 to 374 of SEQ ID NO: 1. The fragment may comprise the epitope of S1D12 and/or S1G2.

The fragment may comprise at least 30, at least 35, at least 40, at least 45, at least 50, at least 55, at least 60, at least 65, at least 70, at least 75, at least 80, at least 85, at least 90, at least 91, at least 92, at least 93, at least 94, or 95 consecutive amino acid residues from residues 297 to 391 of SEQ ID NO: 1.

The fragment may comprise 38 consecutive amino acid residues from residues 297 to 391 of SEQ ID NO: 1. The 38 consecutive amino acids may be from residues 337 to 349 of SEQ ID NO: 1. The fragment may comprise the epitopes of S1D12 and S1G2. The fragment may comprise the epitopes of S1D12 and S1G2 and the intervening consecutive amino acid residues.

The fragment may comprise at least 40, at least 45, at least 50, at least 55, at least 60, at least 65, at least 70, at least 75, at least 80, at least 85, at least 90, at least 91, at least 92, at least 93, at least 94, or 95 consecutive amino acid residues from residues 297 to 391 of SEQ ID NO: 1.

The fragment may comprise at least 50, at least 55, at least 60, at least 65, at least 70, at least 75, at least 80, at least 85, at least 90, at least 91, at least 92, at least 93, at least 94, or 95 consecutive amino acid residues from residues 297 to 391 of SEQ ID NO: 1.

The fragment may comprise 53 consecutive amino acid residues from residues 297 to 391 of SEQ ID NO: 1. The 53 consecutive amino acids may be from residues 297 to 349 of SEQ ID NO: 1. The fragment may comprise consecutive amino acid residues from residue 297 to the C-terminal end of the epitope of S1D12.

The fragment may comprise 78 consecutive amino acid residues from residues 297 to 391 of SEQ ID NO: 1. The 78 consecutive amino acids may be from residues 297 to 374 of SEQ ID NO: 1. The fragment may comprise consecutive amino acid residues from residue 297 to the C-terminal end of the epitope of S1G2.

The fragment may comprise at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23 at least 24, at least 25, at least 26, at least 27, at least 28, at least 29 or at least 30 consecutive amino acid residues from residues 151 to 243 of SEQ ID NO: 1. Two proline rich domains span between amino acid residues from residues 151 to 243 of SEQ ID NO: 1. The fragment may comprise at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23 at least 24, at least 25, at least 26, at least 27, at least 28, at least 29 or at least 30 consecutive amino acid residues from at least one proline rich domain.

Wherein the fragment comprises at least 4 consecutive amino acid residues from residues 151 to 243 of SEQ ID NO: 1, the at least 4 consecutive amino acid residues may comprise amino acid residues 194 to 198 of SEQ ID NO: 1 and/or amino acid residues 159 to 163 of SEQ ID NO: 1. The fragment may comprise the epitope of BT2 and/or HT7.

The fragment may comprise 48 consecutive amino acid residues from residues 151 to 243 of SEQ ID NO: 1. The 48 consecutive amino acids may be from residues 159 to 198 of SEQ ID NO: 1. The fragment may comprise the epitopes of BT2 and HT7. The fragment may comprise the epitopes of BT2 and HT7 and the intervening consecutive amino acid residues.

The fragment may comprise at least 50, at least 55, at least 60, at least 65, at least 70, at least 75, at least 80, at least 85, at least 90, at least 91, at least 92, at least 93, at least 94, or 95 consecutive amino acid residues from residues 159 to 198 of SEQ ID NO: 1.

The fragment may comprise at least 60, at least 65, at least 70, at least 75, at least 80, at least 85, at least 90, at least 91, at least 92, at least 93, at least 94, or 95 consecutive amino acid residues from residues 159 to 198 of SEQ ID NO: 1.

The fragment may comprise 50 consecutive amino acid residues from residues 151 to 243 of SEQ ID NO: 1. The 50 consecutive amino acids may be from residues 194 to 243 of SEQ ID NO: 1. The fragment may comprise consecutive amino acid residues from the N-terminal start of the epitope of BT2 to residue 243.

The fragment may comprise 85 consecutive amino acid residues from residues 151 to 243 of SEQ ID NO: 1. The 85 consecutive amino acids may be from residues 159 to 243 of SEQ ID NO: 1. The fragment may comprise consecutive amino acid residues from the N-terminal start of the epitope of HT7 to residue 243.

The fragment may comprise at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23 at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 91, at least 92, or 93 consecutive amino acid residues from residues 151 to 243 of SEQ ID NO: 1. Two proline rich domains span between amino acid residues from residues 151 to 243 of SEQ ID NO: 1. The fragment may comprise at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23 at least 24, at least 25, at least 26, at least 27, at least 28, at least 29, at least 30, at least 35, at least 40, at least 45, at least 50, at least 55, at least 60, at least 65, at least 70, at least 75, at least 80, at least 85, at least 90, at least 91, at least 92, or 93 consecutive amino acid residues from at least one proline rich domain.

The fragment may comprise at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23 at least 24, at least 25, at least 26, at least 27, at least 28, at least 29, at least 30, at least 35, at least 40, at least 45, at least 50, at least 51, at least 52, at least 53 or 54 consecutive amino acid residues from residues 244 to 296 of SEQ ID NO: 1. Typically the consecutive amino acid residues from residues 244 to 296 of SEQ ID NO: 1 will include residue 244 and/or residue 296 of SEQ ID NO: 1. The fragment may comprise all 54 consecutive amino acid residues from residues 244 to 296 of SEQ ID NO: 1.

The fragment may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23 at least 24, at least 25, at least 26, at least 27, at least 28, at least 29, at least 30, at least 31, at least 32, at least 33, at least 34, at least 35, at least 36, at least 37 or 38 consecutive amino acid residues from residues 113 to 150 of SEQ ID NO: 1. Typically the consecutive amino acid residues from residues 113 to 150 of SEQ ID NO: 1 will include residue 150 of SEQ ID NO: 1. The fragment may comprise all 38 consecutive amino acid residues from residues 113 to 150 of SEQ ID NO: 1.

The fragment may comprise at least 30 consecutive amino acid residues from within residues 113 to 379 of SEQ ID NO: 1 and
(a) at least 4 consecutive amino acid residues from residues 297 to 391 of SEQ ID NO: 1; and/or
(b) at least 4 consecutive amino acid residues from residues 151 to 243 of SEQ ID NO: 1.

The fragment may comprise at least 156 consecutive amino acid residues from within residues 113 to 379 of SEQ ID NO: 1 and
(a) at least 4 consecutive amino acid residues from residues 297 to 391 of SEQ ID NO: 1, optionally comprising residues 337 to 349 of SEQ ID NO: 1; and/or
(b) at least 4 consecutive amino acid residues from residues 151 to 243 of SEQ ID NO: 1, optionally comprising residues 194 to 198 of SEQ ID NO: 1.

The fragment may comprise at least 181 consecutive amino acid residues from within residues 113 to 379 of SEQ ID NO: 1 and
(a) at least 4 consecutive amino acid residues from residues 297 to 391 of SEQ ID NO: 1, optionally comprising residues 370 to 374 of SEQ ID NO: 1; and/or
(b) at least 4 consecutive amino acid residues from residues 151 to 243 of SEQ ID NO: 1, optionally comprising residues 194 to 198 of SEQ ID NO: 1.

The fragment may comprise at least 191 consecutive amino acid residues from within residues 113 to 379 of SEQ ID NO: 1 and
(a) at least 4 consecutive amino acid residues from residues 297 to 391 of SEQ ID NO: 1, optionally comprising residues 337 to 349 of SEQ ID NO: 1; and/or
(b) at least 4 consecutive amino acid residues from residues 151 to 243 of SEQ ID NO: 1, optionally comprising residues 159 to 163 of SEQ ID NO: 1.

The fragment may comprise at least 216 consecutive amino acid residues from within residues 113 to 379 of SEQ ID NO: 1 and
(a) at least 4 consecutive amino acid residues from residues 297 to 391 of SEQ ID NO: 1, optionally comprising residues 370 to 374 of SEQ ID NO: 1; and/or
(b) at least 4 consecutive amino acid residues from residues 151 to 243 of SEQ ID NO: 1, optionally comprising residues 159 to 163 of SEQ ID NO: 1.

The fragment may comprise at least 156 consecutive amino acid residues from within residues 113 to 379 of SEQ ID NO: 1 and
(a) at least 53 consecutive amino acid residues from residues 297 to 391 of SEQ ID NO: 1, optionally comprising residues 337 to 349 of SEQ ID NO: 1; and/or
(b) at least 50 consecutive amino acid residues from residues 151 to 243 of SEQ ID NO: 1, optionally comprising residues 194 to 198 of SEQ ID NO: 1.

The fragment may comprise at least 181 consecutive amino acid residues from within residues 113 to 379 of SEQ ID NO: 1 and
(a) at least 78 consecutive amino acid residues from residues 297 to 391 of SEQ ID NO: 1, optionally comprising residues 370 to 374 of SEQ ID NO: 1; and/or
(b) at least 50 consecutive amino acid residues from residues 151 to 243 of SEQ ID NO: 1, optionally comprising residues 194 to 198 of SEQ ID NO: 1.

The fragment may comprise at least 191 consecutive amino acid residues from within residues 113 to 379 of SEQ ID NO: 1 and
(a) at least 53 consecutive amino acid residues from residues 297 to 391 of SEQ ID NO: 1, optionally comprising residues 337 to 349 of SEQ ID NO: 1; and/or
(b) at least 85 consecutive amino acid residues from residues 151 to 243 of SEQ ID NO: 1, optionally comprising residues 159 to 163 of SEQ ID NO: 1.

The fragment may comprise at least 216 consecutive amino acid residues from within residues 113 to 379 of SEQ ID NO: 1 and
(a) at least 78 consecutive amino acid residues from residues 297 to 391 of SEQ ID NO: 1, optionally comprising residues 370 to 374 of SEQ ID NO: 1; and/or
(b) at least 85 consecutive amino acid residues from residues 151 to 243 of SEQ ID NO: 1, optionally comprising residues 159 to 163 of SEQ ID NO: 1.

The fragment may comprise at least 30 consecutive amino acid residues from residues 297 to 391 of SEQ ID NO: 1 and/or at least 30 consecutive amino acid residues from residues 151 to 243 of SEQ ID NO: 1.

The fragment may comprise at least 50 consecutive amino acid residues from residues 297 to 391 of SEQ ID NO: 1 and/or at least 50 consecutive amino acid residues from residues 151 to 243 of SEQ ID NO: 1.

The fragment may comprise at least 75 consecutive amino acid residues from residues 297 to 391 of SEQ ID NO: 1 and/or at least 75 consecutive amino acid residues from residues 151 to 243 of SEQ ID NO: 1.

The fragment may comprise at least 38 consecutive amino acid residues from residues 297 to 391 of SEQ ID NO: 1 and/or at least 48 consecutive amino acid residues from residues 151 to 243 of SEQ ID NO: 1.

The fragment may comprise at least 53 consecutive amino acid residues from residues 297 to 391 of SEQ ID NO: 1 and/or at least 50 consecutive amino acid residues from residues 151 to 243 of SEQ ID NO: 1.

The fragment may comprise at least 78 consecutive amino acid residues from residues 297 to 391 of SEQ ID NO: 1 and/or at least 85 consecutive amino acid residues from residues 151 to 243 of SEQ ID NO: 1.

The fragment may comprise amino acid residues 337 to 349 of SEQ ID NO: 1 and/or amino acid residues 370 to 374 of SEQ ID NO: 1.

The fragment may comprise amino acid residues 194 to 198 of SEQ ID NO: 1 and/or amino acid residues 159 to 163 of SEQ ID NO: 1.

The fragment may comprise at least 140 consecutive amino acid residues from within residues 113 to 379 of SEQ ID NO: 1 and
(a) at least 50 consecutive amino acid residues from residues 297 to 391 of SEQ ID NO: 1; and/or
(b) at least 50 consecutive amino acid residues from residues 151 to 243 of SEQ ID NO: 1.

The fragment may comprise at least 160 consecutive amino acid residues from within residues 113 to 379 of SEQ ID NO: 1 and
(a) at least 60 consecutive amino acid residues from residues 297 to 391 of SEQ ID NO: 1; and/or
(b) at least 60 consecutive amino acid residues from residues 151 to 243 of SEQ ID NO: 1.

The N-terminal residue of the fragment may be a residue selected from the group consisting of all residues from the 113^{th} residue of SEQ ID NO: 1 to the 193^{rd} residue of SEQ ID NO: 1.

The N-terminal residue of the fragment may be a residue selected from the group consisting of all residues from the 113^{th} residue of SEQ ID NO: 1 to the 159^{th} residue of SEQ ID NO: 1.

The N-terminal residue of the fragment may be a residue selected from the group consisting of all residues from the 159^{th} residue of SEQ ID NO: 1 to the 193^{rd} residue of SEQ ID NO: 1.

The N-terminal residue of the fragment may be a residue selected from the group consisting of all residues from the 163^{rd} residue of SEQ ID NO: 1 to the 193^{rd} residue of SEQ ID NO: 1.

The N-terminal residue of the fragment may be residue 113 of SEQ ID NO: 1

The C-terminal residue of the fragment may be a residue selected from the group consisting of all residues from the 350^{th} residue of SEQ ID NO: 1 to the 379^{th} residue of SEQ ID NO: 1.

The C-terminal residue of the fragment may be a residue selected from the group consisting of all residues from the 350^{th} residue of SEQ ID NO: 1 to the 370^{th} residue of SEQ ID NO: 1.

The C-terminal residue of the fragment may be a residue selected from the group consisting of all residues from the 350^{th} residue of SEQ ID NO: 1 to the 374^{th} residue of SEQ ID NO: 1.

The fragment consisting of amino acid residues within residues 113 to 379 of SEQ ID NO: 1 may comprise amino acid residues:
(a) 194 to 349 of SEQ ID NO: 1;
(b) 159 to 349 of SEQ ID NO: 1;
(c) 194 to 374 of SEQ ID NO: 1;
(d) 159 to 374 of SEQ ID NO: 1;
(e) 195 to 370 of SEQ ID NO: 1;
(f) 159 to 379 of SEQ ID NO: 1;
(g) 113 to 224 of SEQ ID NO: 1;
(h) 113 to 349 of SEQ ID NO: 1; or
(i) 113 to 370 of SEQ ID NO: 1.

Taken together, the data disclosed herein indicate that in human plasma using S1D12 as a first specific binding molecule and BT2 as a second specific binding molecule detects a fragment consisting of amino acid residues within residues 113 to 379 of SEQ ID NO: 1 comprising amino acid residues 194 to 349 of SEQ ID NO: 1.

Taken together, the data disclosed herein indicate that in human plasma using S1D12 as a first specific binding molecule and HT7 as a second specific binding molecule detects a fragment consisting of amino acid residues within residues 113 to 379 of SEQ ID NO: 1 comprising amino acid residues 159 to 349 of SEQ ID NO: 1.

Taken together, the data disclosed herein indicate that in human plasma using S1G2 as a first specific binding molecule and BT2 as a second specific binding molecule detects a fragment consisting of amino acid residues within residues 113 to 379 of SEQ ID NO: 1 comprising amino acid 194 to 374 of SEQ ID NO: 1.

Taken together, the data disclosed herein indicate that in human plasma using S1G2 as a first specific binding molecule and HT7 as a second specific binding molecule detects a fragment consisting of amino acid residues within residues 113 to 379 of SEQ ID NO: 1 comprising amino acid 159 to 374 of SEQ ID NO: 1.

The inventors have performed multiple Simoa® assays to detect human plasma tau levels, together with mass spectrometry, which together suggest that the release of “extended core” fragments of tau (within a consensus plasma tau sequence residues 113-379 of SEQ ID NO: 1) into plasma appears to be part of normal processing of tau protein. Such fragments have been shown by the inventors to be significantly reduced in Alzheimer's Disease plasma samples compared to cognitively unimpaired controls. The folding of extended core fragments may occlude core epitopes leading to advantages for detection of core fragments for high affinity specific binding molecules described in PCT application no. PCT/EP2021/069160, such as S1D12 and S1G2.

The amino acid sequence of the fragment may consist of amino acid residues within residues:
(a) 113 to 370 of SEQ ID NO: 1;
(b) 113 to 349 of SEQ ID NO: 1;
(c) 113 to 224 of SEQ ID NO: 1;
(d) 159 to 379 of SEQ ID NO: 1;
(e) 195 to 370 of SEQ ID NO: 1;
(f) 194 to 349 of SEQ ID NO: 1;
(g) 159 to 349 of SEQ ID NO: 1;
(h) 194 to 374 of SEQ ID NO: 1; or
(i) 159 to 374 of SEQ ID NO: 1;

The fragment may consist of amino acid residues 113 to 379 of SEQ ID NO: 1.

In an alternative statement of the first aspect of the invention, for any fragment defined herein with reference to one or more amino acid residues of SEQ ID NO: 1, one or more amino acid residues may be alternatively stated as “about” the same amino acid residue. For instance, an alternative statement of the phrase “The fragment may consist of amino acid residues 113 to 379 of SEQ ID NO: 1” is “The fragment may consist of amino acid residues about 113 to about 379 of SEQ ID NO: 1”. The term “about” in this context means plus or minus 10, or more preferably, plus or minus 9, plus or minus 8, plus or minus 7, plus or minus 6, plus or minus 5, plus or minus 4, plus or minus 3, plus or minus 2, or plus or minus 1. Instead of alternatively stating a residue number using the word “about”, a range of residue numbers centred on the residue number may be stated. For example, an alternative statement of the phrase “The fragment may consist of amino acid residues 113 to 379 of SEQ ID NO: 1” is “The fragment may consist of amino acid residues from 103-123 to 369-389 of SEQ ID NO: 1”.

The sequence of tau may vary between individuals due to differences in their genetic code and/or somatic mutation. Any fragment defined herein may be a sequence variant of the amino acid residues within residues 113 to 379 of SEQ ID NO: 1. Such sequence variants may be described as variant fragments accordingly and the fragment with amino acid residues corresponding to those within residues 113 to 379 of SEQ ID NO: 1 may be described as the wild type or canonical fragment. A variant fragment may for instance, have at least 95%, at least 96%, at least 97%, at least 98% or at least 99% identity to the wild type or canonical fragment. A variant fragment may for instance, have at least one, at least two, at least three, at least four, at least five, at least six, at least seven, at least eight, at least nine or at least ten amino acid substitutions relative to the wild type or canonical fragment. The amino acid substitutions may be conservative amino acid substitutions.

The method may further comprise confirming the identity of the fragment. Confirming the identity of the fragment may for instance be by analysing the fragment by mass spectrometry. The mass spectrometry may be liquid chromatography-mass spectrometry analysis (LC-MS). Confirming the identity of the fragment may alternatively be by assaying for one or more further tau fragments, wherein the fragment whose identify is to be confirmed may be referred to as the first fragment. Assaying for one or more further tau fragments may comprise assaying for a shorter tau fragment and/or assaying for a longer tau fragment. Appropriate such assays will be apparent to the skilled person based on the content of this disclosure and depending on the length of the first fragment. For example, using a more N-terminally targeted antibody (such as Tau12 which binds to residues 6-18 of SEQ ID NO: 1) or a more C-terminally targeted antibody (such as Tau46 which binds to residues 404-441). Assaying for one or more further tau fragments may further comprise comparing the levels of the shorter tau fragment and/or the longer tau fragment with the levels of the first fragment. Where substantially no shorter tau fragment and/or longer tau fragment is detected, the identity of the first tau fragment may be confirmed. Where the shorter tau fragment and/or longer tau fragment is below the lower limit of quantification, the identity of the first tau fragment may be confirmed.

The method may comprise detecting two or more fragments of tau disclosed herein.

The method may further comprise determining a concentration of the tau protein fragment in the sample.

### Diagnostics

According to a further aspect, the invention provides a diagnostic method comprising contacting a saliva sample with a specific binding molecule wherein the first specific binding molecule binds to an epitope within residues 297 to 391 of SEQ ID NO: 1.

The diagnostic method may comprise the in vitro method for detecting a tau protein or a fragment thereof in a sample according to the first aspect.

The method may further comprise diagnosing a tauopathy if a tau protein or a fragment thereof is detected.

The method may further comprise treating a tauopathy if a tau protein or a fragment thereof is detected and/or if a tauopathy is diagnosed.

The method may further comprise determining a concentration of the tau protein or a fragment thereof in the sample, and optionally comparing the concentration of the tau protein or a fragment thereof in the sample to a concentration of the tau protein or a fragment thereof in a sample from a healthy control or to a predetermined concentration of the tau protein or a fragment thereof indicative of a healthy subject.

### Further Advantages and Embodiments

An advantage of the present invention is the provision of an assay for detecting a tau protein or fragment thereof which can be performed using a saliva sample. Saliva samples are easier, faster and safer to obtain than plasma or CSF samples. However, very limited published data are available supporting a diagnostic test for a tauopathy, particularly Alzheimer's disease, in a saliva sample. The complexities of extracellular tau and the limited progress towards developing a blood-based screen for Alzheimer's disease are reviewed in Chen et al (2019) Alzheimers Dement. 15 (3): 487-496. Chen et al concluded that “most plasma tau is full length”. None of the antibodies described targeted residues 297 to 391 of full-length Tau (see FIG. 1A). The authors suggest using N-terminal assays in a diagnostic context. Two N-terminal assays are described:
NT1 requires a minimal sequence of residues 6 to 198 of full-length tau.
NT2 requires a longer sequence of residues 6 to 224 of full-length tau.

Chen et al suggest use of the NT1 assay, rather than the NT2 assay, in a diagnostic context. Chen et al therefore teach away from using a specific binding molecule that binds to an epitope within residues 296 to 391 of SEQ ID NO: 1, at least in the context of blood-based screening.

Moreover, in plasma samples, core-proline assays performed by the present inventors are more sensitive than the NT1 assay. The NT1 assay is reported as detecting around 2.13 pg/ml plasma tau in healthy controls and around 5.12 pg/ml plasma tau in AD patients (Chen et al (2018)), although both are detected at less than 1 pg/ml in the hands of the present inventors. In contrast, using S1D12 as capture and HT7 as detector detects around 4,200 pg/ml plasma tau in healthy controls and around 520 μg/ml plasma tau in AD patients. Moreover, using S1D12 as capture and BT2 as detector (BT2 is also used in the NT1 assay) detects around 12,800 pg/ml plasma tau in healthy controls and around 1,030 pg/ml plasma tau in AD patients. Without being bound by theory, the release of “extended core” fragments into plasma appears to be part of normal processing of tau protein, leading to higher levels of core—proline fragments than of N-terminal fragments. Such processing of tau protein may be disrupted in AD patients, so that the assays for core—proline fragments of tau in plasma samples disclosed herein represent a surprisingly advantageous diagnostic tool for AD.

The levels of tau measured when using our core capture antibody S1D12 as part of an antibody pair is 1,000-fold more than the typical values seen for the existing NT1 assay in human plasma. In contrast to the reported NT1 assay, where AD/MCI patients typically show higher assay values than healthy controls, for assays using S1D12 capture this pattern is surprisingly reversed with health control samples showing higher tau fragment values than AD/MCI patients. The levels of tau detected using the core capture antibody are significantly greater than previously reported and suggests that the use of S1D12 reveals large amounts of previously undetected tau fragments in biological samples. The use of a specific binding molecule comprising the CDRs of S1D12 therefore provides a surprisingly sensitive AD/MCI diagnostic assay with improved performance over the existing NT1 assay in human plasma samples. These findings translate to higher levels of core-proline fragments in healthy control and form the basis of a regular monitoring test to identify patients worthy of additional screening, i.e. a potential predictor of early onset of disease.

H. Pekeles et al. Alzheimer's & Dementia: Diagnosis, Assessment & Disease Monitoring 11 (2019) 53-6054 describe development and validation of a salivary tau biomarker in Alzheimer's disease. The tau-4 antibody was used to measure t-tau, and all t-tau measures reported in this article used this antibody. Phosphospecific antibodies targeting Thr181, Ser396, and Ser404 and a combination Ser400/Thr403/Ser404 site antibody were used for the analysis of phosphorylation levels at tau sites T181, S396, and S404 and the combination S400, T403, and T404 site, respectively. None of the antibodies described targeted residues 297 to 391 of full-length Tau. The authors therefore make no suggestion to use a first specific binding molecule binds to an epitope within residues 297 to 391 of SEQ ID NO: 1 and instead suggest using a p-tau/t-tau ratio for the S396 phosphorylation site.

The method may comprise contacting the sample with a pair of specific binding molecules that bind to an epitope within residues 337 to 355 of SEQ ID NO: 1 and to an epitope within residues 367 to 379 of SEQ ID NO: 1, respectively.

Preferably, the method comprises contacting the sample with a pair of specific binding molecules comprising a first specific binding molecule comprising the CDRs of S1D12 and a second specific binding molecule comprising the CDRs of S1G2.

A second specific binding molecule used in the method of the invention may be a specific binding molecule comprising the CDRs of CB7 and/or CC7. The epitopes of CB7 and CC7 fall in regions of tau with no homology between the human and mouse tau sequences. The diagnostic utility of these specific binding molecules is demonstrated by their ability to recognise the presence of pathologic human tau in a transgenic mouse brain against a background of endogenous mouse tau protein. These specific binding molecules may therefore be utilised to track the fragmentation patterns of pathologic tau species during aging and in relation to any pharmacologic treatments that may affect the (human) tau protein, its aggregation, movement between compartments in the body (e.g. between brain and blood) and its pattern of fragmentation.

The in vitro method of the invention may comprise contacting the sample with a pair of first and second specific binding molecules, wherein the pair of first and second specific binding molecules wherein the first specific binding molecule binds within the core region of tau (such as to an epitope within residues 337 to 355 or 367 to 379 of SEQ ID NO: 1) and the second specific binding molecule binds to an epitope within residues 13 to 25 of SEQ ID NO: 1. The first specific binding molecule may comprise the CDRs of S1D12 or S1G2. The second specific binding molecule may comprise the CDRs of CB7. Such pairings have the advantage of detecting tau fragments that contain the amino acids spanning from 13-379 of SEQ ID NO:1. The inventors have shown that such fragments decrease with aging in plasma from L66^{+/+} mice, suggesting that either a truncation or epitope occlusion event is occurring or that multiple events of this nature are occurring during the aging of L66^{+/+} mice. Methods enabling detection of tau fragments that contain the amino acids spanning from 13-379 of SEQ ID NO:1 may therefore provide an early marker for pathology associated events.

### Diagnostic Device

According to a second aspect, the invention provides a diagnostic device for use in a method according to the first aspect of the invention.

The device may comprise any suitable components described herein. The device may comprise equipment for measuring the level of a tau protein or a fragment thereof in a sample comprising a sample collection device and an immunoassay. The equipment may further comprise a detector for detecting labelled tau protein or a fragment thereof or labelled antibodies to the tau protein or a fragment thereof in the immunoassay.

The immunoassay or equipment may be incorporated into a miniaturised device for measuring the level of a tau protein or a fragment thereof in a biological sample. Suitably, the device may comprise a lab-on-a-chip.

The device may comprise one or more parts defining an internal channel having an inlet port and a reaction zone.

In some embodiments, the device may comprise a plurality of channels, each with its own inlet port, for measuring the levels of a plurality of different tau protein or a fragment(s) thereof in the sample in parallel. Therefore, each channel may include a different respective immobilised primary antibody or tau protein or a fragment thereof.

Suitably, the device may comprise one or more selectively operable valves associated with the one or more inlet ports for controlling the admission of a sequence of different reagents into to the channels such, for example, as the sample, wash solutions, primary antibody, secondary antibody and enzyme substrate.

The device therefore may comprise a microfluidics device. The channel may include a reaction zone. Microfluidics devices are known to those skilled in the art. A review of microfluidic immunoassays or protein diagnostic chip microarrays is provided by Chin et al. 2012. Lab on a Chip. 2012; 12:2118-2134.

A microfluidics device suitable for carrying out an ELISA immunoassay at a point-of-care is disclosed by Chan C D, Laksanasopin T, Cheung Y K, Steinmiller D et al. “Microfluidics-based diagnostics of infectious diseases in the developing world”. Nature Medicine. 2011; 17(8):1015-1019, the contents of which are incorporated herein by reference.

### Kit

According to a third aspect, the invention provides a kit comprising a specific binding molecule suitable for use in a method according to the first aspect and reagents for detecting a tau protein or fragment thereof in a saliva sample wherein the first specific binding molecule binds to an epitope within residues 297 to 391 of SEQ ID NO: 1.

The kit may comprise a first specific binding molecule that binds to an epitope within residues 297 to 391 of SEQ ID NO: 1 and a second specific binding molecule that binds to an epitope within SEQ ID NO: 1.

The kit may comprise any suitable components described herein. For example, the kit may further comprise reagents suitable for confirming the identity of the fragment.

The kit may comprise an enzyme such, for example, as horseradish peroxidase (HRP). Suitable substrates for HRP are well known in the art and include, for example, ABTS, OPD, AmplexRed, DAB, AEC, TMB, homovanillic acid and luminol.

The kit may comprise one or more specific binding molecules or tau protein or a fragment thereof immobilised on a surface of a well or plate or on the surface of a plurality of magnetic or non-magnetic beads.

The kit may comprise a labelled secondary antibody to the tau protein or a fragment thereof or to a primary antibody to the tau protein or a fragment thereof for quantifying the amount of the tau protein or a fragment thereof bound to surface-bound antibodies or the amount of primary antibody bound to the tau protein or a fragment thereof immobilised on a surface.

Preferred features for the second and subsequent aspects of the invention are as for the first aspect of the invention mutatis mutandis.

The present invention will now be described by way of reference to the following Examples and accompanying Drawings which are present for the purposes of illustration only and are not to be construed as being limiting on the invention.

### Example 1: Sheep Immunisation Using Tau Protein Antigens and Analysis of Antigen Specific Immune Response

Two Welsh bred sheep were hyperimmunised with full length tau protein (2N4R, referred as hT40 in this study) and truncated tau (dGAE, representing the core repeat region corresponding to amino acids 297-391 of hT40) separately to generate antigen specific immune response. For primary immunisation, 500 μg of hT40 or dGAE was mixed with Freund's complete adjuvant in a final volume of up to 2 ml and administered per sheep. For subsequent boosts at 4 weeks intervals, 250 μg of each antigen was mixed with Freund's incomplete adjuvant and administered per sheep. The immune responses were monitored by performing binding ELISA using polyclonal sera collected after 10-14 days of subsequent boosts (FIG. 7).

ELISA plates were coated with 1 μg/ml hT40 or dGAE by incubation at 37° C. for 1 h or 4° C. overnight, followed by blocking with PBS containing 2% Marvel (MPBS) at 37° C. for 1 h. After each step the plates were washed three times with PBS containing 0.1% tween 20 (PBST) and three times with PBS. Sheep polyclonal sera was added to designated wells (pre-immune and subsequent boost samples), double diluted in PBS across the plate and incubated at room temperature for 1 h. Anti-sheep IgG HRP conjugated secondary antibody (Sigma A3415,) was added to the wells and incubated as before. The resulting immunoreaction was developed by adding SureBlue TMB substrate solution, the reaction was stopped using 1 M H2SO4 and the absorbance values measured using a microplate reader at absorbance 450 nm.

Antigen specific immune response was achieved after the first boost for both hT40 and dGAE immunised sheep (FIGS. 7a and 7b) and the levels peaked at boost 2, 3 and 4 without any further improvement in antibody titre. Therefore after 4th boost immunisation, approx. 350 ml of blood was collected from each sheep, PBLs separated using standard techniques and stored in RNA later solution for the extraction of mRNA and further amplification of antibody genes for library construction.

### Example 2: Construction of Phage Display Antibody Libraries from the Immunorepertoire of Sheep

Peripheral blood lymphocytes (PBLs) were prepared from sheep blood using Accuspin system Histopaque 1077 columns (Sigma, Cat No: A7054) according to manufacturer's instructions. Total RNA was extracted using RNeasy midi kit (QIAGEN) and cDNA was synthesised by RT-PCR using sheep antibody constant region-specific primers (OvCHFOR 5′-GAC TTT CGG GGC TGT GGT GGA GGC-3′, OvCKFOR 5′-GA TGG TTT GAA GAG GGA GAC GGA TGG CTG AGC-3′, OvCLFOR 5′-A CAG GGT GAC CGA GGG TGC GGA CTT GG-3′). Sheep IgG VH and VA/VK repertoires were created by PCR amplification as per published methods using V region specific primers (Charlton et al., 2000). For joining of antibody genes, PCR products were enzymatically digested and ligated using the restriction sites AscI for heavy chain and MluI for light chain at the 15 amino acid cellulase linker region incorporated through PCR design. The cloning sites NcoI and NotI were incorporated to the ligated DNA through PCR and the resultant scFv DNA fragments were cloned into a phagemid vector pHEN 2a (Hoogenboom et al 1991). Separate VH-Vλ and VH-Vκ antibody phage display libraries were created by transformation of electrocompetent E. coli TG1 cells (Lucigen Corp).

Two separate phage display libraries were constructed for dGAE and hT40 immunisation hereby referred to as tau antibody library 1 and library 2 respectively. These resultant VH-Vλ and VH-Vκ libraries were rescued separately through helper phage infection following published methods (Charlton et al., 2001) and subjected to biopanning including forced epitope selection for the isolation of phage binders with desired specificity and binding affinity.

### Example 3: Selection and Screening of dGAE and hT40 Phage Display Antibody Libraries

Several biopanning strategies were employed to isolate tau protein specific binders from Library 1 and Library 2.

Helper phage rescued Library 1 was subjected to three selection campaigns as outlined in Table 11. Screening of phage monoclonals using ELISA identified several phage binders, which showed specific binding to the antigens used for selection namely dGA (representing amino acid sequences 297-390 of hT40) and dGAE. These phage binders were grouped into two—(1) dGAE specific binders—(2) dGAE cross reactive binders which recognised dGAE, dGA and hT40. DNA sequencing revealed rich diversity in the selected positive phage population and unique phage clones were reformatted into single chain antibodies (scAbs) by cloning the respective scFv gene (VH-linker-VL) into the bacterial expression vector pIMS147 (reference) using NcoI and NotI restriction enzymes. Unique sequences from these selections were given in a separate document along with sequence IDs.

In order to distinguish between positive clones arising from different selection strategies, the following nomenclature was used.

All positive clones from Library 1 selection 1 were given a prefix ‘E’ (dGAE panning)

All positive clones from Library 1 selection 2 were given a prefix ‘NS’ (non-stringent dGA panning)

All positive clones from Library 1 selection 2 were given a prefix ‘S’ (stringent dGA panning)

All positive clones from Library 1 where a repeat of selection 2 strategy was performed were given a prefix ‘M’

TABLE 12
Showing the three different selection strategies
for Library 1 and the concentrations of dGA or dGAE
antigen used for different rounds of panning.

| | | | |
|---|---|---|---|
| Library 1 | Pan 1 - 50 | Pan 2 - 10 | Pan 2 - 1 |
| Selection 1 | μg/ml dGAE | μg/ml dGAE | μg/ml dGAE |
| Library 1 | Pan 1 - 100 | Pan 2 - 50 | Pan 2 - 10 |
| Selection 2 | μg/ml dGA | μg/ml dGAE | μg/ml dGAE |
| Library 1 | Pan 1 - 100 | Pan 2 - 10 | Pan 2 - 1 |
| Selection 3 | μg/ml dGAE | μg/ml dGAE | μg/ml dGAE |

Similarly, helper phage rescued Library 2 was subjected to five different selection campaigns using the following antigens as outlined in Table 2 and unique phage binders to respective antigens were reformatted into scAbs as described previously. Antigens used for selection are—hT40, R1-3 (representing amino acids in the region 266-359 on hT40) and biotinylated 412-441 (representing amino acids in the region 412-441 on hT40). In selection strategies 4 and 5, a step to deselect dGA binding phage population was introduced to encourage the enrichment of clones outside of 297-390 region on the tau protein.

TABLE 13
Showing the five different selection strategies
for Library 2 and the concentrations of various
antigens used for different rounds of panning.

| | | | |
|---|---|---|---|
| Library 2 | Pan 1 - 100 | Pan 2 - 10 | Pan 2 - 1 |
| Selection 1 | μg/ml hT40 | μg/ml hT40 | μg/ml hT40 |
| Library 2 | Pan 1 - 100 | Pan 2 - 50 | Pan 2 - 10 |
| Selection 2 | μg/ml R1-3 | μg/ml R1-3 | μg/ml R1-3 |
| Library 2 | Pan 1 - 100 | Pan 2 - 10 | Pan 2 - 1 |
| Selection 3 | μg/ml 412-441 | μg/ml 412-441 | μg/ml 412-441 |
| Library 2 | Pan 1 - 100 | Pan 2 - 50 | Pan 2 - 10 |
| Selection 4 | μg/ml hT4Z | μg/ml hT40 dGA | μg/ml hT40 |
| | | deselection | |
| Library 2 | Pan 1 - 100 | Pan 2 - 10 | Pan 2 - 0.1 |
| Selection 5 | μg/ml hT40 | μg/ml hT40 dGA | μg/ml hT40 |
| | | deselection | |

In order to distinguish between positive clones arising from different selection strategies, the following nomenclature was used.

All positive clones from Library 2 selection 1 and selection 2 were given a prefix ‘C’

All positive clones from Library 2 selection 3 were given a prefix ‘412’

All positive clones from Library 2 selection 4 and selection 5 were given prefixes ‘3a’ and ‘3b’ respectively

### Example 4: Expression of Reformatted scAbs in Bacterial System and Purification Using Affinity Chromatography

Bacterial stocks of positive clones were grown in Terrific Broth (TB) medium supplemented with PO4 salts, 100 μg/ml ampicillin and 1% w/v glucose to reach desired cell density, induced with 1 mM IPTG and expressed scAbs in the periplasm was released using the osmotic shock solution (100 ml 200 Mm Tris-HCl-20% sucrose, 200 μl 0.5 M EDTA and 0.5 mg lysozyme followed by 5 Mm MgSO4) and incubating on ice for 15 minutes each. Recombinant anti-tau scAbs present in crude periplasmic extracts were purified using IMAC columns via binding of hexa Histidine tagged protein to activated Ni-sepharose beads and elution using 200 mM Imidazole. Eluted protein samples were dialysed against 1×PBS pH 7.4 and purity analysed on 4-12% Bis-Tris gels using SDS-PAGE. All expressed scAbs were found to be 90% pure. Protein concentrations were determined by running a standard scAb of known concentration alongside unknown samples using SDS-PAGE and comparing the intensities of the protein bands using ImageJ. Alternatively, absorbance values at 280 nm were measured using Ultraspec 6300 pro UV/Visible spectrophotometer (Amersham, Biosciences) and final scAb concentrations determined from the values obtained.

### Example 5: Mapping of Specific Binding Regions and Affinity Ranking of Anti-Tau scAbs

A series of binding ELISA was performed using various truncated versions of the tau protein and 13mer peptide libraries spanning the entire hT40 molecule for mapping the epitopes of anti-tau scAbs (Full list of protein antigens used is given in Table 14). In general, ELISA plates were coated with 1 μg/ml hT40 or dGA or dGAE or other truncated versions of the protein and in the case of biotinylated peptides, plates were coated with 5 μg/ml Streptavidin followed by 1 μg/ml biotinylated peptide. The plates were blocked with 2% MPBS and scAb samples added at desired starting concentrations and double diluted across the plate. Binding was detected using anti-Human C Kappa HRP conjugated secondary antibody and the resulting immunoreaction was developed and absorbance values measured as described above.

For affinity ranking ELISA, plates were coated with 1 μg/ml hT40 or dGA or dGAE as before and blocked as normal. Anti-tau scAbs at a starting concentration of 25 μg/ml or 1 μg/ml was added to designated wells and a serial dilution in 1×PBS was performed for each sample. Binding reaction was determined as before, and the scAbs were ranked based on their absorbance values and top binding scAbs were selected for affinity studies using Surface Plasmon Resonance technology.

TABLE 14
Listing various truncated versions of hT40 and biotinylated
peptide antigens used for epitope mapping of anti-tau scAbs.

| | | | |
|---|---|---|---|
| Tau 1-49 | m186-350 | m268-391 | N biotin 306-323 |
| Tau 1-201 | C2 -186-319 | m352-441 | N biotin 301-325 |
| Tau 1-227 | 42a- 186-350 | m348-441 | N biotin 331-360 |
| B1- 1-391 | m113-319 | Tau 275-305 | N biotin 301-359 |
| Tau 1-251 | m113-201 | Tau 323-335 | N biotin 337-355 |
| Tau 1-319 | m113-251 | Tau 297-315 | C biotin 337-355 |
| Tau 1-155 | m186-251 | dGAE (297-391) | N biotin 339-351 |
| Tau 1-286 | m186-390 + | dGA (297-390) | N biotin 341-353 |
| | D/GA/S | | |
| Tau 1-391 | m186-319/+GAE/ | Tau 337-368 | N biotin 379-390 |
| | DHGAE | | |
| Tau 1-238 | m221-441 | Tau 377-390 | N biotin 390-414 |
| m113-238 | m239-441 | Tau 306-336 | hT40 13mer |
| | | | library N |
| | | | terminal |
| | | | biotinylated |
| m113-155 | m221-319 | Tau 377-391 | |
| m186-441 | m239-319 | Tau 360-378 | |
| m186-391 | m268-441 | N biotin 412-441 | |

### Example 6: Detailed Mapping of Core Region Binding Anti-Tau scAbs

Positive scAbs from Library 1 selections-‘E’, ‘NS’ ‘S’ and ‘M’ clones (as described previously) were checked for hT40, dGA and dGAE binding using direct binding ELISA. scAbs specifically binding to dGAE were grouped as ‘E’ dependant and showed no cross reactivity towards hT40. Selected E specific scAbs and their specific binding to dGAE is shown below (FIG. 8A-E).

TABLE 15
A summary of specific scAb binding to various tau truncations
and protein fragments representing regions of the tau molecule.
A further narrowing down of binding regions was achieved for
some scAbs, including those showing positive reactivity to
shorter epitopes within 13mer peptides is also shown

| | | Shortest |
|---|---|---|
| scAb | Tau proteins/fragments with | region bound |
| clones | positive scAb reactivity | on hT40 |
| E1E8 | 297-391 (dGAE) | 391′E′ |
| E2A6 | 297-391 (dGAE) | 391′E′ |
| E2B7 | 297-391 (dGAE) | 391′E′ |
| E2E8 | 297-391 (dGAE) | 391′E′ |
| E1B8 | 297-390 (dGA), 297-391 | 313-336 |
| | (dGAE), 313-336 | |

Similarly, dGA binding ‘NS’, ‘S’ and ‘M’ group scAbs were subjected to further antigen binding ELISA using shorter tau proteins and biotinylated 13mer peptides as shown below (FIG. 10A-F) and (FIG. 11A-R).

TABLE 16
A summary of specific scAb binding to various tau truncations and protein fragments
representing regions of the tau molecule. A further narrowing down of epitopes
was achieved for some scAbs, including those showing positive reactivity to shorter
epitopes, contained within 13mers, or recognising two epitopes are shown.

| | | Shortest |
|---|---|---|
| scAb | | region bound |
| clones | Tau proteins/fragments with positive scAb reactivity | on hT40 |
| NS3E5 | hT40, 297-390 (dGA), 297-391 (dGAE), 369-390, 369-391 | 369-390 |
| NS3H4 | hT40, 297-390 (dGA), 297-391 (dGAE), 369-390, 369-391 | 369-390 |
| NS4F2 | hT40, 297-390 (dGA), 297-391 (dGAE), 369-390, 369-391 | 369-390 |
| NS4E3 | hT40, 297-390 (dGA), 297-391 (dGAE), 369-390, 369-391 | 369-390 |
| NS3D9 | hT40, 297-390 (dGA), 297-391 (dGAE), 266-359, 337-368 | 337-355 |
| NS1G7 | hT40, 297-390 (dGA), 297-391 (dGAE), 275-305, 337-368 | 275-305, |
| | | 337-368 |
| NS2A3 | hT40, 297-390 (dGA), 297-391 (dGAE), 337-368 | 337-368 |
| NS2A8 | hT40, 297-390 (dGA), 297-391 (dGAE), 337-368 | 337-368 |
| NS2C5 | hT40, 297-390 (dGA), 297-391 (dGAE), 337-368 | 337-368 |
| NS2C8 | hT40, 297-390 (dGA), 297-391 (dGAE), 337-368 | 337-368 |
| NS2D3 | hT40, 297-390 (dGA), 297-391 (dGAE), 337-368 | 337-368 |
| NS2A1 | hT40, 297-390 (dGA), 297-391 (dGAE) | No binding |
| | | to 13mer |
| | | peptides |
| NS2B6 | hT40, 297-390 (dGA), 297-391 (dGAE) | 297-390 |
| NS1B2 | hT40, 297-390 (dGA), 297-391 (dGAE) | 297-390 |
| S1A5 | hT40, 297-390 (dGA), 297-391 (dGAE) | 297-390 |
| S1A12 | hT40, 297-390 (dGA), 297-391 (dGAE) | 297-390 |
| S1E12 | hT40, 297-390 (dGA), 297-391 (dGAE) | 297-390 |
| S1D5 | hT40, 297-390 (dGA), 297-391 (dGAE) | 297-390 |
| S1D12 | hT40, 297-390 (dGA), 297-391 (dGAE), 186-350, 266-359, 337-368 | 337-355 |
| S2C1 | hT40, 297-390 (dGA), 297-391 (dGAE), 186-350, 266-359, 337-368 | 337-355 |
| S1B1 | hT40, 297-390 (dGA), 297-391 (dGAE) | 367-379 |
| S1D2 | hT40, 297-390 (dGA), 297-391 (dGAE), 369-390, 369-391 | 367-379 |
| S1D9 | hT40, 297-390 (dGA), 297-391 (dGAE) | 367-379 |
| S1F4 | hT40, 297-390 (dGA), 297-391 (dGAE), 369-390, 369-391 | 367-379 |
| S1G2 | hT40, 297-390 (dGA), 297-391 (dGAE), 369-390, 369-391 | 367-379 |
| S1G10 | hT40, 297-390 (dGA), 297-391 (dGAE), 369-390, 369-391 | 367-379 |
| S1H6 | hT40, 297-390 (dGA), 297-391 (dGAE), 369-390, 369-391 | 367-379 |
| S1H9 | hT40, 297-390 (dGA), 297-391 (dGAE), 369-390, 369-391 | 367-379 |
| S2C3 | hT40, 297-390 (dGA), 297-391 (dGAE), 369-390, 369-391 | 367-379 |
| S2C6 | hT40, 297-390 (dGA), 297-391 (dGAE), 369-390, 369-391 | 367-379 |
| S2C7 | hT40, 297-390 (dGA), 297-391 (dGAE), 369-390, 369-391 | 367-379 |
| S2D1 | hT40, 297-390 (dGA), 297-391 (dGAE), 369-390, 369-391 | 367-379 |
| S2D4 | hT40, 297-390 (dGA), 297-391 (dGAE), 369-390, 369-391 | 367-379 |
| S2D3 | hT40, 297-390 (dGA), 297-391 (dGAE), 369-390, 369-391, 275-305 | 367-379, |
| | | 275-305 |
| MD9/MoD9 | hT40, 297-390 (dGA), 297-391 (dGAE), 369-390, 369-391 | 373-385 |
| ME12 | hT40, 297-390 (dGA), 297-391 (dGAE), 341-353, 337-349, 339-351 | 337-355 |
| MC5 | hT40, 297-390 (dGA), 297-391 (dGAE), 369-390, 369-391 | 367-379 |
| MD12 | hT40, 297-390 (dGA), 297-391 (dGAE), 369-390, 369-391 | 367-379 |

### Example 7: Detailed Mapping of Binding of ‘C’, ‘412’, ‘3a’ and ‘3b’ Series Clones

‘C’, ‘412’, ‘3a’ and ‘3b’ group scAbs were subjected to further antigen binding ELISA using shorter tau proteins and biotinylated 13mer peptides as shown below (FIG. 12A-F) and (FIG. 13A-R).

TABLE 17
A summary of ‘C’ group scAb binding to various tau truncations and protein fragments representing
regions of the tau molecule. A further narrow down of binding regions achieved for some scAbs, including
those showing positive reactivity to shorter epitopes and 13mers peptide libraries are shown.

| | | Shortest |
|---|---|---|
| | | region bound |
| scAb clones | Tau proteins/fragments with positive scAb reactivity | on hT40 |
| CA1 | hT40, 1-238, 1-319, 1-155, 155-238 | 155-238 |
| CA2 | hT40, 1-49, 1-319, 1-155, 186-350, 348-441 | 1-155 |
| CA3 | hT40, 1-319, 186-350 | 186-263 |
| CA4 | hT40, 239-441, 297-390 (dGA) | 355-367 |
| CA6 | hT40, 239-441, 348-441 | 348-390 |
| CA7 | hT40, 1-319 | 1-319 |
| CA8 | hT40, 1-319 | 1-319 |
| CA9 | hT40, 239-441, 348-441, 297-390 (dGA) | 367-379 |
| CA10 | hT40, 1-319 | 227-319 |
| CA11 | hT40, 239-441, 348-441 | 348-441 |
| CA12 | hT40, 297-390 (dGA) | 367-379 |
| CB2 | hT40, 348-441 | 359-391 |
| CB3 | hT40, 297-390 (dGA) | 360-390 |
| CB5 | hT40, 1-319 | 49-113 |
| CB6 | hT40, 1-49, 1-155, 1-319, 113-251 | 1-238 |
| CB7 | hT40, 1-49, 1-155, 1-319 | 13-25 |
| CB9 | hT40, 239-441 | 239-348 |
| CB10 | hT40, 1-49, 1-319 | 1-319 |
| CB11 | hT40, 1-319 | 113-238 |
| CB12 | hT40, 1-49, 1-155, 1-319, 113-251, 186-350, 266-359 (R1-3), 297-441 | 115-227 |
| CC3 | hT40, 1-49, 1-155, 1-319, 186-350 | 155-227 |
| CC4 | hT40, 1-155, 1-319 | 49-155 |
| CC5 | hT40, 1-49, 1-155, 1-319, 113-251, 186-350 | 49-238 |
| CC7 | hT40, 1-319 | 145-157 |
| CC12 | hT40, 239-441, 297-390 (dGA) | 297-390 |
| CD1 | hT40, 1-49, 1-155, 1-319, 113-251, 186-350, 239-441, 266-359 (R1-3), 297-441 | 49-155 |
| CD2 | hT40, 1-319, 113-319, 113-251, 186-350 | 186-350 |
| CE2 | hT40, 266-359 (R1-3) | 319-331 |
| CE3 | hT40, 266-359 (R1-3) | 331-360 |
| CG11 | hT40, 186-350, 266-359 (R1-3) | 266-359 |

TABLE 18
A summary of ‘3a’ & ‘3b’ series scAbs binding to various
tau truncations and protein fragments representing regions of
the tau molecule. A further narrow down of binding regions achieved
for some scAbs, including those showing positive reactivity to
shorter epitopes and 13mers peptide libraries are also shown.

| | Tau proteins/fragments with | Short epitope |
|---|---|---|
| scAb clones | positive scAb reactivity | on hT40 |
| 3aD3 | 1-155, 1-111, 1-49 | 1-49 |
| 3aH6 | 1-155, 1-111, 1-49 | 1-49 |
| 3aG3 | 1-155, 1-111, 1-49 | 1-49 |
| 3bG4 | 1-155, 1-111, 1-49 | 1-49 |
| 3aB7 | 1-155, 1-111 | 49-111 |
| 3bF4 | 1-155, 1-111 | 49-111 |
| 3aA6 | 1-155, 113-251, 145-157 | 147-157 |
| 3aD6 | 1-155, 113-251, 145-157 | 147-157 |

### Example 8: Determination of Critical Binding Residues on Selected scAbs Using Alanine Scanning Mutagenesis (ASM)

To further elucidate epitopes of the scAb panel, and identify the critical amino acids needed for binding, alanine scanning mutagenesis of the parent 13 aa peptides for scAbs CE2, S1D12, CA4 and S1G2 was performed. For clones CE2 and CA4, no reactivity was observed for the first and last three overlapping amino acids in the 13mer peptide library which was used for epitope mapping and therefore only the 7 core amino acid sequences were subjected to alanine substitution for these antibodies. Briefly, 5 μg/ml streptavidin (Thermo Fisher) was adsorbed to Nunc 96-well MaxiSorp plates and following incubation at 37° C. for 1 h, the plates were washed as normal and blocked with 2% MPBS. N-terminally biotinylated peptides (Prolmmune Ltd) were added to the plates and incubated for 1 h at 37° C. Subsequently, test scAbs at starting concentrations of 100-500 nM was added and double diluted across the plate for each peptide and incubated for 1 h at 37° C. Rest of the ELISA was conducted as described above and the plates were read at absorbance 450 nm. Immunoreactivity of the scAb of interest was quantified as the % of scAb was bound for each peptide at a chosen concentration (FIG. 15-20).

### Example 9: CE2 Binding Region on hT40: 319-331

TABLE 19
Amino acid sequences of the parent peptide and
alanine substituted peptides for identifying
the critical binding sequences for CE2 scAb

| ASM positions | Peptide Sequences |
|---|---|
| Parent | TSKCGSLGNIHHK |
| 322A | TSKAGSLGNIHHK |
| 323A | TSKCASLGNIHHK |
| 324A | TSKCGALGNIHHK |
| 325A | TSKCGSAGNIHHK |
| 326A | TSKCGSLANIHHK |
| 327A | TSKCGSLGAIHHK |
| 328A | TSKCGSLGNAHHK |

### Example 10: S1D12 & ME12 Binding Region on hT40: 341-353

TABLE 20
Amino acid sequences of the parent peptide and
alanine substituted peptides for identifying the
critical binding sequences for S1D12 scAb and
ME12 scAb

| ASM positions | Peptide Sequences |
|---|---|
| Parent | SEKLDFKDRVQSK |
| 341A | AEKLDFKDRVQSK |
| 342A | SAKLDFKDRVQSK |
| 343A | SEALDFKDRVQSK |
| 344A | SEKADFKDRVQSK |
| 345A | SEKLAFKDRVQSK |
| 346A | SEKLDAKDRVQSK |
| 347A | SEKLDFADRVQSK |
| 348A | SEKLDFKARVQSK |
| 349A | SEKLDFKDAVQSK |
| 350A | SEKLDFKDRAQSK |
| 351A | SEKLDFKDRVASK |
| 352A | SEKLDFKDRVQAK |
| 353A | SEKLDFKDRVQSA |

### Example 11: CA4 Binding Region on hT40: 355-367

TABLE 21
Amino acid sequences of the parent peptide and
alanine substituted peptides for identifying
the critical binding sequences for CA4 scAb

| Peptide | Sequence |
|---|---|
| Parent | GSLDNITHVPGGG |
| 358A | GSLANITHVPGGG |
| 359A | GSLDAITHVPGGG |
| 360A | GSLDNATHVPGGG |
| 361A | GSLDNIAHVPGGG |
| 362A | GSLDNITAVPGGG |
| 363A | GSLDNITHAPGGG |
| 364A | GSLDNITHVAGGG |

### Example 12: Clones with Binding Region 367-379 on hT40

Several antibody clones were shown to bind to the region 367-379 on hT40 molecule (Table 16 & 17) and were grouped together and subjected to detailed analysis of critical binding residues using ASM peptides as described above. Amino acid sequences of the parent peptide and alanine substituted mutants are given in Table 22. Binding profiles of S1G2 scAb to the parent peptide and mutants are shown in FIG. 19A-C. Similarly, percentage binding of other scAbs in the same group recognising hT40 367-379 to various alanine substituted mutants and the parent peptide are shown in FIG. 20A-J

TABLE 22
Amino acid sequences of the parent peptide and
alanine substituted peptides for identifying
the critical binding sequences for S1G2 and
related scAbs binding to 367-379 region

| Peptide | Sequence |
|---|---|
| Parent | GNKKIETHKLTFR |
| 367A | ANKKIETHKLTFR |
| 368A | GAKKIETHKLTFR |
| 369A | GNAKIETHKLTFR |
| 370A | GNKAIETHKLTFR |
| 371A | GNKKAETHKLTFR |
| 372A | GNKKIATHKLTFR |
| 373A | GNKKIEAHKLTFR |
| 374A | GNKKIETAKLTFR |
| 375A | GNKKIETHALTFR |
| 376A | GNKKIETHKATFR |
| 377A | GNKKIETHKLAFR |
| 378A | GNKKIETHKLTAR |
| 379A | GNKKIETHKLTFA |

### Example 13: Ranking of Relative Binding Affinities of Anti-Tau scAbs Using hT40 Binding ELISA

The relative binding affinities of anti-tau scAbs were ranked by performing hT40 antigen binding ELISA as described previously.

### Example 14: Analysis of the Binding Kinetics of Anti-Tau scAbs Using Biacore X100™

Surface plasmon resonance (SPR) is widely regarded as the gold standard for real time measurement of protein-protein interactions such as antibody binding. All SPR experiments were carried out using a Biacore X100 machine and HBS EP+ running buffer (GE Healthcare). A ‘capture’ method was followed for affinity measurements, where an anti-human constant kappa chain (HuCk) antibody was conjugated to the surface of a CM5 sensor chip using an amine coupling kit and scAb molecules were immobilised via their HuCk domain. Amine coupling is a very common approach for immobilising the ligand to the chip surface. The chip surface has a dextran matrix derivatised with carboxyl groups, which after activation with N-hydroxysuccinimide (NHS) and 1-ethyl-3-(3-dimethylaminopropyl) carbodiimide (EDC), form reactive succinimide esters which allows the covalent capturing of the ligand via any available primary amine groups (e.g. Lysine) on the ligand (in this case anti-HuCk antibody). The capture antibody was diluted 1/100 in 10 mM Sodium acetate buffer pH 5.0 and passed over the activated chip surface for a period of at least 420 s. Final ligand immobilisation levels over 12,000 RU were deemed to be satisfactory.

Following immobilisation, the level of each scAb to be captured was identified using the standard SPR equation with a theoretical RMax of 100 RU. Analyte MW refers to the molecular weight of hT40 or dGA/dGAE molecules, while ligand MW refers to the molecular weight of the scAbs tested. RLis the desired capture level and S refers to the stoichiometric ratio:

$$Rmax = \frac{Analyte M W}{ligand M W} \times Rl \times S$$

The scAb to be tested was added only to flow cell 2 so that flow cell 1 could act as a control to subtract any interaction with the analyte and the chip surface. Both single and multi-cycle runs were utilised using wizards developed in the Biacore X100 control software which are both accepted methods of kinetic analysis (Karlsson, R., et al. Analyzing a kinetic titration series using affinity biosensors (2006) Analytical Biochemistry 349:136-47). The multi-cycle protocol was as follows-Three start-up cycles composed of an initial capture, to the desired level, of the scAb to be tested was performed followed by a 30 s injection of glycine buffer pH 2.0 after each cycle to regenerate the chip surface. The regeneration step removed any captured scAb whilst leaving the capture antibody intact and able to repeat the capture for the next cycle. Following three start-up cycles increasing concentrations (0.15625 nM-100 nM) of the target to be tested were added to the chip surface after the desired scAb capture. The target was added for a 120 s association period and 420 s dissociation period followed by another regeneration, 30 s injection of glycine buffer pH 2.0 in between each cycle.

The single cycle kinetics protocol was similar and utilised the same level of scAb capture. Three start-up cycles were used followed by 5 increasing concentrations of the analyte (6.25 nM-100 nM) for an association period of 120 s followed by a dissociation period of 420 s for each concentration. In single cycle kinetics the regeneration step was performed only after the final analyte concentration was added. Binding responses were analysed in Biacore X100 evaluation software and data fitted to a 1:1 binding model to obtain kinetic and affinity characterisation. Kinetic rates and equilibrium binding constants of lead scAbs are given in Table No: 23-26.

TABLE 23
Kinetic rates and equilibrium binding
constants of lead scAbs for T441

| Top clones | | | kD (M) for | |
|---|---|---|---|---|
| (scAbs) | ka (1/Ms) | kd(1/s) | hT40 binding | |
| S1D12 | 1.252 × 10⁶ | 1.5 × 10⁻⁴ | 122 | pM |
| S1G2 | 1.057 × 10⁶ | 1.8 × 10⁻⁴ | 170 | pM |
| S1E12 | 3.85 × 10⁵ | 3.19 × 10⁻⁴ | 829 | pM |
| CC7 | 2.957 × 10⁵ | 3.659 × 10⁻⁴ | 1.23 | nM |
| NS2A1 | 2.521 × 10⁵ | 7.393 × 10⁻⁴ | 2.9 | nM |
| CA4 | 4.581 × 10⁵ | 1.654 × 10⁻³ | 3.61 | nM |
| CE3 | 1.745 × 10⁵ | 6.620 × 10⁻⁴ | 3.79 | nM |
| 412-E10 | 2.71 × 10⁵ | 8.5 × 10⁻⁴ | 3.16 | nM |
| CE2 | 2.137 × 10⁵ | 1.077 × 10⁻³ | 5.03 | nM |
| CB7 | 1.334 × 10⁵ | 6.936 × 10⁻⁴ | 5.19 | nM |
| E1B8 | To be determined | | | |
| E2E8 | No T441 binding | | | |
| MoD9 | To be determined | | | |

TABLE 24
Kinetic rates and equilibrium binding
constants of lead scAbs for dGA or dGAE

| Top clones | | | kD (M) for truncated | |
|---|---|---|---|---|
| (scAbs) | ka (1/Ms) | Kd(1/s) | tau binding | |
| S1D12 | 1.21 × 10⁶ | 4.18 × 10⁻⁴ | 344 | pM (dGA) |
| S1G2 | 7.83 × 10⁵ | 3.499 × 10⁻⁴ | 447 | pM (dGA) |
| S1E12 | 2.99 × 10⁶ | 1.627 × 10⁻³ | 543 | pM (dGA) |
| NS2A1 | 1.88 × 10⁵ | 1.02 × 10⁻³ | 5.4 | nM (dGA) |
| E1B8 | 1.37 × 10⁵ | 2.42 × 10⁻³ | 17.7 | nM (dGA) |
| E2E8 | 4.77 × 10⁵ | 1.915 × 10⁻⁴ | 401 | pM (dGAE) |
| E1E8 | 1.535 × 10⁵ | 1.4 × 10⁻³ | 6.3 | nM (dGAE) |
| MoD9 | To be determined | | | |
| CE2 | To be determined | | | |

TABLE 25
Kinetic rates and equilibrium binding constants of
‘3a’ and ‘3b’scAbs for hT40 (nM)

| scAb | | | kD (nM) for | |
|---|---|---|---|---|
| clones | ka (1/Ms) | kd(1/s) | htau40 binding | |
| 3aA6 | 1.900 × 10⁴ | 1.880 × 10⁻² | 989 | nM |
| 3aB7 | 2.072 × 10⁵ | 1.435 × 10⁻² | 69 | nM |
| 3aD3 | 5.444 × 10⁵ | 1.038 × 10⁻² | 19.1 | nM |
| 3aD6 | 4.021 × 10⁵ | 6.641 × 10⁻³ | 16.5 | nM |
| 3aH6 | 2.757 × 10⁶ | 9.868 × 10⁻³ | 3.6 | nM |
| 3aG3 | 6.294 × 10⁴ | 3.869 × 10⁻⁴ | 6.1 | nM |
| 3bA3 | 1.472 × 10⁴ | 4.643 × 10⁻³ | 315 | nM |
| 3bC1 | 3.056 × 10⁴ | 4.231 × 10⁻³ | 138 | nM |
| 3bF4 | 1.093 × 10⁵ | 1.532 × 10⁻² | 140 | nM |
| 3bG4 | 6.669 × 10⁶ | 5.966 × 10⁻² | 8.9 | nM |

TABLE 26
Summary of equilibrium binding constants
and binding regions of top anti-tau scAbs

| Top clones (scAbs) | Epitope | kD for hT40 binding | |
|---|---|---|---|
| CB7 | 13-25 | 5.19 | nM |
| CC7 | 145-157 | 1.23 | nM |
| NS2A1 | 297-390 | 2.9 | nM |
| S1E12 | 297-390 | 829 | pM |
| E1B8 | 316-336 | Not available | |
| CE2 | 319-331 | 5.03 | nM |
| CE3 | 331-360 | 3.79 | nM |
| S1D12 | 337-349 | 122 | pM |
| CA4 | 355-367 | 3.61 | nM |
| S1G2 | 367-379 | 170 | pM |
| MoD9 | 373-385 | | |
| E2E8 | 391 | No hT40 binding | |
| 412-E10 | 412-441 | 3.16 | nM |

### Example 15: IgG Reformatting

Top anti-tau scAbs were reformatted into sheep-mouse (IgG2a) chimeric mAbs by inserting respective VH and VL genes into our dual plasmid eukaryotic vector system (pEE2a) encoding constant heavy and light chain genes of mouse IgG2a separately and expressing recombinant mAbs in a mammalian expression system. Based on the DNA sequencing data, VH and VL genes of shortlisted anti-tau scAbs were custom synthesised separately by introducing the cloning sites BssHII and BstEII (for VH genes) and BssHII and XhoI (for VL genes) at their 5 and 3′ end respectively (GeneArt custom gene synthesis service by Thermofisher). Custom synthesised VH and VL genes of respective scAbs and the eukaryotic expression vectors pEE2aMH (encoding mouse IgG2a constant regions) and pEE2aML (mouse N/K constant domain) were digested with restriction enzymes mentioned above. DNA bands corresponding to antibody variable regions and pEE2a heavy and light chain vector backbones were isolated and purified using DNA gel extraction and purification following QIAquick gel extraction kit manufacturer's instructions. Purified DNA fragments were ligated and used to transform electrocompetent E. coli TG1 cells for plasmid propagation. DNA sequencing of extracted plasmid confirmed successful reformatting into sheep-mouse chimeric mAbs. Large scale preparation of heavy and light chain plasmids for each anti-tau mAb clone was performed (Qiagen Plasmid Mega kit) and used to transfect Human Embryonic Kidney (HEK293F) cells grown in suspension using polyethylenimine (PEI). The transfected cells were grown for 8 days before harvesting cell culture supernatants which were then purified using Protein A beads following standard protocols. Purified mAbs were confirmed for T40 binding using ELISA and their affinities (kD) values obtained by running Biacore assays as described previously, with changes as described below.

### Example 16: Analysis of the Binding Kinetics of Anti-Tau mAbs Using Biacore X100™

For anti-tau mAb SPR measurements, htau40 was conjugated to the surface of a CM5 sensor chip using amine coupling as explained previously. Briefly, 2 μg/mL htau40 in 10 mM Sodium acetate buffer (pH 4.0) was passed over the activated chip surface (EDC/NHS) in flow cell 2 for a period for 45 s followed by blocking with 1 M ethanolamine-HCl pH 8.5. Flow cell 1 was set up as reference control and blocked simultaneously. Final immobilisation level of htau40 (Rmax) was approximately 250 RU. Following a minimum of three start-up cycles, increasing concentrations (0.78 nM-25 nM) of the mAb in HBS-EP+ buffer was added at a rate of 30 μL/min to the chip. The mAb was added for a 120 s association period and 600 s dissociation period followed by a regeneration cycle of 30 s injection of glycine buffer pH 1.5 in between each cycle. Binding responses were analysed in Biacore X100 evaluation software and data fitted to a 1:1 binding model to obtain kinetic and affinity characterisation. Kinetic rates and equilibrium binding constants of lead anti-tau mAbs are given in the table below

TABLE 27
Kinetic rates and equilibrium binding constants of
lead anti-tau mAbs for hT40 (TBD, to be determined)

| Top clones | | | kD (M) for | |
|---|---|---|---|---|
| (mAbs) | ka (1/Ms) | kd(1/s) | hT40 binding | |
| S1D12 | 4.058 × 10⁵ | 4.093 × 10⁻⁵ | 101 | pM |
| S1G2 | 3.398 × 10⁵ | 4.748 × 10⁻⁵ | 140 | pM |
| S1E12 | TBD | TBD | TBD | |
| CC7 | 4.576 × 10⁶ | 3.164 × 10⁻² | 6.9 | nM |
| NS2A1 | 8.937 × 10⁵ | 2.621 × 10⁻³ | 10 | nM |
| CA4 | 1.020 × 10⁶ | 1.276 × 10⁻³ | 1.3 | nM |
| CE3 | 1.030 × 10⁵ | 1.718 × 10⁻³ | 16.7 | nM |
| 412-E10 | 1.658 × 10⁶ | 1.497 × 10⁻² | 9.0 | nM |
| CE2 | 4.579 × 10⁵ | 5.218 × 10⁻³ | 11 | nM |
| CB7 | 2.568 × 10⁶ | 9.451 × 10⁻³ | 3.7 | nM |

### Example 17: Determination of Limits of Detection (LoD) for High Affinity Anti-Tau Antibody Pairs

The diverse panel of high affinity anti-tau antibodies (scAb and mAb formats) were paired in various combinations and tested in a Sandwich (or Capture) ELISA format to calculate their limits of detection (LoD) and their ability to differentiate between different tau species. Biologically, tau exists in 6 different isoforms and is subjected to numerous post translational modifications, some of them playing a significant role in the progression of neurodegeneration.

For Sandwich ELISA using colourimetric detection, 96 well Maxisorp plates were coated with the capture antibody S1D12 mAb at 1 μg/ml for 1 h at RT followed by blocking with 2% MPBS as normal. Full length tau (hT40), at 1 μg/ml starting concentration was added to designated wells and double diluted across rest of the plates and left to incubate at R/T for 1 h. A series of detection scAbs with varying epitope recognition properties were added at 10 μg/ml to designated wells (FIG. 22). For colourimetric detection HRP conjugated anti-HuCk secondary antibody was used and the resulting immunoreaction was developed and absorbance at 450 nm measured as described previously. Cut-off point for a positive binding event was an absorbance value of 0.3 following background subtraction. In order to enhance the assay sensitivity, a chemiluminescent detection method was adopted where 50 μL SuperSignal ELISA Femto Substrate (Thermo Scientific) was added to each well following incubation with anti-HuCK HRP antibody and subsequent washing. The luminescence was read using Clariostar Plus microplate reader. A further modification to this protocol was done where various detection scAbs were directly conjugated with HRP and detected using chemiluminescent protocol as before. The lowest levels of detection achieved using different detection scAbs and a comparison of colourimetric and chemiluminescent methods of detection are shown in Table 28

TABLE 28
The LoDs achieved using S1D12 mAb capture and various
detection scAbs and a comparison of colourimetric
and chemiluminescent methods of detection

| | | | | LoD (ng/ml) |
|---|---|---|---|---|
| | | LoD | LoD | direct HRP |
| | | (ng/ml) | (ng/ml) | conjugation |
| | | col- | chemi- | of detection |
| | Tau-441 | ourimet- | lumines- | scAb & |
| Detection scAb | affinities | ric | cence | chemilumi- |
| (Epitope) | (Biacore) | detection | detection | nescence |
| CB7 (13-25) | 5.2 (nM) | 15 | 3.33 | |
| CA2 (100-155) | — | 6 | 0.37 | |
| CC7 (145-157) | 1.2 (nM) | 6 | 3.33 | |
| CA4 (355-367) | 3.6 (nM) | 6 | 0.37 | |
| S1G10 (367-379) | 0.4 (nM) | 2 | 0.12 | |
| CB8 (367-379) | 0.6 (nM) | 2 | 1.11 | |
| S1G2 (367-379) | 0.6 (nM) | 2 | 0.12 | 0.04 |
| 412E10 (412-441) | 3.3 (nM) | 20 | 0.37 | |
| E2E8 (E | 0.4 (nM) | — | | |
| dependant) | dGAE | | | |

Similarly, a sandwich ELISA format was set up using S1G2 mAb at 1 μg/ml as the capture antibody and HRP conjugated S1D12 mAb for detection. The assay was conducted using chemiluminescent protocol described above. The assay setup and quantification of the limit of detection are shown in FIGS. 23 and 24

### Example 18: Interrogation of Mixed Samples and their Quantification Using Antibody Pairs

The ability to interrogate and determine the levels of various tau species or fragments in patient samples is crucial in early AD diagnosis. To this end, an experiment was set up to assess whether the concentrations of different tau species could be determined in spiked samples using various antibody pairings directed towards specific regions of the tau protein. Four spiked samples were prepared with varying concentrations and types of tau species-sample A with 5 nM full length human tau (hT40), sample B with 3.3 nM dGA, 3.3 nM dGAE and 3.3 nM hT40 (9.9 nM total protein), sample C with 2 nM dGA, and sample D with 1 nM hT40 and 4 nM dGA (5 nM total protein). These samples were analysed by performing three separate ‘blind’ sandwich ELISAs using S1D12 mAb to capture the different species in the mixture and detection using scAbs with specific epitopes.

For ELISA #1, wells were coated with S1D12 mAb, blocked and 20 nM hT40 was added to the first well for standard curve plotting. Four spiked samples were added to the first wells of designated rows and all samples were double diluted in PBS across the plate including the standard. The samples were incubated at RT for 1 h, washed as normal and 1 μg/ml CB7 scAb was added to each well and incubated as before. Secondary antibody anti HuCk HRP was added and the reaction was developed and read as described previously. For ELISA #2, 20 nM dGAE was added to the first well and double diluted across the plate for standard curve plotting. As for ELISA #1, spiked samples of unknown protein concentrations were added to respective wells in doubling dilutions. The detection antibody used was ‘E’ specific E2E8 scAb and rest of the ELISA was performed as described above. For ELISA #3, a standard curve was plotted using average absorbance values obtained from binding events of hT40, dGA and dGAE all at starting concentration of 20 nM. As before, four spiked samples were tested for binding using S1D12 capture mAb and core binding S1G2 detection scAb. Based on ELISA signals of unknown spiked samples, the types of tau fragments present in the mixture and their individual concentrations were determined as shown in FIGS. 25, 26 and 27. A summary of tau types and their concentrations used to spike these samples and the deduced concentrations from ELISAs are given in Table 29.

TABLE 28
Summary of various tau species and their concentrations in the spiked
samples and their deduced concentrations and reactivity using three
different antibody pairings in a sandwich ELISA format.

| | | ELISA #1 | ELISA #2 | ELISA #3 | |
|---|---|---|---|---|---|
| | | S1D12-CB7 | S1D12-E2E8 | S1D12-S1G2 | |
| | | Reactivity & | Reactivity & | Reactivity & | |
| | Concentrations of | deduced | deduced | deduced | |
| | various tau species in | concentration | concentration | concentration | |
| Samples | the sample mix | of samples | of samples | of samples | |
| A | 5 nM hT40 | 5 nM N terminal | None | 3 | nM core |
| B | 3.3 nM hT40 + 3.3 nM | 4 nM N terminal | 3.2 nM ‘E’ | ~9 | nM core |
| | dGA + 3.3 nM dGAE | | specific | | |
| C | 2 nM dGA | None | None | 1 | nM |
| D | 1 nM hT40 + 4 nM dGA | 1.5 nM N terminal | None | 3.2 | nM core |

### Example 19: SDS Treatment of dGAE Aggregates and Restoration of Immunoreactivity

The immunoreactivity of core region scAbs is lost when dGA/dGAE fragments are aggregated which could be due to the non-availability of respective epitopes in this conformation. We have noticed Sodium dodecyl sulphate (SDS) can break apart dGA/dGAE aggregates and separate them into smaller fragments by performing an SDS-PAGE. This has been replicated and tested using an ELISA as described below.

For aggregation, 1000 μL 100 μM dGAE+10 μL 10 mM DTT was added to a ‘LoBind’ microfuge tubes and agitated at 700 RPM/37° C. for 24 hours. Resulting sample centrifuged at 17,000×g/4° C. for 60 min and supernatant discarded to remove left over monomer. Pellet resuspended in half the original volume for future experiments and hereafter referred to as ‘aggregates’. 1 μl of aggregates was added to 1 ml of PBS and SDS was added to a final concentration of 1% (w/v). This was left to incubate on lab bench for 1 h with gentle agitation every 15 minutes. In order to neutralise the SDS effect on ELISA, Triton X-100 added to a final concentration of 3% (v/v) and mixed gently by pipetting to prevent any bubble formation. 200 μl added of this mix was to the first well of an ELISA plate coated with 1 μg/ml S1D12 mAb and blocked with 2% MPBS. Similarly aggregates treated with only SDS or Triton X-100, untreated aggregates, dGAE monomers treated with only SDS or SDS+Titon X-100 were also added to designated wells as controls. All samples were then double diluted across the plate in final volumes of 100 μl. The final column was left with no protein to act as a blank. This was allowed to stand at room temperature for 1 hour followed by the addition of detection scAb-S1G2 at 10 μg/ml. Anti-HuCk HRP labelled secondary antibody was added as described previously and ELISA data generated was represented using the graph below. In addition, limits of detection (LoD) of various core binding antibody pairing for SDS Triton X-100 treated dGAE aggregates were calculated using the above describe ELISA method and substituting capture mAbs and detection scAbs as shown in Table 30.

Table 30 shows the limits of detection (LoD) of various capture mAb-dection scAb pairing for SDS Triton X-100 treated dGAE aggregates in a sandwich ELISA system. NB denotes no binding

| | Capture System | | | | | |
|---|---|---|---|---|---|---|
| | | | | | Direct immobilisation of | |
| | S1D12 mAb | | 423 mAb | | dGAE aggregates | |
| Detection | Untreated | +SDS Triton | Untreated | +SDS Triton | Untreated | +SDS Triton |
| scAb | dGAE agg. | dGAE agg. | dGAE agg. | dGAE agg. | dGAE agg. | dGAE agg. |
| S1G2 scAb | 250 ng/ml | 2 ng/ml | NB | 30 ng/ml | NB | 16 ng/ml |
| CA9 scAb | | 4 ng/ml | | | | 40 ng/ml |
| CA12 scAb | | 2 ng/ml | | | | 16 ng/ml |
| CB8 scAb | | 4 ng/ml | | | | 30 ng/ml |

### Example 19: Antibody Based Detection of Various Tau Fragments in Transgenic Mice Brain Lysate Collection

Mouse brain lysates were prepared from wildtype, Line 1, Line 66+/+ and Line 66+/− (Melis et al., 2015, all surplus from Charles River, part of study R0144). All animals were female, 7-8 months old except Line 66+/+ which were 5 months old. The Line 66 mouse construct (FIG. 23) contains human tau cDNA coding for the longest human tau isoform (2N4R tau; 441 amino acids) with two point mutations at P301S and G335D. The L1 cDNA construct (FIG. 23) contains human tau cDNA coding for amino acid residues 296-390 with a signal sequence and related sequences in a murine Thy-1 expression cassette. For brain lysate preparation, half brains were sectioned into four and the individual sections were homogenised in 400 μl of ice cold RIPA buffer (Cell signalling Technology) containing protease and phosphatase inhibitors. Total protein concentrations for individual samples were established by BCA-assay and each sample was subsequently diluted to 1 mg/ml for future experiments.

For capture ELISA, 100 μL S1D12 capture mAb was used to coat a nunc 96 well Maxisorp plate at 1 μg/ml and incubated for 1 hour at 37° C. Plate was washed as previously and then blocked for 1 hour at 37° C. in 2% MPBS. Brain homogenate samples (50 ng total protein) from each mouse type were added to the first wells and double diluted across the plate in PBS. These samples were left to incubate at RT for 1 hour. Various detection scAbs encompassing epitopes of interest were then added to the plate at 1 μg/ml and incubated for 1 h at RT. Secondary antibody used was anti-HuCk HRP and the assays were developed as described previously. Using S1D12 mAb capture and two separate scAb detectors, S-1G2 (FIG. 24A) and C-B7 (FIG. 24B), it was possible to differentiate between L1 and L66 samples and the wild type. When s1G2 scAb was used, tau protein was detected in 50 μg of total brain protein in all four different sample types. However, with C-B7 scAb, hT40 tau was specifically detected in L66 samples and an increase was observed in L66 homozygous group compared to L66 heterozygous samples. Therefore S1D12-CB7 pair was able to differentiate L66 from L1 and wild type with the use of an N′ terminally directed detection scAb which is due to a difference in sequence homology between mouse and human tau (Hernandez et al., 2019). Over the whole protein there is approximately 77% homology at the amino acid level, but the proteins differ significantly in the N′-terminal region.

### Example 20: Antibody Based Detection of Tau Fragments in Transgenic Mice Plasma Collection

Plasma samples from WT, L1, L66+/− and L66+/+ of various ages (1.5-9 months) were taken as described. Mice were terminally anaesthetized using an overdose of sodium pentobarbital and blood was collected through cardiac puncture through a Plastipak syringe pre-rinsed with heparinised saline heparin (10 U/ml) (Heparin sodium salt; Sigma-Aldrich) and transferred into a plastic vial containing lithium heparin anticoagulant (Sarstedt Ltd.) Blood samples, kept on ice for no more than 30 minutes, were centrifuged to obtain plasma at 2000×g for 5 minutes at 6° C. Plasma samples were stored at −20° C.

For mouse plasma capture ELISA, 100 μL capture mAb was coated to the bottom of a black nunc 96 well ‘maxisorp’ plate at 2.5 μg/ml and incubated for 1 hour at 37° C. After washing and blocking as normal, several known standard solutions were set up in triplicate starting at 20 ng/ml of the desired protein before double diluted in the remaining wells. Mouse samples were added to wells in duplicate with a blank in between each sample group. Line1 plasma was diluted 1:10 before addition to plate to account for its high concentration while other samples were diluted 1:2 to account for potential matrix effects. These samples were left to incubate at RT for 1 hour. HRP-conjugated secondary scAb (conjugation carried out according to manufacturer's guidelines, Abcam) was added to each well and left to incubate at RT for 1 hour. The ELISA was developed using SuperSignal ELISA Femto Substrate (Thermo Scientific) and total luminescence was read on Clariostar Plus plate reader (BMG Labtech). Tau concentrations were established using a 4-parameter fit on concentration curves generated from recombinant tau protein spiked into samples at known concentration. For WT and L66 hT40 was used as a calibrator while dGA was used for L1 calibration.

Using S1D12 capture and S1G2 detection we were able to detect varying levels of ‘core region’ containing tau fragments in samples from WT, L1, L66+/− and L66+/+. In WT and L66, the levels detected were in low ng/ml (WT at 5 month: 1.947 ng/ml, WT at 9 month: 2.177 ng/ml); (L66+/− at 5 month: 0.567 ng/ml), L66+/+ at 5 month: 1.937 ng/ml). However higher concentrations of core region tau species were detected in L1 samples (5 month: 12.355 ng/ml, 9 month 13.661 ng/ml). This is in agreement with the genetic makeup of L1 mouse which contains a truncated 3 repeat fragment corresponding residues 296-390 of hT40 and a signal sequence that drives this towards the endoplasmic reticulum. This could explain the presence of elevated levels of repeat domain core containing fragments in L1 mouse plasma that was detected using our core region pair, S1D12 mAb and S1G2 scAb. This region also shares sequence homology with mouse tau protein which is reflected by base level detection of the core in WT, L66+/+ and L66+/− plasma samples.

In addition, using a second capture-detection antibody pairing, we have successfully shown the detection of human specific tau fragments in Line66 mice which express the longest tau isoform (hT40, 441 amino acids) containing 4 repeat regions with point mutations P301S and G335D (Melis et al., 2014). Using S1D12 mAb as the capture antibody and CB7 scAb detection, Line 66+/+ mouse plasma at 1.5 months showed the presence of detectable levels of human tau as compared to the wild type mouse of same age (FIG. 32A). The detection scAb CB7 (epitope 13-25 on hT40) does not cross react with endogenous mouse tau protein in Line66 transgenic model since the N terminal amino acid sequences differs considerably with between human and mouse tau. In addition, using S1D12-S1G2 pairing we have shown similar levels of core repeat region tau fragments which share close sequence homology in transgenic and wild type animals (FIG. 32B) This demonstrates the utility of our panel of antibody pairs in detecting various tau fragments and truncations in transgenic mice samples which can be translated into a diagnostic setting to interrogate the presence of different tau species in diseased vs cognitively normal human samples.

### Example 21: Antibody Based Detection of Tau Fragments in Plasma Samples from AD Patients

Six plasma samples from individuals with an AD diagnosis and 6 samples from aged matched controls were sourced (Logical Biological, Kent UK), divided into 100 μl aliquots and stored at −80° C. (Table 31)

TABLE 31
Demographic characteristics of human plasma samples used in this
study including the age, sex and ethnicity of cognitively normal
controls and patients with confirmed AD diagnosis. Mini-Mental
Sate Examination (MMSE) scores of AD patients are also shown.

| | Donor | | | | |
|---|---|---|---|---|---|
| | Number | Age | MMSE | Sex | Ethnicity |
| Age- | PL 361 | 64 | — | F | Caucasian |
| Matched | PL 362 | 61 | — | M | Caucasian |
| Control | PL 363 | 61 | — | M | Caucasian |
| | PL 364 | 63 | — | M | African American |
| | PL 365 | 61 | — | F | Caucasian |
| | PL 366 | 66 | — | F | African American |
| AD | PL 505 | 62 | 27 | F | Caucasian |
| | PL 506 | 63 | 25 | F | Caucasian Italian |
| | PL 507 | 64 | 26 | M | Hispanic |
| | PL 508 | 57 | 25 | M | African American |
| | PL 509 | 64 | 21 | M | African American |
| | PL 510 | 63 | 27 | F | Caucasian |

For sandwich ELISA, 100 μl S1D12 capture mAb was coated to the bottom of a black nunc 96 well ‘maxisorp’ plate at 2.5 μg/ml and incubated for 1 hour at 37° C. Plate was washed as previously and then blocked for 1 hour at 37° C. in 2% MPBS. Several known hT40 standard solutions were set up in triplicate starting at 8 ng/ml and diluted 2-fold in the remaining wells using 50% sheep plasma as a diluent to account for any plasma matrix effects. 100 μl of each human sample diluted 1:2 in PBS were added to wells in duplicate with a blank in between each sample group. Samples were diluted 1:2 to account for potential matrix effects. These samples were left to incubate at 4° C. overnight. HRP-conjugated secondary scAb ((S-1G2 and C-B7) (conjugation carried out according to manufacturer's guidelines, Abcam)) was added to each well and left to incubate at RT for 1 hour. The ELISA was developed using SuperSignal ELISA Femto Substrate (Thermo Scientific) and total luminescence was read on a plate reader (BMG Labtech). Tau concentrations were established using a 4-parameter fit on concentration curves generated from recombinant human tau protein spiked into samples at known concentration.

Tau was detected in both patient groups (FIG. 33). Interestingly different levels were detected depending on the combination of capture/detection antibody used. This data provides a proof of concept that using different pairs of antibodies depending on their epitopes will allow us to detect different fragments of tau within plasma.

TABLE 32
Summarises plasma tau levels of individual samples in AD and
control groups using the capture-detection pairs as shown

| | | S1G2-HRP | CB7-HRP |
|---|---|---|---|
| | Donor | Detection | Detection |
| | Number | (ng/ml) | (ng/ml) |
| Age- | PL 361 | * | 1.89 |
| Matched | PL 362 | * | 1.04 |
| Control | PL 363 | 0.036 | 1.89 |
| | PL 364 | 0.565 | 1.93 |
| | PL 365 | * | * |
| | PL 366 | 0.74 | 1.84 |
| AD | PL 505 | * | * |
| | PL 506 | * | 6.75 |
| | PL 507 | 2.13 | 2.7 |
| | PL 508 | 1.83 | ** |
| | PL 509 | 0.63 | * |
| | PL 510 | 1.38 | 1.96 |

(* Below assay limit of detection,
** Above assay limit of detection)

### Example 22: LMT Mediated Inhibition of dGAE Aggregates and Restoration of Immunoreactivity

The truncated core repeat domain dGAE (297-391), is the predominant fragment that constitutes bulk of the PHF core in AD (Wischik et al, 1988). During dGAE aggregation in vitro, scAb binding regions on dGA/dGAE are ‘hidden’ or ‘occluded’ which leads to a loss of immunoreactivity in aggregation samples. Here we have shown the occlusion of binding regions in aggregated dGAE samples and the recovery of immunoreactivity in the presence of LMTM, a tau-aggregation inhibitor. The scAbs tested for binding are core region specific S1D12, CA4, CB3, CE2, CE3 and CA9 (binding regions given in Table 22). For preparing the aggregates, 10 μl 10 mM DTT was added to 1000 μl 100 μM dGAE and agitated with/without LMTM (1:5 ratio) at 700 rpm for 24 h at 37° C. Following overnight agitation, one third of each sample was kept aside as ‘total’ and the rest was spun down at 16000×g for 30 min and separated into ‘supernatant’ and ‘pellet’. The pellet was then resuspended in half the original volume for further experiments. The immunoreactivities of core region specific scAbs towards dGAE aggregates formed with/without LMTM was tested using a sandwich ELISA format using a ‘E’ specific monoclonal antibody 423 mAb. This mAb has been shown to specifically bind to the Pronase resistant core structure in the PHFs (Wischik et al, 1988). ELISA plates were coated with 10 μg/ml 423 mAb and blocked as normal. Doubling dilutions of dGAE aggregate ‘total’, ‘supernatant’ and ‘pellet’ samples at 10 μg/ml starting concentration were added to designated wells in doubling dilutions in 1×PBS. dGAE monomer (non-aggregated) was included as assay control. All double dilutions were done in final volumes of 100 μl. This was left to incubate on lab bench for 1 h followed by the addition of test scAbs at 10 μg/ml. Anti-HuCk HRP labelled secondary antibody was added as described previously and ELISA data generated is represented using the graph below.

TABLE 33
Core binding scAbs tested in epitope occlusion assays
and their specific binding regions on Ht40

| scAbs tested | Binding regions on hT40 |
|---|---|
| S-1D12 | 337-355 |
| CA4 | 355-367 |
| C-B3 | 360-390 |
| C-E2 | 319-331 |
| C-E3 | 297-356 |
| C-A9 | 367-379 |

All scAbs tested showed increased binding to aggregated dGAE ‘total’ and ‘supernatant’ samples, when aggregation was conducted in the presence of LMTM. This proves the opening or revealing of occluded antibody binding regions on dGAE where LMTM is preventing the aggregation event, leading to an increased immunoreactivity (FIG. 34).

### Example 23: Ranking of Tau mAbs Based on their Ability to Capture dGAE Aggregates

The aggregation cascade of natively unfolded tau into insoluble filaments is a defining pathological feature of AD. Therefore, it is logical to target aggregated tau filaments for both therapeutic and diagnostic purposes. The following experiment was performed to assess the ability of our dGA mAb panel to bind aggregated dGAE using a capture ELISA method. Aggregates were prepared by incubating 100 μM dGAE and 10 mM dTT for 24 h at 37° C. with shaking at 700 RPM. The following day, dGAE aggregates were centrifuged at 17,000×g for 20 min and the supernatant was removed. The remaining pellet was washed with 10 mM phosphate buffer and centrifuged as above. Washing was repeated a further two times to remove any remaining dGAE monomer. The dGAE pellet was resuspended in 100 μl of 10 mM phosphate buffer. (Using a separate capture ELISA based quantification, we have worked out the efficiency of our dGAE aggregation as 80%) For assessing the mAb panel for aggregate binding, designated rows of a maxisorp plate were coated with 1 μg/ml of each of the capture mAbs (S1D12, S1G2, CA4, NS2A1, CE2, E2E8 and CB7) and blocked as before. Following washing, 800 nM aggregated dGAE was added to the designated wells, double diluted across the plate and incubated for 1 h at RT. The plate was washed again and 1 μg/ml S1G2 scAb was added as detection antibody and incubated for 1 h at RT. To the row where S1G2 mAb was used for capture, 1 μg/ml s1D12 scAb was added as capture antibody instead of S1G2. The assay was developed using HRP labelled HuCK as described previously.

Capture ELISA graph indicates that S1D12 and S1G2 mAbs are most efficient at capturing aggregated dGAE, along with E2E8 mAb which like the 423 mAb is a ‘391E’ binder (see FIG. 35).

### Example 24: Epitope Mapping and Binding Affinities of Reformatted Anti-Tau mAbs

For anti-tau mAb SPR measurements (Biacore X100™) and epitope mapping, the same methodologies described above (see Example 16) were followed. Kinetic rates and equilibrium binding constants of additional anti-tau mAbs and the regions recognised on hT40 are given in the Table 25 below

TABLE 25
Kinetic rates, equilibrium binding constants and
binding regions of newly converted anti-tau mAbs.

| Clones | ka | | kD (M) for hT40 | | hT40 binding |
|---|---|---|---|---|---|
| (mAbs) | (1/Ms) | kd(1/s) | binding | | region |
| 3aH6 | 3.128 × 10⁶ | 1.522 × 10⁻⁴ | 49 | pM | 1-15 |
| 3bG4 | 4.540 × 10⁶ | 2.964 × 10⁻⁴ | 65 | pM | 1-15 |
| 3aG3 | 1.532 × 10⁷ | 4.751 × 10⁻³ | 310 | pM | 1-15 |
| 3bD11 | 2.671 × 10⁶ | 3.979 × 10⁻³ | 1.490 | nM | 37-49 |
| E1B8 | 3.238 × 10⁵ | 1.367 × 10⁻³ | 4.22 | nM | 319-331 |
| MD9 | 6.620 × 10⁴ | 1.462 × 10⁻³ | 22 | nM | 373-385 |
| *Tau12 | 1.030 × 10⁵ | 1.718 × 10⁻³ | 1.44 | nM | 6-18 |

*Commercially sourced Tau12 mAb binding kinetics and an affinity value was also measured and included in the table.

### Example 25: Antibodies Show Specificity to Human Tau in L66^{+/+} Brain Homogenates

Western blots with brain homogenates prepared from 3 animals of each genotype: wild-type (WT) mice; L66^{+/+} mouse; and L1^{+/+} mouse brain (all 5 months old) show specificity for human tau using CB7 (hT40 13-26) and CC7 (hT40 145-157) antibodies (Genotype and phenotype descriptions for L1 and L66^{+/+} are provided in the initial filing document). Protein equivalent to 20 μg from each brain extract was separated using 4-20% bis-tris gels and run in 1×MES buffer. FIG. 36 shows a blot stained with CB7 antibody. A clear band is visible in the lanes containing L66^{+/+} samples, but no other bands are detectable (WT or L1^{+/+}). FIG. 37 shows a similar outcome when a blot containing the same sample preparations is interrogated with the CC7 antibody. Once again, a band having relative mobility of 65 kDa is visible in the L66 sample with no other reactive bands present.

When these results are compared with western blots obtained with the core domain antibody binders S1D12 (hT40 337-355) (FIG. 38) and S1G2 (hT40 367-379) (FIG. 39) the results are very different with numerous bands visible in each mouse sample. A band is detected in each sample (WT, L66^{+/+} and L1^{+/+}) with an apparent molecular weight of approximately 55 kDa. This band most likely corresponds to endogenous mouse tau which is of a similar size. In the L66^{+/+} samples, a second band running at approximately 68 kDa is also detected. The presence of numerous other bands indicates proteolytic truncation of tau within brain homogenates. In addition, in FIG. 39 the S1G2 antibody is able to detect the pathologic 10-kDa tau fragment that is capable of seeding disease in other cells.

The amino acid sequences of the human and mouse tau regions that contain the epitopes of CB7, CC7, S1D12 and S1G2 are superimposed for comparison (FIG. 40). It is clear that both CB7 and CC7 epitopes fall in areas with no homology between the human and mouse tau sequences; however, in the core region, the sequences are identical. Together these blots and sequence analysis (FIGS. 36-40) highlight the diagnostic utility of these antibodies as they are able to recognise the presence of pathologic human tau in a transgenic mouse brain against a background of endogenous mouse tau protein. These antibodies could be utilised to track the fragmentation patterns of pathologic tau species during aging and in relation to any pharmacologic treatments that may affect the (human) tau protein, its aggregation, movement between compartments in the body (e.g. between brain and blood, between blood and saliva or between brain and blood and saliva) and its pattern of fragmentation.

### Example 26: Core and N-Terminal Antibody Pairings Detect Age-Related Tau Protein Truncations or Occlusion of Core Regions in Brain Homogenates of Ageing L66 Mice

Brains from L66^{+/+} mice of various ages (1.5-month, n=11; 3-month, n=11; and 5-month, n=8) were homogenised as previously described and protein content quantified by BCA assay. These brain homogenates were then screened in paired antibody ELISAs to assess age-related changes in the tau fragmentation pattern. Brain homogenates were included in a sandwich ELISA in duplicate and tau values were determined against the linear section of an hTau40 standard curve.

When using S1D12 antibody to capture tau from these brain homogenates and CB7 as the detector antibody, a marked decrease in tau signals was observed with increasing age (FIG. 41A). This finding was confirmed by changing the orientation of the assay and using CB7 as the capture antibody and S1G2 as detector (FIG. 41B). Since the pairings used in this assay detect tau fragments that contain the amino acids spanning from 13-379, any truncation or occlusion of epitopes as a result of pathology-related aggregation that masks the core will result in lower tau detection. The progressive loss of signal that is observed here suggests that either a truncation or epitope occlusion event is occurring or that multiple events of this nature are occurring during the aging of L66^{+/+} mice.

In order to gain further insights into the protein fragmentation state of the tau matrix in the samples investigated above, the levels of a smaller N′-terminal fragment were determined using the CB7 antibody capture paired with HT7, a commercial antibody with an epitope in the region tau159-163. Interestingly, there was a trend towards an increase in signal as the L66^{+/+} mice aged. So, as the longer core to N′-terminal tau fragment decreases with age (FIG. 41), the levels of smaller, truncated fragments of human tau increase in this transgenic mouse line (FIG. 42).

### Example 27: Ultrasensitive Assays for the Detection of Tau Fragments in Biological Fluids

Utilising Single Molecule Array (Simoa®) technology we have lowered the limit of detection of our assays substantially, in some instances to below 1 pg/ml tau protein (or protein fragment). Simoa® is a bead-based technology where a capture antibody is coated on to magnetic beads which can then be concentrated out of solution using a magnet. A biotinylated detector is added which binds to the captured molecule of interest. Streptavidin β-D-Galactosidase (SBG) binds to the detector and subsequently hydrolyses resorufin β-D-galactopyranoside (RPG) into a fluorescent product that is used for detection of the immunocomplex. Table 26 summarises the antibody pairings and limits of detection for assays successfully transferred to the Simoa® system.

TABLE 26
Assays that have successfully been transferred to Simoa ®.
Capture antibody is coated to magnetic beads and the detector antibody
is biotinylated. Limits of detection for each assay are included.

| | | | Simoa ® limit |
|---|---|---|---|
| | | | of detection |
| Assay | Capture antibody | Detector antibody | (pg/ml) |
| Core | S1D12 | S1G2 | 0.75 |
| (Tau 337-379) | (Binding region | (Binding region | |
| | hT40 337-355) | hT40 367-379) | |
| Core - proline | S1G2 | HT7 | 0.32 |
| (Tau 159-379) | | (Binding region | |
| | | hT40 159-163) | |
| Core - N′ | S1D12 | HT7 | 0.32 |
| (Tau 1-379) | S1D12 | BT2 | 0.32 |
| | | (Binding region | |
| | | hT40 194-198) | |
| | S1D12 | Tau12 | 0.75 |
| | | (Binding region | |
| | | hT40 6-18) | |
| | S1G2 | Tau12 | 1.6 |
| | S1G2 | 3bG4 | 20 |
| | | (Binding region | |
| | | hT40 1-15) | |
| Full-length | CB7 | Tau46 | 2.2 |
| (Tau 1-441) | (Binding region | (Binding region | |
| | hT40 13-25) | hT40 409-441) | |
| N′ - proline | CB7 | BT2 | 0.75 |
| (Tau 6-198) | CB7 | HT7 | 2.2 |
| | Tau12 | HT7 | 2.2 |
| Core - C′ | S1G2 | Tau46 | 2.2 |
| (Tau 337-441) | | | |

### Example 28: Measuring Tau Fragment Levels in AD and Healthy Control Plasma Samples

Human plasma samples were sourced from the commercial supplier Logical Biological Ltd. Healthy control (HC, n=12) and samples from individuals with a diagnosis of AD or mild cognitive impairment (AD/MCI, n=42) were aliquoted into low bind microfuge tubes and stored at −80° C.

The Simoa® experiments used for plasma screening utilised a 3-step protocol optimised for use with plasma samples. A standard curve (540 pg/ml−0.7 pg/ml) was created by spiking recombinant hTau40 into Tau 2.0 diluent and triple diluting. Plasma samples were diluted 1:100 and added in duplicate to the Simoa® plate followed by the addition of respective capture beads. Different antibody pairings used for plasma screening is explained in Table 28. In general, the capture antibody coated beads are mixed with diluted plasma samples, added to the plates and incubated for 30 min at 30° C. with shaking. Following the incubation, the plates were washed using the Simoa® plate washer before the addition of 0.2 μg/ml biotinylated detector antibody which was left to incubate as before for 10 min. The plates were then washed again before the addition of SBG and a final 10 min incubation. After the final incubation and wash steps the plates were transferred to the Simoa® reader and plasma tau concentrations generated against a 4-parameter fit curve.

TABLE 28
Table summarises the antibody pairings used for Simoa ® assays
for human plasma sample screening. The capture and detector
antibodies used in each assay are stated along with the tau
fragment each assay (the numbers refer to the amino acid
positions in the full-length human tau protein hT40).

| | | | Tau Protein Fragment |
|---|---|---|---|
| | | | Measured |
| | | | (numbers refer to |
| | Capture | Detector | amino acids in |
| Assay | Antibody | Antibody | the tau protein) |
| Core | S1D12 | S1G2 | 337-379 |
| NT1 | BT2 | Tau12 | 6-198 |
| Core-Tau12 | S1D12 | Tau12 | 6-349 |
| Core - HT7 | S1D12 | HT7 | 159-349 |
| Core - BT2 | S1D12 | BT2 | 194-359 |
| BT2 - HT7 | BT2 | HT7 | 159-198 |
| Core - C′ | S1D12 | Tau46 | 337-441 |
| BT2 - S1G2 | BT2 | 1G2 | 194-379 |

Human plasma screening data were subsequently analysed in Graphpad Prism v5. Interestingly, we have observed significantly higher levels of plasma tau fragments in age-matched, healthy control group compared to the AD group as detected using various capture-detector antibody pairings (Table 29).

TABLE 29
Results of Simoa ® experiments utilising 8 different pairings detecting tau fragments in human plasma samples. Assay
antibody pairings are as follows; NT1 (BT2-Tau12), Core (S1D12-S1G2), Core-Tau12 (S1D12-Tau12), Core-HT7 (S1D12-HT7), Core-
BT2 (S1D12-BT2), BT2-HT7 (BT2-HT7), Core-C' (S1D12-Tau46), BT2-S1G2 (BT2-S1G2). There is a significant decrease in core-
proline tau levels (measured using core-HT7 and core-BT2 assays where S1D12 is the capture antibody) in patients with an AD
diagnosis (AD/MCI) compared with health control (HC) plasma. The levels of tau measured when using our core capture antibody
S1D12 as part of an antibody pair is 1,000-fold more than the typical values seen for the existing NT1 assay in human plasma.
In contrast to the reported NT1 assay, where AD/MCI patients typically show higher assay values than healthy controls, for
assays using S1D12 capture this pattern is reversed with health control samples showing higher tau fragment values than AD/MCI patients.

| | | | | | INT1 | Core | Core -Tau12 | Core -HT7 | Core -BT2 | BT2 -HT7 | Core -C | BT2 -S1G2 |
|---|---|---|---|---|---|---|---|---|---|---|---|---|
| | | Average | Average | Average | (6-198) | (337-379) | (6-349) | (194-359) | (159-349) | (159-198) | (337-441) | (192-379) |
| Category | n | Age (yr) | Sex (M) | MMSE score | (pg/ml) | (pg/ml) | (pg/ml) | (pg/ml) | (pg/ml) | (pg/ml) | (pg/ml) | (pg/ml) |
| AD/MCI | 12 | 62 | 44.4% | 21 | 0.19 | n.d. | 0.67 | 30.24 | 899.6 | 44.51 | n.d. | n.d. |
| Healthy | 9 | 56 | 41.6% | NA | 0.145 | n.d. | 0.67 | 1,802 | 13,619 | 123.8 | n.d. | n.d. |
| Control | | | | | | | | | | | | |

| | | | | | | | | |
|---|---|---|---|---|---|---|---|---|
| Assay limit of detection (pg/ml) | 0.25 | 2.2 | 0.25 | 0.25 | 10.75 | 0.25 | 2.2 | 2.2 |

Data presented as mean.
n.d.—not detected (signals below blank)

### Example 29: Core Capture Antibody S1D12 can Measure Ng Levels of Tau in Human Plasma

When S1D12-BT2 or S1D12-HT7 pairings are used in Simoa® assay, nanogram levels of tau fragments are detected in human plasma (Table 29). All studies reported so far have measured pg/ml concentration of tau fragments in plasma with highest levels ˜850 pg/ml (Sparks et al 2012; Rani et al 2017). Interestingly, and contrary to the NT1 assay, this core-BT2 assay measures 1,000- to 10,000-fold more tau fragments and the levels are higher in healthy controls compared to AD patients (FIG. 43). Similarly, with the core-HT7 assay, ng/ml concentration of tau was detected in healthy control plasma and the levels were lower in AD/MCI plasma. Both findings translate to higher levels of core-proline fragments in healthy control and form the basis of a regular monitoring test to identify patients worthy of additional screening, i.e. a potential predictor of early onset of disease. The levels of tau detected using the core capture antibody is significantly greater than previously reported and suggests that the use of S1D12 reveals large amounts of previously undetected tau fragments in biological samples which can be considered as a tau protein mixture (full-length tau protein and populations of tau protein fragments) that can be referred to as a ‘tauosome’. The decrease in tau levels in the plasma of AD/MCI patients suggests that perhaps defective clearance of tau from the brain is a critical factor in the pathogenesis of AD.

### Example 30: FTD Sample Analysis Using Simoa®

Three frontotemporal dementia (FTD) plasma samples kindly donated by Re:Cognition Health (London) were screened using the S1D12/BT2 assay in a 3-step format. Samples were diluted in Tau 2.0 buffer and 100 μl was added to the wells of a 96-well assay plate in duplicate. Samples were run against a standard curve made using a threefold dilution of hTau40 from 540-0.74 pg/ml.

Paramagnetic beads (Quanterix #103612) were prepared and activated by the following method provided by the manufacturer. Beads (4.2×10⁸, approximately 150 μl of vortexed stock beads) were washed using the wash buffer and treated with bead conjugation buffer following manufacturer's instructions. 1-Ethyl-3-(3-dimethylaminopropyl) carbodiimide (EDC) (Thermo Scientific #A35391) was prepared in bead conjugation buffer and added to the beads to achieve a final concentration of 0.3 mg/ml. The beads were subsequently incubated for 30 min on a Thermomixer™ at 1,000 rpm to activate. Following washing as before, the activated beads were mixed with 300 μl of S1D12 antibody at a concentration of 0.2 mg/ml by vortexed for 10 s and subsequently incubating for 2 h at 1,000 rpm on a Thermomixer at 4° C.

25 μl of S1D12 capture bead solution containing approximately 500,000 beads was added to each well of the assay plate and incubated on the Simoa® shaker for 30 minutes at 30° C. and 800 rpm. Following this incubation, the plate was washed on the Simoa® SR-X washer and 100 μl of biotin-BT2 detector antibody (Thermofisher #MN1010B) at a concentration of 200 ng/ml was added to each well. The plate was subsequently transferred to the Simoa® plate shaker and incubated for 10 minutes at 800 rpm and 30° C. 100 μL of Streptavidin β-galactosidase (SBG, Quanterix #103397) diluted to 150 pM in Simoa® SBG diluent (Quanterix #100376) was added to each well, incubated for 10 min at 800 rpm at 30° C., washed and left on the magnet for 10 min to dry. The plate was then transferred to the Simoa® SR-X instrument for analysis. Fluorogenic resorufin β-d-galactopyranoside (RPG, Quanterix #103159) substrate resuspended for at least 30 min with shaking at 800 rpm and 30° C. was also added to the instrument for each assay along with the assay plate.

Tau levels were determined from a 4-parameter-fit standard curve, multiplied by the dilution factor and reported in Table 30. High levels of plasma tau (ng/ml that can be detected by S1D12-BT2 pairing) was seen in this group of FTD samples which is similar to our observation in AD samples and has not been reported previously.

TABLE 30
Plasma S1D12/BT2 tau values for 3
frontotemporal dementia patients

| Sample | S1D12/BT2 plasma tau (ng/ml) |
|---|---|
| FTD Sample 1 | 1.074 |
| FTD Sample 2 | 0.957 |
| FTD Sample 3 | 1.283 |

### Example 31: Human Plasma Analysis Using Chemiluminescent ELISA

Human plasma from individuals with an AD diagnosis and those from cognitively unimpaired individuals were sourced (Logical Biological, Kent UK), divided into 100 μl aliquots and stored at −80° C.

ELISA assays to detect tau fragments in these samples were carried out as follows. A black Nunc Maxisorp™ plate was immobilised with 2.5 μg/ml of S1D12 mAb (100 μl). The plate was incubated at 37° C. for 1 h, washed 3 times with 200 μl/well PBST (0.05% Tween20) and blocked with 2% (w/V) milk powder in PBS by incubating for 1 h at 37° C. Following washing as before, 100 μl/well human plasma samples (diluted 1:6, 1:10, or 1:20 in PBST) was added to the assay plate in duplicate. Simultaneously, a calibrator curve was generated by triple diluting hTau40 from 10 ng/ml to 14 pg/ml and the plate was incubated for 1 h at room temperature. The plate was washed 5 times in PBST and biotinylated BT2 detector (Thermofisher #MN1010B) diluted to 200 ng/ml in PBST was added (100 μl/well) and incubated for 1 h at room temperature. 100 μl of streptavidin poly-HRP (Thermofisher #21140) (1:10,000 in PBST) was added to the washed plate, incubated for 1 h at room temperature and developed using 50 μl SuperSignal ELISA Femto Substrate (Thermo Scientific, #37075). The plate was read for total luminescence using Clariostar Plus microplate reader (BMG Labtech).

Tau concentrations were derived from a 4-parameter fit curve and presented as the average across each dilution tested (FIG. 45). As with the Simoa®-based analysis, higher levels of plasma tau fragments were observed in age-matched, healthy control group compared to the AD group.

### Example 32: Ultrasensitive Core Antibodies are Required to Detect Core/Proline Tau Fragments in Human Plasma

Various 3-step Simoa® assays were set up as previously described using antibody pairs outlined in Table 1.4 to assess the effects of using a proline region antibody as the capturing molecule alongside a core antibody as a detector for the measurement of tau fragments in human plasma.

The use of S1D12 as a capture molecule with the proline binders BT2 and HT7 promotes the recovery of higher levels of tau than have previously been reported in human plasma. Furthermore, the levels of these fragments appear to decrease in Alzheimer's disease. With a small subset of patient samples this has now been replicated using S1G2 as a capturing molecule. Both high affinity core antibodies detect high levels of Core/Proline Tau that decrease in AD. Interestingly, when using CA4 (core antibody binding to the region corresponding to amino acids 355-367 of hT40) as capture and BT2 as the detector, high levels of Core/Proline tau is detected in cognitively unimpaired (CU) samples only. The functional lower limit of quantification of the assay is around 1 ng/ml due to the comparatively lower affinity of CA4 antibody (3 nM vs 120 pM for S1D12 and 170 pM for S1G2) which makes it difficult to detect any core/proline tau in AD samples (Table 31).

When attempts were made to reverse the format of these assays (i.e. using BT2 or HT7 as the capture molecules with S1D12 or S1G2 as detectors), no tau was detected in these plasma samples. This was despite the assays having excellent calibrator curve characteristics (FIG. 46) and using more concentrated plasma, 1:20 as opposed to the 1:50 dilution required for when core binders are used as the capture molecule.

TABLE 31
Plasma tau levels detected in 4 AD and 4 CU patients using various assays.

| | | Plasma Tau (pg/ml) | | | | | | | | |
|---|---|---|---|---|---|---|---|---|---|---|
| | | S1D12/ | S1D12/ | S1G2/ | S1G2/ | BT2/ | BT2/ | HT7/ | HT7/ | CA4/ |
| | Assay | BT2 | HT7 | BT2 | HT7 | S1D12 | S1G2 | S1D12 | S1G2 | BT2 |
| | Dilution | 1:50 | 1:50 | 1:50 | 1:50 | 1:20 | 1:20 | 1:20 | 1:20 | 1:6 |
| AD | PL 2222 | 716.6 | 355.8 | 113.0 | 57.3 | nd | nd | nd | nd | nd |
| | PL 2224 | 806.4 | 460.6 | 79.9 | 37.8 | nd | nd | nd | nd | nd |
| | PL 2226 | 843.9 | 389.4 | 96.7 | 61.0 | nd | nd | nd | nd | nd |
| | PL 2228 | 527.2 | 412.7 | 67.7 | 49.8 | nd | nd | nd | nd | nd |
| CU | PL 2457 | 6327.7 | 2714.9 | 1771.4 | 453.4 | nd | nd | nd | nd | 4963 |
| | PL 2458 | 23486.1 | 4987.3 | 4257.9 | 1184.8 | nd | nd | nd | nd | 12590.9 |
| | PL 2459 | 12543.6 | 2725.8 | 2712.2 | 657.2 | nd | nd | nd | nd | 4098.57 |
| | PL 2460 | 2225.3 | 903.6 | 705.8 | 207.2 | nd | nd | nd | nd | 1484.8 |

nd = not detected

### Example 33: Immunoprecipitation and Tryptic Digestion LC-MS Reveal Core Region Containing Tau Fragments in Human Plasma

To determine the identities of tau fragments detected using our antibody pairings, human plasma sample from a cognitively unimpaired (CU) individual was subjected to immunoprecipitation (IP) using S1D12 and BT2/HT7 antibodies followed by trypsin digestion and liquid chromatography-mass spectrometry analysis (LC-MS). A control sample was also included where 1×PBS was spiked with 50 ng hT40. Samples were immunoprecipitated with S1D12 antibody alone or double immunoprecipitated using S1D12 and BT2/HT7 antibodies. For immunoprecipitation, 10 μg of S1D12 antibody was covalently conjugated to 1.4×10⁷ magnetic beads and 2 ml of cognitively unimpaired human plasma or hT40 spiked sample was mixed with antibody coated beads for 1 h at room temperature. Using a magnetic separator, beads were washed with 1 ml of 0.1% PBST for 4 times and the tau fragments eluted using 30 μl of 0.1 M glycine (pH 3.0). After PH neutralisation with 6 μl of 1M Tris HCl (pH 8.0), the final volume was brought up to 1 ml by adding 1×PBS and magnetic beads conjugated with BT2/HT7 antibody (prepared as above) was added to the mixture (for double immunoprecipitation). Following 4 wash steps, the final elution was performed as previously described.

The samples were diluted using 50 mM ammonium bicarbonate and reduced with 200 mM dithiothreitol for 25 min at 60° C. Alkylation of cysteine residues was achieved by adding 200 mM iodoacetamide and incubation for 30 min at 25° C. in the dark. Trypsin digestion was performed by adding 40 μg sequencing grade trypsin (porcine, Promega V5111) and overnight incubation at 37° C. The sample was freeze dried by vacuum centrifugation (SpeedVac) and dissolved in 10 μl of 0.1% trifluoroacetic acid by mixing on an orbital shaker and centrifugation at 14,000 rpm for 2 min. Digested sample was analysed by injecting 9 μl of the solution on to a Q Exactive Plus LC-MS system. The liquid chromatography system UltiMate 3000 RSLCnano (Thermo Scientific Dionex) was configured for pre-concentration onto a nano column PepMap RSLC C18 75 μm i.d.×25 cm (Thermo Scientific P/N ES801). This was coupled with Q Exactive Plus hybrid quadrupole—Orbitrap mass spectrometer (Thermo Scientific) with EASY-Spray nano electrospray source. A 60-min liquid-chromatography (LC) gradient was applied with an increasing percentage of buffer B (acetonitrile:UHQ water:formic acid at a ratio of 80:20:0.1) for peptide elution; at the flow rate of 300 nL/min. Full MS1 scan was acquired from 375 to 1750 m/z in the Orbitrap at a resolution of 70,000, followed by the MS2 scan on the top 10 precursor ions at a resolution of 17,500 in a data-dependent acquisition (DDA) mode.

The raw files were processed using Proteome Discoverer version 2.2 (Thermo Scientific) and database search conducted with Mascot Server (version 2.6)

Shown in FIG. 47 are the tau fragments detected by abundance from the human plasma sample following immunoprecipitation and LC-MS analysis. Fragments detected from the spiked sample are also given for comparison.

### Example 34: S1D12 Immunotherapy-6-Week Repeat-Dose Mouse Study

A total of 12 wildtype, 12 Line 1 and 12 Line 66 female mice were used in this study—Line 1 and Line 66 are transgenic mouse models of tauopathy (Melis et al., 2015). Animals were 2 months old at the beginning of the study. Mice of each genotype were injected intraperitoneally (i.p.) with either vehicle or S1D12 mAb (30 mg/kg), once per week, for six consecutive weeks. This study design is summarised in Table 32 below

TABLE 32
Study design for S1D12 mAb 6-week repeat dosing in mice

| | | | | 6 × Weekly | |
|---|---|---|---|---|---|
| Number of | | | Dose | Doses | Dose |
| Mice | Genotype | Gender | Route | (S1D12) | Volume |
| 6 | NMRI | F | i.p | 30 mg/kg | 5 mL/kg |
| 6 | NMRI | F | i.p | Vehicle (PBS) | 5 mL/kg |
| 6 | Line 66 | F | i.p | 30 mg/kg | 5 mL/kg |
| 6 | Line 66 | F | i.p | Vehicle (PBS) | 5 mL/kg |
| 6 | Line 1 | F | i.p | 30 mg/kg | 5 mL/kg |
| 6 | Line 1 | F | i.p | Vehicle (PBS) | 5 mL/kg |

Seven days post final dose, cardiac puncture blood sampling was performed. mice were anaesthetised using an overdose of sodium pentobarbital (i.p.) and blood was collected via cardiac puncture through a Plastipak syringe pre-rinsed with heparinised saline (10 U/mL) and transferred into a plastic vial containing lithium heparin anticoagulant. Blood samples were centrifuged to obtain plasma at 2,000×g for 5 min at 6° C.

### Example 35: S1D12 Immunotherapy-Assessment of Plasma Tau Via Core-Core Simoa® Assay

In order to monitor the ‘treatment effect’ of S1D12 mAb, plasma tau of vehicle control and antibody treated L66^{+/+} mice were assessed via a core-core homebrew Simoa® assay to investigate changes in core tau pools in these mice. A three-step assay was performed and analysed by following the previously described plasma screening methods using CA4 antibody capture beads, S1G2 detector (10 ng/ml) and the plasma samples added in 1:8 dilution.

A highly significant increase in the level of plasma tau was detected in mice treated with S1D12 mAb when using a core and core antibody pairing (CA4-S1G2) in L1 mouse in the Simoa® assay (L1 with S1D12 treatment >100 ng/ml) vs L1 without S1D12 treatment (3.43 (0.45) ng/ml). This assay can detect both human and mouse tau and a statistically significant increase in the tau levels was observed in L66^{+/+} mice receiving the antibody treatment (L66^{+/+} with S1D12 treatment 3.52 (0.20) ng/ml) vs L66^{+/+} without S1D12 treatment (0.76 (0.21) ng/ml). To some extend a statistically significant increase in the tau levels was also seen in wildtype mice with SD12 treatment vs no treatment (3.57 (0.22) ng/ml) vs (1.91 (0.28) ng/ml) (FIG. 48). However, a profound increase was seen in L1 group, which was above the upper limit of detection of the current assay.

### Example 36: Measuring Tau Fragments in Saliva

Saliva samples were acquired from volunteers using the following protocol. Participants were instructed to fast for at least 30 min prior to giving the sample and, 10 min before sampling, were instructed to wash their mouths with water. Participants then produced at least 1 ml of unstimulated saliva into a 50 ml tube. 1 ml from each participant was transferred into a separate microfuge tube and centrifuged at 10,000×g for 5 min. The supernatant fraction was collected, aliquoted and stored at −80° C.

3-step Simoa® assays using various antibody pairings were run as described previously using assays described in Table 33 and the results summarised in Table 34.

TABLE 33
3-step Simoa ® assays used for saliva sample measurements

| | Beads (epitope) | | | | | |
|---|---|---|---|---|---|---|
| | S1D12 | S1D12 (337- | S1G2 | S1G2 | BT2 | BT2 |
| | (337-349) | 349) | (367-379) | (367-379) | (194-198) | (194-198) |
| | Detector (epitope) | | | | | |
| | BT2 | HT7 | BT2 | HT7 | S1D12 (337- | S1G2 |
| | (194-198) | (159-163) | (194-198) | (159-163) | 349) | (367-379) |
| Detector | 200 | 200 | 200 | 200 | 50 | 50 |
| concentration | | | | | | |
| (ng/ml) | | | | | | |
| Sample Dilution | 1:50 | 1:50 | 1:50 | 1:50 | 1:6 | 1:6 |

TABLE 34
Saliva tau values from various 3-Step Simoa ® assays.

| | | S1D12/BT2 | S1D12/HT7 | S1G2/BT2 | S1G2/HT7 | BT2/S1D12 | BT2/S1G2 |
|---|---|---|---|---|---|---|---|
| | | (pg/ml) | (pg/ml) | (pg/ml) | (pg/ml) | (pg/ml) | (pg/ml) |
| Sample | 1 | 186.0 | 32.6 | nd | 14.02 | nd | nd |
| | 2 | 75.6 | 43.4 | 22.9 | 63.84 | | |
| | 3 | 252.0 | 60.5 | 146.2 | 72.7 | | |
| | 4 | 277.9 | 91.0 | 585.8 | 61.4 | | |
| | 5 | 84.5 | 25.5 | 37.48 | 49.1 | | |
| | 6 | 21.2 | nd | 65.2 | 119.6 | | |
| | 7 | 22242.7 | 98623.6 | 6271.1 | 147.33 | | |
| | 8 | 14.8 | nd | 53.6 | 54.74 | | |
| | 9 | 367.2 | 81.2 | 192 | 27.94 | | |
| | 10 | 52.2 | nd | 27.06 | 37.09 | | |
| | 11 | 242.3 | 37.6 | 134.98 | 52.42 | | |

nd = not detected

Surprisingly, reversal of the antibody pairings to use BT2 as the capture antibody and either S1D12 or S1G2 as the detector antibody, abolished detection. This contrasts with the data presented in Example 26 and FIG. 41, where core and N-terminal pairings detected tau in brain homogenate samples irrespective of whether S1D12/S1G2 (core binders) and CB7 (which binds to hT40 13-26) were used as the capture or the detector antibody. This suggests that using a capture antibody that binds to an epitope within residues 297 to 391 of SEQ ID NO: 1 is advantageous in saliva samples.

1. An in vitro method for detecting a tau protein or fragment thereof in a saliva sample comprising contacting the sample with a first specific binding molecule wherein the first specific binding molecule binds to an epitope within residues 297 to 391 of SEQ ID NO: 1.

2. The method of claim 1, wherein the first specific binding molecule binds to an epitope within residues 307 to 391 of SEQ ID NO: 1; 337 to 379 of SEQ ID NO: 1; 337 to 349 of SEQ ID NO: 1; or 337 to 355 of SEQ ID NO: 1.

3-5. (canceled)

6. The method of claim 2, wherein the first specific binding molecule comprises the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 16 (NNAVG);
VHCDR2 comprises the sequence set forth in SEQ ID NO: 18 (GCSSDGTCYYNSALKS);
VHCDR3 comprises the sequence set forth in SEQ ID NO: 21 (GHYSIYGYDYLGTIDY);
VLCDR1 comprises the sequence set forth in SEQ ID NO: 24 (SGSSSNVGGGNSVG);
VLCDR2 comprises the sequence set forth in SEQ ID NO: 26 (DTNSRPS);
VLCDR3 comprises the sequence set forth in SEQ ID NO: 29 (VTGDSTTHDDL);
or for each CDR sequence, an amino acid sequence with
(i) at least 85% identity thereto, and/or
(ii) one, two, or three amino acid substitutions relative thereto.

7. The method of claim 6, wherein the first specific binding molecule comprises the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 16 (NNAVG);
VHCDR2 comprises the sequence set forth in SEQ ID NO: 18 (GCSSDGTCYYNSALKS);
VHCDR3 comprises the sequence set forth in SEQ ID NO: 21 (GHYSIYGYDYLGTIDY);
VLCDR1 comprises the sequence set forth in SEQ ID NO: 24 (SGSSSNVGGGNSVG);
VLCDR2 comprises the sequence set forth in SEQ ID NO: 26 (DTNSRPS); and
VLCDR3 comprises the sequence set forth in SEQ ID NO: 29 (VTGDSTTHDDL).

8-9. (canceled)

10. The method of claim 6, wherein the first specific binding molecule specifically binds to a polypeptide or protein molecule comprising an amino acid sequence comprising residues 337 to 355 of SEQ ID NO: 1 with a K_{D} of less than around 500 pM.

11. (canceled)

12. The method of claim 1, wherein the first specific binding molecule comprises the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 54 (SYRADGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS);
VLCDR3 comprises the sequence set forth in SEQ ID NO: 73 (GSSDRTPYTGV);
or for each CDR sequence, an amino acid sequence with
(i) at least 85% identity thereto, and/or
(ii) one, two, or three amino acid substitutions relative thereto.

13. The method of claim 12, wherein the first specific binding molecule comprises the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 42 (SNSVG);
VHCDR2 comprises the sequence set forth in SEQ ID NO: 46 (GIDTDGEEGYNPALNS);
VHCDR3 comprises the sequence set forth in SEQ ID NO: 54 (SYRADGLAYGYVQAIDY);
VLCDR1 comprises the sequence set forth in SEQ ID NO: 63 (SGSFIGISSVG);
VLCDR2 comprises the sequence set forth in SEQ ID NO: 70 (ASDGRPS); and
VLCDR3 comprises the sequence set forth in SEQ ID NO: 73 (GSSDRTPYTGV).

14-15. (canceled)

16. The method of claim 14, wherein the first specific binding molecule specifically binds to a polypeptide or protein molecule comprising an amino acid sequence comprising residues 367 to 379 of SEQ ID NO: 1 with a K_{D} of less than around 500 pM.

17. (canceled)

18. The method of claim 1, wherein the first specific binding molecule comprises the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 83 (SYSVY);
VHCDR2 comprises the sequence set forth in SEQ ID NO: 84 (IMYASGRVDYNPALKS);
VHCDR3 comprises the sequence set forth in SEQ ID NO: 89 (GIEN);
VLCDR1 comprises the sequence set forth in SEQ ID NO: 91 (RTSQSVNNYLS);
VLCDR2 comprises the sequence set forth in SEQ ID NO: 95 (YATRLYT); and
VLCDR3 comprises the sequence set forth in SEQ ID NO: 97 (LQYDSTPLA);
or for each CDR sequence, an amino acid sequence with
(i) at least 85% identity thereto, and/or
(ii) one, two, or three amino acid substitutions relative thereto.

19. The method of claim 18, wherein the first specific binding molecule comprises the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 83 (SYSVY);
VHCDR2 comprises the sequence set forth in SEQ ID NO: 84 (IMYASGRVDYNPALKS);
VHCDR3 comprises the sequence set forth in SEQ ID NO: 89 (GIEN);
VLCDR1 comprises the sequence set forth in SEQ ID NO: 91 (RTSQSVNNYLS);
VLCDR2 comprises the sequence set forth in SEQ ID NO: 95 (YATRLYT); and
VLCDR3 comprises the sequence set forth in SEQ ID NO: 97 (LQYDSTPLA).

20-22. (canceled)

23. The method of claim 1, further comprising contacting the sample with a second specific binding molecule.

24. The method of claim 23 wherein the second specific binding molecule binds to an epitope within residues 151 to 243 of SEQ ID NO: 1; 194 to 198 of SEQ ID NO: 1; or 159 to 163 of SEQ ID NO: 1.

25. (canceled)

26. The method of claim 24, wherein the second specific binding molecule is BT2 or HT7.

27-29. (canceled)

30. The method of claim 26, wherein the first specific binding molecule is S1D12 or S1G2 and the second specific binding molecule is BT2 or HT7.

31. The method of claim 23, wherein the second specific binding molecule comprises the CDRs VHCDR1, VHCDR2, VHCDR3, VLCDR1, VLCDR2 and VLCDR3, wherein each of said CDRs comprises an amino acid sequence as follows:
VHCDR1 comprises the sequence set forth in SEQ ID NO: 198 (SNAVI);
VHCDR2 comprises the sequence set forth in SEQ ID NO: 200 (LIDVDGDAAYDPALKS);
VHCDR3 comprises the sequence set forth in SEQ ID NO: 202 (DYGSWGYVSDIDY);
VLCDR1 comprises the sequence set forth in SEQ ID NO: 204 (SGSDIGGADVG);
VLCDR2 comprises the sequence set forth in SEQ ID NO: 206 (DNDNRPS); and
VLCDR3 comprises the sequence set forth in SEQ ID NO: 208 (GTYSGANYGI);
or for each CDR sequence, an amino acid sequence with
(i) at least 85% identity thereto, and/or
(ii) one, two, or three amino acid substitutions relative thereto,
wherein the specific binding molecule binds to a polypeptide or protein molecule comprising an amino acid sequence comprising residues 147 to 157 of SEQ ID NO: 1.

32. The method of claim 23, wherein the second specific binding molecule comprises the CDRs VHCDR1, VHCDR2, VHCDR3, VLCDR1, VLCDR2 and VLCDR3,
VHCDR1 comprises the sequence set forth in SEQ ID NO: 17 (SNAVG);
VHCDR2 comprises the sequence set forth in SEQ ID NO: 201 (LIDIDGDTAYNPALES);
VHCDR3 comprises the sequence set forth in SEQ ID NO: 203 (HYDKWGYADSIDY);
VLCDR1 comprises the sequence set forth in SEQ ID NO: 138 (SGSSSNVGYGDYVG);
VLCDR2 comprises the sequence set forth in SEQ ID NO: 207 (DATTRAS); and
VLCDR3 comprises the sequence set forth in SEQ ID NO: 209 (ASYQNERSGV);
or for each CDR sequence, an amino acid sequence with
(i) at least 85% identity thereto, and/or
(ii) one, two, or three amino acid substitutions relative thereto,
wherein the specific binding molecule binds to a polypeptide or protein molecule comprising an amino acid sequence comprising residues 147 to 157 of SEQ ID NO: 1.

33. The method of claim 23, wherein the second specific binding molecule comprises the CDRs VHCDR1, VHCDR2, VHCDR3, VLCDR1, VLCDR2 and VLCDR3, wherein each of said CDRs comprises an amino acid sequence as follows:
VHCDR1 comprises a VHCDR1 amino acid sequence set forth in table 10;
VHCDR2 comprises a VHCDR2 amino acid sequence set forth in table 10;
VHCDR3 comprises a VHCDR3 amino acid sequence set forth in table 10;
VLCDR1 comprises a VLCDR1 amino acid sequence set forth in table 10;
VLCDR2 comprises a VLCDR2 amino acid sequence set forth in table 10; and
VLCDR3 comprises a VLCDR3 amino acid sequence set forth in table 10;
or for each CDR sequence, an amino acid sequence with
(i) at least 85% identity thereto, and/or
(ii) one, two, or three amino acid substitutions relative thereto,
wherein the specific binding molecule binds to an epitope within SEQ ID NO: 1.

34. The method of claim 33, wherein the second specific binding molecule comprises the CDRs of a clone selected from the group consisting of 3bD11, CB11, CA2, CB6, CA7, CA8, CB10, CC7, CB12, CC3, CA1, CA3, CD2, CC4, CD1 and CC5.

35. The method of claim 23, wherein the first and/or second specific binding molecule is an immunoglobin, an immunoglobin Fab region, a Fab′, a Fv, a Fv-Fc, a single chain Fv (scFv), scFv-Fc, (scFv) 2, a diabody, a triabody, a tetrabody, a bispecific t-cell engager (BiTE), an intein, a VNAR domain, a single domain antibody (sdAb) or a VH domain.

36-53. (canceled)


SEQUENCE LISTING (636 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 97 aa, Homo sapiens
DNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAE

SEQ ID NO: 4 - protein, 95 aa, Homo sapiens
IKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAE

SEQ ID NO: 5 - protein, 94 aa, Homo sapiens
IKHVPGGGSVQIVYKPVDLSKVTSKCGSLGNIHHKPGGGQVEVKSEKLDFKDRVQSKIGSLDNITHVPGGGNKKIETHKLTFRENAKAKTDHGA

SEQ ID NO: 6 - protein, 73 aa, Homo sapiens
VQIVYKPVDLSKVTSKCGSLGNIHHKPGGGQVEVKSEKLDFKDRVQSKIGSLDNITHVPGGGNKKIETHKLTF

SEQ ID NO: 7 - protein, 71 aa, Homo sapiens
IVYKPVDLSKVTSKCGSLGNIHHKPGGGQVEVKSEKLDFKDRVQSKIGSLDNITHVPGGGNKKIETHKLTF

SEQ ID NO: 8 - protein, 13 aa, synthetic construct
  REGION 1..13: note = S1D12 epitope
VEVKSEKLDFKDR

SEQ ID NO: 9 - skipped

SEQ ID NO: 10 - protein, 5 aa, synthetic construct
  REGION 1..5: note = 337-355 VHCDR1 consensus
  VARIANT 1: note = N or S
XNAVG

SEQ ID NO: 11 - protein, 16 aa, synthetic construct
  REGION 1..16: note = 337-355 VHCDR2 consensus
  VARIANT 10: note = Y or H
  VARIANT 7: note = T or K
GCSSDGXCYXNSALKS

SEQ ID NO: 12 - protein, 16 aa, synthetic construct
  REGION 1..16: note = 337-355 VHCDR3 consensus
  VARIANT 4: note = S or P
  VARIANT 2: note = H, F or Y
  VARIANT 5: note = I or V
  VARIANT 11: note = L or S
GXYXXYGYDYXGTIDY

SEQ ID NO: 13 - protein, 14 aa, synthetic construct
  REGION 1..14: note = 337-355 VLCDR1 consensus
  VARIANT 10: note = G or R
  VARIANT 12: note = S or D
  VARIANT 14: note = G or A
  VARIANT 8: note = G or nothing
SGSSSNVXGXNXVX

SEQ ID NO: 14 - protein, 7 aa, synthetic construct
  REGION 1..7: note = 337-355 VLCDR2 consensus
  VARIANT 3: note = N or T
  VARIANT 1: note = D, N or G
XTXSRPS

SEQ ID NO: 15 - protein, 11 aa, synthetic construct
  REGION 1..11: note = 337-355 VLCDR3 consensus
  VARIANT 9: note = D or N
  VARIANT 2: note = T or S
  VARIANT 6: note = T or S
  VARIANT 1: note = V or A
  VARIANT 7: note = T or A
  VARIANT 11: note = L or I
  VARIANT 8: note = H or I
XXGDSXXXXDX

SEQ ID NO: 16 - protein, 5 aa, synthetic construct
  REGION 1..5: note = VHCDR1 S1D12, S2C1
NNAVG

SEQ ID NO: 17 - protein, 5 aa, synthetic construct
  REGION 1..5: note = VHCDR1 ME12, NS3D9; VHCDR1 S1D2, S1H6, S2C3; VHCDR1 MC5, MD12; VHCDR1 3aA6, VHCDR1 CE3
SNAVG

SEQ ID NO: 18 - protein, 16 aa, synthetic construct
  REGION 1..16: note = VHCDR2 S1D12, S2C1
GCSSDGTCYYNSALKS

SEQ ID NO: 19 - protein, 16 aa, synthetic construct
  REGION 1..16: note = VHCDR2 ME12
GCSSDGKCYHNSALKS

SEQ ID NO: 20 - protein, 16 aa, synthetic construct
  REGION 1..16: note = VHCDR2 NS3D9, CE3
GCSSDGKCYYNSALKS

SEQ ID NO: 21 - protein, 16 aa, synthetic construct
  REGION 1..16: note = VHCDR3 S1D12
GHYSIYGYDYLGTIDY

SEQ ID NO: 22 - protein, 16 aa, synthetic construct
  REGION 1..16: note = VHCDR3 ME12
GFYSIYGYDYSGTIDY

SEQ ID NO: 23 - protein, 16 aa, synthetic construct
  REGION 1..16: note = VHCDR3 NS3D9, CE3
GYYPVYGYDYLGTIDY

SEQ ID NO: 24 - protein, 14 aa, synthetic construct
  REGION 1..14: note = VLCDR1 S1D12, ME12
SGSSSNVGGGNSVG

SEQ ID NO: 25 - protein, 13 aa, synthetic construct
  REGION 1..13: note = VLCDR1 NS3D9, CE3
SGSSSNVGRNDVA

SEQ ID NO: 26 - protein, 7 aa, synthetic construct
  REGION 1..7: note = VLCDR2 S1D12
DTNSRPS

SEQ ID NO: 27 - protein, 7 aa, synthetic construct
  REGION 1..7: note = VLCDR2 ME12
NTNSRPS

SEQ ID NO: 28 - protein, 7 aa, synthetic construct
  REGION 1..7: note = VLCDR2 NS3D9, CE3
GTTSRPS

SEQ ID NO: 29 - protein, 11 aa, synthetic construct
  REGION 1..11: note = VLCDR3 S1D12
VTGDSTTHDDL

SEQ ID NO: 30 - protein, 11 aa, synthetic construct
  REGION 1..11: note = VLCDR3 ME12
VTGDSSTHDDL

SEQ ID NO: 31 - protein, 11 aa, synthetic construct
  REGION 1..11: note = VLCDR3 NS3D9, CE3
ASGDSSAINDI

SEQ ID NO: 32 - protein, 251 aa, synthetic construct
  REGION 1..251: note = S1D12 amino acid sequence
QVQLQESGPSLVKPSQTLSLTCTVSGFSLNNNAVGWVRQAPGKVPESLVGCSSDGTCYYNSALKSRLDITRDTSKNQISLSLSSVTTDDAAVYYCTRGHYSIYGYDYLGTIDYWGPGLLVTVSSEGKSSGASGESKVDDQAVLTQPSSVSGSLGQRVSITCSGSSSNVGGGNSVGWYQHLPGSGLKTIIYDTNSRPSGVPDRFSGSRSGNTATLTINSLQAEDEGDYYCVTGDSTTHDDLVGSGTRLTVLG

SEQ ID NO: 33 - protein, 13 aa, Homo sapiens
GNKKIETHKLTFR

SEQ ID NO: 34-35 - skipped (2 entries)

SEQ ID NO: 36 - protein, 16 aa, synthetic construct
  REGION 1..16: note = 367 to 379 VHCDR2 consensus
  VARIANT 10: note = Y or F
  VARIANT 15: note = N or K
  VARIANT 13: note = A or V
  VARIANT 7: note = E, Y, D or R
  VARIANT 1: note = G, S or N
  VARIANT 2: note = I or V
  VARIANT 4: note = T or S
  VARIANT 9: note = G, Y or F
  VARIANT 3: note = D or Y
  VARIANT 5: note = D or T
  VARIANT 8: note = E, T or A
XXXXXGXXXXNPXLXS

SEQ ID NO: 37 - skipped

SEQ ID NO: 38 - protein, 11 aa, synthetic construct
  REGION 1..11: note = 367 to 379 VLCDR1 consensus
  VARIANT 7: note = I, S or R
  VARIANT 5: note = I, L or V
  VARIANT 4: note = F, Y or D
  VARIANT 3: note = S or R
  VARIANT 9: note = S, R, A or G
  VARIANT 6: note = G or S
SGXXXXXSXVG

SEQ ID NO: 39 - protein, 14 aa, synthetic construct
  REGION 1..14: note = CC5, CA12, CA7, 412E10 and 412G11 VLCDR1
SGSSSNVGYGNYVG

SEQ ID NO: 40-41 - skipped (2 entries)

SEQ ID NO: 42 - protein, 5 aa, synthetic construct
  REGION 1..5: note = VHCDR1 S1G2, S1B1, S1D9, S1F4, S1G10, S1H9, S2C6, S2D1, S2D4, CA9, CC12, S1A12, S1A5, S1E12, CB9
SNSVG

SEQ ID NO: 43 - protein, 729 aa, synthetic construct
  REGION 1..729: note = E2B7 nucleotide sequence
CAGGTGCAGCTGCAGGAGTCGGGACCCAGCCTGGTGAAGCCCTCACAGACCCTCTCCCTCACCTGCACGGTCTCTGGATTCTCATTAACCAGCTGGGGTGTAGCTTGGGTCCGCCAGGCTCCAGGAAAGGCACTGGAGTGGCTTGGTACCATGCGTAGTGGTGGTGGTACAGAATATAATCCGGCCCTGAAATCCCGCCTCAGCATCACCAGGGACACCTCCAAGAGCCAAGTCTCCCTGTCACTGAGCAGCGTGACAACTGAGGACATGGCCATGTACTACTGTGCCAGAGGTTATTTGAGTGGTATTCATTATGCCTGGGGCCGAGGACTCCTAGTCTCCGTCTCCTCAGAAGGTAAATCTTCTGGCGCGTCTGGCGAGTCTAAAGTGGATGACCAGGCTGTGCTGACTCAGCTGTCCTCCGTGTCTGGGTCCCTGGGCCAGAGGGTCTCCATCACCTGCTCTGGAAGCAGCAGCAACGTTGGAGATGGTGATTATGTGGGCTGGTTCCAACAGCTCCCAGGATCAGCCCCCAAACTCCTCATCTATAGTGCGCGCAATCGAGCCTCGGGGGTCCCCGACCGATTCTCCGGCTCCAGGTCTGGCAACACAGCGACTCTAACCATCACCTCGCTCCAGGCTGAGGACGAGGCCGATTATTACTGTGCATCTATTGACACCAGTCGCTCTCACATTTTCGGCAGCGGGACCAGACTGACCGTCCTGGGT

SEQ ID NO: 44 - protein, 5 aa, synthetic construct
  REGION 1..5: note = VHCDR1 CA12, VHCDR1 CA7
SYYVG

SEQ ID NO: 45 - protein, 5 aa, synthetic construct
  REGION 1..5: note = VHCDR1 CB2, NS2A1
TNSVG

SEQ ID NO: 46 - protein, 16 aa, synthetic construct
  REGION 1..16: note = VHCDR2 S1G2, S1B1, S1F4, S1G10, S2D1, S2D4, CA9, S1A12, S1A5, S1E12
GIDTDGEEGYNPALNS

SEQ ID NO: 47 - protein, 16 aa, synthetic construct
  REGION 1..16: note = VHCDR2 S1D2, S1H6, MD12
SVDSDGYTYYNPALKS

SEQ ID NO: 48 - protein, 16 aa, synthetic construct
  REGION 1..16: note = VHCDR2 S1D9, CC12, S1D5, CB9
GIDSDGEEGYNPALNS

SEQ ID NO: 49 - protein, 16 aa, synthetic construct
  REGION 1..16: note = VHCDR2 S1H9
GIDSDGEEGYNPALKS

SEQ ID NO: 50 - protein, 16 aa, synthetic construct
  REGION 1..16: note = VHCDR2 S2C3, MC5
SVDSDGDTYYNPALKS

SEQ ID NO: 51 - protein, 16 aa, synthetic construct
  REGION 1..16: note = VHCDR2 S2C6
GIDTDGEEGYNPALKS

SEQ ID NO: 52 - protein, 16 aa, synthetic construct
  REGION 1..16: note = VHCDR2 CA12, VHCDR2 CA7
NIYSTGRAFYNPALKS

SEQ ID NO: 53 - protein, 16 aa, synthetic construct
  REGION 1..16: note = VHCDR2 CB2, NS2A1
GIDTDGEEGFNPVLKS

SEQ ID NO: 54 - protein, 17 aa, synthetic construct
  REGION 1..17: note = VHCDR3 S1F4, S1G2, S1G10, CC12, S1E12, S1D5, CB9
SYRADGLAYGYVQAIDY

SEQ ID NO: 55 - protein, 17 aa, synthetic construct
  REGION 1..17: note = VHCDR3 S1B1, S2D1, CB2, NS2A1
SYRTDGLAYGYVQAIDY

SEQ ID NO: 56 - protein, 13 aa, synthetic construct
  REGION 1..13: note = VHCDR3 S1D2, S1H6, MC5, MD12
SVNGHPDVYYIDR

SEQ ID NO: 57 - protein, 17 aa, synthetic construct
  REGION 1..17: note = VHCDR3 S1D9
TYRTDGYAYGYVQAIDY

SEQ ID NO: 58 - protein, 17 aa, synthetic construct
  REGION 1..17: note = VHCDR3 S1H9, CA9
SYRSDGLAYGYVQAIDY

SEQ ID NO: 59 - protein, 13 aa, synthetic construct
  REGION 1..13: note = VHCDR3 S2C3
SANGHPDVYYIDK

SEQ ID NO: 60 - protein, 17 aa, synthetic construct
  REGION 1..17: note = VHCDR3 S2C6
TYRTDGFAYGYVQAIDY

SEQ ID NO: 61 - protein, 17 aa, synthetic construct
  REGION 1..17: note = VHCDR3 S2D4
SYRTDGLAYGYVQAIEY

SEQ ID NO: 62 - protein, 124 aa, synthetic construct
  REGION 1..124: note = S1D12 antibody variable heavy chain
QVQLQESGPSLVKPSQTLSLTCTVSGFSLNNNAVGWVRQAPGKVPESLVGCSSDGTCYYNSALKSRLDITRDTSKNQISLSLSSVTTDDAAVYYCTRGHYSIYGYDYLGTIDYWGPGLLVTVSS

SEQ ID NO: 63 - protein, 11 aa, synthetic construct
  REGION 1..11: note = VLCDR1 S1G2, S1B1, S1D9, S1F4, S1G10, S1H9, CA9, S1A12, S1D5, S1E12, S2C6
SGSFIGISSVG

SEQ ID NO: 64 - protein, 11 aa, synthetic construct
  REGION 1..11: note = VLCDR1 S1D2, S2C3, MD12
SGSYISSSRVG

SEQ ID NO: 65 - protein, 11 aa, synthetic construct
  REGION 1..11: note = VLCDR1 S1H6
SGSDLGSSRVG

SEQ ID NO: 66 - protein, 11 aa, synthetic construct
  REGION 1..11: note = VLCDR1 S2D1
SGSYIGSSAVG

SEQ ID NO: 67 - protein, 11 aa, synthetic construct
  REGION 1..11: note = VLCDR1 S2D4, CC12, CB9
SGRFIGISSVG

SEQ ID NO: 68 - protein, 11 aa, synthetic construct
  REGION 1..11: note = VLCDR1 CB2, NS2A1
SGSYIGSSGVG

SEQ ID NO: 69 - protein, 11 aa, synthetic construct
  REGION 1..11: note = VLCDR1 MC5
SGSYVSRSRVG

SEQ ID NO: 70 - protein, 7 aa, synthetic construct
  REGION 1..7: note = 297-390 VLCDR2 alternative consensus sequence, VLCDR2 S1G2, S1B1, S1D9, S1F4, S1G10, S1H9, S2D1, S2D4, CA9, CB2, CC12, NS2A1, S1A12, S1D5, S1E12, CB9, S2C6
ASDGRPS

SEQ ID NO: 71 - protein, 7 aa, synthetic construct
  REGION 1..7: note = VLCDR2 S1D2, S1H6, S2C3, MC5, MD12
DSSSRPS

SEQ ID NO: 72 - protein, 7 aa, synthetic construct
  REGION 1..7: note = VLCDR2 CA12 and CA7
AATSRAS

SEQ ID NO: 73 - protein, 11 aa, synthetic construct
  REGION 1..11: note = VLCDR3 S1G2
GSSDRTPYTGV

SEQ ID NO: 74 - protein, 11 aa, synthetic construct
  REGION 1..11: note = VLCDR3 S1B1, S1D9, S1F4, S1G10, S1H9, CA9, CC12, S1A12, S1D5, S1E12, CB9, S2C6
GSSDRTQYTGV

SEQ ID NO: 75 - protein, 9 aa, synthetic construct
  REGION 1..9: note = VLCDR3 S1D2, MD12
GVFGDRNYI

SEQ ID NO: 76 - protein, 9 aa, synthetic construct
  REGION 1..9: note = VLCDR3 S1H6, S2C3
GIFGDRNYI

SEQ ID NO: 77 - protein, 11 aa, synthetic construct
  REGION 1..11: note = VLCDR3 S2D4
GSTAPTPHTGV

SEQ ID NO: 78 - protein, 10 aa, synthetic construct
  REGION 1..10: note = VLCDR3 CA12 and CA7
SSYQRGNTGV

SEQ ID NO: 79 - protein, 11 aa, synthetic construct
  REGION 1..11: note = VLCDR3 CB2 and NS2A1
GSSDRTQYTGL

SEQ ID NO: 80 - protein, 9 aa, synthetic construct
  REGION 1..9: note = VLCDR3 MC5
GIYGDRNYI

SEQ ID NO: 81 - protein, 249 aa, synthetic construct
  REGION 1..249: note = S1G2 amino acid sequence
QVQLQESGPSLVKPSQTLSLTCTVSGFSLTSNSVGWVRQAPGKAPEWVAGIDTDGEEGYNPALNSRLSITRDTSKSQVSLSLSSVTSEDTAVYYCGRSYRADGLAYGYVQAIDYWGPGLLVTVSSEGKSSGASGESKVDDQAVVTQPSSVSGSLGQRVSITCSGSFIGISSVGWFQQLPGSGLRTIIVASDGRPSGVPDRFSMSKSGNTATLTISSLQAEDEADYFCGSSDRTPYTGVFGSGTRLTVLG

SEQ ID NO: 82 - protein, 32 aa, Homo sapiens
VEVKSEKLDFKDRVQSKIGSLDNITHVPGGGN

SEQ ID NO: 83 - protein, 5 aa, synthetic construct
  REGION 1..5: note = 337 to 363 VHCDR1 consensus, VHCDR1 NS2A3, NS2A8, NS2C5, NS2C8, NS2D3, CA4
SYSVY

SEQ ID NO: 84 - protein, 16 aa, synthetic construct
  REGION 1..16: note = 337 to 363 VHCDR2 consensus, VHCDR2 NS2A3, NS2A8, NS2C5, NS2C8, NS2D3, CA4
IMYASGRVDYNPALKS

SEQ ID NO: 85-86 - skipped (2 entries)

SEQ ID NO: 87 - protein, 7 aa, synthetic construct
  REGION 1..7: note = 337 to 363 VLCDR2 consensus
  VARIANT 6: note = Y or H
YATYLXT

SEQ ID NO: 88 - protein, 9 aa, synthetic construct
  REGION 1..9: note = 337 to 363 VLCDR3 consensus
  VARIANT 4: note = D, G or E
  VARIANT 9: note = A or T
  VARIANT 5: note = S or T
LQYXXTPLX

SEQ ID NO: 89 - protein, 4 aa, synthetic construct
  REGION 1..4: note = VHCDR3 NS2A3, NS2A8, NS2C8, CA4
GIEN

SEQ ID NO: 90 - protein, 4 aa, synthetic construct
  REGION 1..4: note = VHCDR3 NS2C5, NS2D3
GIED

SEQ ID NO: 91 - protein, 11 aa, synthetic construct
  REGION 1..11: note = VLCDR1 NS2A3, NS2C8, CA4
RTSQSVNNYLS

SEQ ID NO: 92 - protein, 11 aa, synthetic construct
  REGION 1..11: note = VLCDR1 NS2A8
RTNESVGNYLS

SEQ ID NO: 93 - protein, 11 aa, synthetic construct
  REGION 1..11: note = VLCDR1 NS2C5
RTSQNIDNGLA

SEQ ID NO: 94 - protein, 11 aa, synthetic construct
  REGION 1..11: note = VLCDR1 NS2D3
RTSQSVGSYLS

SEQ ID NO: 95 - protein, 7 aa, synthetic construct
  REGION 1..7: note = VLCDR2 NS2A3, NS2C8, CA4
YATRLYT

SEQ ID NO: 96 - protein, 7 aa, synthetic construct
  REGION 1..7: note = VLCDR2 NS2A8, NS2C5, NS2D3
YATRLHT

SEQ ID NO: 97 - protein, 9 aa, synthetic construct
  REGION 1..9: note = VLCDR3 NS2A3, NS2C8, CA4
LQYDSTPLA

SEQ ID NO: 98 - protein, 9 aa, synthetic construct
  REGION 1..9: note = VLDDR3 NS2D3
LQYDSTPLT

SEQ ID NO: 99 - protein, 9 aa, synthetic construct
  REGION 1..9: note = VLCDR3 NS2C5
LQYESTPLA

SEQ ID NO: 100 - protein, 9 aa, synthetic construct
  REGION 1..9: note = VLCDR3 NS2A8
LQYGTTPLA

SEQ ID NO: 101 - protein, 22 aa, Homo sapiens
KKIETHKLTFRENAKAKTDHGA

SEQ ID NO: 102 - protein, 5 aa, synthetic construct
  REGION 1..5: note = 369 to 390 VHCDR1 consensus, NS4E3, NS3E5, NS3H4, NS4F2, NS2B6, MD9
RESIA

SEQ ID NO: 103 - protein, 16 aa, synthetic construct
  REGION 1..16: note = 369 to 390 VHCDR2 consensus, NS4E3, NS3E5, NS3H4, NS4F2, NS2B6, MD9
GVGIDGTSYYSPALKS

SEQ ID NO: 104 - protein, 7 aa, synthetic construct
  REGION 1..7: note = 369 to 390 VHCDR3 consensus, VHCDR3 NS4E3, NS3E5, NS3H4, NS4F2, NS2B6, MD9
NYIDFEY

SEQ ID NO: 105 - protein, 14 aa, synthetic construct
  REGION 1..14: note = 369 to 390 VLCDR1 consensus
  VARIANT 12: note = Y, G or D
  VARIANT 8: note = G or I
  VARIANT 6: note = N or nothing
  VARIANT 14: note = N, S or G
  VARIANT 9: note = Y, S, A or G
  VARIANT 7: note = V or nothing
  VARIANT 4: note = S, N or Y
  VARIANT 11: note = D, N or T
  VARIANT 10: note = E, G or S
  VARIANT 5: note = S, N or nothing
SGSXXXXXXXXXVX

SEQ ID NO: 106-107 - skipped (2 entries)

SEQ ID NO: 108 - protein, 14 aa, synthetic construct
  REGION 1..14: note = VLCDR1 NS4E3
SGSSSNVGYEDYVN

SEQ ID NO: 109 - protein, 11 aa, synthetic construct
  REGION 1..11: note = VLCDR1 NS3H4
SGSNIAGNGVG

SEQ ID NO: 110 - protein, 14 aa, synthetic construct
  REGION 1..14: note = VLCDR1 NS4F2
SGSSNNVGSGDYVS

SEQ ID NO: 111 - protein, 11 aa, synthetic construct
  REGION 1..11: note = VLCDR1 NS3E5
SGSYIGSTDVG

SEQ ID NO: 112 - protein, 7 aa, synthetic construct
  REGION 1..7: note = VLCDR2 NS4E3
GTTNRPS

SEQ ID NO: 113 - protein, 7 aa, synthetic construct
  REGION 1..7: note = VLCDR2 NS3E5
GSTRRPS

SEQ ID NO: 114 - protein, 7 aa, synthetic construct
  REGION 1..7: note = VLCDR2 NS3H4
RNSNRPS

SEQ ID NO: 115 - protein, 7 aa, synthetic construct
  REGION 1..7: note = VLCDR2 NS4F2
RTTTRAS

SEQ ID NO: 116 - protein, 10 aa, synthetic construct
  REGION 1..10: note = VLCDR3 NS4E3
LSYDRSGSNF

SEQ ID NO: 117 - protein, 10 aa, synthetic construct
  REGION 1..10: note = VLCDR3 NS4F2
ASYDTSNRGI

SEQ ID NO: 118 - protein, 9 aa, synthetic construct
  REGION 1..9: note = VLCDR3 NS3H4
GSYDGTNSF

SEQ ID NO: 119 - protein, 10 aa, synthetic construct
  REGION 1..10: note = VLCDR3 NS3E5
ASYDSNNSIV

SEQ ID NO: 120 - protein, 30 aa, Homo sapiens
SSTGSIDMVDSPQLATLADEVSASLAKQGL

SEQ ID NO: 121 - skipped

SEQ ID NO: 122 - protein, 16 aa, synthetic construct
  REGION 1..16: note = 412 to 441 VHCDR2 consensus
  VARIANT 1: note = A or N
  VARIANT 5: note = S or G
  VARIANT 2: note = S or I
  VARIANT 3: note = G, Y or W
  VARIANT 4: note = S or R
  VARIANT 9: note = Y or E
  VARIANT 8: note = K, T or I
  VARIANT 7: note = N, S or R
XXXXXGXXXYNPALKS

SEQ ID NO: 123 - protein, 8 aa, synthetic construct
  REGION 1..8: note = 412 to 441 VHCDR3 consensus
  VARIANT 2: note = I or G
  VARIANT 3: note = I or V
  VARIANT 4: note = A or G
  VARIANT 5: note = G or S
GXXXXVDV

SEQ ID NO: 124 - protein, 4 aa, synthetic construct
  REGION 1..4: note = VHDR3 412G11
SGGD

SEQ ID NO: 125 - protein, 13 aa, synthetic construct
  REGION 1..13: note = 412 to 441 VLCDR1 consensus
  VARIANT 10: note = N, D or T
  VARIANT 6: note = V or I
  VARIANT 13: note = G or D
  VARIANT 8: note = Y or R
  VARIANT 11: note = Y or F
  VARIANT 3: note = S or G
  VARIANT 4: note = S or N
SGXXNXGXGXXVX

SEQ ID NO: 126-127 - skipped (2 entries)

SEQ ID NO: 128 - protein, 5 aa, synthetic construct
  REGION 1..5: note = VHCDR1 412E10 and 412B9
SDSVA

SEQ ID NO: 129 - protein, 5 aa, synthetic construct
  REGION 1..5: note = VHCDR1 412E6
NYGVG

SEQ ID NO: 130 - protein, 5 aa, synthetic construct
  REGION 1..5: note = VHCDR1 412G11
SYALG

SEQ ID NO: 131 - protein, 16 aa, synthetic construct
  REGION 1..16: note = VHCDR2 412E10 and 412B9
ASGSSGNKYYNPALKS

SEQ ID NO: 132 - protein, 16 aa, synthetic construct
  REGION 1..16: note = VHCDR2 412G11
NIWRGGRIEYNPALKS

SEQ ID NO: 133 - protein, 16 aa, synthetic construct
  REGION 1..16: note = VHCDR2 412E6
NIYSGGSTYYNPALKS

SEQ ID NO: 134 - protein, 8 aa, synthetic construct
  REGION 1..8: note = VHCDR3 412E10 and 412B9
GIIAGVDV

SEQ ID NO: 135 - protein, 8 aa, synthetic construct
  REGION 1..8: note = VHCDR3 412E6
GGVGSVDV

SEQ ID NO: 136 - skipped

SEQ ID NO: 137 - protein, 14 aa, synthetic construct
  REGION 1..14: note = VLCDR1 412E6
SGGRNNIGRGTFVD

SEQ ID NO: 138 - protein, 14 aa, synthetic construct
  REGION 1..14: note = VLCDR1 412B9, 3aD3, 3aA6
SGSSSNVGYGDYVG

SEQ ID NO: 139 - protein, 7 aa, synthetic construct
  REGION 1..7: note = VLCDR2 412E10
GTAIRAS

SEQ ID NO: 140 - protein, 6 aa, synthetic construct
  REGION 1..6: note = VLCDR2 412B9
AASRAS

SEQ ID NO: 141 - protein, 7 aa, synthetic construct
  REGION 1..7: note = VLCDR2 412G11, VLCDR2 3aG3, VLCDR2 3bF4, VLCDR2 CB7, CG11, CE2/E1B8, CC5
GATSRAS

SEQ ID NO: 142 - protein, 7 aa, synthetic construct
  REGION 1..7: note = VLCDR2 412E6
GTDRRPP

SEQ ID NO: 143 - protein, 9 aa, synthetic construct
  REGION 1..9: note = VLCDR3 412E10
ASYQSNYAF

SEQ ID NO: 144 - protein, 10 aa, synthetic construct
  REGION 1..10: note = VLCDR3 412G11
ASYDRSESVV

SEQ ID NO: 145 - protein, 10 aa, synthetic construct
  REGION 1..10: note = VLCDR3 412B9
ASYDSSDGGV

SEQ ID NO: 146 - protein, 11 aa, synthetic construct
  REGION 1..11: note = VLCDR3 412E6
ATYDYSNDMII

SEQ ID NO: 147 - protein, 241 aa, synthetic construct
  REGION 1..241: note = 412E10 amino acid sequence
QVQLQESGPSLVKPSQTLSLTCTVSGFSVISDSVAWVRQAPGKVPEWLGASGSSGNKYYNPALKSRLSITRDTSKSQVSLSLSSVTTEDTAVYYCARGIIAGVDVWGRGLLVTVSSEGKSSGASGESKVDDQAVLTQPSSVSGSLGQRVSITCSGSSSNVGYGNYVGWYQQVPGSAPKLLIYGTAIRASGVPDRFSGSRSGDTATLTITSLQAEDEADYYCASYQSNYAFFGSGTRLTVLG

SEQ ID NO: 148 - protein, 49 aa, Homo sapiens
MAEPRQEFEVMEDHAGTYGLGDRKDQGGYTMHQDQEGDTDAGLKESPLQ

SEQ ID NO: 149 - protein, 5 aa, synthetic construct
  REGION 1..5: note = 1 to 49 VHCDR1 consensus, VHCDR1 3aG3, VHCDR1 3aD3, VHCDR1 3aH6, VHCDR1 3bG4; 49 to 111 VHCDR1 consensus, VHCDR1 3aB7, VHCDR1 3bF4
SNGVG

SEQ ID NO: 150 - protein, 16 aa, synthetic construct
  REGION 1..16: note = 1 to 49 VHDR2 consensus
  VARIANT 10: note = A, S or G
  VARIANT 3: note = S or A
  VARIANT 11: note = N or H
  VARIANT 5: note = S, V or G
  VARIANT 8: note = A, K or V
DIXSXGKXYXXPALKS

SEQ ID NO: 151 - protein, 14 aa, synthetic construct
  REGION 1..14: note = 1 to 49 VHDR3 consensus
  VARIANT 7: note = S or T
  VARIANT 12: note = I or S
CRDGGVXYGYDXDY

SEQ ID NO: 152 - protein, 14 aa, synthetic construct
  REGION 1..14: note = 1 to 49 VLDR1 consensus
  VARIANT 4: note = S or T
  VARIANT 12: note = Y or D
  VARIANT 9: note = G, S or Y
  VARIANT 11: note = N or D
  VARIANT 7: note = I or V
  VARIANT 5: note = S or G
  VARIANT 13: note = L or V
  VARIANT 14: note = S or G
SGSXXNXGXGXXXX

SEQ ID NO: 153 - protein, 7 aa, synthetic construct
  REGION 1..7: note = 1 to 49 VLDR2 consensus
  VARIANT 4: note = S, N or E
  VARIANT 5: note = R or L
  VARIANT 2: note = A or V
GXTXXAS

SEQ ID NO: 154 - protein, 10 aa, synthetic construct
  REGION 1..10: note = 1 to 49 VLDR3 consensus
  VARIANT 1: note = A or G
  VARIANT 10: note = I or V
  VARIANT 6: note = S or N
  VARIANT 3: note = F or Y
  VARIANT 5: note = T, S or D
XSXDXXSGGX

SEQ ID NO: 155 - protein, 16 aa, synthetic construct
  REGION 1..16: note = VHCDR2 3aG3
DISSSGKAYANPALKS

SEQ ID NO: 156 - protein, 16 aa, synthetic construct
  REGION 1..16: note = VHCDR2 3aD3
DISSGGKVYGHPALKS

SEQ ID NO: 157 - protein, 16 aa, synthetic construct
  REGION 1..16: note = VHCDR2 3aH6, VHCDR2 3bF4, VHCDR2 CA2, VHCDR2 CB6, VHCDR2 CA10
DISSVGKKYANPALKS

SEQ ID NO: 158 - protein, 16 aa, synthetic construct
  REGION 1..16: note = VHCDR2 3bG4, CC4
DIASSGKAYSNPALKS

SEQ ID NO: 159 - protein, 14 aa, synthetic construct
  REGION 1..14: note = VHCDR3 3aG3
CRDGGVSYGYDIDY

SEQ ID NO: 160 - protein, 14 aa, synthetic construct
  REGION 1..14: note = VHCDR3 3aD3, CB5
CRDGGVSYGYDSDY

SEQ ID NO: 161 - protein, 14 aa, synthetic construct
  REGION 1..14: note = VHCDR3 3aH6, VHCDR3 3bG4 VHCDR3 3bF4, VHCDR3 CA2, VHCDR3 CB6, VHCDR3 CA10, VHCDR3 CA6, VHCDR3 CC4, VHCDR3 CC5, VHCDR3 CA2
CRDGGVTYGYDIDY

SEQ ID NO: 162 - protein, 16 aa, synthetic construct
  REGION 1..16: note = 297 to 390 VHCDR2 consensus
  VARIANT 7: note = E, T or R
  VARIANT 8: note = E, S or T
  VARIANT 11: note = N or S
  VARIANT 2: note = I or V
  VARIANT 10: note = Y or F
  VARIANT 9: note = G, Y or E
  VARIANT 3: note = D, G or N
  VARIANT 15: note = N or K
  VARIANT 13: note = A or V
  VARIANT 4: note = T, I, Y or S
  VARIANT 12: note = P or S
GXXXDGXXXXXXXLXS

SEQ ID NO: 163 - protein, 14 aa, synthetic construct
  REGION 1..14: note = VLCDR1 3aG3
SGSSSNIGGGNYLS

SEQ ID NO: 164 - skipped

SEQ ID NO: 165 - protein, 14 aa, synthetic construct
  REGION 1..14: note = VLCDR1 3aH6, VLCDR1 CA2
SGSSGNVGYGDYVS

SEQ ID NO: 166 - protein, 14 aa, synthetic construct
  REGION 1..14: note = VLCDR1 3bG4
SGSTSNVGSGNDVS

SEQ ID NO: 167 - protein, 14 aa, synthetic construct
  REGION 1..14: note = 297 to 390 VLCDR1 consensus
  VARIANT 6: note = G or N
  VARIANT 4: note = F, N, S or Y
  VARIANT 10: note = nothing or G
  VARIANT 11: note = nothing or D
  VARIANT 8: note = S, A or G
  VARIANT 7: note = I, S or V
  VARIANT 5: note = I or S
  VARIANT 9: note = S, Y or G
  VARIANT 12: note = nothing or Y
  VARIANT 14: note = G, T or S
SGSXXXXXXXXXVX

SEQ ID NO: 168 - protein, 7 aa, synthetic construct
  REGION 1..7: note = VLCDR2 3aD3
GVTERAS

SEQ ID NO: 169 - protein, 7 aa, synthetic construct
  REGION 1..7: note = VLCDR2 3aH6, VLCDR2 CA2
GATNLAS

SEQ ID NO: 170 - protein, 7 aa, synthetic construct
  REGION 1..7: note = VLCDR2 E2A6, 3bG4, CC4
GATNRAS

SEQ ID NO: 171 - protein, 10 aa, synthetic construct
  REGION 1..10: note = VLCDR3 3aG3
ASFDTSSGGI

SEQ ID NO: 172 - protein, 10 aa, synthetic construct
  REGION 1..10: note = VLCDR3 3aD3
ASYDDSSGGI

SEQ ID NO: 173 - protein, 10 aa, synthetic construct
  REGION 1..10: note = VLCDR3 3aH6, VLCDR3 CA2
ASYDSSSGGV

SEQ ID NO: 174 - protein, 10 aa, synthetic construct
  REGION 1..10: note = VLCDR3 3bG4
GSYDSNSGGI

SEQ ID NO: 175 - protein, 63 aa, Homo sapiens
QTPTEDGSEEPGSETSDAKSTPTAEDVTAPLVDEGAPGKQAAAQPHTEIPEGTTAEEAGIGDT

SEQ ID NO: 176 - protein, 16 aa, synthetic construct
  REGION 1..16: note = 49 to 111 VHCDR2 consensus
  VARIANT 5: note = V or A
  VARIANT 2: note = I or K
  VARIANT 10: note = A or G
  VARIANT 8: note = K or T
DXSSXSKXYXNPALKS

SEQ ID NO: 177 - protein, 14 aa, synthetic construct
  REGION 1..14: note = 49 to 111 VHCDR3 consensus
  VARIANT 12: note = I or V
CRDGGVTYGYDXDY

SEQ ID NO: 178 - protein, 14 aa, synthetic construct
  REGION 1..14: note = 49 to 111 VLCDR1 consensus
  VARIANT 9: note = L or Y
  VARIANT 10: note = R or G
  VARIANT 14: note = T or S
  VARIANT 11: note = N or D
  VARIANT 12: note = Y or V
SGSSSNVGXXXXVX

SEQ ID NO: 179 - protein, 7 aa, synthetic construct
  REGION 1..7: note = 49 to 111 VLCDR2 consensus
  VARIANT 2: note = A or T
  VARIANT 4: note = S or T
GXTXRAS

SEQ ID NO: 180 - protein, 10 aa, synthetic construct
  REGION 1..10: note = 49 to 111 VLCDR3 consensus
  VARIANT 6: note = N or D
  VARIANT 10: note = V or I
  VARIANT 3: note = A or F
  VARIANT 5: note = T or S
  VARIANT 7: note = D or S
ASXDXXXGGX

SEQ ID NO: 181 - skipped

SEQ ID NO: 182 - protein, 16 aa, synthetic construct
  REGION 1..16: note = VHCDR2 3aB7, CB12, CC3, CA3, CD1
DKSSAGKTYGNPALKS

SEQ ID NO: 183 - skipped

SEQ ID NO: 184 - protein, 14 aa, synthetic construct
  REGION 1..14: note = VHCDR3 3aB7, CA3
CRDGGVTYGYDVDY

SEQ ID NO: 185 - protein, 14 aa, synthetic construct
  REGION 1..14: note = VLCDR1 3bF4
SGSSSNVGLRNYVT

SEQ ID NO: 186 - protein, 14 aa, synthetic construct
  REGION 1..14: note = VLCDR1 3aB7
SGSSSNVGYGDVVS

SEQ ID NO: 187 - protein, 5 aa, synthetic construct
  REGION 1..5: note = VHCDR1 S1D5
SHSVG

SEQ ID NO: 188 - protein, 7 aa, synthetic construct
  REGION 1..7: note = VLCDR2 3aB7, CC3, CD1
GTTTRAS

SEQ ID NO: 189 - protein, 10 aa, synthetic construct
  REGION 1..10: note = VLCDR3 3bF4
ASADTNDGGV

SEQ ID NO: 190 - protein, 10 aa, synthetic construct
  REGION 1..10: note = VLCDR3 3aB7
ASFDSDSGGI

SEQ ID NO: 191 - protein, 11 aa, Homo sapiens
GKTKIATPRGA

SEQ ID NO: 192 - protein, 5 aa, synthetic construct
  REGION 1..5: note = 147 to 157 VHCDR1 consensus
  VARIANT 5: note = I or G
SNAVX

SEQ ID NO: 193 - protein, 16 aa, synthetic construct
  REGION 1..16: note = 147 to 157 VHCDR2 consensus
  VARIANT 4: note = V or I
  VARIANT 11: note = D or N
  VARIANT 15: note = K or E
  VARIANT 8: note = A or T
LIDXDGDXAYXPALXS

SEQ ID NO: 194 - protein, 13 aa, synthetic construct
  REGION 1..13: note = 147 to 157 VHCDR3 consensus
  VARIANT 10: note = D or S
  VARIANT 9: note = S or D
  VARIANT 1: note = D or H
  VARIANT 3: note = G or D
  VARIANT 4: note = S or K
  VARIANT 8: note = V or A
XYXXWGYXXXIDY

SEQ ID NO: 195 - protein, 14 aa, synthetic construct
  REGION 1..14: note = 147 to 157 VLCDR1 consensus
  VARIANT 12: note = D or Y
  VARIANT 11: note = A or D
  VARIANT 9: note = G or Y
  VARIANT 4: note = D or S
  VARIANT 8: note = I or G
  VARIANT 5: note = nothing or S
  VARIANT 6: note = nothing or N
  VARIANT 7: note = nothing or V
SGSXXXXXXGXXVG

SEQ ID NO: 196-197 - skipped (2 entries)

SEQ ID NO: 198 - protein, 5 aa, synthetic construct
  REGION 1..5: note = VHCDR1 3aD6, CB11, CC7, CA1, CD2
SNAVI

SEQ ID NO: 199 - protein, 5 aa, synthetic construct
  REGION 1..5: note = VHCDR1 NS1B2
DYGIG

SEQ ID NO: 200 - protein, 16 aa, synthetic construct
  REGION 1..16: note = VHCDR2 3aD6, CB11, CC7, CD2
LIDVDGDAAYDPALKS

SEQ ID NO: 201 - protein, 16 aa, synthetic construct
  REGION 1..16: note = VHCDR2 3aA6
LIDIDGDTAYNPALES

SEQ ID NO: 202 - protein, 13 aa, synthetic construct
  REGION 1..13: note = VHCDR3 3aD6, CB11, CC7, CD2
DYGSWGYVSDIDY

SEQ ID NO: 203 - protein, 13 aa, synthetic construct
  REGION 1..13: note = VHCDR3 3aA6
HYDKWGYADSIDY

SEQ ID NO: 204 - protein, 11 aa, synthetic construct
  REGION 1..11: note = VLCDR1 3aD6
SGSDIGGADVG

SEQ ID NO: 205 - protein, 16 aa, synthetic construct
  REGION 1..16: note = VHDR2 NS1B2
GINYDGRTEYNSALKS

SEQ ID NO: 206 - protein, 7 aa, synthetic construct
  REGION 1..7: note = VLCDR2 3aD6
DNDNRPS

SEQ ID NO: 207 - protein, 7 aa, synthetic construct
  REGION 1..7: note = VLCDR2 3aA6, CA6
DATTRAS

SEQ ID NO: 208 - protein, 10 aa, synthetic construct
  REGION 1..10: note = VLCDR3 3aD6
GTYSGANYGI

SEQ ID NO: 209 - protein, 10 aa, synthetic construct
  REGION 1..10: note = VLCDR3 3aA6
ASYQNERSGV

SEQ ID NO: 210 - protein, 17 aa, synthetic construct
  REGION 1..17: note = VHCDR3 S1A12, S1A5
TYRSDGYAYGYVQAIDY

SEQ ID NO: 211 - protein, 14 aa, synthetic construct
  REGION 1..14: note = VHCDR3 NS1B2
DSKGGWGHVYQFDY

SEQ ID NO: 212 - protein, 11 aa, synthetic construct
  REGION 1..11: note = VLCDR1 NS2B6
SGSNIGSASVT

SEQ ID NO: 213 - protein, 14 aa, synthetic construct
  REGION 1..14: note = VLCDR1 NS1B2
SGSSSNVGYGDYVS

SEQ ID NO: 214 - protein, 7 aa, synthetic construct
  REGION 1..7: note = VLCDR2 NS2B6
RNRNRPS

SEQ ID NO: 215 - protein, 7 aa, synthetic construct
  REGION 1..7: note = VLCDR2 NS1B2
DATNRAS

SEQ ID NO: 216 - protein, 10 aa, synthetic construct
  REGION 1..10: note = VLCDR3 NS1B2
ASHDNRISAV

SEQ ID NO: 217 - protein, 10 aa, synthetic construct
  REGION 1..10: note = VLCDR3 NS2B6
GSYQSWGSGV

SEQ ID NO: 218 - protein, 249 aa, synthetic construct
  REGION 1..249: note = S1E12 amino acid sequence
QVQLQESGPSLVKPSQTLSLTCTVSGFSLGSNSVGWVRQAPGKAPEWVAGIDTDGEEGYNPALNSRLSITRDTSKSQVSLSLSSVTSEDTAVYYCGRSYRADGLAYGYVQAIDYWGPGLLVTVSSEGKSSGASGESKVDDRVVRTQPSSVSGSLGQRVSITCSGSFIGISSVGWFQQLPGSGLRTIIVASDGRPSGVPDRFSMSKSGNTATLTISSLQAEDEADYFCGSSDRTQYTGVFGSGTRLTVLG

SEQ ID NO: 219 - DNA, 747 bp, synthetic construct
  misc_feature 1..747: note = S1E12 nucleotide sequence
  source 1..747: mol_type = other DNA
caggtgcagttgcaggagtcgggacccagcctggtgaagccctcacagaccctctccctcacctgcacggtctccggattctcattaggcagcaattctgtaggctgggtccgccaggctccaggaaaggcgccggagtgggttgctggtatagatactgatggagaagaaggctataatccagcccttaactcccggctcagcatcaccagggacacctccaagagtcaggtctctttgtcattgagcagcgtgacaagtgaggacacggccgtgtactactgtggaagaagttatagggctgatggtcttgcttacggttatgtccaagccatcgactactggggcccaggactcctggtcaccgtctcctcagaaggtaaatcttctggcgcgtctggcgagtctaaagtggatgacagggtcgtgcggactcaaccgtcctccgtgtctgggtccctgggccagagggtctccatcacctgctctggaagcttcatcggtattagtagtgtaggctggttccaacagctcccaggatcgggcctcagaaccatcatcgtggcgagtgacggtcgaccctcaggggtccccgaccgattctctatgtccaaatcgggcaacacagccaccctgaccatcagctcgctccaggctgaggacgaggccgattatttctgtggaagtagtgataggactcaatatactggagttttcggcagcgggaccaggctgaccgtcctgggt

SEQ ID NO: 220 - protein, 249 aa, synthetic construct
  REGION 1..249: note = NS2A1 amino acid sequence
QVQLQESGPSLVKPSQTLSLTCTVSGFSLSTNSVGWVRQAPGKAPEWVAGIDTDGEEGFNPVLKSRLSITRDTSKSQVSLSLSNVTSEDTAVYYCGRSYRTDGLAYGYVQAIDYWGPGLLVTISSEGKSSGASGESKVDDQSVLTQPSSVSGSPGQTVSITCSGSYIGSSGVGWFQQLPGSGLRTIIVASDGRPSGVPDGFSMSKSGNTATLTISSLQAEDEADYFCGSSDRTQYTGLFGSGTRLTVLG

SEQ ID NO: 221 - protein, 13 aa, synthetic construct
  REGION 1..13: note = E2E8, E1E8 epitope, residues 379-391
RENAKAKTDHGAE

SEQ ID NO: 222-223 - skipped (2 entries)

SEQ ID NO: 224 - protein, 16 aa, synthetic construct
  REGION 1..16: note = 379-391 VHDR2 consensus
  VARIANT 7: note = T or G
  VARIANT 10: note = Y or D
  VARIANT 9: note = D or E
  VARIANT 8: note = I or T
TMRSGGXXXXNPALKS

SEQ ID NO: 225 - protein, 9 aa, synthetic construct
  REGION 1..9: note = 379-391 VHDR3 consensus
  VARIANT 6: note = D, I or V
  VARIANT 7: note = R or H
GYLSGXXYA

SEQ ID NO: 226 - protein, 14 aa, synthetic construct
  REGION 1..14: note = 379-391 VLDR1 consensus
  VARIANT 7: note = I or V
  VARIANT 4: note = R or S
  VARIANT 9: note = Y, D or A
  VARIANT 11: note = N, D or R
  VARIANT 6: note = D or N
  VARIANT 14: note = S or G
SGSXSXXGXGXYVX

SEQ ID NO: 227-228 - skipped (2 entries)

SEQ ID NO: 229 - protein, 5 aa, synthetic construct
  REGION 1..5: note = VHCDR1 E2E8
DRGVA

SEQ ID NO: 230 - protein, 5 aa, synthetic construct
  REGION 1..5: note = VHCDR1 E1E8, E2A6
DWGVA

SEQ ID NO: 231 - protein, 5 aa, synthetic construct
  REGION 1..5: note = VHCDR1 E2B7
SWGVA

SEQ ID NO: 232 - protein, 16 aa, synthetic construct
  REGION 1..16: note = VHCDR2 E2E8
TMRSGGTIDYNPALKS

SEQ ID NO: 233 - protein, 16 aa, synthetic construct
  REGION 1..16: note = VHCDR2 E2B7
TMRSGGGTEYNPALKS

SEQ ID NO: 234 - protein, 16 aa, synthetic construct
  REGION 1..16: note = VHCDR2 E1E8, E2A6
TMRSGGTTDDNPALKS

SEQ ID NO: 235 - protein, 9 aa, synthetic construct
  REGION 1..9: note = VHCDR3 E2E8
GYLSGDRYA

SEQ ID NO: 236 - protein, 9 aa, synthetic construct
  REGION 1..9: note = VHCDR3 E2B7
GYLSGIHYA

SEQ ID NO: 237 - protein, 9 aa, synthetic construct
  REGION 1..9: note = VHCDR3 E1E8, E2A6
GYLSGVHYA

SEQ ID NO: 238 - protein, 14 aa, synthetic construct
  REGION 1..14: note = VLCDR1 E2E8
SGSRSDIGYGNYVS

SEQ ID NO: 239 - protein, 14 aa, synthetic construct
  REGION 1..14: note = VLCDR1 E2A6
SGSSSNVGAGNYVG

SEQ ID NO: 240 - protein, 14 aa, synthetic construct
  REGION 1..14: note = VLCDR1 E2B7
SGSSSNVGDGDYVG

SEQ ID NO: 241 - protein, 14 aa, synthetic construct
  REGION 1..14: note = VLCDR1 E1E8
SGSSSNVGDGRYVS

SEQ ID NO: 242 - protein, 7 aa, synthetic construct
  REGION 1..7: note = VLCDR2 E2E8
DTNTRAS

SEQ ID NO: 243 - protein, 7 aa, synthetic construct
  REGION 1..7: note = VLCDR2 E1E8
DTTSRAS

SEQ ID NO: 244 - protein, 7 aa, synthetic construct
  REGION 1..7: note = VLCDR2 E2B7
SARNRAS

SEQ ID NO: 245 - protein, 10 aa, synthetic construct
  REGION 1..10: note = VLCDR3 E2E8
ANIDSSRSHL

SEQ ID NO: 246 - protein, 10 aa, synthetic construct
  REGION 1..10: note = VLCDR3 E1E8
ASIDSGNNLL

SEQ ID NO: 247 - protein, 10 aa, synthetic construct
  REGION 1..10: note = VLCDR3 E2A6, E2B7
ASIDTSRSHI

SEQ ID NO: 248 - protein, 243 aa, synthetic construct
  REGION 1..243: note = E2E8 amino acid sequence
QVQLQESGPSLVKPSQTLSLTCTVSGFSLTDRGVAWVRQAPGKALEWVGTMRSGGTIDYNPALKSRLSITRDTSKSQVFLSLSSVTTEDMAMYYCARGYLSGDRYAWGRGLLVTVSSEGKSSGASGESKVDDQAVLTQPSSVSKSLGQSVSIACSGSRSDIGYGNYVSWFQQIPGSAPKLLIYDTNTRASGVPDRFSGARSGNTATLTINSLQAEDEADYYCANIDSSRSHLFGSGTRLTVLG

SEQ ID NO: 249 - DNA, 729 bp, synthetic construct
  misc_feature 1..729: note = E2E8 nucleotide sequence
  source 1..729: mol_type = other DNA
caggtgcagctgcaggagtcgggacccagcctggtgaagccctcacagaccctctccctcacctgcacggtctctggattctcattaaccgaccgtggtgtagcctgggtccgccaggctccaggaaaggcactggagtgggttggtactatgcgtagtggtggaacgatagactataacccggccctgaaatcccggctcagcatcaccagggacacctccaagagccaagttttcctgtcactgagcagcgtcacaactgaggacatggccatgtactactgtgccagaggttatttgagcggtgatcgttatgcctggggccgaggactcctggtcaccgtctcctcagaaggtaaatcttctggcgcgtctggcgagtctaaagtggatgaccaggctgtgctgactcagccgtcctccgtgtccaagtccctgggccagagtgtctccatcgcctgctctggaagcaggagcgacattggatatggtaattatgtgagctggttccaacagatcccaggatcagcccccaaactccttatttatgatacaaacactcgggcctcgggggtccccgaccgattctccggcgccaggtctggcaacacagcaacactgaccatcaactcgctccaggctgaggacgaggccgattattactgtgcaaatattgacagtagtcgcagtcatcttttcggcagtggcaccagactgaccgtcctgggt

SEQ ID NO: 250 - protein, 243 aa, synthetic construct
  REGION 1..243: note = E1E8 amino acid sequence
QVQLQESGPSLVKPSQTLSLTCTVSGFSLTDWGVAWVRQAPGKALEWLGTMRSGGTTDDNPALKSRLSITRDTSKSQVSLSLSSVTTEDMAMYYCARGYLSGVHYAWGRGLLVTVSSEGKSSGASGESKVDDQAVLTQPSSVSGSLGQSVSITCSGSSSNVGDGRYVSWFQQVPGSAPKLLIYDTTSRASGVPDRFSGSRSGNTATLIITSLQAEDEADYYCASIDSGNNLLFGSGTRLTVLG

SEQ ID NO: 251 - DNA, 729 bp, synthetic construct
  misc_feature 1..729: note = E1E8 nucleotide sequence
  source 1..729: mol_type = other DNA
caggtgcagctgcaggagtcgggacccagcctggtgaagccctcacagaccctctccctcacctgcacggtctctggattctcattaactgactggggtgtagcttgggtccgccaggctccaggaaaggcactggagtggcttggtacgatgcgtagtggtgggactacagacgataacccggccctgaaatcccgcctcagcatcaccagggacacctctaagagccaagtctccctgtcactgagcagcgtgacaactgaggacatggccatgtactactgtgccagaggttatttgagtggtgtgcattatgcctggggccgaggactcctggtcaccgtctcctcagaaggtaaatcttctggcgcgtctggcgagtctaaagtggatgaccaggctgtgctgactcagccgtcctccgtatctgggtccctgggccagagtgtctccatcacctgctctggaagcagcagcaacgtgggagatggtagatatgtgagctggttccaacaggtcccaggatcagcccccaaactcctcatctatgatacaaccagtcgagcctcgggggttcccgaccgattctccggctccaggtctggcaacacagcgactctcatcatcacctcgctccaggctgaggacgaggccgattattactgtgcatctattgacagcggtaacaatcttcttttcggcagcggcaccaggctgaccgtcctgggt

SEQ ID NO: 252 - protein, 243 aa, synthetic construct
  REGION 1..243: note = E2A6 amino acid sequence
QVQLQESGPSLVKPSQTLSLTCTVSGFSLTDWGVAWVRQAPGKALEWLGTMRSGGTTDDNPALKSRLSITRDTSKSQVSLSLSSVTTEDMAMYYCARGYLSGVHYAWGRGLLVTVSSEGKSSGASGESKVDDRVVRTQPSSVSKSLGQSVSITCSGSSSNVGAGNYVGWFQQVPGSAPKLLIYGATNRASGVPARFSGSKSGVTATLTITSLQAEDEADYYCASIDTSRSHIFGSGTRLTVLG

SEQ ID NO: 253 - DNA, 729 bp, synthetic construct
  misc_feature 1..729: note = E2A6 nucleotide sequence
  source 1..729: mol_type = other DNA
caggtgcagctgcaggagtcgggacccagcctggtgaagccctcacagaccctctccctcacctgcacggtctctggattctcattaactgactggggtgtagcttgggtccgccaggctccaggaaaggcactggagtggcttggtacgatgcgtagtggtgggactacagacgataacccggccctgaaatcccgcctcagcatcaccagggacacctctaagagccaagtctccctgtcactgagcagcgtgacaactgaggacatggccatgtactactgtgccagaggttatttgagtggtgtgcattatgcctggggccgaggactcctggtcaccgtctcctcagaaggtaaatcttctggcgcgtctggcgagtctaaagtggatgaccgggtcgtgcggactcagccgtcctccgtgtccaagtccctgggccagagtgtctccatcacctgctctggaagcagcagcaacgttggagctggtaattatgtgggctggttccaacaggtcccaggatcagcccccaaactcctcatctatggtgcaaccaatcgagcctcgggggtccccgcccgattctcaggctccaagtctggcgtcacagcgactctaaccatcacctcgctccaggctgaggacgaggccgattattactgtgcatctattgacaccagtcgctctcacattttcggcagcgggaccaggctgaccgtcctgggt

SEQ ID NO: 254 - protein, 243 aa, synthetic construct
  REGION 1..243: note = E2B7 amino acid sequence
QVQLQESGPSLVKPSQTLSLTCTVSGFSLTSWGVAWVRQAPGKALEWLGTMRSGGGTEYNPALKSRLSITRDTSKSQVSLSLSSVTTEDMAMYYCARGYLSGIHYAWGRGLLVSVSSEGKSSGASGESKVDDQAVLTQLSSVSGSLGQRVSITCSGSSSNVGDGDYVGWFQQLPGSAPKLLIYSARNRASGVPDRFSGSRSGNTATLTITSLQAEDEADYYCASIDTSRSHIFGSGTRLTVLG

SEQ ID NO: 255 - protein, 126 aa, Homo sapiens
SLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSS

SEQ ID NO: 256 - protein, 11 aa, synthetic construct
  REGION 1..11: note = VLCDR1 CB11
SGSNIGSNDVG

SEQ ID NO: 257 - protein, 7 aa, synthetic construct
  REGION 1..7: note = VLCDR2 CB11
DNNNRPS

SEQ ID NO: 258 - protein, 11 aa, synthetic construct
  REGION 1..11: note = VLCDR3 CB11
GGYAGSSSNFL

SEQ ID NO: 259 - protein, 5 aa, synthetic construct
  REGION 1..5: note = VHCDR1 CA2, CB6, CB12, CC3, CA3, CA10, CA6, CB5, CC4, CD1, CC5
SNGVG

SEQ ID NO: 260 - protein, 238 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSS

SEQ ID NO: 261 - protein, 14 aa, synthetic construct
  REGION 1..14: note = VLCDR1 CB6
SGSSSNIGTGNYVG

SEQ ID NO: 262 - protein, 7 aa, synthetic construct
  REGION 1..7: note = VLCDR2 CB6
GAVTRAS

SEQ ID NO: 263 - protein, 10 aa, synthetic construct
  REGION 1..10: note = VLCDR3 CB6, CB7
ASYDSTSGGV

SEQ ID NO: 264 - protein, 319 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVT

SEQ ID NO: 265 - protein, 17 aa, synthetic construct
  REGION 1..17: note = VHCDR3 CA7, CA12
GSYYHGGGNGMVDFFDY

SEQ ID NO: 266 - protein, 5 aa, synthetic construct
  REGION 1..5: note = VHCDR1 CA8
SNAVV

SEQ ID NO: 267 - protein, 16 aa, synthetic construct
  REGION 1..16: note = VHCDR2 CA8
AIDKDGDTIYNPALKS

SEQ ID NO: 268 - protein, 12 aa, synthetic construct
  REGION 1..12: note = VHCDR3 CA8
DPSGWGYPDVDY

SEQ ID NO: 269 - protein, 11 aa, synthetic construct
  REGION 1..11: note = VLCDR1 CA8
SGTYIGSSDVG

SEQ ID NO: 270 - protein, 7 aa, synthetic construct
  REGION 1..7: note = VLCDR2 CA8
GTSSRPS

SEQ ID NO: 271 - protein, 11 aa, synthetic construct
  REGION 1..11: note = VLCDR3 CA8
ATYESSYHNSV

SEQ ID NO: 272 - protein, 5 aa, synthetic construct
  REGION 1..5: note = VHCDR1 CB10, CA11
SNTVA

SEQ ID NO: 273 - protein, 16 aa, synthetic construct
  REGION 1..16: note = VHCDR2 CB10, CA11
EINSGGSTYYNPALKS

SEQ ID NO: 274 - protein, 9 aa, synthetic construct
  REGION 1..9: note = VHCDR3 CB10, CA11
GARSTYAAY

SEQ ID NO: 275 - protein, 14 aa, synthetic construct
  REGION 1..14: note = VLCDR1 CB10
SGSSSDVGYSTWVY

SEQ ID NO: 276 - protein, 7 aa, synthetic construct
  REGION 1..7: note = VLCDR2 CB10
HISNRAS

SEQ ID NO: 277 - protein, 11 aa, synthetic construct
  REGION 1..11: note = VLCDR3 CB10
AAYDSSNNVWI

SEQ ID NO: 278 - protein, 13 aa, Homo sapiens
DHAGTYGLGDRKD

SEQ ID NO: 279 - protein, 5 aa, synthetic construct
  REGION 1..5: note = VHCDR1 CB7
NYRVG

SEQ ID NO: 280 - protein, 16 aa, synthetic construct
  REGION 1..16: note = VHCDR2 CB7
NIRSGGTTWYNPALKS

SEQ ID NO: 281 - protein, 11 aa, synthetic construct
  REGION 1..11: note = VHCDR3 CB7
DSSGDLYAYDY

SEQ ID NO: 282 - protein, 14 aa, synthetic construct
  REGION 1..14: note = VLCDR1 CB7
SGSSSNVGYGNYMA

SEQ ID NO: 283 - protein, 13 aa, Homo sapiens
ADGKTKIATPRGA

SEQ ID NO: 284 - protein, 7 aa, synthetic construct
  REGION 1..7: note = VLCDR2 CC7
DNNDRPS

SEQ ID NO: 285 - protein, 10 aa, synthetic construct
  REGION 1..10: note = VLCDR3 CC7
ASYDTSNIGL

SEQ ID NO: 286 - protein, 14 aa, synthetic construct
  REGION 1..14: note = VHCDR3 CB12, CC3, CD1
CRDGGVSYGYDVDY

SEQ ID NO: 287 - protein, 13 aa, synthetic construct
  REGION 1..13: note = VLCDR1 CB12
SGSSSNVGGDYVG

SEQ ID NO: 288 - protein, 7 aa, synthetic construct
  REGION 1..7: note = VLCDR2 CB12, CA1
DTTSRPS

SEQ ID NO: 289 - protein, 10 aa, synthetic construct
  REGION 1..10: note = VLCDR3 CB12
ASVDKTTGGV

SEQ ID NO: 290 - protein, 14 aa, synthetic construct
  REGION 1..14: note = VLCDR1 CC3, CD1
SGSSSNVGYGTYVS

SEQ ID NO: 291 - protein, 10 aa, synthetic construct
  REGION 1..10: note = VLCDR3 CC3, CD1
ASYDTGSGGV

SEQ ID NO: 292 - protein, 11 aa, synthetic construct
  REGION 1..11: note = VLCDR1 CC7
SGSYITGSSVG

SEQ ID NO: 293 - protein, 155 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPR

SEQ ID NO: 294 - protein, 73 aa, Homo sapiens
RGAAPPGQKGQANATRIPAKTPPAPKTPPSSGEPPKSGDRSGYSSPGSPGTPGSRSRTPSLPTPPTREPKKVA

SEQ ID NO: 295 - protein, 84 aa, Homo sapiens
RGAAPPGQKGQANATRIPAKTPPAPKTPPSSGEPPKSGDRSGYSSPGSPGTPGSRSRTPSLPTPPTREPKKVAVVRTPPKSPSS

SEQ ID NO: 296 - protein, 16 aa, synthetic construct
  REGION 1..16: note = VHCDR2 CA1
DIRADGATNYNAALKS

SEQ ID NO: 297 - protein, 17 aa, synthetic construct
  REGION 1..17: note = VHCDR3 CA1
PGNYYYGAGRDVARLAD

SEQ ID NO: 298 - protein, 14 aa, synthetic construct
  REGION 1..14: note = VLCDR1 CA1
SGSSSNIGGGNAVG

SEQ ID NO: 299 - protein, 11 aa, synthetic construct
  REGION 1..11: note = VLCDR3 CA1
AAMDSSSLIGV

SEQ ID NO: 300 - protein, 78 aa, Homo sapiens
GEPPKSGDRSGYSSPGSPGTPGSRSRTPSLPTPPTREPKKVAVVRTPPKSPSSAKSRLQTAPVPMPDLKNVKSKIGST

SEQ ID NO: 301 - protein, 14 aa, synthetic construct
  REGION 1..14: note = VLCDR1 CA3
SGSSGNIGYDDYVS

SEQ ID NO: 302 - protein, 7 aa, synthetic construct
  REGION 1..7: note = VLCDR2 CA3
GATRRSS

SEQ ID NO: 303 - protein, 10 aa, synthetic construct
  REGION 1..10: note = VLCDR3 CA3
ASYDSSGGGV

SEQ ID NO: 304 - protein, 165 aa, Homo sapiens
GEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRV

SEQ ID NO: 305 - protein, 11 aa, synthetic construct
  REGION 1..11: note = VLCDR1 CD2
SGSNIGDADVG

SEQ ID NO: 306 - protein, 7 aa, synthetic construct
  REGION 1..7: note = VLCDR2 CD2
YNENRPS

SEQ ID NO: 307 - protein, 12 aa, synthetic construct
  REGION 1..12: note = VLCDR3 CD2
GSYAGDTYNHGV

SEQ ID NO: 308 - protein, 110 aa, Homo sapiens
AKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKD

SEQ ID NO: 309 - protein, 94 aa, Homo sapiens
LKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDN

SEQ ID NO: 310 - protein, 5 aa, synthetic construct
  REGION 1..5: note = VHCDR1 CG11, CE2/E1B8
NYPVG

SEQ ID NO: 311 - protein, 16 aa, synthetic construct
  REGION 1..16: note = VHCDR2 CG11, CE2/E1B8
NIENDGSANYASALKS

SEQ ID NO: 312 - protein, 16 aa, synthetic construct
  REGION 1..16: note = VHCDR3 CG11, CE2/E1B8
EFGGSDGYTYFVDIDY

SEQ ID NO: 313 - protein, 14 aa, synthetic construct
  REGION 1..14: note = VLCDR1 CG11, CE2/E1B8
SGSSSNVGYGNYVS

SEQ ID NO: 314 - protein, 10 aa, synthetic construct
  REGION 1..10: note = VLCDR3 CG11, CE2/E1B8
ASYDGSSSGV

SEQ ID NO: 315 - protein, 43 aa, Homo sapiens
IINKKLDLSNVQSKCGSKDNIKHVPGGGSVQIVYKPVDLSKVT

SEQ ID NO: 316 - protein, 14 aa, synthetic construct
  REGION 1..14: note = VLCDR1 CA10
SGSSSNVGYGNYVT

SEQ ID NO: 317 - protein, 7 aa, synthetic construct
  REGION 1..7: note = VLCDR2 CA10
DATTRVS

SEQ ID NO: 318 - protein, 10 aa, synthetic construct
  REGION 1..10: note = VLCDR3 CA10
AAHDSSSGGV

SEQ ID NO: 319 - protein, 13 aa, Homo sapiens
TSKCGSLGNIHHK

SEQ ID NO: 320 - protein, 13 aa, synthetic construct
  REGION 1..13: note = CE2/E1B8 general epitope
  VARIANT 1..4
  VARIANT 11..13
XXXXGSLGNIXXX

SEQ ID NO: 321 - protein, 30 aa, Homo sapiens
KPGGGQVEVKSEKLDFKDRVQSKIGSLDNI

SEQ ID NO: 322 - protein, 43 aa, Homo sapiens
DRVQSKIGSLDNITHVPGGGNKKIETHKLTFRENAKAKTDHGA

SEQ ID NO: 323 - protein, 16 aa, synthetic construct
  REGION 1..16: note = VHCDR2 CA6
DKSSGGKTYGNPALKS

SEQ ID NO: 324 - protein, 14 aa, synthetic construct
  REGION 1..14: note = VLCDR1 CA6
SGSRNNIGYGNHVG

SEQ ID NO: 325 - protein, 10 aa, synthetic construct
  REGION 1..10: note = VLCDR3 CA6
ASFDRGSGGI

SEQ ID NO: 326 - protein, 94 aa, Homo sapiens
DRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 327 - protein, 14 aa, synthetic construct
  REGION 1..14: note = VLCDR1 CA11
SGSGSNIGAGNWVS

SEQ ID NO: 328 - protein, 7 aa, synthetic construct
  REGION 1..7: note = VLCDR2 CA11
GATSRPS

SEQ ID NO: 329 - protein, 10 aa, synthetic construct
  REGION 1..10: note = VLCDR3 CA11
AAYDSGSSIV

SEQ ID NO: 330 - protein, 13 aa, Homo sapiens
GSLDNITHVPGGG

SEQ ID NO: 331 - protein, 13 aa, synthetic construct
  REGION 1..13: note = CA4 general epitope
  VARIANT 1..3
  VARIANT 5
  VARIANT 9
  VARIANT 11..13
XXXDXITHXPXXX

SEQ ID NO: 332 - protein, 33 aa, Homo sapiens
NITHVPGGGNKKIETHKLTFRENAKAKTDHGAE

SEQ ID NO: 333 - protein, 31 aa, Homo sapiens
ITHVPGGGNKKIETHKLTFRENAKAKTDHGA

SEQ ID NO: 334 - protein, 5 aa, synthetic construct
  REGION 1..5: note = VHCDR1 CB3
SVAVN

SEQ ID NO: 335 - protein, 16 aa, synthetic construct
  REGION 1..16: note = VHCDR2 CB3
GIISNGGTGYNPALKS

SEQ ID NO: 336 - protein, 10 aa, synthetic construct
  REGION 1..10: note = VHCDR3 CB3
GVEWEGSMDY

SEQ ID NO: 337 - protein, 14 aa, synthetic construct
  REGION 1..14: note = VLCDR1 CB3
SGSSSNVGAGSYVG

SEQ ID NO: 338 - protein, 7 aa, synthetic construct
  REGION 1..7: note = VLCDR2 CB3
GATKRAS

SEQ ID NO: 339 - protein, 10 aa, synthetic construct
  REGION 1..10: note = VLCDR3 CB3
VSYQTDFTLV

SEQ ID NO: 340 - protein, 65 aa, Homo sapiens
QTPTEDGSEEPGSETSDAKSTPTAEDVTAPLVDEGAPGKQAAAQPHTEIPEGTTAEEAGIGDTPS

SEQ ID NO: 341 - protein, 16 aa, synthetic construct
  REGION 1..16: note = VHCDR2 CB5
DITSGGRTYGNLALKS

SEQ ID NO: 342 - protein, 14 aa, synthetic construct
  REGION 1..14: note = VLCDR1 CB5
SGSSSNVGSGDHVN

SEQ ID NO: 343 - protein, 7 aa, synthetic construct
  REGION 1..7: note = VLCDR2 CB5
RTTNRAS

SEQ ID NO: 344 - protein, 10 aa, synthetic construct
  REGION 1..10: note = VLCDR3 CB5
ASHDNNSGGV

SEQ ID NO: 345 - protein, 107 aa, Homo sapiens
QTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPR

SEQ ID NO: 346 - protein, 19 aa, synthetic construct
  REGION 1..19: note = VLCDR1 CC4
SGSSSNVGYTNLGYSNLVT

SEQ ID NO: 347 - protein, 10 aa, synthetic construct
  REGION 1..10: note = VLCDR3 CC4
ASYDSSNGGI

SEQ ID NO: 348 - protein, 190 aa, Homo sapiens
QTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSS

SEQ ID NO: 349 - protein, 16 aa, synthetic construct
  REGION 1..16: note = VHCDR2 CC5
DISSVGKKYASPALKS

SEQ ID NO: 350 - protein, 10 aa, synthetic construct
  REGION 1..10: note = VLCDR3 CC5
ASYDSSNGGV

SEQ ID NO: 351 - protein, 13 aa, Homo sapiens
THKLTFRENAKAK

SEQ ID NO: 352 - protein, 13 aa, synthetic construct
  REGION 1..13: note = VLCDR1 MD9
SGSSSNVGIYDVS

SEQ ID NO: 353 - protein, 7 aa, synthetic construct
  REGION 1..7: note = VLCDR2 MD9
GTNNRPS

SEQ ID NO: 354 - protein, 10 aa, synthetic construct
  REGION 1..10: note = VLCDR3 MD9
AAGDSSTIAV

SEQ ID NO: 355 - protein, 31 aa, Homo sapiens
VQIINKKLDLSNVQSKCGSKDNIKHVPGGGS

SEQ ID NO: 356 - protein, 32 aa, Homo sapiens; identical to SEQ ID NO: 82

SEQ ID NO: 357 - protein, 5 aa, synthetic construct
  REGION 1..5: note = VHCDR1 NS1G7
SYGVG

SEQ ID NO: 358 - protein, 16 aa, synthetic construct
  REGION 1..16: note = VHCDR2 NS1G7
SISSGGTTFYNPALKS

SEQ ID NO: 359 - protein, 18 aa, synthetic construct
  REGION 1..18: note = VHCDR3 NS1G7
DVHIYYNDYGAAYGDRDY

SEQ ID NO: 360 - protein, 14 aa, synthetic construct
  REGION 1..14: note = VLCDR1 NS1G7
SGSSSNIGGGNYVS

SEQ ID NO: 361 - protein, 7 aa, synthetic construct
  REGION 1..7: note = VLCDR2 NS1G7
GTTSRAS

SEQ ID NO: 362 - protein, 10 aa, synthetic construct
  REGION 1..10: note = VLCDR3 NS1G7
ASYDTNSGSV

SEQ ID NO: 363 - protein, 244 aa, synthetic construct
  REGION 1..244: note = CA2 amino acid sequence
QVQLQESGPSLVKPSQTLSLTCTVSGFSLSSVAVNWVRQAPGKVPEWLGGIISNGGTGYNPALKSRLSITRDTSKSQVSLALTHVTTEDTAVYYCGRGVEWEGSMDYLGPGLLVTVSSEGKSSGSGSETKVDDQSVLTQPSSVSGFLGQRVTITCSGSSSNVGAGSYVGWYQQVPGSGLRILIYGATKRASGLPDRFSGSRSGNTATLTISSLQAEDEADYYCVSYQTDFTLVFGSGTRLTVLG

SEQ ID NO: 364 - protein, 245 aa, synthetic construct
  REGION 1..245: note = CB7 amino acid sequence
QVQLQESGPSLVKPSQTLSLTCTVSGFSLTNYRVGWVRQAPGKALEWVSNIRSGGTTWYNPALKSRLSITADTSKSQVSLSLSSVTTEDTAVYYCARDSSGDLYAYDYWGPGLLVTVSSEGKSSGASGESKVDDQAVLTQPSSVSRSLGQSVSMTCSGSSSNVGYGNYMAWFQQVPGSAPKLLIYGATSRASGVPDRFSGSRSGNTATLTISSLQAEDEADYYCASYDSTSGGVFGSGTRLTVLG

SEQ ID NO: 365 - protein, 244 aa, synthetic construct
  REGION 1..244: note = CC7 amino acid sequence
QVQLQESGPSLVKPSQTLSLTCTVSGFSLTSNAVIWVRQAPGKAPEWVALIDVDGDAAYDPALKSRLSITRDTSKSQVSLSLRSVTTEDTAVYYCARDYGSWGYVSDIDYWGPGLLVTVSSEGKSSGASGESKVDDRVVRTQPSSVSGSLGQRVSITCSGSYITGSSVGWFQQVPGSGLKTVIYDNNDRPSGVPDRFSGSKSGDTATLTISSLQAEDEADYYCASYDTSNIGLFGSGTRLTVLG

SEQ ID NO: 366 - protein, 250 aa, synthetic construct
  REGION 1..250: note = CE2/E1B8 amino acid sequence
QVQLQESGPSLVKPSQTLSLTCTVSGFSLTNYPVGWVRQAPGKALEWIGNIENDGSANYASALKSRLSITRDTSKNQVSLSLSSATTEDTAVYYCGREFGGSDGYTYFVDIDYWGPGLLVTVSSEGKSSGASGESKVDDQAVLTQPSSVSKSLGQSVSITCSGSSSNVGYGNYVSWFQQVPGSAPKILIYGATSRASGVPDRFSGSRSGNTATLTITSLQAEDEADYYCASYDGSSSGVFGSGTRLTVLG

SEQ ID NO: 367 - protein, 250 aa, synthetic construct
  REGION 1..250: note = CE3 amino acid sequence
QVRLQESGPSLVKPSQTLSVTCTVSGFSLISNAVGWVRQAPGKVPESLAGCSSDGKCYYNSALKSRLDITRDTSKNQISLSLSSVTTDDAAVYYCTRGYYPVYGYDYLGTIDYWGPGLLVTVSSEGKSSGASGESKVDDQAVLTQPSSVSGSLGQRVSITCSGSSSNVGRNDVAWFQQLPGSGLRTIIYGTTSRPSGIPDRFSGSKSGVTATLTIDSLQAEDEADYFCASGDSSAINDIFGSGTRLTVLG

SEQ ID NO: 368 - protein, 234 aa, synthetic construct
  REGION 1..234: note = CA4 amino acid sequence
QVQLQESGPSLVKPSQTLSLTCTVSGFSLTSYSVYWVRQAPGQALEWISIMYASGRVDYNPALKSRLSITRDTSKSQFSLSLSSVTTEDTAVYYCTRGIENWGPGLLVTVSSEGKSSGASGESKVDDDIQVTQSPSSLSASLTERVSITCRTSQSVNNYLSWYQQKPGQAPKLLIYYATRLYTDVPSRFSGSGSGTDYTLTITSLEADDTATYYCLQYDSTPLAFGGGTNVEIK

SEQ ID NO: 369 - protein, 249 aa, synthetic construct
  REGION 1..249: note = CB2 amino acid sequence
QVQLQESGPSLVKPSQTLSLTCTVSGFSLSTNSVGWVRQAPGKAPEWVAGIDTDGEEGFNPVLKSRLSITRDTSKSQVSLSLSNVTSEDTAVYYCGRSYRTDGLAYGYVQAIDYWGPGLLVTISSEGKSSGASGESKVDDQSVLTQPSSVSGSPGQTVSITCSGSYIGSSGVGWFQQLPGSGLRTIIVASDGRPSGVPDRFSMSKSGNTATLTISSLQAEDEADYFCGSSDRTQYTGLFGSGTRLTVLG

SEQ ID NO: 370 - protein, 244 aa, synthetic construct; identical to SEQ ID NO: 363
  REGION 1..244: note = CB3 amino acid sequence

SEQ ID NO: 371 - protein, 249 aa, synthetic construct
  REGION 1..249: note = CA9 amino acid sequence
QVQLQESGPSLVKPSQTLSLTCTVSGFSLTSNSVGWVRQAPGKAPEWVAGIDTDGEEGYNPALNSRLSITRDTSKSQVSLSLSSVTSEDTAVYYCGRSYRSDGLAYGYVQAIDYWGPGLLVTVSSEGKSSGASGESKVDDQAVLTQPASVSGSLGQRVSITCSGSFIGISSVGWFQQLPGSGLRTIIVASDGRPSGVPDRFSMSKSGNTATLTISSLQAEDEADYFCGSSDRTQYTGVFGSGTRLTVLG

SEQ ID NO: 372 - protein, 251 aa, synthetic construct
  REGION 1..251: note = CA12 amino acid sequence
QVQLQGSGPSLVKPSQTLSLTCTVSGFSFDSYYVGWVRQAPGKALEWLGNIYSTGRAFYNPALKSRLSITRDTSKSQVSLSVSSVTIEDTALYYCVRGSYYHGGGNGMVDFFDYWSPGLLVTVSSEGKSSGASGESKVDDQVVRTQPSSVSGSLGQRVSITCSGSSSNVGYGNYVGWFQQVPGSAPKLLIYAATSRASGVPDRFSGSRSGNTATLTIDSLQAEDEADYYCSSYQRGNTGVFGSGTRLTVLG

SEQ ID NO: 373 - protein, 240 aa, synthetic construct
  REGION 1..240: note = MD9 amino acid sequence
QVRLQESGPSLVKSSQTLSLTCTVSGFSLTRESIAWVRQAPGKVPEWLGGVGIDGTSYYSPALKSRLSITRDTSKSQASLSLSSVATEDTAMYYCARNYIDFEYWGPGLLVTVSSEGKSSGASGESKVDDQAVLTQLSSVSGSLGQRISITCSGSSSNVGIYDVSWFQQLPGSGLRTVIYGTNNRPSGVPDRFSGSRSGNTATLTISSLQSEDEAIYYCAAGDSSTIAVFGSGTRLTVLG

SEQ ID NO: 374 - protein, 252 aa, synthetic construct
  REGION 1..252: note = NS1G7 amino acid sequence
QVQLQESGPSLVKPSQTLSLTCTVSGFSLTSYGVGWVRQAPGKTLEWISSISSGGTTFYNPALKSRLSITRDTSESQVSLSLSSVTTEDTAVYYCTRDVHIYYNDYGAAYGDRDYWGPGLLVTVSSEGKSSGASGESKVDDQAVVTQPPSVSGSPGQRVSITCSGSSSNIGGGNYVSWYQQLPGSGLRTLIYGTTSRASGVPDRFSGSGSGNTATLTISSLQAEDEADYYCASYDTNSGSVFGSGTRLTVLG

SEQ ID NO: 375 - protein, 21 aa, Homo sapiens
GGGQVEVKSEKLDFKDRVQSK

SEQ ID NO: 376 - protein, 12 aa, Homo sapiens
CGSLGNIHHKPG

SEQ ID NO: 377 - protein, 9 aa, Homo sapiens
SLDNITHVP

SEQ ID NO: 378 - protein, 9 aa, Homo sapiens
VQIVYKPVD

SEQ ID NO: 379 - protein, 10 aa, Homo sapiens
KKIETHKLTF

SEQ ID NO: 380 - DNA, 753 bp, synthetic construct
  misc_feature 1..753: note = S1D12 nucleotide sequence
  source 1..753: mol_type = other DNA
caggtgcagctgcaggagtcgggacccagcctggtgaagccctcacagaccctctccctcacctgcacggtctctggattctcattgaacaacaatgctgtaggctgggtccgccaggctccaggaaaggtgccggagtcgcttgtgggttgtagcagtgatggaacgtgttactataattcggccctgaaatcccggctcgacatcaccagggacacctccaagaaccagatctccctgtcactgagcagcgttacaactgacgacgcggccgtgtactattgtacaagaggccattatagtatttatggttatgactatcttggcactatcgactactggggcccaggactcctggtcaccgtctcctcagaaggtaaatcttctggcgcgtctggcgagtctaaagtggatgaccaggctgtgctgactcagccgtcctccgtgtccgggtccctgggccagagggtctccatcacctgctctggaagcagcagcaacgtcgggggtggtaatagtgtgggctggtaccaacacctcccaggctcaggcctcaaaaccatcatctatgatactaacagtcgaccctcgggggtcccggaccgattctctggctccaggtctggcaacacggccaccctaaccatcaactcgctccaggctgaggacgagggtgattattactgtgtaacgggtgacagcactactcatgatgatcttgtcggcagcgggaccaggctgaccgtcctgggg

SEQ ID NO: 381 - DNA, 747 bp, synthetic construct
  misc_feature 1..747: note = S1G2 nucleotide sequence
  source 1..747: mol_type = other DNA
caggtgcagctgcaggagtcgggacccagcctggtgaagccctcacagaccctctccctcacctgcacggtctctggattctcattaaccagcaattctgtgggctgggtccgacaggctccaggaaaggcgccggagtgggttgctggtatagatactgatggagaagaaggctataatccagcccttaactcccggctcagcatcaccagggacacctccaagagtcaagtctctttgtcattgagcagcgtgacaagtgaggacacggccgtgtactactgtggaagaagttatagggctgatggtcttgcttacggttatgtccaagccatcgactactggggcccaggactcctggtcaccgtctcctcagaaggtaaatcttctggcgcgtctggcgagtctaaagtggatgaccaggccgtggtgactcagccgtcctccgtgtctgggtccctgggccagagggtctccatcacctgctctggaagcttcatcggtattagtagtgtaggctggttccaacagctcccaggatcgggcctcagaaccatcatcgtggcgagtgacggtcgaccctcaggggtccccgaccgattctctatgtccaaatcgggcaacacagccaccctgaccatcagctcgctccaggctgaggacgaggccgattatttctgtggaagtagtgataggactccttatactggggtcttcggcagcgggaccaggctgaccgtcctgggt

SEQ ID NO: 382 - DNA, 705 bp, synthetic construct
  misc_feature 1..705: note = CA4 nucleotide sequence
  source 1..705: mol_type = other DNA
caggttcagctgcaggagtcgggacccagcctggtgaagccctcacagaccctctccctcacctgcacggtctctggattctcattaaccagctattccgtatactgggtccgccaggctccaggccaggcactggagtggattagtattatgtatgctagtggaagagtagactataacccggccctgaaatcccggctcagcatcaccagggacacctccaagagtcaattctccctgtcattgagcagcgtgacaactgaggacacggccgtctactactgtacaagaggaatcgaaaactggggccccggactcctggtcaccgtctcctcagaaggtaaatcttctggcgcgtctggcgagtctaaagtggatgacgacatccaggtgacccagtctccgtcctccctgtctgcatctctaacagagagagtctccatcacttgccggaccagtcagagcgttaacaattacttaagctggtatcagcagaaaccagggcaagctcctaagctcctgatctattatgcaaccagattgtacaccgatgtcccatcccggttcagtggcagtggatctgggacagattacaccctcaccatcaccagcctggaggcggacgacactgcaacttattactgtctacaatatgatagtacacctcttgcattcggcggtgggaccaacgtggaaatcaaacgg

SEQ ID NO: 383 - DNA, 750 bp, synthetic construct
  misc_feature 1..750: note = CE2 nucleotide sequence
  source 1..750: mol_type = other DNA
caggtgcagctgcaggagtcgggacccagcctggtgaagccctcacagaccctctccctcacctgcacggtctctggattctcattaaccaactatcctgtaggctgggtccgccaggctccaggaaaggcactggagtggattggtaacatagaaaatgatggaagtgcgaactatgcctcggccctgaaatcccgactcagcatcaccagggacacctccaagaaccaagtctccctgtcactgagcagcgcgacaactgaggacacggccgtttactactgtggaagagaattcggtgggagtgatggttatacttatttcgttgatatcgactactggggcccaggactcctggtcaccgtctcctcagaaggtaaatcttctggcgcgtctggcgagtctaaagtggatgaccaggctgtgctgactcagccgtcctccgtgtccaagtccctgggccagagtgtctccatcacctgctctggaagcagcagcaacgttggatatggtaattatgtgagctggttccaacaggtcccaggatcagcccccaaaatcctcatctatggtgcaaccagtcgagcctcgggggtccccgaccgattctccggctccaggtctggcaacacagcgactctgaccatcacctcgctccaggctgaggacgaggccgattattactgtgcatcttatgacggcagtagcagtggtgttttcggcagcgggaccaggctgaccgtcctgggt

SEQ ID NO: 384 - DNA, 750 bp, synthetic construct
  misc_feature 1..750: note = CE3 nucleotide sequence
  source 1..750: mol_type = other DNA
caggtgcgactgcaggagtcgggacccagcctggtgaagccctcacagaccctctccgtcacctgcacggtctctggattctcattgatcagcaatgctgtaggctgggtccgccaggctccaggaaaggtgccggagtcgcttgctggttgtagcagtgatggaaagtgttactataactcggccctgaaatcccggctcgacatcaccagggacacctcgaagaaccagatctccctgtcactgagcagcgtcacaactgacgacgcggccgtgtactactgtacaagaggctattatcctgtttatggttatgactatcttggcactatcgactactggggccccggactcctggtcaccgtctcctcagaaggtaaatcttctggcgcgtctggcgagtctaaagtggatgaccaagctgtgctgactcaaccgtcctccgtgtctgggtccctgggccagagggtctccatcacctgctctggaagcagcagcaacgttggtagaaatgatgtagcctggttccaacaactcccaggatcaggcctcagaaccatcatctatggtactaccagtcgaccctcaggtatcccggaccgattctccggctccaagtctggcgttacggccaccctgaccatcgactcgctccaggctgaggacgaggccgattatttctgtgcctctggtgacagtagtgccattaatgatattttcggcagcgggaccaggctgaccgtcctgggt

SEQ ID NO: 385 - DNA, 735 bp, synthetic construct
  misc_feature 1..735: note = CB7 nucleotide sequence
  source 1..735: mol_type = other DNA
caggtgcagctgcaggagtcgggacccagcctggtgaagccctcacagaccctctccctcacctgcacggtctctggattctcattaaccaactatcgtgtaggttgggtccgccaggctccaggaaaggcactggagtgggttagtaacatacggagtggtggaactacatggtataacccggccctgaaatcccggctcagcatcaccgcggacacctccaagagccaagtctccctgtcactgagcagcgtgacaactgaggacacggccgtatattattgtgcaagagattcctctggtgatctttatgcgtatgattactggggcccaggactcctggtcaccgtctcctcagaaggtaaatcttctggcgcgtctggcgagtctaaagtggatgaccaggccgtgctgactcagccgtcctccgtgtccaggtccctgggccagagtgtctccatgacctgctctggaagcagcagcaacgttggatatggtaattatatggcctggttccaacaggttccaggatcagcccccaaactcctcatctatggtgcaaccagtcgagcctcgggggtccccgaccgattctccggctccaggtctggcaacacagcgactctgaccatcagctcgctccaggctgaggacgaggccgattactactgtgcatcttatgacagcactagcgggggtgtcttcggcagcgggaccaggctgaccgtcctgggt

SEQ ID NO: 386 - DNA, 732 bp, synthetic construct
  misc_feature 1..732: note = CC7 nucleotide sequence
  source 1..732: mol_type = other DNA
caggtgcagctgcaggagtcgggacccagcctggtgaagccctcacagaccctctccctcacctgcacggtctctggattctcactaaccagcaatgctgtgatctgggtccggcaggctccaggaaaggcgccggagtgggttgctttgatagatgttgatggagatgcagcctatgacccagcccttaagtcccgcctcagcatcaccagggacacctccaagagtcaagtctccctttcactgcgcagcgtgacaactgaggacacggccgtgtactactgtgcaagagactatggtagttggggttatgtttccgacatcgactactggggcccaggactcctggtcaccgtctcctcagaaggtaaatcttctggcgcgtctggcgagtctaaagtggatgacagggtcgtgcggactcaaccgtcctccgtgtctgggtccctgggccagagggtctccatcacctgctctggaagctacatcactggtagttctgtaggctggttccaacaggtcccaggatcgggcctcaaaaccgtcatctatgacaataacgatcgaccctcaggggtccccgaccgattctctggctccaagtcgggcgacacagccaccctgaccatcagctcgctccaggctgaggacgaggccgattattactgtgcatcttatgacaccagtaacattggtcttttcggcagcgggaccaggctgaccgtcctgggt

SEQ ID NO: 387 - DNA, 723 bp, synthetic construct
  misc_feature 1..723: note = 412E10 nucleotide sequence
  source 1..723: mol_type = other DNA
caggtgcagctgcaggagtcgggacccagcctggtgaagccctcacagaccctctccctcacctgcacggtctctggcttctctgtaataagcgattctgtagcctgggtccgccaggctccaggaaaagtgccggagtggcttggtgctagcggcagttctggaaacaaatactataacccggccctaaaatcccggctcagcatcaccagggacacctccaagagccaagtctccctgtcactgagcagcgtgacaactgaggatacggccgtgtactactgtgcgagaggtattatcgccggtgtagatgtctggggccgaggactcctggtcaccgtctcctcagaaggtaaatcttctggcgcgtctggcgagtctaaagtggatgaccaggctgtgctgactcagccgtcctccgtgtctgggtccctgggccagagggtctccatcacctgctctggaagcagcagcaacgttggatatggtaattatgtgggctggtaccaacaggtcccaggatcagcccccaaactcctcatctatggtacagccattcgagcctcgggggtccccgaccgattctccggctccaggtctggggacacagccacccttaccatcacctcgctccaggctgaggacgaggccgattactactgtgcatcttatcagagtaattacgcttttttcggcagcgggaccaggctgaccgtcctgggt

SEQ ID NO: 388 - DNA, 747 bp, synthetic construct
  misc_feature 1..747: note = S1B1 nucleotide sequence
  source 1..747: mol_type = other DNA
caggtgcagctgcaggagtcgggacccagcctggtgaagccctcacagaccctctccctcacctgcacggtctctggattctcattgtccagcaattctgtgggctgggtccgacaggctccaggaaaggcgccggagtgggttgctggtatagatactgatggagaagaaggctataatccagcccttaactcccggctcagcatcaccagggacacctccaagagtcaagtctctttgtcattgagcagcgtgacaagtgaggacacggccgtgtactactgtgtaagaagttataggactgatggtcttgcttacggttatgtccaagccatcgactactggggcccaggactcctggtcaccgtctcctcagaaggtaaatcttctggcgcgtctggcgagtctaaagtggatgaccaggctgtgctgactcagccgtcctccgtgtctgggtccctgggccagagggtctccatcacctgctctggaagcttcatcggtattagtagtgtaggctggttccaacagctcccaggatcgggcctcagaaccgtcatcgttgcgagtgacggtcgaccctcaggggtccccgaccgattctctaactccaaatcgggcaacacagccaccctgaccatcagctcgctccaggctgaggacgaggccgattatttctgtggaagtagtgataggactcaatatactggagttttcggcagcgggaccaggctgaccgtccttggt

SEQ ID NO: 389 - DNA, 747 bp, synthetic construct
  misc_feature 1..747: note = S1D9 nucleotide sequence
  source 1..747: mol_type = other DNA
caggtgcagctgcaggagtcgggacccagcctggtgaagccctcacagaccctctccctcacctgcacggtctctggattctcattaaccagcaattctgtgggctgggtccgacaggctccaggaaaggcgccggagtgggttgctggtatagatagtgatggagaagaaggctataatccagcccttaactcccggctcagcatcaccagggacacctccaagaatcaagtctctttgtcattgagcagagtgacaagtgaggacacggccgtttactactgtggaagaacttataggactgatggttatgcttacggttatgtccaagccatcgactactggggcccaggactcctggtcaccgtctcctcagaaggtaaatcttctggcgcgtctggcgagtctaaagtggatgaccgggtcatgctgactcagccaccctccgtgtccgggtccccgggccagacggtatccatcacctgctctggaagcttcatcggtattagtagtgtaggctggttccaacagctcccaggatcgggcctcagaaccgtcatttttgcgagtgacggtcgaccctcaggggtccccgatcgattctctaactccaaatcgggcaacacagccaccctgaccatcagctcgctccaggctgaggacgaggccgattatttctgtggaagtagtgataggactcaatatactggagttttcggcagcgggaccaggctgaccgtcctgagt

SEQ ID NO: 390 - DNA, 747 bp, synthetic construct
  misc_feature 1..747: note = S1F4 nucleotide sequence
  source 1..747: mol_type = other DNA
caggtgcagttgcaagagtcgggacccagcctggtgaagccctcacagaccctctccctcacctgcacggtctctggattctcattaagtagcaattctgtcggctgggtccgacaggctccaggaaaggcgccggagtgggttgctggtatagatactgatggagaagaaggctataatccagcccttaactcccggctcagcatcaccagggacacctccaagagtcaagtctcattgtcattgagcagcgttacaagtgaggacacggccgtgtactactgtgtaagaagttatagagctgatggtcttgcttacggttatgtccaagccatcgactactggggcccaggactcctgctcaccatctcctcagaaggtaaatcttctggcgcgtctggcgagtctaaagtggatgaccaggccgtggtgactcagccgtcctccgtgtctgggtccctgggccagagggtctccatcacctgctctggaagcttcatcggtattagtagtgtaggctggttccaacagctcccaggatcgggcctcagaaccgtcatcgtggcgagtgacggtcgaccctcaggggtccccgaccgattctctaactccaaatcgggcaacacagccaccctgaccatcagctcgctccaggctgaggacgaggccgattatttctgtggaagtagtgataggactcaatatactggagttttcggcagcgggaccaggctgaccgtcctgggt

SEQ ID NO: 391 - DNA, 747 bp, synthetic construct
  misc_feature 1..747: note = S1G10 nucleotide sequence
  source 1..747: mol_type = other DNA
caggttcagttgcaggagtcgggacccagcctggtgaagccctcacagaccctctccctcacctgcacggtctctggattctcattaaccagcaattctgtgggctgggtccgacaggctccaggaaaggcgccggagtgggttgctggtatagatactgatggagaagaaggctataatccagcccttaactcccggctcagcatcaccagggacacctccaagagtcaagtctcattgtcattgagcagcgtgacaagtgaggacacggccgtgtactactgtggaagaagttatagagctgatggtcttgcttacggttatgtccaagccatcgactactggggcccaggactcctggtcaccgtctcctcagaaggtaaatcttctggcgcgtctggcgagtctaaagtggatgaccaggctgtgctgactcagccgtcctccatgtccgggtccctgggccagagggtctccatcacctgttctggaagcttcatcggtattagtagtgtaggctggttccaacagctcccaggatcgggcctcagaaccatcatcgtggcgagtgacggtcgaccctcaggggtccccgaccgattctctatgtccaaatcgggcaacacagccaccctgaccatcagctcgctccaggctgaggacgaggccgattatttctgtggaagtagtgataggactcaatatactggagttttcggcagcgggaccaggctgaccgtcctgggt

SEQ ID NO: 392 - DNA, 750 bp, synthetic construct
  misc_feature 1..750: note = S2C6 nucleotide sequence
  source 1..750: mol_type = other DNA
caggtgcagctgcaggagtcgggacccagcctggtgaagccctcacagaccctctccctcacctgcacggtctctggattctcattaatcagcaattctgtgggctgggtccgacaggctccaggaaaggcgccggagtgggttgctggtatagatactgatggagaagaaggctataatccagcccttaagtcccaatatgcagctagtgatccggacacctccaagagtcaagtctccttgtcattgagcagcgtgacaagtgaggacacggccgtgtactactgtggaagaacttataggactgatggttttgcttatggttatgtccaagccatcgactactggggcccaggactcctgctcactatctcctcagaaggtaaatcttctggcgcgtctggcgagtctaaagtggatgaccaggctgtgctgactcagccgtcctccgtgtctgggtccctgggccagagggtctccatcacctgctctggaagctttattggtattagtagtgtaggctggttccaacagctcccaggatcgggcctcagaaccatcatcgtggcgagtgacggtcgaccctcaggggtccccgaccgattctctatgtccaaatcgggcaacacagccaccctgaccatcagctcgctccaggctgaggacgaggccgattatttctgtggaagtagtgataggactcaatatactggagttttcggcagcgggaccaggctgaccgtcctgggt

SEQ ID NO: 393 - DNA, 720 bp, synthetic construct
  misc_feature 1..720: note = MD9 nucleotide sequence
  source 1..720: mol_type = other DNA
caggtgcggctgcaggagtcgggacccagcctggtgaagtcctcacagaccctctccctcacctgcacggtctctggattctcattaaccagggaatctatagcctgggtccgccaggctccaggaaaggtgccggagtggcttggtggtgtaggcattgatggaacctcatactatagcccggccctgaaatcccggctcagtatcacgagggacacctccaagagccaagcctccctgtcactgagcagcgtggcaactgaggacacggccatgtattactgtgcacgtaattatattgatttcgagtactggggcccaggactcctggtcaccgtctcctcagaaggtaaatcttctggcgcgtctggcgagtctaaagtggatgaccaggctgtgctgactcaactgtcctccgtatctgggtccctgggccagaggatctccatcacctgctctggaagcagcagcaacgttggtatatatgatgtgtcttggttccaacaactcccaggatcaggcctcagaaccgtcatctatggtactaacaatcgaccctcgggtgtcccggaccgattctccggctccaggtctggcaacacggccaccctgactatcagctctctccagtctgaggacgaggccatttattactgtgctgctggtgacagcagtactattgctgttttcggcagcgggaccaggctgaccgtcctgggt

SEQ ID NO: 394 - DNA, 726 bp, synthetic construct
  misc_feature 1..726: note = 412B9 nucleotide sequence
  source 1..726: mol_type = other DNA
caggtgcagctgcaggagtcgggacccagcctggtgaagccctcacagaccctctccctcacctgcacggtctctggattctctgtaataagcgattctgtagcctgggtccgccaggctccaggaaaagtgccggagtggcttggtgctagcggcagttctggaaacaaatactataacccggccctaaaatcccggctcagcatcaccagggacacctccaagagccaagtctccctgtcactgagcagcgtgacaactgaggatacggccgtgtactactgtgcgagaggtattatcgccggtgtagatgtctggggccgaggactcctggtctccgtctcctcagaaggtaaatcttctggcgcgtctggcgagtctaaagtggatgaccaggctgtgctgactcagccgtcctccgtgtctggggccctgggccagagggtctccatcacctgctctggaagcagcagcaacgttggatatggtgattatgtgggctggtaccaacaggtcccaggatcagcccccaaactcctcatctatggtgcagccagtcgagcctcgggggtccccgaccgattctctggctccaggtctggcaacacagcgactctgaccatcagctcgctccaggctgaggacgaggccgattattactgtgcatcttatgacagcagtgacggtggtgttttcggcagcgggaccaggctgaccgtcctgggt

SEQ ID NO: 395 - DNA, 729 bp, synthetic construct
  misc_feature 1..729: note = 412E6 nucleotide sequence
  source 1..729: mol_type = other DNA
caggtgcagctgcaggagtcgggacccagcctggtgaagccctcagagaccctctccctcacctgcacggtctctggattctcattaacaaactatggtgtgggctgggtccgccaggctccaggaaaggcactggagtggcttggtaacatatatagtggtgggtctacatactataacccggccctgaaatcccgactcagcatcaccagggacacctccaagagccaagtctccctgtcactgaacagcgtgacacttgaggacacggccgtttattactgtggaagaggaggtgttgggagtgtcgacgtctggggcccaggactcctggtcaccgtctcctcagaaggtaaatcttctggcgcgtctggcgagtctaaagtggatgaccaggctgtgctgactcagccgccctccgtgtccggttccccaggccagagggtctccatcacctgctctggaggcaggaataacatcgggcgtggtacctttgtggactggtaccagcaactcccaggatcaggcctcaaaaccgtcatctatggtactgaccgtcgaccaccgggggtcccggaccgattctccggctccaagactggcaacgcggccaccctgaccatcacctccctccaggctgaggacgaggccgattattggtgtgctacttatgattacagtaatgatatgattattctcggcagcgggaccaggctgaccgtcctgggt

SEQ ID NO: 396 - DNA, 714 bp, synthetic construct
  misc_feature 1..714: note = 412G11 nucleotide sequence
  source 1..714: mol_type = other DNA
caggtgcggctgcaggagtcgggacccagcctggtgaagccctcacagaccctctccctcacctgtacggtctctggattctcattaaccagctatgctttaggctgggtccgccaggctccaggaagggctccggagtggattggtaacatatggaggggtggacgaatagaatataacccggccctgaaatcccggctcagcatcactagggacacctccaagagccaagtctcgctgtcactgagcagcgtgacaactgaggatacggccgtgtactactgttcaagaagtggcggcgactggggcccaggactcctggtcaccgtctcctcagaaggtaaatcttctggcgcgtctggcgagtctaaagtggatgaccaggctgtgctgactcagccgtcctccgtgtctgggtccctgggccagagggtctccatcacctgctctggaagcagcagcaacgttggatatggtaattatgtgggctggtaccaacaggtcccaggatcagcccccaanctcctcatctatggtgcaaccagtcgagcctcgggggtccccgaccgattctccggctccaggtctgagaacacagccaccctgaccatcagctccctccaggctgaggacgaggccgattattactgtgcgtcttatgataggagtgagagtgttgtgttcggcagcgggaccagactgaccgtcctgggt

SEQ ID NO: 397 - DNA, 732 bp, synthetic construct
  misc_feature 1..732: note = CA2 nucleotide sequence
  source 1..732: mol_type = other DNA
caggttcagctgcaggagtcgggacccagcctggtgaagccctcacagaccctctccctcacctgcacggtctctggattctcattaagcagcgtcgctgtaaactgggtccgccaggctccaggaaaggtgccggagtggcttggtggcattattagtaatggaggcacaggctataatccggccctgaaatctcggctgagcatcaccagggacacctccaagagccaagtctccctggcactgacccacgtgacaactgaggacacggccgtgtactactgtggaaggggagttgaatgggagggctctatggactacttgggcccaggactcctggtcaccgtctcctcagaaggtaaatcttctggctctggctctgagactaaagtggatgaccagtctgtgctgactcagccgtcctccgtgtccgggttcctgggccagagggtcaccatcacctgctctggaagcagcagcaacgttggagctggtagttatgtgggctggtaccagcaggtcccaggatcgggcctcagaatcctcatctatggtgcaaccaagcgagcctcgggactccccgaccgattctccggctccaggtctgggaacacagccaccctgaccatcagctcgctccaggctgaggacgaggccgattattactgcgtatcttatcagactgattttactttagttttcggcagcgggaccaggctgaccgtcctaggt

SEQ ID NO: 398 - DNA, 747 bp, synthetic construct
  misc_feature 1..747: note = CA9 nucleotide sequence
  source 1..747: mol_type = other DNA
caggtgcagcttcaggagtcgggacccagcctggtgaagccctcacagaccctctccctcacctgcacggtctctggattctcattaaccagcaattctgtgggctgggtccgacaggctccaggaaaggcgccggagtgggttgctggtatagatactgatggagaagaaggctataatccagcccttaactcccggctcagcatcaccagggacacctccaagagtcaagtctctttgtcattgagcagcgtgacaagtgaggacacggccgtgtactactgtggaagaagttataggagtgatggtcttgcttacggttatgtccaagccatcgactactggggcccaggactcctggtcaccgtctcctcagaaggtaaatcttctggcgcgtctggcgagtctaaagtggatgaccaagctgtgctgactcagccggcctccgtgtctgggtccctgggccagagggtctccatcacctgttctggaagcttcatcggtattagtagtgtaggctggttccaacagctcccaggatcgggcctcagaaccatcatcgtggcgagtgacggtcgaccctcaggggtccccgaccgattctctatgtccaaatcgggcaacacagccaccctgaccatcagctcgctccaggctgaggacgaggccgattatttctgtggaagtagtgataggactcaatatactggagttttcggcagcgggaccaggctgaccgtcctgggt

SEQ ID NO: 399 - DNA, 753 bp, synthetic construct
  misc_feature 1..753: note = CA12 nucleotide sequence
  source 1..753: mol_type = other DNA
caggtgcagctgcaggggtcgggacccagcctggtgaagccctcacagaccctctccctcacctgcacggtctctggattctcttttgacagctattatgtaggctgggtccgccaggctccaggaaaggcactggagtggcttggtaatatatatagtactggaagggcattctataacccggccctgaaatcccggctcagcatcaccagggacacctccaagagccaagtctccctatcagtgagcagcgtgacaattgaggacacggccctgtactactgtgtcagaggctcgtattatcacggtggtggcaatgggatggtcgactttttcgactactggagcccaggactcctggtcaccgtctcctcagaaggtaaatcttctggcgcgtctggcgagtctaaagtggatgaccaggtcgtgcggactcagccgtcctccgtgtctgggtccctgggccagagggtctccatcacctgctctggaagcagcagcaatgttggatatggtaattatgtgggctggttccaacaggtgccagggtcagcccccaaactcctcatctatgctgcaaccagtcgagcctcgggggtccccgaccgattctccggctccaggtctgggaatacagccaccctgaccatcgactcgctccaggctgaggacgaggccgattattactgttcatcttatcaacgcggtaacactggtgttttcggcagcgggaccaggctgaccgtcctgggt

SEQ ID NO: 400 - DNA, 747 bp, synthetic construct
  misc_feature 1..747: note = CB2 nucleotide sequence
  source 1..747: mol_type = other DNA
caggttcagcttcaggagtcgggacccagcctggtgaagccctcacagaccctctccctcacctgcacggtctctggattctcattaagcactaattctgtgggctgggtccgacaggctccaggaaaggcgccggagtgggttgctggtatagatactgatggagaagaaggctttaatccagtccttaagtcccggctcagcatcaccagggacacctccaagagtcaagtctctttgtcattgagcaacgtgacaagtgaagacacggccgtgtactactgtggaagaagttataggactgatggtcttgcttacggttatgtccaagccatcgactactggggcccaggactcctggtcactatctcctcagaaggtaaatcttctggcgcgtctggcgagtctaaagtggatgaccagtctgtgctgactcagccgtcctccgtgtccgggtccccgggccagacagtctccatcacctgctctggaagctatatcggtagtagtggtgtaggctggttccaacagctcccaggatcgggcctcagaaccatcatcgtggcgagtgacggtcgaccctcaggggtccccgaccgattctctatgtccaaatcgggcaacacagccaccctgaccatcagctcgctccaggctgaggacgaggccgattatttctgtggaagtagtgataggactcaatatactggacttttcggcagcgggaccaggctgaccgtcctgggt

SEQ ID NO: 401 - DNA, 732 bp, synthetic construct; identical to SEQ ID NO: 397
  misc_feature 1..732: note = CB3 nucleotide sequence
  source 1..732: mol_type = other DNA

SEQ ID NO: 402 - DNA, 735 bp, synthetic construct; identical to SEQ ID NO: 385
  misc_feature 1..735: note = CB7 nucleotide sequence
  source 1..735: mol_type = other DNA

SEQ ID NO: 403 - DNA, 732 bp, synthetic construct; identical to SEQ ID NO: 386
  misc_feature 1..732: note = CC7 nucleotide sequence
  source 1..732: mol_type = other DNA

SEQ ID NO: 404 - DNA, 741 bp, synthetic construct
  misc_feature 1..741: note = 3aA6 nucleotide sequence
  source 1..741: mol_type = other DNA
caggtgcggctgcaggagtcgggatccagtctggtgaagccctcacagaccctctccctcgtctgcacggtctctggattcccattaaccagcaatgctgtaggctgggtccgccaggctccaggaaaggcgccggagtggctaggtctcatagatattgatggagacacagcctataacccagcccttgagtcccggctcagcatcaccagggacacctccaagagtcaagtctccctgtcactgagcagcgtggcaattgaggacacggccgtgtactattgtgctcgtcattatgataaatggggttatgctgattcgatcgactactggggcccaggactcctggtcaccgtctcctcagaaggtaaatcttctggcgcgtctggcgagtctaaagtggatgaccaggccctgctgactcagccgtcctccgtgtttggttccctgggccagagggtctccatcacctgctctggaagcagcagcaacgttggatatggtgattatgtaggctggtaccaacaggtcccaggatcagcccccaaactcctcatctatgatgcaaccactcgagcctcgggggtccccgaccgattctccggctccaggtctgggaacacagccaccctgaccatcagctcgctccaggctgaggacgaggccgattattactgtgcatcttatcagaatgaaagaagtggtgttttcggcagcgggaccaggctgaccgtcctgggt

SEQ ID NO: 405 - DNA, 732 bp, synthetic construct
  misc_feature 1..732: note = 3aD6 nucleotide sequence
  source 1..732: mol_type = other DNA
caggtgcagctgcaggagtcgggacccagcctggtgaagccctcacagaccctctccctcacctgcacggtctctggattctcactaaccagcaatgctgtgatctgggtccggcaggctccaggaaaggcgccggagtgggttgctttgatagatgttgatggagatgcagcctatgacccagcccttaagtcccgcctcagcatcaccagggacacctccaagagtcaagtctccctttcactgcgcagcgtgacaactgaggacacggccgtgtactactgtgcaagagactatggtagttggggttatgtttccgacatcgactactggggcccaggactcctggtcaccgtctcctcagaaggtaaatcttctggcgcgtctggcgagtctaaagtggatgaccaggctgtgctgactcagccgtcctccgtgtctgggtccctgggccagagggtctccatcacctgctctggaagcgacatcggtggtgctgatgtaggctggttccaacaggtcccaggatcgggcctcagaaccctcatctatgataatgacaatcgaccctcaggggtccccgaccgattctctggctccaagtcgggcaacacagccaccctgaccatcagctcgctccagcctgaggatgaggccgattatttctgtggcacttattctggtgctaactatggtatttttggcagcgggaccaggctgaccgtcctgggt

SEQ ID NO: 406 - DNA, 744 bp, synthetic construct
  misc_feature 1..744: note = 3aB7 nucleotide sequence
  source 1..744: mol_type = other DNA
caggttcagctgcaggagtcgggacccagcctggtgaagccctcacagaccctctccctcacctgcacggtctctggattctcattaactagcaatggtgtaggctgggtccgccaggctccaggaaaggtgccggagtgggttggtgataaaagcagtgctggaaagacatacggtaacccggccctgaaatcccggctcagcatcaccagggacacctccaagagccaagtctccctgtcactgagcagcgtgacaactgaggacacggccgtgtactactgtgtaagatgcagggatggtggtgtgacttatggttatgacgtcgactactggggcccaggactcctggtcaccgtctcctcagaaggtaaatcttctggcgcgtctggcgagtctaaagtggatgaccaggctgtgctgactcagccgtcctccgtgtccaagtccctgggccagagtgtctccatcacctgctctggaagcagcagcaacgttggatatggtgatgttgtgagctggttccaacagttcccaggatcagcccccaaactcctcattttcggtacaacgactcgagcctcgggggtccccgaccgattctccggctccaggtctggcaacgcagcgactctaaccatcaactctctccaggctgaggacgaggccgactattactgtgcgtcttttgatagtgatagcggtggaattgccggcagcgggaccaggctgaccgtcctgggt

SEQ ID NO: 407 - DNA, 744 bp, synthetic construct
  misc_feature 1..744: note = 3bF4 nucleotide sequence
  source 1..744: mol_type = other DNA
caggtgcagctgcaggagtcgggacccagcctggtgaagccctcacagaccctctccctcacctgcacggtctctggattctccttgagtagcaatggtgtaggctgggtccgccaggctccaggaaaggtgccggagtggcttggtgatataagcagtgttggaaaaaaatacgctaacccggccctgaaatctcggctcagcttcactagggacacctccaagagccaagtgtccctgtcactgagcagcgtgacaactgaggacacggccgtgtactattgtgtaaaatgcagggatggtggtgtgacttatggttatgatatcgactactggggcccaggactcctggtcaccgtctcctcagaaggtaaatcttctggcgcgtctggcgagtctaaagtggatgaccaggctgtgctgactcagccgtcctccgtgtctaagtccacgggccagactgtctccatcacctgctctggaagcagcagcaacgttgggttacgtaattatgtgacctggttccaacaggtcccaggatcagcccccaaactcctcatctatggtgcaaccagtcgagcctcggggatccccgaccgattctccggctccaggtctggcaacacagcgactctgatcatcagctcgctccaggctgaggacgaggccgattattactgtgcatctgctgacaccaatgacggtggtgttttcggcagcgggaccaggctgaccgtcctgggt

SEQ ID NO: 408 - DNA, 744 bp, synthetic construct
  misc_feature 1..744: note = 3aD3 nucleotide sequence
  source 1..744: mol_type = other DNA
caggtgcagctgcaggagtcgggacccagcctggtgaagccctcacagaccctctccctcacctgcacggtctctggattctcattaactagcaatggtgtaggctgggtccgccgggctccaggaaaggtgccggagtgggttggtgatataagcagtggtggaaaagtatacgggcacccggccctgaaatcccggctcagcatcaccagggacacctccaagagccaagtctccctgtcagtgagcagcgtgacaagtgaggacacggccgtgtactactgtgtaagatgcagggatggtggtgtgagttatggttatgatagcgactactggggcccaggactcctggtcaccgtctcctcagaaggtaaatcttctggcgcgtctggcgagtctaaagtggatgaccaggctgtggtgactcagccgtcctccgtgtccaagtccctgggccagagtgtctccatcacctgctctggaagcagcagcaacgttggatatggtgattatgtgggctggttccaacaggtcccaggatcagcccccaaactcctcatctatggtgtaaccgagcgagcctcgggggtccccgaccgattctccggctccaggtctggcaacacagcgactctgaccatcagctcgatccaggctgaggacgaggccgattattactgtgcatcttatgacgacagtagcggtggtattttcggcagcgggaccaggctgaccgtcctgggt

SEQ ID NO: 409 - DNA, 744 bp, synthetic construct
  misc_feature 1..744: note = 3aH6 nucleotide sequence
  source 1..744: mol_type = other DNA
caggtgcagctgcaggagtcgggacccagcctggtgaagccctcacagaccctctccctcacctgcacggtctctggattctccttgagtagcaatggtgtaggctgggtccgccaggctccaggaaaggtgccggagtggcttggtgatataagcagtgttggaaaaaaatacgctaacccggccctgaaatctcggctcagcttcactagggacacctccaagagccaagtgtccctgtcactgagcagcgtgacaactgaggacacggccgtgtactattgtgtaaaatgcagggatggtggtgtgacttatggttatgatatcgactactggggcccaggactactggtcaccgcctcctcagaaggtaaatcttctggcgcgtctggcgagtctaaagtggatgaccaggccgtggtgactcagccgtcctccgtgtctgggtccctgggccagagtgtctccatcacctgctccggaagctccggcaacgttggctatggcgattatgtgagttggttccaacaattccacggatcggcccccaaactcctcatctatggtgcaaccaatcttgcctcgggagttcccgcccgattctccggctccaggtctggcaacacggccacccttactatcagctcgctccacgctgaggacgaggccgattactattgtgcatcttatgacagcagtagcggcggtgtgttcggcagcgggaccaggctgaccgtcctgggt

SEQ ID NO: 410 - DNA, 744 bp, synthetic construct
  misc_feature 1..744: note = 3aG3 nucleotide sequence
  source 1..744: mol_type = other DNA
caggtgcagctgcaggagtcgggacccagcctggtgaagccctcacagaccctctccctcacctgcacggtctctggattctcattaagtagtaatggtgtaggctgggtccgccaggctccaggaaaggtgccggagtgggttggtgatataagtagtagtggaaaagcatacgctaatccggccctgaaatcccggctcagcatcaccagggacaccgccaagacccaagtcttcctgtcactgagcagcgtgacaactgaggacacggccgtgtactactgtgtaagatgcagggatggtggtgtaagttatggttatgatatcgactactggggcccaggactcctggtcaccgtctcctcagaaggtaaatcttctggcgcgtctggcgagtctaaagtggatgaccaggctgtgctgactcagccaccctccgtgtccgggtccccgggccagagggtatccattacctgctctggaagcagcagcaacatcgggggtggtaattatctgagctggttccaacaggtcccaggatcagcccccaaactcctcatctatggtgcaaccagtcgagcctcgggggtccccgaccgattctccggctccagatctggcaacacagcgactctgacaatcagctcgctccaggctgaggacgaggccgattattactgtgcatcttttgacaccagtagcggtggtattttcggcgccgggaccaggctgaccgtcctgggt

SEQ ID NO: 411 - DNA, 744 bp, synthetic construct
  misc_feature 1..744: note = 3bG4 nucleotide sequence
  source 1..744: mol_type = other DNA
caggtgcagcttcaggagtcgggacccagcctggtgaagccctcacagaccctctccctcacctgcacaatctctggattctcattaatcagcaatggtgtaggctgggtccgccaggctccaggaaaggtgccggagtgggttggtgatattgctagtagtggaaaggcatacagtaacccggccctgaaatcccggctcagcatcaccagggacacctccaagagccaagtctccctgtcactgaggagcgtgacaactgaggacacggccgtgtactactgtgtaagatgcagggatggtggtgtgacttatggttatgatatcgactactggggcccaggactcctggtcaccgtctcctcagaaggtaaatcttctggcgcgtctggcgagtctaaagtggatgaccaggctgtgctgactcagccgtcatccgtgtccaagtccctgggccagagtgtctccatcacctgctccggaagcactagcaacgttggaagtggtaatgatgtgagctggttccaacaggtcccaggatcagcccccaaactcctcttctacggtgcaaccaaccgagcctcgggggtccccgaccgattctccggctccaggtctggcaacacagcgactctgaccatcacctcgcttcaggctgaggacgaggccgattattactgtggatcttatgacagcaatagcggtggtattttcggcagtgggaccaggctgaccgtcctgggt

SEQ ID NO: 412 - protein, 249 aa, synthetic construct
  REGION 1..249: note = S1B1 amino acid sequence
QVQLQESGPSLVKPSQTLSLTCTVSGFSLSSNSVGWVRQAPGKAPEWVAGIDTDGEEGYNPALNSRLSITRDTSKSQVSLSLSSVTSEDTAVYYCVRSYRTDGLAYGYVQAIDYWGPGLLVTVSSEGKSSGASGESKVDDQAVLTQPSSVSGSLGQRVSITCSGSFIGISSVGWFQQLPGSGLRTVIVASDGRPSGVPDRFSNSKSGNTATLTISSLQAEDEADYFCGSSDRTQYTGVFGSGTRLTVLG

SEQ ID NO: 413 - protein, 249 aa, synthetic construct
  REGION 1..249: note = S1D9 amino acid sequence
QVQLQESGPSLVKPSQTLSLTCTVSGFSLTSNSVGWVRQAPGKAPEWVAGIDSDGEEGYNPALNSRLSITRDTSKNQVSLSLSRVTSEDTAVYYCGRTYRTDGYAYGYVQAIDYWGPGLLVTVSSEGKSSGASGESKVDDRVMLTQPPSVSGSPGQTVSITCSGSFIGISSVGWFQQLPGSGLRTVIFASDGRPSGVPDRFSNSKSGNTATLTISSLQAEDEADYFCGSSDRTQYTGVFGSGTRLTVLS

SEQ ID NO: 414 - protein, 249 aa, synthetic construct
  REGION 1..249: note = S1F4 amino acid sequence
QVQLQESGPSLVKPSQTLSLTCTVSGFSLSSNSVGWVRQAPGKAPEWVAGIDTDGEEGYNPALNSRLSITRDTSKSQVSLSLSSVTSEDTAVYYCVRSYRADGLAYGYVQAIDYWGPGLLLTISSEGKSSGASGESKVDDQAVVTQPSSVSGSLGQRVSITCSGSFIGISSVGWFQQLPGSGLRTVIVASDGRPSGVPDRFSNSKSGNTATLTISSLQAEDEADYFCGSSDRTQYTGVFGSGTRLTVLG

SEQ ID NO: 415 - protein, 249 aa, synthetic construct
  REGION 1..249: note = S1G10 amino acid sequence
QVQLQESGPSLVKPSQTLSLTCTVSGFSLTSNSVGWVRQAPGKAPEWVAGIDTDGEEGYNPALNSRLSITRDTSKSQVSLSLSSVTSEDTAVYYCGRSYRADGLAYGYVQAIDYWGPGLLVTVSSEGKSSGASGESKVDDQAVLTQPSSMSGSLGQRVSITCSGSFIGISSVGWFQQLPGSGLRTIIVASDGRPSGVPDRFSMSKSGNTATLTISSLQAEDEADYFCGSSDRTQYTGVFGSGTRLTVLG

SEQ ID NO: 416 - protein, 250 aa, synthetic construct
  REGION 1..250: note = S2C6 amino acid sequence
QVQLQESGPSLVKPSQTLSLTCTVSGFSLISNSVGWVRQAPGKAPEWVAGIDTDGEEGYNPALKSQYAASDPDTSKSQVSLSLSSVTSEDTAVYYCGRTYRTDGFAYGYVQAIDYWGPGLLLTISSEGKSSGASGESKVDDQAVLTQPSSVSGSLGQRVSITCSGSFIGISSVGWFQQLPGSGLRTIIVASDGRPSGVPDRFSMSKSGNTATLTISSLQAEDEADYFCGSSDRTQYTGVFGSGTRLTVLG

SEQ ID NO: 417 - protein, 242 aa, synthetic construct
  REGION 1..242: note = 412B9 amino acid sequence
QVQLQESGPSLVKPSQTLSLTCTVSGFSVISDSVAWVRQAPGKVPEWLGASGSSGNKYYNPALKSRLSITRDTSKSQVSLSLSSVTTEDTAVYYCARGIIAGVDVWGRGLLVSVSSEGKSSGASGESKVDDQAVLTQPSSVSGALGQRVSITCSGSSSNVGYGDYVGWYQQVPGSAPKLLIYGAASRASGVPDRFSGSRSGNTATLTISSLQAEDEADYYCASYDSSDGGVFGSGTRLTVLG

SEQ ID NO: 418 - protein, 247 aa, synthetic construct
  REGION 1..247: note = 3aA6 amino acid sequence
QVRLQESGSSLVKPSQTLSLVCTVSGFPLTSNAVGWVRQAPGKAPEWLGLIDIDGDTAYNPALESRLSITRDTSKSQVSLSLSSVAIEDTAVYYCARHYDKWGYADSIDYWGPGLLVTVSSEGKSSGASGESKVDDQALLTQPSSVFGSLGQRVSITCSGSSSNVGYGDYVGWYQQVPGSAPKLLIYDATTRASGVPDRFSGSRSGNTATLTISSLQAEDEADYYCASYQNERSGVFGSGTRLTVLG

SEQ ID NO: 419 - protein, 244 aa, synthetic construct
  REGION 1..244: note = 3aD6 amino acid sequence
QVQLQESGPSLVKPSQTLSLTCTVSGFSLTSNAVIWVRQAPGKAPEWVALIDVDGDAAYDPALKSRLSITRDTSKSQVSLSLRSVTTEDTAVYYCARDYGSWGYVSDIDYWGPGLLVTVSSEGKSSGASGESKVDDQAVLTQPSSVSGSLGQRVSITCSGSDIGGADVGWFQQVPGSGLRTLIYDNDNRPSGVPDRFSGSKSGNTATLTISSLQPEDEADYFCGTYSGANYGIFGSGTRLTVLG

SEQ ID NO: 420 - protein, 248 aa, synthetic construct
  REGION 1..248: note = 3aB7 amino acid sequence
QVQLQESGPSLVKPSQTLSLTCTVSGFSLTSNGVGWVRQAPGKVPEWVGDKSSAGKTYGNPALKSRLSITRDTSKSQVSLSLSSVTTEDTAVYYCVRCRDGGVTYGYDVDYWGPGLLVTVSSEGKSSGASGESKVDDQAVLTQPSSVSKSLGQSVSITCSGSSSNVGYGDVVSWFQQFPGSAPKLLIFGTTTRASGVPDRFSGSRSGNAATLTINSLQAEDEADYYCASFDSDSGGIAGSGTRLTVLG

SEQ ID NO: 421 - protein, 248 aa, synthetic construct
  REGION 1..248: note = 3bF4 amino acid sequence
QVQLQESGPSLVKPSQTLSLTCTVSGFSLSSNGVGWVRQAPGKVPEWLGDISSVGKKYANPALKSRLSFTRDTSKSQVSLSLSSVTTEDTAVYYCVKCRDGGVTYGYDIDYWGPGLLVTVSSEGKSSGASGESKVDDQAVLTQPSSVSKSTGQTVSITCSGSSSNVGLRNYVTWFQQVPGSAPKLLIYGATSRASGIPDRFSGSRSGNTATLIISSLQAEDEADYYCASADTNDGGVFGSGTRLTVLG

SEQ ID NO: 422 - protein, 248 aa, synthetic construct
  REGION 1..248: note = 3aD3 amino acid sequence
QVQLQESGPSLVKPSQTLSLTCTVSGFSLTSNGVGWVRRAPGKVPEWVGDISSGGKVYGHPALKSRLSITRDTSKSQVSLSVSSVTSEDTAVYYCVRCRDGGVSYGYDSDYWGPGLLVTVSSEGKSSGASGESKVDDQAVVTQPSSVSKSLGQSVSITCSGSSSNVGYGDYVGWFQQVPGSAPKLLIYGVTERASGVPDRFSGSRSGNTATLTISSIQAEDEADYYCASYDDSSGGIFGSGTRLTVLG

SEQ ID NO: 423 - protein, 248 aa, synthetic construct
  REGION 1..248: note = 3aH6 amino acid sequence
QVQLQESGPSLVKPSQTLSLTCTVSGFSLSSNGVGWVRQAPGKVPEWLGDISSVGKKYANPALKSRLSFTRDTSKSQVSLSLSSVTTEDTAVYYCVKCRDGGVTYGYDIDYWGPGLLVTASSEGKSSGASGESKVDDQAVVTQPSSVSGSLGQSVSITCSGSSGNVGYGDYVSWFQQFHGSAPKLLIYGATNLASGVPARFSGSRSGNTATLTISSLHAEDEADYYCASYDSSSGGVFGSGTRLTVLG

SEQ ID NO: 424 - protein, 248 aa, synthetic construct
  REGION 1..248: note = 3aG3 amino acid sequence
QVQLQESGPSLVKPSQTLSLTCTVSGFSLSSNGVGWVRQAPGKVPEWVGDISSSGKAYANPALKSRLSITRDTAKTQVFLSLSSVTTEDTAVYYCVRCRDGGVSYGYDIDYWGPGLLVTVSSEGKSSGASGESKVDDQAVLTQPPSVSGSPGQRVSITCSGSSSNIGGGNYLSWFQQVPGSAPKLLIYGATSRASGVPDRFSGSRSGNTATLTISSLQAEDEADYYCASFDTSSGGIFGAGTRLTVLG

SEQ ID NO: 425 - protein, 248 aa, synthetic construct
  REGION 1..248: note = 3bG4 amino acid sequence
QVQLQESGPSLVKPSQTLSLTCTISGFSLISNGVGWVRQAPGKVPEWVGDIASSGKAYSNPALKSRLSITRDTSKSQVSLSLRSVTTEDTAVYYCVRCRDGGVTYGYDIDYWGPGLLVTVSSEGKSSGASGESKVDDQAVLTQPSSVSKSLGQSVSITCSGSTSNVGSGNDVSWFQQVPGSAPKLLFYGATNRASGVPDRFSGSRSGNTATLTITSLQAEDEADYYCGSYDSNSGGIFGSGTRLTVLG

SEQ ID NO: 426 - skipped

SEQ ID NO: 427 - protein, 16 aa, synthetic construct
  REGION 1..16: note = 297-390 VHCDR2 alternative consensus sequence
  VARIANT 13: note = A or V
  VARIANT 4: note = T or S
  VARIANT 10: note = Y or F
  VARIANT 15: note = N or K
GIDXDGEEGXNPXLXS

SEQ ID NO: 428 - protein, 15 aa, synthetic construct
  REGION 1..15: note = 297-390 VHCDR3 alternative consensus sequence
  VARIANT 4: note = A, T or S
  VARIANT 1: note = S or T
  VARIANT 7: note = L or Y
XYRXDGXGYVQAIDY

SEQ ID NO: 429 - protein, 11 aa, synthetic construct
  REGION 1..11: note = 297-390 VLCDR1 alternative consensus sequence
  VARIANT 4: note = F or Y
  VARIANT 7: note = I or S
  VARIANT 9: note = S or G
SGSXIGXSXVG

SEQ ID NO: 430 - protein, 6 aa, synthetic construct
  REGION 1..6: note = 430 hexapeptide
VQIVYK

SEQ ID NO: 431 - DNA, 747 bp, synthetic construct
  misc_feature 1..747: note = NS2A1 nucleotide sequence
  source 1..747: mol_type = other DNA
caggtgcagttgcaggagtcgggacccagcctcgtgaagccctcacagaccctctccctcacctgcacggtctctggattctcattaagcactaattctgtgggctgggtccgacaggctccaggaaaggcgccggagtgggttgctggtatagatactgatggagaagaaggctttaatccagtccttaagtcccggctcagcatcaccagggacacctccaagagtcaagtctctttgtcattgagcaacgtgacaagtgaagacacggccgtgtactactgtggaagaagttataggactgatggtcttgcttacggttatgtccaagccatcgactactggggcccaggactcctggtcactatctcctcagaaggtaaatcttctggcgcgtctggcgagtctaaagtggatgaccagtctgtgctgactcagccgtcctccgtgtccgggtccccgggccagacagtctccatcacctgctctggaagctatatcggtagtagtggtgtaggctggttccaacagctcccaggatcgggcctcagaaccatcatcgtggcgagtgacggtcgaccctcaggggtccccgacggattctctatgtccaaatcgggcaacacagccaccctgaccatcagctcgctccaggctgaggacgaggccgattatttctgtggaagtagtgataggactcaatatactggacttttcggcagcgggaccaggctgaccgtcctgggt

SEQ ID NO: 432 - protein, 11 aa, synthetic construct
  REGION 1..11: note = 297-390 VLCDR3 alternative consensus sequence
  VARIANT 11: note = V or L
GSSDRTQYTGX

SEQ ID NO: 433 - protein, 112 aa, synthetic construct
  REGION 1..112: note = S1D12 antibody variable light chain
QAVLTQPSSVSGSLGQRVSITCSGSSSNVGGGNSVGWYQHLPGSGLKTIIYDTNSRPSGVPDRFSGSRSGNTATLTINSLQAEDEGDYYCVTGDSTTHDDLVGSGTRLTVLG

SEQ ID NO: 434 - protein, 238 aa, synthetic construct
  REGION 1..238: note = 412G11 amino acid sequence
QVRLQESGPSLVKPSQTLSLTCTVSGFSLTSYALGWVRQAPGRAPEWIGNIWRGGRIEYNPALKSRLSITRDTSKSQVSLSLSSVTTEDTAVYYCSRSGGDWGPGLLVTVSSEGKSSGASGESKVDDQAVLTQPSSVSGSLGQRVSITCSGSSSNVGYGNYVGWYQQVPGSAPKLLIYGATSRASGVPDRFSGSRSENTATLTISSLQAEDEADYYCASYDRSESVVFGSGTRLTVLG

SEQ ID NO: 435 - protein, 30 aa, synthetic construct
  REGION 1..30: note = S1D12 VHFR1
QVQLQESGPSLVKPSQTLSLTCTVSGFSLN

SEQ ID NO: 436 - protein, 14 aa, synthetic construct
  REGION 1..14: note = S1D12 VHFR2
WVRQAPGKVPESLV

SEQ ID NO: 437 - protein, 32 aa, synthetic construct
  REGION 1..32: note = S1D12 VHFR3
RLDITRDTSKNQISLSLSSVTTDDAAVYYCTR

SEQ ID NO: 438 - protein, 11 aa, synthetic construct
  REGION 1..11: note = S1D12 VHFR4
WGPGLLVTVSS

SEQ ID NO: 439 - protein, 22 aa, synthetic construct
  REGION 1..22: note = S1D12 VLFR1
QAVLTQPSSVSGSLGQRVSITC

SEQ ID NO: 440 - protein, 15 aa, synthetic construct
  REGION 1..15: note = S1D12 VLFR2
WYQHLPGSGLKTIIY

SEQ ID NO: 441 - protein, 32 aa, synthetic construct
  REGION 1..32: note = S1D12 VLFR3
GVPDRFSGSRSGNTATLTINSLQAEDEGDYYC

SEQ ID NO: 442 - protein, 11 aa, synthetic construct
  REGION 1..11: note = S1D12 VLFR4
VGSGTRLTVLG

SEQ ID NO: 443 - protein, 124 aa, synthetic construct; identical to SEQ ID NO: 62
  REGION 1..124: note = S1D12 VH Domain

SEQ ID NO: 444 - protein, 112 aa, synthetic construct; identical to SEQ ID NO: 433
  REGION 1..112: note = S1D12 VL Domain

SEQ ID NO: 445 - protein, 454 aa, synthetic construct
  REGION 1..454: note = S1D12 mAB Heavy Chain
QVQLQESGPSLVKPSQTLSLTCTVSGFSLNNNAVGWVRQAPGKVPESLVGCSSDGTCYYNSALKSRLDITRDTSKNQISLSLSSVTTDDAAVYYCTRGHYSIYGYDYLGTIDYWGPGLLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 446 - protein, 218 aa, synthetic construct
  REGION 1..218: note = S1D12 mAB Light Chain
QAVLTQPSSVSGSLGQRVSITCSGSSSNVGGGNSVGWYQHLPGSGLKTIIYDTNSRPSGVPDRFSGSRSGNTATLTINSLQAEDEGDYYCVTGDSTTHDDLVGSGTRLTVLGGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 447 - protein, 30 aa, synthetic construct
  REGION 1..30: note = S1G2 VHFR1
QVQLQESGPSLVKPSQTLSLTCTVSGFSLT

SEQ ID NO: 448 - protein, 14 aa, synthetic construct
  REGION 1..14: note = S1G2 VHFR2
WVRQAPGKAPEWVA

SEQ ID NO: 449 - protein, 32 aa, synthetic construct
  REGION 1..32: note = S1G2 VHFR3
RLSITRDTSKSQVSLSLSSVTSEDTAVYYCGR

SEQ ID NO: 450 - protein, 11 aa, synthetic construct
  REGION 1..11: note = S1G2 VHFR4
WGPGLLVTVSS

SEQ ID NO: 451 - protein, 22 aa, synthetic construct
  REGION 1..22: note = S1G2 VLFR1
QAVVTQPSSVSGSLGQRVSITC

SEQ ID NO: 452 - protein, 15 aa, synthetic construct
  REGION 1..15: note = S1G2 VLFR2
WFQQLPGSGLRTIIV

SEQ ID NO: 453 - protein, 32 aa, synthetic construct
  REGION 1..32: note = S1G2 VLFR3
GVPDRFSMSKSGNTATLTISSLQAEDEADYFC

SEQ ID NO: 454 - protein, 11 aa, synthetic construct
  REGION 1..11: note = S1G2 VLFR4
FGSGTRLTVLG

SEQ ID NO: 455 - protein, 125 aa, synthetic construct
  REGION 1..125: note = S1G2 VH Domain
QVQLQESGPSLVKPSQTLSLTCTVSGFSLTSNSVGWVRQAPGKAPEWVAGIDTDGEEGYNPALNSRLSITRDTSKSQVSLSLSSVTSEDTAVYYCGRSYRADGLAYGYVQAIDYWGPGLLVTVSS

SEQ ID NO: 456 - protein, 109 aa, synthetic construct
  REGION 1..109: note = S1G2 VL Domain
QAVVTQPSSVSGSLGQRVSITCSGSFIGISSVGWFQQLPGSGLRTIIVASDGRPSGVPDRFSMSKSGNTATLTISSLQAEDEADYFCGSSDRTPYTGVFGSGTRLTVLG

SEQ ID NO: 457 - protein, 455 aa, synthetic construct
  REGION 1..455: note = S1G2 mAB Heavy Chain
QVQLQESGPSLVKPSQTLSLTCTVSGFSLTSNSVGWVRQAPGKAPEWVAGIDTDGEEGYNPALNSRLSITRDTSKSQVSLSLSSVTSEDTAVYYCGRSYRADGLAYGYVQAIDYWGPGLLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 458 - protein, 215 aa, synthetic construct
  REGION 1..215: note = S1G2 mAB Light Chain
QAVVTQPSSVSGSLGQRVSITCSGSFIGISSVGWFQQLPGSGLRTIIVASDGRPSGVPDRFSMSKSGNTATLTISSLQAEDEADYFCGSSDRTPYTGVFGSGTRLTVLGGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 459 - protein, 30 aa, synthetic construct
  REGION 1..30: note = 412E10 VHFR1
QVQLQESGPSLVKPSQTLSLTCTVSGFSVI

SEQ ID NO: 460 - protein, 14 aa, synthetic construct
  REGION 1..14: note = 412E10 VHFR2
WVRQAPGKVPEWLG

SEQ ID NO: 461 - protein, 32 aa, synthetic construct
  REGION 1..32: note = 412E10 VHFR3
RLSITRDTSKSQVSLSLSSVTTEDTAVYYCAR

SEQ ID NO: 462 - protein, 11 aa, synthetic construct
  REGION 1..11: note = 412E10 VHFR4
WGRGLLVTVSS

SEQ ID NO: 463 - protein, 22 aa, synthetic construct
  REGION 1..22: note = 412E10 VLFR1
QAVLTQPSSVSGSLGQRVSITC

SEQ ID NO: 464 - protein, 15 aa, synthetic construct
  REGION 1..15: note = 412E10 VLFR2
WYQQVPGSAPKLLIY

SEQ ID NO: 465 - protein, 32 aa, synthetic construct
  REGION 1..32: note = 412E10 VLFR3
GVPDRFSGSRSGDTATLTITSLQAEDEADYYC

SEQ ID NO: 466 - protein, 11 aa, synthetic construct
  REGION 1..11: note = 412E10 VLFR4
FGSGTRLTVLG

SEQ ID NO: 467 - protein, 116 aa, synthetic construct
  REGION 1..116: note = 412E10 VH Domain
QVQLQESGPSLVKPSQTLSLTCTVSGFSVISDSVAWVRQAPGKVPEWLGASGSSGNKYYNPALKSRLSITRDTSKSQVSLSLSSVTTEDTAVYYCARGIIAGVDVWGRGLLVTVSS

SEQ ID NO: 468 - protein, 110 aa, synthetic construct
  REGION 1..110: note = 412E10 VL Domain
QAVLTQPSSVSGSLGQRVSITCSGSSSNVGYGNYVGWYQQVPGSAPKLLIYGTAIRASGVPDRFSGSRSGDTATLTITSLQAEDEADYYCASYQSNYAFFGSGTRLTVLG

SEQ ID NO: 469 - protein, 446 aa, synthetic construct
  REGION 1..446: note = 412E10 mAB Heavy Chain
QVQLQESGPSLVKPSQTLSLTCTVSGFSVISDSVAWVRQAPGKVPEWLGASGSSGNKYYNPALKSRLSITRDTSKSQVSLSLSSVTTEDTAVYYCARGIIAGVDVWGRGLLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 470 - protein, 216 aa, synthetic construct
  REGION 1..216: note = 412E10 mAB Light Chain
QAVLTQPSSVSGSLGQRVSITCSGSSSNVGYGNYVGWYQQVPGSAPKLLIYGTAIRASGVPDRFSGSRSGDTATLTITSLQAEDEADYYCASYQSNYAFFGSGTRLTVLGGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 471 - protein, 30 aa, synthetic construct
  REGION 1..30: note = 3bG4 VHFR1
QVQLQESGPSLVKPSQTLSLTCTISGFSLI

SEQ ID NO: 472 - protein, 14 aa, synthetic construct
  REGION 1..14: note = 3bG4 VHFR2
WVRQAPGKVPEWVG

SEQ ID NO: 473 - protein, 32 aa, synthetic construct
  REGION 1..32: note = 3bG4 VHFR3
RLSITRDTSKSQVSLSLRSVTTEDTAVYYCVR

SEQ ID NO: 474 - protein, 11 aa, synthetic construct
  REGION 1..11: note = 3bG4 VHFR4
WGPGLLVTVSS

SEQ ID NO: 475 - protein, 22 aa, synthetic construct
  REGION 1..22: note = 3bG4 VLFR1
QAVLTQPSSVSKSLGQSVSITC

SEQ ID NO: 476 - protein, 15 aa, synthetic construct
  REGION 1..15: note = 3bG4 VLFR2
WFQQVPGSAPKLLFY

SEQ ID NO: 477 - protein, 32 aa, synthetic construct
  REGION 1..32: note = 3bG4 VLFR3
GVPDRFSGSRSGNTATLTITSLQAEDEADYYC

SEQ ID NO: 478 - protein, 11 aa, synthetic construct
  REGION 1..11: note = 3bG4 VLFR4
FGSGTRLTVLG

SEQ ID NO: 479 - protein, 122 aa, synthetic construct
  REGION 1..122: note = 3bG4 VH Domain
QVQLQESGPSLVKPSQTLSLTCTISGFSLISNGVGWVRQAPGKVPEWVGDIASSGKAYSNPALKSRLSITRDTSKSQVSLSLRSVTTEDTAVYYCVRCRDGGVTYGYDIDYWGPGLLVTVSS

SEQ ID NO: 480 - protein, 111 aa, synthetic construct
  REGION 1..111: note = 3bG4 VL Domain
QAVLTQPSSVSKSLGQSVSITCSGSTSNVGSGNDVSWFQQVPGSAPKLLFYGATNRASGVPDRFSGSRSGNTATLTITSLQAEDEADYYCGSYDSNSGGIFGSGTRLTVLG

SEQ ID NO: 481 - protein, 452 aa, synthetic construct
  REGION 1..452: note = 3bG4 mAB Heavy Chain
QVQLQESGPSLVKPSQTLSLTCTISGFSLISNGVGWVRQAPGKVPEWVGDIASSGKAYSNPALKSRLSITRDTSKSQVSLSLRSVTTEDTAVYYCVRCRDGGVTYGYDIDYWGPGLLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 482 - protein, 217 aa, synthetic construct
  REGION 1..217: note = 3bG4 mAB Light Chain
QAVLTQPSSVSKSLGQSVSITCSGSTSNVGSGNDVSWFQQVPGSAPKLLFYGATNRASGVPDRFSGSRSGNTATLTITSLQAEDEADYYCGSYDSNSGGIFGSGTRLTVLGGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 483 - protein, 30 aa, synthetic construct; identical to SEQ ID NO: 447
  REGION 1..30: note = E2E8 VHFR1

SEQ ID NO: 484 - protein, 14 aa, synthetic construct
  REGION 1..14: note = E2E8 VHFR2
WVRQAPGKALEWVG

SEQ ID NO: 485 - protein, 32 aa, synthetic construct
  REGION 1..32: note = E2E8 VHFR3
RLSITRDTSKSQVFLSLSSVTTEDMAMYYCAR

SEQ ID NO: 486 - protein, 11 aa, synthetic construct
  REGION 1..11: note = E2E8 VHFR4
WGRGLLVTVSS

SEQ ID NO: 487 - protein, 22 aa, synthetic construct
  REGION 1..22: note = E2E8 VLFR1
QAVLTQPSSVSKSLGQSVSIAC

SEQ ID NO: 488 - protein, 15 aa, synthetic construct
  REGION 1..15: note = E2E8 VLFR2
WFQQIPGSAPKLLIY

SEQ ID NO: 489 - protein, 32 aa, synthetic construct
  REGION 1..32: note = E2E8 VLFR3
GVPDRFSGARSGNTATLTINSLQAEDEADYYC

SEQ ID NO: 490 - protein, 11 aa, synthetic construct
  REGION 1..11: note = E2E8 VLFR4
FGSGTRLTVLG

SEQ ID NO: 491 - protein, 117 aa, synthetic construct
  REGION 1..117: note = E2E8 VH Domain
QVQLQESGPSLVKPSQTLSLTCTVSGFSLTDRGVAWVRQAPGKALEWVGTMRSGGTIDYNPALKSRLSITRDTSKSQVFLSLSSVTTEDMAMYYCARGYLSGDRYAWGRGLLVTVSS

SEQ ID NO: 492 - protein, 111 aa, synthetic construct
  REGION 1..111: note = E2E8 VL Domain
QAVLTQPSSVSKSLGQSVSIACSGSRSDIGYGNYVSWFQQIPGSAPKLLIYDTNTRASGVPDRFSGARSGNTATLTINSLQAEDEADYYCANIDSSRSHLFGSGTRLTVLG

SEQ ID NO: 493 - protein, 447 aa, synthetic construct
  REGION 1..447: note = E2E8 mAB Heavy Chain
QVQLQESGPSLVKPSQTLSLTCTVSGFSLTDRGVAWVRQAPGKALEWVGTMRSGGTIDYNPALKSRLSITRDTSKSQVFLSLSSVTTEDMAMYYCARGYLSGDRYAWGRGLLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 494 - protein, 217 aa, synthetic construct
  REGION 1..217: note = E2E8 mAB Light Chain
QAVLTQPSSVSKSLGQSVSIACSGSRSDIGYGNYVSWFQQIPGSAPKLLIYDTNTRASGVPDRFSGARSGNTATLTINSLQAEDEADYYCANIDSSRSHLFGSGTRLTVLGGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 495 - protein, 30 aa, synthetic construct
  REGION 1..30: note = CA7 VHFR1
RVRLQGSGPSLVKPSQTLSLTCTVSGFSFD

SEQ ID NO: 496 - protein, 14 aa, synthetic construct
  REGION 1..14: note = CA7 VHFR2
WVRQAPGKALEWLG

SEQ ID NO: 497 - protein, 32 aa, synthetic construct
  REGION 1..32: note = CA7 VHFR3
RLSITRDTSKSQVSLSVSSVTIEDTALYYCVR

SEQ ID NO: 498 - protein, 11 aa, synthetic construct
  REGION 1..11: note = CA7 VHFR4
WSPGLLVTVSS

SEQ ID NO: 499 - protein, 22 aa, synthetic construct
  REGION 1..22: note = CA7 VLFR1
QVVRTQPSSVSGSLGQRVSITC

SEQ ID NO: 500 - protein, 15 aa, synthetic construct
  REGION 1..15: note = CA7 VLFR2
WFQQVPGSAPKLLIY

SEQ ID NO: 501 - protein, 32 aa, synthetic construct
  REGION 1..32: note = CA7 VLFR3
GVPDRFSGSRSGNTATLTIDSLQAEDEADYYC

SEQ ID NO: 502 - protein, 11 aa, synthetic construct
  REGION 1..11: note = CA7 VLFR4
FGSGTRLTVLG

SEQ ID NO: 503 - protein, 125 aa, synthetic construct
  REGION 1..125: note = CA7 VH Domain
RVRLQGSGPSLVKPSQTLSLTCTVSGFSFDSYYVGWVRQAPGKALEWLGNIYSTGRAFYNPALKSRLSITRDTSKSQVSLSVSSVTIEDTALYYCVRGSYYHGGGNGMVDFFDYWSPGLLVTVSS

SEQ ID NO: 504 - protein, 111 aa, synthetic construct
  REGION 1..111: note = CA7 VL Domain
QVVRTQPSSVSGSLGQRVSITCSGSSSNVGYGNYVGWFQQVPGSAPKLLIYAATSRASGVPDRFSGSRSGNTATLTIDSLQAEDEADYYCSSYQRGNTGVFGSGTRLTVLG

SEQ ID NO: 505 - protein, 455 aa, synthetic construct
  REGION 1..455: note = CA7 mAB Heavy Chain
RVRLQGSGPSLVKPSQTLSLTCTVSGFSFDSYYVGWVRQAPGKALEWLGNIYSTGRAFYNPALKSRLSITRDTSKSQVSLSVSSVTIEDTALYYCVRGSYYHGGGNGMVDFFDYWSPGLLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 506 - protein, 217 aa, synthetic construct
  REGION 1..217: note = CA7 mAB Light Chain
QVVRTQPSSVSGSLGQRVSITCSGSSSNVGYGNYVGWFQQVPGSAPKLLIYAATSRASGVPDRFSGSRSGNTATLTIDSLQAEDEADYYCSSYQRGNTGVFGSGTRLTVLGGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 507 - protein, 30 aa, synthetic construct; identical to SEQ ID NO: 447
  REGION 1..30: note = CB7 VHFR1

SEQ ID NO: 508 - protein, 14 aa, synthetic construct
  REGION 1..14: note = CB7 VHFR2
WVRQAPGKALEWVS

SEQ ID NO: 509 - protein, 32 aa, synthetic construct
  REGION 1..32: note = CB7 VHFR3
RLSITADTSKSQVSLSLSSVTTEDTAVYYCAR

SEQ ID NO: 510 - protein, 11 aa, synthetic construct
  REGION 1..11: note = CB7 VHFR4
WGPGLLVTVSS

SEQ ID NO: 511 - protein, 22 aa, synthetic construct
  REGION 1..22: note = CB7 VLFR1
QAVLTQPSSVSRSLGQSVSMTC

SEQ ID NO: 512 - protein, 15 aa, synthetic construct
  REGION 1..15: note = CB7 VLFR2
WFQQVPGSAPKLLIY

SEQ ID NO: 513 - protein, 32 aa, synthetic construct
  REGION 1..32: note = CB7 VLFR3
GVPDRFSGSRSGNTATLTISSLQAEDEADYYC

SEQ ID NO: 514 - protein, 11 aa, synthetic construct
  REGION 1..11: note = CB7 VLFR4
FGSGTRLTVLG

SEQ ID NO: 515 - protein, 119 aa, synthetic construct
  REGION 1..119: note = CB7 VH Domain
QVQLQESGPSLVKPSQTLSLTCTVSGFSLTNYRVGWVRQAPGKALEWVSNIRSGGTTWYNPALKSRLSITADTSKSQVSLSLSSVTTEDTAVYYCARDSSGDLYAYDYWGPGLLVTVSS

SEQ ID NO: 516 - protein, 111 aa, synthetic construct
  REGION 1..111: note = CB7 VL Domain
QAVLTQPSSVSRSLGQSVSMTCSGSSSNVGYGNYMAWFQQVPGSAPKLLIYGATSRASGVPDRFSGSRSGNTATLTISSLQAEDEADYYCASYDSTSGGVFGSGTRLTVLG

SEQ ID NO: 517 - protein, 449 aa, synthetic construct
  REGION 1..449: note = CB7 mAB Heavy Chain
QVQLQESGPSLVKPSQTLSLTCTVSGFSLTNYRVGWVRQAPGKALEWVSNIRSGGTTWYNPALKSRLSITADTSKSQVSLSLSSVTTEDTAVYYCARDSSGDLYAYDYWGPGLLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 518 - protein, 217 aa, synthetic construct
  REGION 1..217: note = CB7 mAB Light Chain
QAVLTQPSSVSRSLGQSVSMTCSGSSSNVGYGNYMAWFQQVPGSAPKLLIYGATSRASGVPDRFSGSRSGNTATLTISSLQAEDEADYYCASYDSTSGGVFGSGTRLTVLGGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 519 - protein, 30 aa, synthetic construct; identical to SEQ ID NO: 447
  REGION 1..30: note = CC7 VHFR1

SEQ ID NO: 520 - protein, 14 aa, synthetic construct
  REGION 1..14: note = CC7 VHFR2
WVRQAPGKAPEWVA

SEQ ID NO: 521 - protein, 32 aa, synthetic construct
  REGION 1..32: note = CC7 VHFR3
RLSITRDTSKSQVSLSLRSVTTEDTAVYYCAR

SEQ ID NO: 522 - protein, 11 aa, synthetic construct
  REGION 1..11: note = CC7 VHFR4
WGPGLLVTVSS

SEQ ID NO: 523 - protein, 22 aa, synthetic construct
  REGION 1..22: note = CC7 VLFR1
RVVRTQPSSVSGSLGQRVSITC

SEQ ID NO: 524 - protein, 15 aa, synthetic construct
  REGION 1..15: note = CC7 VLFR2
WFQQVPGSGLKTVIY

SEQ ID NO: 525 - protein, 32 aa, synthetic construct
  REGION 1..32: note = CC7 VLFR3
GVPDRFSGSKSGDTATLTISSLQAEDEADYYC

SEQ ID NO: 526 - protein, 11 aa, synthetic construct
  REGION 1..11: note = CC7 VLFR4
FGSGTRLTVLG

SEQ ID NO: 527 - protein, 121 aa, synthetic construct
  REGION 1..121: note = CC7 VH Domain
QVQLQESGPSLVKPSQTLSLTCTVSGFSLTSNAVIWVRQAPGKAPEWVALIDVDGDAAYDPALKSRLSITRDTSKSQVSLSLRSVTTEDTAVYYCARDYGSWGYVSDIDYWGPGLLVTVSS

SEQ ID NO: 528 - protein, 108 aa, synthetic construct
  REGION 1..108: note = CC7 VL Domain
RVVRTQPSSVSGSLGQRVSITCSGSYITGSSVGWFQQVPGSGLKTVIYDNNDRPSGVPDRFSGSKSGDTATLTISSLQAEDEADYYCASYDTSNIGLFGSGTRLTVLG

SEQ ID NO: 529 - protein, 451 aa, synthetic construct
  REGION 1..451: note = CC7 mAB Heavy Chain
QVQLQESGPSLVKPSQTLSLTCTVSGFSLTSNAVIWVRQAPGKAPEWVALIDVDGDAAYDPALKSRLSITRDTSKSQVSLSLRSVTTEDTAVYYCARDYGSWGYVSDIDYWGPGLLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 530 - protein, 214 aa, synthetic construct
  REGION 1..214: note = CC7 mAB Light Chain
RVVRTQPSSVSGSLGQRVSITCSGSYITGSSVGWFQQVPGSGLKTVIYDNNDRPSGVPDRFSGSKSGDTATLTISSLQAEDEADYYCASYDTSNIGLFGSGTRLTVLGGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 531 - protein, 30 aa, synthetic construct; identical to SEQ ID NO: 447
  REGION 1..30: note = CE2 VHFR1

SEQ ID NO: 532 - protein, 14 aa, synthetic construct
  REGION 1..14: note = CE2 VHFR2
WVRQAPGKALEWIG

SEQ ID NO: 533 - protein, 32 aa, synthetic construct
  REGION 1..32: note = CE2 VHFR3
RLSITRDTSKNQVSLSLSSATTEDTAVYYCGR

SEQ ID NO: 534 - protein, 11 aa, synthetic construct
  REGION 1..11: note = CE2 VHFR4
WGPGLLVTVSS

SEQ ID NO: 535 - protein, 22 aa, synthetic construct
  REGION 1..22: note = CE2 VLFR1
QAVLTQPSSVSKSLGQSVSITC

SEQ ID NO: 536 - protein, 15 aa, synthetic construct
  REGION 1..15: note = CE2 VLFR2
WFQQVPGSAPKILIY

SEQ ID NO: 537 - protein, 32 aa, synthetic construct; identical to SEQ ID NO: 477
  REGION 1..32: note = CE2 VLFR3

SEQ ID NO: 538 - protein, 11 aa, synthetic construct
  REGION 1..11: note = CE2 VLFR4
FGSGTRLTVLG

SEQ ID NO: 539 - protein, 124 aa, synthetic construct
  REGION 1..124: note = CE2 VH Domain
QVQLQESGPSLVKPSQTLSLTCTVSGFSLTNYPVGWVRQAPGKALEWIGNIENDGSANYASALKSRLSITRDTSKNQVSLSLSSATTEDTAVYYCGREFGGSDGYTYFVDIDYWGPGLLVTVSS

SEQ ID NO: 540 - protein, 111 aa, synthetic construct
  REGION 1..111: note = CE2 VL Domain
QAVLTQPSSVSKSLGQSVSITCSGSSSNVGYGNYVSWFQQVPGSAPKILIYGATSRASGVPDRFSGSRSGNTATLTITSLQAEDEADYYCASYDGSSSGVFGSGTRLTVLG

SEQ ID NO: 541 - protein, 454 aa, synthetic construct
  REGION 1..454: note = CE2 mAB Heavy Chain
QVQLQESGPSLVKPSQTLSLTCTVSGFSLTNYPVGWVRQAPGKALEWIGNIENDGSANYASALKSRLSITRDTSKNQVSLSLSSATTEDTAVYYCGREFGGSDGYTYFVDIDYWGPGLLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 542 - protein, 217 aa, synthetic construct
  REGION 1..217: note = CE2 mAB Light Chain
QAVLTQPSSVSKSLGQSVSITCSGSSSNVGYGNYVSWFQQVPGSAPKILIYGATSRASGVPDRFSGSRSGNTATLTITSLQAEDEADYYCASYDGSSSGVFGSGTRLTVLGGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 543 - protein, 30 aa, synthetic construct
  REGION 1..30: note = CE3 VHFR1
QVRLQESGPSLVKPSQTLSVTCTVSGFSLI

SEQ ID NO: 544 - protein, 14 aa, synthetic construct
  REGION 1..14: note = CE3 VHFR2
WVRQAPGKVPESLA

SEQ ID NO: 545 - protein, 32 aa, synthetic construct; identical to SEQ ID NO: 437
  REGION 1..32: note = CE3 VHFR3

SEQ ID NO: 546 - protein, 11 aa, synthetic construct
  REGION 1..11: note = CE3 VHFR4
WGPGLLVTVSS

SEQ ID NO: 547 - protein, 22 aa, synthetic construct
  REGION 1..22: note = CE3 VLFR1
QAVLTQPSSVSGSLGQRVSITC

SEQ ID NO: 548 - protein, 15 aa, synthetic construct
  REGION 1..15: note = CE3 VLFR2
WFQQLPGSGLRTIIY

SEQ ID NO: 549 - protein, 32 aa, synthetic construct
  REGION 1..32: note = CE3 VLFR3
GIPDRFSGSKSGVTATLTIDSLQAEDEADYFC

SEQ ID NO: 550 - protein, 11 aa, synthetic construct
  REGION 1..11: note = CE3 VLFR4
FGSGTRLTVLG

SEQ ID NO: 551 - protein, 124 aa, synthetic construct
  REGION 1..124: note = CE3 VH Domain
QVRLQESGPSLVKPSQTLSVTCTVSGFSLISNAVGWVRQAPGKVPESLAGCSSDGKCYYNSALKSRLDITRDTSKNQISLSLSSVTTDDAAVYYCTRGYYPVYGYDYLGTIDYWGPGLLVTVSS

SEQ ID NO: 552 - protein, 111 aa, synthetic construct
  REGION 1..111: note = CE3 VL Domain
QAVLTQPSSVSGSLGQRVSITCSGSSSNVGRNDVAWFQQLPGSGLRTIIYGTTSRPSGIPDRFSGSKSGVTATLTIDSLQAEDEADYFCASGDSSAINDIFGSGTRLTVLG

SEQ ID NO: 553 - protein, 454 aa, synthetic construct
  REGION 1..454: note = CE3 mAB Heavy Chain
QVRLQESGPSLVKPSQTLSVTCTVSGFSLISNAVGWVRQAPGKVPESLAGCSSDGKCYYNSALKSRLDITRDTSKNQISLSLSSVTTDDAAVYYCTRGYYPVYGYDYLGTIDYWGPGLLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 554 - protein, 217 aa, synthetic construct
  REGION 1..217: note = CE3 mAB Light Chain
QAVLTQPSSVSGSLGQRVSITCSGSSSNVGRNDVAWFQQLPGSGLRTIIYGTTSRPSGIPDRFSGSKSGVTATLTIDSLQAEDEADYFCASGDSSAINDIFGSGTRLTVLGGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 555 - protein, 30 aa, synthetic construct; identical to SEQ ID NO: 447
  REGION 1..30: note = CA4 VHFR1

SEQ ID NO: 556 - protein, 14 aa, synthetic construct
  REGION 1..14: note = CA4 VHFR2
WVRQAPGQALEWIS

SEQ ID NO: 557 - protein, 32 aa, synthetic construct
  REGION 1..32: note = CA4 VHFR3
RLSITRDTSKSQFSLSLSSVTTEDTAVYYCTR

SEQ ID NO: 558 - protein, 11 aa, synthetic construct
  REGION 1..11: note = CA4 VHFR4
WGPGLLVTVSS

SEQ ID NO: 559 - protein, 23 aa, synthetic construct
  REGION 1..23: note = CA4 VLFR1
DIQVTQSPSSLSASLTERVSITC

SEQ ID NO: 560 - protein, 15 aa, synthetic construct
  REGION 1..15: note = CA4 VLFR2
WYQQKPGQAPKLLIY

SEQ ID NO: 561 - protein, 32 aa, synthetic construct
  REGION 1..32: note = CA4 VLFR3
DVPSRFSGSGSGTDYTLTITSLEADDTATYYC

SEQ ID NO: 562 - protein, 10 aa, synthetic construct
  REGION 1..10: note = CA4 VLFR4
FGGGTNVEIK

SEQ ID NO: 563 - protein, 112 aa, synthetic construct
  REGION 1..112: note = CA4 VH Domain
QVQLQESGPSLVKPSQTLSLTCTVSGFSLTSYSVYWVRQAPGQALEWISIMYASGRVDYNPALKSRLSITRDTSKSQFSLSLSSVTTEDTAVYYCTRGIENWGPGLLVTVSS

SEQ ID NO: 564 - protein, 107 aa, synthetic construct
  REGION 1..107: note = CA4 VL Domain
DIQVTQSPSSLSASLTERVSITCRTSQSVNNYLSWYQQKPGQAPKLLIYYATRLYTDVPSRFSGSGSGTDYTLTITSLEADDTATYYCLQYDSTPLAFGGGTNVEIK

SEQ ID NO: 565 - protein, 442 aa, synthetic construct
  REGION 1..442: note = CA4 mAB Heavy Chain
QVQLQESGPSLVKPSQTLSLTCTVSGFSLTSYSVYWVRQAPGQALEWISIMYASGRVDYNPALKSRLSITRDTSKSQFSLSLSSVTTEDTAVYYCTRGIENWGPGLLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 566 - protein, 213 aa, synthetic construct
  REGION 1..213: note = CA4 mAB Light Chain
DIQVTQSPSSLSASLTERVSITCRTSQSVNNYLSWYQQKPGQAPKLLIYYATRLYTDVPSRFSGSGSGTDYTLTITSLEADDTATYYCLQYDSTPLAFGGGTNVEIKGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 567 - DNA, 372 bp, synthetic construct
  misc_feature 1..372: note = S1D12 VH Nucleotide Sequence
  source 1..372: mol_type = other DNA
caggtgcagctgcaggagtcgggacccagcctggtgaagccctcacagaccctctccctcacctgcacggtctctggattctcattgaacaacaatgctgtaggctgggtccgccaggctccaggaaaggtgccggagtcgcttgtgggttgtagcagtgatggaacgtgttactataattcggccctgaaatcccggctcgacatcaccagggacacctccaagaaccagatctccctgtcactgagcagcgttacaactgacgacgcggccgtgtactattgtacaagaggccattatagtatttatggttatgactatcttggcactatcgactactggggcccaggactcctggtcaccgtctcctca

SEQ ID NO: 568 - DNA, 336 bp, synthetic construct
  misc_feature 1..336: note = S1D12 VL Nucleotide Sequence
  source 1..336: mol_type = other DNA
caggctgtgctgactcagccgtcctccgtgtccgggtccctgggccagagggtctccatcacctgctctggaagcagcagcaacgtcgggggtggtaatagtgtgggctggtaccaacacctcccaggctcaggcctcaaaaccatcatctatgatactaacagtcgaccctcgggggtcccggaccgattctctggctccaggtctggcaacacggccaccctaaccatcaactcgctccaggctgaggacgagggtgattattactgtgtaacgggtgacagcactactcatgatgatcttgtcggcagcgggaccaggctgaccgtcctgggg

SEQ ID NO: 569 - DNA, 375 bp, synthetic construct
  misc_feature 1..375: note = S1G2 VH Nucleotide Sequence
  source 1..375: mol_type = other DNA
caggtgcagctgcaggagtcgggacccagcctggtgaagccctcacagaccctctccctcacctgcacggtctctggattctcattaaccagcaattctgtgggctgggtccgacaggctccaggaaaggcgccggagtgggttgctggtatagatactgatggagaagaaggctataatccagcccttaactcccggctcagcatcaccagggacacctccaagagtcaagtctctttgtcattgagcagcgtgacaagtgaggacacggccgtgtactactgtggaagaagttatagggctgatggtcttgcttacggttatgtccaagccatcgactactggggcccaggactcctggtcaccgtctcctca

SEQ ID NO: 570 - DNA, 327 bp, synthetic construct
  misc_feature 1..327: note = S1G2 VL Nucleotide Sequence
  source 1..327: mol_type = other DNA
caggccgtggtgactcagccgtcctccgtgtctgggtccctgggccagagggtctccatcacctgctctggaagcttcatcggtattagtagtgtaggctggttccaacagctcccaggatcgggcctcagaaccatcatcgtggcgagtgacggtcgaccctcaggggtccccgaccgattctctatgtccaaatcgggcaacacagccaccctgaccatcagctcgctccaggctgaggacgaggccgattatttctgtggaagtagtgataggactccttatactggggtcttcggcagcgggaccaggctgaccgtcctgggt

SEQ ID NO: 571 - DNA, 357 bp, synthetic construct
  misc_feature 1..357: note = CB7 VH Nucleotide Sequence
  source 1..357: mol_type = other DNA
caggtgcagctgcaggagtcgggacccagcctggtgaagccctcacagaccctctccctcacctgcacggtctctggattctcattaaccaactatcgtgtaggttgggtccgccaggctccaggaaaggcactggagtgggttagtaacatacggagtggtggaactacatggtataacccggccctgaaatcccggctcagcatcaccgcggacacctccaagagccaagtctccctgtcactgagcagcgtgacaactgaggacacggccgtatattattgtgcaagagattcctctggtgatctttatgcgtatgattactggggcccaggactcctggtcaccgtctcctca

SEQ ID NO: 572 - DNA, 333 bp, synthetic construct
  misc_feature 1..333: note = CB7 VL Nucleotide Sequence
  source 1..333: mol_type = other DNA
caggccgtgctgactcagccgtcctccgtgtccaggtccctgggccagagtgtctccatgacctgctctggaagcagcagcaacgttggatatggtaattatatggcctggttccaacaggttccaggatcagcccccaaactcctcatctatggtgcaaccagtcgagcctcgggggtccccgaccgattctccggctccaggtctggcaacacagcgactctgaccatcagctcgctccaggctgaggacgaggccgattactactgtgcatcttatgacagcactagcgggggtgtcttcggcagcgggaccaggctgaccgtcctgggt

SEQ ID NO: 573 - DNA, 363 bp, synthetic construct
  misc_feature 1..363: note = CC7 VH Nucleotide Sequence
  source 1..363: mol_type = other DNA
caggtgcagctgcaggagtcgggacccagcctggtgaagccctcacagaccctctccctcacctgcacggtctctggattctcactaaccagcaatgctgtgatctgggtccggcaggctccaggaaaggcgccggagtgggttgctttgatagatgttgatggagatgcagcctatgacccagcccttaagtcccgcctcagcatcaccagggacacctccaagagtcaagtctccctttcactgcgcagcgtgacaactgaggacacggccgtgtactactgtgcaagagactatggtagttggggttatgtttccgacatcgactactggggcccaggactcctggtcaccgtctcctca

SEQ ID NO: 574 - DNA, 324 bp, synthetic construct
  misc_feature 1..324: note = CC7 VL Nucleotide Sequence
  source 1..324: mol_type = other DNA
agggtcgtgcggactcaaccgtcctccgtgtctgggtccctgggccagagggtctccatcacctgctctggaagctacatcactggtagttctgtaggctggttccaacaggtcccaggatcgggcctcaaaaccgtcatctatgacaataacgatcgaccctcaggggtccccgaccgattctctggctccaagtcgggcgacacagccaccctgaccatcagctcgctccaggctgaggacgaggccgattattactgtgcatcttatgacaccagtaacattggtcttttcggcagcgggaccaggctgaccgtcctgggt

SEQ ID NO: 575 - DNA, 336 bp, synthetic construct
  misc_feature 1..336: note = CA4 VH Nucleotide Sequence
  source 1..336: mol_type = other DNA
caggttcagctgcaggagtcgggacccagcctggtgaagccctcacagaccctctccctcacctgcacggtctctggattctcattaaccagctattccgtatactgggtccgccaggctccaggccaggcactggagtggattagtattatgtatgctagtggaagagtagactataacccggccctgaaatcccggctcagcatcaccagggacacctccaagagtcaattctccctgtcattgagcagcgtgacaactgaggacacggccgtctactactgtacaagaggaatcgaaaactggggccccggactcctggtcaccgtctcctca

SEQ ID NO: 576 - DNA, 324 bp, synthetic construct
  misc_feature 1..324: note = CA4 VL Nucleotide Sequence
  source 1..324: mol_type = other DNA
gacatccaggtgacccagtctccgtcctccctgtctgcatctctaacagagagagtctccatcacttgccggaccagtcagagcgttaacaattacttaagctggtatcagcagaaaccagggcaagctcctaagctcctgatctattatgcaaccagattgtacaccgatgtcccatcccggttcagtggcagtggatctgggacagattacaccctcaccatcaccagcctggaggcggacgacactgcaacttattactgtctacaatatgatagtacacctcttgcattcggcggtgggaccaacgtggaaatcaaacgg

SEQ ID NO: 577 - DNA, 366 bp, synthetic construct
  misc_feature 1..366: note = 3bG4 VH Nucleotide Sequence
  source 1..366: mol_type = other DNA
caggtgcagcttcaggagtcgggacccagcctggtgaagccctcacagaccctctccctcacctgcacaatctctggattctcattaatcagcaatggtgtaggctgggtccgccaggctccaggaaaggtgccggagtgggttggtgatattgctagtagtggaaaggcatacagtaacccggccctgaaatcccggctcagcatcaccagggacacctccaagagccaagtctccctgtcactgaggagcgtgacaactgaggacacggccgtgtactactgtgtaagatgcagggatggtggtgtgacttatggttatgatatcgactactggggcccaggactcctggtcaccgtctcctca

SEQ ID NO: 578 - DNA, 333 bp, synthetic construct
  misc_feature 1..333: note = 3bG4 VL Nucleotide Sequence
  source 1..333: mol_type = other DNA
caggctgtgctgactcagccgtcatccgtgtccaagtccctgggccagagtgtctccatcacctgctccggaagcactagcaacgttggaagtggtaatgatgtgagctggttccaacaggtcccaggatcagcccccaaactcctcttctacggtgcaaccaaccgagcctcgggggtccccgaccgattctccggctccaggtctggcaacacagcgactctgaccatcacctcgcttcaggctgaggacgaggccgattattactgtggatcttatgacagcaatagcggtggtattttcggcagtgggaccaggctgaccgtcctgggt

SEQ ID NO: 579 - DNA, 375 bp, synthetic construct
  misc_feature 1..375: note = CA7 VH Nucleotide Sequence
  source 1..375: mol_type = other DNA
cgggtgcggctgcaggggtcgggacccagcctggtgaaaccttcacagaccctctccctcacctgcacggtctctggattctcttttgacagctattatgtaggctgggtccgccaggctccaggaaaggcactggagtggcttggtaatatatatagtactggaagggcattctataacccggccctgaaatcccggctcagcatcaccagggacacctccaagagccaagtctccctatcagtgagcagcgtgacaattgaggacacggccctgtactactgtgtcagaggctcgtattatcacggtggtggcaatgggatggtcgactttttcgactactggagcccaggactcctggtcaccgtctcctca

SEQ ID NO: 580 - DNA, 333 bp, synthetic construct
  misc_feature 1..333: note = CA7 VL Nucleotide Sequence
  source 1..333: mol_type = other DNA
caggtcgtgcggactcagccgtcctccgtgtctgggtccctgggccagagggtctccatcacctgctctggaagcagcagcaatgttggatatggtaattatgtgggctggttccaacaggtgccagggtcagcccccaaactcctcatctatgctgcaaccagtcgagcctcgggggtccccgaccgattctccggctccaggtctgggaatacagccaccctgaccatcgactcgctccaggctgaggacgaggccgattattactgttcatcttatcaacgcggtaacactggtgttttcggcagcgggaccaggctgaccgtcctgggt

SEQ ID NO: 581 - DNA, 351 bp, synthetic construct
  misc_feature 1..351: note = E2E8 VH Nucleotide Sequence
  source 1..351: mol_type = other DNA
caggtgcagctgcaggagtcgggacccagcctggtgaagccctcacagaccctctccctcacctgcacggtctctggattctcattaaccgaccgtggtgtagcctgggtccgccaggctccaggaaaggcactggagtgggttggtactatgcgtagtggtggaacgatagactataacccggccctgaaatcccggctcagcatcaccagggacacctccaagagccaagttttcctgtcactgagcagcgtcacaactgaggacatggccatgtactactgtgccagaggttatttgagcggtgatcgttatgcctggggccgaggactcctggtcaccgtctcctca

SEQ ID NO: 582 - DNA, 333 bp, synthetic construct
  misc_feature 1..333: note = E2E8 VL Nucleotide Sequence
  source 1..333: mol_type = other DNA
caggctgtgctgactcagccgtcctccgtgtccaagtccctgggccagagtgtctccatcgcctgctctggaagcaggagcgacattggatatggtaattatgtgagctggttccaacagatcccaggatcagcccccaaactccttatttatgatacaaacactcgggcctcgggggtccccgaccgattctccggcgccaggtctggcaacacagcaacactgaccatcaactcgctccaggctgaggacgaggccgattattactgtgcaaatattgacagtagtcgcagtcatcttttcggcagtggcaccagactgaccgtcctgggt

SEQ ID NO: 583 - DNA, 348 bp, synthetic construct
  misc_feature 1..348: note = 412E10 VH Nucleotide Sequence
  source 1..348: mol_type = other DNA
caggtgcagctgcaggagtcgggacccagcctggtgaagccctcacagaccctctccctcacctgcacggtctctggcttctctgtaataagcgattctgtagcctgggtccgccaggctccaggaaaagtgccggagtggcttggtgctagcggcagttctggaaacaaatactataacccggccctaaaatcccggctcagcatcaccagggacacctccaagagccaagtctccctgtcactgagcagcgtgacaactgaggatacggccgtgtactactgtgcgagaggtattatcgccggtgtagatgtctggggccgaggactcctggtcaccgtctcctca

SEQ ID NO: 584 - DNA, 330 bp, synthetic construct
  misc_feature 1..330: note = 412E10 VL Nucleotide Sequence
  source 1..330: mol_type = other DNA
caggctgtgctgactcagccgtcctccgtgtctgggtccctgggccagagggtctccatcacctgctctggaagcagcagcaacgttggatatggtaattatgtgggctggtaccaacaggtcccaggatcagcccccaaactcctcatctatggtacagccattcgagcctcgggggtccccgaccgattctccggctccaggtctggggacacagccacccttaccatcacctcgctccaggctgaggacgaggccgattactactgtgcatcttatcagagtaattacgcttttttcggcagcgggaccaggctgaccgtcctgggt

SEQ ID NO: 585 - DNA, 372 bp, synthetic construct
  misc_feature 1..372: note = CE2 VH Nucleotide Sequence
  source 1..372: mol_type = other DNA
caggtgcagctgcaggagtcgggacccagcctggtgaagccctcacagaccctctccctcacctgcacggtctctggattctcattaaccaactatcctgtaggctgggtccgccaggctccaggaaaggcactggagtggattggtaacatagaaaatgatggaagtgcgaactatgcctcggccctgaaatcccgactcagcatcaccagggacacctccaagaaccaagtctccctgtcactgagcagcgcgacaactgaggacacggccgtttactactgtggaagagaattcggtgggagtgatggttatacttatttcgttgatatcgactactggggcccaggactcctggtcaccgtctcctca

SEQ ID NO: 586 - DNA, 333 bp, synthetic construct
  misc_feature 1..333: note = CE2 VL Nucleotide Sequence
  source 1..333: mol_type = other DNA
caggctgtgctgactcagccgtcctccgtgtccaagtccctgggccagagtgtctccatcacctgctctggaagcagcagcaacgttggatatggtaattatgtgagctggttccaacaggtcccaggatcagcccccaaaatcctcatctatggtgcaaccagtcgagcctcgggggtccccgaccgattctccggctccaggtctggcaacacagcgactctgaccatcacctcgctccaggctgaggacgaggccgattattactgtgcatcttatgacggcagtagcagtggtgttttcggcagcgggaccaggctgaccgtcctgggt

SEQ ID NO: 587 - DNA, 372 bp, synthetic construct
  misc_feature 1..372: note = CE3 VH Nucleotide Sequence
  source 1..372: mol_type = other DNA
caggtgcgactgcaggagtcgggacccagcctggtgaagccctcacagaccctctccgtcacctgcacggtctctggattctcattgatcagcaatgctgtaggctgggtccgccaggctccaggaaaggtgccggagtcgcttgctggttgtagcagtgatggaaagtgttactataactcggccctgaaatcccggctcgacatcaccagggacacctcgaagaaccagatctccctgtcactgagcagcgtcacaactgacgacgcggccgtgtactactgtacaagaggctattatcctgtttatggttatgactatcttggcactatcgactactggggccccggactcctggtcaccgtctcctca

SEQ ID NO: 588 - DNA, 333 bp, synthetic construct
  misc_feature 1..333: note = CE3 VL Nucleotide Sequence
  source 1..333: mol_type = other DNA
caagctgtgctgactcaaccgtcctccgtgtctgggtccctgggccagagggtctccatcacctgctctggaagcagcagcaacgttggtagaaatgatgtagcctggttccaacaactcccaggatcaggcctcagaaccatcatctatggtactaccagtcgaccctcaggtatcccggaccgattctccggctccaagtctggcgttacggccaccctgaccatcgactcgctccaggctgaggacgaggccgattatttctgtgcctctggtgacagtagtgccattaatgatattttcggcagcgggaccaggctgaccgtcctgggt

SEQ ID NO: 589 - protein, 430 aa, Mus musculus
MADPRQEFDTMEDHAGDYTLLQDQEGDMDHGLKESPPQPPADDGAEEPGSETSDAKSTPTAEDVTAPLVDERAPDKQAAAQPHTEIPEGITAEEAGIGDTPNQEDQAAGHVTQARVASKDRTGNDEKKAKGADGKTGAKIATPRGAASPAQKGTSNATRIPAKTTPSPKTPPGSGEPPKSGERSGYSSPGSPGTPGSRSRTPSLPTPPTREPKKVAVVRTPPKSPSASKSRLQTAPVPMPDLKNVRSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 590 - protein, 13 aa, synthetic construct
  REGION 1..13: note = CE2 scAb binding region on hT40L 319-331: Parent
TSKCGSLGNIHHK

SEQ ID NO: 591 - protein, 13 aa, synthetic construct
  REGION 1..13: note = CE2 scAb binding region on hT40L 319-331: 322A
TSKAGSLGNIHHK

SEQ ID NO: 592 - protein, 13 aa, synthetic construct
  REGION 1..13: note = CE2 scAb binding region on hT40L 319-331: 323A
TSKCASLGNIHHK

SEQ ID NO: 593 - protein, 13 aa, synthetic construct
  REGION 1..13: note = CE2 scAb binding region on hT40L 319-331: 324A
TSKCGALGNIHHK

SEQ ID NO: 594 - protein, 13 aa, synthetic construct
  REGION 1..13: note = CE2 scAb binding region on hT40L 319-331: 325A
TSKCGSAGNIHHK

SEQ ID NO: 595 - protein, 13 aa, synthetic construct
  REGION 1..13: note = CE2 scAb binding region on hT40L 319-331: 326A
TSKCGSLANIHHK

SEQ ID NO: 596 - protein, 13 aa, synthetic construct
  REGION 1..13: note = CE2 scAb binding region on hT40L 319-331: 327A
TSKCGSLGAIHHK

SEQ ID NO: 597 - protein, 13 aa, synthetic construct
  REGION 1..13: note = CE2 scAb binding region on hT40L 319-331: 328A
TSKCGSLGNAHHK

SEQ ID NO: 598 - protein, 13 aa, synthetic construct
  REGION 1..13: note = S1D12 scAb and ME12 scAb binding region on hT40: 341-353: Parent
SEKLDFKDRVQSK

SEQ ID NO: 599 - protein, 13 aa, synthetic construct
  REGION 1..13: note = S1D12 scAb and ME12 scAb binding region on hT40: 341-353: 341A
AEKLDFKDRVQSK

SEQ ID NO: 600 - protein, 13 aa, synthetic construct
  REGION 1..13: note = S1D12 scAb and ME12 scAb binding region on hT40: 341-353: 342A
SAKLDFKDRVQSK

SEQ ID NO: 601 - protein, 13 aa, synthetic construct
  REGION 1..13: note = S1D12 scAb and ME12 scAb binding region on hT40: 341-353: 343A
SEALDFKDRVQSK

SEQ ID NO: 602 - protein, 13 aa, synthetic construct
  REGION 1..13: note = S1D12 scAb and ME12 scAb binding region on hT40: 341-353: 344A
SEKADFKDRVQSK

SEQ ID NO: 603 - protein, 13 aa, synthetic construct
  REGION 1..13: note = S1D12 scAb and ME12 scAb binding region on hT40: 341-353: 345A
SEKLAFKDRVQSK

SEQ ID NO: 604 - protein, 13 aa, synthetic construct
  REGION 1..13: note = S1D12 scAb and ME12 scAb binding region on hT40: 341-353: 346A
SEKLDAKDRVQSK

SEQ ID NO: 605 - protein, 13 aa, synthetic construct
  REGION 1..13: note = S1D12 scAb and ME12 scAb binding region on hT40: 341-353: 347A
SEKLDFADRVQSK

SEQ ID NO: 606 - protein, 13 aa, synthetic construct
  REGION 1..13: note = S1D12 scAb and ME12 scAb binding region on hT40: 341-353: 348A
SEKLDFKARVQSK

SEQ ID NO: 607 - protein, 13 aa, synthetic construct
  REGION 1..13: note = S1D12 scAb and ME12 scAb binding region on hT40: 341-353: 349A
SEKLDFKDAVQSK

SEQ ID NO: 608 - protein, 13 aa, synthetic construct
  REGION 1..13: note = S1D12 scAb and ME12 scAb binding region on hT40: 341-353: 350A
SEKLDFKDRAQSK

SEQ ID NO: 609 - protein, 13 aa, synthetic construct
  REGION 1..13: note = S1D12 scAb and ME12 scAb binding region on hT40: 341-353: 351A
SEKLDFKDRVASK

SEQ ID NO: 610 - protein, 13 aa, synthetic construct
  REGION 1..13: note = S1D12 scAb and ME12 scAb binding region on hT40: 341-353: 352A
SEKLDFKDRVQAK

SEQ ID NO: 611 - protein, 13 aa, synthetic construct
  REGION 1..13: note = S1D12 scAb and ME12 scAb binding region on hT40: 341-353: 353A
SEKLDFKDRVQSA

SEQ ID NO: 612 - protein, 13 aa, synthetic construct
  REGION 1..13: note = CA4 scAb binding region on hT40: 355-367: Parent
GSLDNITHVPGGG

SEQ ID NO: 613 - protein, 13 aa, synthetic construct
  REGION 1..13: note = CA4 scAb binding region on hT40: 355-367: 358A
GSLANITHVPGGG

SEQ ID NO: 614 - protein, 13 aa, synthetic construct
  REGION 1..13: note = CA4 scAb binding region on hT40: 355-367: 359A
GSLDAITHVPGGG

SEQ ID NO: 615 - protein, 13 aa, synthetic construct
  REGION 1..13: note = CA4 scAb binding region on hT40: 355-367: 360A
GSLDNATHVPGGG

SEQ ID NO: 616 - protein, 13 aa, synthetic construct
  REGION 1..13: note = CA4 scAb binding region on hT40: 355-367: 361A
GSLDNIAHVPGGG

SEQ ID NO: 617 - protein, 13 aa, synthetic construct
  REGION 1..13: note = CA4 scAb binding region on hT40: 355-367: 362A
GSLDNITAVPGGG

SEQ ID NO: 618 - protein, 13 aa, synthetic construct
  REGION 1..13: note = CA4 scAb binding region on hT40: 355-367: 363A
GSLDNITHAPGGG

SEQ ID NO: 619 - protein, 13 aa, synthetic construct
  REGION 1..13: note = CA4 scAb binding region on hT40: 355-367: 364A
GSLDNITHVAGGG

SEQ ID NO: 620 - protein, 13 aa, synthetic construct
  REGION 1..13: note = S1G2 and related scAbs binding region on hT40: 367-379: Parent
GNKKIETHKLTFR

SEQ ID NO: 621 - protein, 13 aa, synthetic construct
  REGION 1..13: note = S1G2 and related scAbs binding region on hT40: 367-379: 367A
ANKKIETHKLTFR

SEQ ID NO: 622 - protein, 13 aa, synthetic construct
  REGION 1..13: note = S1G2 and related scAbs binding region on hT40: 367-379: 368A
GAKKIETHKLTFR

SEQ ID NO: 623 - protein, 13 aa, synthetic construct
  REGION 1..13: note = S1G2 and related scAbs binding region on hT40: 367-379: 369A
GNAKIETHKLTFR

SEQ ID NO: 624 - protein, 13 aa, synthetic construct
  REGION 1..13: note = S1G2 and related scAbs binding region on hT40: 367-379: 370A
GNKAIETHKLTFR

SEQ ID NO: 625 - protein, 13 aa, synthetic construct
  REGION 1..13: note = S1G2 and related scAbs binding region on hT40: 367-379: 371A
GNKKAETHKLTFR

SEQ ID NO: 626 - protein, 13 aa, synthetic construct
  REGION 1..13: note = S1G2 and related scAbs binding region on hT40: 367-379: 372A
GNKKIATHKLTFR

SEQ ID NO: 627 - protein, 13 aa, synthetic construct
  REGION 1..13: note = S1G2 and related scAbs binding region on hT40: 367-379: 373A
GNKKIEAHKLTFR

SEQ ID NO: 628 - protein, 13 aa, synthetic construct
  REGION 1..13: note = S1G2 and related scAbs binding region on hT40: 367-379: 374A
GNKKIETAKLTFR

SEQ ID NO: 629 - protein, 13 aa, synthetic construct
  REGION 1..13: note = S1G2 and related scAbs binding region on hT40: 367-379: 375A
GNKKIETHALTFR

SEQ ID NO: 630 - protein, 13 aa, synthetic construct
  REGION 1..13: note = S1G2 and related scAbs binding region on hT40: 367-379: 376A
GNKKIETHKATFR

SEQ ID NO: 631 - protein, 13 aa, synthetic construct
  REGION 1..13: note = S1G2 and related scAbs binding region on hT40: 367-379: 377A
GNKKIETHKLAFR

SEQ ID NO: 632 - protein, 13 aa, synthetic construct
  REGION 1..13: note = S1G2 and related scAbs binding region on hT40: 367-379: 378A
GNKKIETHKLTAR

SEQ ID NO: 633 - protein, 13 aa, synthetic construct
  REGION 1..13: note = S1G2 and related scAbs binding region on hT40: 367-379: 379A
GNKKIETHKLTFA

SEQ ID NO: 634 - DNA, 24 bp, synthetic construct
  misc_feature 1..24: note = OvCHFOR
  source 1..24: mol_type = other DNA
gactttcggggctgtggtggaggc

SEQ ID NO: 635 - DNA, 32 bp, synthetic construct
  misc_feature 1..32: note = OvCKFOR
  source 1..32: mol_type = other DNA
gatggtttgaagagggagacggatggctgagc

SEQ ID NO: 636 - DNA, 27 bp, synthetic construct
  misc_feature 1..27: note = OvCLFOR
  source 1..27: mol_type = other DNA
acagggtgaccgagggtgcggacttgg